(19) United States
(12) Patent Application Publication
(10) Pub. No.: US 2026/0137706 A1
(43) Pub. Date: May 21, 2026

(54) METHODS OF TREATING INFLAMMATORY DISEASES

(71) Applicant: INTERLINE THERAPEUTICS, INC., Brisbane, CA (US)

(72) Inventors: Mario G. CARDOZO, San Francisco, CA (US); Zuojun GUO, Pleasanton, CA (US); Robert Joseph MOREAU, Walnut Creek, CA (US); Naomi S. RAJAPAKSA, Belmont, CA (US); Meera RAO, San Francisco, CA (US); David C. TULLY, Danville, CA (US); Steffen GREßIES, Berlin (DE); Kai THEDE, Berlin (DE); Zachary K. SWEENEY, Redwood City, CA (US)

(21) Appl. No.: 19/411,060

(22) Filed: Dec. 5, 2025

Related U.S. Application Data
(63) Continuation of application No. PCT/US24/33121, filed on Jun. 7, 2024.
(60) Provisional application No. 63/472,252, filed on Jun. 9, 2023.
(60) Provisional application No. 63/609,168, filed on Dec. 12, 2023.

(51) Int. Cl. A61K 31/675; A61K 31/437; A61K 31/4375; A61K 31/444; A61K 31/4545; A61K 31/4709; A61K 31/4725; A61K 31/496; A61K 31/4985; A61K 31/5025; A61K 31/519; A61K 31/5377; A61K 31/5383; C07B 59/00; C07D 471/04; C07D 487/04; C07D 519/00; C07F 9/6561; C07F 9/6584

(52) U.S. Cl. CPC A61K 31/675; A61K 31/437; A61K 31/4375; A61K 31/444; A61K 31/4545; A61K 31/4709; A61K 31/4725; A61K 31/496; A61K 31/4985; A61K 31/5025; A61K 31/519; A61K 31/5377; A61K 31/5383; C07B 59/002; C07D 471/04; C07D 487/04; C07D 519/00; C07F 9/6561; C07F 9/6584; C07B 2200/05

(57) ABSTRACT

The present disclosure relates to compounds of Formula (I), as defined herein, and pharmaceutically acceptable salts thereof, and compositions comprising same. Also described are methods of treating the diseases and disorders disclosed herein, with the compounds of Formula (I), and pharmaceutically acceptable salts thereof, and the compositions comprising same.

This application is a continuation application of International Patent Application No. PCT/US2024/033121 filed on Jun. 7, 2024, which claims priority to U.S. Provisional Application No. 63/472,252 filed on Jun. 9, 2023, and U.S. Provisional Application No. 63/609,168 filed on Dec. 12, 2023; the entire contents of which are hereby incorporated by reference.

### FIELD

The present application relates to the fields of chemistry and biology, in particular to compounds of Formula (I), as defined herein, and pharmaceutically acceptable salts thereof, and compositions comprising same. Also described are methods of treating the diseases and disorders disclosed herein, with the compounds of Formula (I), and pharmaceutically acceptable salts thereof, and the compositions comprising same.

### BACKGROUND

Receptor interacting protein kinase 2 (RIPK2) is a serine-threonine protein kinase, and is a signaling molecule downstream of nucleotide-binding oligomerization domain 1 (NOD1), NOD2, and Toll-like receptors (TLRs). The RIPK2 protein includes a kinase domain (KD), an intermediate domain (INTD), and a caspase activation and recruitment domain (CARD). The CARD domain of RIPK2 mediates interaction with NOD1 and NOD2. RIPK2 is expressed in the cytoplasm of antigen-presenting cells including dendritic cells and macrophages, and is also expressed in T cells and epithelial cells.

NOD receptors function in the innate immune system, detecting bacterial pathogens by binding to diaminopimelic acid or muramyl dipeptide residues present in bacterial peptidoglycans. Interactions between RIPK2 and NOD1, NOD2 and TLRs trigger the release of pro-inflammatory cytokines including TNF-α, IL-6, and IL-12/23p40, and RIPK2-mediated induction of NF-kappa-B-dependent inflammatory responses. Activation of RIPK2 and dysregulation of the RIPK2-NOD signaling pathways may also have a role in the pathogenesis of various inflammatory diseases. RIPK2 has been reported to be a prognostic indicator and candidate therapeutic target for various cancers.

### SUMMARY

Some embodiments provide a compound of Formula (I):

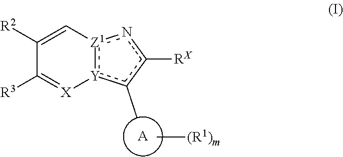

or a pharmaceutically acceptable salt thereof, wherein:
one or two of X, Y, and Z¹ is independently N and the other of X, Y, and Z¹ is C;
each [symbol] is a single bond or a double bond;
R^{X} is hydrogen, —NH₂, or halogen; Ring A is phenyl or 5-10 membered heteroaryl;
m is 0, 1, 2, 3, or 4;
each R¹ is independently:
(i) C1-C6 alkoxy optionally substituted with hydroxyl or phenyl,
(ii) C1-C6 deuteroalkoxy,
(iii) C1-C6 alkoxyalkyl,
(iv) 5-6 membered heteroaryl,
(v) —N(R^{1A})—S(O₂)R^{1B},
(vi) —(C═O)NR^{1A}R^{1B},
(vii) halogen,
(viii) cyano,
(ix) hydroxyl,
(x) —NR^{A}R^{B},
(xi) C1-C6 alkyl optionally substituted with 1-2 substituents independently selected from hydroxyl and 4-8 membered heterocyclyl optionally substituted with hydroxyl or C1-C6 alkyl,
(xii) C1-C6 haloalkyl,
(xiii) C1-C6 haloalkoxy,
(xiv) C3-C6 cycloalkyl,
(xv) 4-8 membered heterocyclyl optionally substituted with 1-2 substituents independently selected from hydroxyl, C1-C6 alkyl, C1-C6 haloalkyl, and —(C═O)OC1-C6 alkyl,
(xvi) —S(O₂)C1-C6 alkyl, or
(xvii) 4-10 membered heterocyclyloxy optionally substituted with acyl,
(xviii) phenyl;
each R^{1A} is independently hydrogen or C1-C6 alkyl;
each R^{1B} is independently
(i) C1-C6 alkyl optionally substituted with C3-C6 cycloalkyl, 5-6 membered heteroaryl optionally substituted with C1-C6 alkyl, or 4-10 membered heterocyclyl optionally substituted with —(C═O)OC1-C6 alkyl,
(ii) 5-10 membered heteroaryl optionally substituted with 1-3 substituents independently selected from C1-C6 alkyl, hydroxyl, and C1-C6 hydroxyalkyl,
(iii) C3-C6 cycloalkyl optionally substituted with 1-2 substituents independently selected from C1-C6 alkyl and C1-C6 hydroxyalkyl,
(iv) ethylenyl,
(v) C1-C6 haloalkyl,
(vi) 4-10 membered heterocyclyl optionally substituted with —(C═O)OC1-C6 alkyl,
(vii) —NR^{1A}R^{1A}, or
(viii) phenyl;
R^{A} is hydrogen or C1-C6 alkyl;
R^{B} is
   (i) hydrogen,
   (ii) —S(O₂)C1-C6 alkyl,
   (iii) C3-C6 cycloalkyl optionally substituted with hydroxyl or C1-C6 alkoxy,
   (iv) —(C═O)C1-C6 alkyl,
   (v) —(C═O)OC1-C6 alkyl,
   (vi) 4-8 membered heterocyclyl optionally substituted with hydroxyl, or
   (vii) C1-C6 alkyl optionally substituted with 1-4 substituents independently selected from: halogen, hydroxyl, —NR^{C}R^{D}, C1-C6 alkoxy, C1—C6 haloalkoxy, C3-C6 cycloalkyl, phenyl optionally substituted with C1-C6 alkoxy, 5-6 membered heteroaryl optionally substituted with C1-C6 alkyl, and 4-8 membered heterocyclyl optionally substituted with —C(═O)C1-C6 alkyl or C1-C6 alkyl,
   (viii) —(C═O)5- or 6-membered heteroaryl optionally substituted with C1-C6 alkyl;
R² is
(i) hydrogen,
(ii) halogen,
(iii) C1-C6 alkoxy optionally substituted with 1-3 substituents independently selected from
   (a) hydroxyl,
   (b) halogen,
   (c) phosphate,
   (d) —NR^{2A}R^{2B},
   (e) 4-10 membered heterocyclyl optionally substituted with 1-3 substituents independently selected from hydroxyl, C1-C6 alkyl, C1-C6 haloalkyl, C1-C6 hydroxyalkyl, C1-C6 alkoxy, and C1-C6 alkoxyalkyl,
   (f) 5-6 membered heteroaryl optionally substituted with C1-C6 alkyl,
   (g) C3-C6 cycloalkyl optionally substituted with hydroxyl or hydroxyalkyl,
   (h) CO₂H,
   (i) C(O)R^{2A}
   (j) C1-C6 alkoxy; or
   (k) oxo;
(iv) C1-C6 haloalkoxy;
(v) 4-10 membered heterocyclyloxy,
(vi) —(C═O)NR^{2A}R^{2B},
(vii) C1-C6 alkyl optionally substituted with 1-3 substituents independently selected from hydroxyl, halogen, and —NR^{2A}R^{2B},
(viii) 5-6 membered heteroaryl optionally substituted with C1-C6 alkyl or C1-C6 alkoxy, or
(ix) —NR^{2A}R^{2B};
each R^{2A} and R^{2B} are independently hydrogen, C1-C6 alkyl optionally substituted with hydroxyl, or C1-C6 hydroxyalkyl;
R³ is
   (i) C1-C6 thioalkyl,

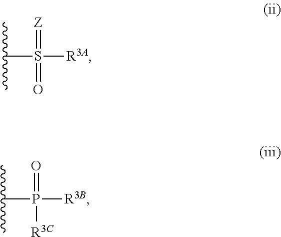

(iv) 4-8 membered heterocyclyl optionally substituted with 1-3 substituents independently selected from halogen, hydroxyl, C1-C6 alkyl, and C1-C6 alkoxy,
(v) C1-C6 alkyl optionally substituted with NR^{E}R^{F} or hydroxyl,
(vi) —CO₂H,
(vii) —C(═O)NR^{E}R^{F},
(viii) C1-C6 alkoxy optionally substituted with 4-10 membered heterocyclyl optionally substituted with C1-C6 alkoxy,
(ix) hydrogen,
(x) C1-C6 haloalkyl,
(xi) 5-6 membered heteroaryl optionally substituted with C1-C6 alkyl,
(xii) C1-C6 alkoxyalkyl, or
(xiii) C1-C6 hydroxyalkyl;
Z is O or NR⁴;
R^{3A} is
(i) C1-C6 haloalkyl,
(ii) C3-C6 cycloalkyl optionally substituted with C1-C6 alkyl,
(iii) C1-C6 alkyl optionally substituted with
   (a) C3-C6 cycloalkyl,
   (b) 5-6 membered heteroaryl optionally substituted with C1-C6 alkyl,
   (c) hydroxyl,
   (d) C1-C6 alkoxy, or
   (e) 4-6 membered heterocyclyl optionally substituted with 4-6 membered heterocyclyl or C1-C6 alkyl optionally substituted with C1-C6 alkoxy,
(iv) C1-C6 alkoxyalkyl,
(v) C1-C6 hydroxyalkyl,
(vi) 5-6 membered heteroaryl optionally substituted with C1-C6 alkyl, or
(vii) 4-10 membered heterocyclyl optionally substituted with 1-3 substituents independently selected from hydroxyl, C1-C6 alkyl, —C(O)OC1-C6 alkyl, and C1-C6 alkoxy,
(viii) —N(C1-C6 alkyl)₂;
R^{3B} and R^{3C} are each independently C3-C6 cycloalkyl or C1-C6 alkyl optionally substituted with C3-C6 cycloalkyl, or
R^{3B} and R^{3C} with the atom to which they are attached together form a 4-8 membered heterocyclyl optionally substituted with C1-C6 alkyl;
R⁴ is hydrogen or C1-C6 alkyl; and
each R^{C} and R^{D} are each independently hydrogen, —(C═O)C1-C6 alkyl, or C1-C6 alkyl optionally substituted with oxo;
each R^{E} and R^{F} are each independently hydrogen or C1-C6 alkyl, or R^{E} and R^{F} with the atom to which they are attached together form a 4-8 membered heterocyclyl optionally substituted with hydroxyl.

Also provided herein is a method of treating a RIPK2-associated disease or disorder in a subject in need thereof, comprising administering to the subject an effective amount of a compound of Formula (I), or a pharmaceutically acceptable salt thereof, (e.g., a compound of Formula (I), Formula (I-a), Formula (I-b), Formula (I-c), Formula (I-d), Formula (I-e), Formula (I-f), Formula (I-g), Formula (I-h), or Formula (I-i), Formula (I-j), Formula (I-k), Formula (I-l), or Formula (I-m), or a pharmaceutically acceptable salt of any of the foregoing), or a pharmaceutical composition comprising subject an effective amount of a compound of Formula (I), or a pharmaceutically acceptable salt thereof.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety, unless expressly indicated otherwise. In case of conflict, the present specification, including definitions, will control.

Other features and advantages of the disclosure will be apparent from the following detailed description and figures, and from the claims.

### DETAILED DESCRIPTION

Biologics and small molecules targeting pro-inflammatory signaling pathways have been used to successfully treat inflammatory and other diseases in patients, however, a significant fraction of patients are refractory to existing therapies. Therefore, there exists a need for identification of novel therapeutic molecules that modulate or inhibit these pathways, such as the compounds of Formula (I), and pharmaceutically acceptable salts thereof described herein.

### Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties. In case of conflict, the present specification, including definitions, will control.

The term “about” when referring to a number or a numerical range means that the number or numerical range referred to is an approximation, for example, within experimental variability and/or statistical experimental error, and thus the number or numerical range may vary up to ±10% of the stated number or numerical range.

The phrase “therapeutically effective amount” or “effective amount” means an amount of compound that, when administered to a subject in need of such treatment, is sufficient to (i) treat a disease or disorder as described herein (e.g., a RIPK2-associated disease or disorder), (ii) attenuate, ameliorate, or eliminate one or more symptoms of the particular disease or disorder, or (iii) delay the onset of one or more symptoms of the particular disease or disorder described herein.

As used herein, terms “treat” or “treatment” refer to therapeutic or palliative measures. Beneficial or desired clinical results include, but are not limited to, alleviation, in whole or in part, of symptoms associated with a disease or disorder, diminishment of the extent of a disorder, stabilized (i.e., not worsening) state of a disease or disorder, delay or slowing of disease progression, amelioration or palliation of the disease state (e.g., one or more symptoms of the disease or disorder), and remission (whether partial or total), whether detectable or undetectable and can be determined by various clinical assessments including clinical evaluation and self-reporting. “Treatment” can also mean prolonging survival as compared to expected survival if not receiving treatment.

The term “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21^{st} ed.; Lippincott Williams & Wilkins: Philadelphia, PA, 2005; Handbook of Pharmaceutical Excipients, 6^{th} ed.; Rowe et al., Eds.; The Pharmaceutical Press and the American Pharmaceutical Association: 2009; Handbook of Pharmaceutical Additives, 3^{rd} ed.; Ash and Ash Eds.; Gower Publishing Company: 2007; Pharmaceutical Preformulation and Formulation, 2^{nd} ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid, and organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “pharmaceutically acceptable excipients”), such as stabilizers, diluents, dispersing agents, suspending agents, thickening agents, and/or other excipients. The pharmaceutical composition facilitates administration of the compound to an organism.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human. In some embodiments, the subject is a human.

The term “halo” or “halogen” refers to one of the halogens, group 17 of the periodic table. In particular the term refers to fluorine, chlorine, bromine and iodine. Preferably, the term refers to fluorine or chlorine.

The term “oxo” refers to a divalent doubly bonded oxygen atom (i.e., “=0”). As used herein, oxo groups are attached to carbon atoms to form carbonyls.

The term “alkyl” refers to a saturated acyclic hydrocarbon radical that may be a straight chain or branched, containing the indicated number of carbon atoms. For example, C₁₋₁₀ indicates that the group may have from 1 to 10 (inclusive) carbon atoms in it. Non-limiting examples include methyl, ethyl, iso-propyl, tert-butyl, n-hexyl.

The term “alkenyl” refers to an acyclic hydrocarbon radical that may be a straight chain or branched, containing the indicated number of carbon atoms and one or more carbon-carbon double bonds. Non-limiting examples include ethylenyl and allyl.

The term “haloalkyl” refers to an alkyl, in which one or more hydrogen atoms is/are replaced with an independently selected halogen.

The term “hydroxyalkyl” refers to an alkyl group as described herein, in which one or more hydrogen atoms is/are replaced with one or more hydroxyl groups, as described herein.

The term “alkoxy” refers to an —O-alkyl radical (e.g., —OCH₃).

The term “thioalkyl” refers to an alkyl group as described herein, which is attached to a molecule via a sulfur atom (e.g., —SCH₃).

The term “haloalkoxy” refers to a haloalkyl group which is attached to a molecule via an oxygen atom (e.g., —OCF₃).

The term “alkoxyalkyl” refers to an alkyl group as described herein, in which one or more hydrogen atoms is/are replaced with one or more alkoxy groups as described herein.

As used herein, the term “cyano” refers to a —CN radical.

As used herein, the term “hydroxyl” refers to an —OH radical.

As used herein, the term “amino” refers to a —NH₂ radical.

As used herein, the term “phosphate” refers to a —P(═O)₂(OH)₂ radical.

As used herein, the term “heteroaryl” refers to a 5-14 membered mono-, bi-, or tricyclic group wherein at least one ring in the system is aromatic; and wherein one or more carbon atoms in at least one ring in the system is/are replaced with an heteroatom independently selected from the group consisting of N, O, S, B, Si, and P. For example, there may be 1, 2 or 3 heteroatoms, optionally 1 or 2. A heteroaryl may further contain one or more oxo, N-oxide, S-oxide, and/or S,S-dioxide groups, valence permitting. In heteroaryl groups with one aromatic ring, the aromatic ring does not have to contain the heteroatom(s). Non-limiting examples of heteroaryl groups include furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, 2-pyridone, pyridazine, pyrimidine, pyrazine, purine, pteridine, quinoline, isoquinoline, quinazoline, quinoxaline, cinnoline, triazine, 2,3-dihydro-1H-pyrrolo[2,3-b]pyridine, 3,4-dihydro-2H-pyrido[3,2-b][1,4]oxazine, 1′,2′-dihydrospiro[cyclopropane-1,3′-pyrrolo[2,3-b]pyridine], and 3′,4′-dihydrospiro [cyclopropane-1,2′-pyrido[3,2-b][1,4]oxazine].

For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridone

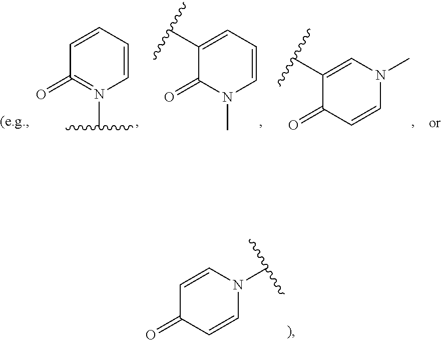

pyrimidone

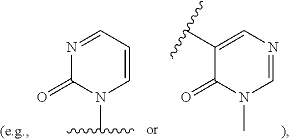

pyridazinone

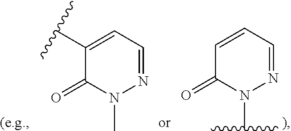

pyrazinone

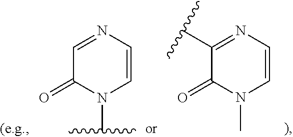

and imidazolone

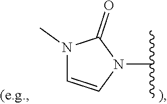 [SMILES: *n1ccn(C)c1=O]

wherein each ring nitrogen adjacent to a carbonyl is tertiary (i.e., the oxo group (i.e., “═O”) herein is a constituent part of the heteroaryl ring).

As used herein, the term “cycloalkyl” refers to a saturated or partially unsaturated mono-, bi-, or tricyclic carbon group having 3 to 20 carbon atoms. Bicyclic and tricyclic cycloalkyl groups include fused, spiro, and bridged ring systems. Non-limiting examples of cycloalkyl groups include cyclopropyl, cyclohexyl, spiro[2.3]hexyl, and bicyclo[1.1.1]pentyl.

The term “cycloalkoxy” refers to an —O-cycloalkyl radical (e.g., —O-cyclopropyl).

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system. Examples of aryl groups include phenyl, naphthyl, tetrahydronaphthyl, and the like.

The term “heterocyclyl” refers to a saturated or partially unsaturated hydrocarbon monocyclic, bicyclic, or tricyclic ring system having from 3 to 20 ring atoms, that is not aromatic, and having at least one heteroatom within the ring system selected from the group consisting of N, O, S, B, Si, and P. Bicyclic and tricyclic heterocyclyl groups include fused, spiro, and bridged ring systems. A heterocyclyl group may be denoted as a “5 to 10 membered heterocyclyl group,” which is a ring system containing 5, 6, 7, 8, 9 or 10 atoms at least one being a heteroatom. A heterocycle may further contain one or more oxo, thiocarbonyl, N-oxide, S-oxide, and/or S,S-dioxide groups, valence permitting, so as to make the definition include oxo-systems and thio-systems such as lactams, lactones, cyclic imides, cyclic thioimides and cyclic carbamates. A heterocyclyl group may be bonded to the rest of the molecule through any carbon atom or through a heteroatom such as nitrogen. Exemplary heterocyclyl groups include, but are not limited to 1,3-dioxolane, 1,4-dioxolane, maleimide, succinimide, dioxopiperazine, hydantoin, imidazoline, imidazolidine, isoxazolidine, oxazoline, oxazolidine, oxazolidinone, thiazoline, thiazolidine, morpholine, oxirane, piperidine N-oxide, piperidine, piperazine, pyrrolidine, pyrrolidone, pyrrolidione, 4-piperidone, pyrazoline, pyrazolidine, 2-oxopyrrolidine, pyrrolidinyl, tetrahydrofuryl, thiolanyl, pyrazolinyl, oxathiolanyl, isoxazolidinyl, isothiazolidinyl, pyrrolinyl, pyrrolidinonyl, pyrazolidinyl, imidazolinyl, dioxolanyl, sulfolanyl, thiazolidedionyl, succinimidyl, dihydrofuranonyl, pyrazolidinonyl, oxazolidinyl, isoxazolidinonyl, hydantionyl, thiohydantionyl, imidazolidinonyl, oxazolidinonyl, thiazolidinonyl, oxathiolanonyl, dioxolanonyl, dioxazolidinonyl, oxadiazolidinonyl, triazolidinonyl, triazolidinethionyl, oxadiazolidinethionyl, dioxazolidinethionyl, dioxolanethionyl, oxazolidinethionyl, imidazolidinethionyl, isothiazolidinonyl, piperidinyl, tetrahydropyranyl, thianyl, morpholinyl, thiomorpholinyl, dioxanyl, piperazinyl, dithianyl, oxazinyl, tetrahydropyranonyl, piperidinonyl, dioxanonyl, oxazinanonyl, morpholinonyl, thiomorpholinonyl, piperazinonyl, tetrahydropyrimidinonyl, piperidinedionyl, oxazinanedionyl, dihydropyrimidindione, tetrahydropyridazinonyl, triazinanonyl, oxadiazinanonyl, dioxazinanonyl, morpholinedionyl, piperazinedionyl, piperazinetrionyl, triazinanedionyl and 2-azaspiro[3.3]heptanyl.

The term “saturated” as used in this context means only single bonds present between constituent atoms.

As used herein, when a ring is described as being “partially unsaturated”, it means said ring has one or more additional degrees of unsaturation (in addition to the degree of unsaturation attributed to the ring itself, e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

As used herein, the symbol [symbol] depicts the point of attachment of an atom or moiety to the indicated atom or group in the remainder of the molecule.

Whenever a group is described as being “optionally substituted” that group may be unsubstituted or substituted with one or more of the indicated substituents. When a group is substituted, that substitution can include the sharing of a carbon atom between the parent group and the substitution to form a spiro ring. For example, an n-butyl group substituted with cyclopropyl includes both

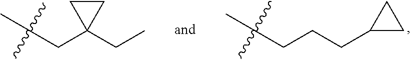

amongst others.

For the avoidance of doubt, and unless otherwise specified, for rings and cyclic groups (e.g., carbocycle, aryl, cycloalkyl, heterocyclyl, heteroaryl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

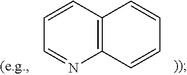

(ii) a single ring atom (spiro-fused ring systems)

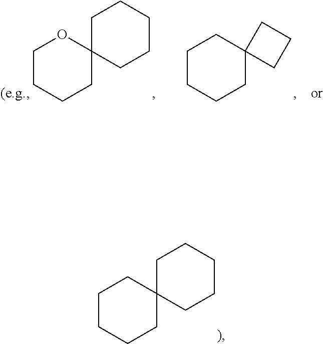

or (iii) a contiguous array of ring atoms (bridged ring systems having all bridge lengths >0)

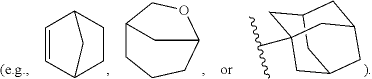

In addition, any compound or structure given herein, is also intended to represent unlabeled forms as well as isotopically labeled forms of the compounds. These forms of compounds are referred to as “isotopically enriched.” Isotopically enriched compounds have structures depicted herein, except that one or more atoms are replaced by an atom having a selected atomic mass or mass number.

Examples of isotopes that can be incorporated into the disclosed compounds include isotopes of hydrogen, carbon, nitrogen, oxygen, phosphorous, fluorine, chlorine and iodine, such as ²H, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically enriched compounds of the present disclosure, for example those into which radioactive isotopes such as ¹³C and ¹⁴C are incorporated. Such isotopically enriched compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term“isotopically enriched” compounds includes“deuterated” compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, particularly a human. Such compounds are synthesized by means known in the art, for example by employing starting materials in which one or more hydrogens have been replaced by deuterium. Indeed, isotopically enriched compounds of this disclosure can generally be prepared by carrying out the procedures disclosed in the schemes or in the examples and preparations described below by substituting a readily available isotopically enriched reagent for a non-isotopically enriched reagent.

Deuterium enriched compounds of the present disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index relative to the corresponding non-enriched compound.

The concentration of a heavier isotope, such as deuterium, may be defined by an isotopic enrichment factor. In some embodiments, the positions noted as “H” or “hydrogen” in the compounds described herein have hydrogen at its natural abundance isotopic composition. In some embodiments, the positions noted as “H” or “hydrogen” in the compounds described herein have hydrogen enriched in deuterium above its natural abundance isotopic composition, i.e., the compound is a deuterium enriched compound. Examples of deurated groups in the compounds described herein include, but are not limited to deuteromethine

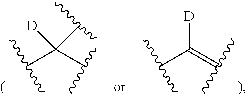

monodeuteromethylene

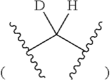 [SMILES: *C(*)[2H]]

and dideuteromethylene

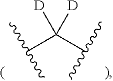 [SMILES: *C(*)([2H])[2H]]

trideuteromethyl

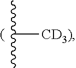

trideuteromethoxy

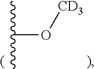

and the like. Compounds of the present disclosure also include deuterium enriched compounds at the alpha position of an oxo group, such as

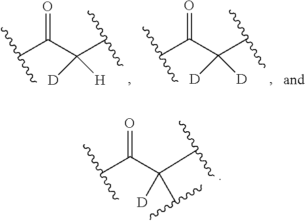 [SMILES: *C(=O)C(*)[2H] ; *C(=O)C(*)([2H])[2H] ; *C(=O)C(*)(*)[2H]]

In addition, the compounds generically or specifically disclosed herein are intended to include all tautomeric forms. Thus, by way of example, a compound containing the moiety:

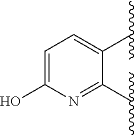 [SMILES: *c1ccc(O)nc1*]

encompasses the tautomeric form containing the moiety:

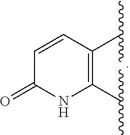 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The compounds provided herein may encompass various stereochemical forms. The compounds also encompass enantiomers (e.g., R and S isomers), diastereomers, as well as mixtures of enantiomers (e.g., R and S isomers) including racemic mixtures and mixtures of diastereomers, as well as individual enantiomers and diastereomers, which arise as a consequence of structural asymmetry in certain compounds. Unless otherwise indicated, when a disclosed compound is named or depicted by a structure without specifying the stereochemistry (e.g., a “flat” structure) and has one or more chiral centers, it is understood to represent all possible stereoisomers of the compound. Likewise, unless otherwise indicated, when a disclosed compound is named or depicted by a structure that specifies the stereochemistry (e.g., a structure with “wedge” and/or “dashed” bonds) and has one or more chiral centers, it is understood to represent the indicated stereoisomer of the compound.

The details of one or more embodiments of this disclosure are set forth in the accompanying drawings and the description below. Other features and advantages of the present disclosure will be apparent from the description and from the claims.

A “RIPK2 inhibitor” as defined herein includes any compound exhibiting RIPK2 inhibition activity. In some embodiments, a RIPK2 inhibitor is selective for RIPK2. Exemplary RIPK2 inhibitors can exhibit inhibition activity (IC₅₀) against a RIPK2 of less than about 1000 nM, less than about 500 nM, less than about 200 nM, less than about 100 nM, less than about 50 nM, less than about 25 nM, less than about 10 nM, less than about 5 nM, or less than about 1 nM as measured in an assay as described herein. In some embodiments, a RIPK2 inhibitor can exhibit inhibition activity (IC₅₀) against RIPK2 of less than about 25 nM, less than about 10 nM, less than about 5 nM, or less than about 1 nM as measured in an assay as provided herein.

### Compounds of Formula (I)

RIPK2 mediates inflammatory signaling via NOD1, NOD2, and TLRs. However, in vitro inhibition of RIPK2 kinase activity alone is not fully predictive of suppression of downstream cellular RIPK2 signaling. Rather, interfering with the interaction of RIPK2 and XIAP is necessary to robustly reduce inflammatory signaling. In some cases, potent inhibitors of RIPK2 kinase activity lack the ability to disrupt RIPK2/XIAP binding and thus lack the ability to completely block inflammatory signaling in cells or in human subjects. The present disclosure is based, in part, on the discovery that selected compounds described herein exert RIPK2 inhibition via simultaneous inhibition of RIPK2 kinase activity and disruption of the RIPK2/XIAP interaction and thus reduce cellular inflammatory signaling.

Some embodiments provide a compound of Formula (I):

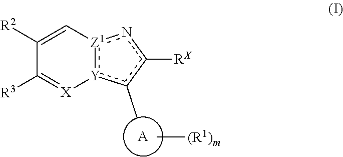

or a pharmaceutically acceptable salt thereof, wherein:
one or two of X, Y, and Z¹ is independently N and the other of X, Y, Z¹, and is C;
each [symbol] is a single bond or a double bond;
R^{X} is hydrogen, —NH₂, or halogen;
Ring A is phenyl or 5-10 membered heteroaryl;
m is 0, 1, 2, 3, or 4;
each R¹ is independently:
(i) C1-C6 alkoxy optionally substituted with hydroxyl or phenyl,
(ii) C1-C6 deuteroalkoxy,
(iii) C1-C6 alkoxyalkyl,
(iv) 5-6 membered heteroaryl,
(v) —N(R^{1A})—S(O₂)R^{1B},
(vi) —(C═O)NR^{1A}R^{1B},
(vii) halogen,
(viii) cyano,
(ix) hydroxyl,
(x) —NR^{A}R^{B},
(xi) C1-C6 alkyl optionally substituted with 1-2 substituents independently selected from hydroxyl and 4-8 membered heterocyclyl optionally substituted with hydroxyl or C1-C6 alkyl,
(xii) C1-C6 haloalkyl,
(xiii) C1-C6 haloalkoxy,
(xiv) C3-C6 cycloalkyl,
(xv) 4-8 membered heterocyclyl optionally substituted with 1-2 substituents independently selected from hydroxyl, C1-C6 alkyl, C1-C6 haloalkyl, and —(C═O)OC1-C6 alkyl,
(xvi) —S(O₂)C1-C6 alkyl, or
(xvii) 4-10 membered heterocyclyloxy optionally substituted with acyl,
(xviii) phenyl;
each R^{1A} is independently hydrogen or C1-C6 alkyl;
each R^{1B} is independently
(i) C1-C6 alkyl optionally substituted with C3-C6 cycloalkyl, 5-6 membered heteroaryl optionally substituted with C1-C6 alkyl, or 4-10 membered heterocyclyl optionally substituted with —(C═O)OC1-C6 alkyl,
(ii) 5-10 membered heteroaryl optionally substituted with 1-3 substituents independently selected from C1-C6 alkyl, hydroxyl, and C1-C6 hydroxyalkyl,
(iii) C3-C6 cycloalkyl optionally substituted with 1-2 substituents independently selected from C1-C6 alkyl and C1-C6 hydroxyalkyl,
(iv) ethylenyl,
(v) C1-C6 haloalkyl,
(vi) 4-10 membered heterocyclyl optionally substituted with —(C═O)OC1-C6 alkyl,
(vii) —NR^{1A}R^{1A}, or
(viii) phenyl;
R^{A} is hydrogen or C1-C6 alkyl;
R^{B} is
   (i) hydrogen,
   (ii) —S(O₂)C1-C6 alkyl,
   (iii) C3-C6 cycloalkyl optionally substituted with hydroxyl or C1-C6 alkoxy,
   (iv) —(C═O)C1-C6 alkyl,
   (v) —(C═O)OC1-C6 alkyl,
   (vi) 4-8 membered heterocyclyl optionally substituted with hydroxyl, or
   (vii) C1-C6 alkyl optionally substituted with 1-4 substituents independently selected from: halogen, hydroxyl, —NR^{C}R^{D}, C1-C6 alkoxy, C1-C6 haloalkoxy, C3-C6 cycloalkyl, phenyl optionally substituted with C1-C6 alkoxy, 5-6 membered heteroaryl optionally substituted with C1-C6 alkyl, and 4-8 membered heterocyclyl optionally substituted with —C(═O)C1-C6 alkyl or C1-C6 alkyl,
   (viii) —(C═O)5- or 6-membered heteroaryl optionally substituted with C1-C6 alkyl;
R² is
(i) hydrogen,
(ii) halogen,
(iii) C1-C6 alkoxy optionally substituted with 1-3 substituents independently selected from
   (a) hydroxyl,
   (b) halogen,
   (c) phosphate,
   (d) —NR^{2A}R^{2B},
   (e) 4-10 membered heterocyclyl optionally substituted with 1-3 substituents independently selected from hydroxyl, C1-C6 alkyl, C1-C6 haloalkyl, C1-C6 hydroxyalkyl, C1-C6 alkoxy, and C1-C6 alkoxyalkyl,
   (f) 5-6 membered heteroaryl optionally substituted with C1-C6 alkyl,
   (g) C3-C6 cycloalkyl optionally substituted with hydroxyl or hydroxyalkyl,
   (h) CO₂H,
   (i) C(O)R^{2A}
   (j) C1-C6 alkoxy; or
   (k) oxo;
(iv) C1-C6 haloalkoxy;
(v) 4-10 membered heterocyclyloxy,
(vi) —(C═O)NR^{2A}R^{2B},
(vii) C1-C6 alkyl optionally substituted with 1-3 substituents independently selected from hydroxyl, halogen, and —NR^{2A}R^{2B},
(viii) 5-6 membered heteroaryl optionally substituted with C1-C6 alkyl or C1-C6 alkoxy, or
(ix) —NR^{2A}R^{2B};
each R^{2A} and R^{2B} are independently hydrogen, C1-C6 alkyl optionally substituted with hydroxyl, or C1-C6 hydroxyalkyl;
R³ is
   (i) C1-C6 thioalkyl,

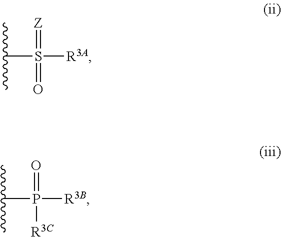

(iv) 4-8 membered heterocyclyl optionally substituted with 1-3 substituents independently selected from halogen, hydroxyl, C1-C6 alkyl, and C1-C6 alkoxy,
(v) C1-C6 alkyl optionally substituted with NR^{E}R^{F} or hydroxyl,
(vi) —CO₂H,
(vii) —C(═O)NR^{E}R^{F},
(viii) C1-C6 alkoxy optionally substituted with 4-10 membered heterocyclyl optionally substituted with C1-C6 alkoxy,
(ix) hydrogen,
(x) C1-C6 haloalkyl,
(xi) 5-6 membered heteroaryl optionally substituted with C1-C6 alkyl,
(xii) C1-C6 alkoxyalkyl, or
(xiii) C1-C6 hydroxyalkyl;
Z is O or NR⁴;
R^{3A} is
(i) C1-C6 haloalkyl,
(ii) C3-C6 cycloalkyl optionally substituted with C1-C6 alkyl,
(iii) C1-C6 alkyl optionally substituted with
   (a) C3-C6 cycloalkyl,
   (b) 5-6 membered heteroaryl optionally substituted with C1-C6 alkyl,
   (c) hydroxyl,
   (d) C1-C6 alkoxy, or
   (e) 4-6 membered heterocyclyl optionally substituted with 4-6
   membered heterocyclyl or C1-C6 alkyl optionally substituted with C1-C6 alkoxy,
(iv) C1-C6 alkoxyalkyl,
(v) C1-C6 hydroxyalkyl,
(vi) 5-6 membered heteroaryl optionally substituted with C1-C6 alkyl, or
(vii) 4-10 membered heterocyclyl optionally substituted with 1-3 substituents independently selected from hydroxyl, C1-C6 alkyl, —C(O)OC1-C6 alkyl, and C1-C6 alkoxy,
(viii) —N(C1-C6 alkyl)₂;
R^{3B} and R^{3C} are each independently C3-C6 cycloalkyl or C1-C6 alkyl optionally substituted with C3-C6 cycloalkyl, or
R^{3B} and R^{3C} with the atom to which they are attached together form a 4-8 membered heterocyclyl optionally substituted with C1-C6 alkyl;
R⁴ is hydrogen or C1-C6 alkyl; and
each R^{C} and R^{D} are each independently hydrogen, —(C═O)C1-C6 alkyl, or C1-C6 alkyl optionally substituted with oxo;
each R^{E} and R^{F} are each independently hydrogen or C1-C6 alkyl, or R^{E} and R^{F} with the atom to which they are attached together form a 4-8 membered heterocyclyl optionally substituted with hydroxyl.

In some embodiments, X is N. In some embodiments, X is C.

In some embodiments, Y is N. In some embodiments, Y is C.

In some embodiments, Z¹ is N. In some embodiments, Z¹ is C.

In some embodiments, one of X, Y, and Z¹ is N, and the other two of X, Y, and Z¹ are C. In some embodiments, two of X, Y, and Z¹ is N, and the other one of X, Y, and Z¹ are C.

In some embodiments, Ring A is 5-10 membered heteroaryl.

In some embodiments, Ring A is 5-6 membered heteroaryl. In some embodiments, Ring A is selected from the group consisting of pyrrolyl, pyrazolyl, imidazolyl, triazolyl, tetrazolyl, furanyl, thiophenyl, oxazolyl, isoxazolyl, isothiazolyl, thiazolyl, pyridinyl, pyrimidinyl, pyrazinyl, pyridazinyl, and pyridonyl.

In some embodiments, Ring A is 9-10 membered heteroaryl. In some embodiments, Ring A is indole, indazole, aza-indole, benzimidazole, benzothiophene, benzoxazole, benzothiazole, benzopyrazole, pyrrolopyrimidine, benzoisoxazole, benzotriazole, purine, quinoline, isoquinoline, quinazoline, quinoxaline, or cinnoline.

In some embodiments, Ring A is phenyl.

In some embodiments, R^{X} is hydrogen.

In some embodiments, R^{X} is halogen. In some embodiments, R^{X} is fluoro or chloro.

In some embodiments, R^{X} is —NH₂.

In the context of the R¹ group, “one or more” can refer to 1, 2, 3, 4, or any integer sub-range therein.

In some embodiments, one or more R¹ is C1-C6 alkoxy optionally substituted with hydroxyl or phenyl.

In some embodiments, one or more R¹ is C1-C6 alkoxyalkyl. In some embodiments, R¹ is C1-C6 alkoxyalkyl.

In some embodiments, one or more R¹ is C1-C6 deuteroalkoxy.

In some embodiments, one or more R¹ is 5-6 membered heteroaryl. In some embodiments, R¹ is 5-6 membered heteroaryl.

In some embodiments, one or more R¹ is —N(R^{1A})—S(O₂)R^{1B}. In some embodiments, R¹ is —N(R^{1A})—S(O₂)R^{1B}.

In some embodiments, one or more R¹ is —(C═O)NR^{1A}R^{1B}. In some embodiments, R¹ is —(C═O)NR^{1A}R^{1B}.

In some embodiments, one or more R¹ is phenyl.

In some embodiments, one or more R^{1A} is hydrogen. In some embodiments, R^{1A} is hydrogen.

In some embodiments, one or more R^{1A} is C1-C6 alkyl. In some embodiments, one or more R^{1A} is methyl. In some embodiments, R^{1A} is C1-C6 alkyl. In some embodiments, R^{1A} is methyl.

In some embodiments, one or more R^{1B} is C1-C6 alkyl optionally substituted with C3-C6 cycloalkyl or 4-10 membered heterocyclyl optionally substituted with —(C═O)OC1-C6 alkyl. In some embodiments, one or more R^{1B} is C1-C6 alkyl substituted with C3-C6 cycloalkyl or 4-10 membered heterocyclyl optionally substituted with —(C═O)OC1-C6 alkyl. In some embodiments, one or more R^{1B} is C1-C6 alkyl substituted with C3-C6 cycloalkyl. In some embodiments, one or more R^{1B} is C1-C6 alkyl substituted with 4-10 membered heterocyclyl optionally substituted with —(C═O)OC1-C6 alkyl. In some embodiments, R^{1B} is C1-C6 alkyl substituted with C3-C6 cycloalkyl or 4-10 membered heterocyclyl optionally substituted with —(C═O)OC1-C6 alkyl. In some embodiments, R^{1B} is C1-C6 alkyl substituted with C3-C6 cycloalkyl. In some embodiments, R^{1B} is C1-C6 alkyl substituted with 4-10 membered heterocyclyl optionally substituted with —(C═O)OC1-C6 alkyl.

In some embodiments, one or more R^{1B} is C1-C6 alkyl. In some embodiments, one or more R^{1B} is methyl. In some embodiments, R^{1B} is C1-C6 alkyl. In some embodiments, R^{1B} is methyl.

In some embodiments, one or more R^{1B} is a 5-10 membered heteroaryl. In some embodiments, one or more R^{1B} is a 5-6 membered heteroaryl. In some embodiments, R^{1B} is a 5-10 membered heteroaryl. In some embodiments, R^{1B} is a 5-6 membered heteroaryl.

In some embodiments, one or more R^{1B} is a 5-10 membered heteroaryl optionally substituted with 1-3 substituents independently selected from C1-C6 alkyl, hydroxyl, and C1-C6 hydroxyalkyl. In some embodiments, one or more R^{1B} is a 5-6 membered heteroaryl optionally substituted with 1-3 substituents independently selected from C1-C6 alkyl, hydroxyl, and C1-C6 hydroxyalkyl. In some embodiments, R^{1B} is a 5-10 membered heteroaryl substituted with 1-3 substituents independently selected from C1-C6 alkyl, hydroxyl, and C1-C6 hydroxyalkyl. In some embodiments, R^{1B} is a 5-6 membered heteroaryl substituted with 1-3 substituents independently selected from C1-C6 alkyl, hydroxyl, and C1-C6 hydroxyalkyl.

In some embodiments, one or more R^{1B} is a 5-10 membered heteroaryl optionally substituted with 1-3 substituents independently selected from C1—C6 alkyl, hydroxyl, and C1-C6 hydroxyalkyl. In some embodiments, R^{1B} is a 5-10 membered heteroaryl optionally substituted with 1-3 independently selected C1-C6 alkyl. In some embodiments, R^{1B} is a 5-6 membered heteroaryl optionally substituted with 1-3 independently selected C1-C6 alkyl. In some embodiments, R^{1B} is a 5-10 membered heteroaryl substituted with 1-3 independently selected C1-C6 alkyl. In some embodiments, R^{1B} is a 5-6 membered heteroaryl substituted with 1-3 independently selected C1-C6 alkyl. In some embodiments, R^{1B} is a 5-10 membered heteroaryl substituted with 1-3 independently substituents selected from C1-C6 alkyl and hydroxyl. In some embodiments, R^{1B} is a 5-6 membered heteroaryl substituted with 1-3 substituents independently selected from C1-C6 alkyl and hydroxyl. In some embodiments, R^{1B} is a 5-10 membered heteroaryl optionally substituted with 1-3 independently selected C1-C6 alkyl. In some embodiments, R^{1B} is a 5-10 membered heteroaryl optionally substituted with 1-3 independently selected C1-C6 alkyl. In some embodiments, R^{1B} is a 5-6 membered heteroaryl optionally substituted with 1-3 independently selected C1-C6 alkyl. In some embodiments, R^{1B} is a 5-10 membered heteroaryl substituted with 1-3 independently selected C1-C6 alkyl. In some embodiments, R^{1B} is a 5-6 membered heteroaryl substituted with 1-3 independently selected C1-C6 alkyl.

In some embodiments, one or more R^{1B} is a 5-10 membered heteroaryl optionally substituted with 1-2 substituents independently selected from C1-C6 alkyl, hydroxyl, and C1-C6 hydroxyalkyl. In some embodiments, one or more R^{1B} is a 5-6 membered heteroaryl optionally substituted with 1-2 substituents independently selected from C1-C6 alkyl, hydroxyl, and C1-C6 hydroxyalkyl. In some embodiments, R^{1B} is a 5-10 membered heteroaryl substituted with 1-2 substituents independently selected from C1-C6 alkyl, hydroxyl, and C1-C6 hydroxyalkyl. In some embodiments, R^{1B} is a 5-6 membered heteroaryl substituted with 1-2 substituents independently selected from C1-C6 alkyl, hydroxyl, and C1-C6 hydroxyalkyl.

In some embodiments, one or more R^{1B} is a 5-10 membered heteroaryl optionally substituted with 1-2 substituents independently selected from C1-C6 alkyl, hydroxyl, and C1-C6 hydroxyalkyl. In some embodiments, R^{1B} is a 5-10 membered heteroaryl optionally substituted with 1-2 independently selected C1-C6 alkyl. In some embodiments, R^{1B} is a 5-6 membered heteroaryl optionally substituted with 1-2 independently selected C1-C6 alkyl. In some embodiments, R^{1B} is a 5-10 membered heteroaryl substituted with 1-2 independently selected C1-C6 alkyl. In some embodiments, R^{1B} is a 5-6 membered heteroaryl substituted with 1-2 independently selected C1-C6 alkyl. In some embodiments, R^{1B} is a 5-10 membered heteroaryl substituted with 1-2 independently substituents selected from C1-C6 alkyl and hydroxyl. In some embodiments, R^{1B} is a 5-6 membered heteroaryl substituted with 1-2 substituents independently selected from C1-C6 alkyl and hydroxyl. In some embodiments, R^{1B} is a 5-10 membered heteroaryl optionally substituted with 1-2 independently selected C1-C6 alkyl. In some embodiments, R^{1B} is a 5-10 membered heteroaryl optionally substituted with 1-2 independently selected C1-C6 alkyl. In some embodiments, R^{1B} is a 5-6 membered heteroaryl optionally substituted with 1-2 independently selected C1-C6 alkyl. In some embodiments, R^{1B} is a 5-10 membered heteroaryl substituted with 1-2 independently selected C1-C6 alkyl. In some embodiments, R^{1B} is a 5-6 membered heteroaryl substituted with 1-2 independently selected C1-C6 alkyl.

In some embodiments, one or more R^{1B} is a 5-10 membered heteroaryl optionally substituted with C1-C6 alkyl, hydroxyl, or C1-C6 hydroxyalkyl. In some embodiments, one or more R^{1B} is a 5-6 membered heteroaryl optionally substituted with C1-C6 alkyl, hydroxyl, or C1-C6 hydroxyalkyl. In some embodiments, R^{1B} is a 5-10 membered heteroaryl substituted with C1-C6 alkyl, hydroxyl, or C1-C6 hydroxyalkyl. In some embodiments, R^{1B} is a 5-6 membered heteroaryl substituted with C1-C6 alkyl, hydroxyl, or C1-C6 hydroxyalkyl.

In some embodiments, R^{1B} is a 5-10 membered heteroaryl optionally substituted with C1-C6 alkyl. In some embodiments, R^{1B} is a 5-6 membered heteroaryl optionally substituted with C1-C6 alkyl. In some embodiments, R^{1B} is a 5-10 membered heteroaryl substituted with C1-C6 alkyl. In some embodiments, R^{1B} is a 5-6 membered heteroaryl substituted with C1-C6 alkyl. In some embodiments, R^{1B} is a 5-10 membered heteroaryl substituted with C1-C6 alkyl and hydroxyl. In some embodiments, R^{1B} is a 5-6 membered heteroaryl substituted with C1-C6 alkyl and hydroxyl.

In some embodiments, one or more R^{1B} is C3-C6 cycloalkyl optionally substituted with 1-2 substituents independently selected from C1-C6 alkyl and C1-C6 hydroxyalkyl. In some embodiments, one or more R^{1B} is C3-C6 cycloalkyl substituted with 1-2 substituents independently selected from C1-C6 alkyl and C1-C6 hydroxyalkyl. In some embodiments, one or more R^{1B} is C3-C6 cycloalkyl substituted with C1-C6 alkyl. In some embodiments, one or more R^{1B} is C3-C6 cycloalkyl substituted with C1-C6 hydroxyalkyl. In some embodiments R^{1B} is C3-C6 cycloalkyl optionally substituted with 1-2 substituents independently selected from C1-C6 alkyl and C1-C6 hydroxyalkyl. In some embodiments, R^{1B} is C3-C6 cycloalkyl substituted with 1-2 substituents independently selected from C1-C6 alkyl and C1-C6 hydroxyalkyl. In some embodiments, R^{1B} is C3-C6 cycloalkyl substituted with C1-C6 alkyl. In some embodiments, R^{1B} is C3-C6 cycloalkyl substituted with C1-C6 hydroxyalkyl. In some embodiments, one or more R^{1B} is C3-C6 cycloalkyl. In some embodiments, R^{1B} is C3-C6 cycloalkyl.

In some embodiments, one or more R^{1B} is ethylenyl.

In some embodiments, one or more R^{1B} is C1-C6 haloalkyl. In some embodiments, one or more R^{1B} is C1-C3 haloalkyl. In some embodiments, one or more R^{1B} is difluoromethyl or trifluoromethyl. In some embodiments, R^{1B} is C1-C6 haloalkyl. In some embodiments, R^{1B} is C1-C3 haloalkyl. In some embodiments, R^{1B} is difluoromethyl or trifluoromethyl.

In some embodiments, one or more R^{1B} is 4-10 membered heterocyclyl optionally substituted with —(C═O)OC1-C6 alkyl. In some embodiments, one or more R^{1B} is 4-10 membered heterocyclyl substituted with —(C═O)OC1-C6 alkyl. In some embodiments, one or more R^{1B} is 4-6 membered heterocyclyl substituted with —(C═O)OC1-C6 alkyl. In some embodiments, R^{1B} is 4-10 membered heterocyclyl optionally substituted with —(C═O)OC1-C6 alkyl. In some embodiments, R^{1B} is 4-10 membered heterocyclyl substituted with —(C═O)OC1-C6 alkyl. In some embodiments, R^{1B} is 4-6 membered heterocyclyl substituted with —(C═O)OC1-C6 alkyl. In some embodiments, R^{1B} is 4-10 membered heterocyclyl. In some embodiments, R^{1B} is 4-6 membered heterocyclyl.

In some embodiments, one or more R^{1B} is —NR^{1A}R^{1A}. In some embodiments, R^{1B} is —NR^{1A}R^{1A}.

In some embodiments, one or more R^{1B} is phenyl. In some embodiments, R^{1B} is phenyl.

In some embodiments, one or more R¹ is halogen. In some embodiments, Ri is fluoro or chloro.

In some embodiments, one or more Ri is cyano.

In some embodiments, one or more R¹ is hydroxyl.

In some embodiments, one or more R¹ is —NR^{A}R^{B}.

In some embodiments, one or more R¹ is C1-C6 alkyl.

In some embodiments, one or more R¹ is C1-C6 alkyl optionally substituted with 1-2 substituents independently selected from hydroxyl and 4-8 membered heterocyclyl optionally substituted with hydroxyl or C1-C6 alkyl.

In some embodiments, R^{A} is hydrogen.

In some embodiments, R^{A} is C1-C6 alkyl. In some embodiments, R^{A} is C1-C3 alkyl. In some embodiments, R^{A} is methyl.

In some embodiments, R^{B} is hydrogen.

In some embodiments, R^{B} is —S(O₂)C1-C6 alkyl. In some embodiments, R^{B} is —S(O₂)C1-C3 alkyl. In some embodiments, R^{B} is —S(O₂)CH₃.

In some embodiments, R^{B} is C3-C6 cycloalkyl optionally substituted with hydroxyl or C1-C6 alkoxy. In some embodiments, R^{B} is C3-C6 cycloalkyl substituted with hydroxyl or C1-C6 alkoxy. In some embodiments, R^{B} is C3-C6 cycloalkyl substituted with hydroxyl. In some embodiments, R^{B} is C3-C6 cycloalkyl substituted with C1-C6 alkoxy. In some embodiments, R^{B} is C3-C6 cycloalkyl.

In some embodiments, R^{B} is —(C═O)C1-C6 alkyl. In some embodiments, R^{B} is —(C═O)C1-C3 alkyl. In some embodiments, R^{B} is —(C═O)CH₃.

In some embodiments, R^{B} is —(C═O)OC1-C6 alkyl. In some embodiments, R^{B} is —(C═O)OC1-C3 alkyl. In some embodiments, R^{B} is —(C═O)OCH₃.

In some embodiments, R^{B} is 4-8 membered heterocyclyl optionally substituted with hydroxyl. In some embodiments, R^{B} is 4-8 membered heterocyclyl substituted with hydroxyl. In some embodiments, R^{B} is 4-8 membered heterocyclyl.

In some embodiments, R^{B} is C1-C6 alkyl optionally substituted with 1-4 substituents independently selected from: halogen, hydroxyl, —NR^{C}R^{D}, C1-C6 alkoxy, C1-C6 haloalkoxy, C3-C6 cycloalkyl, phenyl optionally substituted with C1-C6 alkoxy, 5-6 membered heteroaryl optionally substituted with C1-C6 alkyl, and 4-8 membered heterocyclyl optionally substituted with —C(═O)C1-C6 alkyl or C1-C6 alkyl.

In some embodiments, R^{B} is C1-C6 alkyl substituted with 1-4 substituents independently selected from: halogen, hydroxyl, —NR^{C}R^{D}, C1-C6 alkoxy, C1-C6 haloalkoxy, C3-C6 cycloalkyl, phenyl optionally substituted with C1-C6 alkoxy, 5-6 membered heteroaryl optionally substituted with C1-C6 alkyl, and 4-8 membered heterocyclyl optionally substituted with —C(═O)C1-C6 alkyl or C1-C6 alkyl.

In some embodiments, R^{B} is C1-C6 alkyl substituted with 3 substituents independently selected from: halogen, hydroxyl, —NR^{C}R^{D}, C1-C6 alkoxy, C1-C6 haloalkoxy, C3-C6 cycloalkyl, phenyl optionally substituted with C1-C6 alkoxy, 5-6 membered heteroaryl optionally substituted with C1-C6 alkyl, and 4-8 membered heterocyclyl optionally substituted with —C(═O)C1-C6 alkyl or C1-C6 alkyl.

In some embodiments, R^{B} is C1-C6 alkyl substituted with 1 or 2 substituents independently selected from: halogen, hydroxyl, —NR^{C}R^{D}, C1-C6 alkoxy, C1-C6 haloalkoxy, C3-C6 cycloalkyl, phenyl optionally substituted with C1-C6 alkoxy, 5-6 membered heteroaryl optionally substituted with C1-C6 alkyl, and 4-8 membered heterocyclyl optionally substituted with —C(═O)C1-C6 alkyl or C1-C6 alkyl.

In some embodiments, R^{B} is C1-C6 alkyl substituted with halogen, hydroxyl, —NR^{C}R^{D}, C1-C6 alkoxy, C1-C6 haloalkoxy, C3-C6 cycloalkyl, phenyl optionally substituted with C1-C6 alkoxy, 5-6 membered heteroaryl optionally substituted with C1-C6 alkyl, or 4-8 membered heterocyclyl optionally substituted with —C(═O)C1-C6 alkyl or C1-C6 alkyl.

In some embodiments, R^{B} is C1-C6 alkyl. In some embodiments, R^{B} is C1-C3 alkyl. In some embodiments, R^{B} is methyl.

In some embodiments, R^{B} is —(C═O)5- or 6-membered heteroaryl optionally substituted with C1-C6 alkyl.

In some embodiments, R^{A} and R^{B} are the same. In some embodiments, R^{A} and R^{B} are different. In some embodiments, R^{A} and R^{B} are each methyl. In some embodiments, R^{A} and R^{B} are each hydrogen.

In some embodiments, one or more R¹ is C1-C6 alkyl optionally substituted with hydroxyl or 4-8 membered heterocyclyl optionally substituted with hydroxyl. In some embodiments, one or more R¹ is C1-C6 alkyl substituted with hydroxyl or 4-8 membered heterocyclyl optionally substituted with hydroxyl. In some embodiments, R¹ is C1-C6 alkyl optionally substituted with hydroxyl or 4-8 membered heterocyclyl optionally substituted with hydroxyl. In some embodiments, R¹ is C1-C6 alkyl substituted with hydroxyl or 4-8 membered heterocyclyl optionally substituted with hydroxyl. In some embodiments, R¹ is C1-C6 alkyl substituted with hydroxyl. In some embodiments, R¹ is C1-C6 alkyl substituted with 4-8 membered heterocyclyl optionally substituted with hydroxyl. In some embodiments, R¹ is C1-C6 alkyl. In some embodiments, R¹ is C1-C3 alkyl. In some embodiments, R¹ is methyl.

In some embodiments, one or more R¹ is C1-C6 haloalkyl. In some embodiments, one or more R¹ is C1-C3 haloalkyl. In some embodiments, one or more R¹ is difluoromethyl or trifluoromethyl. In some embodiments, R¹ is C1-C6 haloalkyl. In some embodiments, R¹ is C1-C3 haloalkyl. In some embodiments, R¹ is difluoromethyl or trifluoromethyl.

In some embodiments, one or more R¹ is C1-C6 haloalkoxy. In some embodiments, one or more R¹ is C1-C3 haloalkoxy. In some embodiments, one or more R¹ is difluoromethoxy or trifluoromethoxy. In some embodiments, R¹ is C1-C6 haloalkoxy. In some embodiments, R¹ is C1-C3 haloalkoxy. In some embodiments, R¹ is difluoromethoxy or trifluoromethoxy.

In some embodiments, one or more R¹ is C3-C6 cycloalkyl. In some embodiments, R¹ is C3-C6 cycloalkyl.

In some embodiments, one or more R¹ is 4-8 membered heterocyclyl optionally substituted with 1-2 substituents independently selected from hydroxyl, C1-C6 alkyl, C1-C6 haloalkyl- and —(C═O)OC1-C6 alkyl. In some embodiments, one or more R¹ is 4-8 membered heterocyclyl optionally substituted with hydroxyl, C1-C6 alkyl, C1-C6 haloalkyl, —(C═O)OC1-C6 alkyl. In some embodiments, one or more R¹ is 4-8 membered heterocyclyl optionally substituted with 2 substituents independently selected from the group consisting of hydroxyl, C1-C6 alkyl, C1-C6 haloalkyl, —(C═O)OC1-C6 alkyl.

In some embodiments, R¹ is 4-8 membered heterocyclyl optionally substituted with 1-2 substituents independently selected from the group consisting of hydroxyl, C1-C6 alkyl, C1-C6 haloalkyl, —(C═O)OC1-C6 alkyl. In some embodiments, R¹ is 4-8 membered heterocyclyl optionally substituted with hydroxyl, C1-C6 alkyl, C1-C6 haloalkyl, —(C═O)OC1-C6 alkyl. In some embodiments, R¹ is 4-8 membered heterocyclyl optionally substituted with 2 substituents independently selected from the group consisting of hydroxyl, C1-C6 alkyl, C1-C6 haloalkyl, —(C═O)OC1-C6 alkyl.

In some embodiments, R¹ is 4-8 membered heterocyclyl substituted with hydroxyl. In some embodiments, R¹ is 4-8 membered heterocyclyl substituted with C1-C6 alkyl. In some embodiments, R¹ is 4-8 membered heterocyclyl substituted with C1-C6 haloalkyl. In some embodiments, R¹ is 4-8 membered heterocyclyl substituted—with —(C═O)OC1-C6 alkyl. In some embodiments, R¹ is 4-8 membered heterocyclyl.

In some embodiments, one or more R¹ is —S(O₂)C1-C6 alkyl. In some embodiments, one or more R¹ is —S(O₂)C1-C3 alkyl. In some embodiments, one or more R¹ is —S(O₂)CH₃. In some embodiments, R¹ is —S(O₂)C1-C6 alkyl. In some embodiments, R¹ is —S(O₂)C1-C3 alkyl. In some embodiments, R¹ is —S(O₂)CH₃.

In some embodiments, one or more R¹ is 4-10 membered heterocyclyloxy optionally substituted with acyl. In some embodiments, one or more R¹ is 4-10 membered heterocyclyloxy substituted with acyl. In some embodiments, one or more R¹ is 4-10 membered heterocyclyloxy. In some embodiments, R¹ is 4-10 membered heterocyclyloxy optionally substituted with acyl. In some embodiments, R¹ is 4-10 membered heterocyclyloxy substituted with acyl. In some embodiments, R¹ is 4-10 membered heterocyclyloxy.

In some embodiments, m is 0 or 1. In some embodiments, m is 1 or 2. In some embodiments, m is 2 or 3. In some embodiments, m is 3 or 4.

In some embodiments, m is 0.

In some embodiments, m is 1.

In some embodiments, m is 2.

In some embodiments, m is 3.

In some embodiments, m is 4.

In some embodiments, R² is hydrogen.

In some embodiments, R² is halogen. In some embodiments, R² is chloro or fluoro.

In some embodiments, R² is C1-C6 alkoxy optionally substituted with 1-3 substituents independently selected from
(a) hydroxyl,
(b) haloI,
(c) phosphate,
(d) —NR^{2A}R^{2B},
(e) 4-10 membered heterocyclyl optionally substituted with 1-3 substituents independently selected from hydroxyl, C1-C6 alkyl, C1-C6 haloalkyl, C1-C6 hydroxyalkyl, C1-C6 alkoxy, and C1-C6 alkoxyalkyl,
(f) 5-6 membered heteroaryl optionally substituted with C1-C6 alkyl, and
(g) C3-C6 cycloalkyl optionally substituted with hydroxyl or hydroxyalkyl,
(h) CO₂H,
(i) C(O)R^{2A},
(j) C1-C6 alkoxy; or
(k) oxo.

In some embodiments, R² is C1-C6 alkoxy substituted with 1 or 2 substituents independently selected from
(a) hydroxyl,
(b) hIgen,
(c) phosphate,
(d) —NR^{2A}R^{2B},
(e) 4-10 membered heterocyclyl optionally substituted with 1-3 substituents independently selected from hydroxyl, C1-C6 alkyl, C1-C6 haloalkyl, C1-C6 hydroxyalkyl, C1-C6 alkoxy, and C1-C6 alkoxyalkyl,
(f) 5-6 membered heteroaryl optionally substituted with C1-C6 alkyl, and
(g) C3-C6 cycloalkyl optionally substituted with hydroxyl.

In some embodiments, R² is C1-C6 alkoxy substituted with 3 substituents independently selected from
(a) hydroxyl, (bIalogen,
(c) phosphate,
(d) —NR^{2A}R^{2B},
(e) 4-10 membered heterocyclyl optionally substituted with 1-3 substituents independently selected from hydroxyl, C1-C6 alkyl, C1-C6 haloalkyl, C1-C6 hydroxyalkyl, C1-C6 alkoxy, and C1-C6 alkoxyalkyl,
(f) 5-6 membered heteroaryl optionally substituted with C1-C6 alkyl, and
(g) C3-C6 cycloalkyl optionally substituted with hydroxyl.

In some embodiments, R² is C1-C6 alkoxy substituted with hydroxyl. In some embodiments, R² is C1-C6 alkoxy substituted with hydroxyl and one or two independently selected halogen. In some embodiments, R² is C1-C6 alkoxy substituted with 1-3 independently selected halogen.

In some embodiments, R² is C1-C6 alkoxy substituted with phosphate. In some embodiments, R² is C1-C6 alkoxy substituted with NR^{2A}R^{2B}. In some embodiments, R² is C1-C6 alkoxy substituted with 4-10 membered heterocyclyl optionally substituted with 1-3 substituents independently selected from hydroxyl, C1-C6 alkyl, C1-C6 haloalkyl, C1-C6 hydroxyalkyl, C1-C6 alkoxy, and C1-C6 alkoxyalkyl. In some embodiments, R² is C1-C6 alkoxy substituted with 4-10 membered heterocyclyl substituted with 1-3 substituents independently selected from hydroxyl, C1-C6 alkyl, C1-C6 haloalkyl, C1-C6 hydroxyalkyl, C1-C6 alkoxy, and C1-C6 alkoxyalkyl. In some embodiments, R² is C1-C6 alkoxy substituted with 5-6 membered heteroaryl optionally substituted with C1-C6 alkyl. In some embodiments, R² is C1-C6 alkoxy substituted with 5-6 membered heteroaryl substituted with C1-C6 alkyl. In some embodiments, R² is C1-C6 alkoxy substituted with 5-6 membered heteroaryl. In some embodiments, R² is C1-C6 alkoxy substituted with C3-C6 cycloalkyl optionally substituted with hydroxyl. In some embodiments, R² is C1-C6 alkoxy substituted with C3-C6 cycloalkyl substituted with hydroxyl. In some embodiments, R² is C1-C6 alkoxy substituted with C3-C6 cycloalkyl.

In some embodiments, R² is C1-C6 alkoxy. In some embodiments, R² is C1-C3 alkoxy. In some embodiments, R² is methoxy.

In some embodiments, R² is C1-C6 haloalkoxy. In some embodiments, R² is C1-C3 haloalkoxy. In some embodiments, R² is difluoromethoxy or trifluoromethoxy.

In some embodiments, R² is 4-10 membered heterocyclyloxy. In some embodiments, R² is 4-6 membered heterocyclyloxy. In some embodiments, R² is 7-10 membered heterocyclyloxy.

In some embodiments, R² is —(C═O)NR^{2A}R^{2B}.

In some embodiments, R² is C1-C6 alkyl optionally substituted with 1-3 substituents independently selected from hydroxyl, halogen, and —NR^{2A}R^{2B}. In some embodiments, R² is C1-C6 alkyl substituted with halogen. In some embodiments, R² is C1-C6 alkyl substituted with NR^{2A}R^{2B}. In some embodiments, R² is C1-C6 alkyl substituted with hydroxyl. In some embodiments, R² is hydroxyethyl. In some embodiments, R² is C1-C6 alkyl. In some embodiments, R² is C1-C3 alkyl. In some embodiments, R² is methyl.

In some embodiments, R² is 5-6 membered heteroaryl optionally substituted with C1-C6 alkyl or C1-C6 alkoxy. In some embodiments, R² is 5-6 membered heteroaryl substituted with C1-C6 alkyl or C1-C6 alkoxy. In some embodiments, R² is 5-6 membered heteroaryl substituted with C1-C6 alkyl. In some embodiments, R² is 5-6 membered heteroaryl substituted with C1-C6 alkoxy. In some embodiments, R² is 5-6 membered heteroaryl.

In some embodiments, R² is NR^{2A}R^{2B}. In some embodiments, R^{2A} and R^{2B} are hydrogen.

In some embodiments, each R^{2A} and R^{2B} are independently hydrogen, C1-C6 alkyl optionally substituted with hydroxyl, or C1-C6 hydroxyalkyl.

In some embodiments, one or more R^{2A} is hydrogen.

In some embodiments, one or more R^{2A} is C1-C6 alkyl. In some embodiments, R^{2A} is C1-C3 alkyl. In some embodiments, R^{2A} is methyl.

In some embodiments, one or more R^{2A} is C1-C6 hydroxyalkyl. In some embodiments, one or more R^{2A} is C1-C3 hydroxyalkyl. In some embodiments, one or more R^{2A} is hydroxyethyl.

In some embodiments, one or more R^{2B} is hydrogen.

In some embodiments, one or more R^{2B} is C1-C6 alkyl. In some embodiments, R^{2B} is C1-C3 alkyl. In some embodiments, R^{2B} is methyl.

In some embodiments, one or more R^{2B} is C1-C6 hydroxyalkyl. In some embodiments, one or more R^{2B} is C1-C3 hydroxyalkyl. In some embodiments, one or more R^{2B} is hydroxyethyl. In some embodiments, R^{2B} is C1-C6 hydroxyalkyl. In some embodiments, R^{2B} is C1-C3 hydroxyalkyl. In some embodiments, R^{2B} is hydroxyethyl.

In some embodiments, R² is selected from the group consisting of: H, OMe,

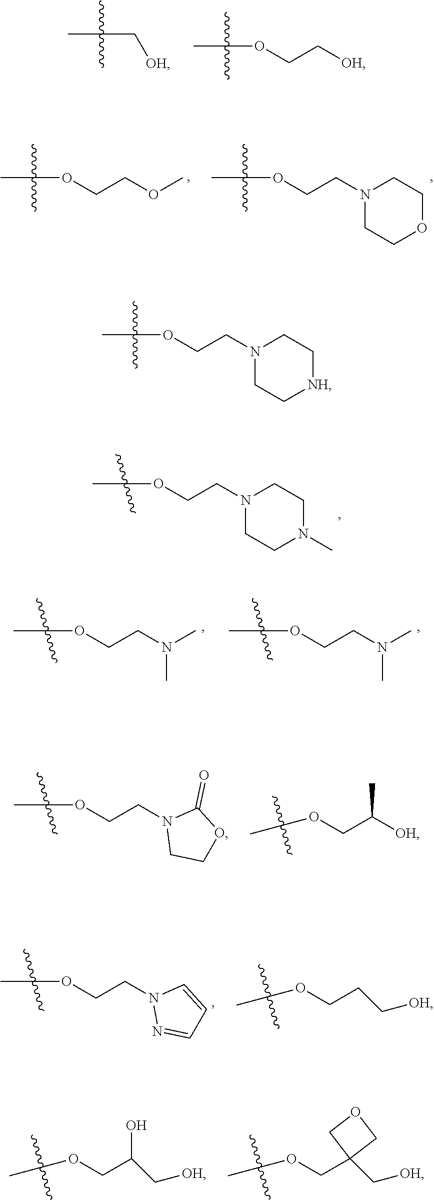

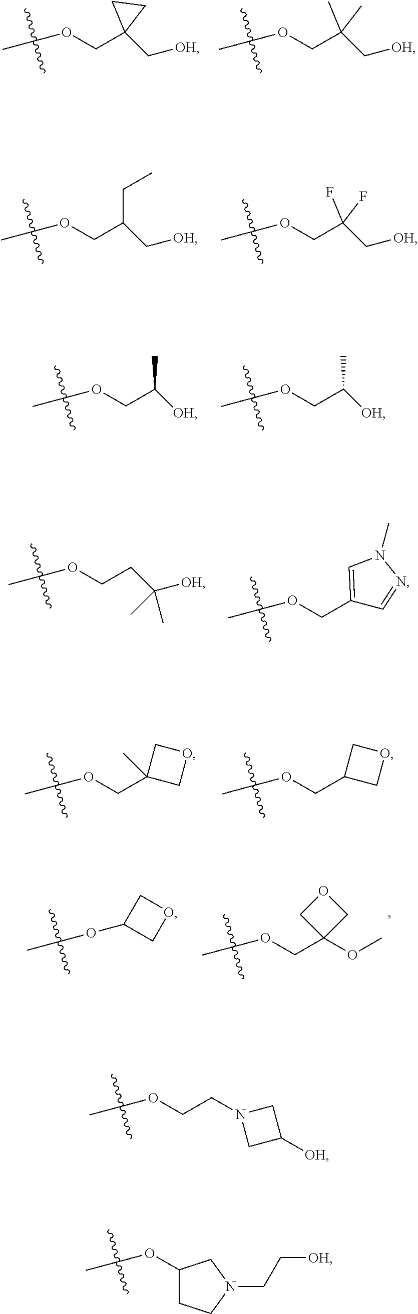

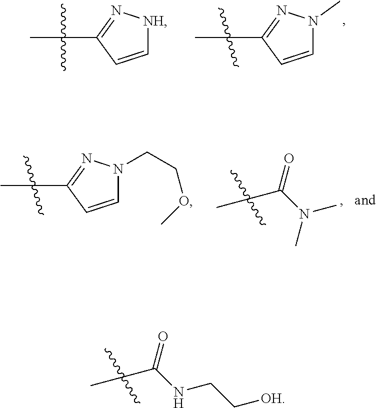

In some embodiments, R³ is C1-C6 thioalkyl.

In some embodiments, R³ is —CO₂H.

In some embodiments, R³ is C1-C6 alkoxy optionally substituted with 4-10 membered heterocyclyl optionally substituted with C1-C6 alkoxy. In some embodiments, R³ is C1-C6 alkoxy substituted with 4-10 membered heterocyclyl optionally substituted with C1-C6 alkoxy. In some embodiments, R³ is C1-C6 alkoxy optionally substituted with 4-6 membered heterocyclyl optionally substituted with C1-C6 alkoxy. In some embodiments, R³ is C1-C6 alkoxy optionally substituted with 4-10 membered heterocyclyl substituted with C1-C6 alkoxy. In some embodiments, R³ is C1-C6 alkoxy.

In some embodiments, R³ is 4-8 membered heterocyclyl optionally substituted with 1-3 substituents independently selected from halogen, hydroxyl, C1-C6 alkyl, oxo, and C1-C6 alkoxy. In some embodiments, R³ is 4-8 membered heterocyclyl substituted with 1-3 substituents independently selected from halogen, hydroxyl, C1-C6 alkyl, and C1-C6 alkoxy. In some embodiments, R³ is 4-8 membered heterocyclyl optionally substituted with 1 or 2 substituents independently selected from halogen, hydroxyl, C1-C6 alkyl, and C1-C6 alkoxy. In some embodiments, R³ is 4-8 membered heterocyclyl optionally substituted with halogen, hydroxyl, C1-C6 alkyl, or C1-C6 alkoxy. In some embodiments, R³ is 4-8 membered heterocyclyl.

In some embodiments, R³ is hydrogen.

In some embodiments, R³ is oxo.

In some embodiments, R³ is C1-C6 haloalkyl. In some embodiments, R³ is C1-C3 haloalkyl. In some embodiments, R³ is difluoromethyl or trifluoromethyl.

In some embodiments, R³ is 5-6 membered heteroaryl optionally substituted with C1-C6 alkyl. In some embodiments, R³ is 5-6 membered heteroaryl substituted with C1-C6 alkyl. In some embodiments, R³ is 5-6 membered heteroaryl substituted with methyl. In some embodiments, R³ is 5-6 membered heteroaryl.

In some embodiments, R³ is C1-C6 alkoxyalkyl. In some embodiments, R³ is C1-C3 alkoxyalkyl. In some embodiments, R³ is methoxyethyl or methoxypropyl.

In some embodiments, R³ is C1-C6 hydroxyalkyl. In some embodiments, R³ is C1-C3 hydroxyalkyl. In some embodiments, R³ is hydroxyethyl.

In some embodiments, R³ is C1-C6 alkyl optionally substituted with NR^{E}R^{F} or hydroxyl. In some embodiments, R³ is C1-C6 alkyl substituted with NR^{E}R^{F} or hydroxyl. In some embodiments, R³ is C1-C6 alkyl substituted with NR^{E}R^{F}. In some embodiments, R³ is C1-C6 alkyl substituted with hydroxyl. In some embodiments, R³ is C1-C6 alkyl. In some embodiments, R³ is C1-C3 alkyl. In some embodiments, R³ is methyl.

In some embodiments, R³ is —C(═O)NR^{E}R^{F}.

In some embodiments, R^{E} is hydrogen.

In some embodiments, R^{E} is C1-C6 alkyl. In some embodiments, R^{E} is C1-C3 alkyl. In some embodiments, R^{E} is methyl.

In some embodiments, R^{F} is hydrogen.

In some embodiments, R^{F} is C1-C6 alkyl. In some embodiments, R^{F} is C1-C3 alkyl. In some embodiments, R^{F} is methyl.

In some embodiments, R^{E} and R^{F} are the same. In some embodiments, R^{E} and R^{F} are different. In some embodiments, R^{E} and R^{F} are each methyl. In some embodiments, R^{E} and R^{F} are each hydrogen.

In some embodiments, R^{E} and R^{F} with the atom to which they are attached together form a 4-8 membered heterocyclyl optionally substituted with hydroxyl. In some embodiments, R^{E} and R^{F} with the atom to which they are attached together form a 4-8 membered heterocyclyl substituted with hydroxyl. In some embodiments, R^{E} and R^{F} with the atom to which they are attached together form a 4-8 membered heterocyclyl.

In some embodiments, R³ is

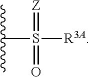

In some embodiments, R^{3A} is C1-C6 haloalkyl. In some embodiments, R^{3A} is C1-C3 haloalkyl. In some embodiments, R^{3A} is difluoromethyl or trifluoromethyl.

In some embodiments, R^{3A} is C3-C6 cycloalkyl optionally substituted with C1-C6 alkyl. In some embodiments, R^{3A} is C3-C6 cycloalkyl substituted with C1-C6 alkyl. In some embodiments, R^{3A} is C3-C6 cycloalkyl substituted with methyl. In some embodiments, R^{3A} is C3-C6 cycloalkyl.

In some embodiments, R^{3A} is C1-C6 alkyl optionally substituted with
(a) C3-C6 cycloalkyl,
(b) 5-6 membered heteroaryl optionally substituted with C1-C6 alkyl,
(c) hydroxyl,
(d) C1-C6 alkoxy, or
(e) 4-6 membered heterocyclyl optionally substituted with 4-6 membered heterocyclyl or C1-C6 alkyl optionally substituted with C1-C6 alkoxy.

In some embodiments, R^{3A} is C1-C6 alkyl substituted with
(a) C3-C6 cycloalkyl,
(b) 5-6 membered heteroaryl optionally substituted with C1-C6 alkyl,
(c) hydroxyl,
(d) C1-C6 alkoxy, or
(e) 4-6 membered heterocyclyl optionally substituted with 4-6 membered heterocyclyl or C1-C6 alkyl optionally substituted with C1-C6 alkoxy.

In some embodiments, R^{3A} is C1-C6 alkyl substituted with C3-C6 cycloalkyl. In some embodiments, R^{3A} is C1-C6 alkyl substituted with 5-6 membered heteroaryl optionally substituted with C1-C6 alkyl. In some embodiments, R^{3A} is C1-C6 alkyl substituted with 5-6 membered heteroaryl substituted with C1-C6 alkyl. In some embodiments, R^{3A} is C1-C6 alkyl substituted with 5-6 membered heteroaryl. In some embodiments, R^{3A} is C1-C6 alkyl substituted with 4-6 membered heterocyclyl optionally substituted with C1-C6 alkyl. In some embodiments, R^{3A} is C1-C6 alkyl substituted with 4-6 membered heterocyclyl substituted with C1-C6 alkyl. In some embodiments, R^{3A} is C1-C6 alkyl substituted with 4-6 membered heterocyclyl. In some embodiments, R^{3A} is C1-C6 alkyl. In some embodiments, R^{3A} is C1-C3 alkyl. In some embodiments, R^{3A} is methyl.

In some embodiments, R^{3A} is C1-C6 alkoxyalkyl. In some embodiments, R^{3A} is C1-C3 alkoxyalkyl. In some embodiments, R^{3A} is methoxyethyl or methoxypropyl.

In some embodiments, R^{3A} is C1-C6 hydroxyalkyl. In some embodiments, R^{3A} is C1-C3 hydroxyalkyl. In some embodiments, R^{3A} is hydroxyethyl.

In some embodiments, R^{3A} is 5-6 membered heteroaryl optionally substituted with C1-C6 alkyl. In some embodiments, R^{3A} is 5-6 membered heteroaryl substituted with C1-C6 alkyl. In some embodiments, R^{3A} is 5-6 membered heteroaryl substituted with methyl. In some embodiments, R^{3A} is 5-6 membered heteroaryl.

In some embodiments, R^{3A} is 4-10 membered heterocyclyl optionally substituted with 1-3 substituents independently selected from hydroxyl, C1-C6 alkyl, and C1-C6 alkoxy. In some embodiments, R^{3A} is 4-10 membered heterocyclyl substituted with 1-3 substituents independently selected from hydroxyl, C1-C6 alkyl, and C1-C6 alkoxy. In some embodiments, R^{3A} is 4-10 membered heterocyclyl substituted with 1 or 2 substituents independently selected from hydroxyl, C1-C6 alkyl, and C1-C6 alkoxy. In some embodiments, R^{3A} is 4-10 membered heterocyclyl substituted with 1 hydroxyl, C1-C6 alkyl, or C1-C6 alkoxy. In some embodiments, R^{3A} is 4-10 membered heterocyclyl.

In some embodiments, R³ is selected from the group consisting of: H, OMe, CF₃, OH

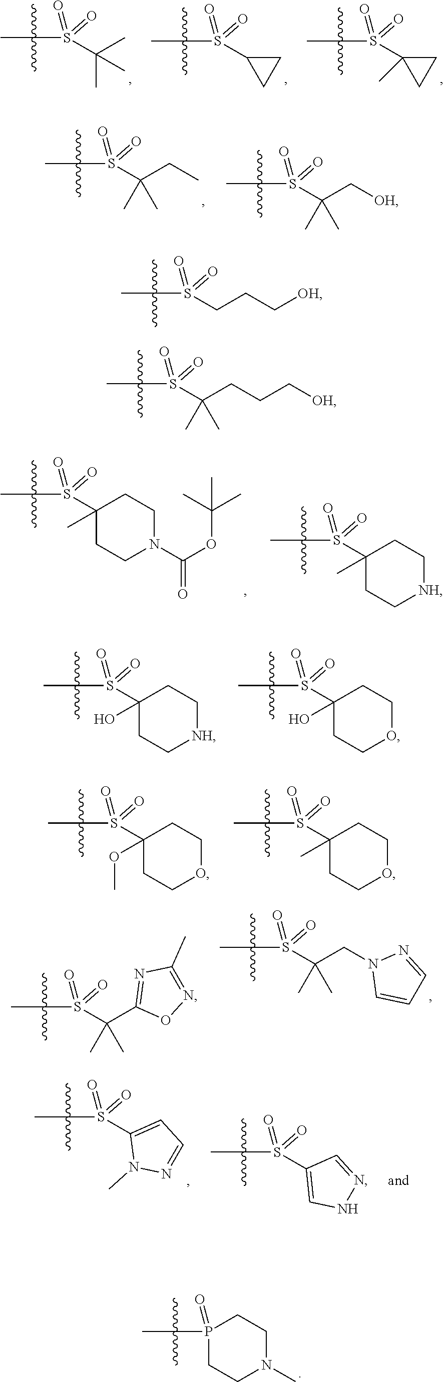

In some embodiments, Z is NR⁴.

In some embodiments, R⁴ is hydrogen.

In some embodiments, R⁴ is C1-C6 alkyl. In some embodiments, R⁴ is C1-C3 alkyl. In some embodiments, R⁴ is methyl.

In some embodiments, Z is O.

In some embodiments, R³ is

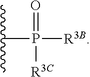

In some embodiments, R^{3B} is C3-C6 cycloalkyl. In some embodiments, R^{3B} is cyclopropyl or cyclobutyl.

In some embodiments, R^{3B} is C1-C6 alkyl optionally substituted with C3-C6 cycloalkyl. In some embodiments, R^{3B} is C1-C6 alkyl substituted with C3-C6 cycloalkyl. In some embodiments, R^{3B} is methyl substituted with C3-C6 cycloalkyl. In some embodiments, R^{3B} is C1-C6 alkyl. In some embodiments, R^{3B} is C1-C3 alkyl. In some embodiments, R^{3B} is methyl.

In some embodiments, R^{3C} is C3-C6 cycloalkyl.

In some embodiments, R^{3C} is or C1-C6 alkyl optionally substituted with C3-C6 cycloalkyl. In some embodiments, R^{3C} is C1-C6 alkyl substituted with C3-C6 cycloalkyl. In some embodiments, R^{3C} is methyl substituted with C3-C6 cycloalkyl. In some embodiments, R^{3C} is C1-C6 alkyl. In some embodiments, R^{3C} is C1-C3 alkyl. In some embodiments, R^{3C} is methyl.

In some embodiments, R^{3B} and R^{3C} are the same. In some embodiments, R^{3B} and R^{3C} are different. In some embodiments, R^{3B} and R^{3C} are each methyl.

In some embodiments, R^{3B} and R^{3C} with the atom to which they are attached together form a 4-8 membered heterocyclyl optionally substituted with C1-C6 alkyl.

In some embodiments, R^{C} is hydrogen.

In some embodiments, R^{C} is C1-C6 alkyl optionally substituted with oxo. In some embodiments, R^{C} is C1-C6 alkyl substituted with oxo. In some embodiments, R^{C} is acyl. In some embodiments, R^{C} is C1-C6 alkyl. In some embodiments, R^{C} is C1-C3 alkyl. In some embodiments, R^{C} is methyl.

In some embodiments, R^{D} is hydrogen.

In some embodiments, R^{D} is C1-C6 alkyl optionally substituted with oxo. In some embodiments, R^{D} is C1-C6 alkyl substituted with oxo. In some embodiments, R^{D} is acyl. In some embodiments, R^{D} is C1-C6 alkyl. In some embodiments, R^{D} is C1-C3 alkyl. In some embodiments, R^{D} is methyl.

In some embodiments, Formula (I) is (I-a):

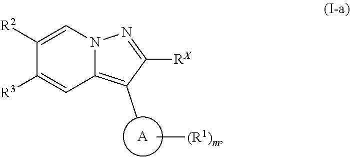

or a pharmaceutically acceptable salt thereof.

In some embodiments, Formula (I) is (I-b):

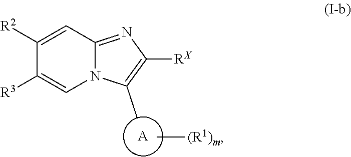

or a pharmaceutically acceptable salt thereof.

In some embodiments, Formula (I) is (I-c):

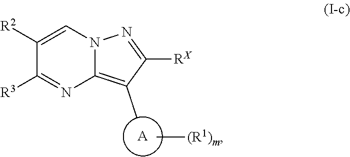

or a pharmaceutically acceptable salt thereof.

In some embodiments, Formula (I) is (I-d):

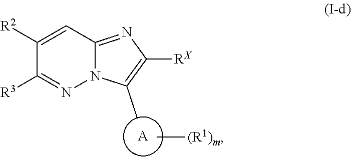

or a pharmaceutically acceptable salt thereof.

In some embodiments, Formula (I) is (I-e):

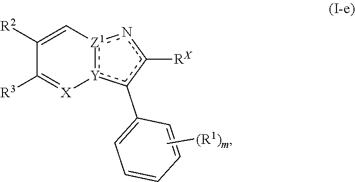

or a pharmaceutically acceptable salt thereof.

In some embodiments, Formula (I) is (I-f):

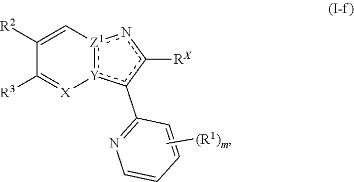

or a pharmaceutically acceptable salt thereof.

In some embodiments, Formula (I) is (I-g):

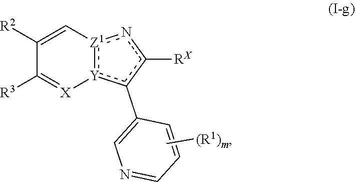

or a pharmaceutically acceptable salt thereof.

In some embodiments, Formula (I) is (I-h):

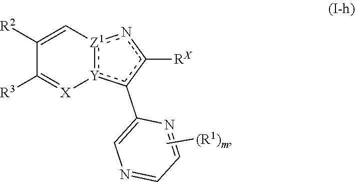

or a pharmaceutically acceptable salt thereof.

In some embodiments, Formula (I) is (I-i):

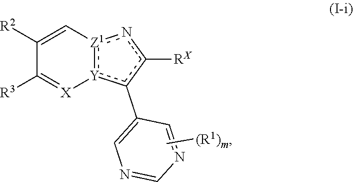

or a pharmaceutically acceptable salt thereof.

In some embodiments, Formula (I) is (I-j):

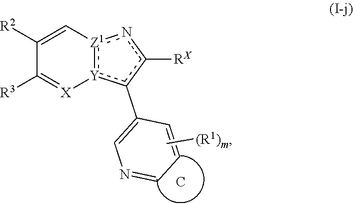

or a pharmaceutically acceptable salt thereof, wherein ring C is morpholine, N-methylmorpholine, pyrrolidinone, imidazole, or pyrrole. In some embodiments, ring C is morpholine. In some embodiments, ring C is N-methylmorpholine. In some embodiments, ring C is pyrrolidinone. In some embodiments, ring C is imidazole. In some embodiments, ring C is pyrrole.

In some embodiments, Formula (I) is (I-k):

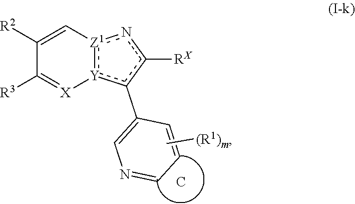

or a pharmaceutically acceptable salt thereof, wherein ring C is pyridine or pyrimidine. In some embodiments, ring C is pyridine. In some embodiments, ring C is pyrimidine.

In some embodiments, Formula (I) is (I-1):

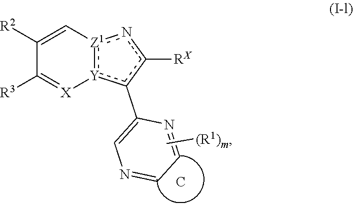

or a pharmaceutically acceptable salt thereof, wherein ring C is pyridine or pyrimidine. In some embodiments, ring C is pyridine. In some embodiments, ring C is pyrimidine.

In some embodiments, Formula (I) is (I-m):

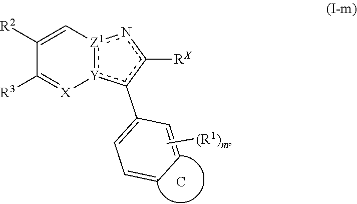

or a pharmaceutically acceptable salt thereof, wherein ring C is morpholine, N-methylmorpholine, pyrrolidinone, imidazole, or pyrrole. In some embodiments, ring C is morpholine. In some embodiments, ring C is N-methylmorpholine. In some embodiments, ring C is pyrrolidinone. In some embodiments, ring C is imidazole. In some embodiments, ring C is pyrrole.

In some embodiments, Formula (I) is (I-j):

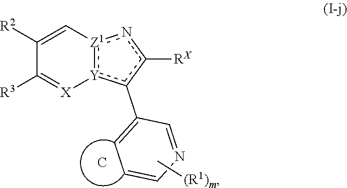

or a pharmaceutically acceptable salt thereof, wherein ring C is morpholine, N-methylmorpholine, pyrrolidinone, imidazole, or pyrrole. In some embodiments, ring C is morpholine. In some embodiments, ring C is N-methylmorpholine. In some embodiments, ring C is pyrrolidinone. In some embodiments, ring C is imidazole. In some embodiments, ring C is pyrrole.

In some embodiments, Formula (I) is (I-k):

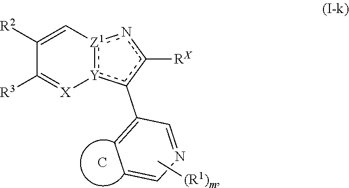

or a pharmaceutically acceptable salt thereof, wherein ring C is pyridine or pyrimidine. In some embodiments, ring C is pyridine. In some embodiments, ring C is pyrimidine.

In some embodiments, Formula (I) is (I-l):

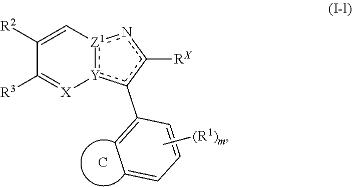

or a pharmaceutically acceptable salt thereof, wherein ring C is pyridine or pyrimidine. In some embodiments, ring C is pyridine. In some embodiments, ring C is pyrimidine.

In some embodiments, Formula (I) is (I-m):

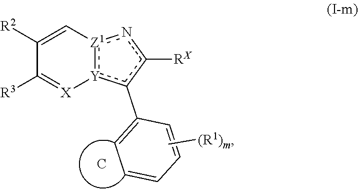

or a pharmaceutically acceptable salt thereof, wherein ring C is morpholine, N-methylmorpholine, pyrrolidinone, imidazole, or pyrrole. In some embodiments, ring C is morpholine. In some embodiments, ring C is N-methylmorpholine. In some embodiments, ring C is pyrrolidinone. In some embodiments, ring C is imidazole. In some embodiments, ring C is pyrrole.

In some embodiments, Formula (I) is (I-n):

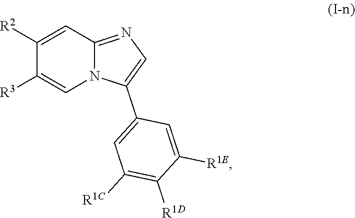

or a pharmaceutically acceptable salt thereof, wherein R^{1C}, R^{1D}, and R^{1E} are each an independently selected R¹.

In some embodiments, Formula (I) is (I-o):

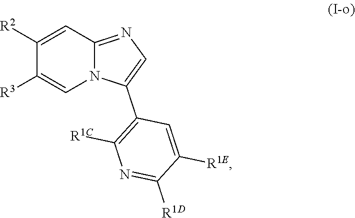

or a pharmaceutically acceptable salt thereof, wherein R^{1C}, R^{1D}, and R^{1E} are each an independently selected R¹.

In some embodiments, Formula (I) is (I-p):

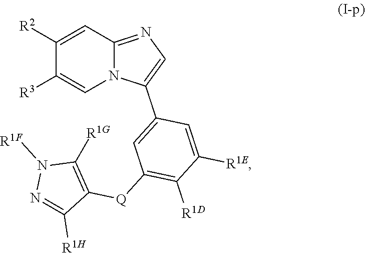

or a pharmaceutically acceptable salt thereof, wherein:
   R^{1D} and R^{1E} are each an independently selected R¹;
   Q is —NH(SO₂)— or —NH(C1-C6 alkyl)-; and
   R^{1F}, R^{1G}, and R^{1H} are each independently hydrogen, C1-C6 alkyl, or C1-C6 hydroxyalkyl, wherein at least one of R^{1F}, R^{1G}, and R^{1H} is hydrogen.

In some embodiments of Formula (I-p), R^{1D} is fluoro or methoxy and R^{1E} is fluoro or methoxy. In some embodiments of Formula (I-p), R^{1D} is methoxy and R^{1E} is fluoro or methoxy. In some embodiments of Formula (I-p), R^{1D} is methoxy and R^{1E} is fluoro.

In some embodiments of Formula (I-p), R^{1F} is hydrogen or methyl and R^{1G} and R^{1H} are each hydrogen. In some embodiments of Formula (I-p), R^{1F} is methyl.

In some embodiments of Formula (I-p), Q is —NH(SO₂)—, wherein the nitrogen atom is connected to the phenyl ring of Formula (I-p) and the sulfur atom is attached to the pyrazole ring of Formula (I-p).

In some embodiments of Formula (I-p), Q is —NH(C1-C6 alkyl)-, wherein the nitrogen atom is connected to the phenyl ring of Formula (I-p) and a carbon atom is attached to the pyrazole ring of Formula (I-p).

In some embodiments,

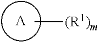

is selected from the group consisting of:

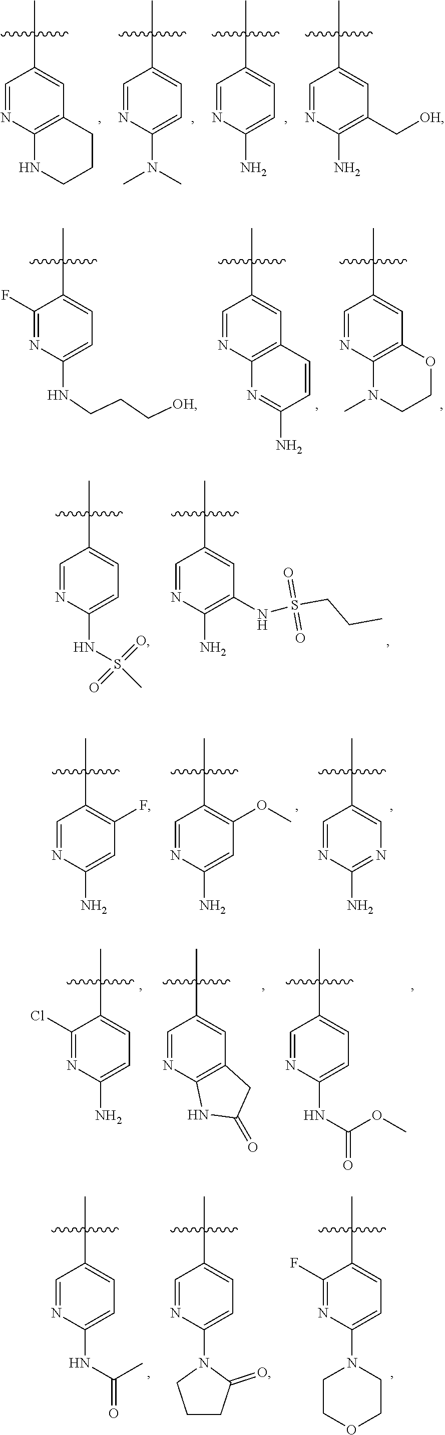

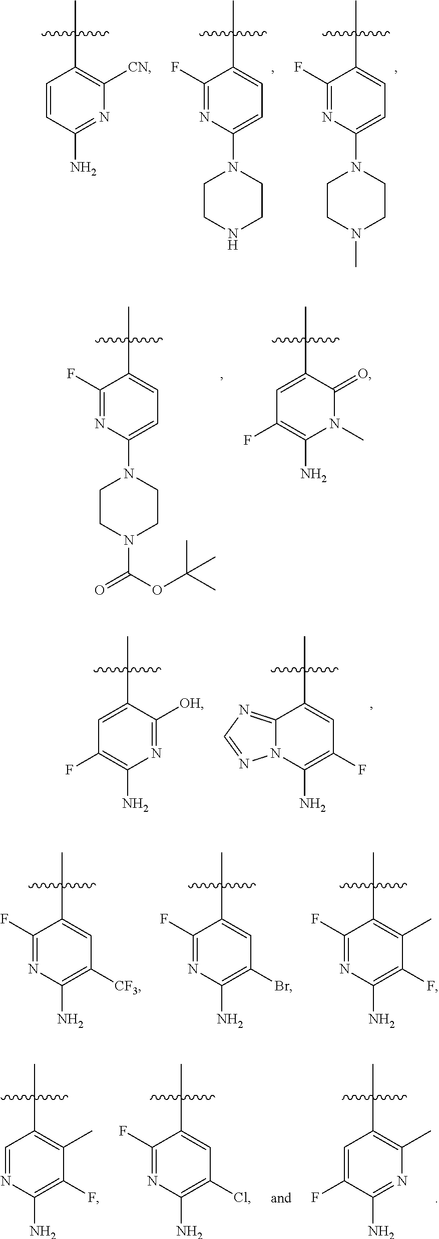

In some embodiments,

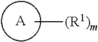

is selected from the group consisting of:

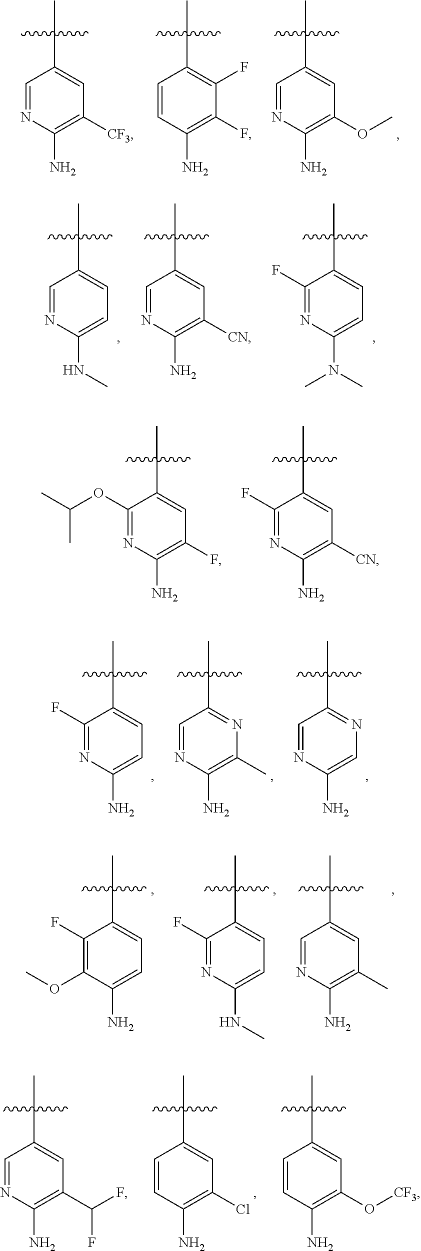

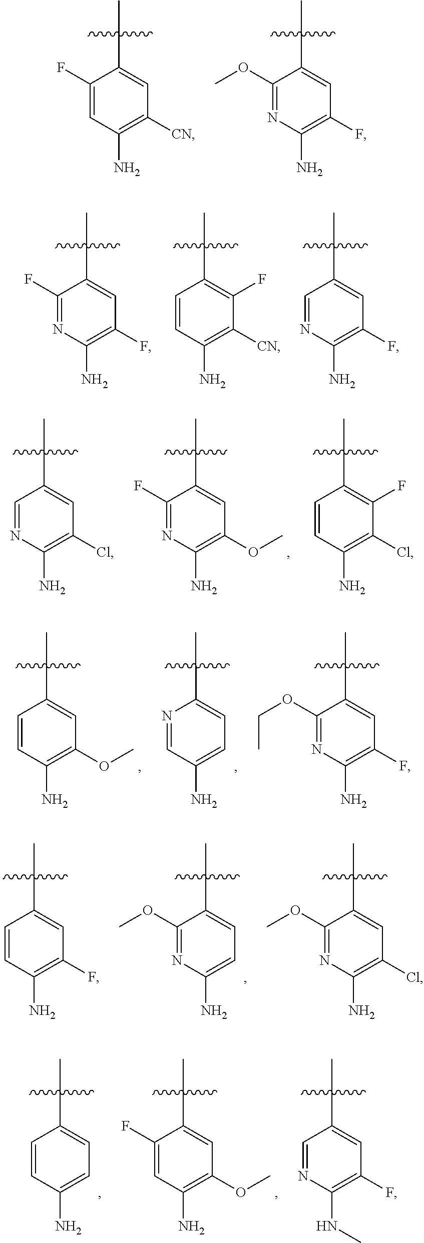

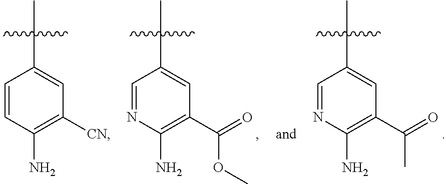

In some embodiments,

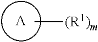

its selected from the group consisting of:

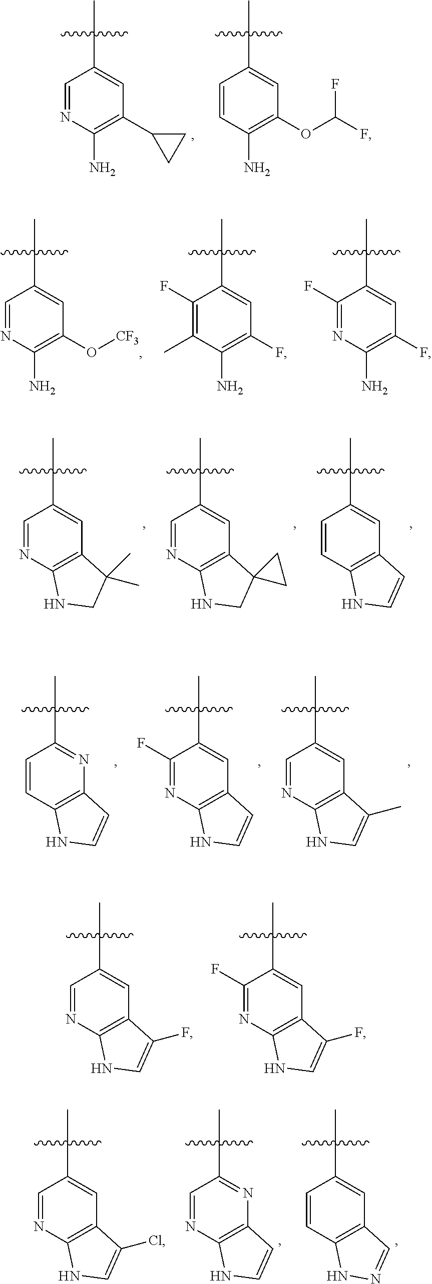

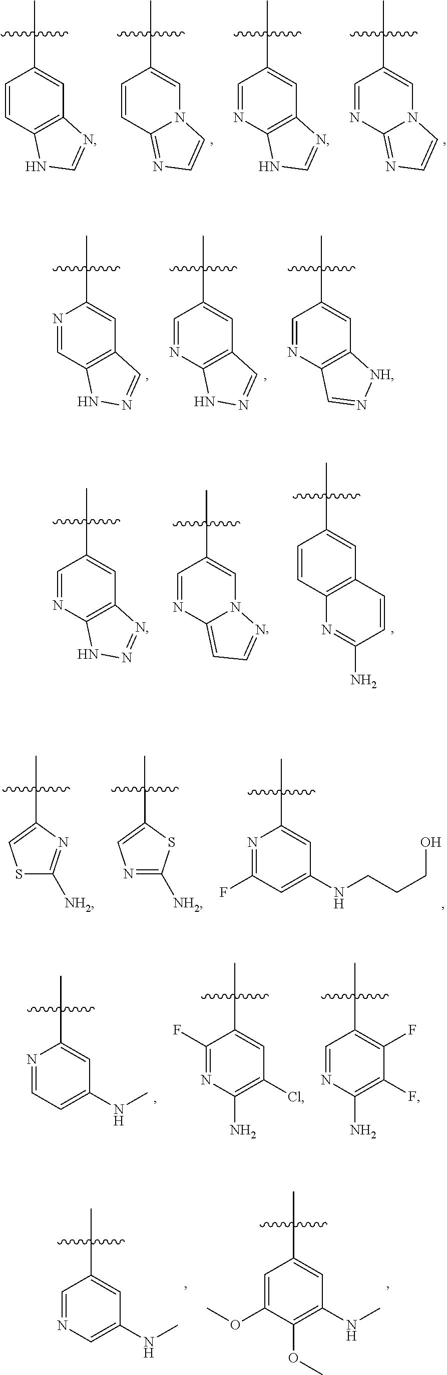

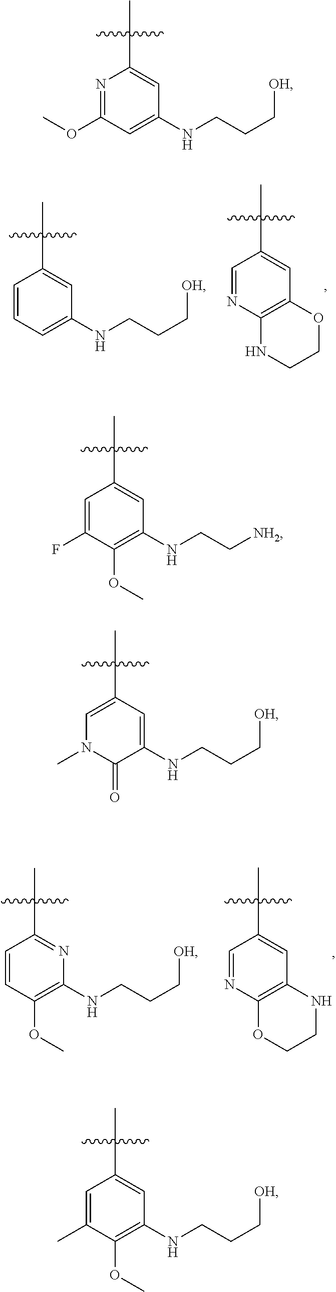

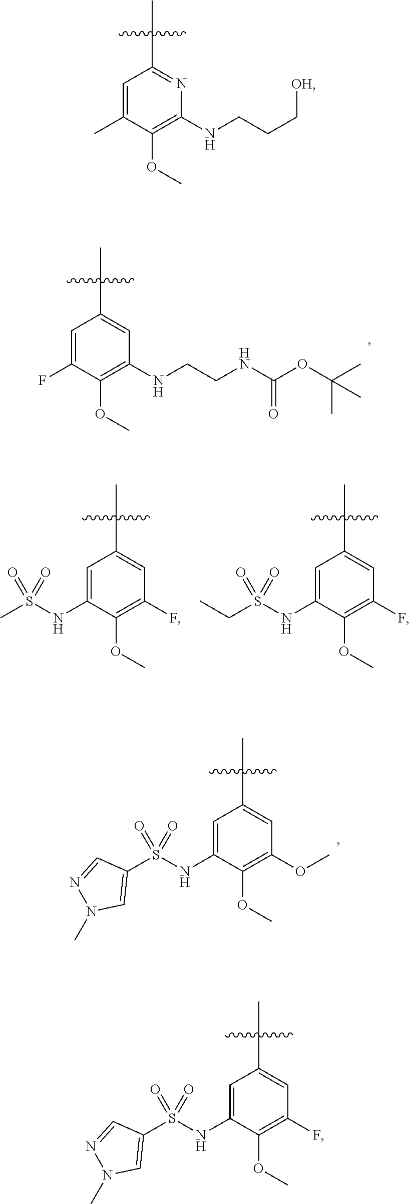

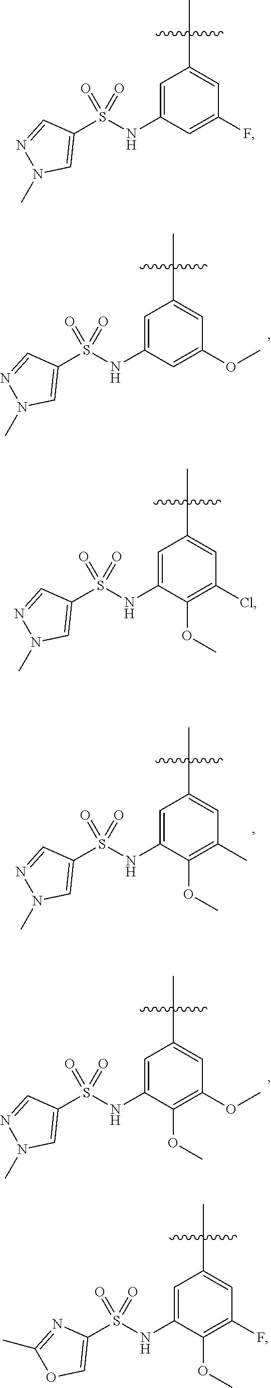

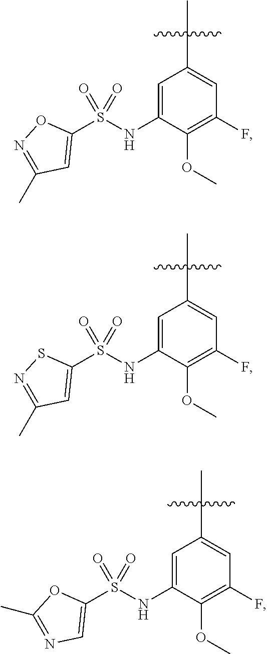 [SMILES: *c1cc(F)c(OC)c(NS(=O)(=O)c2cc(C)no2)c1 ; *c1cc(F)c(OC)c(NS(=O)(=O)c2cc(C)ns2)c1 ; *c1cc(F)c(OC)c(NS(=O)(=O)c2cnc(C)o2)c1]

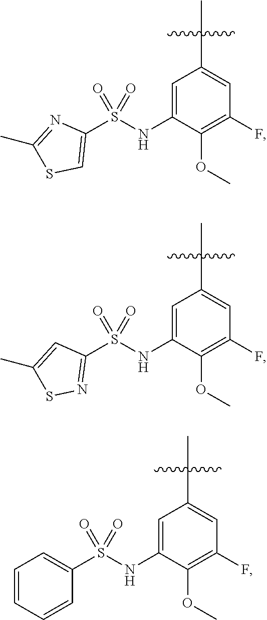 [SMILES: *c1cc(F)c(OC)c(NS(=O)(=O)c2csc(C)n2)c1 ; *c1cc(F)c(OC)c(NS(=O)(=O)c2cc(C)sn2)c1 ; *c1cc(F)c(OC)c(NS(=O)(=O)c2ccccc2)c1]

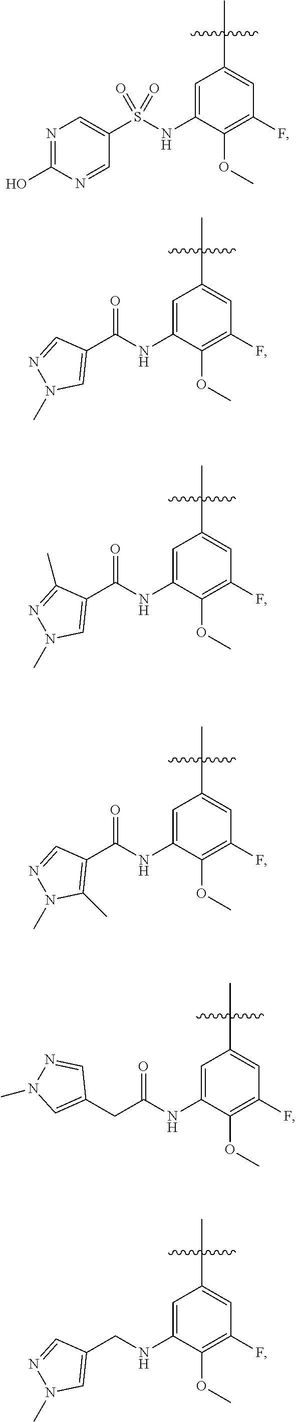 [SMILES: *c1cc(F)c(OC)c(NS(=O)(=O)c2cnc(O)nc2)c1 ; *c1cc(F)c(OC)c(NC(=O)c2cnn(C)c2)c1 ; *c1cc(F)c(OC)c(NC(=O)c2cn(C)nc2C)c1 ; *c1cc(F)c(OC)c(NC(=O)c2cnn(C)c2C)c1 ; *c1cc(F)c(OC)c(NC(=O)Cc2cnn(C)c2)c1 ; *c1cc(F)c(OC)c(NCc2cnn(C)c2)c1]

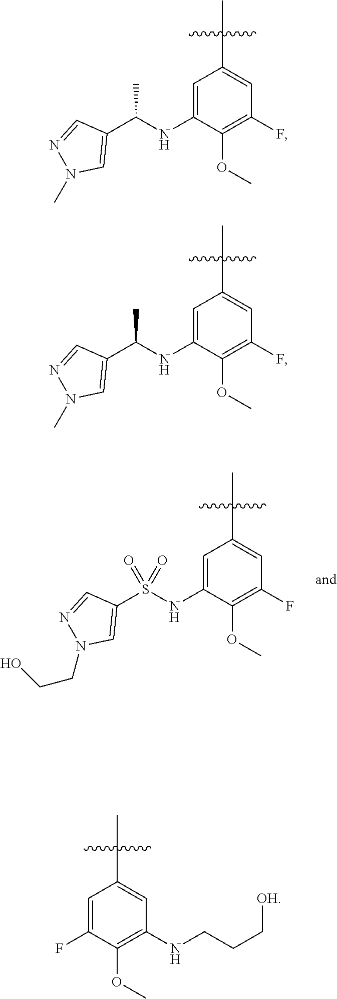 [SMILES: *c1cc(F)c(OC)c(N[C@@H](C)c2cnn(C)c2)c1 ; *c1cc(F)c(OC)c(N[C@H](C)c2cnn(C)c2)c1 ; *c1cc(F)c(OC)c(NS(=O)(=O)c2cnn(CCO)c2)c1 ; *c1cc(F)c(OC)c(NCCCO)c1]

In some embodiments

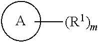

is selected from the group consisting of:

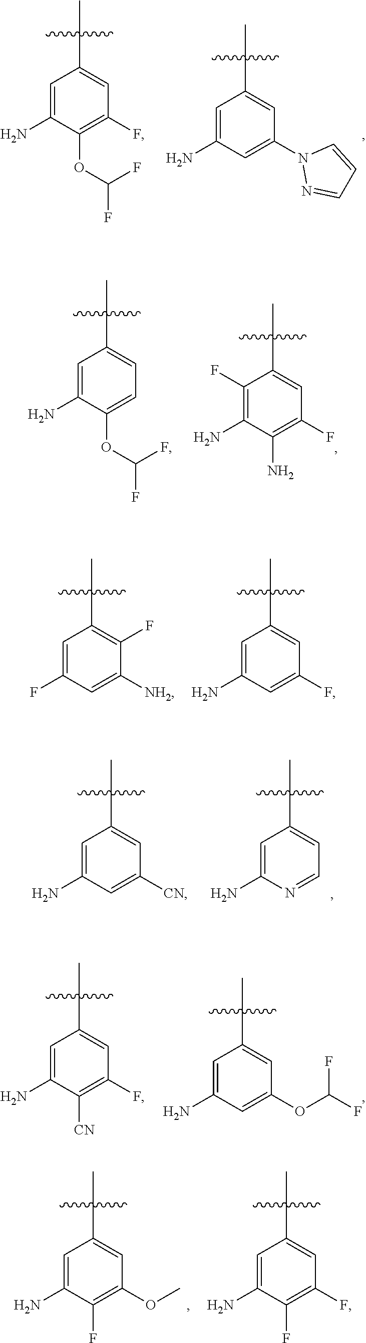

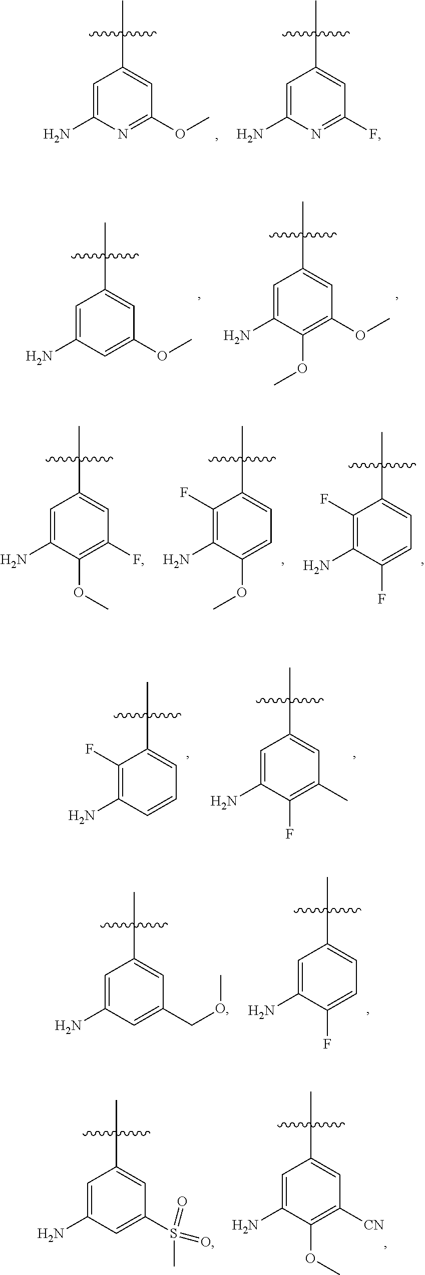

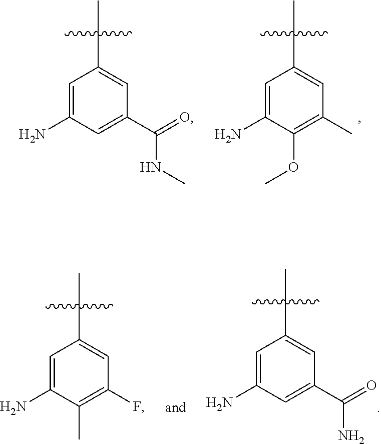

In some embodiments,

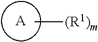

is selected from the group consisting of:

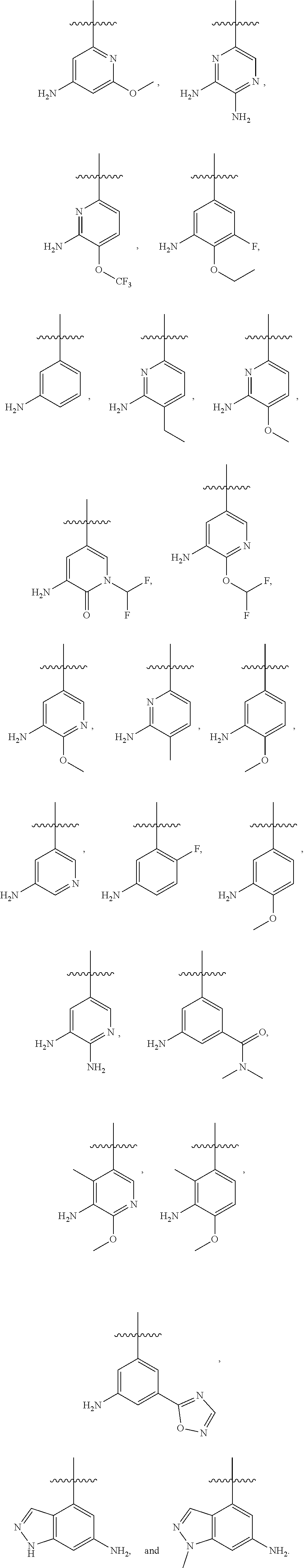

In some embodiments, the compounds of Formula (I), include the compounds of Examples 1-304 and pharmaceutically acceptable salts thereof. In some embodiments, the compounds of Examples 1-304 are in the free base form. In some embodiments, the compounds of Examples 1-304 are in salt form, e.g., pharmaceutically acceptable salt form.

The compounds of Formula (I), and pharmaceutically acceptable salts thereof, do not include the compounds exemplified in PCT Appl. No. PCT/US23/24988, or pharmaceutically acceptable salts thereof.

The ability of test compounds to act as RIPK2 inhibitors may be demonstrated by the biological assays described herein. See, e.g., Tables A1 and A.

### Pharmaceutical Compositions

Some embodiments provide a pharmaceutical composition comprising a compound of Formula (I), or a pharmaceutically acceptable salt thereof, and at least one pharmaceutically acceptable excipient.

### Methods of Treatment

The compounds and compositions disclosed herein are effective for modulating the activity of RIPK2. In some embodiments, the compounds and compositions disclosed herein are RIPK2 inhibitors.

The term “RIPK2-associated disease or disorder” as used herein refers to diseases or disorders associated with or having a dysregulation of a RIPK2 gene, a RIPK2 protein, or the expression or activity or level of any (e.g., one or more) of the same (e.g., any of the types of dysregulation of a RIPK2 gene, a RIPK2 protein, a RIPK2 protein domain, or the expression or activity or level of any of the same described herein).

An exemplary sequence of human RIPK2 is shown below (UniParc Accession No. UPI00001338F2):

MNGEAICSALPTIPYHKLADLRYLSRGASGTVSSARHADWRVQVAVK
HLHIHTPLLDSERKDVLREAEILHKARFSYILPILGICNEPEFLGI
VTEYMPNGSLNELLHRKTEYPDVAWPLRFRILHEIALGVNYLHNMT
PPLLHHDLKTQNILLDNEFHVKIADFGLSKWRMMSLSQSRSSKSAP
EGGTIIYMPPENYEPGQKSRASIKHDIYSYAVITWEVLSRKQPFED
VTNPLQIMYSVSQGHRPVINEESLPYDIPHRARMISLIESGWAQNP
DERPSFLKCLIELEPVLRTFEEITFLEAVIQLKKTKLQSVSSAIHL
CDKKKMELSLNIPVNHGPQEESCGSSQLHENSGSPETSRSLPAPQD
NDFLSRKAQDCYFMKLHHCPGNHSWDSTISGSQRAAFCDHKTTPCS
SAIINPLSTAGNSERLQPGIAQQWIQSKREDIVNQMTEACLNQSLD
ALLSRDLIMKEDYELVSTKPTRTSKVRQLLDTTDIQGEEFAKVIVQ
KLKDNKQMGLQPYPEILVVSRSPSLNLLQNKSM

Some embodiments provide a method of treating a RIPK2-associated disease or disorder in a subject in need thereof, comprising administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating a RIPK2-associated disease or disorder in a subject in need thereof, comprising (a) determining or having determined that the subject is suffering from a RIPK2-associated disease or disorder; and (b) administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating a RIPK2-associated disease or disorder in a subject in need thereof, comprising (a) determining or having determined that the subject has a RIPK2-associated disease or disorder; and (b) administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating a RIPK2-associated disease or disorder in a subject previously identified or diagnosed as having a RIPK2-associated disease or disorder, the method comprising administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

In some embodiments, the RIPK2-associated disease or disorder is a cardiovascular disease, an allergic disorder, an autoimmune disease, an inflammatory disease, a cardiovascular disease, a fibrotic disease, or a disease associated with abnormal cell growth (such as cancer).

In some embodiments, the RIPK2-associated disease or disorder is a cardiovascular disease.

In some embodiments, the RIPK2-associated disease or disorder is an allergic disorder.

In some embodiments, the RIPK2-associated disease or disorder is an autoimmune disease.

In some embodiments, the RIPK2-associated disease or disorder is an inflammatory disease.

In some embodiments, the RIPK2-associated disease or disorder is a cardiovascular disease.

In some embodiments, the RIPK2-associated disease or disorder is a fibrotic disease.

In some embodiments, the RIPK2-associated disease or disorder is a disease associated with abnormal cell growth (such as cancer). In some embodiments, the RIPK2-associated disease or disorder is a cancer.

In some embodiments, the RIPK2-associated disease or disorder is a Type I hypersensitivity (allergic) reaction. In some embodiments, the Type I hypersensitivity (allergic) reaction is allergic inflammation. In some embodiments, the allergic inflammation is allergic rhinitis, allergic asthma, allergic conjunctivitis, atopic- and vernal keratoconjunctivitis, or atopic dermatitis.

In some embodiments, the RIPK2-associated disease or disorder is an autoimmune disease. In some embodiments, the autoimmune disease is Crohn's disease, ulcerative colitis, rheumatoid arthritis, multiple sclerosis, encephalomyelitis, systemic lupus erythematosus, psoriasis, lupus nephritis, immune thrombocytopenic purpura, Sjogren's syndrome, ankylosing spondylitis, psoriatic arthritis, juvenile dermatomyositis, juvenile rheumatoid arthritis, juvenile spondyloarthopathy, non-radiographic spondyloarthopathy, Behcet's disease, dermatomyositis, diabetes mellitus type 1, Goodpasture's syndrome, Graves' disease, Guillain-Barre syndrome, Hashimoto's disease, mixed connective tissue damage, myasthenia gravis, narcolepsy, pemphigus vulgaris, pernicious anemia, polymyositis, primary biliary cirrhosis, temporal arteritis, or vasculitis. In some embodiments, the autoimmune disease is Crohn's disease, ulcerative colitis, inflammatory bowel disease, or multiple sclerosis. In some embodiments, the autoimmune disease is Crohn's disease. In some embodiments, the autoimmune disease is ulcerative colitis. In some embodiments, the autoimmune disease is inflammatory bowel disease. In some embodiments, the autoimmune disease is multiple sclerosis.

In some embodiments, the RIPK2-associated disease or disorder is a metabolic disease. In some embodiments, the metabolic disease is dysglycemia, type 2 diabetes, non-alcoholic fatty liver disease (including non-alcoholic steatohepatitis), or obesity.

In some embodiments, the RIPK2-associated disease or disorder is an inflammatory disease. In some embodiments, the inflammatory disease is chronic lung inflammatory disease, osteoarthritis, inflammatory arthritis, asthma, early onset sarcoidosis, sarcoidosis, eczema, allergic eczema, uveitis, reactive arthritis, chronic inflammation, chronic prostatitis, inflammatory bowel disease, glomerulonephritis, bursitis, carpal tunnel syndrome, tendinitis, inflammation of the lung (e.g., chronic obstructive pulmonary disease), pelvic inflammatory disease, transplant rejection, vasculitis, regional enteritis, distal ileitis, regional ileitis, and terminal ileitis, central areolar choroidal dystrophy, macular degeneration, retinosis pigmentosa, adult vitelliform disease, pattern dystrophy, diabetic retinopathy, BEST disease, myopic degeneration, central serous retinopathy, Stargardt's disease, Cone-Rod dystrophy, North Carolina dystrophy, infectious retinitis, inflammatory retinitis, uveitis, toxic retinitis, or systemic inflammatory response syndrome. In some embodiments, the inflammatory disease is inflammatory bowel disease.

In some embodiments, the RIPK2-associated disease or disorder isgranulomatous inflammatory disease. In some embodiments, the granulomatous inflammatory disease is Wegener's granulomatosis, Churg-Strauss syndrome, relapsing polychondritis, polyarteritis nodosa, giant cell arteritis, primary biliary cirrhosis, hepatic granulomato's disease, Langerhan's granulomatosis, granulomatous enteritis, orofacial granulomatosis, or Peyronie's disease.

In some embodiments, the RIPK2-associated disease or disorder is a cardiovascular disease. In some embodiments, the cardiovascular disease is atherosclerosis, thrombosis, myocardial infarction, stroke, aortic aneurysm, arterial hypertension, sickle cell crisis, or ischemia-reperfusion injury.

In some embodiments, the RIPK2-associated disease is lethal systemic inflammatory response syndrome, chronic gut and skin inflammation, or acute pancreatitis.

In some embodiments, the RIPK2-associated disease or disorder is a fibrotic disease. In some embodiments, the fibrotic disease is scleroderma, asbestosis, or idiopathic pulmonary fibrosis.

In some embodiments, the RIPK2-associated disease or disorder comprises neuroinflammation. In some embodiments, the RIPK2-associated disease or disorder is Alzheimer's disease, amyotrophic lateral sclerosis (ALS), Parkinson's disease, Huntington's disease, Lewy body disease, Niemann-Pick disease, type C1 (NPC1), Friedreich's ataxia, spinal muscular atrophy, corticobasal degeneration, progress supranuclear palsy (PSP), or multiple system atrophy (MSA).

In some embodiments, the RIPK2-associated disease or disorder is a disease related to abnormal cell growth. In some embodiments, the disease related to abnormal cell growth is cancer, including hematological malignancies and solid tumors.

Hematological malignancies include, but are not limited to leukemias, such as acute myeloid leukemia, chronic myelogenous leukemia, chronic lymphocytic leukemia, B-cell chronic lymphocytic leukemia, and lymphomas and myelomas, such as B-cell lymphoma (e.g., mantle cell lymphoma), T-cell lymphoma (e.g., peripheral T-cell lymphoma), non-Hodgkin's lymphoma, and multiple myeloma.

Solid tumors include lung cancer (small cell lung cancer and non-small cell lung cancer), pancreatic cancer, colon cancer, breast cancer, genitourinary cancer, skin cancer, bone cancer, prostate cancer, liver cancer, brain cancer, laryngeal cancer, gall bladder cancer, rectal cancer, parathyroid cancer, thyroid cancer, adrenal cancer, neural tissue cancer, bladder cancer, head and neck cancer, stomach cancer, gastric cancer, bronchial cancer, and kidney cancer (e.g., renal clear cell carcinoma), colorectal cancer, clear cell carcinoma, basal cell carcinoma, squamous cell carcinoma, esophageal cancer, metastatic skin carcinoma, osteosarcoma, Ewing's sarcoma, reticulum cell sarcoma, Kaposi's sarcoma, giant cell tumor, islet cell tumor, acute and chronic lymphocytic and granulocytic tumors, hairy-cell tumor, adenoma, medullary carcinoma, pheochromocytoma, mucosal neuromas, intestinal ganglioneuromas, hyperplastic corneal nerve tumor, marfanoid habitus tumor, Wilms' tumor, seminoma, ovarian tumor, leiomyomata tumor, cervical dysplasia, neuroblastoma, retinoblastoma, myelodysplastic syndrome, rhabdomyosarcoma, astrocytoma, malignant hypercalcemia, polycythemia vera, adenocarcinoma, glioblastoma multiforma, glioma, and malignant melanoma.

In some embodiments, the RIPK2-associated disease or disorder is a disease related to abnormal cell growth that is a non-malignant proliferative disease. In some embodiments, the non-malignant proliferative disease is benign prostatic hypertrophy, restenosis, hyperplasia, synovial proliferation disorder, idiopathic plasmacytic lymphadenopathy, or retinopathy.

In some embodiments, the RIPK2-associated disease or disorder is selected from the group consisting of: avascular necrosis, calcium pyrophosphate dihydrate crystal deposition disease (pseudo gout), Blau syndrome, Ehlers-Danlos syndrome, fibromyalgia, Fifth disease, giant cell arteritis, gout, Lyme disease, Marfan syndrome, myositis, osteoarthritis, osteogenesis imperfecta, osteoporosis, Paget's disease, Raynaud's phenomenon, reactive arthritis, reflex sympathetic dystrophy syndrome, spinal stenosis, and Still's disease.

In some embodiments, the RIPK2-associated disease or disorder is a cancer associated with chronic inflammation. In some embodiments, the cancer associated with chronic inflammation that can be treated (including reduction in the likelihood of recurrence) include colitis-associated colorectal cancer, gastric cancer, gastric mucosal lymphoma, lung cancer, hepatocellular carcinoma, thyroid cancer, breast cancer, oral cancer, head and neck cancer, nasopharyngeal carcinoma, endometrial cancer, uterine cancer, ovarian cancer, prostate cancer, bladder cancer, pancreatic cancer, esophageal cancer, skin cancer, and non-Hodgkin lymphoma.

Some embodiments provide a method of treating inflammatory bowel disease in a subject in need thereof, comprising administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating inflammatory bowel disease in a subject in need thereof, comprising (a) determining or having determined that the subject is suffering from inflammatory bowel disease; and (b) administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating inflammatory bowel disease in a subject in need thereof, comprising (a) determining or having determined that the subject has inflammatory bowel disease; and (b) administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating inflammatory bowel disease in a subject previously identified or diagnosed as having inflammatory bowel disease, the method comprising administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating Crohn's disease in a subject in need thereof, comprising administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating Crohn's disease in a subject in need thereof, comprising (a) determining or having determined that the subject is suffering from Crohn's disease; and (b) administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating Crohn's disease in a subject in need thereof, comprising (a) determining or having determined that the subject has Crohn's disease; and (b) administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

Some embodiments provide a method of treating Crohn's disease in a subject previously identified or diagnosed as having Crohn's disease, the method comprising administering to the subject an effective amount of a compound of Formula (I), or a pharmaceutically acceptable salt thereof, or a pharmaceutical composition comprising an effective amount of a compound of Formula (I), or a pharmaceutically acceptable salt thereof.

In some embodiments, the subject is a human.

### Inhibiting RIPK2 Activity and XIAP Binding

Some embodiments provide a method for inhibiting RIPK2 activity in a mammalian cell, comprising contacting the mammalian cell with a compound of Formula (I), or a pharmaceutically acceptable salt thereof. In some embodiments, the mammalian cell comprises a RIPK2 protein.

Also provided is a method for inhibiting RIPK2 activity in a mammalian cell comprising a RIPK2 protein, the method comprising contacting the mammalian cell with a compound of Formula (I), or a pharmaceutically acceptable salt thereof.

Also provided is a method for inhibiting the binding of a RIPK2 protein to a XIAP protein in a mammalian cell comprising a RIPK2 protein, the method comprising contacting the mammalian cell with a compound of Formula (I), or a pharmaceutically acceptable salt thereof.

In some embodiments, the contacting is in vitro. In some embodiments, the contacting is in vivo. In some embodiments, the amount of a compound of Formula (I), or a pharmaceutically acceptable salt thereof, is sufficient to inhibit RIPK2 activity in the cell. In some embodiments, the contacting is in vivo, wherein the method comprises administering a therapeutically effective amount of a compound of Formula (I), or a pharmaceutically acceptable salt thereof, to a subject having a mammalian cell having RIPK2 activity. In some embodiments, the mammalian cell is a mammalian immune cell. In some embodiments, the mammalian cell is an cancer cell.

In some embodiments, the RIPK2 activity is inhibited by about 10% to about 99%, for example, about 10% to about 50%, about 25% to about 75%, about 50% to about 99%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or any value in between.

As used herein, the term “contacting” refers to the bringing together of indicated moieties in an in vitro system or an in vivo system. For example, “contacting” RIPK2 (e.g., a RIPK2 protein) with a compound provided herein includes the administration of a compound provided herein to a subject, such as a human, having a RIPK2 protein, as well as, for example, introducing a compound provided herein into a sample containing a mammalian cellular or purified preparation containing a RIPK2 protein.

### Pharmaceutical Compositions

Some embodiments provide a pharmaceutical composition comprising a compound of Formula (I), or a pharmaceutically acceptable salt thereof, and at least one pharmaceutically acceptable excipient.

### NUMBERED EMBODIMENTS

Embodiment 1: A compound of Formula (I):

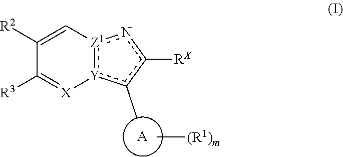

or a pharmaceutically acceptable salt thereof, wherein:
one or two of X, Y, and Z¹ is independently N and the other of X, Y, and Z¹ is C;
each [symbol] is a single bond or a double bond;
R^{X} is hydrogen, —NH₂, or halogen;
Ring A is phenyl or 5-10 membered heteroaryl;
m is 0, 1, 2, 3, or 4;
each R¹ is independently:
(i) C1-C6 alkoxy optionally substituted with hydroxyl or phenyl,
(ii) C1-C6 deuteroalkoxy,
(iii) C1-C6 alkoxyalkyl,
(iv) 5-6 membered heteroaryl,
(v) —N(R^{1A})—S(O₂)R^{1B},
(vi) —(C═O)NR^{1A}R^{1B},
(vii) halogen,
(viii) cyano,
(ix) hydroxyl,
(x) —NR^{A}R^{B},
(xi) C1-C6 alkyl optionally substituted with 1-2 substituents independently selected from hydroxyl and 4-8 membered heterocyclyl optionally substituted with hydroxyl or C1-C6 alkyl,
(xii) C1-C6 haloalkyl,
(xiii) C1-C6 haloalkoxy,
(xiv) C3-C6 cycloalkyl,
(xv) 4-8 membered heterocyclyl optionally substituted with 1-2 substituents independently selected from hydroxyl, C1-C6 alkyl, C1-C6 haloalkyl, and —(C═O)OC1-C6 alkyl,
(xvi) —S(O₂)C1-C6 alkyl, or
(xvii) 4-10 membered heterocyclyloxy optionally substituted with acyl,
(xviii) phenyl;
each R^{1A} is independently hydrogen or C1-C6 alkyl;
each R^{1B} is independently
(i) C1-C6 alkyl optionally substituted with C3-C6 cycloalkyl, 5-6 membered heteroaryl optionally substituted with C1-C6 alkyl, or 4-10 membered heterocyclyl optionally substituted with —(C═O)OC1-C6 alkyl,
(ii) 5-10 membered heteroaryl optionally substituted with 1-3 substituents independently selected from C1-C6 alkyl, hydroxyl, and C1-C6 hydroxyalkyl,
(iii) C3-C6 cycloalkyl optionally substituted with 1-2 substituents independently selected from C1-C6 alkyl and C1-C6 hydroxyalkyl,
(iv) ethylenyl,
(v) C1-C6 haloalkyl,
(vi) 4-10 membered heterocyclyl optionally substituted with —(C═O)OC1-C6 alkyl,
(vii) —NR^{1A}R^{1A}, or
(viii) phenyl;
R^{A} is hydrogen or C1-C6 alkyl;
R^{B} is
   (i) hydrogen,
   (ii) —S(O₂)C1-C6 alkyl,
   (iii) C3-C6 cycloalkyl optionally substituted with hydroxyl or C1-C6 alkoxy,
   (iv) —(C═O)C1-C6 alkyl,
   (v) —(C═O)OC1-C6 alkyl,
   (vi) 4-8 membered heterocyclyl optionally substituted with hydroxyl, or
   (vii) C1-C6 alkyl optionally substituted with 1-4 substituents independently selected from: halogen, hydroxyl, —NR^{C}R^{D}, C1-C6 alkoxy, C1-C6 haloalkoxy, C3-C6 cycloalkyl, phenyl optionally substituted with C1-C6 alkoxy, 5-6 membered heteroaryl optionally substituted with C1-C6 alkyl, and 4-8 membered heterocyclyl optionally substituted with —C(═O)C1-C6 alkyl or C1-C6 alkyl,
   (viii) —(C═O)5- or 6-membered heteroaryl optionally substituted with C1-C6 alkyl;
R² is
(i) hydrogen,
(ii) halogen,
(iii) C1-C6 alkoxy optionally substituted with 1-3 substituents independently selected from
   (a) hydroxyl,
   (b) halogen,
   (c) phosphate,
   (d) —NR^{2A}R^{2B},
   (e) 4-10 membered heterocyclyl optionally substituted with 1-3 substituents independently selected from hydroxyl, C1-C6 alkyl, C1-C6 haloalkyl, C1-C6 hydroxyalkyl, C1-C6 alkoxy, and C1-C6 alkoxyalkyl,
   (f) 5-6 membered heteroaryl optionally substituted with C1-C6 alkyl,
   (g) C3-C6 cycloalkyl optionally substituted with hydroxyl or hydroxyalkyl,
   (h) CO₂H,
   (i) C(O)R^{2A},
   (j) C1-C6 alkoxy; or
   (k) oxo;
(iv) C1-C6 haloalkoxy;
(v) 4-10 membered heterocyclyloxy,
(vi) —(C═O)NR^{2A}R^{2B},
(vii) C1-C6 alkyl optionally substituted with 1-3 substituents independently selected from hydroxyl, halogen, and —NR^{2A}R^{2B},
(viii) 5-6 membered heteroaryl optionally substituted with C1-C6 alkyl or C1-C6 alkoxy, or
(ix) —NR^{2A}R^{2B};
each R^{2A} and R^{2B} are independently hydrogen, C1-C6 alkyl optionally substituted with hydroxyl, or C1-C6 hydroxyalkyl;
R³ is
   (i) C1-C6 thioalkyl,

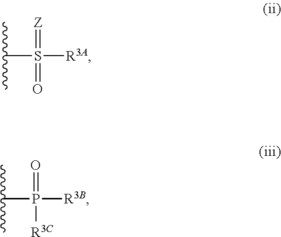

(iv) 4-8 membered heterocyclyl optionally substituted with 1-3 substituents independently selected from halogen, hydroxyl, C1-C6 alkyl, and C1-C6 alkoxy,
(v) C1-C6 alkyl optionally substituted with NR^{E}R^{F} or hydroxyl,
(vi) —CO₂H,
(vii) —C(═O)NR^{E}R^{F},
(viii) C1-C6 alkoxy optionally substituted with 4-10 membered heterocyclyl optionally substituted with C1-C6 alkoxy,
(ix) hydrogen,
(x) C1-C6 haloalkyl,
(xi) 5-6 membered heteroaryl optionally substituted with C1-C6 alkyl,
(xii) C1-C6 alkoxyalkyl, or
(xiii) C1-C6 hydroxyalkyl;
Z is O or NR⁴;
R^{3A} is
(i) C1-C6 haloalkyl,
(ii) C3-C6 cycloalkyl optionally substituted with C1-C6 alkyl,
(iii) C1-C6 alkyl optionally substituted with
   (a) C3-C6 cycloalkyl,
   (b) 5-6 membered heteroaryl optionally substituted with C1-C6 alkyl,
   (c) hydroxyl,
   (d) C1-C6 alkoxy, or
   (e) 4-6 membered heterocyclyl optionally substituted with 4-6 membered heterocyclyl or C1-C6 alkyl optionally substituted with C1-C6 alkoxy,
(iv) C1-C6 alkoxyalkyl,
(v) C1-C6 hydroxyalkyl,
(vi) 5-6 membered heteroaryl optionally substituted with C1-C6 alkyl, or
(vii) 4-10 membered heterocyclyl optionally substituted with 1-3 substituents independently selected from hydroxyl, C1-C6 alkyl, —C(O)OC1-C6 alkyl, and C1-C6 alkoxy,
(viii) —N(C1-C6 alkyl)₂;
R^{3B} and R^{3C} are each independently C3-C6 cycloalkyl or C1-C6 alkyl optionally substituted with C3-C6 cycloalkyl, or
R^{3B} and R^{3C} with the atom to which they are attached together form a 4-8 membered heterocyclyl optionally substituted with C1-C6 alkyl;
R⁴ is hydrogen or C1-C6 alkyl; and
each R^{C} and R^{D} are each independently hydrogen, —(C═O)C1-C6 alkyl, or C1-C6 alkyl optionally substituted with oxo;
each R^{E} and R^{F} are each independently hydrogen or C1-C6 alkyl, or R^{E} and R^{F} with the atom to which they are attached together form a 4-8 membered heterocyclyl optionally substituted with hydroxyl. Embodiment 2: The compound of Embodiment 1, wherein X is N.
Embodiment 3: The compound of Embodiment 1, wherein X is C.
Embodiment 4: The compound of any one of Embodiments 1-3, wherein Y is N.
Embodiment 5: The compound of any one of Embodiments 1-3, wherein Y is C.
Embodiment 6: The compound of any one of Embodiments 1-5, wherein Z¹ is N.
Embodiment 7: The compound of any one of Embodiments 1-5, wherein Z¹ is C.
Embodiment 8: The compound of any one of Embodiments 1-7, wherein Ring A is 5-11 membered heteroaryl.
Embodiment 9: The compound of any one of Embodiments 1-8, wherein Ring A is 5-6 membered heteroaryl.
Embodiment 10: The compound of any one of Embodiments 1-9, wherein Ring A is selected from the group consisting of pyrrolyl, pyrazolyl, imidazolyl, triazolyl, tetrazolyl, furanyl, thiophenyl, oxazolyl, isoxazolyl, isothiazolyl, thiazolyl, pyridinyl, pyrimidinyl, pyrazinyl, pyridazinyl, and pyridonyl.
Embodiment 11: The compound of any one of Embodiments 1-8, wherein Ring A is 9-10 membered heteroaryl.
Embodiment 12: The compound of any one of Embodiments 1-8 or 11, wherein Ring A is indole, indazole, aza-indole, benzimidazole, benzothiophene, benzoxazole, benzothiazole, benzopyrazole, pyrrolopyrimidine, benzoisoxazole, benzotriazole, purine, quinoline, isoquinoline, quinazoline, quinoxaline, or cinnoline.
Embodiment 13: The compound of any one of Embodiments 1-7, wherein Ring A is phenyl.
Embodiment 14: The compound of any one of Embodiments 1-13, wherein R^{X} is hydrogen.
Embodiment 15: The compound of any one of Embodiments 1-13, wherein R^{X} is halogen.
Embodiment 16: The compound of any one of Embodiments 1-13, wherein R^{X} is —NH₂.
Embodiment 17: The compound of any one of Embodiments 1-16, wherein one or more R¹ is C1-C6 alkoxy optionally substituted with hydroxyl or phenyl.
Embodiment 18: The compound of any one of Embodiments 1-16, wherein one or more R¹ is C1-C6 alkoxyalkyl.
Embodiment 19: The compound of any one of Embodiments 1-16, wherein one or more R¹ is 5-6 membered heteroaryl.
Embodiment 20: The compound of any one of Embodiments 1-16, wherein one or more R¹ is —N(R^{1A})—S(O₂)R^{1B}.
Embodiment 21: The compound of any one of Embodiments 1-16, wherein one or more R¹ is —(C═O)NR^{1A}R^{1B}.
Embodiment 22: The compound of any one of Embodiments 1-16, wherein one or more R¹ is halogen.
Embodiment 23: The compound of any one of Embodiments 1-16, wherein one or more R¹ is cyano.
Embodiment 24: The compound of any one of Embodiments 1-16, wherein one or more R¹ is hydroxyl.
Embodiment 25: The compound of any one of Embodiments 1-16, wherein one or more R¹ is —NR^{A}R^{B}.
Embodiment 26: The compound of any one of Embodiments 1-16, wherein one or more R¹ is C1-C6 alkyl optionally substituted with 1-2 substituents independently selected from hydroxyl and 4-8 membered heterocyclyl optionally substituted with hydroxyl or C1-C6 alkyl.
Embodiment 27: The compound of any one of Embodiments 1-16, wherein one or more R¹ is C1-C6 haloalkyl.
Embodiment 28: The compound of any one of Embodiments 1-16, wherein one or more R¹ is C1-C6 haloalkoxy.
Embodiment 29: The compound of any one of Embodiments 1-16, wherein one or more R¹ is C3-C6 cycloalkyl.
Embodiment 30: The compound of any one of Embodiments 1-16, wherein one or more R¹ is 4-8 membered heterocyclyl optionally substituted with 1-2 substituents independently selected from hydroxyl, C1-C6 a-kyl, C1-C6 haloalkyl, and —(C═O)OC1-C6 alkyl.
Embodiment 31: The compound of any one of Embodiments 1-16, wherein one or more R¹ is —S(O₂)C1-C6 alkyl.
Embodiment 32: The compound of any one of Embodiments 1-31, wherein one or more R^{1A} is hydrogen.
Embodiment 33: The compound of any one of Embodiments 1-31, wherein one or more R^{1A} is C1-C6 alkyl.
Embodiment 34: The compound of any one of Embodiments 1-33, wherein one or more R^{1B} is selected from C1-C6 alkyl optionally substituted with C3-C6 cycloalkyl, 5-6 membered heteroaryl optionally substituted with C1-C6 alkyl, or 4-10 membered heterocyclyl optionally substituted with —(C═O)OC1-C6 alkyl; 5-10 membered heteroaryl optionally substituted with 1-2 substituents independently selected from C1-C6 alkyl, hydroxyl, and C1-C6 hydroxyalkyl; C3-C6 cycloalkyl optionally substituted with 1-2 substituents independently selected from C1-C6 alkyl and C1-C6 hydroxyalkyl; ethylenyl; C1-C6 haloalkyl; 4-10 membered heterocyclyl optionally substituted with —(C═O)OC1-C6 alkyl; —NR^{1A}R^{1A}; and phenyl.
Embodiment 35: The compound of any one of Embodiments 1-33, wherein one or more R^{1B} is 5-10 membered heteroaryl optionally substituted with 1-2 substituents independently selected from C1-C6 alkyl, hydroxyl, and C1-C6 hydroxyalkyl.
Embodiment 36: The compound of any one of Embodiments 1-33, wherein one or more R^{1B} is C3-C6 cycloalkyl optionally substituted with 1-2 substituents independently selected from C1-C6 alkyl and C1-C6 hydroxyalkyl.
Embodiment 37: The compound of any one of Embodiments 1-33, wherein one or more R^{1B} is C3-C6 cycloalkyl optionally substituted with 1-2 substituents independently selected from C1-C6 alkyl and C1-C6 hydroxyalkyl.
Embodiment 38: The compound of any one of Embodiments 1-33, wherein one or more R^{1B} is ethylenyl.
Embodiment 39: The compound of any one of Embodiments 1-33, wherein one or more R^{1B} is C1-C6 haloalkyl.
Embodiment 40: The compound of any one of Embodiments 1-33, wherein one or more R^{1B} is 4-10 membered heterocyclyl optionally substituted with —(C═O)OC1-C6 alkyl.
Embodiment 41: The compound of any one of Embodiments 1-33, wherein one or more R^{1B} is —NR^{1A}R^{1A}.
Embodiment 42: The compound of any one of Embodiments 1-41, wherein R^{A} is hydrogen.
Embodiment 43: The compound of any one of Embodiments 1-41, wherein R^{A} is C1-C6 alkyl.
Embodiment 44: The compound of any one of Embodiments 1-43, wherein R^{B} is hydrogen.
Embodiment 45: The compound of any one of Embodiments 1-43, wherein R^{B} is —S(O₂)C1-C6 alkyl.
Embodiment 46: The compound of any one of Embodiments 1-43, wherein R^{B} is C3-C6 cycloalkyl optionally substituted with hydroxyl or C1-C6 alkoxy.
Embodiment 47: The compound of any one of Embodiments 1-43, wherein R^{B} is —(C═O)C1-C6 alkyl.
Embodiment 48: The compound of any one of Embodiments 1-43, wherein R^{B} is —(C═O)OC1-C6 alkyl.
Embodiment 49: The compound of any one of Embodiments 1-43, wherein R^{B} is 4-8 membered heterocyclyl optionally substituted with hydroxyl.
Embodiment 50: The compound of any one of Embodiments 1-43, wherein R^{B} is C1-C6 alkyl optionally substituted with 1-4 substituents independently selected from: halogen, hydroxyl, —NR^{C}R^{D}, C1-C6 alkoxy, C1-C6 haloalkoxy, C3-C6 cycloalkyl, phenyl optionally substituted with C1-C6 alkoxy, 5-6 membered heteroaryl optionally substituted with C1-C6 alkyl, and 4-8 membered heterocyclyl optionally substituted with —C(═O)C1-C6 alkyl or C1-C6 alkyl.
Embodiment 51: The compound of any one of Embodiments 1-43, wherein R^{B} is —(C═O)5- or 6-membered heteroaryl optionally substituted with C1-C6 alkyl.
Embodiment 52: The compound of any one of Embodiments 1-51, wherein R² is hydrogen.
Embodiment 53: The compound of any one of Embodiments 1-51, wherein R² is halogen.
Embodiment 54: The compound of any one of Embodiments 1-51, wherein R² is C1-C6 alkoxy optionally substituted with hydroxyl, phosphate, —NR^{2A}R^{2B}, 4-10 membered heterocyclyl optionally substituted with 1-3 substituents independently selected from hydroxyl, C1-C6 alkyl, C1-C6 hydroxyalkyl, and C1-C6 alkoxyalkyl, C3-C6 cycloalkyl optionally substituted with hydroxyl, CO₂H, or C(O)R^{2A}.
Embodiment 55: The compound of any one of Embodiments 1-51, wherein R² is C1-C6 haloalkoxy.
Embodiment 56: The compound of any one of Embodiments 1-51, wherein R² is 4-10 membered heterocyclyloxy.
Embodiment 57: The compound of any one of Embodiments 1-51, wherein R² is —(C═O)NR^{2A}R^{2B}.
Embodiment 58: The compound of any one of Embodiments 1-51, wherein R² is C1-C6 alkyl optionally substituted with 1-3 substituents independently selected from hydroxyl, halogen, and —NR^{2A}R^{2B}.
Embodiment 59: The compound of any one of Embodiments 1-51, wherein R² is 5-6 membered heteroaryl optionally substituted with C1-C6 alkyl or C1-C6 alkoxy.
Embodiment 60: The compound of any one of Embodiments 1-51, wherein R² is —NR^{2A}R^{2B}.
Embodiment 61: The compound of any one of Embodiments 1-60, wherein one or more R^{2A} is hydrogen.
Embodiment 62: The compound of any one of Embodiments 1-60, wherein one or more R^{2A} is C1-C6 alkyl.
Embodiment 63: The compound of any one of Embodiments 1-60, wherein one or more R^{2A} is C1-C6 hydroxyalkyl.
Embodiment 64: The compound of any one of Embodiments 1-63, wherein one or more R^{2B} is hydrogen.
Embodiment 65: The compound of any one of Embodiments 1-63, wherein one or more R^{2B} is C1-C6 alkyl.
Embodiment 66: The compound of any one of Embodiments 1-63, wherein one or more R^{2B} is C1-C6 hydroxyalkyl.
Embodiment 67: The compound of any one of Embodiments 1-51, wherein R² is selected from the group consisting of: H, OMe,

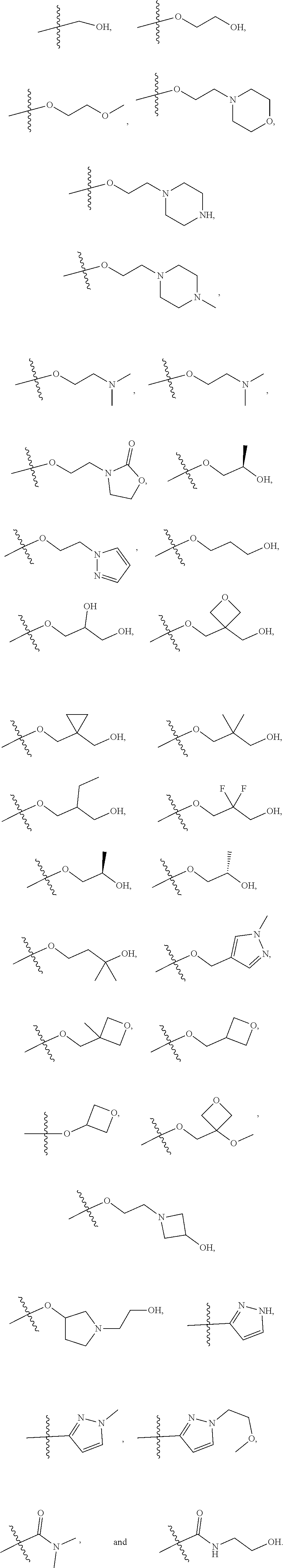

Embodiment 68: The compound of any one of Embodiments 1-67, wherein R³ is C1-C6 thioalkyl.
Embodiment 69: The compound of any one of Embodiments 1-67, wherein R³ is —CO₂H.
Embodiment 70: The compound of any one of Embodiments 1-67, wherein R³ is C1-C6 alkoxy optionally substituted with 4-10 membered heterocyclyl optionally substituted with C1-C6 alkoxy.
Embodiment 71: The compound of any one of Embodiments 1-67, wherein R³ is 4-8 membered heterocyclyl optionally substituted with 1-3 substituents independently selected from halogen, hydroxyl, C1-C6 alkyl, oxo, and C1-C6 alkoxy.
Embodiment 72: The compound of any one of Embodiments 1-67, wherein R³ is hydrogen.
Embodiment 73: The compound of any one of Embodiments 1-67, wherein R³ is C1-C6 haloalkyl.
Embodiment 74: The compound of any one of Embodiments 1-67, wherein R³ is 5-6 membered heteroaryl optionally substituted with C1-C6 alkyl.
Embodiment 75: The compound of any one of Embodiments 1-67, wherein R³ is C1-C6 alkoxyalkyl.
Embodiment 76: The compound of any one of Embodiments 1-67, wherein R³ is C1-C6 hydroxyalkyl.
Embodiment 77: The compound of any one of Embodiments 1-67, wherein R³ is C1-C6 alkyl optionally substituted with NR^{E}R^{F} or hydroxyl.
Embodiment 78: The compound of any one of Embodiments 1-67, wherein R³ is —C(═O)NR^{E}R^{F}.
Embodiment 79: The compound of any one of Embodiments 1-67, wherein R³ is

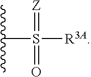

Embodiment 80: The compound of any one of Embodiments 1-67 or 79, wherein R^{3A} is selected from C1-C6 haloalkyl; C3-C6 cycloalkyl optionally substituted with C1-C6 alkyl; C1-C6 alkyl optionally substituted with C3-C6 cycloalkyl, 5-6 membered heteroaryl optionally substituted with C1-C6 alkyl, hydroxyl, C1-C6 alkoxy, or 4-6 membered heterocyclyl optionally substituted with 4-6 membered heterocyclyl or C1-C6 alkyl optionally substituted with C1-C6 alkoxy; C1-C6 alkoxyalkyl; C1-C6 hydroxyalkyl; 5-6 membered heteroaryl optionally substituted with C1-C6 alkyl; 4-10 membered heterocyclyl optionally substituted with 1-3 substituents independently selected from hydroxyl, C1-C6 alkyl, —C(O)OC1-C6 alkyl, and C1-C6 alkoxy; or —N(C1-C6 alkyl)₂.
Embodiment 81: The compound of any one of Embodiments 1-67 or 79, wherein R^{3A} is C3-C6 cycloalkyl optionally substituted with C1-C6 alkyl.
Embodiment 82: The compound of any one of Embodiments 1-67 or 79, wherein R^{3A} is C1-C6 alkyl optionally substituted with C3-C6 cycloalkyl, 5-6 membered heteroaryl optionally substituted with C1-C6 alkyl, hydroxyl, C1-C6 alkoxy, or 4-6 membered heterocyclyl optionally substituted with 4-6 membered heterocyclyl or C1-C6 alkyl optionally substituted with C1-C6 alkoxy.
Embodiment 83: The compound of any one of Embodiments 1-67 or 78, wherein R^{3A} is C1-C6 alkoxyalkyl
Embodiment 84: The compound of any one of Embodiments 1-67 or 79, wherein R^{3A} is C1-C6 hydroxyalkyl.
Embodiment 85: The compound of any one of Embodiments 1-67 or 79, wherein R^{3A} is 5-6 membered heteroaryl optionally substituted with C1-C6 alkyl.
Embodiment 86: The compound of any one of Embodiments 1-67 or 79, wherein R^{3A} is 4-10 membered heterocyclyl optionally substituted with 1-3 substituents independently selected from hydroxyl, C1-C6 alkyl, and C1-C6 alkoxy.
Embodiment 87: The compound of any one of Embodiments 1-41 or 47-49, wherein Z is NR⁴.
Embodiment 88: The compound of any one of Embodiments 1-41 or 47-51, wherein R⁴ is hydrogen.
Embodiment 89: The compound of any one of Embodiments 1-41 or 47-51, wherein R⁴ is C1-C6 alkyl.
Embodiment 90: The compound of any one of Embodiments 1-41 or 47-50, wherein Z is O.
Embodiment 91: The compound of any one of Embodiments 1-41, wherein R³

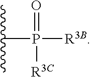

Embodiment 92: The compound of any one of Embodiments 1-41 or 91, wherein R^{3B} is C3-C6 cycloalkyl.
Embodiment 93: The compound of any one of Embodiments 1-41 or 91, wherein R^{3B} is or C1-C6 alkyl optionally substituted with C3-C6 cycloalkyl.
Embodiment 94: The compound of any one of Embodiments 1-41 or 91-93, wherein R^{3C} is C3-C6 cycloalkyl.
Embodiment 95: The compound of any one of Embodiments 1-41 or 91-93, wherein R^{3C} is or C1-C6 alkyl optionally substituted with C3-C6 cycloalkyl.
Embodiment 96: The compound of any one of Embodiments 1-95, wherein R^{C} is hydrogen.
Embodiment 97: The compound of any one of Embodiments 1-95, wherein R^{C} is C1-C6 alkyl optionally substituted with oxo.
Embodiment 98: The compound of any one of Embodiments 1-97, wherein R^{D} is hydrogen.
Embodiment 99: The compound of any one of Embodiments 1-97, wherein R^{D} is C1-C6 alkyl optionally substituted with oxo.
Embodiment 100: The compound of any one of Embodiments 1-99, wherein R^{E} is hydrogen.
Embodiment 101: The compound of any one of Embodiments 1-99, wherein R^{E} is C1-C6 alkyl.
Embodiment 102: The compound of any one of Embodiments 1-101, wherein R^{F} is hydrogen.
Embodiment 103: The compound of any one of Embodiments 1-101, wherein R^{F} is C1-C6 alkyl.
Embodiment 104: The compound of any one of Embodiments 1-99, wherein R^{E} and R^{F} with the atom to which they are attached together form a 4-8 membered heterocyclyl optionally substituted with hydroxyl.
Embodiment 105: The compound of any one of Embodiments 1-67, wherein R³ is selected from the group consisting of: H, OMe, CF₃,

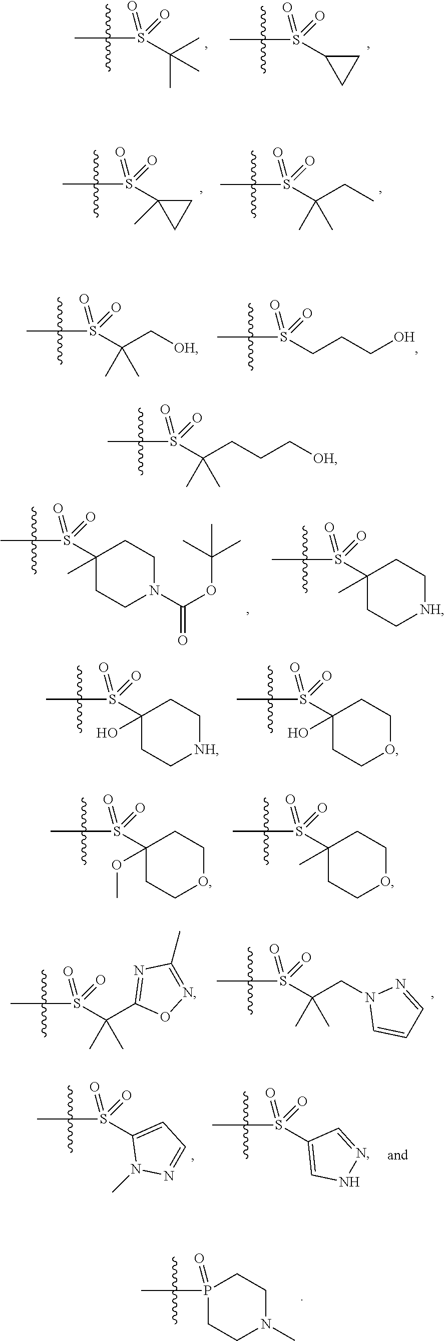

Embodiment 106: The compound of Embodiment 1, wherein Formula (I) is (I-a):

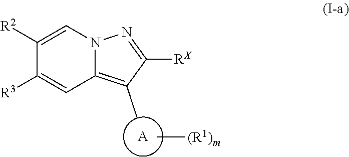

or a pharmaceutically acceptable salt thereof.
Embodiment 107: The compound of Embodiment 1, wherein Formula (I) is (I-b):

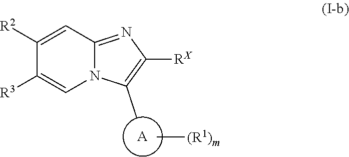

or a pharmaceutically acceptable salt thereof.
Embodiment 108: The compound of Embodiment 1, wherein Formula (I) is (I-c):

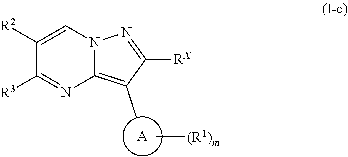

or a pharmaceutically acceptable salt thereof.
Embodiment 109: The compound of Embodiment 1, wherein Formula (I) is (I-d):

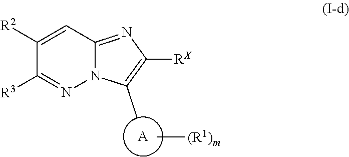

or a pharmaceutically acceptable salt thereof.
Embodiment 110: The compound of Embodiment 1, wherein Formula (I) is (I-e):

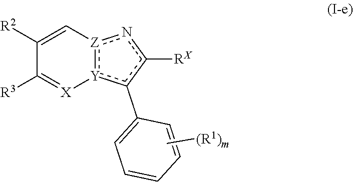

or a pharmaceutically acceptable salt thereof.
Embodiment 111: The compound of Embodiment 1, wherein Formula (I) is (I-f):

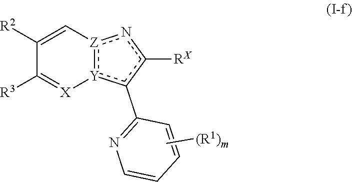

or a pharmaceutically acceptable salt thereof.
Embodiment 112: The compound of Embodiment 1, wherein Formula (I) is (I-g):

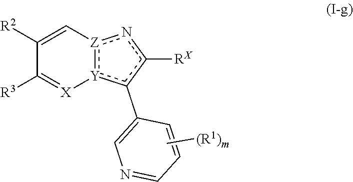

or a pharmaceutically acceptable salt thereof.
Embodiment 113: The compound of Embodiment 1, wherein Formula (I) is (I-h):

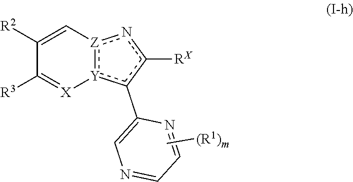

or a pharmaceutically acceptable salt thereof.
Embodiment 114: The compound of Embodiment 1, wherein Formula (I) is (I-i):

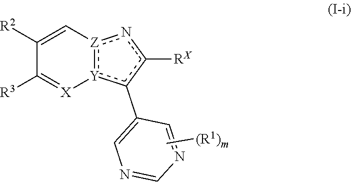

or a pharmaceutically acceptable salt thereof.
Embodiment 115: The compound of Embodiment 1, wherein Formula (I) is (I-j):

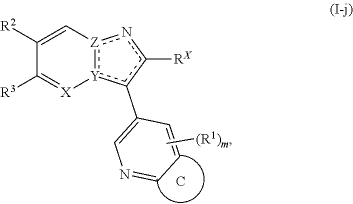

or a pharmaceutically acceptable salt thereof, wherein ring C is morpholine or N-methylmorpholine.
Embodiment 116: The compound of Embodiment 1, wherein Formula (I) is (I-k):

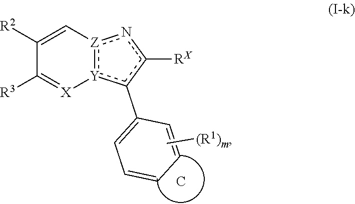

or a pharmaceutically acceptable salt thereof, wherein ring C is pyridine or pyrrolidinone.
Embodiment 117: The compound of Embodiment 1, wherein

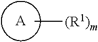

is selected from the group consisting of:

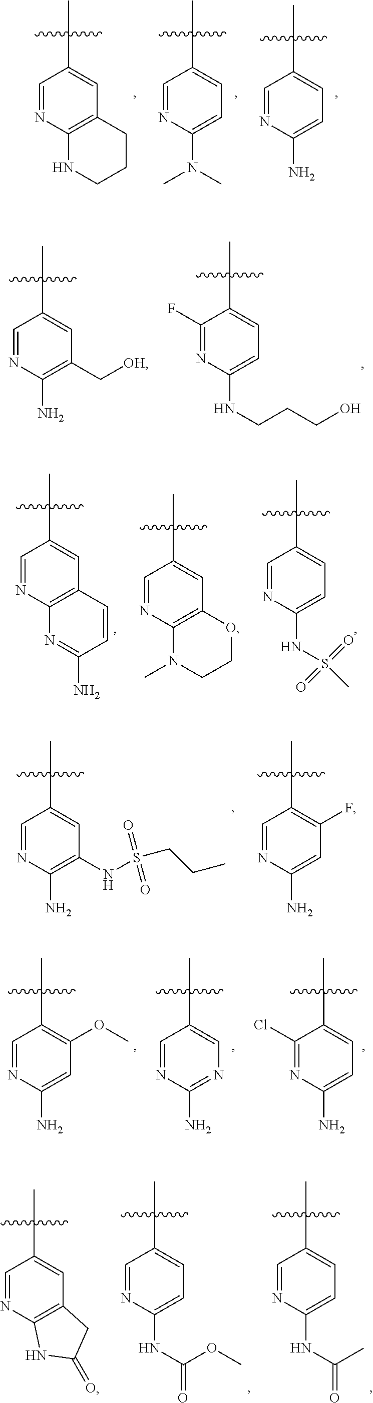

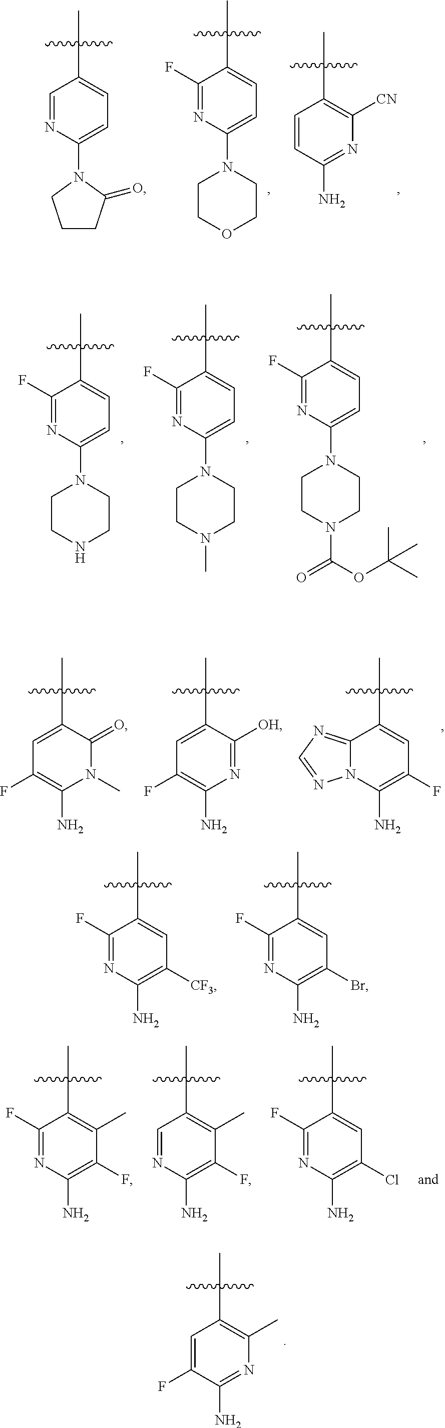

Embodiment 118: The compound of Embodiment 1, wherein

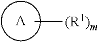

is selected from the group consisting of:

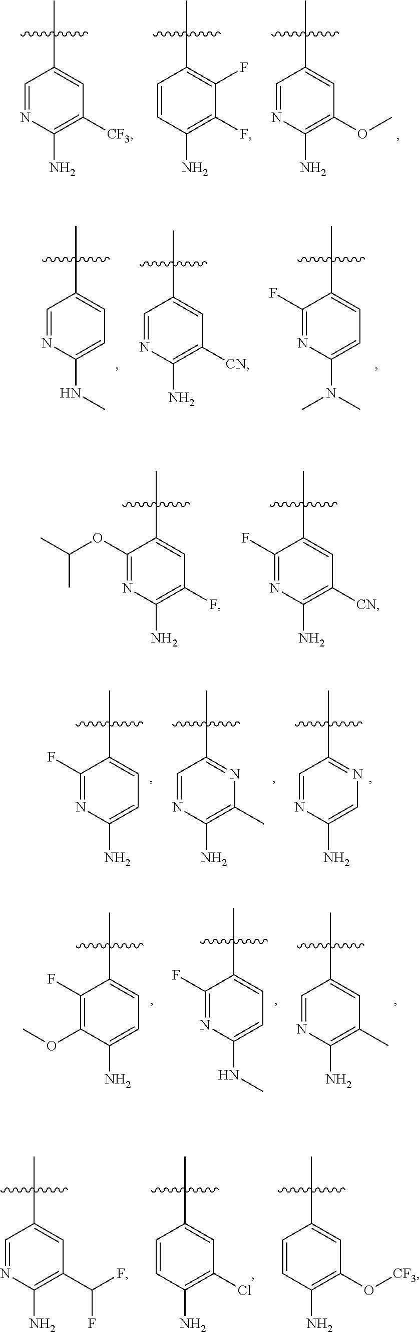

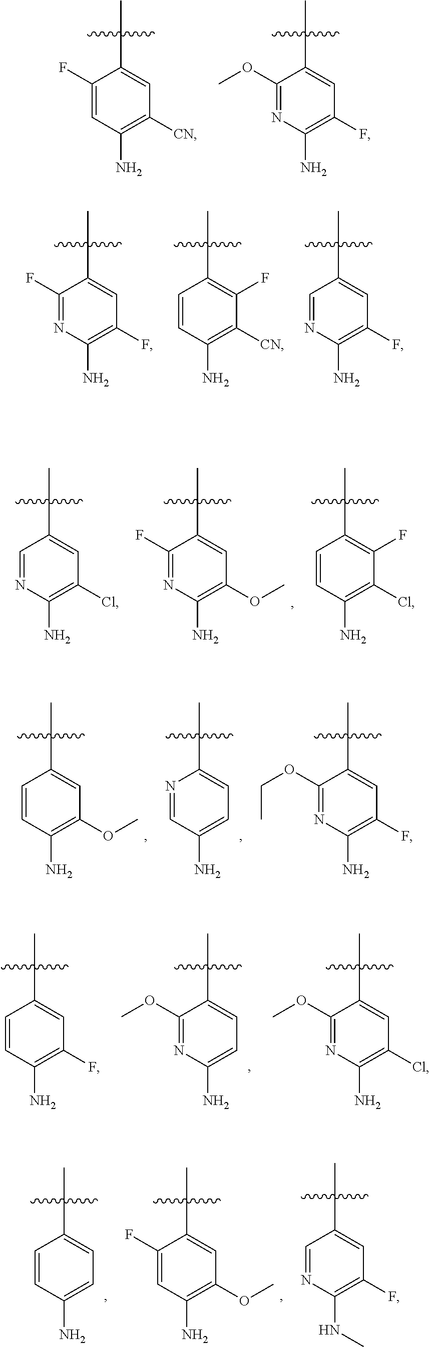

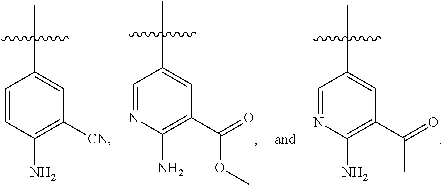

Embodiment 119: The compound of Embodiment 1, wherein

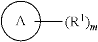

is selected from the group consisting of:

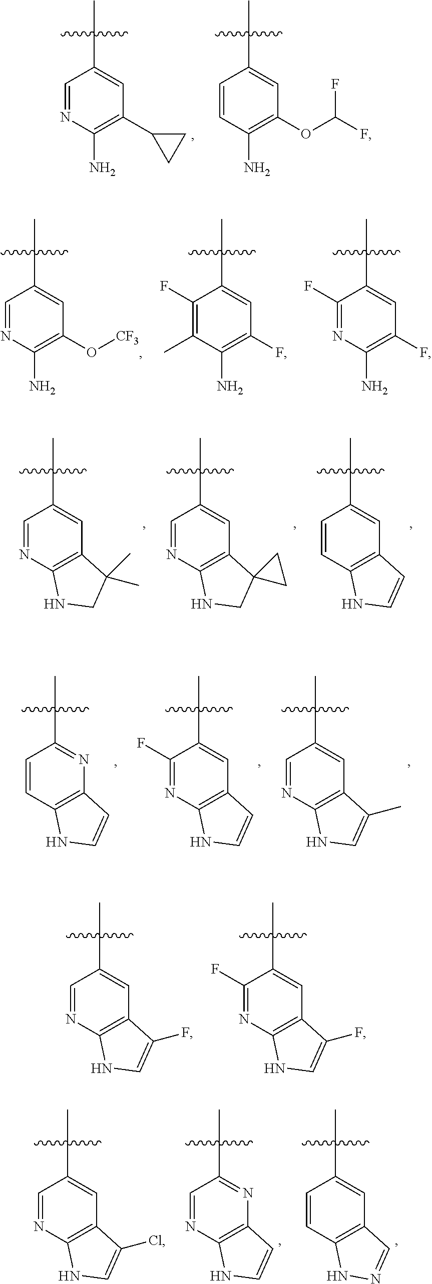

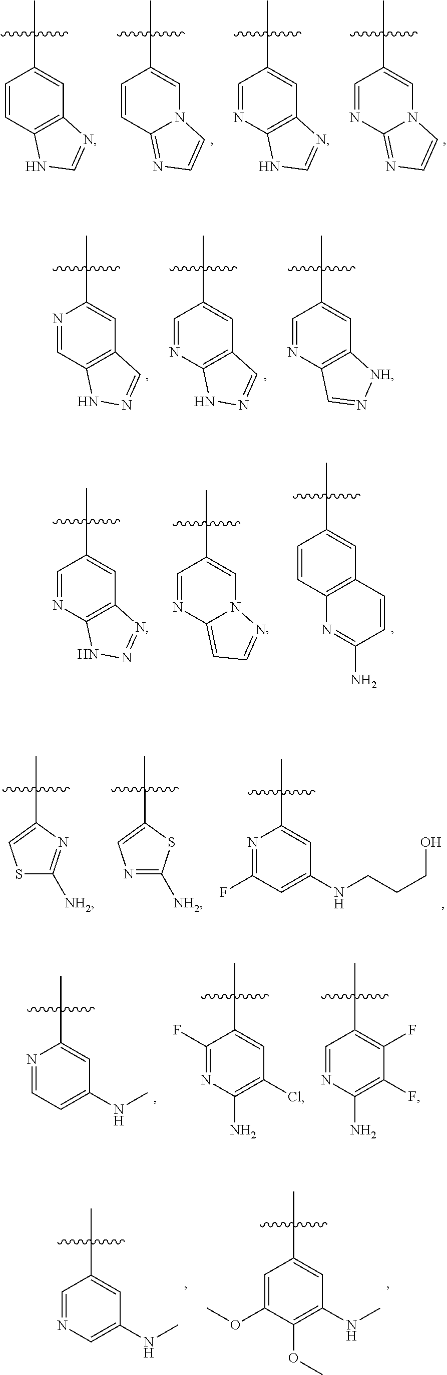

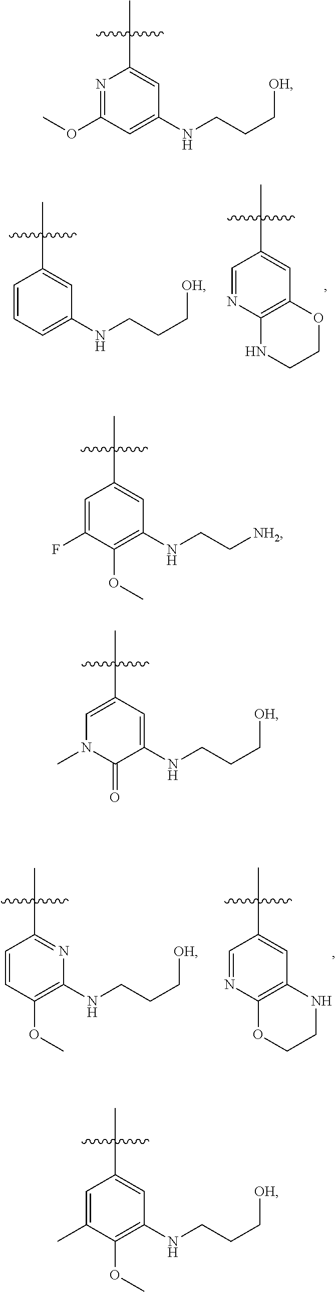

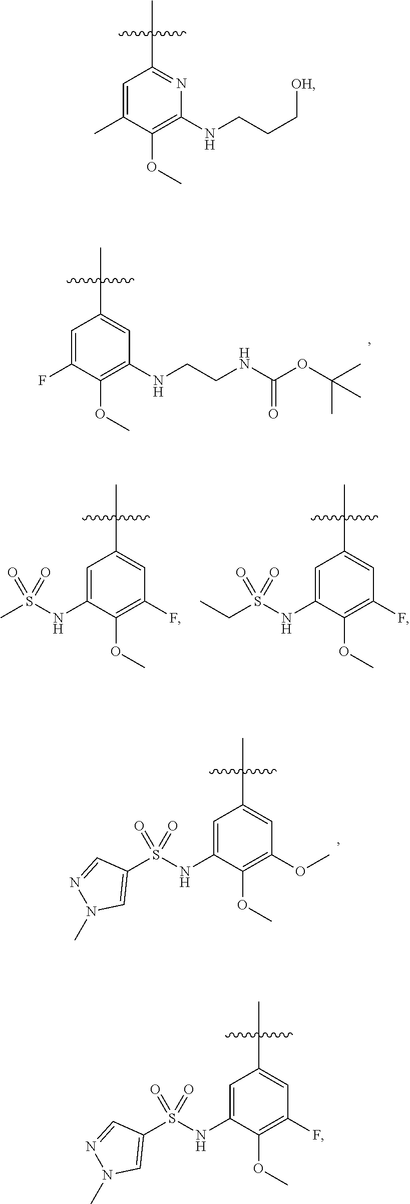

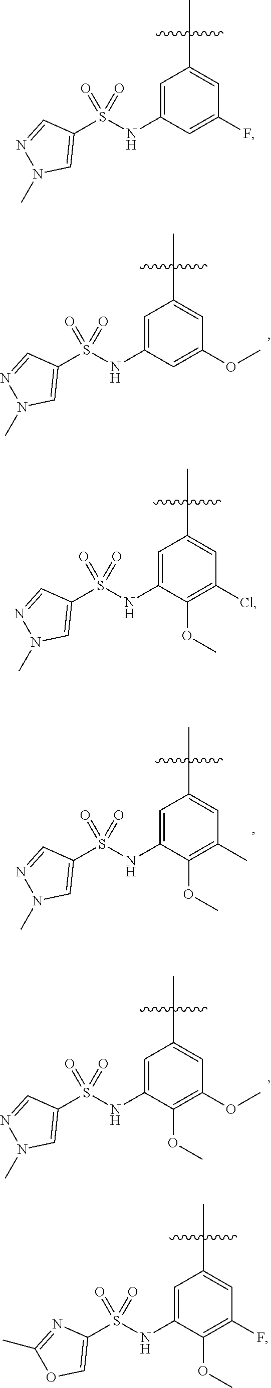

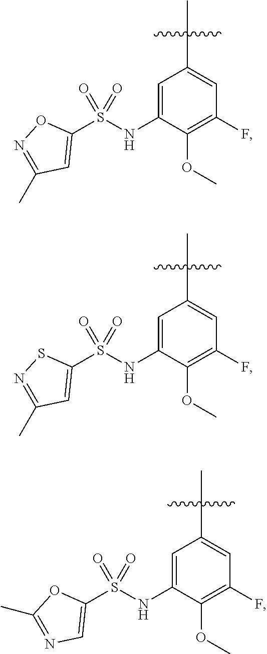 [SMILES: *c1cc(F)c(OC)c(NS(=O)(=O)c2cc(C)no2)c1 ; *c1cc(F)c(OC)c(NS(=O)(=O)c2cc(C)ns2)c1 ; *c1cc(F)c(OC)c(NS(=O)(=O)c2cnc(C)o2)c1]

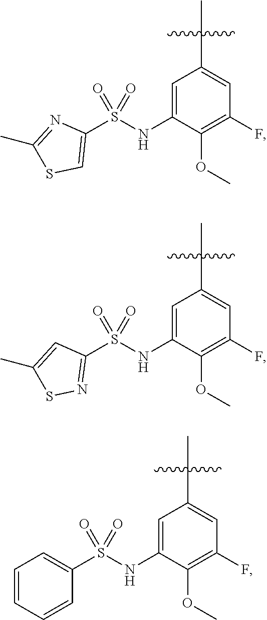 [SMILES: *c1cc(F)c(OC)c(NS(=O)(=O)c2csc(C)n2)c1 ; *c1cc(F)c(OC)c(NS(=O)(=O)c2cc(C)sn2)c1 ; *c1cc(F)c(OC)c(NS(=O)(=O)c2ccccc2)c1]

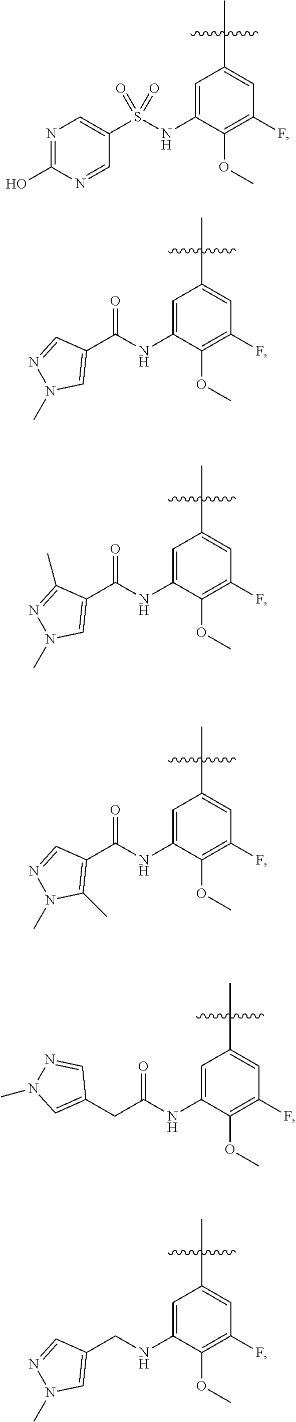 [SMILES: *c1cc(F)c(OC)c(NS(=O)(=O)c2cnc(O)nc2)c1 ; *c1cc(F)c(OC)c(NC(=O)c2cnn(C)c2)c1 ; *c1cc(F)c(OC)c(NC(=O)c2cn(C)nc2C)c1 ; *c1cc(F)c(OC)c(NC(=O)c2cnn(C)c2C)c1 ; *c1cc(F)c(OC)c(NC(=O)Cc2cnn(C)c2)c1 ; *c1cc(F)c(OC)c(NCc2cnn(C)c2)c1]

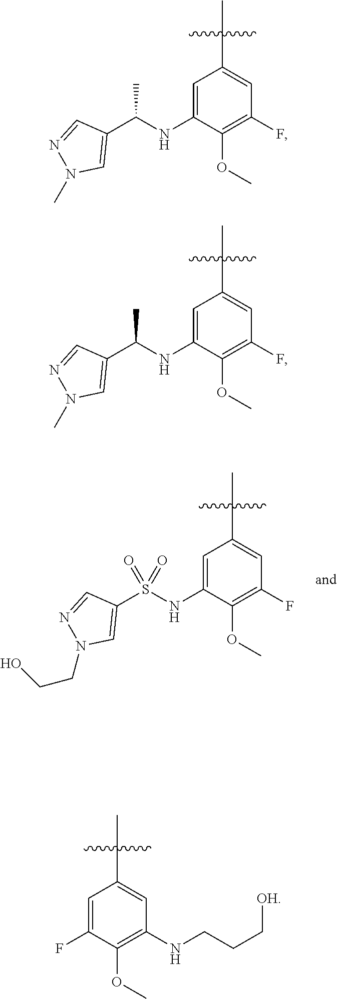 [SMILES: *c1cc(F)c(OC)c(N[C@@H](C)c2cnn(C)c2)c1 ; *c1cc(F)c(OC)c(N[C@H](C)c2cnn(C)c2)c1 ; *c1cc(F)c(OC)c(NS(=O)(=O)c2cnn(CCO)c2)c1 ; *c1cc(F)c(OC)c(NCCCO)c1]

Embodiment 120: The compound of Embodiment 1, wherein

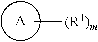

is selected from the group consisting of:

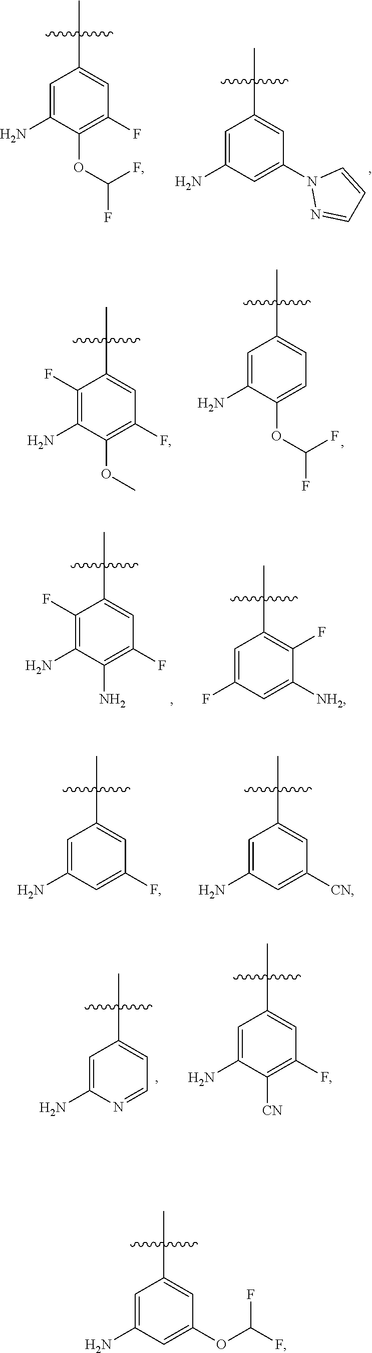

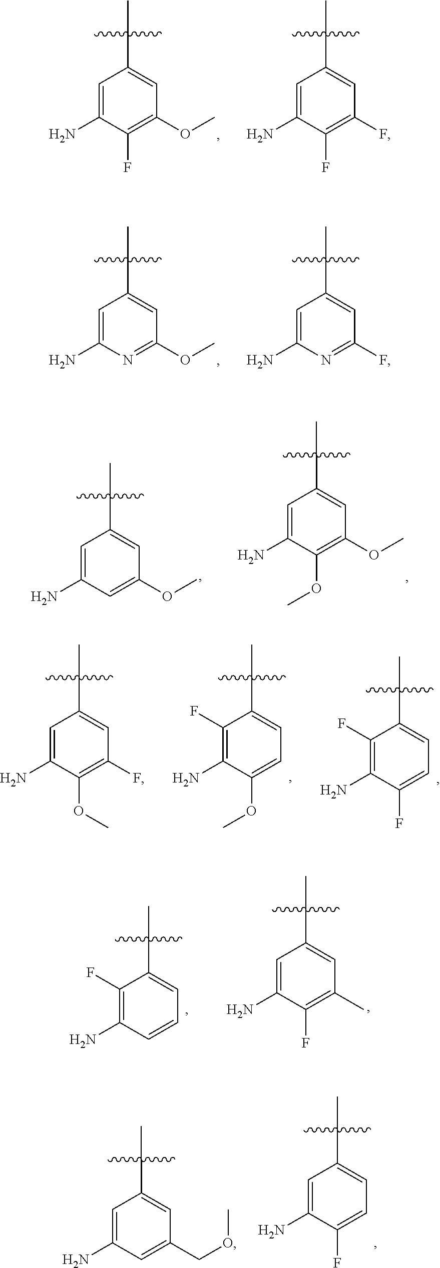

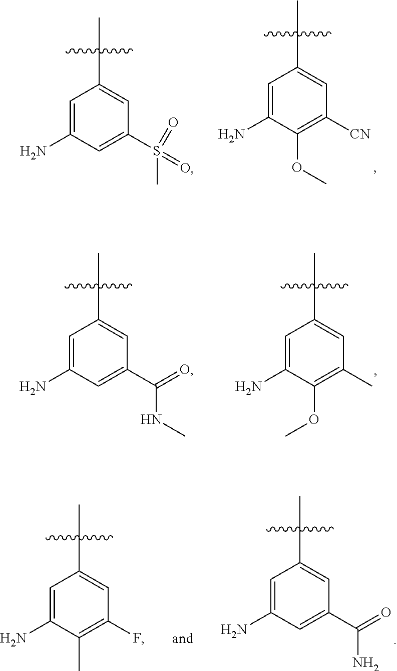

Embodiment 121: The compound of Embodiment 1, wherein

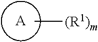

is selected from the group consisting of:

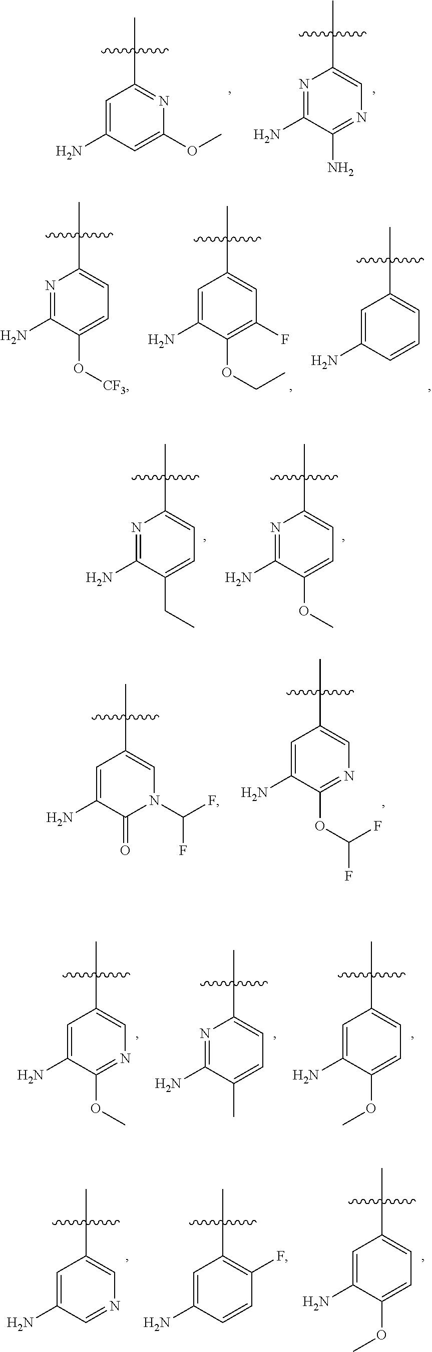

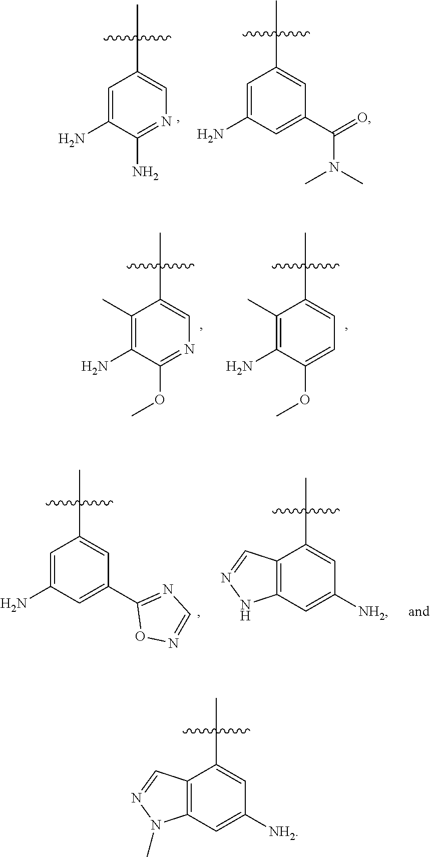

Embodiment 122: A compound selected from the group consisting of the compounds in Examples 1-304, or a pharmaceutically acceptable salt thereof.
Embodiment 123: A pharmaceutical composition comprising a compound of any one of Embodiments 1-122, or a pharmaceutically acceptable salt thereof, and one or more pharmaceutically acceptable excipients.
Embodiment 124: A method of treating a RIPK2-associated disease or disorder in a subject in need thereof, comprising administering to the subject an effective amount of a compound of any one of Embodiments 1-122, or a pharmaceutically acceptable salt thereof, or the pharmaceutical composition of Embodiment 123.
Embodiment 125: The method of Embodiment 124, wherein the RIPK2-associated disease or disorder is a cardiovascular disease, an allergic disorder, an autoimmune disease, an inflammatory disease, a cardiovascular disease, a fibrotic disease, or a disease associated with abnormal cell growth.
Embodiment 126: The method of Embodiment 124 or 125, wherein the RIPK2-associated disease or disorder is an inflammatory disease.
Embodiment 127: The method of Embodiment 126, wherein the inflammatory disease is chronic lung inflammatory disease, osteoarthritis, inflammatory arthritis, asthma, early onset sarcoidosis, sarcoidosis, eczema, allergic eczema, uveitis, reactive arthritis, chronic inflammation, chronic prostatitis, inflammatory bowel disease, glomerulonephritis, bursitis, carpal tunnel syndrome, tendinitis, inflammation of the lung (e.g., chronic obstructive pulmonary disease), pelvic inflammatory disease, transplant rejection, vasculitis, regional enteritis, distal ileitis, regional ileitis, and terminal ileitis, central areolar choroidal dystrophy, macular degeneration, retinosis pigmentosa, adult vitelliform disease, pattern dystrophy, diabetic retinopathy, BEST disease, myopic degeneration, central serous retinopathy, Stargardt's disease, Cone-Rod dystrophy, North Carolina dystrophy, infectious retinitis, inflammatory retinitis, uveitis, toxic retinitis, or systemic inflammatory response syndrome.
Embodiment 128: A method of treating inflammatory bowel disease in a subject in need thereof, comprising administering to the subject an effective amount of the compound of any one of Embodiments 1-122, or a pharmaceutically acceptable salt thereof, or the pharmaceutical composition of Embodiment 123.
Embodiment 129: A method of treating inflammatory bowel disease in a subject in need thereof, comprising (a) determining that the subject is suffering from inflammatory bowel disease; and (b) administering to the subject an effective amount of the compound of the compound of any one of Embodiments 1-122, or a pharmaceutically acceptable salt thereof, or the pharmaceutical composition of Embodiment 123.
Embodiment 130: A method of treating inflammatory bowel disease in a subject previously identified or diagnosed as having inflammatory bowel disease, the method comprising administering to the subject an effective amount of the compound of the compound of any one of Embodiments 1-122, or a pharmaceutically acceptable salt thereof, or the pharmaceutical composition of Embodiment 123.
Embodiment 131: A method of treating Crohn's disease in a subject in need thereof, comprising administering to the subject an effective amount of the compound of the compound of any one of Embodiments 1-122, or a pharmaceutically acceptable salt thereof, or the pharmaceutical composition of Embodiment 123.
Embodiment 132: A method of treating Crohn's disease in a subject in need thereof, comprising (a) determining that the subject is suffering from Crohn's disease; and (b) administering to the subject an effective amount of the compound of the compound of any one of Embodiments 1-122, or a pharmaceutically acceptable salt thereof, or the pharmaceutical composition of Embodiment 123.
Embodiment 133: A method of treating Crohn's disease in a subject previously identified or diagnosed as having Crohn's disease, the method comprising administering to the subject an effective amount of the compound of the compound of any one of Embodiments 1-122, or a pharmaceutically acceptable salt thereof, or the pharmaceutical composition of Embodiment 123.

### EXAMPLES

### Materials and Methods

The compounds provided herein, including salts thereof, can be prepared using known organic synthesis techniques and can be synthesized according to any of numerous possible synthetic routes.

The reactions for preparing the compounds provided herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of the compounds provided herein can involve the protection and deprotection of various chemical groups. The need for protection and deprotection, and the selection of appropriate protecting groups, can be readily determined by one skilled in the art. The chemistry of protecting groups can be found, for example, in Protecting Group Chemistry, 1^{st} Ed., Oxford University Press, 2000; March's Advanced Organic Chemistry: Reactions, Mechanisms, and Structure, 5^{th} Ed., Wiley-Interscience Publication, 2001; and Peturssion, S. et al., “Protecting Groups in Carbohydrate Chemistry,” J. Chem. Educ., 74(11), 1297 (1997).

### Intermediates

### Intermediate 1

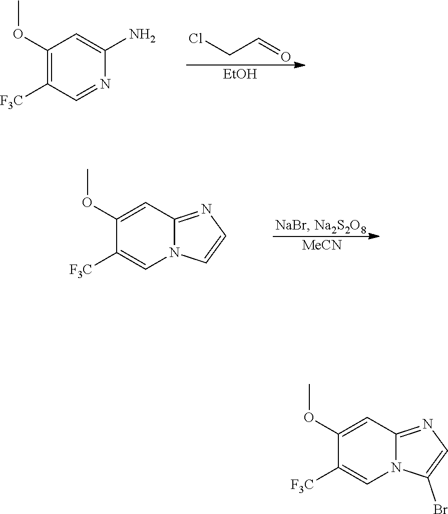

7-methoxy-6-(trifluoromethyl)imidazo[1,2-a]pyridine: To a 30 mL vial was added 4-methoxy-5-(trifluoromethyl)pyridin-2-amine (0.2 g, 1.04 mmol, 1 eq) and chloroacetaldehyde (0.095 mL, 1.35 mmol) in EtOH (6 mL) at rt under a N₂ atmosphere. The vial was sealed and then heated at 80° C. for 18 h before it was cooled to rt and concentrated under reduced pressure. The mixture was then diluted with water (10 mL) and extracted with EtOAc (2×15 mL). The organic layer was washed with brine, dried over Mg₂SO₄, filtered, and concentrated under reduced pressure. The crude compound was purified by column chromatography (SiO₂, 5-15% EtOAc/petroleum ether) to provide the title compound (180 mg, 80%) as a light-yellow solid. [M+H]⁺=217.1. ¹H NMR (400 MHz, DMSO-d₆) δ 9.42 (s, 1H), 8.13 (s, 1H), 8.03 (d, J=1.6 Hz, 1H), 7.44-7.44 (m, 1H), 4.09 (s, 3H).

3-bromo-7-methoxy-6-(trifluoromethyl)imidazo[1,2-a]pyridine: A suspension of 7-methoxy-6-(trifluoromethyl)imidazo[1,2-a]pyridine (120 mg, 0.56 mmol, 1 eq), sodium bromide, (114 mg, 1.11 mmol, 2 eq) and sodium persulfate (132 mg, 0.56 mmol) in MeCN (2.4 mL) was heated at 80° C. for 5 h. The reaction was cooled to rt, diluted with EtOAc (20 mL) and washed with water (3×5 mL). The organic layer was separated, dried over Mg₂SO₄, filtered, and concentrated under reduced pressure. The crude compound was purified by column chromatography (SiO₂, 20-30% EtOAc/petroleum ether) to provide the title compound (90 mg, 55%) as an off-white solid. [M+H]⁺=295.2. ¹H NMR (400 MHz, DMSO-d₆) δ 8.55 (s, 1H), 7.87 (s, 1H), 7.37 (s, 1H), 4.00 (s, 3H).

### Intermediate 2

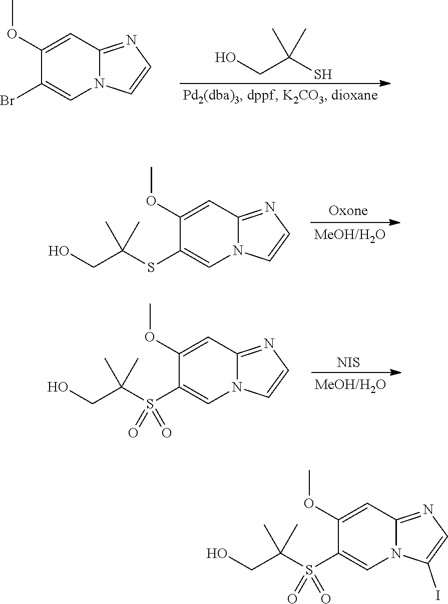

2-((7-methoxyimidazo[1,2-a]pyridin-6-yl)thio)-2-methylpropan-1-ol: A mixture of 6-bromo-7-methoxyimidazo[1,2-a]pyridine (50 mg, 198.19 μmol, 1 eq), K₂CO₃ (82.17 mg, 594.56 μmol, 3 eq), dppf (10.99 mg, 19.82 μmol, 0.1 eq) and Pd₂(dba)₃ (9.07 mg, 9.91 μmol, 0.05 eq) in dioxane (1 mL) was degassed and purged with N₂ (3×). 2-mercapto-2-methylpropan-1-ol (23.15 mg, 218.01 μmol, 1.1 eq) was added, and then the mixture was stirred at 100° C. for 12 hours under a N₂ atmosphere. The reaction mixture was diluted with H₂O (10 mL) and extracted with EtOAc (3×10 mL). The combined organic layers were washed with brine (15 mL), dried over Na₂SO₄, filtered and concentrated in vacuo. The resulting crude material was purified by column chromatography (SiO₂, 0-10% MeOH/DCM) to yield the title compound as a brown solid. LCMS [M+H]⁺=253.0. ¹H NMR (400 MHz, DMSO-d₆) δ ppm 8.70 (s, 1H), 7.76 (s, 1H), 7.41 (s, 1H), 6.96 (s, 1H), 4.78 (t, J=6.0 Hz, 1H), 3.85 (s, 3H), 3.27 (d, J=6.0 Hz, 2H), 1.13 (s, 6H).

2-((7-methoxyimidazo[1,2-a]pyridin-6-yl)sulfonyl)-2-methylpropan-1-ol: To a solution of 2-((7-methoxyimidazo[1,2-a]pyridin-6-yl)thio)-2-methylpropan-1-ol (280 mg, 998.68 μmol, 1 eq) in MeOH (7 mL) and H₂O (7 mL) was added Oxone® (1.84 g, 3.00 mmol, 3 eq) at 0° C. The mixture was stirred at 25° C. for 12 h to yield the title compound as a brown liquid, which was used in the next step without further purification.

2-((3-iodo-7-methoxyimidazo[1,2-a]pyridin-6-yl)sulfonyl)-2-methylpropan-1-ol: To a solution of 2-((7-methoxyimidazo[1,2-a]pyridin-6-yl)sulfonyl)-2-methylpropan-1-ol (280 mg, 886.29 μmol, 1.0 eq) in MeOH (7 mL) and H₂O (14 mL) was added NIS (299.10 mg, 1.33 mmol, 1.5 eq). The mixture was stirred at 25° C. for 2 h before it was quenched with saturated NaHSO₃ (20 mL) at 25° C. The resulting mixture was diluted with H₂O (25 mL) and extracted with DCM (3×30 mL). The combined organic layers were dried over Na₂SO₄, filtered and concentrated in vacuo. The resulting crude material was purified by column chromatography (SiO₂, petroleum ether/EtOAc=100/1 to 0/1) to yield the title compound as a brown solid. LCMS [M+H]⁺=410.9. ¹H NMR (400 MHz, DMSO-d₆) δ ppm 8.50 (s, 1H), 7.71 (s, 1H), 7.23 (s, 1H), 4.99 (t, J=5.6 Hz, 1H), 3.92 (s, 3H), 3.57 (d, J=5.6 Hz, 2H), 1.29 (s, 6H).

### Intermediate 3

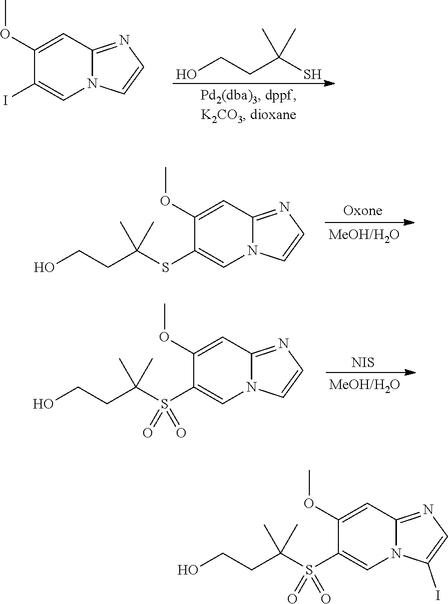

3-((7-methoxyimidazo[1,2-a]pyridin-6-yl)thio)-3-methylbutan-1-ol: To a solution of 6-iodo-7-methoxyimidazo[1,2-a]pyridine (0.5 g, 1.98 mmol, 1 eq), K₂CO₃ (821.74 mg, 5.95 mmol, 3 eq), DPPF (109.87 mg, 198.19 μmol, 0.1 eq), Pd₂(dba)₃ (90.74 mg, 99.09 μmol, 0.05 eq) in 1,4-dioxane (10 mL) was added 3-mercapto-3-methylbutan-1-ol (262.07 mg, 2.18 mmol, 1.1 eq) under a N₂ atmosphere. The mixture was stirred at 100° C. for 12 hours before it was diluted with H₂O (10 mL) and extracted with EtOAc (3×10 mL). The combined organic layers were washed with brine (15 mL), dried over Na₂SO₄, filtered and concentrated under reduced pressure to give a residue, which was purified by column chromatography (SiO₂, 10% MeOH/DCM) to give the title compound (283 mg, 48% yield) as a brown oil. ¹H NMR (400 MHz, DMSO-d₆) δ ppm 8.66 (s, 1H), 7.77 (s, 1H), 7.42 (s, 1H), 6.96 (s, 1H), 4.38 (t, J=7.2 Hz, 1H), 3.84 (s, 3H), 3.63-3.56 (m, 2H), 1.65 (t, J=7.2 Hz, 2H), 1.21-1.16 (m, 6H).

3-((7-methoxyimidazo[1,2-a]pyridin-6-yl)sulfonyl)-3-methylbutan-1-ol: A mixture of 3-((7-methoxyimidazo[1,2-a]pyridin-6-yl)thio)-3-methylbutan-1-ol (283 mg, 956.23 μmol, 1 eq), Oxone® (1.76 g, 2.87 mmol, 3 eq) in MeOH (6 mL) and H₂O (2 mL) was degassed and purged with N₂ 3 times, and then the mixture was stirred at 25° C. for 12 hours under a N₂ atmosphere. The reaction mixture for the title compound (280 mg) was used for the next step directly. [M+H]⁺=298.9.

3-((3-iodo-7-methoxyimidazo[1,2-a]pyridin-6-yl)sulfonyl)-3-methylbutan-1-ol: A mixture of 3-((7-methoxyimidazo[1,2-a]pyridin-6-yl)sulfonyl)-3-methylbutan-1-ol (280 mg, 938.47 μmol, 1 eq), NIS (316.71 mg, 1.41 mmol, 1.5 eq) in MeOH (6 mL) and H₂O (4 mL) was degassed and purged with N₂ 3 times, and then the mixture was stirred at 25° C. for 1 hours under a N₂ atmosphere. The reaction mixture was quenched with H₂O (10 mL), and then diluted with more water (20 mL) and extracted with EtOAc (3×20 mL). The water phase was lyophilized to give a residue, which was purified by column chromatography (SiO₂, 10% MeOH/DCM) to give the title compound (350 mg, 79% yield) as a yellow solid. ¹H NMR (400 MHz, DMSO-d₆) δ ppm 8.59-8.42 (m, 1H), 8.01-7.86 (m, 1H), 7.37 (s, 1H), 3.99 (s, 3H), 3.56 (t, J=6.8 Hz, 2H), 1.84 (t, J=6.8 Hz, 2H), 1.33 (s, 6H).

### Intermediate 4

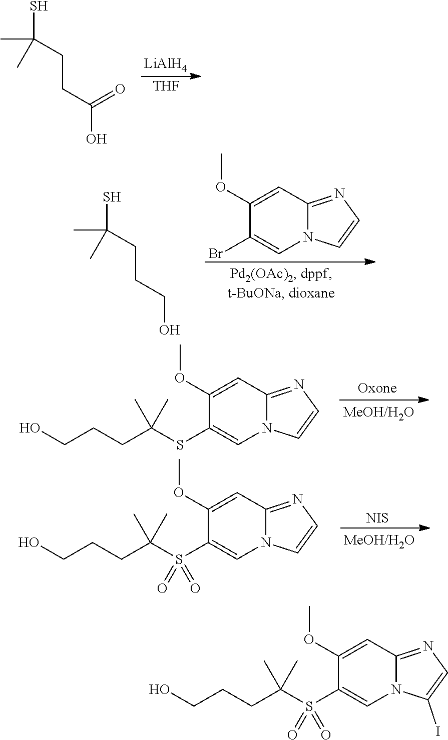

4-mercapto-4-methylpentan-1-ol: To a solution of 4-mercapto-4-methylpentanoic acid (500 mg, 3.37 mmol, 1 eq) in THF (2 mL) was added LiAlH₄ (2.5 M, 2.70 mL, 2 eq) at 0° C. The mixture was stirred at 20° C. for 1 hour before it was quenched with EtOAc (2 mL), and then then diluted with H₂O (0.5 mL) and extracted with EtOAc (3×5 mL). The combined organic layers were dried over Na₂SO₄, filtered and concentrated under reduced pressure to give a residue, which was purified by column chromatography (SiO2, petroleum ether/EtOAc=20/1 to 3/1) to give the title compound (147 mg, 29% yield) as a colorless oil. ¹H NMR (400 MHz, DMSO-d₆) δ ppm 4.41 (t, J=5.6 Hz, 1H), 3.41-3.35 (m, 2H), 1.55-1.50 (m, 4H), 1.31 (s, 6H).

4-((7-methoxyimidazo[1,2-a]pyridin-6-yl)thio)-4-methylpentan-1-ol: A mixture of 4-mercapto-4-methylpentan-1-ol (143.07 mg, 959.23 μmol, 1.1 eq), 6-bromo-7-methoxyimidazo[1,2-a]pyridine (220 mg, 872.03 μmol, 1 eq), Pd(OAc)₂ (19.58 mg, 87.20 μmol, 0.1 eq), dppf (96.69 mg, 174.41 μmol, 0.2 eq) and t-BuONa (251.41 mg, 2.62 mmol, 3 eq) in dioxane (7 mL) was degassed and purged with N₂ 3 times. The mixture was stirred at 90° C. for 12 hours under a N₂ atmosphere before it was filtered and concentrated under reduced pressure to give a residue, which was purified by RP-HPLC to give the title compound (153 mg, 56% yield) as a brown oil. [M+H]⁺=281.0. ¹H NMR (400 MHz, DMSO-d₆) δ ppm 8.66 (s, 1H), 7.76 (s, 1H), 7.41 (s, 1H), 6.96 (s, 1H), 4.44 (t, J=5.2 Hz, 1H), 3.84 (s, 3H), 3.44-3.35 (m, 2H), 1.65-1.55 (m, 2H), 1.48-1.41 (m, 2H), 1.17 (s, 6H).

4-((7-methoxyimidazo[1,2-a]pyridin-6-yl)sulfonyl)-4-methylpentan-1-ol: To a solution of 4-((7-methoxyimidazo[1,2-a]pyridin-6-yl)thio)-4-methylpentan-1-ol (50 mg, 160.49 μmol, 1 eq) in MeOH (1.5 mL) and H₂O (0.5 mL) was added Oxone® (592.00 mg, 962.96 μmol, 6 eq), and then the mixture was stirred at 30° C. for 2 hours. The crude title compound (50 mg) was used into the next step without work up or purification. [M+H]⁺=313.0.

4-((3-iodo-7-methoxyimidazo[1,2-a]pyridin-6-yl)sulfonyl)-4-methylpentan-1-ol: To a solution of 4-((7-methoxyimidazo[1,2-a]pyridin-6-yl)sulfonyl)-4-methylpentan-1-ol (50 mg, 160.06 μmol, 1 eq) in H₂O (0.2 mL) was added NIS (43.21 mg, 192.07 μmol, 1.2 eq). The mixture was stirred at 20° C. for 2 hours before it was quenched with saturated Na₂SO₃ (1 mL), and then extracted with EtOAc (3×5 mL). The combined organic layers were dried over Na₂SO₄, filtered and concentrated under reduced pressure to give a residue, which was purified by RP-HPLC to give the title compound (20 mg, 26% yield) as a light yellow solid. [M+H]⁺=438.9. ¹H NMR (400 MHz, DMSO-d₆) δ ppm 8.49 (s, 1H), 7.72 (s, 1H), 7.28 (s, 1H), 4.46 (t, J=5.2 Hz, 1H), 3.92 (s, 3H), 3.42-3.36 (m, 2H), 1.71-1.65 (m, 2H), 1.51-1.43 (m, 2H), 1.27 (s, 6H).

### Intermediate 5

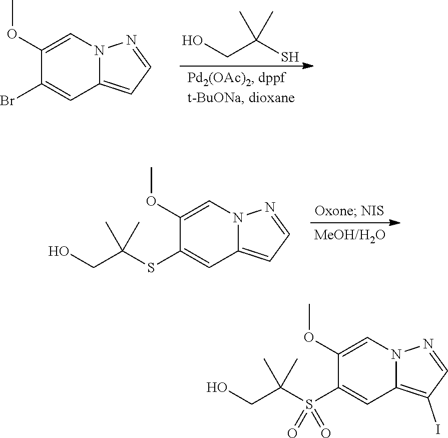

2-((6-methoxypyrazolo[1,5-a]pyridin-5-yl)thio)-2-methylpropan-1-ol: To a mixture of 5-bromo-6-methoxypyrazolo[1,5-a]pyridine (150 mg, 594.56 μmol, 1 eq), 2-mercapto-2-methylpropan-1-ol (126.27 mg, 1.19 mmol, 2 eq), dppf (65.92 mg, 118.91 μmol, 0.2 eq) and t-BuONa (114.28 mg, 1.19 mmol, 2 eq) in 1,4-dioxane (5 mL) was added Pd(OAc)₂ (13.35 mg, 59.46 μmol, 0.1 eq) under a N₂ atmosphere. The mixture was stirred at 100° C. for 16 hours before it was concentrated under reduced pressure, diluted with H₂O (30 mL) and extracted with EtOAc (3×50 mL). The combined organic layers were washed with H₂O (30 mL) and brine (30 mL), dried over Na₂SO₄, filtered and concentrated under reduced pressure to give a residue, which was purified by prep-TLC (SiO₂, 50% EtOAc/petroleum ether) to give the title compound (130 mg, 82% yield) as a yellow solid. [M+H]⁺=253.1. ¹H NMR (400 MHz, DMSO-d₆) δ ppm 8.41 (s, 1H), 7.95-7.88 (m, 2H), 6.59 (d, J=2.0 Hz, 1H), 4.84 (t, J=6.0 Hz, 1H), 3.85 (s, 3H), 3.31 (d, J=3.2 Hz, 2H), 1.18 (s, 6H).

2-((3-iodo-6-methoxypyrazolo[1,5-a]pyridin-5-yl)sulfonyl)-2-methylpropan-1-ol: To a mixture of 2-((6-methoxypyrazolo[1,5-a]pyridin-5-yl)thio)-2-methylpropan-1-ol (130 mg, 489.43 μmol, 1 eq) in MeOH (3 mL) and H₂O (1 mL) was added Oxone® (451.33 mg, 734.15 μmol, 1.5 eq). The mixture was stirred at 15° C. for 12 hours, and then H₂O (3 mL) and NIS (164.98 mg, 733.30 μmol, 1.5 eq) were added. The resulting mixture was stirred at 15° C. for 1 hour before it was quenched with saturated Na₂SO₃ (30 mL) and extracted with DCM (3×40 mL). The combined organic layers were dried over Na₂SO₄, filtered and concentrated under reduced pressure to give a residue, which was purified by column chromatography (SiO₂, 0-60% EtOAc/petroleum ether) to give the title compound (130 mg, 58% yield) as a yellow solid. [M+H]⁺=411.1. ¹H NMR (400 MHz, CDCl₃) δ ppm 8.25 (s, 1H), 8.15 (s, 1H), 8.02 (s, 1H), 4.00 (s, 3H), 3.78 (s, 2H), 1.42 (s, 6H).

### Intermediate 6

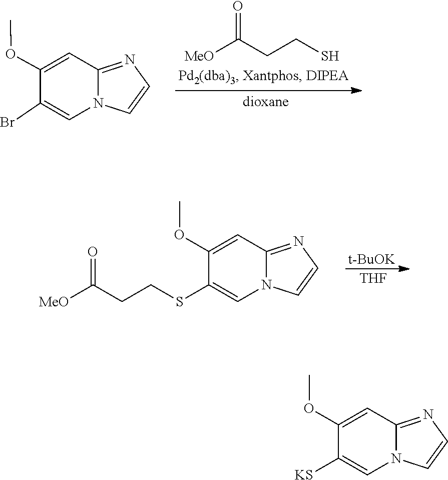

methyl 3-((7-methoxyimidazo[1,2-a]pyridin-6-yl)thio)propanoate: A mixture of 6-bromo-7-methoxyimidazo[1,2-a]pyridine (5 g, 19.82 mmol, 1 eq), methyl 3-mercaptopropanoate (2.36 mL, 21.80 mmol, 1.1 eq), Xantphos (2.29 g, 3.96 mmol, 0.2 eq) and DIPEA (6.90 mL, 39.64 mmol, 2 eq) in dioxane (90 mL) was degassed and purged with N₂ (3×), and then Pd₂(dba)₃ (1.81 g, 1.98 mmol, 0.1 eq) was added. The mixture was stirred at 90° C. for 12 h under a N₂ atmosphere before it was diluted with water (30 mL) and extracted with EtOAc (3×20 mL). The combined organic layers were dried over Na₂SO₄, filtered and concentrated in vacuo. The resulting crude material was purified by column chromatography (SiO₂, PE/EtOAc=5/1 to 0/1) to yield the title compound as a brown oil. ¹H NMR (400 MHz, CDCl₃) δ ppm 8.16 (s, 1H), 7.46 (t, J=1.6 Hz, 1H), 7.39 (s, 1H), 6.90 (s, 1H), 3.93 (s, 3H), 3.64 (s, 3H), 3.06 (t, J=7.2 Hz, 2H), 2.57 (t, J=7.2 Hz, 2H).

potassium 7-methoxyimidazo[1,2-a]pyridine-6-thiolate: To a solution of methyl 3-((7-methoxyimidazo[1,2-a]pyridin-6-yl)thio)propanoate (3.75 g, 12.67 mmol, 1 eq) in THF (60 mL) was added t-BuOK (1.99 g, 17.74 mmol, 1.4 eq) at 0° C. The mixture was stirred at 25° C. for 12 h before the precipitate was isolated by filtration. The filter cake was dried in vacuo to yield the title compound as a brown solid, which was used in the next step without further purification.

### Intermediate 7

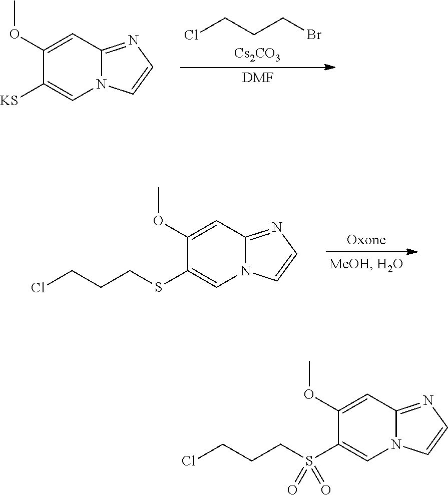

6-((3-chloropropyl)thio)-7-methoxyimidazo[1,2-a]pyridine: To a solution of potassium 7-methoxyimidazo[1,2-a]pyridine-6-thiolate (2.35 g, 10.76 mmol, 1 eq) in DMF (25 mL) was added Cs₂CO₃ (10.52 g, 32.29 mmol, 3 eq) and 1-bromo-3-chloropropane (1.27 mL, 12.92 mmol, 1.2 eq). The mixture was stirred at 80° C. for 4 h before it was diluted with H₂O (20 mL) and extracted with EtOAc (2×20 mL). The organic layer was dried over Na₂SO₄, filtered and concentrated in vacuo. The resulting crude material was purified by column chromatography (SiO₂, DCM/MeOH=100/1 to 10/1) to yield the title compound as a yellow oil. ¹H NMR (400 MHz, DMSO-d₆) δ ppm 8.57 (s, 1H), 7.71 (s, 1H), 7.39 (d, J=1.2 Hz, 1H), 6.98 (m, 1H), 3.88 (s, 3H), 3.75 (t, J=6.4 Hz, 2H), 2.94 (t, J=7.2 Hz, 2H), 2.00-1.90 (m, 2H).

6-((3-chloropropyl)sulfonyl)-7-methoxyimidazo[1,2-a]pyridine: To a solution of 6-((3-chloropropyl)thio)-7-methoxyimidazo[1,2-a]pyridine (1.92 g, 6.73 mmol, 1 eq) in MeOH (24 mL) and H₂O (8 mL) was added Oxone® (6.21 g, 10.10 mmol, 1.5 eq). The mixture was stirred at 25° C. for 12 h before it was filtered and concentrated in vacuo. The resulting crude material was purified via RP-HPLC to yield the title compound as a light yellow solid. ¹H NMR (400 MHz, DMSO-d₆) δ ppm 9.27 (s, 1H), 8.09 (s, 1H), 7.75 (d, J=1.6 Hz, 1H), 7.31 (s, 1H), 4.02 (s, 3H), 3.72 (t, J=6.4 Hz, 2H), 3.65-3.57 (m, 2H), 2.10-2.01 (m, 2H).

### Intermediate 8

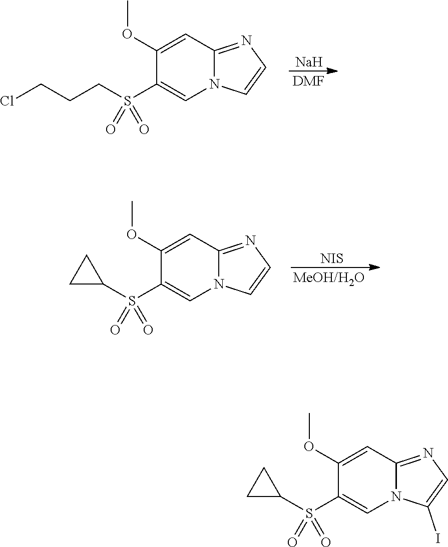

6-(cyclopropylsulfonyl)-7-methoxyimidazo[1,2-a]pyridine: To a solution of 6-((3-chloropropyl)sulfonyl)-7-methoxyimidazo[1,2-a]pyridine (238 mg, 741.82 μmol, 90% purity, 1 eq) in DMF (1 mL) was added NaH (59.34 mg, 1.48 mmol, 60% in mineral oil, 2 eq) at 0° C. The mixture was stirred at 0° C. for 0.5 hours before it was quenched with MeOH (1 mL). The mixture was purified by RP-HPLC to give the title compound (127 mg, 61% yield) as a brown solid. ¹H NMR (400 MHz, DMSO-d₆) δ ppm 9.07 (s, 1H), 7.96 (s, 1H), 7.54 (d, J=1.2 Hz, 1H), 7.19 (s, 1H), 3.98 (s, 3H), 3.13-2.99 (m, 1H), 1.20-1.02 (m, 4H).

6-(cyclopropylsulfonyl)-3-iodo-7-methoxyimidazo[1,2-a]pyridine: To a solution of 6-(cyclopropylsulfonyl)-7-methoxyimidazo[1,2-a]pyridine (127 mg, 453.05 μmol, 90% purity, 1 eq) in MeOH (3 mL) and H₂O (1 mL) was added NIS (122.32 mg, 543.66 μmol, 1.2 eq). The mixture was stirred at 25° C. for 1 hour before it was diluted with saturated NaHCO₃ (10 mL) and extracted with EtOAc (3×10 mL). The combined organic layers were dried over Na₂SO₄, filtered and concentrated under reduced pressure to give a residue. The residue was purified by column chromatography (SiO₂, petroleum ether/EtOAc=5/1 to 0/1) to give the title compound (107 mg, 56% yield) as a yellow solid. ¹H NMR (400 MHz, DMSO-d₆) δ ppm 8.49 (s, 1H), 7.71 (s, 1H), 7.31 (s, 1H), 4.01 (s, 3H), 3.17-3.05 (m, 1H), 1.22-1.15 (m, 2H), 1.14-1.05 (m, 2H).

### Intermediate 9

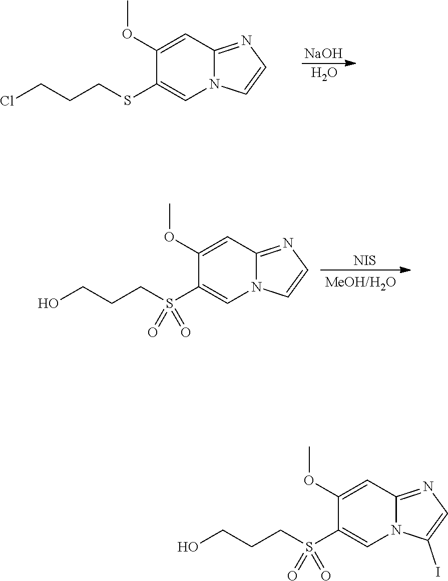

3-((7-methoxyimidazo[1,2-a]pyridin-6-yl)sulfonyl)propan-1-ol: To a solution of 6-((3-chloropropyl)sulfonyl)-7-methoxyimidazo[1,2-a]pyridine (50 mg, 155.84 μmol, 90% purity, 1 eq) in H₂O (1 mL) was added NaOH (6.23 mg, 155.84 μmol, 1 eq). The mixture was stirred at 25° C. for 1 hour before it was concentrated under reduced pressure to give a residue, which was purified by RP-HPLC to give the title compound (5 mg, 11% yield) as a yellow solid. [M+H]⁺=271.1. ¹H NMR (400 MHz, CDCl₃) δ ppm 8.78 (s, 1H), 7.62-7.55 (m, 2H), 7.02 (s, 1H), 4.01 (s, 3H), 3.78 (t, J=6.4 Hz, 2H), 3.56 (t, J=7.6 Hz, 2H), 2.07-2.01 (m, 2H).

3-((3-iodo-7-methoxyimidazo[1,2-a]pyridin-6-yl)sulfonyl)propan-1-ol: A mixture of 3-((7-methoxyimidazo[1,2-a]pyridin-6-yl)sulfonyl)propan-1-ol (5 mg, 16.65 μmol, 90% purity, 1 eq) and NIS (4.49 mg, 19.98 μmol, 1.2 eq) in MeOH (3 mL) and H₂O (1 mL) was stirred at 25° C. for 1 hour before it was diluted with water (10 mL) and extracted with EtOAc (3×10 mL). The combined organic layers were dried over Na₂SO₄, filtered and concentrated under reduced pressure to give the title compound (7 mg) as brown oil. [M+H]⁺=397.0.

### Intermediate 10

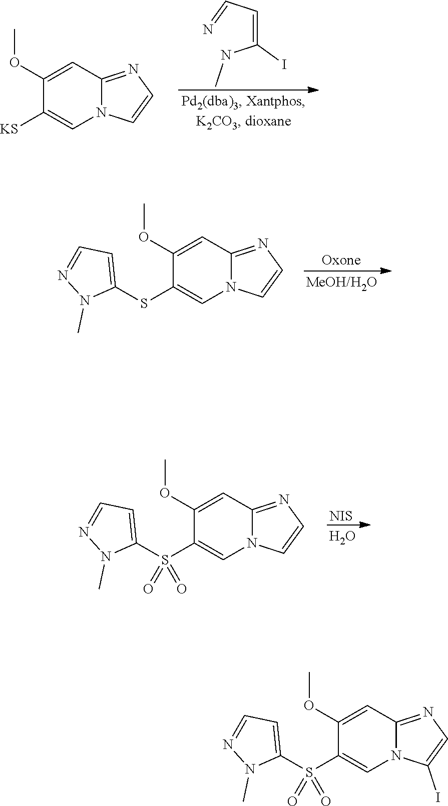

7-methoxy-6-((1-methyl-1H-pyrazol-5-yl)thio)imidazo[1,2-a]pyridine: A mixture of potassium 7-methoxyimidazo[1,2-a]pyridine-6-thiolate (250 mg, 1.15 mmol, 1 eq), 5-iodo-1-methyl-1H-pyrazole (166.73 mg, 801.59 μmol, 0.7 eq), Pd₂(dba)₃ (104.86 mg, 114.51 μmol, 0.1 eq), Xantphos (132.52 mg, 229.02 μmol, 0.2 eq) and K₂CO₃ (474.79 mg, 3.44 mmol, 3 eq) in dioxane (5 mL) was degassed and purged with N₂ 3 times, and then it was stirred at 80° C. for 2 hours under a N₂ atmosphere. The mixture was filtered and concentrated under reduced pressure to give a residue, which was purified by RP-HPLC to give the title compound (50 mg, 15% yield) as a gray solid. [M+H]⁺=261.1. ¹H NMR (400 MHz, DMSO-d₆) δ ppm 8.22 (s, 1H), 7.74 (s, 1H), 7.55 (s, 1H), 7.38 (s, 1H), 7.02 (s, 1H), 6.57 (d, J=1.6 Hz, 1H), 3.90 (s, 3H), 3.89 (s, 3H).

7-methoxy-6-((1-methyl-1H-pyrazol-5-yl)sulfonyl)imidazo[1,2-a]pyridine: A mixture of 7-methoxy-6-((1-methyl-1H-pyrazol-5-yl)thio)imidazo[1,2-a]pyridine (30.00 mg, 103.72 μmol, 1 eq) and Oxone® (191.29 mg, 311.16 μmol, 3 eq) in H₂O (0.4 mL) and MeOH (1.2 mL) was stirred at 30° C. for 24 hours to give the title compound (30 mg) as yellow liquid, which was used into the next step without work up and purification. [M+H]⁺=293.0.

3-iodo-7-methoxy-6-((1-methyl-1H-pyrazol-5-yl)sulfonyl)imidazo[1,2-a]pyridine: A mixture of 7-methoxy-6-((1-methyl-1H-pyrazol-5-yl)sulfonyl)imidazo[1,2-a]pyridine (30 mg, 102.63 μmol, 1 eq) and NIS (34.64 mg, 153.94 μmol, 1.5 eq) in H₂O (0.6 mL) was stirred at 20° C. for 2 hours before it was quenched with saturated Na₂SO₃ (1 mL). The mixture was concentrated under reduced pressure to give a residue, which was purified by column chromatography (SiO₂, petroleum ether/EtOAc=50/1 to 10/1) to give the title compound (45 mg, 94% yield) as a brown solid. [M+H]⁺=418.9. ¹H NMR (400 MHz, DMSO-d₆) δ ppm 8.76 (s, 1H), 7.74 (s, 1H), 7.64 (d, J=2.0 Hz, 1H), 7.25 (s, 1H), 7.08 (d, J=2.0 Hz, 1H), 4.04 (s, 3H), 3.86 (s, 3H).

### Intermediate 11

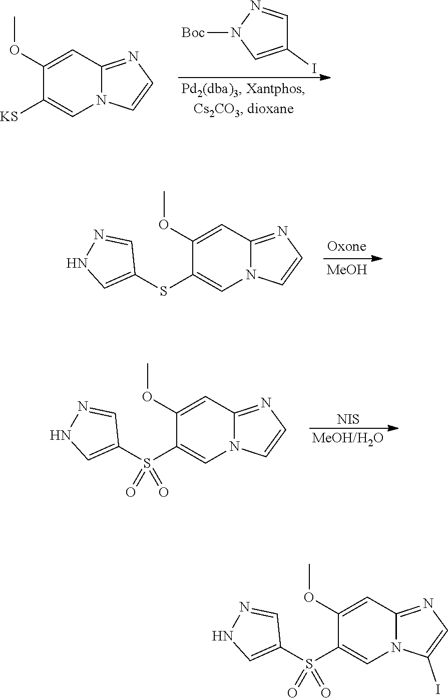

6-((1H-pyrazol-4-yl)thio)-7-methoxyimidazo[1,2-a]pyridine: A mixture of potassium 7-methoxyimidazo[1,2-a]pyridine-6-thiolate (800 mg, 3.66 mmol, 2.5 eq), tert-butyl 4-iodo-1H-pyrazole-1-carboxylate (431.06 mg, 1.47 mmol, 1 eq), Cs₂CO₃ (1.43 g, 4.40 mmol, 3 eq) and Xantphos (169.62 mg, 293.15 μmol, 0.2 eq) in 1, 4-dioxane (20 mL) was degassed and purged with N₂ 3 times, and then Pd₂(dba)₃ (134.22 mg, 146.58 μmol, 0.1 eq) was added. The mixture was stirred at 80° C. for 12 hours under a N₂ atmosphere before it was filtered and concentrated under reduced pressure to give a residue, which was purified by column chromatography (SiO₂, 0-8% MeOH/DCM) to give the title compound (120 mg, 30% yield) as a white solid. ¹H NMR (400 MHz, DMSO-d₆) δ ppm 13.34 (s, 1H), 8.10 (s, 1H), 7.81 (s, 1H), 7.79-7.60 (m, 2H), 7.33 (d, J=1.6 Hz, 1H), 6.97 (s, 1H), 3.90 (s, 3H).

6-((1H-pyrazol-4-yl)sulfonyl)-7-methoxyimidazo[1,2-a]pyridine: To a solution of 6-((1H-pyrazol-4-yl)thio)-7-methoxyimidazo[1,2-a]pyridine (120 mg, 438.51 μmol, 1 eq) in MeOH (6 mL) and H₂O (2 mL) was added Oxone® (404.37 mg, 657.77 μmol, 1.5 eq). The mixture was stirred at 25° C. for 12 hours to give the title compound (120 mg) as a white solid, which was used in the next step without further purification. [M+H]⁺=279.0.

6-((1H-pyrazol-4-yl)sulfonyl)-3-iodo-7-methoxyimidazo[1,2-a]pyridine: To a solution of 6-((1H-pyrazol-4-yl)sulfonyl)-7-methoxyimidazo[1,2-a]pyridine (120 mg, 431.21 μmol, 1 eq) in MeOH (9 mL) and H₂O (6 mL) was added NIS (145.52 mg, 646.81 μmol, 1.5 eq). The mixture was stirred at 25° C. for 3 hours before it was diluted with water (30 mL) and extracted with EtOAc (3×30 mL). The combined organic layers were washed with brine (50 mL), dried over Na₂SO₄, filtered and concentrated to give the residue, which was purified by column chromatography (SiO₂, 0-10% MeOH/DCM) to give the title compound (90 mg, 47% yield) as a white solid. [M+H]⁺=404.9. ¹H NMR (400 MHz, DMSO-d₆) δ ppm 13.77 (s, 1H), 8.71 (s, 1H), 8.56 (s, 1H), 8.02 (s, 1H), 7.69 (s, 1H), 7.19 (s, 1H), 3.92 (s, 3H).

### Intermediate 12

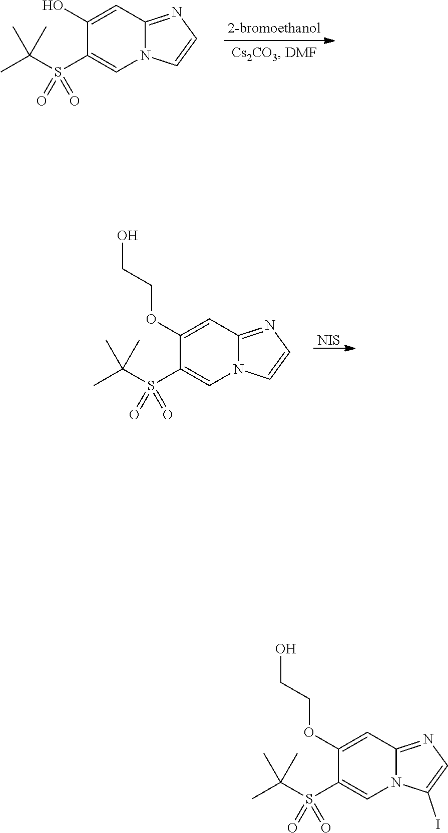

2-((6-(tert-butylsulfonyl)imidazo[1,2-a]pyridin-7-yl)oxy)ethan-1-ol: A mixture of 6-(tert-butylsulfonyl)imidazo[1,2-a]pyridin-7-ol (69.86 mg, 559.01 μmol, 39.62 μL, 1.5 eq), 2-bromoethanol (69.86 mg, 559.01 μmol, 39.62 μL, 1.5 eq), and Cs₂CO₃ (364.27 mg, 1.12 mmol, 3 eq) in DMF (2 mL) was degassed and purged with N₂ 3 times, and then the mixture was stirred at 80° C. for 2 hours under a N₂ atmosphere. The reaction mixture was diluted with H₂O (20 mL) and the resulting mixture was purified by RP-HPLC to yield the title compound (40 mg, 32% yield) as a brown solid. ¹H NMR (400 MHz, DMSO-d₆) δ ppm 9.12 (s, 1H), 7.99 (s, 1H), 7.54 (d, J=1.2 Hz, 1H), 7.15 (s, 1H), 4.78 (t, J=5.2 Hz, 1H), 4.13 (t, J=4.8 Hz, 2H), 3.80-3.70 (m, 2H), 1.32 (s, 9H).

2-((6-(tert-butylsulfonyl)-3-iodoimidazo[1,2-a]pyridin-7-yl)oxy)ethan-1-ol: To a solution of 2-((6-(tert-butylsulfonyl)imidazo[1,2-a]pyridin-7-yl)oxy)ethan-1-ol (35 mg, 105.58 μmol, 1 eq) in MeOH (1.5 mL) and H₂O (1.5 mL) was added NIS (28.50 mg, 126.69 μmol, 1.2 eq). The mixture was stirred at 25° C. for 12 hours before it was concentrated under reduced pressure to remove the MeOH. The residue was purified by RP-HPLC to yield the title compound (33 mg, 66% yield) as a yellow solid. ¹H NMR (400 MHz, CDCl₃) δ ppm 8.67 (s, 1H), 7.68 (s, 1H), 7.05 (s, 1H), 4.27 (t, J=4.4 Hz, 2H), 3.97 (d, J=2.8 Hz, 2H), 3.24-3.18 (m, 1H), 1.46 (s, 9H).

### Intermediate 13

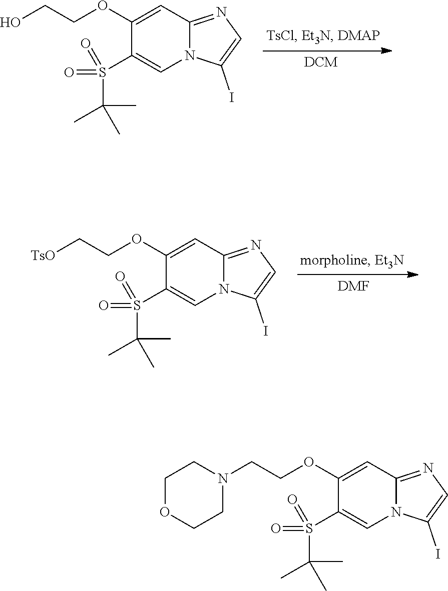

2-((6-(tert-butylsulfonyl)-3-iodoimidazo[1,2-a]pyridin-7-yl)oxy)ethyl 4-methylbenzenesulfonate: To a solution of 2-((6-(tert-butylsulfonyl)-3-iodoimidazo[1,2-a]pyridin-7-yl)oxy)ethan-1-ol (300 mg, 636.41 μmol, 1 eq) in DCM (3 mL) was added TsCl (89.78 mg, 1.27 mmol, 2 eq), DMAP (77.75 mg, 636.41 μmol, 1 eq) and Et₃N (193.19 mg, 1.91 mmol, 265.74 μL, 3 eq). The mixture was stirred at 25° C. for 12 hours before it was diluted with H₂O (10 mL), and extracted with DCM (3×20 mL). The combined organic layers were washed with brine (10 mL), dried over Na₂SO₄, filtered and concentrated under reduced pressure to give the title compound (220 mg) as a yellow solid.

4-(2-((6-(tert-butylsulfonyl)-3-iodoimidazo[1,2-a]pyridin-7-yl)oxy)ethyl)morpholine: A mixture of 2-((6-(tert-butylsulfonyl)-3-iodoimidazo[1,2-a]pyridin-7-yl)oxy)ethyl 4-methylbenzenesulfonate (60 mg, 103.73 μmol, 1 eq), morpholine (18.07 mg, 207.45 μmol, 18.26 μL, 2 eq) and Et₃N (31.49 mg, 311.18 μmol, 43.31 μL, 3 eq) in DMF (2 mL) was degassed and purged with N₂ 3 times, and then the mixture was stirred at 80° C. for 12 hours under a N₂ atmosphere. The reaction mixture was concentrated under reduced pressure to give a residue, which was purified by column chromatography (SiO₂, DCM/MeOH=1/0 to 9/1) to yield the title compound (50 mg, 64% yield) as a yellow solid. ¹H NMR (400 MHz, DMSO-d₆) δ ppm 8.50 (s, 1H), 7.72 (s, 1H), 7.30 (s, 1H), 4.25 (t, J=5.6 Hz, 2H), 3.56 (t, J=4.8 Hz, 4H), 3.38-3.29 (m, 4H), 2.72 (t, J=5.6 Hz, 2H), 1.34 (s, 9H).

### Intermediate 14

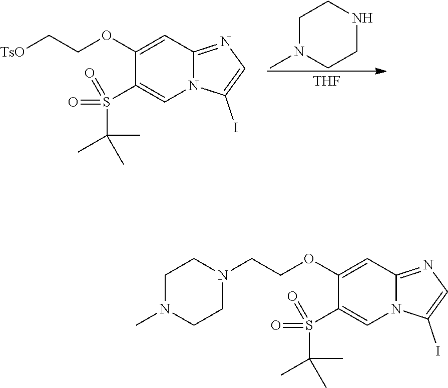

6-(tert-butylsulfonyl)-3-iodo-7-(2-(4-methylpiperazin-1-yl)ethoxy)imidazo[1,2-a]pyridine: To a solution of 2-((6-(tert-butylsulfonyl)-3-iodoimidazo[1,2-a]pyridin-7-yl)oxy)ethyl 4-methylbenzenesulfonate (50 mg, 77.80 μmol, 1 eq) in THF (3 mL) was added 1-methylpiperazine (38.96 mg, 388.98 μmol, 43.15 μL, 5 eq). The mixture was stirred at 60° C. for 12 hours before it was concentrated under reduced pressure to give a residue, which was purified by column chromatography (SiO₂, DCM/MeOH=1/0 to 0/1) to yield the title compound (25 mg, 51% yield) as a white solid. ¹H NMR (400 MHz, DMSO-d₆) δ ppm 8.49 (s, 1H), 7.72 (s, 1H), 7.30 (s, 1H), 4.23 (t, J=5.2 Hz, 2H), 2.77-2.71 (m, 2H), 2.64-2.54 (m, 4H), 2.31-2.23 (m, 7H), 1.34 (s, 9H).

### Intermediate 15

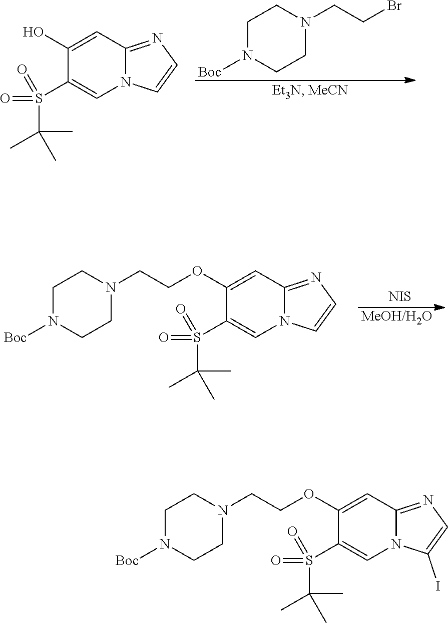

tert-butyl 4-(2-((6-(tert-butylsulfonyl)imidazo[1,2-a]pyridin-7-yl)oxy)ethyl)piperazine-1-carboxylate: To a solution of 6-(tert-butylsulfonyl)imidazo[1,2-a]pyridin-7-ol (150 mg, 530.86 μmol, 1 eq) in MeCN (5 mL) was added Et₃N (161.15 mg, 1.59 mmol, 221.67 μL, 3 eq) and tert-butyl 4-(2-bromoethyl)piperazine-1-carboxylate (186.78 mg, 637.03 μmol, 1.2 eq). The mixture was stirred at 80° C. for 3 hours before it was diluted with water (10 mL) and extracted with EtOAc (3×20 mL). The combined organic layers was washed with brine (20 mL), dried over Na₂SO₄, filtered and concentrated under reduced pressure to give a residue, which was purified by column chromatography (SiO₂, DCM/MeOH=1/0 to 9/1) to give the title compound (100 mg, 32% yield) as a brown solid. ¹H NMR (400 MHz, DMSO-d₆) δ ppm 9.12 (s, 1H), 7.99 (s, 1H), 7.54 (d, J=1.2 Hz, 1H), 7.16 (s, 1H), 4.21 (t, J=5.6 Hz, 2H), 3.31-3.21 (m, 4H), 2.78-2.70 (m, 2H), 2.49-2.42 (m, 4H), 1.39 (s, 9H), 1.32 (s, 9H).

tert-butyl 4-(2-((6-(tert-butylsulfonyl)-3-iodoimidazo[1,2-a]pyridin-7-yl)oxy)ethyl)piperazine-1-carboxylate: To a solution of tert-butyl 4-(2-((6-(tert-butylsulfonyl)imidazo[1,2-a]pyridin-7-yl)oxy)ethyl)piperazine-1-carboxylate (80 mg, 137.16 μmol, 1 eq) in MeOH (5 mL) and H₂O (5 mL) was added NIS (61.72 mg, 274.33 μmol, 2 eq). The mixture was stirred at 0° C. for 2 hours before it was diluted with water (10 mL) and extracted with EtOAc (3×20 mL). The combined organic layers were washed with brine (10 mL), dried over Na₂SO₄, filtered and concentrated under reduced pressure to give a residue, which was purified by column chromatography (SiO₂, DCM/MeOH=100/0 to 90/10) to give the title compound (50 mg, 49% yield) as a brown solid. ¹H NMR (400 MHz, DMSO-d₆) δ ppm 8.78 (s, 1H), 8.50 (s, 1H), 7.66 (s, 1H), 4.57 (t, J=6.4 Hz, 2H), 4.25 (s, 2H), 3.30-3.24 (m, 4H), 2.77-2.65 (m, 4H), 1.34 (s, 9H), 1.31 (s, 9H).

### Intermediate 16

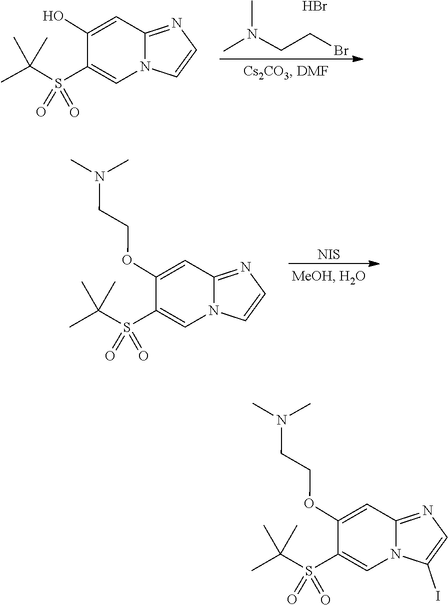

2-((6-(tert-butylsulfonyl)imidazo[1,2-a]pyridin-7-yl)oxy)-N,N-dimethylethan-1-amine: A mixture of 6-(tert-butylsulfonyl)imidazo[1,2-a]pyridin-7-ol (130 mg, 460.08 μmol, 1 eq), Cs₂CO₃ (749.51 mg, 2.30 mmol, 5 eq), and 2-bromo-N,N-dimethylethan-1-amine hydrobromide (214.35 mg, 920.16 μmol, 2 eq) in DMF (4 mL) was stirred at 80° C. for 12 hours under a N₂ atmosphere. The reaction mixture was filtered and concentrated under reduced pressure to give a residue, which was purified by column chromatography (SiO₂, DCM/MeOH=1/0 to 2/3) to give the title compound (50 mg, 30% yield) as a yellow solid. ¹H NMR (400 MHz, DMSO-d₆) δ ppm 9.13 (s, 1H), 7.99 (s, 1H), 7.55 (d, J=1.2 Hz, 1H), 7.18 (s, 1H), 4.20 (t, J=6.0 Hz, 2H), 2.74 (t, J=6.0 Hz, 2H), 2.30 (s, 6H), 1.32 (s, 9H).

2-((6-(tert-butylsulfonyl)-3-iodoimidazo[1,2-a]pyridin-7-yl)oxy)-N,N-dimethylethan-1-amine: To a mixture of 2-((6-(tert-butylsulfonyl)imidazo[1,2-a]pyridin-7-yl)oxy)-N,N-dimethylethan-1-amine (45 mg, 124.45 μmol, 1 eq) in MeOH (3 mL) and H₂O (1 mL) was added NIS (30.80 mg, 136.90 μmol, 1.1 eq) at 0° C., and the mixture was stirred at 0° C. for 2 hours under a N₂ atmosphere. The reaction mixture was concentrated under reduced pressure to give the title compound (50 mg, 80% yield) as yellow oil, which was used in the next step with further purification.

### Intermediate 17

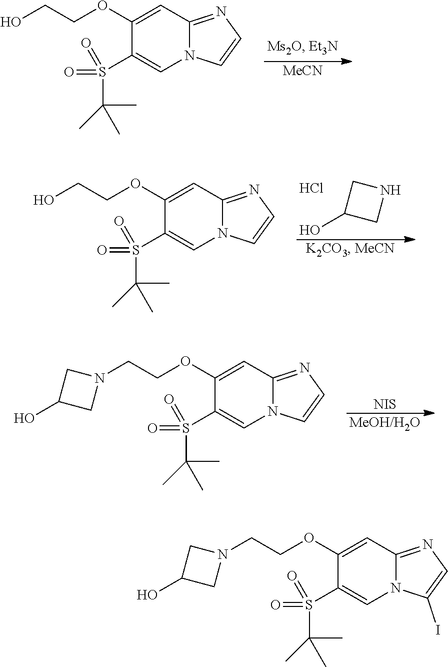

2-((6-(tert-butylsulfonyl)imidazo[1,2-a]pyridin-7-yl)oxy)ethyl methanesulfonate: To a solution of 2-((6-(tert-butylsulfonyl)imidazo[1,2-a]pyridin-7-yl)oxy)ethan-1-ol (150 mg, 452.48 μmol, 1 eq) in MeCN (3 mL) was added Ms₂O (157.64 mg, 904.96 μmol, 2 eq) and Et₃N (137.36 mg, 1.36 mmol, 188.94 μL, 3 eq). The mixture was stirred at 40° C. for 2 hours before it was filtered and concentrated under reduced pressure to give a residue, which was purified by column chromatography (SiO₂, DCM/MeOH=1/0 to 10/1) to give the title compound (100 mg, 53% yield) as a light yellow solid. ¹H NMR (400 MHz, DMSO-d₆) δ ppm 9.16 (s, 1H), 8.01 (d, J=0.8 Hz, 1H), 7.56 (d, J=1.2 Hz, 1H), 7.21 (s, 1H), 4.56-4.51 (m, 2H), 4.45-4.39 (m, 2H), 3.25 (s, 3H), 1.32 (s, 9H).

1-(2-((6-(tert-butylsulfonyl)imidazo[1,2-a]pyridin-7-yl)oxy)ethyl)azetidin-3-ol: A mixture of 2-((6-(tert-butylsulfonyl)imidazo[1,2-a]pyridin-7-yl)oxy)ethyl methanesulfonate (100 mg, 239.08 μmol, 1 eq), azetidin-3-ol hydrochloride (52.38 mg, 478.15 μmol, 2 eq), and K₂CO₃ (132.17 mg, 956.31 μmol, 4 eq) in MeCN (3 mL) was degassed and purged with N₂ 3 times, and then the mixture was stirred at 80° C. for 12 hours under a N₂ atmosphere. The reaction mixture was filtered and concentrated under reduced pressure to give a residue, which was purified by column chromatography (SiO₂, DCM/MeOH=1/0 to 3/1) to give the title compound (50 mg, 53% yield) as a light yellow solid. ¹H NMR (400 MHz, CD₃OD) δ ppm 9.05 (s, 1H), 7.84 (d, J=0.8 Hz, 1H), 7.52 (d, J=1.6 Hz, 1H), 7.03 (s, 1H), 4.38-4.30 (m, 1H), 4.17 (t, J=5.6 Hz, 2H), 3.91-3.82 (m, 2H), 3.24-3.20 (m, 2H), 3.06 (t, J=5.6 Hz, 2H), 1.37 (s, 9H).

1-(2-((6-(tert-butylsulfonyl)-3-iodoimidazo[1,2-a]pyridin-7-yl)oxy)ethyl)azetidin-3-ol: A mixture of 1-(2-((6-(tert-butylsulfonyl)imidazo[1,2-a]pyridin-7-yl)oxy)ethyl)azetidin-3-ol (35 mg, 89.13 μmol, 1 eq), and NIS (60.15 mg, 267.38 μmol, 3 eq) in MeOH (1 mL) and H₂O (0.2 mL) was stirred at 25° C. for 2 hours. The reaction mixture was quenched by the addition saturated Na₂SO₃ (5 mL) at 0° C., and then it was diluted with H₂O (10 mL) and extracted with EtOAc (3×30 mL). The combined organic layers were washed with brine (10 mL), dried over Na₂SO₄, filtered and concentrated under reduced pressure to give a residue, which was purified by column chromatography (SiO₂, DCM/MeOH=0/1 to 3/1) to give the title compound (30 mg, 63% yield) as a white solid. ¹H NMR (400 MHz, CD₃OD) δ ppm 8.69 (s, 1H), 7.67 (s, 1H), 7.16 (s, 1H), 4.48-4.34 (m, 1H), 4.22 (t, J=5.6 Hz, 2H), 3.91-3.81 (m, 2H), 3.24-3.17 (m, 2H), 3.07 (t, J=5.6 Hz, 2H), 1.42 (s, 9H).

### Intermediate 18

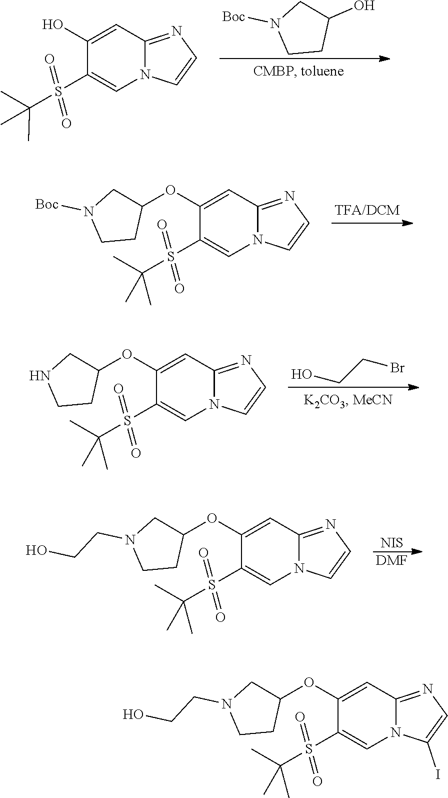

tert-butyl 3-((6-(tert-butylsulfonyl)imidazo[1,2-a]pyridin-7-yl)oxy)pyrrolidine-1-carboxylate: A mixture of 6-(tert-butylsulfonyl)imidazo[1,2-a]pyridin-7-ol (1.0 g, 3.54 mmol, 1 eq), tert-butyl 3-hydroxypyrrolidine-1-carboxylate (1.33 g, 7.08 mmol, 2.0 eq), and CMBP (2.56 g, 10.62 mmol, 3.0 eq) in toluene (20 mL) was degassed and purged with N₂ 3 times, and then the mixture was stirred at 110° C. for 12 hours under a N₂ atmosphere. The reaction mixture was filtered and concentrated to give a residue, which was purified by prep-TLC (SiO₂, DCM/MeOH=1/0 to 10/1) to give the title compound (1.5 g, crude) as red oil. [M+H]⁺=423.9.

6-(tert-butylsulfonyl)-7-(pyrrolidin-3-yloxy)imidazo[1,2-a]pyridine: A mixture of tert-butyl 3-((6-(tert-butylsulfonyl)imidazo[1,2-a]pyridin-7-yl)oxy)pyrrolidine-1-carboxylate (1.5 g, 3.54 mmol, 1 eq) in TFA (4 mL) and DCM (20 mL) was stirred at 25° C. for 12 hours. The reaction mixture was filtered and concentrated under reduced pressure to give a residue, which was purified by RP-HPLC and prep-TLC (SiO₂, DCM:MeOH:NH₃—H₂O=1:0:0 to 8:1:0.001) to give the title compound (180 mg, 14% yield) as yellow oil. ¹H NMR (400 MHz, CDCl₃) δ ppm 8.75 (s, 1H), 7.62 (d, J=1.2 Hz, 1H), 7.58-7.51 (m, 1H), 6.98 (s, 1H), 5.06-4.95 (m, 1H), 3.34-3.26 (m, 1H), 3.26-3.19 (m, 1H), 3.11-3.04 (m, 1H), 3.04-2.96 (m, 1H), 2.16-2.01 (m, 3H), 1.42 (s, 9H).

2-(3-((6-(tert-butylsulfonyl)imidazo[1,2-a]pyridin-7-yl)oxy)pyrrolidin-1-yl)ethan-1-ol: To a solution of 6-(tert-butylsulfonyl)-7-(pyrrolidin-3-yloxy)imidazo[1,2-a]pyridine (165 mg, 459.17 μmol, 1 eq) and 2-bromoethan-1-ol (68.86 mg, 551.00 μmol, 39.06 μL, 1.2 eq) in MeCN (8 mL) was added K₂CO₃ (126.92 mg, 918.34 μmol, 2.0 eq). The mixture was stirred at 80° C. for 12 hours before it was filtered and concentrated to give a residue, which was purified by prep-TLC (SiO₂, DCM:MeOH:NH₃—H₂O=1:0:0 to 8:1:0.001) to give the title compound (150 mg, 80% yield) as yellow oil. ¹H NMR (400 MHz, CDCl₃) δ ppm 8.80-8.66 (m, 1H), 7.64-7.60 (m, 1H), 7.56 (s, 1H), 6.90 (s, 1H), 5.08-4.89 (m, 1H), 3.78-3.60 (m, 2H), 3.38-3.18 (m, 1H), 3.12-2.97 (m, 2H), 2.89-2.81 (m, 3H), 2.5-2.11 (m, 4H), 1.43 (s, 9H).

2-(3-((6-(tert-butylsulfonyl)-3-iodoimidazo[1,2-a]pyridin-7-yl)oxy)pyrrolidin-1-yl)ethan-1-ol: To a solution of 2-(3-((6-(tert-butylsulfonyl)imidazo[1,2-a]pyridin-7-yl)oxy)pyrrolidin-1-yl)ethan-1-ol (50 mg, 122.46 μmol, 1 eq) in DMF (1.0 mL) was added NIS (27.55 mg, 122.46 μmol, 3 eq). The mixture was stirred at 25° C. for 4 hours before it was diluted with H₂O (20 mL) and extracted with EtOAc (3×20 mL). The combined organic layers were washed with brine (10 mL), dried over Na₂SO₄, filtered and concentrated under reduced pressure to give a residue, which was purified by prep-TLC (SiO₂, DCM:MeOH:NH₃—H₂O=1:0:0 to 8:1:0.001) to give the title compound (40 mg, 53% yield) as a yellow solid. ¹H NMR (400 MHz, CDCl₃) δ ppm 8.70 (s, 1H), 7.66 (s, 1H), 6.94 (s, 1H), 5.11-5.01 (m, 1H), 3.81-3.60 (m, 2H), 3.44-3.25 (m, 1H), 3.14-3.01 (m, 2H), 2.99-2.83 (m, 3H), 2.53-2.36 (m, 1H), 2.24-2.15 (m, 1H), 1.44 (s, 9H).

### Intermediate 19

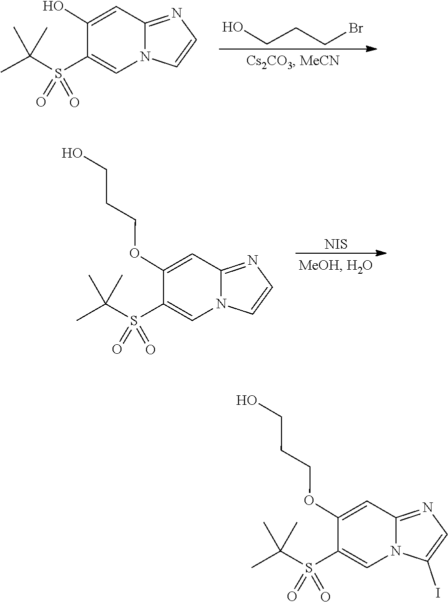

3-((6-(tert-butylsulfonyl)imidazo[1,2-a]pyridin-7-yl)oxy)propan-1-ol: To a solution of 6-(tert-butylsulfonyl)imidazo[1,2-a]pyridin-7-ol (100 mg, 353.91 μmol, 1 eq) and 3-bromopropan-1-ol (73.78 mg, 530.86 μmol, 48.01 μL, 1.5 eq) in MeCN (2 mL) was added Cs₂CO₃ (230.62 mg, 707.81 μmol, 2 eq). The mixture was stirred at 80° C. for 1 hour before it was filtered and concentrated under reduced pressure to give a residue, which was purified by prep-TLC (SiO₂, DCM:MeOH=10:1) to give the title compound (36 mg, 29% yield) as a light yellow solid. ¹H NMR (400 MHz, CDCl₃) δ ppm 8.75 (s, 1H), 7.64 (d, J=1.2 Hz, 1H), 7.58 (s, 1H), 7.12 (s, 1H), 4.30-4.25 (m, 2H), 3.93-3.89 (m, 2H), 2.15-2.11 (m, 2H), 1.43 (s, 9H).

3-((6-(tert-butylsulfonyl)-3-iodoimidazo[1,2-a]pyridin-7-yl)oxy)propan-1-ol: A mixture of 3-((6-(tert-butylsulfonyl)imidazo[1,2-a]pyridin-7-yl)oxy)propan-1-ol (36 mg, 103.72 μmol, 1 eq) and NIS (28.00 mg, 124.46 μmol, 1.2 eq) in MeOH (3 mL) and H₂O (1 mL) was degassed and purged with N₂ 3 times, and then the mixture was stirred at 25° C. for 1 hour under a N₂ atmosphere. The reaction mixture was filtered and concentrated under reduced pressure to give a residue, which was purified by column chromatography (SiO₂, petroleum ether/EtOAc=1/1 to 0/1) to give the title compound (50 mg, 99% yield) as a light yellow solid. [M+H]⁺=438.8. ¹H NMR (400 MHz, CDCl₃) δ ppm 8.68 (s, 1H), 7.66 (s, 1H), 7.13 (s, 1H), 4.33-4.28 (m, 2H), 3.92 (t, J=5.2, 2H), 2.19-2.11 (m, 2H), 1.45 (s, 9H).

### Intermediate 20

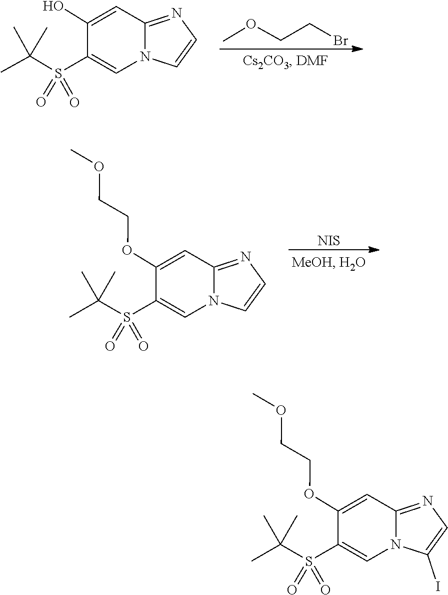

6-(tert-butylsulfonyl)-7-(2-methoxyethoxy)imidazo[1,2-a]pyridine: To a mixture of 6-(tert-butylsulfonyl)imidazo[1,2-a]pyridin-7-ol (50 mg, 176.95 μmol, 1 eq), and Cs₂CO₃ (172.96 mg, 530.86 μmol, 3 eq) in DMF (2 mL) was added 1-bromo-2-methoxyethane (29.51 mg, 212.34 μmol, 19.96 μL, 1.2 eq). The mixture was degassed and purged with N₂ 3 times, and then the mixture was stirred at 80° C. for 1 hour under a N₂ atmosphere. The reaction mixture was diluted with H₂O (10 mL) and extracted with EtOAc (3×10 mL), dried over Na₂SO₄, filtered and concentrated under reduced pressure to give the title compound (45 mg, 73% yield) as yellow oil. [M+H]⁺=313.0. ¹H NMR (400 MHz, CDCl₃) δ ppm 8.76 (s, 1H), 7.62 (d, J=1.2 Hz, 1H), 7.55 (s, 1H), 7.24-7.03 (m, 1H), 4.28-4.20 (m, 2H), 3.86-3.80 (m, 2H), 3.43 (s, 3H), 1.44 (s, 9H).

6-(tert-butylsulfonyl)-3-iodo-7-(2-methoxyethoxy)imidazo[1,2-a]pyridine: To a mixture of 6-(tert-butylsulfonyl)-7-(2-methoxyethoxy)imidazo[1,2-a]pyridine (45 mg, 129.65 μmol, 1 eq) in MeOH (3 mL) and H₂O (1 mL) was added NIS (32.09 mg, 142.61 μmol, 1.1 eq). The mixture was degassed and purged with N₂ 3 times, and then the mixture was stirred at 25° C. for 2 hours under a N₂ atmosphere. The reaction mixture was diluted with H₂O (10 mL) and extracted with DCM (3×20 mL), dried over Na₂SO₄, filtered and concentrated under reduced pressure to give the title compound (60 mg, 95% yield) as a yellow solid. [M+H]⁺=439.0. ¹H NMR (400 MHz, CDCl₃) δ ppm 8.72 (s, 1H), 7.69 (s, 1H), 7.07 (s, 1H), 4.28-4.20 (m, 2H), 3.88-3.80 (m, 2H), 3.43 (s, 3H), 1.45 (s, 9H).

### Intermediate 21

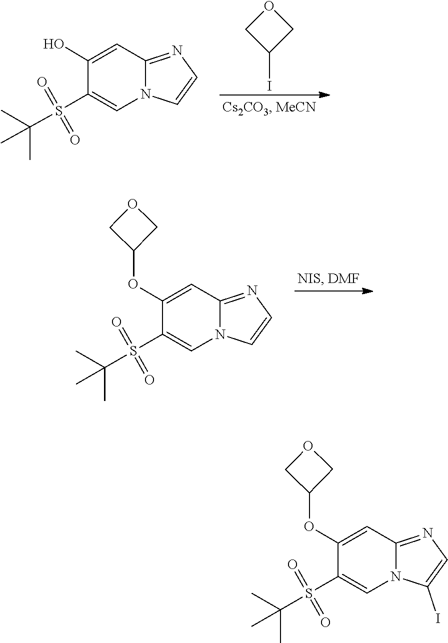 [SMILES: CC(C)(C)S(=O)(=O)c1cn2ccnc2cc1O ; IC1COC1 ; CC(C)(C)S(=O)(=O)c1cn2ccnc2cc1OC1COC1 ; CC(C)(C)S(=O)(=O)c1cn2c(I)cnc2cc1OC1COC1]

6-(tert-butylsulfonyl)-7-(oxetan-3-yloxy)imidazo[1,2-a]pyridine: To a solution of 6-(tert-butylsulfonyl)imidazo[1,2-a]pyridin-7-ol (300 mg, 1.06 mmol, 1 eq) in MeCN (8.0 mL) was added Cs₂CO₃ (691.86 mg, 2.12 mmol, 2.0 eq) and 3-iodooxetane (390.66 mg, 2.12 mmol, 2.0 eq). The mixture was stirred at 80° C. for 48 hours before it was diluted with H₂O (20 mL) and extracted with EtOAc (3×10 mL). The combined organic layers were washed with brine (10 mL), dried over Na₂SO₄, filtered and concentrated under reduced pressure to give a residue, which was purified by column chromatography (SiO₂, DCM/MeOH=1/0 to 10/1) to give the title compound (60 mg, 16% yield) as a yellow solid. ¹H NMR (400 MHz, DMSO-d₆) δ ppm 9.18 (s, 1H), 8.01 (s, 1H), 7.56 (s, 1H), 6.80 (s, 1H), 5.48-5.38 (m, 1H), 5.00 (t, J=6.8 Hz, 2H), 4.62-4.53 (m, 2H), 1.36 (s, 9H).

6-(tert-butylsulfonyl)-3-iodo-7-(oxetan-3-yloxy)imidazo[1,2-a]pyridine: A mixture of 6-(tert-butylsulfonyl)-7-(oxetan-3-yloxy)imidazo[1,2-a]pyridine (60 mg, 173.99 μmol, 1 eq), and NIS (46.97 mg, 208.78 μmol, 1.2 eq) in DMF (1.0 mL) was degassed and purged with N₂ 3 times, and then the mixture was stirred at 25° C. for 2 hours under a N₂ atmosphere. The reaction mixture was diluted with saturated Na₂SO₃ (20 mL) and filtered to give the title compound (30 mg, 36% yield) as a white solid, which was used in the next step without further purification. ¹H NMR (400 MHz, DMSO-d₆) δ ppm 8.53 (s, 1H), 7.74 (s, 1H), 6.95 (s, 1H), 5.51-5.42 (m, 1H), 5.01 (t, J=6.8 Hz, 2H), 4.64-4.51 (m, 2H), 1.38 (s, 9H).

### Intermediate 22

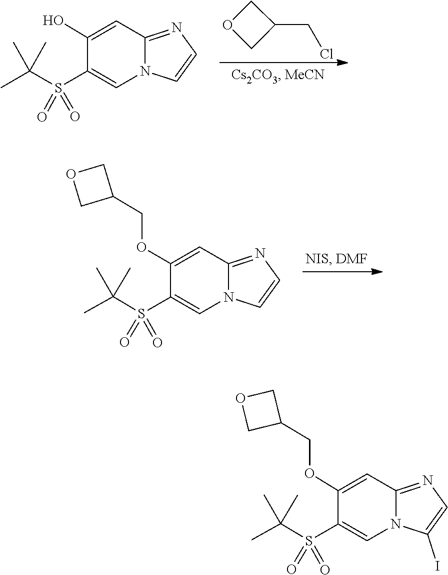

6-(tert-butylsulfonyl)-7-(oxetan-3-ylmethoxy)imidazo[1,2-a]pyridine: To a solution of 3-(chloromethyl)oxetane (37.71 mg, 353.91 μmol, 1 eq) and 6-(tert-butylsulfonyl)imidazo[1,2-a]pyridin-7-ol (100 mg, 353.91 μmol, 1 eq) in MeCN (1 mL) was added Cs₂CO₃ (345.93 mg, 1.06 mmol, 3 eq). The mixture was stirred at 80° C. for 12 hours before it was diluted with H₂O (10 mL) and extracted with DCM (3×10 mL). The combined organic layers were washed with brine (3×10 mL), dried over Na₂SO₄, filtered and concentrated under reduced pressure to give a residue, which was purified by column chromatography (SiO₂, DCM/MeOH=100/1 to 100/10) to give the title compound (20 mg, 16% yield) as a yellow solid. ¹H NMR (400 MHz, DMSO-d₆) δ ppm 9.14 (s, 1H), 8.00 (s, 1H), 7.56 (s, 1H), 7.20 (s, 1H), 4.74-4.65 (m, 2H), 4.47 (t, J=6.0 Hz, 2H), 4.34 (d, J=6.4 Hz, 2H), 3.48-3.37 (m, 1H), 1.31 (s, 9H).

6-(tert-butylsulfonyl)-3-iodo-7-(oxetan-3-ylmethoxy)imidazo[1,2-a]pyridine: To a solution of 6-(tert-butylsulfonyl)-7-(oxetan-3-ylmethoxy)imidazo[1,2-a]pyridine (17 mg, 47.16 μmol, 1 eq) in DMF (0.5 mL) was added NIS (15.92 mg, 70.75 μmol, 1.5 eq). The mixture was stirred at 25° C. for 1 hour before it was diluted with water (10 mL) and extracted with DCM (3×10 mL). The combined organic layers were washed with saturated Na₂SO₃ (4×10 mL), dried over Na₂SO₄, filtered and concentrated under reduced pressure to give a residue, which was purified by column chromatography (SiO₂, DCM/MeOH=100/1 to 95/5) to give the title compound (22 mg, 93% yield) as a yellow solid. ¹H NMR 400 MHz, DMSO-d₆) δ ppm 8.50 (s, 1H), 7.73 (s, 1H), 7.34 (s, 1H), 4.70 (dd, J=2.0, 6.0 Hz, 2H), 4.47 (t, J=6.0 Hz, 2H), 4.37 (d, J=6.4 Hz, 2H), 3.47-3.40 (m, 1H), 1.32 (s, 9H).

### Intermediate 23

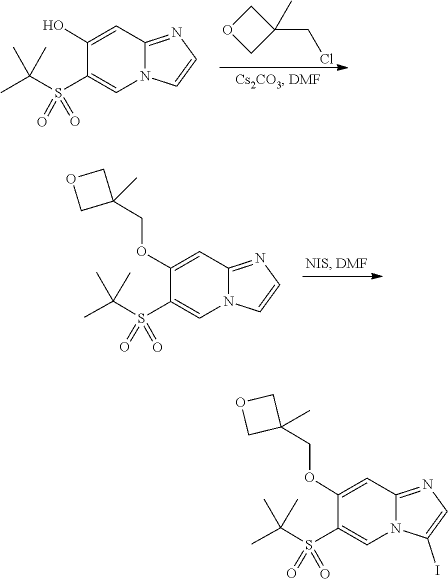

6-(tert-butylsulfonyl)-7-((3-methyloxetan-3-yl)methoxy)imidazo[1,2-a]pyridine: To a solution of 6-(tert-butylsulfonyl)imidazo[1,2-a]pyridin-7-ol (0.2 g, 707.81 μmol, 1 eq) in DMF (6 mL) was added Cs₂CO₃ (691.86 mg, 2.12 mmol, 3 eq) and 3-(chloromethyl)-3-methyloxetane (93.88 mg, 778.59 μmol, 1.1 eq). The mixture was stirred at 80° C. for 2 hours under a N₂ atmosphere before it was quenched with water (5 mL) at 25° C., then the reaction mixture was diluted with H₂O (2 mL) and extracted with EtOAc (3×20 mL). The combined organic layers were washed with brine (30 mL), dried over Na₂SO₄, filtered and concentrated under reduced pressure to give a residue, which was purified by column chromatography (SiO₂, petroleum ether/EtOAc=100/1 to 2/1) to give the title compound (140 mg, 53% yield) as a yellow solid. [M+H]⁺=339.0. ¹H NMR (400 MHz, DMSO-d₆) δ ppm 9.17-9.12 (m, 1H), 8.00 (s, 1H), 7.56 (d, J=1.2 Hz, 1H), 7.20 (s, 1H), 4.55-4.49 (m, 2H), 4.33-4.27 (m, 2H), 4.22 (s, 2H), 1.42 (s, 3H), 1.31 (s, 9H).

6-(tert-butylsulfonyl)-3-iodo-7-((3-methyloxetan-3-yl)methoxy)imidazo[1,2-a]pyridine: To a solution of 6-(tert-butylsulfonyl)-7-((3-methyloxetan-3-yl)methoxy)imidazo[1,2-a]pyridine (125 mg, 332.43 μmol, 1 eq) in DMF (1.5 mL) was added NIS (112.19 mg, 498.64 μmol, 1.5 eq) at 0° C. The mixture was stirred at 0° C. for 2 hours under a N₂ atmosphere before it was quenched with saturated NaHCO₃ (5 mL) at 25° C., then the reaction mixture was diluted with H₂O (2 mL) and extracted with DCM (3×10 mL). The combined organic layers were dried over Na₂SO₄, filtered and concentrated under reduced pressure to give a residue, which was purified by column chromatography (SiO₂, DCM/MeOH=100/1 to 10/1) to give the title compound (0.16 g, 93% yield) as a yellow solid. ¹H NMR (400 MHz, DMSO-d₆) δ ppm 8.50 (s, 1H), 7.73 (s, 1H), 7.34 (s, 1H), 4.51 (d, J=5.6 Hz, 2H), 4.31 (d, J=5.6 Hz, 2H), 4.26 (s, 2H), 1.43 (s, 3H), 1.32 (s, 9H).

### Intermediate 24

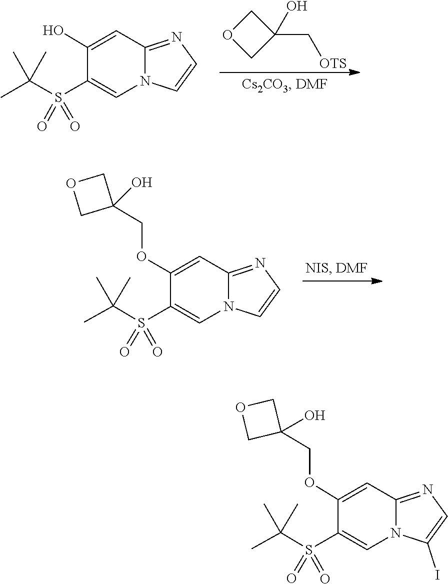

3-(((6-(tert-butylsulfonyl)imidazo[1,2-a]pyridin-7-yl)oxy)methyl)oxetan-3-01: To a solution of (3-hydroxyoxetan-3-yl)methyl 4-methylbenzenesulfonate (457.05 mg, 1.59 mmol, 1.5 eq) in DMF (1 mL) was added Cs₂CO₃ (1.04 g, 3.19 mmol, 3 eq) and 6-(tert-butylsulfonyl)imidazo[1,2-a]pyridin-7-ol (300 mg, 1.06 mmol, 1 eq). The mixture was stirred at 80° C. for 2 hours before it was partitioned between EtOAc (30 mL) and H₂O (10 mL). The organic phase was separated, washed with brine (3×10 mL), dried over Na₂SO₄, filtered and concentrated under reduced pressure to give a residue, which was purified by column chromatography (SiO₂, DCM/MeOH=100/0 to 91/9) to give the title compound (50 mg, 12% yield) as a yellow solid. ¹H NMR (400 MHz, DMSO-d₆) δ ppm 9.15 (s, 1H), 8.01 (s, 1H), 7.56 (s, 1H), 7.22 (s, 1H), 5.96 (s, 1H), 4.59 (d, J=6.8 Hz, 2H), 4.48 (d, J=6.4 Hz, 2H), 4.25 (s, 2H), 1.32 (s, 9H).

3-(((6-(tert-butylsulfonyl)-3-iodoimidazo[1,2-a]pyridin-7-yl)oxy)methyl)oxetan-3-ol: To a solution of 3-(((6-(tert-butylsulfonyl)imidazo[1,2-a]pyridin-7-yl)oxy)methyl)oxetan-3-ol (50 mg, 132.20 μmol, 1 eq) in DMF (1 mL) was added NIS (29.74 mg, 132.20 μmol, 1 eq) at 0° C. The mixture was stirred at 25° C. for 1 hour before it was partitioned between EtOAc (20 mL) and H₂O (10 mL). The organic phase was separated, washed with brine (3×10 mL), dried over Na₂SO₄, filtered and concentrated under reduced pressure to give the title compound (45 mg, 66% yield) as a brown solid. ¹H NMR (400 MHz, DMSO-d₆) δ ppm 8.51 (s, 1H), 7.74 (s, 1H), 7.36 (s, 1H), 6.01 (s, 1H), 4.58 (d, J=6.8 Hz, 2H), 4.49 (d, J=6.4 Hz, 2H), 4.29 (s, 2H), 1.34 (s, 9H).

### Intermediate 25

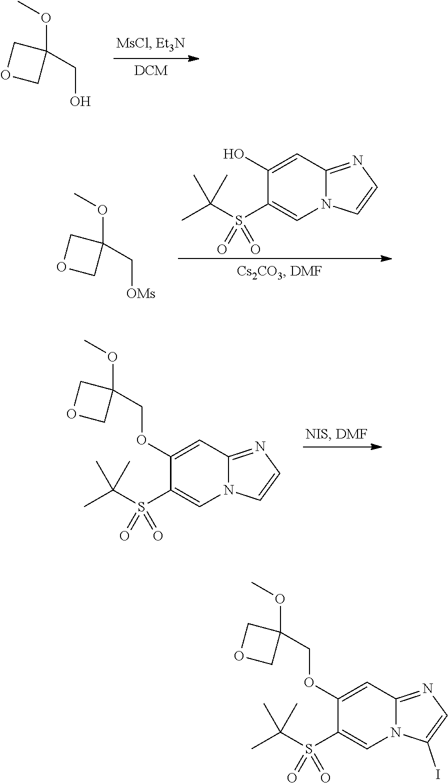

(3-methoxyoxetan-3-yl)methyl methanesulfonate: To a solution of (3-methoxyoxetan-3-yl)methanol (500 mg, 4.23 mmol, 1 eq) in DCM (10 mL) was added Et₃N (1.07 g, 10.58 mmol, 1.47 mL, 2.5 eq) and MsCl (727.27 mg, 6.35 mmol, 491.40 μL, 1.5 eq) at 0° C. The mixture was stirred at 25° C. for 12 hours before it was diluted with sat. NaHCO₃ (50 mL) at 0° C. and extracted with DCM (3×50 mL). The combined organic layers were dried over Na₂SO₄, filtered and concentrated under reduced pressure to give the title compound (850 mg) as yellow oil, which was used in the next step without further purification. ¹H NMR (400 MHz, CDCl₃) δ ppm 4.75 (d, J=7.2 Hz, 2H), 4.55 (s, 2H), 4.44 (d, J=7.2 Hz, 2H), 3.40 (s, 3H), 3.08 (s, 3H).

6-(tert-butylsulfonyl)-7-((3-methoxyoxetan-3-yl)methoxy)imidazo[1,2-a]pyridine: To a solution of (3-methoxyoxetan-3-yl)methyl methanesulfonate (850 mg, 4.33 mmol, 1 eq) in DMF (15 mL) was added Cs₂CO₃ (4.23 g, 13.00 mmol, 3 eq) and 6-(tert-butylsulfonyl)imidazo[1,2-a]pyridin-7-ol (244.80 mg, 866.37 μmol, 0.2 eq). The mixture was stirred at 60° C. for 2 hours before it was diluted with H₂O (20 mL) and extracted with EtOAc (3×20 mL). The combined organic layers were dried over Na₂SO₄, filtered and concentrated under reduced pressure to give a residue, which was purified by column chromatography (SiO₂, DCM/MeOH=100/1 to 95/5) to yield the title compound (200 mg, 12% yield) as a yellow solid. ¹H NMR (400 MHz, DMSO-d₆) δ ppm 9.15 (s, 1H), 8.01 (d, J=0.4 Hz, 1H), 7.56 (s, 1H), 7.27 (s, 1H), 4.61-4.57 (m, 2H), 4.55-4.52 (m, 2H), 4.43 (s, 2H), 3.29 (s, 3H), 1.31 (s, 9H).

6-(tert-butylsulfonyl)-3-iodo-7-((3-methoxyoxetan-3-yl)methoxy)imidazo[1,2-a]pyridine: To a solution of 6-(tert-butylsulfonyl)-7-((3-methoxyoxetan-3-yl)methoxy)imidazo[1,2-a]pyridine (200 mg, 507.87 μmol, 1 eq) in DMF (5 mL) was added NIS (171.39 mg, 761.81 μmol, 1.5 eq). The mixture was stirred at 25° C. for 2 hours before it was quenched with saturated Na₂SO₃ (5 mL) and extracted with EtOAc (3×50 mL). The combined organic layers were dried over Na₂SO₄, filtered and concentrated under reduced pressure to give the title compound (100 mg) as a yellow solid, which was used in the next step without further purification. ¹H NMR (400 MHz, DMSO-d₆) δ ppm 8.51 (s, 1H), 7.74 (s, 1H), 7.41 (s, 1H), 4.62-4.58 (m, 2H), 4.54-4.51 (m, 2H), 4.47 (s, 2H), 3.29 (s, 3H), 1.33 (s, 9H).

### Intermediate 26

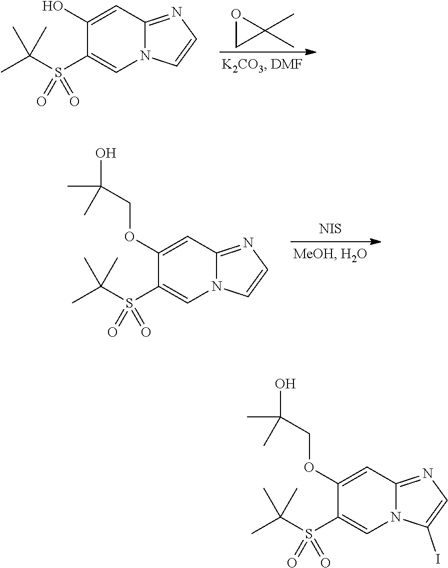

1-((6-(tert-butylsulfonyl)imidazo[1,2-a]pyridin-7-yl)oxy)-2-methylpropan-2-ol: To a solution of 6-(tert-butylsulfonyl)imidazo[1,2-a]pyridin-7-ol (200 mg, 707.81 μmol, 1 eq) in DMF (4 mL) was added K₂CO₃ (293.47 mg, 2.12 mmol, 3 eq) and 2,2-dimethyloxirane (61.24 mg, 849.37 μmol, 75.42 μL, 1.2 eq). The mixture was stirred at 100° C. for 12 hours before it was concentrated under reduced pressure to give a residue, which was purified by column chromatography (SiO₂, DCM/MeOH=100/1 to 20/1) to give the title compound (17 mg, 7% yield) as a yellow solid. [M+H]⁺=327.0. ¹H NMR (400 MHz, DMSO-d₆) δ ppm 9.14 (s, 1H), 7.99 (d, J=0.8 Hz, 1H), 7.54 (d, J=1.6 Hz, 1H), 7.11 (s, 1H), 4.51 (s, 1H), 3.86 (s, 2H), 1.33 (s, 9H), 1.25 (s, 6H).

1-((6-(tert-butylsulfonyl)-3-iodoimidazo[1,2-a]pyridin-7-yl)oxy)-2-methylpropan-2-ol: To a solution of 1-((6-(tert-butylsulfonyl)imidazo[1,2-a]pyridin-7-yl)oxy)-2-methylpropan-2-ol (30 mg, 91.91 μmol, 1 eq) in MeOH (2 mL) and H₂O (0.5 mL) was added NIS (31.02 mg, 137.86 μmol, 1.5 eq). The mixture was stirred at 25° C. for 2 hours before it was quenched with satd. Na₂SO₃ (5 mL) at 0° C., and then extracted with EtOAc (3×20 mL). The combined organic layers were washed with satd. NaHCO₃ (20 mL), dried over Na₂SO₄, filtered and concentrated under reduced pressure to give the title compound (30 mg, 65% yield) as a yellow solid, which was used in the next step without purification. [M+H]⁺=452.8. ¹H NMR (400 MHz, DMSO-d₆) δ ppm 8.49 (s, 1H), 7.72 (s, 1H), 7.25 (s, 1H), 4.54 (s, 1H), 3.89 (s, 2H), 1.34 (s, 9H), 1.25 (s, 6H).

### Intermediate 27

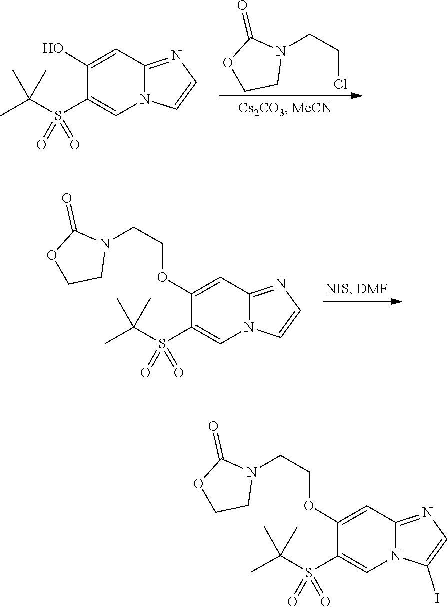

3-(2-((6-(tert-butylsulfonyl)imidazo[1,2-a]pyridin-7-yl)oxy)ethyl)oxazolidin-2-one: To a solution of 3-(2-chloroethyl)oxazolidin-2-one (150 mg, 902.56 μmol, 1 eq) and 6-(tert-butylsulfonyl)imidazo[1,2-a]pyridin-7-ol (255.03 mg, 902.56 μmol, 1 eq) in MeCN (10 mL) was added Cs₂CO₃ (588.14 mg, 1.81 mmol, 2.0 eq). The mixture was stirred at 80° C. for 12 hours before it was diluted with H₂O (20 mL) and extracted with EtOAc (3×20 mL). The combined organic layers were dried over Na₂SO₄, filtered and concentrated under reduced pressure to give a residue, which was purified by column chromatography (SiO₂, DCM:MeOH=1/0 to 10/1) to give the title compound (50 mg, 15% yield) as yellow oil. [M+H]⁺=368.1.

3-(2-((6-(tert-butylsulfonyl)-3-iodoimidazo[1,2-a]pyridin-7-yl)oxy)ethyl)oxazolidin-2-one: To a solution of 3-(2-((6-(tert-butylsulfonyl)imidazo[1,2-a]pyridin-7-yl)oxy)ethyl)oxazolidin-2-one (40 mg, 108.87 μmol, 1 eq) in DMF (1.0 mL) was added NIS (19.59 mg, 87.09 μmol, 0.8 eq). The mixture was stirred at 25° C. for 2 hours before it was concentrated under reduced pressure to give a residue, which was purified by column chromatography (SiO₂, DCM:MeOH=1/0 to 10/1) to give the title compound (30 mg, 25% yield) as yellow oil. ¹H NMR (400 MHz, DMSO-d₆) δ ppm 8.50 (s, 1H), 7.74 (s, 1H), 7.35 (s, 1H), 4.30 (t, J=5.2 Hz, 2H), 4.28-4.22 (m, 2H), 3.74-3.68 (m, 2H), 3.60-3.56 (m, 2H), 1.33 (s, 9H).

### Intermediate 28

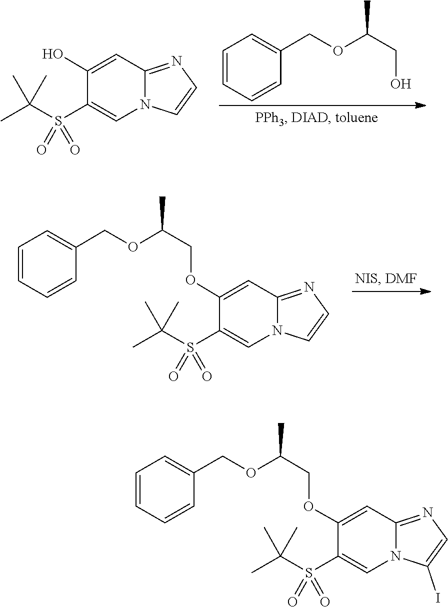

(S)-1-((6-(tert-butylsulfonyl)imidazo[1,2-a]pyridin-7-yl)oxy)propan-2-ol: To a solution of 6-(tert-butylsulfonyl)imidazo[1,2-a]pyridin-7-ol (200 mg, 707.81 μmol, 1 eq) and (S)-2-(benzyloxy)propan-1-ol (352.95 mg, 2.12 mmol, 3 eq) in toluene (1 mL) was added PPh₃ (371.30 mg, 1.42 mmol, 2 eq) and DIAD (286.25 mg, 1.42 mmol, 274.45 μL, 2 eq). The mixture was stirred at 20° C. for 2 hours before it was diluted with H₂O (20 mL) and extracted with EtOAc (3×10 mL). The combined organic layers were washed with brine (10 mL), dried over Na₂SO₄, filtered and concentrated under reduced pressure to give a residue, which was purified by column chromatography (SiO₂, petroleum ether/EtOAc=10/1 to 0/1) to give the title compound (80 mg, 27% yield) as a yellow solid. [M+H]⁺=403.1.

(S)-1-((6-(tert-butylsulfonyl)-3-iodoimidazo[1,2-a]pyridin-7-yl)oxy)propan-2-ol: To a solution of (S)-7-(2-(benzyloxy)propoxy)-6-(tert-butylsulfonyl)imidazo[1,2-a]pyridine (70 mg, 165.21 μmol, 1 eq) in DMF (1 mL) was added NIS (40.89 mg, 181.74 μmol, 1.1 eq). The mixture was stirred at 20° C. for 1 hour before it was diluted with H₂O (20 mL) and extracted with EtOAc (3×10 mL). The combined organic layers were washed with brine (10 mL), dried over Na₂SO₄, filtered and concentrated under reduced pressure to give a residue, which was purified by column chromatography (SiO₂, petroleum ether/EtOAc=10/1 to 0/1) to give the title compound (30 mg, 31% yield) as a white solid. ¹H NMR (400 MHz, DMSO-d₆) δ ppm 8.50 (s, 1H), 7.73 (s, 1H), 7.64-7.53 (m, 1H), 7.38-7.22 (m, 5H), 4.65-4.57 (m, 2H), 4.18 (d, J=4.4 Hz, 2H), 3.98-3.88 (m, 1H), 1.32-1.26 (m, 12H).

### Intermediate 29

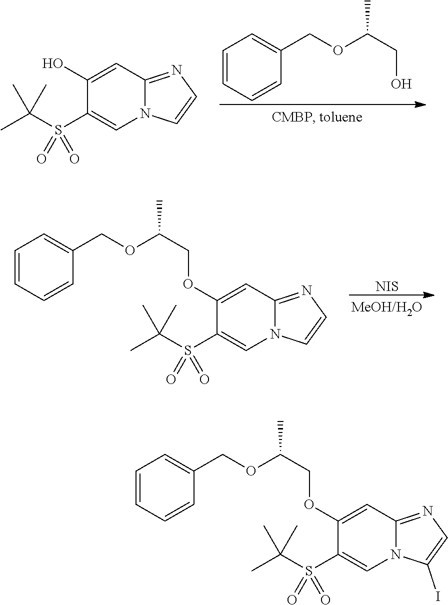

(R)-1-((6-(tert-butylsulfonyl)imidazo[1,2-a]pyridin-7-yl)oxy)propan-2-ol: To a solution of 6-(tert-butylsulfonyl)imidazo[1,2-a]pyridin-7-ol (200 mg, 707.81 μmol, 1 eq) and (R)-2-(benzyloxy)propan-1-ol (352.95 mg, 2.12 mmol, 3 eq) in toluene (5 mL) was added CMBP (1.02 g, 4.25 mmol, 6 eq). The mixture was stirred at 100° C. for 1 hour before it was filtered and concentrated under reduced pressure to give the residue, which was purified by column chromatography (SiO₂, EtOAc/petroleum ether 0 to 70%) to give the title compound (340 mg, 72% yield) as a brown solid. [M+H]⁺=403.0. ¹H NMR (400 MHz, DMSO-d₆) δ ppm 9.14 (s, 1H), 8.00 (s, 1H), 7.55 (d, J=1.2 Hz, 1H), 7.39-7.25 (m, 4H), 7.33-7.21 (m, 1H), 7.16 (s, 1H), 4.62 (s, 2H), 4.14 (d, J=4.0 Hz, 2H), 3.98-3.83 (m, 1H), 1.31-1.27 (m, 12H).

(R)-1-((6-(tert-butylsulfonyl)-3-iodoimidazo[1,2-a]pyridin-7-yl)oxy)propan-2-ol: To a solution of (R)-7-(2-(benzyloxy)propoxy)-6-(tert-butylsulfonyl)imidazo[1,2-a]pyridine (330 mg, 491.92 μmol, 1 eq) in methanol (5 mL) was added NIS (166.01 mg, 737.88 μmol, 1.5 eq) and H₂O (3 mL). The mixture was stirred at 25° C. for 1 hour before it was concentrated, diluted with water (30 mL) and extracted with EtOAc (3×30 mL). The combined organic layers were washed with brine (50 mL), dried over Na₂SO₄, filtered and concentrated to give a residue, which was purified by column chromatography (SiO₂, EtOAc/petroleum ether 0 to 70%) to give the title compound (170 mg, 59% yield) as a yellow solid. [M+H]⁺=528.9. ¹H NMR (400 MHz, DMSO-d₆) δ ppm 8.50 (s, 1H), 7.72 (s, 1H), 7.39-7.22 (m, 6H), 4.63-4.58 (m, 2H), 4.18 (d, J=4.8 Hz, 2H), 3.99-3.86 (m, 1H), 1.36-1.24 (m, 12H).

### Intermediate 30

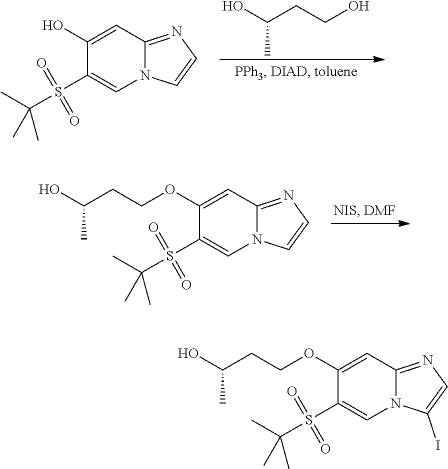

(S)-4-((6-(tert-butylsulfonyl)imidazo[1,2-a]pyridin-7-yl)oxy)butan-2-ol: To a solution of 6-(tert-butylsulfonyl)imidazo[1,2-a]pyridin-7-ol (100 mg, 353.91 μmol, 1 eq) in toluene (1 mL) was added (S)-butane-1,3-diol (95.68 mg, 1.06 mmol, 3 eq), PPh₃ (185.65 mg, 707.81 μmol, 2 eq) and DIAD (143.13 mg, 707.81 μmol, 137.22 μL, 2 eq). The mixture was stirred at 25° C. for 12 hours before it was filtered and concentrated under reduced pressure to give a residue, which was purified by column chromatography (SiO₂, 0-10% MeOH/DCM) to give the title compound (50 mg, 39% yield) as a black-brown solid. [M+H]⁺=327.0. ¹H NMR (400 MHz, DMSO-d₆) δ ppm 9.12 (s, 1H), 7.98 (s, 1H), 7.53 (d, J=1.2 Hz, 1H), 7.11 (s, 1H), 4.55 (d, J=4.4 Hz, 1H), 4.25-4.06 (m, 2H), 3.93-3.81 (m, 1H), 1.85-1.73 (m, 2H), 1.31 (s, 9H), 1.12 (d, J=6.0 Hz, 3H).

(S)-4-((6-(tert-butylsulfonyl)-3-iodoimidazo[1,2-a]pyridin-7-yl)oxy)butan-2-ol: To a solution of (S)-4-((6-(tert-butylsulfonyl)imidazo[1,2-a]pyridin-7-yl)oxy)butan-2-ol (50 mg, 137.86 μmol, 1 eq) in DMF (3 mL) was added NIS (31.02 mg, 137.86 μmol, 1 eq). The mixture was stirred at 25° C. for 2 hours before it was diluted with H₂O (10 mL) and extracted with EtOAc (2×10 mL). The combined organic layers were washed with brine (10 mL), dried over Na₂SO₄, filtered and concentrated under reduced pressure to give a residue, which was purified by column chromatography (SiO₂, DCM/MeOH=100/0 to 10/1) to give the title compound (30 mg, 43% yield) as a yellow solid. [M+H]⁺=452.8. ¹H NMR (400 MHz, CDCl₃) δ ppm 8.49 (s, 1H), 7.71 (s, 1H), 7.25 (s, 1H), 4.57 (d, J=4.8 Hz, 1H), 4.25-4.12 (m, 2H), 3.96-3.78 (m, 1H), 1.84-1.74 (m, 2H), 1.33 (s, 9H), 1.12 (d, J=6.0 Hz, 3H).

### Intermediate 31

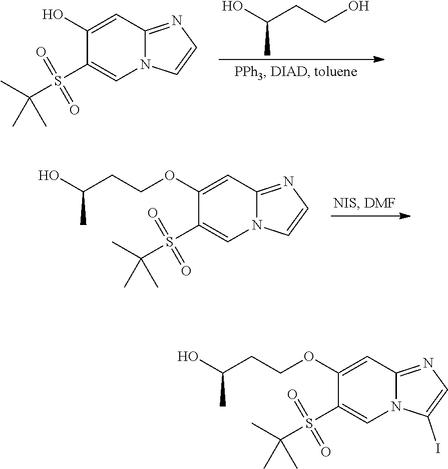

(R)-4-((6-(tert-butylsulfonyl)imidazo[1,2-a]pyridin-7-yl)oxy)butan-2-ol: To a solution of 6-(tert-butylsulfonyl)imidazo[1,2-a]pyridin-7-ol (300 mg, 1.06 mmol, 1 eq) in toluene (12 mL) was added DIAD (429.38 mg, 2.12 mmol, 411.67 μL, 2 eq), PPh₃ (556.95 mg, 2.12 mmol, 2 eq) and (R)-butane-1,3-diol (191.37 mg, 2.12 mmol, 2 eq). The mixture was stirred at 25° C. for 12 hours under a N₂ atmosphere. The mixture was filtered and the filtrate was concentrated to give the crude product, which was purified by prep-TLC (DCM/MeOH=10/1) to give the title compound (90 mg, 23% yield) as a yellow solid. ¹H NMR (400 MHz, DMSO-d₆) δ ppm 9.12 (s, 1H), 7.98 (s, 1H), 7.54 (d, J=1.2 Hz, 1H), 7.11 (s, 1H), 4.55 (d, J=4.8 Hz, 1H), 4.23-4.09 (m, 2H), 3.93-3.83 (m, 1H), 1.86-1.71 (m, 2H), 1.31 (s, 9H), 1.12 (d, J=6.4 Hz, 3H).

(R)-4-((6-(tert-butylsulfonyl)-3-iodoimidazo[1,2-a]pyridin-7-yl)oxy)butan-2-ol: To a solution of (R)-4-((6-(tert-butylsulfonyl)imidazo[1,2-a]pyridin-7-yl)oxy)butan-2-ol (90 mg, 248.15 μmol, 1 eq) in DMF (3 mL) was added NIS (83.74 mg, 372.23 μmol, 1.5 eq). The mixture was stirred at 25° C. for 3 hours before it was diluted with saturated Na₂SO₃ (20 mL) and extracted with DCM (3×20 mL). The combined organic layers were washed with saturated NaHCO₃ (20 mL), dried over Na₂SO₄, filtered and concentrated under reduced pressure to give a residue, which was purified by column chromatography (SiO₂, DCM/MeOH=100/1 to 10/1) to give the title compound (65 mg, 52% yield) as a yellow solid. ¹H NMR (400 MHz, DMSO-d₆) δ ppm 8.49 (s, 1H), 7.72 (s, 1H), 7.25 (s, 1H), 4.57 (d, J=4.8 Hz, 1H), 4.31-4.10 (m, 2H), 3.94-3.82 (m, 1H), 1.85-1.73 (m, 2H), 1.33 (s, 9H), 1.12 (d, J=6.0 Hz, 3H).

### Intermediate 32

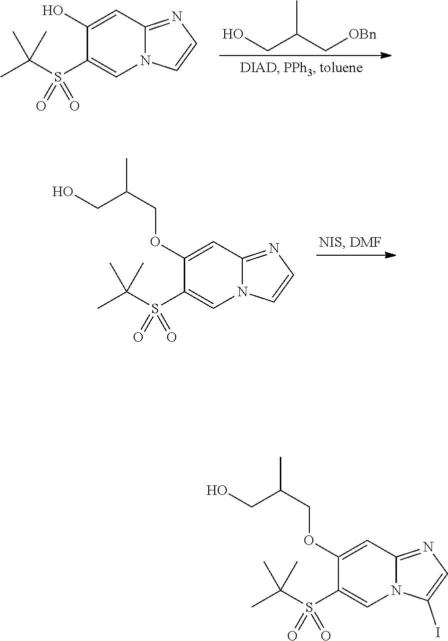

3-((6-(tert-butylsulfonyl)imidazo[1,2-a]pyridin-7-yl)oxy)-2-methylpropan-1-ol: To a solution of 6-(tert-butylsulfonyl)imidazo[1,2-a]pyridin-7-ol (200 mg, 786.46 μmol, 1 eq), 3-(benzyloxy)-2-methylpropan-1-ol (155.93 mg, 865.10 μmol, 1.1 eq) and PPh₃ (412.55 mg, 1.57 mmol, 2 eq) in toluene (5 mL) was added DIAD (318.06 mg, 1.57 mmol, 304.94 μL, 2 eq) and the mixture was stirred at 100° C. for 12 hours. Water (80 mL) was added and then the mixture was extracted with EtOAc (2×80 mL). The combined organic layers were washed with brine (2×80 mL), dried over anhydrous Na₂SO₄, filtered and concentrated under reduced pressure to give the crude product, which was purified by column chromatography (SiO₂, EtOAc/petroleum ether=0 to 1/0) to give the title compound (250 mg, 50% yield) as a brown solid. [M+H]⁺=417.1.

3-((6-(tert-butylsulfonyl)-3-iodoimidazo[1,2-a]pyridin-7-yl)oxy)-2-methylpropan-1-ol: The solution of 3-((6-(tert-butylsulfonyl)imidazo[1,2-a]pyridin-7-yl)oxy)-2-methylpropan-1-ol (240 mg, 374.52 μmol, 1 eq) and NIS (101.11 mg, 449.42 μmol, 1.2 eq) in DMF (2 mL) was stirred at 15° C. for 1 hour. The reaction mixture was concentrated under reduced pressure and the residue was purified by column chromatography (SiO₂, EtOAc/petroleum ether=0 to 1/1) to give the title compound (170 mg, 75% yield) as a yellow solid. ¹H NMR (400 MHz, CDCl₃) δ ppm 8.67 (s, 1H), 7.65 (s, 1H), 7.32 (d, J=4.4 Hz, 4H), 7.26-7.20 (m, 1H), 7.10 (s, 1H), 4.53 (s, 2H), 4.24-4.13 (m, 1H), 4.10-3.99 (m, 1H), 3.57 (d, J=6.4 Hz, 2H), 2.42-2.31 (m, 1H), 1.41 (s, 9H), 1.16 (d, J=6.8 Hz, 3H).

### Intermediate 33

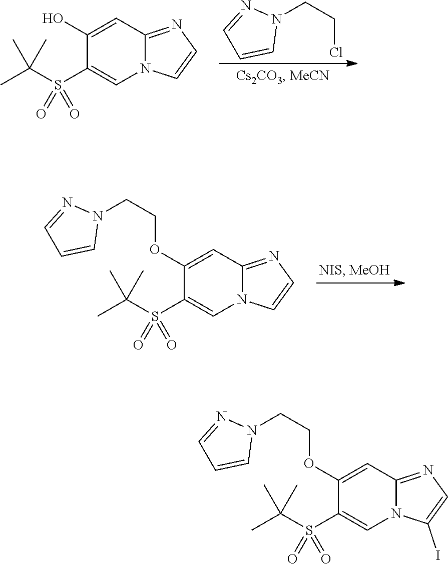

7-(2-(1H-pyrazol-1-yl)ethoxy)-6-(tert-butylsulfonyl)imidazo[1,2-a]pyridine: To a solution of 1-(2-chloroethyl)-1H-pyrazole (90.11 mg, 690.12 μmol, 1.3 eq) and 6-(tert-butylsulfonyl)imidazo[1,2-a]pyridin-7-ol (150 mg, 530.86 μmol, 1 eq) in MeCN (2 mL) was added Cs₂CO₃ (518.89 mg, 1.59 mmol, 3 eq). The mixture was stirred at 80° C. for 12 hours before it was filtered and concentrated under reduced pressure. The residue was purified by column chromatography (SiO₂, DCM:MeOH=1:0 to 10:1) to give the title compound (80 mg) as a white solid that was used in the next step without further purification.

7-(2-(1H-pyrazol-1-yl)ethoxy)-6-(tert-butylsulfonyl)-3-iodoimidazo[1,2-a]pyridine: To a solution of 7-(2-(1H-pyrazol-1-yl)ethoxy)-6-(tert-butylsulfonyl)imidazo[1,2-a]pyridine (64.00 mg, 183.69 μmol, 1 eq) in MeOH (5 mL) was added NIS (82.65 mg, 367.37 μmol, 2 eq). The mixture was stirred at 0° C. for 2 hours before it was quenched with sat. Na₂SO₃ (20 mL) at 0° C., and then extracted with EtOAc (60 mL). The combined organic layers were washed with sat. NaHCO₃ (50 mL), dried over Na₂SO₄, filtered and concentrated under reduced pressure to give the title compound (50 mg) as yellow oil that was used in the next step without further purification.

### Intermediate 34

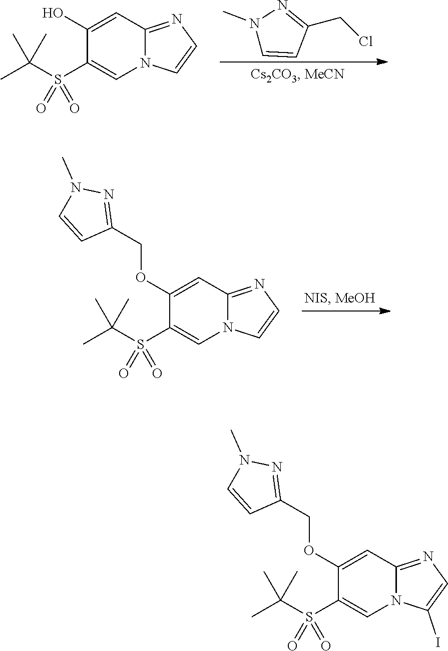

6-(tert-butylsulfonyl)-7-((1-methyl-1H-pyrazol-3-yl)methoxy)imidazo[1,2-a]pyridine: To a solution of 6-(tert-butylsulfonyl)imidazo[1,2-a]pyridin-7-ol (150 mg, 471.87 μmol, 1 eq) and 3-(chloromethyl)-1-methyl-1H-pyrazole (123.23 mg, 943.75 mol, 2 eq) in MeCN (10 mL) was added Cs₂CO₃ (461.24 mg, 1.42 mmol, 3 eq). The mixture was stirred at 80° C. for 4 hours before it was concentrated under reduced pressure to give a residue, which was purified by column chromatography (SiO₂, DCM/MeOH=100/1 to 20/1) to give the title compound (50 mg, 27% yield) as a white solid. ¹H NMR (400 MHz, DMSO-d₆) δ ppm 9.14 (s, 1H), 7.99 (s, 1H), 7.69 (d, J=2.4 Hz, 1H), 7.55 (d, J=1.2 Hz, 1H), 7.30 (s, 1H), 6.35 (d, J=2.4 Hz, 1H), 5.15 (s, 2H), 3.84 (s, 3H), 1.28 (s, 9H).

6-(tert-butylsulfonyl)-3-iodo-7-((1-methyl-1H-pyrazol-3-yl)methoxy)imidazo[1,2-a]pyridine: To a solution of 6-(tert-butylsulfonyl)-7-((1-methyl-1H-pyrazol-3-yl)methoxy)imidazo[1,2-a]pyridine (50 mg, 129.15 μmol, 1 eq) in MeOH (1 mL) was added NIS (43.59 mg, 193.73 μmol, 1.5 eq) at 0° C. The mixture was stirred at 25° C. for 2 hours before it was quenched with Na₂SO₃ (20 mL) and extracted with EtOAc (3×20 mL). The combined organic layers were washed with NaHCO₃ (2×20 mL), dried over Na₂SO₄, filtered and concentrated under reduced pressure to give the title compound (50 mg, 73% yield) as a white solid, which was used into next step without further purification. ¹H NMR (400 MHz, DMSO-d₆) δ ppm 8.51 (s, 1H), 7.73 (s, 1H), 7.70-7.68 (m, 1H), 7.44 (s, 1H), 6.35 (d, J=2.4 Hz, 1H), 5.19 (s, 2H), 3.84 (s, 3H), 1.29 (s, 9H).

### Intermediate 35

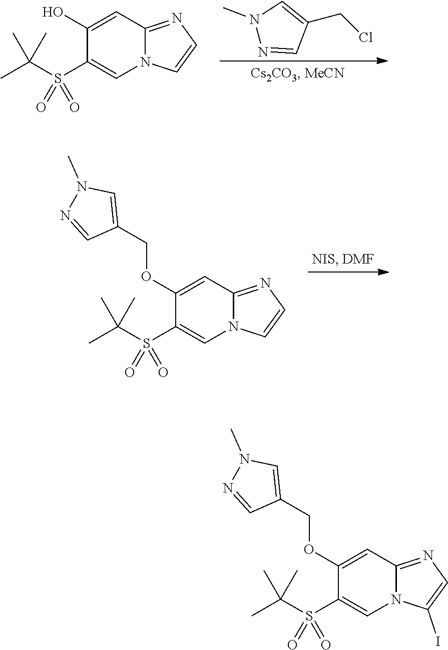

6-(tert-butylsulfonyl)-7-((1-methyl-1H-pyrazol-4-yl)methoxy)imidazo[1,2-a]pyridine: To a solution of 4-(chloromethyl)-1-methyl-1H-pyrazole hydrochloride (88.67 mg, 530.86 μmol, 1 eq) and 6-(tert-butylsulfonyl)imidazo[1,2-a]pyridin-7-ol (150 mg, 530.86 μmol, 1 eq) in MeCN (16 mL) was added Cs₂CO₃ (518.89 mg, 1.59 mmol, 3 eq). The mixture was stirred at 80° C. for 12 hours before it was concentrated under reduced pressure to give a residue, which was purified by column chromatography (SiO₂, DCM/MeOH=100/1 to 10/1) to give the title compound (40 mg, 19% yield) as a yellow solid. [M+H]⁺=349.0. ¹H NMR (400 MHz, DMSO-d₆) δ ppm 9.13 (s, 1H), 7.99 (s, 1H), 7.79 (s, 1H), 7.55 (d, J=1.2 Hz, 1H), 7.50 (s, 1H), 7.26 (s, 1H), 5.10 (s, 2H), 3.84 (s, 3H), 1.27 (s, 9H).

6-(tert-butylsulfonyl)-3-iodo-7-((1-methyl-1H-pyrazol-4-yl)methoxy)imidazo[1,2-a]pyridine: To a solution of 6-(tert-butylsulfonyl)-7-((1-methyl-1H-pyrazol-4-yl)methoxy)imidazo[1,2-a]pyridine (30 mg, 77.49 μmol, 1 eq) in MeOH (4 mL) and H₂O (1 mL) was added NIS (26.15 mg, 116.24 μmol, 1.5 eq). The mixture was stirred at 25° C. for 12 hours before it was quenched with sartd. Na₂SO₃ (20 mL) at 0° C., and then extracted with EtOAc (3×20 mL). The combined organic layers were washed with brine (20 mL), dried over Na₂SO₄, filtered and concentrated under reduced pressure to give the title compound (30 mg, 73% yield) as a white solid, which was used in next step without further purification. [M+H]⁺=474.8. ¹H NMR (400 MHz, DMSO-d₆) δ ppm 8.50 (s, 1H), 7.80 (s, 1H), 7.72 (s, 1H), 7.51 (s, 1H), 7.40 (s, 1H), 5.14 (s, 2H), 3.84 (s, 3H), 1.29 (s, 9H).

### Intermediate 36

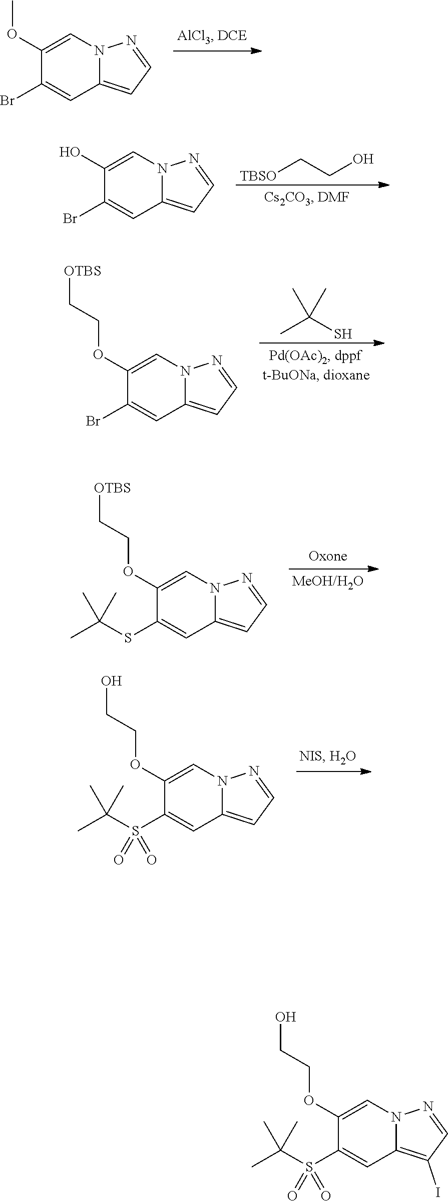

5-bromopyrazolo[1,5-a]pyridin-6-ol: To a solution of 5-bromo-6-methoxypyrazolo[1,5-a]pyridine (500 mg, 1.98 mmol, 1 eq) in DCE (8 mL) was added AlCl₃ (1.32 g, 9.91 mmol, 541.53 μL, 5 eq). The mixture was stirred at 80° C. for 1 hour before it was quenched with saturated Na₂SO₄ (5 mL), neutralized with saturated NaHCO₃, and extracted with DCM (120 mL). The organic phase was dried over Na₂SO₄, filtered and concentrated under reduced pressure to give the title compound (800 mg) as a black-brown solid. [M+H]⁺=214.8. ¹H NMR (400 MHz, DMSO-d₆) δ ppm 10.33 (s, 1H), 8.21 (s, 1H), 8.02 (s, 1H), 7.83 (d, J=1.6 Hz, 1H), 6.47 (d, J=2.0 Hz, 1H).

5-bromo-6-(2-((tert-butyldimethylsilyl)oxy)ethoxy)pyrazolo[1,5-a]pyridine: To a solution of 5-bromopyrazolo[1,5-a]pyridin-6-ol (400 mg, 1.88 mmol, 1 eq) in DMF (5 mL) was added Cs₂CO₃ (1.84 g, 5.63 mmol, 3 eq) and 2-((tert-butyldimethylsilyl)oxy)ethan-1-ol (583.94 mg, 2.44 mmol, 1.3 eq). The mixture was stirred at 80° C. for 12 hours before it was diluted with brine (20 mL), and extracted with EtOAc (140 mL). The combined organic layers were dried over Na₂SO₄, filtered and concentrated under reduced pressure to give a residue, which was purified by column chromatography (SiO₂, petroleum ether/EtOAc=10/1 to 4/1) to give the title compound (80 mg, 10% yield) as a light yellow solid. [M+H]⁺=371.0. ¹H NMR (400 MHz, DMSO-d₆) δ ppm 8.55 (s, 1H), 8.09 (s, 1H), 7.91 (d, J=2.8 Hz, 1H), 6.52 (d, J=2.4 Hz, 1H), 4.16 (t, J=4.8 Hz, 2H), 3.97 (t, J=4.4 Hz, 2H), 0.87 (s, 9H), 0.09 (s, 6H).

6-(2-((tert-butyldimethylsilyl)oxy)ethoxy)-5-(tert-butylthio)pyrazolo[1,5-a]pyridine: A mixture of 5-bromo-6-(2-((tert-butyldimethylsilyl)oxy)ethoxy)pyrazolo[1,5-a]pyridine (80 mg, 193.89 μmol, 1 eq), Pd(OAc)₂ (870.60 g, 3.88 μmol, 0.02 eq), dppf (4.30 mg, 7.76 μmol, 0.04 eq) and t-BuONa (55.90 mg, 581.67 μmol, 3 eq) in dioxane (4 mL) was degassed and purged with N₂ 3 times, and then 2-methylpropane-2-thiol (52.46 mg, 581.67 μmol, 65.49 μL, 3 eq) was added. The mixture was stirred at 90° C. for 5 hours under a N₂ atmosphere before it was filtered and concentrated under reduced pressure to give a residue, which was purified by column chromatography (SiO₂, petroleum ether/EtOAc=20/1 to 5/1) to give the title compound (80 mg, 98% yield) as a yellow solid. [M+H]⁺=381.2. ¹H NMR (400 MHz, DMSO-d₆) δ ppm 8.43 (s, 1H), 7.89 (d, J=2.4 Hz, 1H), 7.86 (s, 1H), 6.59 (d, J=2.4 Hz, 1H), 4.10-4.04 (m, 2H), 3.99-3.93 (m, 2H), 1.29 (s, 9H), 0.88 (s, 9H), 0.11 (s, 6H).

2-((5-(tert-butylsulfonyl)pyrazolo[1,5-a]pyridin-6-yl)oxy)ethan-1-ol: To a solution of 6-(2-((tert-butyldimethylsilyl)oxy)ethoxy)-5-(tert-butylthio)pyrazolo[1,5-a]pyridine (80 mg, 189.17 μmol, 1 eq) in MeOH (3 mL) and H₂O (1 mL) was added Oxone® (174.44 mg, 283.75 μmol, 1.5 eq). The mixture was stirred at 25° C. for 12 hours before it was filtered and concentrated to provide the title compound (57 mg) as a yellow liquid that was used in the next step directly. [M+H]⁺=299.0.

2-((5-(tert-butylsulfonyl)-3-iodopyrazolo[1,5-a]pyridin-6-yl)oxy)ethan-1-ol: To a solution of 2-((5-(tert-butylsulfonyl)pyrazolo[1,5-a]pyridin-6-yl)oxy)ethan-1-ol (57 mg, 191.05 μmol, 1 eq) in H₂O (1 mL) was added NIS (64.47 mg, 286.57 μmol, 1.5 eq). The mixture was stirred at 25° C. for 1 hour before it was quenched with saturated Na₂SO₃ (10 mL) and extracted with EtOAc (30 mL). The combined organic phase was dried over anhydrous sodium sulfate, filtered and concentrated to give a residue, which was purified by column chromatography (SiO₂, petroleum ether/EtOAc=5/1 to 0/1) to give the title compound (90 mg, 99% yield) as a white solid. [M+H]⁺=424.9. ¹H NMR (400 MHz, DMSO-d₆) δ ppm 8.82 (s, 1H), 8.22 (s, 1H), 7.88 (s, 1H), 4.82 (t, J=5.6 Hz, 1H), 4.14 (t, J=5.2 Hz, 2H), 3.75 (q, J=5.2 Hz, 2H), 1.33 (s, 9H).

### Intermediate 37

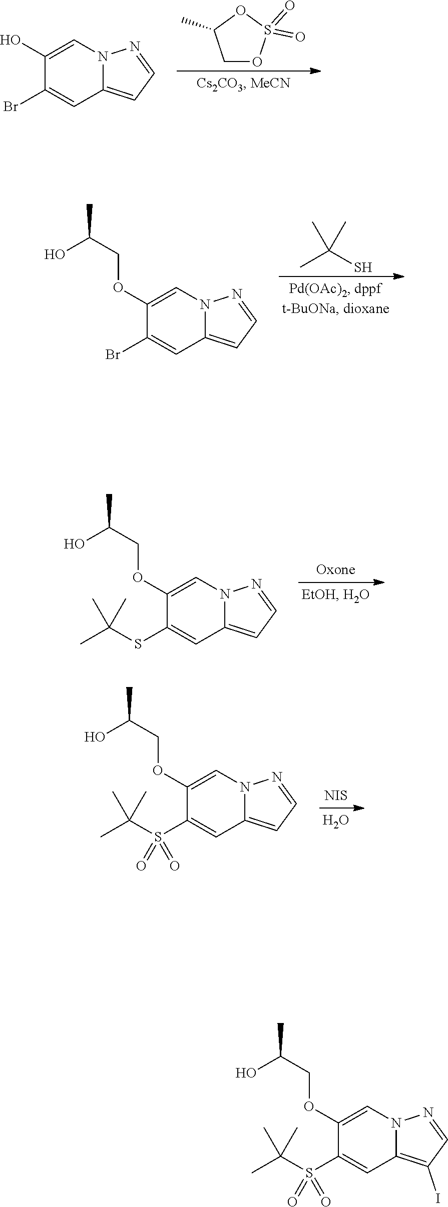

(S)-1-((5-bromopyrazolo[1,5-a]pyridin-6-yl)oxy)propan-2-ol: To a solution of 5-bromopyrazolo[1,5-a]pyridin-6-ol (600 mg, 2.82 mmol, 1 eq) in MeCN (10 mL) was added Cs₂CO₃ (2.75 g, 8.45 mmol, 3 eq) and (S)-4-methyl-1,3,2-dioxathiolane 2,2-dioxide (778.15 mg, 5.63 mmol, 2 eq). The mixture was stirred at 25° C. for 1 hour before it was diluted with H₂O (200 mL) and extracted with EtOAc (3×10 mL). The combined organic layers were washed with brine (10 mL), dried over Na₂SO₄, filtered and concentrated under reduced pressure to give the title compound (40 mg) as a yellow solid. [M+H]⁺=272.9.

(S)-1-((5-(tert-butylthio)pyrazolo[1,5-a]pyridin-6-yl)oxy)propan-2-ol: A mixture of (S)-1-((5-bromopyrazolo[1,5-a]pyridin-6-yl)oxy)propan-2-ol (40 mg, 147.54 μmol, 1 eq), 2-methylpropane-2-thiol (12.67 mg, 140.43 μmol, 15.81 μL, 9.52e-1 eq), Pd(OAc)₂ (3.31 mg, 14.75 μmol, 0.1 eq), dppf (16.36 mg, 29.51 μmol, 0.2 eq) and t-BuONa (42.54 mg, 442.62 μmol, 3 eq) in dioxane (1 mL) was degassed and purged with N₂ 3 times, and then the mixture was stirred at 90° C. for 12 hours under a N₂ atmosphere. The reaction mixture was filtered and concentrated under reduced pressure to give a residue, which was purified by column chromatography (SiO₂, petroleum ether/EtOAc=10/1 to 0/1) to give the title compound (35 mg, 76% yield) as a yellow solid. ¹H NMR (400 MHz, DMSO-d₆) δ ppm 8.42 (s, 1H), 7.89 (d, J=2.4 Hz, 1H), 7.85 (s, 1H), 6.59 (s, 1H), 4.86-4.82 (m, 1H), 4.05-3.98 (m, 1H), 3.94-3.88 (m, 1H), 3.83-3.77 (m, 1H), 1.29 (s, 9H), 1.23-1.19 (m, 3H).

(S)-1-((5-(tert-butylsulfonyl)pyrazolo[1,5-a]pyridin-6-yl)oxy)propan-2-ol: To a solution of (S)-1-((5-(tert-butylthio)pyrazolo[1,5-a]pyridin-6-yl)oxy)propan-2-ol (27.61 mg, 88.63 μmol, 1 eq) in EtOH (0.9 mL) was added Oxone® (81.73 mg, 132.94 μmol, 1.5 eq) and H₂O (0.3 mL). The mixture was stirred at 25° C. for 4 hours before it was filtered and concentrated under reduced pressure to give the title compound (30 mg) as a white solid. [M+H]⁺=313.0.

(S)-1-((5-(tert-butylsulfonyl)-3-iodopyrazolo[1,5-a]pyridin-6-yl)oxy)propan-2-ol: To a solution of (S)-1-((5-(tert-butylsulfonyl)pyrazolo[1,5-a]pyridin-6-yl)oxy)propan-2-ol (30 mg, 96.04 μmol, 1 eq) in H₂O (1 mL) was added NIS (32.41 mg, 144.05 μmol, 1.5 eq). The mixture was stirred at 25° C. for 2 hours before it was diluted with H₂O (20 mL) and extracted with EtOAc (3×10 mL). The combined organic layers were washed with brine (10 mL), dried over Na₂SO₄, filtered and concentrated under reduced pressure to give a residue, which was purified by column chromatography (SiO₂, petroleum ether/EtOAc=10/1 to 0/1) to give the title compound (20 mg, 38% yield) as a white solid. H NMR (400 MHz, DMSO-d₆) δ ppm 8.80 (s, 1H), 8.22 (s, 1H), 7.87 (s, 1H), 4.01-3.90 (m, 3H), 1.33 (s, 9H), 1.21-1.18 (m, 3H).

### Intermediate 38

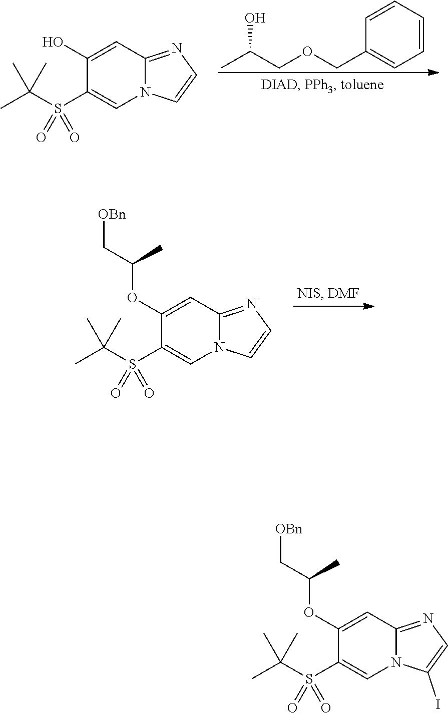

(R)-7-((1-(benzyloxy)propan-2-yl)oxy)-6-(tert-butylsulfonyl)imidazo[1,2-a]pyridine: To a solution of 6-(tert-butylsulfonyl)imidazo[1,2-a]pyridin-7-ol (300 mg, 1.06 mmol, 1 eq) and (S)-1-(benzyloxy)propan-2-ol (176.47 mg, 1.06 mmol, 1 eq) in toluene (10 mL) was added PPh₃ (556.96 mg, 2.12 mmol, 2.0 eq) and DIAD (429.38 mg, 2.12 mmol, 411.68 μL, 2.0 eq). The mixture was stirred at 100° C. for 12 hours under a N₂ atmosphere. The reaction mixture was concentrated under reduced pressure to give a residue, which was purified by column chromatography (SiO₂, petroleum ether/EtOAc=1/0 to 3/1) to give the title compound (100 mg, 21% yield) as yellow oil. ¹H NMR (400 MHz, DMSO-d₆) δ ppm 9.13 (s, 1H), 7.98 (s, 1H), 7.66-7.63 (m, 1H), 7.36-7.21 (m, 6H), 4.98-4.91 (m, 1H), 4.60-4.49 (m, 2H), 3.67-3.58 (m, 2H), 1.33-1.26 (m, 12H).

(R)-7-((1-(benzyloxy)propan-2-yl)oxy)-6-(tert-butylsulfonyl)-3-iodoimidazo[1,2-a]pyridine: To a solution of (R)-7-((1-(benzyloxy)propan-2-yl)oxy)-6-(tert-butylsulfonyl)imidazo[1,2-a]pyridine (100 mg, 223.60 μmol, 1 eq) in DMF (3 mL) was added NIS (60.37 mg, 268.32 μmol, 1.2 eq) at 0° C. The mixture was stirred at 25° C. for 1 hour under a N₂ atmosphere before it was concentrated under reduced pressure to give a residue, which was purified by column chromatography (SiO₂, petroleum ether/EtOAc=1/0 to 1/1) to give the title compound (80 mg, 61% yield) as yellow oil. ¹H NMR (400 MHz, DMSO-d₆) δ ppm 8.51 (s, 1H), 7.71 (s, 1H), 7.38 (s, 1H), 7.33-7.22 (m, 5H), 5.04-4.95 (m, 1H), 4.60-4.47 (m, 2H), 3.65 (d, J=4.8 Hz, 2H), 1.35-1.31 (m, 9H), 1.29 (d, J=6.0 Hz, 3H).

### Intermediate 39

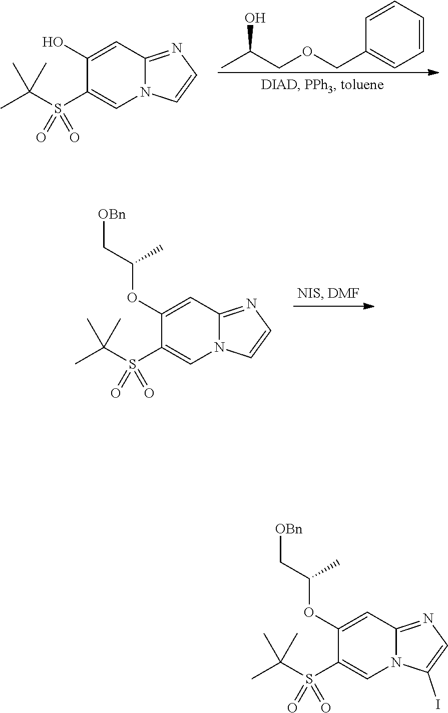

(S)-7-((1-(benzyloxy)propan-2-yl)oxy)-6-(tert-butylsulfonyl)imidazo[1,2-a]pyridine: To a solution of 6-(tert-butylsulfonyl)imidazo[1,2-a]pyridin-7-ol (300 mg, 1.06 mmol, 1 eq) in toluene (9 mL) was added (R)-1-(benzyloxy)propan-2-ol (529.42 mg, 3.19 mmol, 3 eq), DIAD (429.38 mg, 2.12 mmol, 411.67 μL, 2 eq) and PPh₃ (556.95 mg, 2.12 mmol, 2 eq). The mixture was stirred at 100° C. for 12 hours before it was concentrated under reduced pressure to give a residue, which was purified by column chromatography (SiO₂, petroleum ether/EtOAc=100/1 to 0/1) to give the title compound (120 mg, 22% yield) as a yellow solid. ¹H NMR (400 MHz, DMSO-d₆) δ ppm 9.14 (s, 1H), 7.99 (s, 1H), 7.39-7.19 (m, 7H), 5.01-4.90 (m, 1H), 4.60-4.52 (m, 2H), 3.70-3.59 (m, 2H), 1.32 (s, 9H), 1.29 (d, J=6.4 Hz, 3H).

(S)-7-((1-(benzyloxy)propan-2-yl)oxy)-6-(tert-butylsulfonyl)-3-iodoimidazo[1,2-a]pyridine: To a solution of (S)-7-((1-(benzyloxy)propan-2-yl)oxy)-6-(tert-butylsulfonyl)imidazo[1,2-a]pyridine (120 mg, 238.51 μmol, 1 eq) in DMF (2 mL) was added NIS (64.39 mg, 286.21 μmol, 1.2 eq) at 0° C. The mixture was stirred at 25° C. for 1 hour before it was diluted with H₂O (20 mL) and extracted with EtOAc (3×30 mL). The combined organic layers were washed with brine (30 mL), dried over Na₂SO₄, filtered and concentrated under reduced pressure to give a residue, which was purified by column chromatography (SiO₂, petroleum ether/EtOAc=100/0 to 0/1) to give the title compound (60 mg, 43% yield) as a yellow solid. ¹H NMR (400 MHz, DMSO-d₆) δ ppm 8.51 (s, 1H), 7.72 (s, 1H), 7.38 (s, 1H), 7.34-7.21 (m, 5H), 5.05-4.94 (m, 1H), 4.63-4.46 (m, 2H), 3.65 (d, J=4.8 Hz, 2H), 1.33 (s, 9H), 1.29 (d, J=6.0 Hz, 3H).

### Intermediate 40

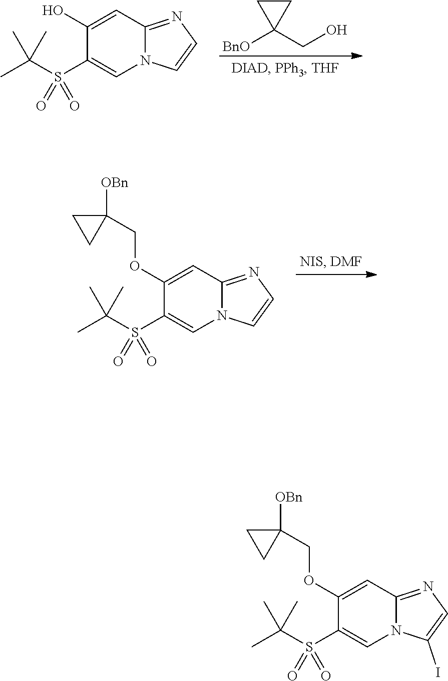 [SMILES: CC(C)(C)S(=O)(=O)c1cn2ccnc2cc1O ; OCC1(OCc2ccccc2)CC1 ; CC(C)(C)S(=O)(=O)c1cn2ccnc2cc1OCC1(OCc2ccccc2)CC1 ; CC(C)(C)S(=O)(=O)c1cn2c(I)cnc2cc1OCC1(OCc2ccccc2)CC1]

7-((1-(benzyloxy)cyclopropyl)methoxy)-6-(tert-butylsulfonyl)imidazo[1,2-a]pyridine: To a solution of (1-(benzyloxy)cyclopropyl)methanol (250 mg, 884.76 μmol, 1 eq) in THF (12.5 mL) was added 6-(tert-butylsulfonyl)imidazo[1,2-a]pyridin-7-ol (262.82 mg, 1.33 mmol, 1.5 eq) and PPh₃ (464.12 mg, 1.77 mmol, 2 eq). The mixture was purged with N₂ 3 times before DIAD (447.27 mg, 2.21 mmol, 428.83 μL, 2.5 eq) was slowly added. The resulting mixture was stirred at 25° C. for 4 hours under a N₂ atmosphere, and then it was concentrated to give a residue, which was purified by RP-HPLC to give the title compound (180 mg, 44% yield) as a light yellow solid. ¹H NMR (400 MHz, DMSO-d₆) δ ppm 9.15 (s, 1H), 8.00 (s, 1H), 7.55 (s, 1H), 7.32-7.23 (m, 5H), 7.15 (s, 1H), 4.72 (s, 2H), 4.35 (s, 2H), 1.35 (s, 9H), 1.01-0.95 (m, 2H), 0.84-0.77 (m, 2H).

7-((1-(benzyloxy)cyclopropyl)methoxy)-6-(tert-butylsulfonyl)-3-iodoimidazo[1,2-a]pyridine: To a solution of 7-((1-(benzyloxy)cyclopropyl)methoxy)-6-(tert-butylsulfonyl)imidazo[1,2-a]pyridine (180 mg, 390.82 μmol, 1 eq) in DMF (3 mL) was added NIS (105.51 mg, 468.98 μmol, 1.2 eq). The reaction mixture was diluted with water (20 mL) and extracted with DCM (3×10 mL), and then the combined organic layers were washed with sat. Na₂SO₃ (2×15 mL) and sat. NaHCO₃ (2×10 mL). The combined organic layers were extracted with EtOAc (10 mL×2), dried over Na₂SO₄, filtered and concentrated under reduced pressure to give the title compound (135 mg) as a yellow solid. ¹H NMR (400 MHz, DMSO-d₆) δ ppm 8.52 (s, 1H), 7.72 (s, 1H), 7.32-7.21 (m, 6H), 4.71 (s, 2H), 4.38 (s, 2H), 1.36 (s, 9H), 1.03-0.96 (m, 2H), 0.83-0.77 (m, 2H).

### Intermediate 41

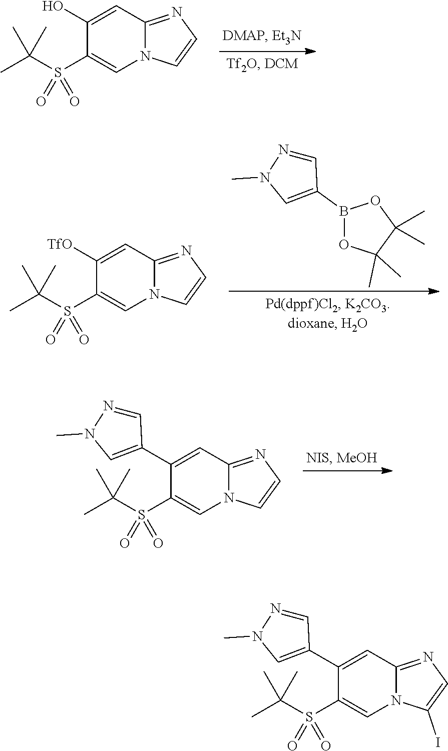

6-(tert-butylsulfonyl)imidazo[1,2-a]pyridin-7-yl trifluoromethanesulfonate: To a solution of 6-(tert-butylsulfonyl)imidazo[1,2-a]pyridin-7-ol (200 mg, 707.81 μmol, 1 eq) in DCM (5 mL) was added DMAP (86.47 mg, 707.81 μmol, 1 eq) and Et₃N (214.87 mg, 2.12 mmol, 295.55 μL, 3 eq) at 0° C. After addition, the mixture was stirred at this temperature for 0.25 hours, and then Tf₂O (399.40 mg, 1.42 mmol, 233.57 μL, 2 eq) was added dropwise at 0° C. The mixture was stirred at 20° C. for 12 hours before it was filtered and concentrated under reduced pressure to give a residue, which was purified by column chromatography (SiO₂, 0-6% MeOH/DCM) to give the title compound (216 mg, 71% yield) as a white solid. ¹H NMR (400 MHz, DMSO-d₆) δ ppm 9.47 (s, 1H), 8.31 (s, 1H), 7.89 (s, 1H), 7.85 (s, 1H), 1.33 (s, 9H).

6-(tert-butylsulfonyl)-7-(1-methyl-1H-pyrazol-4-yl)imidazo[1,2-a]pyridine: A mixture of 6-(tert-butylsulfonyl)imidazo[1,2-a]pyridin-7-yl trifluoromethanesulfonate (120 mg, 279.53 μmol, 1 eq), 1-methyl-4-(4,4,5,5-tetramethyl-1,3,2-dioxaborolan-2-yl)-1H-pyrazole (69.79 mg, 335.43 μmol, 1.2 eq), Pd(dppf)Cl₂ (20.45 mg, 27.95 μmol, 0.1 eq) and K₂CO₃ (115.90 mg, 838.58 μmol, 3 eq) in 1,4-dioxane (3 mL) and H₂O (0.75 mL) was degassed and purged with N₂ 3 times, and then the mixture was stirred at 80° C. for 12 hours under a N₂ atmosphere. The reaction mixture was quenched with NH₄Cl (10 mL) at 0° C., and then diluted with EtOAc (10 mL) and extracted with EtOAc (3×10 mL). The combined organic layers were washed with brine (3×10 mL), dried over Na₂SO₄, filtered and concentrated under reduced pressure to give a residue, which was purified by column chromatography (SiO₂, 0-6% EtOAc/petroleum ether) to give the title compound (65 mg, 66% yield) as a white solid. ¹H NMR (400 MHz, DMSO-d₆) δ ppm 9.38 (s, 1H), 8.22 (s, 1H), 7.95 (s, 1H), 7.75 (s, 1H), 7.65 (s, 1H), 7.52 (s, 1H), 3.88 (s, 3H), 1.08 (s, 9H).

6-(tert-butylsulfonyl)-3-iodo-7-(1-methyl-1H-pyrazol-4-yl)imidazo[1,2-a]pyridine: To a solution of 6-(tert-butylsulfonyl)-7-(1-methyl-1H-pyrazol-4-yl)imidazo[1,2-a]pyridine (60 mg, 169.60 μmol, 1 eq) in MeOH (2 mL) was added NIS (76.32 mg, 339.20 μmol, 2 eq). The mixture was stirred at 80° C. for 12 hours before it was quenched with Na₂SO₃ (10 mL) at 0° C., and then diluted with H₂O (10 mL) and extracted with EtOAc (3×10 mL). The combined organic layers were washed with brine (3×10 mL), dried over Na₂SO₄, filtered and concentrated under reduced pressure to give a residue, which was purified by column chromatography (SiO₂, 0-8% EtOAc/petroleum ether) to give the title compound (30 mg, 36% yield) as a white solid. ¹H NMR (400 MHz, DMSO-d₆) δ ppm 8.79 (s, 1H), 7.99 (s, 1H), 7.94 (s, 1H), 7.69 (s, 1H), 7.63 (s, 1H), 3.89 (s, 3H), 1.10 (s, 9H).

### Intermediate 42

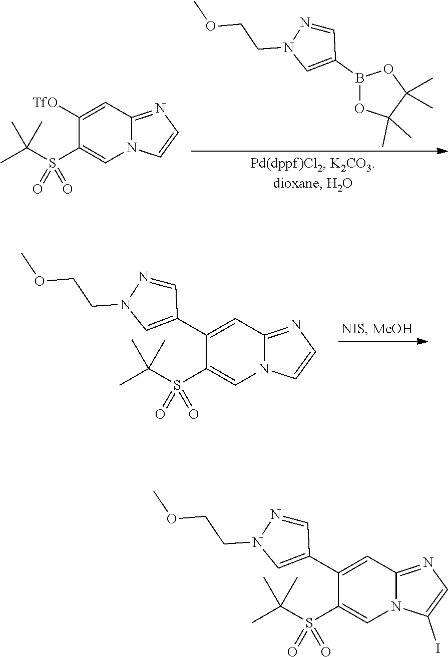

6-(tert-butylsulfonyl)-7-(1-(2-methoxyethyl)-1H-pyrazol-4-yl)imidazo[1,2-a]pyridine: A mixture of 6-(tert-butylsulfonyl)imidazo[1,2-a]pyridin-7-yl trifluoromethanesulfonate (150 mg, 310.59 μmol, 1 eq), 1-(2-methoxyethyl)-4-(4,4,5,5-tetramethyl-1,3,2-dioxaborolan-2-yl)-1H-pyrazole (234.91 mg, 931.76 μmol, 3 eq), K₂CO₃ (128.77 mg, 931.76 μmol, 3 eq) and Pd(dppf)Cl₂ (45.45 mg, 62.12 μmol, 0.2 eq) in 1,4-dioxane (3 mL) and H₂O (0.3 mL) was degassed and purged with N₂ 3 times, and then the mixture was stirred at 80° C. for 4 hours under a N₂ atmosphere. The reaction mixture was filtered and concentrated under reduced pressure to give a residue, which was purified by column chromatography (SiO₂, DCM/MeOH=1/0 to 10/1) to give the title compound (100 mg, 80% yield) as a yellow solid. [M+H]⁺=363.0. ¹H NMR (400 MHz, DMSO-d₆) δ ppm 9.38 (s, 1H), 8.22 (s, 1H), 8.00 (s, 1H), 7.75 (s, 1H), 7.68 (s, 1H), 7.54 (s, 1H), 4.29 (t, J=5.2 Hz, 2H), 3.69 (t, J=5.2 Hz, 2H), 3.24 (s, 3H), 1.07 (s, 9H).

6-(tert-butylsulfonyl)-3-iodo-7-(1-(2-methoxyethyl)-1H-pyrazol-4-yl)imidazo[1,2-a]pyridine: To a solution of 6-(tert-butylsulfonyl)-7-(1-(2-methoxyethyl)-1H-pyrazol-4-yl)imidazo[1,2-a]pyridine (90 mg, 223.48 μmol, 1 eq) in MeOH (2 mL) was added NIS (150.84 mg, 670.45 μmol, 3 eq). The mixture was stirred at 0° C. for 2 hours. The reaction mixture was filtered and concentrated under reduced pressure to give a residue that was purified by column chromatography (SiO₂, DCM/MeOH=1/0 to 10/1) to give the title compound (90 mg, 74% yield) as a white solid. [M+H]⁺=489.0. ¹H NMR (400 MHz, DMSO-d₆) δ ppm 8.79 (s, 1H), 8.04 (s, 1H), 7.93 (s, 1H), 7.72 (s, 1H), 7.68-7.61 (m, 1H), 4.30 (t, J=5.2 Hz, 2H), 3.69 (t, J=5.2 Hz, 2H), 3.24 (s, 3H), 1.07 (s, 9H).

### Intermediate 43

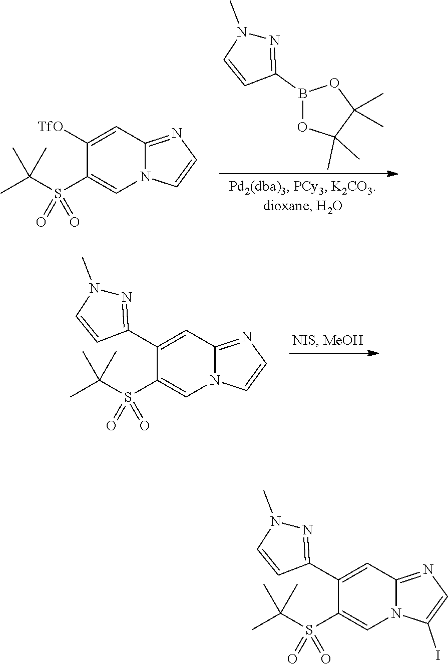

6-(tert-butylsulfonyl)-7-(1-methyl-1H-pyrazol-3-yl)imidazo[1,2-a]pyridine: A mixture of 6-(tert-butylsulfonyl)imidazo[1,2-a]pyridin-7-yl trifluoromethanesulfonate (100 mg, 232.94 μmol, 1 eq), 1-methyl-3-(4,4,5,5-tetramethyl-1,3,2-dioxaborolan-2-yl)-1H-pyrazole (96.93 mg, 465.88 μmol, 2 eq), K₂CO₃ (96.58 mg, 698.82 μmol, 3 eq), Pd₂(dba)₃ (21.33 mg, 23.29 μmol, 0.1 eq) and PCy₃ (13.06 mg, 46.59 μmol, 15.10 μL, 0.2 eq) in dioxane (3 mL) and H₂O (0.75 mL) was degassed and purged with N₂ 3 times, and then the mixture was stirred at 80° C. for 12 hours under a N₂ atmosphere. The reaction mixture was quenched with NH₄Cl (10 mL) at 0° C., and then diluted with H₂O (10 mL) and extracted with EtOAc (3×10 mL). The combined organic layers were washed with brine (10 mL×3), dried over Na₂SO₄, filtered and concentrated under reduced pressure to give a residue, which was purified by column chromatography (SiO₂, 0-6% EtOAc/petroleum ether) to give the title compound (260 mg, 73% yield) as a white solid. ¹H NMR (400 MHz, DMSO-d₆) δ ppm 9.38 (s, 1H), 8.25 (s, 1H), 7.78 (s, 1H), 7.69 (d, J=2.0 Hz, 1H), 7.56 (s, 1H), 6.56 (d, J=2.0 Hz, 1H), 3.88 (s, 3H), 1.10 (s, 9H).

6-(tert-butylsulfonyl)-3-iodo-7-(1-methyl-1H-pyrazol-3-yl)imidazo[1,2-a]pyridine: To a solution of 6-(tert-butylsulfonyl)-7-(1-methyl-1H-pyrazol-3-yl)imidazo[1,2-a]pyridine (50 mg, 141.33 μmol, 1 eq) in MeOH (2 mL) and H₂O (0.5 mL) was added NIS (38.16 mg, 169.60 μmol, 1.2 eq). The mixture was stirred at 20° C. for 2 hours. The reaction mixture was quenched with NH₄Cl (10 mL) at 0° C., and then diluted with H₂O (10 mL) and extracted with EtOAc (3×10 mL). The combined organic layers were washed with brine (3×10 mL), dried over Na₂SO₄, filtered and concentrated under reduced pressure to give a residue, which was purified by column chromatography (SiO₂, 0-6% EtOAc/petroleum ether) to give the title compound (43 mg, 62% yield) as a white solid. ¹H NMR (400 MHz, DMSO-d₆) δ ppm 8.78 (s, 1H), 7.97 (s, 1H), 7.72 (d, J=2.4 Hz, 1H), 7.66 (s, 1H), 6.61 (d, J=2.0 Hz, 1H), 3.89 (s, 3H), 1.11 (s, 9H).

### Intermediate 44

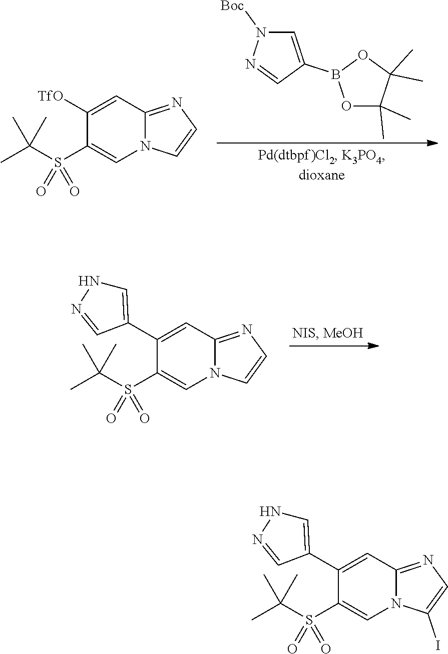 [SMILES: CC(C)(C)S(=O)(=O)c1cn2ccnc2cc1OS(=O)(=O)C(F)(F)F ; CC(C)(C)OC(=O)n1cc(B2OC(C)(C)C(C)(C)O2)cn1 ; CC(C)(C)S(=O)(=O)c1cn2ccnc2cc1-c1cn[nH]c1 ; CC(C)(C)S(=O)(=O)c1cn2c(I)cnc2cc1-c1cn[nH]c1]

6-(tert-butylsulfonyl)-7-(1H-pyrazol-4-yl)imidazo[1,2-a]pyridine: A mixture of 6-(tert-butylsulfonyl)imidazo[1,2-a]pyridin-7-yl trifluoromethanesulfonate (200 mg, 465.88 μmol, 1 eq), tert-butyl 4-(4,4,5,5-tetramethyl-1,3,2-dioxaborolan-2-yl)-1H-pyrazole-1-carboxylate (274.08 mg, 931.76 μmol, 2 eq), K₃PO₄ (296.67 mg, 1.40 mmol, 3 eq) and Pd(dtbpf)Cl₂ (30.36 mg, 46.59 μmol, 0.1 eq) in dioxane (2.4 mL) and H₂O (0.6 mL) was degassed and purged with N₂, and then the mixture was stirred at 80° C. for 4 hours under a N₂ atmosphere. The reaction mixture was concentrated under reduced pressure to give a residue, which was purified by column chromatography (SiO₂, DCM/MeOH=99/1 to 90/10) to give the title compound (100 mg, 56% yield) as a yellow solid. [M+H]⁺=305.2. ¹H NMR (400 MHz, DMSO-d₆) δ ppm 13.06-12.86 (m, 1H), 9.39 (s, 1H), 8.22 (s, 1H), 7.98 (s, 1H), 7.75 (s, 1H), 7.72 (s, 1H), 7.54 (s, 1H), 1.05 (s, 9H).

6-(tert-butylsulfonyl)-3-iodo-7-(1H-pyrazol-4-yl)imidazo[1,2-a]pyridine: To a mixture of 6-(tert-butylsulfonyl)-7-(1H-pyrazol-4-yl)imidazo[1,2-a]pyridine (50 mg, 131.42 μmol, 1 eq) in MeOH (1.2 mL) and H₂O (0.4 mL) was added NIS (44.35 mg, 197.13 μmol, 1.5 eq) at 0° C., and then the mixture was stirred at 20° C. for 1 hour before it was diluted with saturated NaHCO₃ (10 mL) and extracted with DCM (3×20 mL). The combined organic extracts were dried over Na₂SO₄, filtered and concentrated under reduced pressure to give a residue, which was purified by column chromatography (SiO₂, petroleum ether/EtOAc=1/0 to 0/1) to give the title compound (35 mg, 56% yield) as a yellow solid. ¹H NMR (400 MHz, CD₃OD) δ ppm 8.98 (s, 1H), 8.05 (s, 1H), 7.87 (s, 1H), 7.84 (s, 1H), 7.62 (s, 1H), 1.15 (s, 9H).

### Intermediate 45

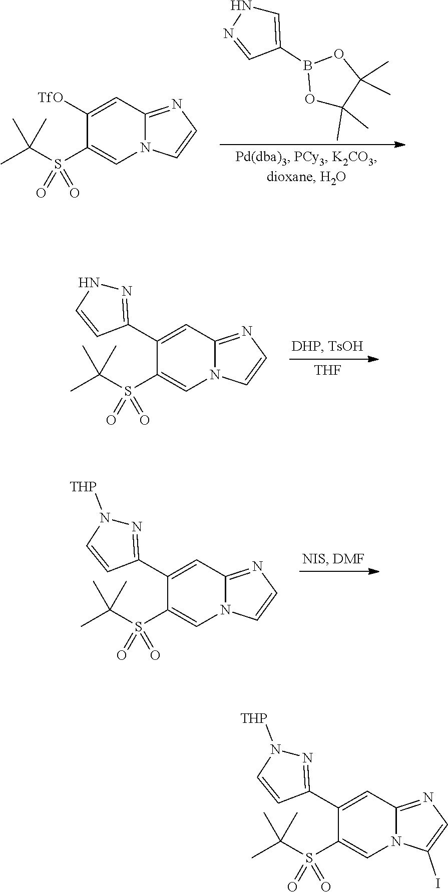

6-(tert-butylsulfonyl)-7-(1H-pyrazol-3-yl)imidazo[1,2-a]pyridine: A mixture of 6-(tert-butylsulfonyl)imidazo[1,2-a]pyridin-7-yl trifluoromethanesulfonate (400 mg, 931.76 μmol, 1 eq), 3-(4,4,5,5-tetramethyl-1,3,2-dioxaborolan-2-yl)-1H-pyrazole (401.77 mg, 1.86 mmol, 2 eq), Pd₂(dba)₃ (85.32 mg, 93.18 μmol, 0.1 eq), PCy₃ (261.29 mg, 931.76 μmol, 302.07 μL, 1 eq) and K₂CO₃ (386.32 mg, 2.80 mmol, 3 eq) in 1,4-dioxane (4 mL) and H₂O (1 mL) was degassed and purged with N₂ 3 times, and then the mixture was stirred at 100° C. for 2 hours under a N₂ atmosphere. The reaction mixture was concentrated under reduced pressure and the residue was purified by RP-HPLC to give the title compound (80 mg, 28% yield) as a yellow solid. ¹H NMR (400 MHz, DMSO-d₆) δ ppm 13.03-12.92 (m, 1H), 9.39 (s, 1H), 8.26 (s, 1H), 7.78 (s, 1H), 7.75 (s, 1H), 7.73 (s, 1H), 7.57 (s, 1H), 6.61 (s, 1H), 1.08 (s, 9H).

6-(tert-butylsulfonyl)-7-(1-(tetrahydro-2H-pyran-2-yl)-1H-pyrazol-3-yl)imidazo[1,2-a]pyridine: To a solution of 6-(tert-butylsulfonyl)-7-(1H-pyrazol-3-yl)imidazo[1,2-a]pyridine (130 mg, 427.12 μmol, 1 eq) in THF (10 mL) was added TsOH (110.33 mg, 640.67 μmol, 1.5 eq) and DHP (179.64 mg, 2.14 mmol, 195.26 μL, 5 eq). The mixture was stirred at 80° C. for 2 hours before it was concentrated under reduced pressure to give a residue that was purified by column chromatography (SiO₂, DCM/MeOH=100/0 to 95/5) to give the title compound (70 mg, 34% yield) as a white solid. ¹H NMR (400 MHz, CDCl₃) δ ppm 9.00 (s, 1H), 7.86 (s, 1H), 7.81 (d, J=1.2 Hz, 1H), 7.74 (s, 1H), 7.62 (d, J=2.4 Hz, 1H), 6.82 (d, J=2.4 Hz, 1H), 5.43 (dd, J=8.8, 2.8 Hz, 1H), 4.10-4.04 (m, 1H), 3.75-3.71 (m, 1H), 2.10-2.05 (m, 2H), 1.64-1.62 (m, 2H), 1.56-1.53 (m, 2H), 1.18 (s, 9H).

6-(tert-butylsulfonyl)-3-iodo-7-(1-(tetrahydro-2H-pyran-2-yl)-1H-pyrazol-3-yl)imidazo[1,2-a]pyridine: To a solution of 6-(tert-butylsulfonyl)-7-(1-(tetrahydro-2H-pyran-2-yl)-1H-pyrazol-3-yl)imidazo[1,2-a]pyridine (70 mg, 144.15 μmol, 1 eq) in DMF (1 mL) was added NIS (162.16 mg, 720.75 μmol, 5 eq) at 0° C. The mixture was stirred at 0° C. for 1 hour before it was diluted with H₂O (30 mL) and extracted with EtOAc (3×30 mL). The combined organic layers were washed with brine (30 mL), dried over Na₂SO₄, filtered and concentrated under reduced pressure to give a residue, which was purified by column chromatography (SiO₂, DCM/MeOH=100/0 to 9/1) to give the title compound (60 mg, 73% yield) as a white solid. ¹H NMR (400 MHz, CDCl₃) δ ppm 8.95 (s, 1H), 7.92 (s, 1H), 7.87 (s, 1H), 7.63 (d, J=2.4 Hz, 1H), 6.84 (d, J=2.4 Hz, 1H), 5.44 (dd, J=8.8, 2.8 Hz, 1H), 4.14-4.03 (m, 1H), 3.76-3.71 (m, 1H), 2.13-2.07 (m, 2H), 1.72-1.69 (m, 2H), 1.65-1.61 (m, 2H), 1.19 (s, 9H).

### Intermediate 46

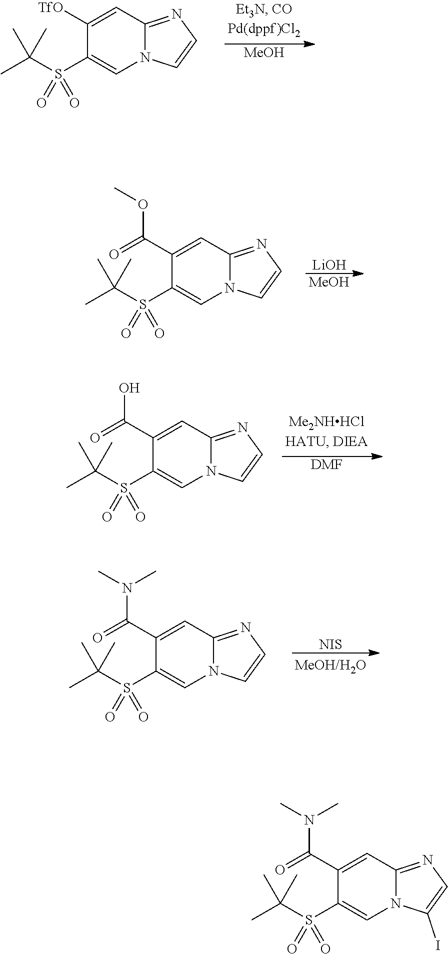

methyl 6-(tert-butylsulfonyl)imidazo[1,2-a]pyridine-7-carboxylate: To a solution of 6-(tert-butylsulfonyl)imidazo[1,2-a]pyridin-7-yl trifluoromethanesulfonate (510 mg, 1.19 mmol, 1 eq) in MeOH (15 mL) was added Pd(dppf)Cl₂ (86.93 mg, 118.80 μmol, 0.1 eq) and Et₃N (360.63 mg, 3.56 mmol, 496.06 μL, 3 eq) under a N₂ atmosphere. The suspension was degassed and purged with CO 3 times. The mixture was stirred under CO (40 Psi) at 80° C. for 12 hours before it was concentrated under reduced pressure to give a residue, which was purified by column chromatography (SiO₂, petroleum ether/EtOAc=1/0 to 0/1) to give the title compound (220 mg, 56% yield) as a brown solid. ¹H NMR (400 MHz, DMSO-d₆) δ ppm 9.36 (s, 1H), 8.26 (s, 1H), 7.89 (s, 1H), 7.85 (s, 1H), 3.81 (s, 9H), 1.35 (s, 9H).

6-(tert-butylsulfonyl)imidazo[1,2-a]pyridine-7-carboxylic acid: To a solution of methyl 6-(tert-butylsulfonyl)imidazo[1,2-a]pyridine-7-carboxylate (220 mg, 668.15 μmol, 1 eq) in MeOH (4 mL) and H₂O (1 mL) was added LiOH·H₂O (280.38 mg, 6.68 mmol, 10 eq). The mixture was stirred at 50° C. for 12 hours before it was concentrated under reduced pressure to give a residue, which was purified by RP-HPLC to yield the title compound (170 mg, 81% yield) as a light yellow solid. ¹H NMR (400 MHz, DMSO-d₆) δ ppm 9.07 (s, 1H), 8.07 (s, 1H), 7.62 (d, J=1.2 Hz, 1H), 7.12 (s, 1H), 1.33 (s, 9H).

6-(tert-butylsulfonyl)-N,N-dimethylimidazo[1,2-a]pyridine-7-carboxamide: To a solution of 6-(tert-butylsulfonyl)imidazo[1,2-a]pyridine-7-carboxylic acid (80 mg, 255.03 μmol, 1 eq) in DMF (2 mL) was added HATU (116.37 mg, 306.04 μmol, 1.2 eq), DIEA (98.88 mg, 765.10 μmol, 133.27 μL, 3 eq) and Me₂NH·HCl (24.96 mg, 306.04 μmol, 1.2 eq). The mixture was stirred at 25° C. for 2 hours before it was quenched with H₂O (1 mL). The mixture was purified by RP-HPLC to yield the title compound (55 mg, 63% yield) as a white solid. ¹H NMR (400 MHz, DMSO-d₆) δ ppm 9.34 (s, 1H), 8.23 (s, 1H), 7.80 (s, 1H), 7.55 (s, 1H), 2.94 (s, 3H), 2.70 (s, 3H), 1.31 (m, 9H).

6-(tert-butylsulfonyl)-3-iodo-N,N-dimethylimidazo[1,2-a]pyridine-7-carboxamide: To a solution of 6-(tert-butylsulfonyl)-N,N-dimethylimidazo[1,2-a]pyridine-7-carboxamide (50 mg, 145.45 μmol, 1 eq) in MeOH (2 mL) and H₂O (2 mL) was added NIS (654.48 mg, 2.91 mmol, 20 eq). The mixture was stirred at 25° C. for 2 hours before it was diluted with H₂O (10 mL) and extracted with EtOAc (3×20 mL). The combined organic layers were washed with brine (20 mL), dried over Na₂SO₄, filtered and concentrated under reduced pressure to give a residue, which was purified by column chromatography (SiO₂, petroleum ether/EtOAc=1/0 to 0/1) to yield the title compound (60 mg, 85% yield) as a white solid. ¹H NMR (400 MHz, DMSO-d₆) δ ppm 8.55 (s, 1H), 7.98 (s, 1H), 7.66 (s, 1H), 2.95 (s, 3H), 2.69 (s, 3H), 1.33 (s, 9H).

### Intermediate 47

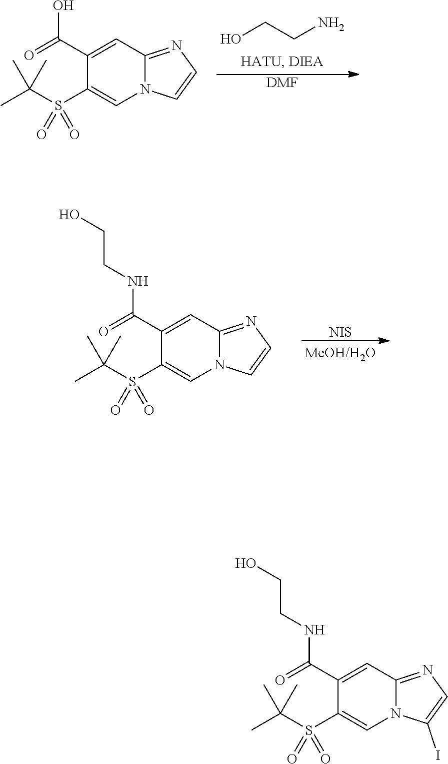

6-(tert-butylsulfonyl)-N-(2-hydroxyethyl)imidazo[1,2-a]pyridine-7-carboxamide: To a solution of 6-(tert-butylsulfonyl)imidazo[1,2-a]pyridine-7-carboxylic acid (70 mg, 223.15 μmol, 1 eq) and 2-aminoethan-1-ol (16.36 mg, 267.79 μmol, 16.16 μL, 1.2 eq) in DMF (2 mL) was added HATU (169.70 mg, 446.31 μmol, 2 eq) and DIEA (43.26 mg, 334.73 μmol, 58.30 μL, 1.5 eq). The mixture was stirred at 20° C. for 1 hour before it was purified by RP-HPLC to give the title compound (17 mg, 21% yield) as a white solid. [M+H]⁺=326.0. ¹H NMR (400 MHz, DMSO-d₆) δ ppm 9.29 (s, 1H), 8.33-8.25 (m, 1H), 8.23 (s, 1H), 7.80 (s, 1H), 7.61 (s, 1H), 4.70-4.54 (m, 1H), 3.54-3.46 (m, 2H), 3.26 (d, J=6.0 Hz, 2H), 1.34 (s, 9H).

6-(tert-butylsulfonyl)-N-(2-hydroxyethyl)-3-iodoimidazo[1,2-a]pyridine-7-carboxamide: To a solution of 6-(tert-butylsulfonyl)-N-(2-hydroxyethyl)imidazo[1,2-a]pyridine-7-carboxamide (15 mg, 41.49 μmol, 1 eq) in MeOH (1.2 mL) and H₂O (0.4 mL) was added NIS (93.35 mg, 414.90 μmol, 10 eq) at 0° C. The mixture was stirred at 0° C. for 1 hour before it was diluted with saturated NaHCO₃ (3 mL) and extracted with EtOAc (3×10 mL). The combined organic extracts were dried over Na₂SO₄, filtered and concentrated under reduced pressure to give the title compound (30 mg) as a yellow solid, which was used in the next step without purification. ¹H NMR (400 MHz, DMSO-d₆) δ ppm 8.53 (s, 1H), 8.33 (t, J=5.2 Hz, 1H), 8.00 (s, 1H), 7.73 (s, 1H), 4.66 (t, J=6.0 Hz, 1H), 3.55-3.46 (m, 2H), 3.31-3.21 (m, 2H), 1.35 (s, 9H).

### Intermediate 48

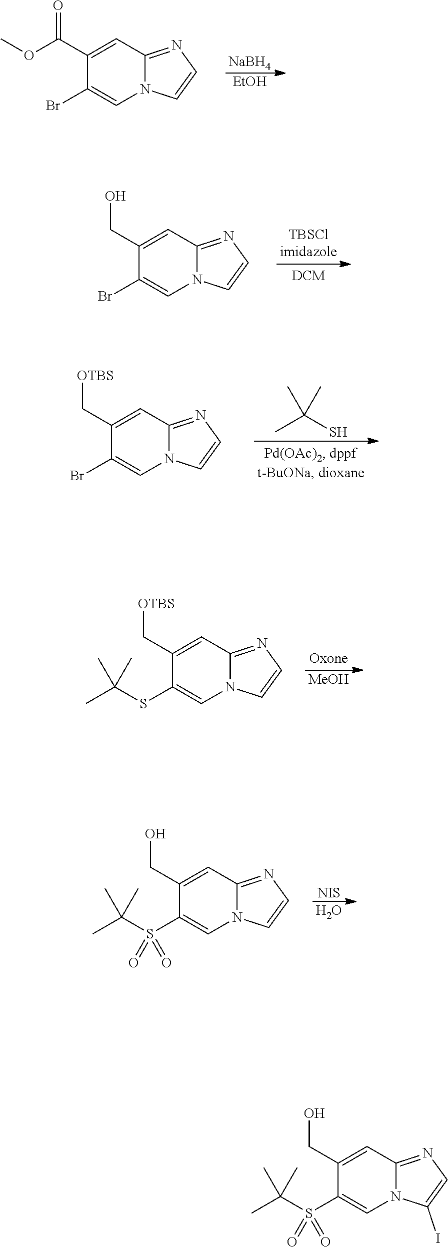

(6-bromoimidazo[1,2-a]pyridin-7-yl)methanol: A mixture of methyl 6-bromoimidazo[1,2-a]pyridine-7-carboxylate (1 g, 3.53 mmol, 1 eq) in EtOH (10 mL) was degassed and purged with N₂ 3 times, and then NaBH₄ (400.47 mg, 10.59 mmol, 3 eq) was added to the mixture at 0° C. The mixture was stirred at 20° C. for 2 hours under a N₂ atmosphere before it was quenched with 1N HCl (1 mL) at 0° C., and then diluted with H₂O (15 mL) and extracted with EtOAc (3×15 mL). The combined organic layers were dried over Na₂SO₄, filtered and concentrated under reduced pressure to give the title compound (590 mg, 66% yield) as a white solid, which was used for the next step without purification. [M+H]⁺=226.9. ¹H NMR (400 MHz, DMSO-d₆) δ ppm 8.91 (s, 1H), 7.87 (s, 1H), 7.61-7.54 (m, 2H), 5.60 (t, J=5.6 Hz, 1H), 4.55-4.49 (m, 2H).

6-bromo-7-(((tert-butyldimethylsilyl)oxy)methyl)imidazo[1,2-a]pyridine: To a solution of (6-bromoimidazo[1,2-a]pyridin-7-yl)methanol (590 mg, 2.34 mmol, 1 eq) in DCM (10 mL) was added TBSCl (384.20 mg, 2.55 mmol, 313.64 μL, 1.09 eq) and imidazole (191.05 mg, 2.81 mmol, 1.2 eq) at 0° C. The mixture was stirred at 20° C. for 2 hours before it was diluted with H₂O (10 mL) and extracted with DCM (3×20 mL). The organic phase was separated, dried over Na₂SO₄, filtered and concentrated under reduced pressure to give a residue, which was purified by column chromatography (SiO₂, petroleum ether/EtOAc=1/0 to 1/1) to give the title compound (800 mg, 90% yield) as a white solid. [M+H]⁺=340.8. ¹H NMR (400 MHz, DMSO-d₆) δ ppm 8.94 (s, 1H), 7.89 (s, 1H), 7.61-7.51 (m, 2H), 4.71 (s, 2H), 0.95 (s, 9H), 0.14 (s, 6H).

7-(((tert-butyldimethylsilyl)oxy)methyl)-6-(tert-butylthio)imidazo[1,2-a]pyridine: A mixture of 6-bromo-7-(((tert-butyldimethylsilyl)oxy)methyl)imidazo[1,2-a]pyridine (300 mg, 791.05 μmol, 1 eq), 2-methylpropane-2-thiol (356.71 mg, 3.96 mmol, 445.33 μL, 5 eq), dppf (87.71 mg, 158.21 μmol, 0.2 eq), Pd(OAc)₂ (17.76 mg, 79.11 μmol, 0.1 eq) and t-BuONa (228.07 mg, 2.37 mmol, 3 eq) in dioxane (30 mL) was degassed and purged with N₂ 3 times at 20° C., and then the mixture was stirred at 110° C. for 12 hours under a N₂ atmosphere. The reaction mixture was filtered and concentrated under reduced pressure to give a residue, which was purified by column chromatography (SiO₂, petroleum ether/EtOAc=1/0 to 0/1) to give the title compound (193 mg, 62% yield) as yellow oil. [M+H]⁺=351.1. ¹H NMR (400 MHz, DMSO-d₆) δ ppm 8.75 (s, 1H), 7.97 (s, 1H), 7.60-7.50 (m, 2H), 4.86 (s, 2H), 1.27 (s, 9H), 0.95 (s, 9H), 0.13 (s, 6H).

(6-(tert-butylsulfonyl)imidazo[1,2-a]pyridin-7-yl)methanol: To a solution of 7-(((tert-butyldimethylsilyl)oxy)methyl)-6-(tert-butylthio)imidazo[1,2-a]pyridine (193 mg, 495.45 μmol, 1 eq) in MeOH (6 mL) was added Oxone® (456.87 mg, 743.17 μmol, 1.5 eq). The mixture was stirred at 20° C. for 2 hours before it was concentrated to give the title compound (180 mg,) as a yellow liquid that was used in the next step without purification. [M+H]⁺=268.9.

(6-(tert-butylsulfonyl)-3-iodoimidazo[1,2-a]pyridin-7-yl)methanol: To a solution of (6-(tert-butylsulfonyl)imidazo[1,2-a]pyridin-7-yl)methanol (180 mg, 670.81 μmol, 1 eq) in H₂O (2 mL) was added NIS (226.38 mg, 1.01 mmol, 1.5 eq) at 0° C. The mixture was stirred at 20° C. for 1 hour before it was partitioned between H₂O (5 mL) and DCM (3×10 mL). The organic phase was separated, washed with brine (2×10 mL), dried over Na₂SO₄, filtered and concentrated under reduced pressure to give a residue, which was purified by column chromatography (SiO₂, petroleum ether/EtOAc=1/0 to 0/1) to give the title compound (135 mg, 51% yield) as a white solid. [M+H]⁺=394.9. H NMR (400 MHz, DMSO-d₆) δ ppm 8.51 (s, 1H), 7.92-7.83 (m, 2H), 5.64 (t, J=5.6 Hz, 1H), 4.89-4.83 (m, 2H), 1.32 (s, 9H).

### Intermediate 49

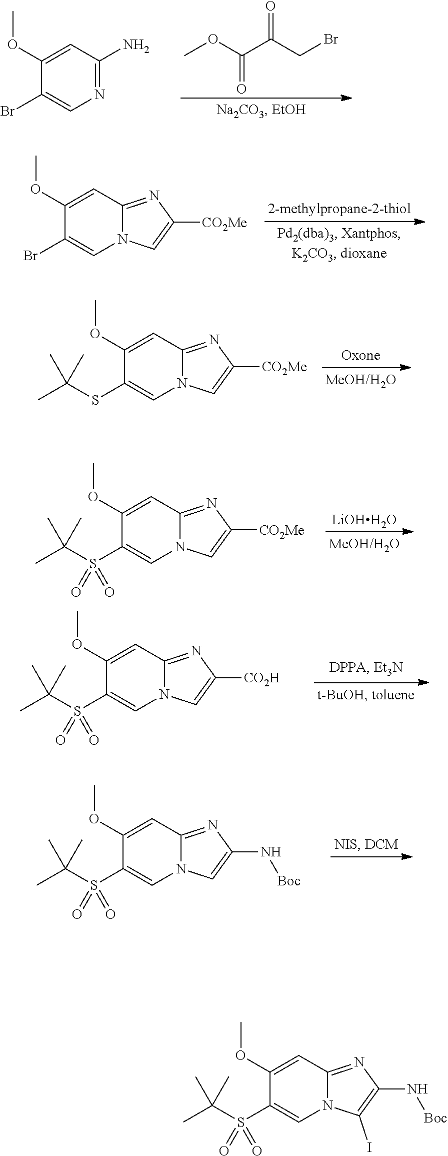

methyl 6-bromo-7-methoxyimidazo[1,2-a]pyridine-2-carboxylate: To a solution of 5-bromo-4-methoxypyridin-2-amine (500 mg, 2.46 mmol, 1 eq) in EtOH (50 mL) was added Na₂CO₃ (783.03 mg, 7.39 mmol, 3 eq) and methyl 3-bromo-2-oxopropanoate (891.39 mg, 4.93 mmol, 524.35 μL, 2 eq). The mixture was stirred at 80° C. for 12 hours before it was concentrated under reduced pressure to give a residue, which was purified by column chromatography (SiO₂, DCM/MeOH=100/0 to 92/8) to give the title compound (560 mg, 36% yield) as a brown solid. ¹H NMR (400 MHz, DMSO-d₆) δ ppm 8.91 (s, 1H), 8.30 (s, 1H), 7.10 (s, 1H), 3.93 (s, 3H), 3.82 (s, 3H).

methyl 6-(tert-butylthio)-7-methoxyimidazo[1,2-a]pyridine-2-carboxylate: A mixture of 2-methylpropane-2-thiol (239.15 mg, 2.65 mmol, 298.57 μL, 1.5 eq), methyl 6-bromo-7-methoxyimidazo[1,2-a]pyridine-2-carboxylate (560 mg, 1.77 mmol, 1 eq), Pd₂(dba)₃ (161.88 mg, 176.78 μmol, 0.1 eq), Xantphos (204.58 mg, 353.57 μmol, 0.2 eq) and K₂CO₃ (732.98 mg, 5.30 mmol, 3 eq) in 1,4-dioxane (30 mL) was degassed and purged with N₂ 3 times, and then was stirred at 90° C. for 12 hours under a N₂ atmosphere. The reaction mixture was concentrated under reduced pressure to give a residue, which was purified by column chromatography (SiO₂, DCM/MeOH=1:0 to 10:1) to give the title compound (510 mg, 78% yield) as a yellow solid. [M+H]⁺=295.0.

methyl 6-(tert-butylsulfonyl)-7-methoxyimidazo[1,2-a]pyridine-2-carboxylate: A mixture of methyl 6-(tert-butylthio)-7-methoxyimidazo[1,2-a]pyridine-2-carboxylate (510 mg, 1.39 mmol, 1 eq) and Oxone® (2.56 g, 4.16 mmol, 3 eq) in H₂O (5 mL) and MeOH (15 mL) was stirred at 25° C. for 12 hours under a N₂ atmosphere before it was filtered and concentrated under reduced pressure to give a residue. The residue was diluted with H₂O (20 mL) and extracted with DCM (3×20 mL), dried over Na₂SO₄, filtered and concentrated under reduced pressure to give a residue, which was purified by column chromatography (SiO₂, DCM/MeOH=1:0 to 10:1) to give the title compound (380 mg, 76% yield) as a yellow solid. ¹H NMR (400 MHz, DMSO-d₆) δ ppm 9.19 (s, 1H), 8.57 (s, 1H), 7.16 (s, 1H), 3.91 (s, 3H), 3.84 (s, 3H), 1.31 (s, 9H).

6-(tert-butylsulfonyl)-7-methoxyimidazo[1,2-a]pyridine-2-carboxylic acid: A mixture of methyl 6-(tert-butylsulfonyl)-7-methoxyimidazo[1,2-a]pyridine-2-carboxylate (380 mg, 1.05 mmol, 1 eq) and LiOH·H₂O (439.74 mg, 10.48 mmol, 10 eq) in H₂O (4 mL) and MeOH (16 mL) was stirred at 25° C. for 1 hour under a N₂ atmosphere. The mixture was concentrated under reduced pressure and the residue was dissolved in H₂O (10 mL), adjusted to pH 5 with 1N HCl, then filtered and give the title compound (240 mg, 66% yield) as a yellow solid. ¹H NMR (400 MHz, DMSO-d₆) δ ppm 9.17 (s, 1H), 8.51 (s, 1H), 7.14 (s, 1H), 3.91 (s, 3H), 1.31 (s, 9H).

tert-butyl (6-(tert-butylsulfonyl)-7-methoxyimidazo[1,2-a]pyridin-2-yl)carbamate: A mixture of 6-(tert-butylsulfonyl)-7-methoxyimidazo[1,2-a]pyridine-2-carboxylic acid (240 mg, 691.55 μmol, 1 eq), Et₃N (83.97 mg, 829.86 μmol, 115.51 μL, 1.2 eq) and DPPA (228.38 mg, 829.86 μmol, 179.12 μL, 1.2 eq) in toluene (8 mL) and t-BuOH (8 mL) was degassed and purged with N₂ 3 times before it was stirred at 90° C. for 12 hours under a N₂ atmosphere. The reaction mixture was concentrated under reduced pressure to give a residue that was purified by column chromatography (SiO₂, DCM/MeOH=1/0 to 20/1) to give the title compound (140 mg, 48% yield) as a yellow solid. ¹H NMR (400 MHz, DMSO-d₆) δ ppm 10.12-9.79 (m, 1H), 9.03 (s, 1H), 7.84 (s, 1H), 6.98 (s, 1H), 3.86 (s, 3H), 1.48 (s, 9H), 1.28 (s, 9H).

tert-butyl (6-(tert-butylsulfonyl)-3-iodo-7-methoxyimidazo[1,2-a]pyridin-2-yl)carbamate: To a mixture of tert-butyl (6-(tert-butylsulfonyl)-7-methoxyimidazo[1,2-a]pyridin-2-yl)carbamate (100 mg, 234.70, μmol, 1 eq) in DCM (10 mL) was added NIS (63.37 mg, 281.64 μmol, 1.2 eq) at 0° C. The mixture was degassed and purged with N₂ 3 times, and then it was stirred at 0° C. for 0.5 hour under a N₂ atmosphere. The reaction mixture was quenched with sat. Na₂SO₃ (10 mL) before it was diluted with H₂O (10 mL) and extracted with DCM (2×20 mL), dried over Na₂SO₄, filtered and concentrated under reduced pressure to give the title compound (120 mg, 90% yield) as a yellow solid. H NMR (400 MHz, DMSO-d₆) δ ppm 9.23 (s, 1H), 8.46 (s, 1H), 7.18 (s, 1H), 3.92 (s, 3H), 1.46 (s, 9H), 1.32 (s, 9H).

### Intermediate 50

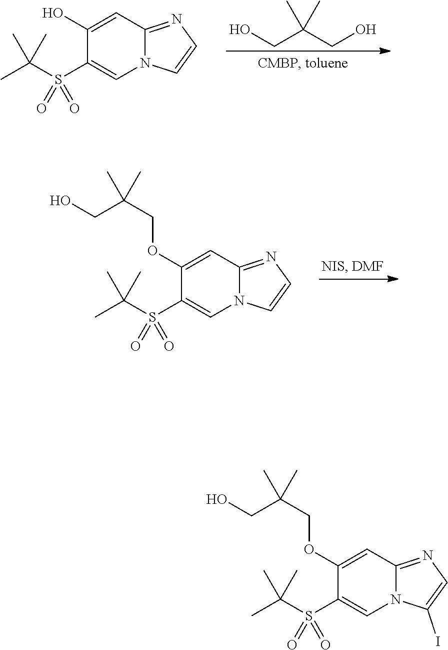

3-((6-(tert-butylsulfonyl)imidazo[1,2-a]pyridin-7-yl)oxy)-2,2-dimethylpropan-1-ol: To a solution of 2,2-dimethylpropane-1,3-diol (55.29 mg, 530.86 μmol, 1 eq) and 6-(tert-butylsulfonyl)imidazo[1,2-a]pyridin-7-ol (150 mg, 530.86 μmol, 1 eq) in toluene (3 mL) was added CMBP (384.37 mg, 1.59 mmol, 3 eq). The mixture was stirred at 100° C. for 2 hours under a N₂ atmosphere before it was diluted with water (3 mL) at 25° C., and then extracted with EtOAc (3×5 mL). The combined organic layers were washed with brine (10 mL), dried over Na₂SO₄, filtered and concentrated under reduced pressure to give a residue, which was purified by column chromatography (SiO₂, DCM/MeOH=100/1 to 95/5) to give the title compound (127 mg, 63% yield) as a brown solid. ¹H NMR (400 MHz, DMSO-d₆) δ ppm 9.14-9.09 (m, 1H), 7.98 (s, 1H), 7.53 (d, J=1.2 Hz, 1H), 7.09 (s, 1H), 4.55 (t, J=5.2 Hz 1H), 3.84 (s, 2H), 3.31 (s, 2H), 1.31 (s, 9H), 0.96 (s, 6H).

3-((6-(tert-butylsulfonyl)-3-iodoimidazo[1,2-a]pyridin-7-yl)oxy)-2,2-dimethylpropan-1-ol: To a solution of 3-((6-(tert-butylsulfonyl)imidazo[1,2-a]pyridin-7-yl)oxy)-2,2-dimethylpropan-1-ol (100 mg, 264.37 μmol, 1 eq) in DMF (2 mL) was added NIS (89.22 mg, 396.55 μmol, 1.5 eq). The mixture was stirred at 25° C. for 2 hours under a N₂ atmosphere before it was quenched with sat. Na₂SO₃ (5 mL) at 25° C., then the reaction mixture was diluted with H₂O (3 mL) and extracted with EtOAc (3×10 mL). The combined organic layers were washed with brine (15 mL×3), dried over Na₂SO₄, filtered and concentrated under reduced pressure to give the title compound (110 mg) as a yellow solid, which was used in the next step without further purification. ¹H NMR (400 MHz, DMSO-d₆) δ ppm 8.47 (s, 1H), 7.71 (s, 1H), 7.24 (s, 1H), 4.57 (s, 1H), 3.88 (s, 2H), 3.31-3.30 (m, 2H), 1.33 (s, 9H), 0.96 (s, 6H).

### Intermediate 51

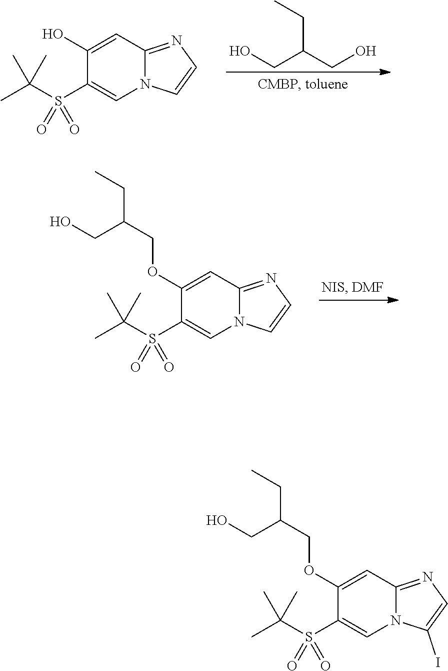

2-(((6-(tert-butylsulfonyl)imidazo[1,2-a]pyridin-7-yl)oxy)methyl)butan-1-ol: A mixture of 2-ethylpropane-1,3-diol (100 mg, 960.18 μmol, 1 eq), 6-(tert-butylsulfonyl)imidazo[1,2-a]pyridin-7-ol (271.31 mg, 960.18 μmol, 1 eq) and CMBP (695.22 mg, 2.88 mmol, 3 eq) in toluene (3 mL) was degassed and purged with N₂ 3 times, and then the mixture was stirred at 100° C. for 2 hours under a N₂ atmosphere. The reaction mixture was concentrated under reduced pressure to give a residue, which was purified by column chromatography (SiO₂, petroleum ether/EtOAc=99/1 to 10/90) to give the title compound (110 mg, 24% yield) as a yellow solid. ¹H NMR (400 MHz, DMSO-d₆) δ ppm 9.12 (s, 1H), 7.98 (s, 1H), 7.53 (s, 1H), 7.12 (s, 1H), 4.49 (t, J=5.2 Hz, 1H), 4.06 (d, J=5.6 Hz, 2H), 3.52 (t, J=5.6 Hz, 2H), 1.80-1.74 (m, 1H), 1.60-1.59 (m, 2H), 1.31 (s, 9H), 0.91-0.89 (m, 3H).

2-(((6-(tert-butylsulfonyl)-3-iodoimidazo[1,2-a]pyridin-7-yl)oxy)methyl)butan-1-ol: A mixture of 2-(((6-(tert-butylsulfonyl)imidazo[1,2-a]pyridin-7-yl)oxy)methyl)butan-1-ol (100 mg, 205.62 μmol, 1 eq) and NIS (55.51 mg, 246.74 μmol, 1.2 eq) in DMF (2 mL) was stirred at 25° C. for 1 hour before it was concentrated under reduced pressure to give a residue, which was purified by column chromatography (SiO₂, DCM/MeOH=99/1 to 86/14) to give the title compound (70 mg, 66% yield) as a yellow solid. [M+H]⁺=466.9. ¹H NMR (400 MHz, DMSO-d₆) δ ppm 8.49 (s, 1H), 7.71 (s, 1H), 7.27 (s, 1H), 4.51 (t, J=5.2 Hz, 1H), 4.10-4.00 (m, 2H), 3.52 (t, J=5.2 Hz, 2H), 1.83-1.74 (m, 1H), 1.49-1.43 (m, 2H), 1.33 (s, 9H), 0.91 (t, J=7.2 Hz, 3H).

### Intermediate 52

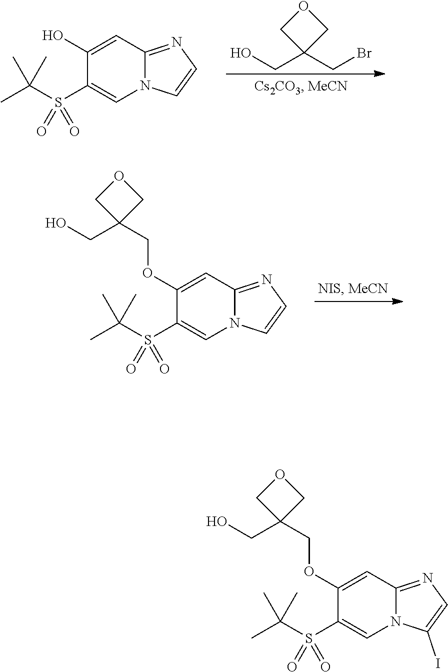 [SMILES: CC(C)(C)S(=O)(=O)c1cn2ccnc2cc1O ; OCC1(CBr)COC1 ; CC(C)(C)S(=O)(=O)c1cn2ccnc2cc1OCC1(CO)COC1 ; CC(C)(C)S(=O)(=O)c1cn2c(I)cnc2cc1OCC1(CO)COC1]

(3-(((6-(tert-butylsulfonyl)imidazo[1,2-a]pyridin-7-yl)oxy)methyl)oxetan-3-yl)methanol: To a solution of (3-(bromomethyl)oxetan-3-yl)methanol (200 mg, 1.10 mmol, 1.2 eq) and 6-(tert-butylsulfonyl)imidazo[1,2-a]pyridin-7-ol (260.15 mg, 920.67 μmol, 1 eq) in MeCN (1 mL) was added Cs₂CO₃ (899.92 mg, 2.76 mmol, 3 eq). The mixture was stirred at 80° C. for 2 hours before it was concentrated under reduced pressure to give a residue, which was purified by column chromatography (SiO₂, DCM/MeOH=100/0 to 10/1) to yield the title compound (200 mg, 55% yield) as a white solid. ¹H NMR (400 MHz, DMSO-d₆) δ ppm 9.14 (s, 1H), 8.00 (s, 1H), 7.55 (d, J=1.2 Hz, 1H), 7.20 (s, 1H), 4.96 (t, J=5.6 Hz, 1H), 4.47-4.37 (m, 4H), 4.30 (s, 2H), 3.76 (d, J=5.2 Hz, 2H), 1.29 (s, 9H).

(3-(((6-(tert-butylsulfonyl)-3-iodoimidazo[1,2-a]pyridin-7-yl)oxy)methyl)oxetan-3-yl)methanol: To a solution of (3-(((6-(tert-butylsulfonyl)imidazo[1,2-a]pyridin-7-yl)oxy)methyl)oxetan-3-yl)methanol (10 mg, 279.33 μmol, 1 eq) in MeCN (5 mL) was added NIS (87.98 mg, 391.06 μmol, 1.4 eq). The mixture was stirred at 20° C. for 2 hours before it was concentrated under reduced pressure to give a residue, which was purified by column chromatography (SiO₂, DCM/MeOH=100/0 to 10/1) to yield the title compound (120 mg, 81% yield) as a light yellow solid. ¹H NMR (400 MHz, DMSO-d₆) δ ppm 8.49 (s, 1H), 7.73 (s, 1H), 7.35 (s, 1H), 4.98 (t, J=5.6 Hz, 1H), 4.44-4.40 (m, 4H), 4.34 (s, 2H), 3.76 (d, J=5.2 Hz, 2H), 1.31 (s, 9H).

### Intermediate 53

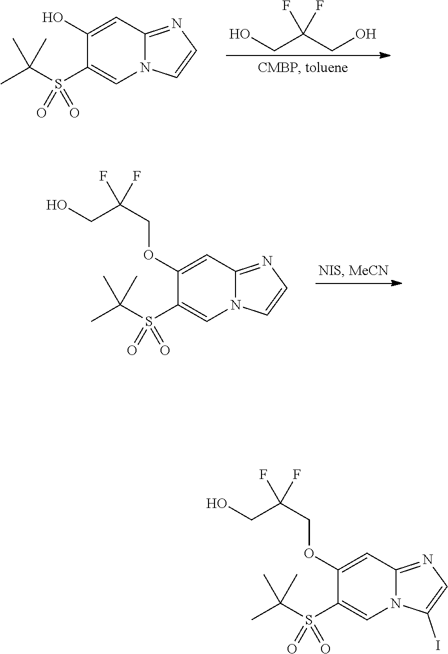 [SMILES: CC(C)(C)S(=O)(=O)c1cn2ccnc2cc1O ; OCC(F)(F)CO ; CC(C)(C)S(=O)(=O)c1cn2ccnc2cc1OCC(F)(F)CO ; CC(C)(C)S(=O)(=O)c1cn2c(I)cnc2cc1OCC(F)(F)CO]

3-((6-(tert-butylsulfonyl)imidazo[1,2-a]pyridin-7-yl)oxy)-2,2-difluoropropan-1-ol: A mixture of 2,2-difluoropropane-1,3-diol (594.96 mg, 5.31 mmol, 5 eq), 6-(tert-butylsulfonyl)imidazo[1,2-a]pyridin-7-ol (300 mg, 1.06 mmol, 1 eq) and CMBP (1.02 g, 4.25 mmol, 4 eq) in toluene (8 mL) was degassed and purged with N₂ 3 times, and then the mixture was stirred at 100° C. for 12 hours under a N₂ atmosphere. The reaction mixture was filtered and concentrated under reduced pressure to give a residue, which was purified by column chromatography (SiO₂, 0-100% EtOAc/petroleum ether) to give the title compound (230 mg, 56% yield) as a light yellow solid. ¹H NMR (400 MHz, DMSO-d₆) δ ppm 9.17 (s, 1H), 8.02 (d, J=1.2 Hz, 1H), 7.58 (d, J=1.2 Hz, 1H), 7.28 (s, 1H), 5.60 (t, J=6.4 Hz, 1H), 4.44 (t, J=12.0 Hz, 2H), 3.91-3.79 (m, 2H), 1.30 (s, 9H).

3-((6-(tert-butylsulfonyl)-3-iodoimidazo[1,2-a]pyridin-7-yl)oxy)-2,2-difluoropropan-1-ol: A mixture of 3-((6-(tert-butylsulfonyl)imidazo[1,2-a]pyridin-7-yl)oxy)-2,2-difluoropropan-1-ol (150 mg, 387.52 μmol, 1 eq) and NIS (104.62 mg, 465.03 μmol, 1.2 eq) in MeCN (3 mL) was degassed and purged with N₂ 3 times, and then the mixture was stirred at 25° C. for 3 hours under a N₂ atmosphere. The reaction mixture was filtered and concentrated under reduced pressure to give a residue, which was purified by column chromatography (SiO₂, 0-10% MeOH/DCM) to give the title compound (200 mg, 87% yield) as a yellow solid. ¹H NMR (400 MHz, DMSO-d₆) δ ppm 8.50 (s, 1H), 7.76 (s, 1H), 7.42 (s, 1H), 5.62 (t, J=5.2 Hz, 1H), 4.48 (t, J=12.0 Hz, 2H), 3.91-3.77 (m, 2H), 1.32 (s, 9H).

### Intermediate 54

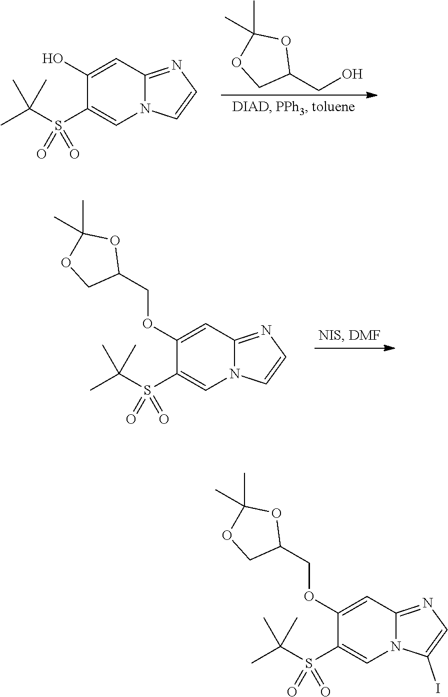

6-(tert-butylsulfonyl)-7-((2,2-dimethyl-1,3-dioxolan-4-yl)methoxy)imidazo[1,2-a]pyridine: To a mixture of (2,2-dimethyl-1,3-dioxolan-4-yl)methanol (100 mg, 756.67 μmol, 93.81 μL, 2 eq) and 6-(tert-butylsulfonyl)imidazo[1,2-a]pyridin-7-ol (106.90 mg, 378.34 μmol, 1 eq) in toluene (2 mL) was added DIAD (153.01 mg, 756.67 μmol, 146.70 μL, 2 eq) and PPh₃ (198.46 mg, 756.67 μmol, 2 eq). The mixture was stirred at 100° C. for 12 hours under N₂ before it was diluted with water (50 mL) and extracted with EtOAc (3×50 mL). The combined organic extracts were dried over Na₂SO₄, filtered and concentrated under reduced pressure to give a residue, which was purified by column chromatography (SiO₂, petroleum ether/EtOAc=100/1 to 0/1) to give the title compound (25 mg, 16% yield) as a yellow solid. ¹H NMR (400 MHz, DMSO-d₆) δ ppm 9.13 (s, 1H), 8.00 (s, 1H), 7.55 (s, 1H), 7.20 (s, 1H), 4.41 (t, J=5.6 Hz, 1H), 4.17 (d, J=5.2 Hz, 2H), 4.12-4.03 (m, 1H), 3.90-3.82 (m, 1H), 1.37 (s, 3H), 1.33-1.28 (m, 12H).

6-(tert-butylsulfonyl)-7-((2,2-dimethyl-1,3-dioxolan-4-yl)methoxy)-3-iodoimidazo[1,2-a]pyridine: To a solution of 6-(tert-butylsulfonyl)-7-((2,2-dimethyl-1,3-dioxolan-4-yl)methoxy)imidazo[1,2-a]pyridine (25 mg, 61.07 μmol, 1 eq) in DMF (2 mL) was added NIS (16.49 mg, 73.28 μmol, 1.2 eq). The mixture was stirred at 25° C. for 2 hours before it was quenched with sat. Na₂SO₃ (20 mL) at 0° C., and then extracted with EtOAc (3×20 mL). The combined organic layers were washed with brine (20 mL), dried over Na₂SO₄, filtered and concentrated under reduced pressure to give the title compound (30 mg, 89% yield) as a yellow solid, which was used in the next step without further purification. ¹H NMR (400 MHz, DMSO-d₆) δ ppm 8.49 (s, 1H), 7.73 (s, 1H), 7.34 (s, 1H), 4.47-4.38 (m, 1H), 4.21 (d, J=4.0 Hz, 2H), 4.13-4.06 (m, 1H), 3.89-3.81 (m, 1H), 1.37 (s, 3H), 1.33 (s, 9H), 1.31 (s, 3H).

### Intermediate 55

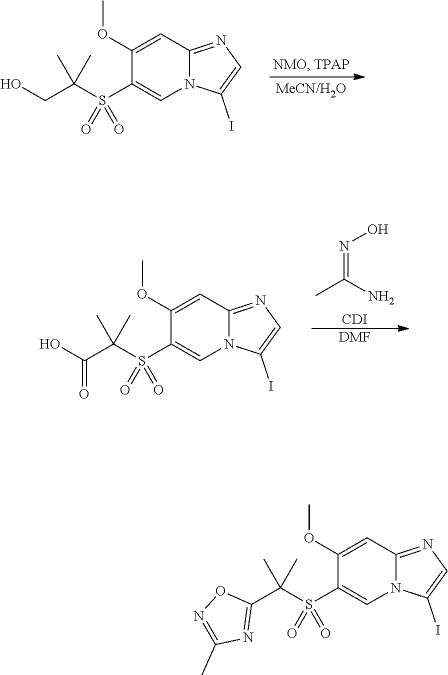

2-((3-iodo-7-methoxyimidazo[1,2-a]pyridin-6-yl)sulfonyl)-2-methylpropanoic acid: To a solution of 2-((3-iodo-7-methoxyimidazo[1,2-a]pyridin-6-yl)sulfonyl)-2-methylpropan-1-ol (100 mg, 219.39 μmol, 1 eq) in MeCN (5 mL) was added NMO (231.55 μL, 2.19 mmol, 10 eq), TPAP (23.13 mg, 65.82 μmol, 0.3 eq) and H₂O (39.53 mg, 2.19 mmol, 39.53 μL, 10 eq). The mixture was stirred at 50° C. for 2 h. After 2 h, the reaction mixture was concentrated in vacuo. The resulting crude material was purified via silica gel chromatography (DCM/MeOH=1/0 to 1/1) to yield the title compound as a black brown solid.

5-(2-((3-iodo-7-methoxyimidazo[1,2-a]pyridin-6-yl)sulfonyl)propan-2-yl)-3-methyl-1,2,4-oxadiazole: To a solution of 2-((3-iodo-7-methoxyimidazo[1,2-a]pyridin-6-yl)sulfonyl)-2-methylpropanoic acid (25 mg, 53.04 μmol, 1 eq) in DMF (2 mL) was added CDI (43.00 mg, 265.20 μmol, 5 eq) at 25° C. The reaction mixture was stirred at room temperature for 1 hour, followed by the addition of N-hydroxyacetamidine (7.86 mg, 106.08 μmol, 2 eq). The resulting mixture was stirred at 80° C. for 11 hours. The reaction mixture was quenched by the addition of H₂O (0.5 mL). The resulting crude material was purified via prep HPLC (acidic conditions) to yield the title compound as a brown solid. ¹H NMR (400 MHz, DMSO-d₆) δ ppm 8.38 (s, 1H), 8.25 (s, 0.5H), 7.75 (s, 1H), 7.20 (s, 1H), 3.66 (s, 3H), 2.34 (s, 3H), 1.82 (s, 6H).

### Intermediate 56

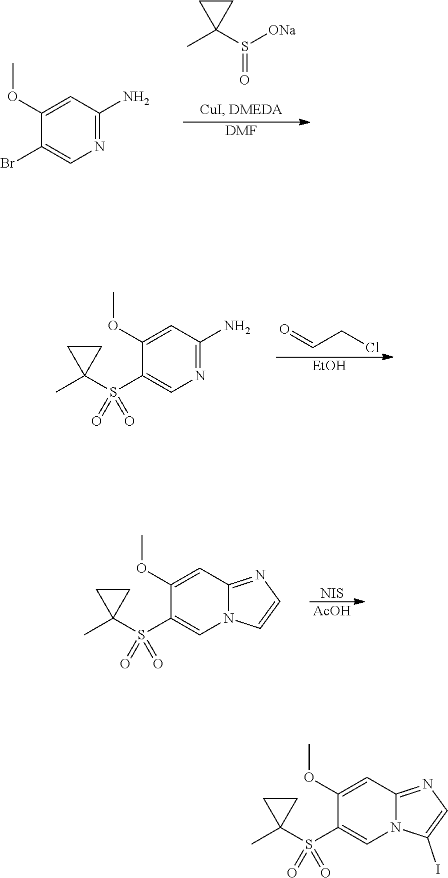

4-methoxy-5-((1-methylcyclopropyl)sulfonyl)pyridin-2-amine: To a stirred solution of 5-bromo-4-methoxypyridin-2-amine (10 mg, 49.3 μmol, 1.0 eq.) in DMF (0.5 mL) was added 1-methylcyclopropane-1-sulfinate sodium salt (17.5 mg, 123 μmol, 2.5 eq.), CuI (1.88 mg, 10.0 μmol, 0.2 eq.) and DMEDA (5.50 mg, 25.0 μmol, 0.5 eq.) at room temperature. The reaction mixture was heated at 120° C. for 16 h before it was filtered through Celite® and rinsed with EtOAc (10 mL). The filtrate was evaporated under reduced pressure to provide the title compound (10.0 mg, 84% yield) as a brown gummy liquid. The compound was used as such in the next step without further purification. [M+H]⁺=243.1.

7-methoxy-6-((1-methylcyclopropyl)sulfonyl)imidazo[1,2-a]pyridine: To an ice cooled solution of 4-methoxy-5-((1-methylcyclopropyl)sulfonyl)pyridin-2-amine (10 mg, 41.3 μmol, 1.0 eq.) in EtOH (1 mL) was added 2-chloroacetaldehyde (16.1 mg, 207 μmol, 5.0 eq.) at room temperature. The reaction mixture was stirred at 80° C. for 16 h before it was evaporated under reduced pressure to provide the title compound (10.0 mg) as a brown gummy liquid. The compound was used as such in next step without further purification. [M+H]⁺=267.3.

3-iodo-7-methoxy-6-((1-methylcyclopropyl)sulfonyl)imidazo[1,2-a]pyridine: To a stirred solution of 7-methoxy-6-((1-methylcyclopropyl)sulfonyl)imidazo[1,2-a]pyridine (12.0 mg, 45.1 μmol, 1.0 eq.) in acetic acid (1 mL) was added NIS (12.2 mg, 54.1 μmol, 1.2 eq.) at 0° C. The reaction mixture was allowed to warm to room temperature and stir there for 1 h before it was diluted with ice cold water (2 mL) and extracted with EtOAc (2×5 mL). The combined organic layers were washed with brine (1 mL), dried over Na₂SO₄, filtered and concentrated under reduced pressure to provide the title compound (15.0 mg) as a pale yellow solid. The compound was used as such in next step without further purification. [M+H]⁺=393.1.

### Intermediate 57

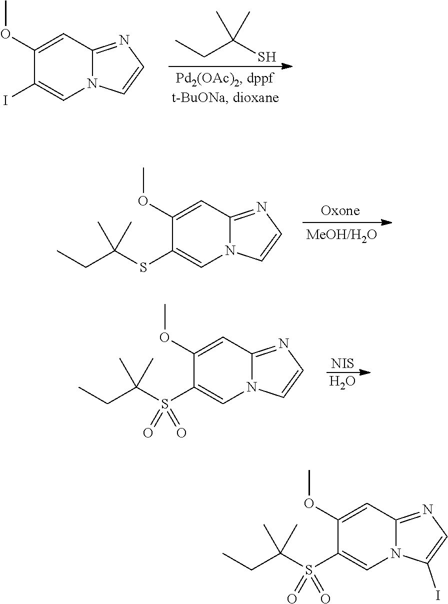

7-methoxy-6-(tert-pentylthio)imidazo[1,2-a]pyridine: To a mixture of 6-iodo-7-methoxyimidazo[1,2-a]pyridine (500 mg, 2.20 mmol, 1 eq), 2-methylbutane-2-thiol (458.97 mg, 4.40 mmol, 2 eq), dppf (244.16 mg, 440.42 μmol, 0.2 eq) and t-BuONa (317.44 mg, 3.30 mmol, 1.5 eq) in dioxane (10 mL) was added Pd(OAc)₂ (49.44 mg, 220.21 μmol, 0.1 eq). The mixture was stirred at 90° C. for 12 hours under N₂ before saturated NH₄Cl (100 mL) was added and the mixture was extracted with EtOAc (2×100 mL). The combined organic layers were washed with brine (2×100 mL), dried over Na₂SO₄, filtered and concentrated under reduced pressure to give a residue, which was purified by column chromatography (SiO₂, 0-8% MeOH/DCM) to give the title compound (410 mg, 52% yield) as a brown solid. [M+H]⁺=251.2. ¹H NMR (400 MHz, CDCl₃) δ ppm 8.23 (s, 1H), 7.48-7.36 (m, 2H), 6.92 (br s, 1H), 3.91 (s, 3H), 1.56 (q, J=7.6 Hz, 2H), 1.23 (s, 6H), 1.02 (t, J=7.6 Hz, 3H)

7-methoxy-6-(tert-pentylsulfonyl)imidazo[1,2-a]pyridine: To a solution of 7-methoxy-6-(tert-pentylthio)imidazo[1,2-a]pyridine (300 mg, 838.80 μmol, 1 eq) in MeOH (6 mL) and H₂O (2 mL) was added Oxone® (1.55 g, 2.52 mmol, 3 eq), and then the mixture was stirred at 15° C. for 12 hours. The reaction mixture was used directly in the next step without work up. The title compound (230 mg, 68% yield) was obtained as yellow liquid. [M+H]⁺=283.2.

3-iodo-7-methoxy-6-(tert-pentylsulfonyl)imidazo[1,2-a]pyridine: To a mixture of 7-methoxy-6-(tert-pentylsulfonyl)imidazo[1,2-a]pyridine (230 mg, 570.20 μmol, 1 eq) in H₂O (2 mL) was added NIS (192.43 mg, 855.30 μmol, 1.5 eq) and the mixture was stirred at 15° C. for 1 hour. Water (80 mL) was added and the mixture was extracted with DCM (2×80 mL). The combined organic layers were washed with saturated Na₂SO₃ (2×80 mL) and brine (80 mL×2), dried over Na₂SO₄, filtered and concentrated under reduced pressure to give a residue that was purified by column chromatography (SiO₂, 0-5% MeOH/DCM) to give the title compound (220 mg, 90% yield) as a white solid. [M+H]⁺=409.1. ¹H NMR (400 MHz, CDCl₃) δ ppm 8.70 (s, 1H), 7.68 (s, 1H), 7.13 (s, 1H), 4.01 (s, 3H), 1.83 (q, J=7.6 Hz, 2H), 1.38 (s, 6H), 1.02 (t, J=7.6 Hz, 3H).

### Intermediate 58

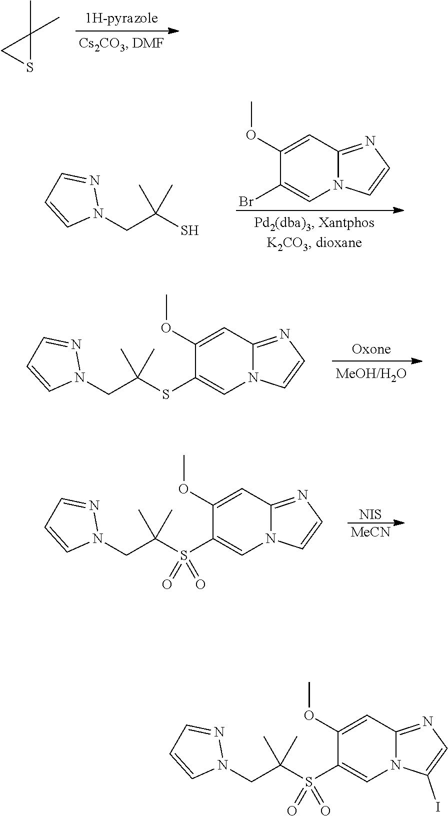

2-methyl-1-(1H-pyrazol-1-yl)propane-2-thiol: To a solution of 2,2-dimethylthiirane (1.94 g, 22.03 mmol, 1.5 eq) in DMF (20 mL) was added Cs₂CO₃ (14.36 g, 44.07 mmol, 3 eq) and 1H-pyrazole (1 g, 14.69 mmol, 1 eq). The mixture was stirred at 80° C. for 12 hours under a N₂ atmosphere before it was filtered, diluted with H₂O (100 mL) and extracted with EtOAc (3×100 mL). The combined organic layers were washed with brine (100 mL), dried over Na₂SO₄, filtered and concentrated under reduced pressure to give a residue, which was purified by column chromatography (SiO₂, petroleum ether/EtOAc=100/0 to 4/1) to give the title compound (260 mg, 10% yield) as colorless oil. ¹H NMR (400 MHz, CDCl₃) δ ppm 7.51 (d, J=1.6 Hz, 1H), 7.42 (d, J=2.4 Hz, 1H), 6.26 (t, J=1.6 Hz, 1H), 4.21 (s, 2H), 1.32 (s, 6H).

7-methoxy-6-((2-methyl-1-(1H-pyrazol-1-yl)propan-2-yl)thio)imidazo[1,2-a]pyridine: A mixture of 2-methyl-1-(1H-pyrazol-1-yl)propane-2-thiol (100 mg, 576.01 μmol, 1 eq), 6-bromo-7-methoxyimidazo[1,2-a]pyridine (145.32 mg, 576.01 μmol, 1 eq), Pd₂(dba)₃ (105.49 mg, 115.20 μmol, 0.2 eq), K₂CO₃ (238.82 mg, 1.73 mmol, 3 eq) and Xantphos (133.32 mg, 230.40 μmol, 0.4 eq) in 1,4-dioxane (4 mL) was degassed and purged with N₂ 3 times. The mixture was stirred at 100° C. for 12 hours under a N₂ atmosphere before it was concentrated under reduced pressure to give a residue, which was purified by column chromatography (SiO₂, 0-5% MeOH/DCM) to give the title compound (115 mg, 59% yield) as brown oil. ¹H NMR (400 MHz, CDCl₃) δ ppm 8.35 (s, 1H), 7.57-7.48 (m, 3H), 7.44 (s, 1H), 6.94 (s, 1H), 6.27 (t, J=2.0 Hz, 1H), 4.25 (s, 2H), 3.93 (s, 3H), 1.26 (s, 6H).

7-methoxy-6-((2-methyl-1-(1H-pyrazol-1-yl)propan-2-yl)sulfonyl)imidazo[1,2-a]pyridine: To a mixture of 7-methoxy-6-((2-methyl-1-(1H-pyrazol-1-yl)propan-2-yl)thio)imidazo[1,2-a]pyridine (95 mg, 282.74 μmol, 1 eq) in MeOH (4 mL) and H₂O (1 mL) was added Oxone® (1.04 g, 1.70 mmol, 6 eq). The mixture was stirred at 20° C. for 2 hours before it was diluted with H₂O (5 mL) and extracted with DCM (3×10 mL). The combined organic extracts were washed with brine (2×10 mL), dried over Na₂SO₄, filtered and concentrated under reduced pressure to give a residue, which was purified by column chromatography (SiO₂, 0-15% MeOH/DCM) to give the title compound (85 mg, 76% yield) as a yellow oil. ¹H NMR (400 MHz, CDCl₃)) δ ppm 8.76 (s, 1H), 7.64 (s, 1H), 7.57 (s, 1H), 7.46 (d, J=2.4 Hz, 2H), 7.08-6.98 (m, 1H), 6.27 (t, J=2.0 Hz, 1H), 4.56 (s, 2H), 3.98 (s, 3H), 1.40 (s, 6H).

3-iodo-7-methoxy-6-((2-methyl-1-(1H-pyrazol-1-yl)propan-2-yl)sulfonyl)imidazo[1,2-a]pyridine: To a mixture of 7-methoxy-6-((2-methyl-1-(1H-pyrazol-1-yl)propan-2-yl)sulfonyl)imidazo[1,2-a]pyridine (80 mg, 191.39 μmol, 1 eq) in MeCN (8 mL) was added NIS (34.45 mg, 153.11 μmol, 0.8 eq) at 0° C. The mixture was stirred at 0° C. for 2 hours before it was diluted with H₂O (10 mL) and extracted with DCM (3×20 mL). The combined organic extracts were washed with brine (2×30 mL), dried over Na₂SO₄, filtered and concentrated under reduced pressure to give the title compound (80 mg) as a yellow solid, which was used for the next step without purification. ¹H NMR (400 MHz, CDCl₃) δ ppm 8.69 (s, 1H), 7.68 (s, 1H), 7.49-7.43 (m, 2H), 7.08 (s, 1H), 6.26 (t, J=2.4 Hz, 1H), 4.56 (s, 2H), 4.00 (s, 3H), 1.43 (s, 6H).

### Intermediate 59

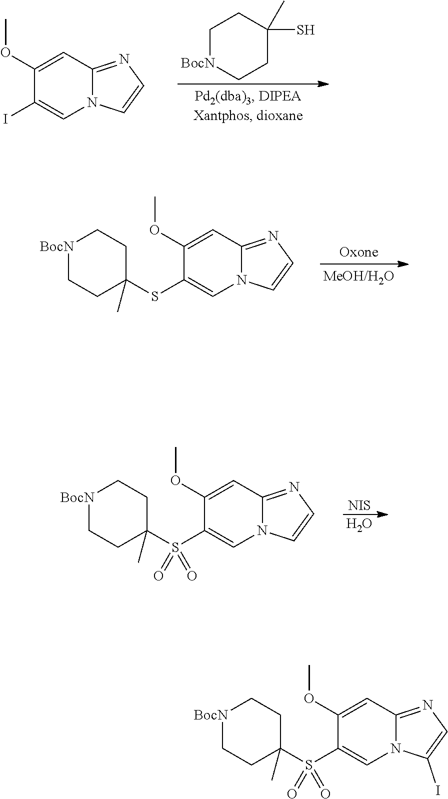

tert-butyl 4-((7-methoxyimidazo[1,2-a]pyridin-6-yl)thio)-4-methylpiperidine-1-carboxylate: A mixture of 4-methyl-4-sulfanyl-piperidine-1-carboxylic acid tert-butyl ester (730 mg, 2.84 mmol, 1 eq), 6-iodo-7-methoxyimidazo[1,2-a]pyridine (864.74 mg, 2.84 mmol, 1 eq), DIPEA (1.10 g, 8.52 mmol, 1.48 mL, 3 eq), Xantphos (657.26 mg, 1.14 mmol, 0.4 eq) and Pd₂(dba)₃ (520.09 mg, 567.96 μmol, 0.2 eq) in dioxane (20 mL) was degassed and purged with N₂ (3×). The resulting reaction mixture was stirred at 100° C. for 12 h under a N₂ atmosphere before it was filtered and concentrated in vacuo. The resulting crude material was purified by column chromatography (SiO₂, EtOAc) to yield the title compound as a yellow oil. LCMS [M+H]⁺=378.1. ¹H NMR (400 MHz, DMSO-d₆) δ ppm 8.22 (s, 1H), 7.51 (d, J=1.2 Hz, 1H), 7.43 (s, 1H), 6.93 (s, 1H), 3.91 (s, 3H), 3.65-3.48 (m, 4H), 1.72-1.64 (m, 2H), 1.60-1.51 (m, 2H), 1.45 (s, 9H), 1.26 (s, 3H).

tert-butyl 4-((7-methoxyimidazo[1,2-a]pyridin-6-yl)sulfonyl)-4-methylpiperidine-1-carboxylate: To a solution of tert-butyl 4-((7-methoxyimidazo[1,2-a]pyridin-6-yl)thio)-4-methylpiperidine-1-carboxylate (400 mg, 953.64 μmol, 1 eq) in MeOH (8 mL) and H₂O (2 mL) was added Oxone® (1.17 g, 1.91 mmol, 2 eq). The mixture was stirred at 25° C. for 12 h. The title compound was obtained as a light yellow liquid, which was used for the next step without work up or purification. LCMS [M+H]⁺=410.0.

tert-butyl 4-((3-iodo-7-methoxyimidazo[1,2-a]pyridin-6-yl)sulfonyl)-4-methylpiperidine-1-carboxylate: A mixture of tert-butyl 4-((7-methoxyimidazo[1,2-a]pyridin-6-yl)sulfonyl)-4-methylpiperidine-1-carboxylate (400 mg, 976.80 μmol, 1 eq) and NIS (329.65 mg, 1.47 mmol, 1.5 eq) in H₂O (2 mL) was stirred at 25° C. for 1 h before it was quenched with saturated NaHSO₃ (2 mL) at 0° C. The mixture was diluted with H₂O (50 mL) and extracted with DCM (3×20 mL). The combined organic layers were dried over Na₂SO₄, filtered and concentrated in vacuo. The resulting crude material was purified by column chromatography (SiO₂, DCM/MeOH=19/1 to 93/7) to yield the title compound as a yellow solid. LCMS [M+H]⁺=536.0. ¹H NMR (400 MHz, DMSO-d₆) δ ppm 8.66 (s, 1H), 7.67 (s, 1H), 7.10-7.03 (m, 1H), 4.19-4.06 (m, 2H), 3.98 (s, 3H), 2.98-2.86 (m, 2H), 2.21-2.08 (m, 2H), 1.72-1.64 (m, 2H), 1.45 (s, 12H).

### Intermediate 60

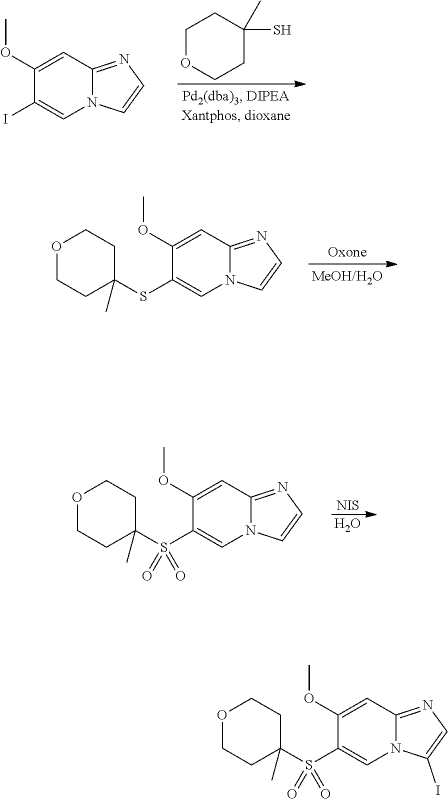

7-methoxy-6-((4-methyltetrahydro-2H-pyran-4-yl)thio)imidazo[1,2-a]pyridine: A mixture of 6-iodo-7-methoxyimidazo[1,2-a]pyridine (500 mg, 1.64 mmol, 1 eq), 4-methyltetrahydro-2H-pyran-4-thiol (241.23 mg, 1.64 mmol, 1 eq), DIPEA (636.65 mg, 4.93 mmol, 858.02 μL, 3 eq), Xantphos (380.03 mg, 656.80 μmol, 0.4 eq) and Pd₂(dba)₃ (300.72 mg, 328.40 μmol, 0.2 eq) in dioxane (20 mL) was degassed and purged with N₂ 3 times. The mixture was stirred at 100° C. for 12 hours under a N₂ atmosphere before it was diluted with H₂O (10 mL) and extracted with EtOAc (2×10 mL). The combined organic layers were washed with brine (10 mL), dried over Na₂SO₄, filtered and concentrated under reduced pressure to give a residue, which was purified by column chromatography (SiO₂, DCM/MeOH=100/0 to 92/8) to give the title compound (340 mg, 67% yield) as yellow oil. ¹H NMR (400 MHz, DMSO-d₆) δ ppm 8.70 (s, 1H), 7.77 (s, 1H), 7.42 (s, 1H), 6.98 (s, 1H), 3.84 (s, 3H), 3.80-3.74 (m, 2H), 3.62-3.57 (m, 2H), 1.61-1.53 (m, 4H), 1.24 (s, 3H).

7-methoxy-6-((4-methyltetrahydro-2H-pyran-4-yl)sulfonyl)imidazo[1,2-a]pyridine: To a solution of 7-methoxy-6-((4-methyltetrahydro-2H-pyran-4-yl)thio)imidazo[1,2-a]pyridine (310 mg, 1.00 mmol, 1 eq) in MeOH (12 mL) and H₂O (3 mL) was added Oxone® (1.23 g, 2.00 mmol, 2 eq). The mixture was stirred at 25° C. for 12 hours to give the title compound (310 mg) as a light yellow liquid that was used in next step directly.

3-iodo-7-methoxy-6-((4-methyltetrahydro-2H-pyran-4-yl)sulfonyl)imidazo[1,2-a]pyridine: To a solution of 7-methoxy-6-((4-methyltetrahydro-2H-pyran-4-yl)sulfonyl)imidazo[1,2-a]pyridine (310 mg, 998.81 μmol, 1 eq) in H₂O (4 mL) was added NIS (337.08 mg, 1.50 mmol, 1.5 eq). The mixture was stirred at 25° C. for 1 hour before it was quenched with saturated Na₂SO₃ (2 mL), diluted with H₂O (10 mL) and extracted with EtOAc (2×10 mL). The combined organic layer was washed with brine (10 mL), dried over Na₂SO₄, and concentrated under reduced pressure to give a residue, which was purified by column chromatography (SiO₂, DCM/MeOH=100/0 to 93/7) to give the title compound (320 mg, 66% yield) as a yellow solid. [M+H]⁺=436.8. ¹H NMR (400 MHz, DMSO-d₆) δ ppm 8.49 (s, 1H), 7.73 (s, 1H), 7.28 (s, 1H), 3.92 (s, 3H), 3.86-3.81 (m, 2H), 3.47 (t, J=11.2 Hz, 2H), 2.12-2.01 (m, 2H), 1.55-1.46 (m, 2H), 1.41 (s, 3H).

### Intermediate 61

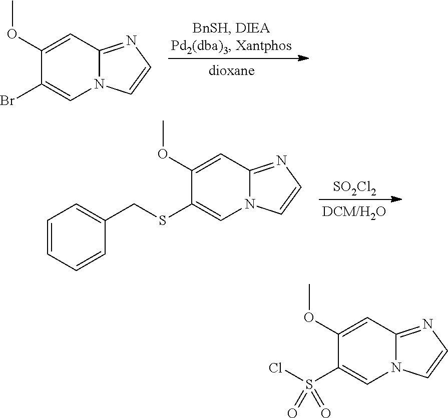

6-(benzylthio)-7-methoxyimidazo[1,2-a]pyridine: A mixture of 6-bromo-7-methoxyimidazo[1,2-a]pyridine (3 g, 11.89 mmol, 1 eq), phenylmethanethiol (2.95 g, 23.8 mmol, 2.79 mL, 2 eq), Pd₂(dba)₃ (1.09 g, 1.19 mmol, 0.1 eq), Xantphos (1.38 g, 2.38 mmol, 0.2 eq) and DIEA (4.61 g, 35.7 mmol, 6.21 mL, 3 eq) in 1,4-dioxane (30 mL) was degassed and purged with N₂ three times, and then the mixture was stirred at 90° C. for 12 h under N₂. The reaction mixture was concentrated under reduced pressure to give a residue, which was purified by column chromatography (SiO₂, 0-6% MeOH/DCM) to give the title compound (3.0 g, 84% yield) as a brown oil. ¹H NMR (400 MHz, CDCl₃) δ ppm 7.86 (s, 1H), 7.44 (s, 1H), 7.28 (s, 1H), 7.24-7.17 (m, 3H), 7.16-7.12 (m, 2H), 6.93 (s, 1H), 4.00 (s, 2H), 3.96 (s, 3H).

7-methoxyimidazo[1,2-a]pyridine-6-sulfonyl chloride: To a solution of 6-(benzylthio)-7-methoxyimidazo[1,2-a]pyridine (500 mg, 1.66 mmol, 1 eq) in DCM (6 mL) was added H₂O (1.2 mL) and sulfuryl chloride (1.57 g, 11.7 mmol, 1.16 mL, 7 eq) at 0° C. The mixture was stirred at 20° C. for 0.5 h. The title compound (500 mg) was obtained as yellow liquid, which was used directly. [M+H]⁺=247.1.

### Intermediate 62

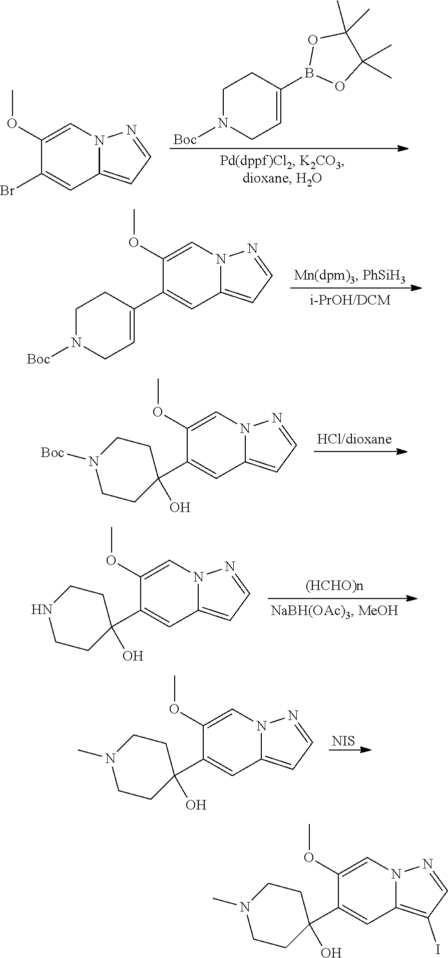

tert-butyl 4-(6-methoxypyrazolo[1,5-a]pyridin-5-yl)-3,6-dihydropyridine-1 (2H)-carboxylate: To a solution of 5-bromo-6-methoxypyrazolo[1,5-a]pyridine (462.01 mg, 1.63 mmol, 1 eq) and tert-butyl 4-(4,4,5,5-tetramethyl-1,3,2-dioxaborolan-2-yl)-3,6-dihydropyridine-1 (2H)-carboxylate (755 mg, 2.44 mmol, 1.5 eq) in dioxane (5 mL) and H₂O (1 mL) was added Pd(dppf)Cl₂ (119.11 mg, 162.78 μmol, 0.1 eq) and K₂CO₃ (674.92 mg, 4.88 mmol, 3 eq). The mixture was stirred at 80° C. for 12 h under a N₂ atmosphere before it was filtered and concentrated in vacuo. The resulting crude material was purified by column chromatography (SiO₂, PE/EtOAc=1/0 to 2/1) to yield the title compound as a yellow solid. ¹H NMR (400 MHz, DMSO-d₆) δ ppm 8.35 (s, 1H), 7.84 (d, J=2.0 Hz, 1H), 7.44 (s, 1H), 6.49 (d, J=2.4 Hz, 1H), 5.95 (s, 1H), 3.98 (s, 2H), 3.82 (s, 3H), 3.50 (t, J=5.6 Hz, 2H), 2.41 (s, 2H), 1.43 (s, 9H).

tert-butyl 4-hydroxy-4-(6-methoxypyrazolo[1,5-a]pyridin-5-yl)piperidine-1-carboxylate: A mixture of tert-butyl 4-(6-methoxypyrazolo[1,5-a]pyridin-5-yl)-3,6-dihydropyridine-1 (2H)-carboxylate (400 mg, 1.09 mmol, 1 eq), Mn(dppm)₃ (66.09 mg, 109.29 μmol, 0.1 eq) and PhSiH₃ (236.54 mg, 2.19 mmol, 269.71 μL, 2 eq) in iPrOH (10 mL) and DCM (2 mL) was degassed and purged with 02 (3×). The reaction mixture was stirred at 25° C. for 1 h under a 02 atmosphere (15 psi) before it was diluted with H₂O (10 mL) and extracted with EtOAc (3×10 mL). The combined organic layers were washed with brine (10 mL), dried over Na₂SO₄, filtered and concentrated in vacuo. The resulting crude material was purified by column chromatography (SiO₂, PE/EtOAc=1/0 to 0/1) to yield the title compound as a brown oil. 11H NMR (400 MHz, CDCl₃) δ ppm 8.34 (s, 1H), 7.88 (d, J=2.4 Hz, 1H), 7.48 (s, 1H), 6.51 (d, J=2.0 Hz, 1H), 4.07-4.01 (m, 2H), 3.96 (s, 3H), 3.38-3.24 (m, 2H), 2.02-1.92 (m, 4H), 1.48 (s, 9H).

4-(6-methoxypyrazolo[1,5-a]pyridin-5-yl)piperidin-4-ol: A mixture of tert-butyl 4-hydroxy-4-(6-methoxypyrazolo[1,5-a]pyridin-5-yl)piperidine-1-carboxylate (100 mg, 259.06 μmol, 1 eq) in HCl/dioxane (4 M, 11.25 mL, 173.70 eq) was stirred at 25° C. for 2 h. The reaction mixture concentrated in vacuo and the crude material was used in the next step without further purification. LCMS [M+H]⁺=248.2.

4-(6-methoxypyrazolo[1,5-a]pyridin-5-yl)-1-methylpiperidin-4-ol: To a solution of 4-(6-methoxypyrazolo[1,5-a]pyridin-5-yl)piperidin-4-ol (90 mg, 363.94 μmol, 1 eq) in MeOH (5 mL) was added paraformaldehyde (90.00 mg, 3.00 mmol, 8.23 eq) and AcOH (125.01 μL, 2.18 mmol, 6 eq) at 25° C. The resulting mixture was stirred at 25° C. for 1 h, and then NaBH(OAc)₃ (68.61 mg, 1.09 mmol, 3 eq) was added. The resulting mixture was stirred at 25° C. for 3 h before it was quenched with H₂O (0.5 mL) and concentrated in vacuo. The resulting crude material was used in the next step without further purification. LCMS [M+H]⁺=262.2.

4-(3-iodo-6-methoxypyrazolo[1,5-a]pyridin-5-yl)-1-methylpiperidin-4-ol: To a solution of 4-(6-methoxypyrazolo[1,5-a]pyridin-5-yl)-1-methylpiperidin-4-ol (95 mg, 363.54 μmol, 1 eq) in MeOH (10 mL) and H₂O (10 mL) was added NIS (654.33 mg, 2.91 mmol, 8 eq). The mixture was stirred at 25° C. for 12 h before it was extracted with EtOAc (3×20 mL). The combined organic layers were washed with brine (20 mL), dried over Na₂SO₄, filtered and concentrated in vacuo. The resulting crude material was purified by column chromatography (SiO₂, DCM/MeOH=1/0 to 9/1) to yield the title compound as a yellow solid. ¹H NMR (400 MHz, DMSO-d₆) δ ppm 9.25 (s, 1H), 8.51 (s, 1H), 7.99 (s, 1H), 7.67 (s, 1H), 3.94-3.85 (m, 3H), 2.85 (s, 3H), 2.84-2.74 (m, 2H), 2.73-2.68 (m, 2H), 2.33 (s, 2H), 1.69-1.52 (m, 2H).

### Intermediate 63

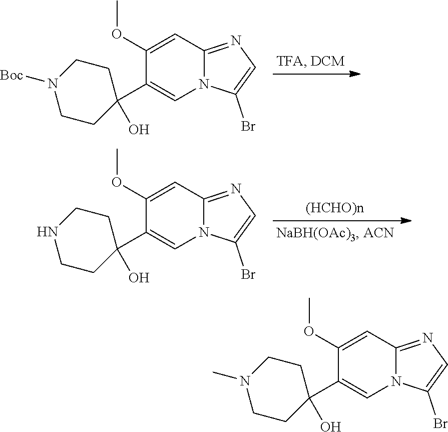

4-(3-bromo-7-methoxyimidazo[1,2-a]pyridin-6-yl)piperidin-4-ol: To a mixture of tert-butyl 4-(3-bromo-7-methoxyimidazo[1,2-a]pyridin-6-yl)-4-hydroxypiperidine-1-carboxylate (570 mg, 1.20 mmol, 1 eq) in DCM (2 mL) was added TFA (4.37 g, 38.37 mmol, 2.85 mL, 31.88 eq) at 25° C. The mixture was stirred at 25° C. for 1 hour under a N₂ atmosphere before it was concentrated under reduced pressure to give the title compound (200 mg, 46% yield) as yellow oil. ¹H NMR (400 MHz, DMSO-d₆) δ ppm 8.30 (s, 1H), 7.53 (s, 1H), 7.07 (s, 1H), 5.58 (s, 1H), 3.90 (s, 3H), 3.16-3.00 (m, 4H), 2.65-2.56 (m, 2H), 1.55-1.42 (m, 2H).

4-(3-bromo-7-methoxyimidazo[1,2-a]pyridin-6-yl)-1-methylpiperidin-4-ol: To a mixture of 4-(3-bromo-7-methoxyimidazo[1,2-a]pyridin-6-yl)piperidin-4-ol (200 mg, 551.83 μmol, 1 eq), paraformaldehyde (200 mg, 2.21 mmol, 4 eq) in MeCN (4 mL) was added NaBH(OAc)₃ (467.82 mg, 2.21 mmol, 4 eq) at 0° C. The mixture was stirred at 25° C. for 6 hours under a N₂ atmosphere before it was filtered and concentrated under reduced pressure to give the title compound (120 mg, 58% yield) as a yellow oil, which was used in the next step without further purification. ¹H NMR (400 MHz, DMSO-d₆) δ ppm 8.31 (s, 1H), 7.54 (s, 1H), 7.08 (s, 1H), 6.52 (s, 1H), 3.91 (s, 3H), 2.75-2.60 (m, 6H), 2.56 (s, 3H), 1.55-1.40 (m, 2H).

### Intermediate 64

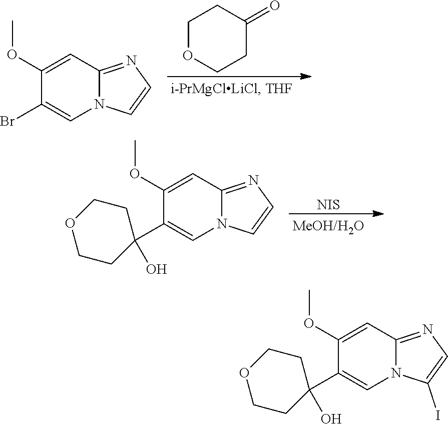

4-(7-methoxyimidazo[1,2-a]pyridin-6-yl)tetrahydro-2H-pyran-4-ol: To a solution of 6-bromo-7-methoxyimidazo[1,2-a]pyridine (1 g, 3.96 mmol, 1 eq) in THF (15 mL) was added i-PrMgCl·LiCl (1.3 M, 9.15 mL, 3 eq) at −10° C. The mixture was stirred at −10° C. for 30 min, and then tetrahydro-4H-pyran-4-one (1.19 g, 11.89 mmol, 1.09 mL, 3 eq) was added. The resulting mixture was stirred at 25° C. for 2 hours under a N₂ atmosphere before it was quenched with saturated NH₄Cl at 0° C. and extracted with EtOAc (3×100 mL). The combined organic layers were washed with brine (160 mL), dried over Na₂SO₄, filtered and concentrated under reduced pressure to give the residue, which was purified by column chromatography (SiO₂, 0-10% MeOH/DCM) to give the title compound (220 mg, 18% yield) as a brown solid. ¹H NMR (400 MHz, DMSO-d₆) δ ppm 8.55 (s, 1H), 7.88 (s, 1H), 7.42 (s, 1H), 7.03 (s, 1H), 5.22 (s, 1H), 3.87 (s, 3H), 3.85-3.74 (m, 2H), 3.72-3.62 (m, 2H), 2.46-2.43 (m, 2H), 1.41-1.31 (m, 2H).

4-(3-iodo-7-methoxyimidazo[1,2-a]pyridin-6-yl)tetrahydro-2H-pyran-4-ol: To a solution of 4-(7-methoxyimidazo[1,2-a]pyridin-6-yl)tetrahydro-2H-pyran-4-ol (100.00 mg, 322.22 μmol, 1 eq) in MeOH (3 mL) and H₂O (2 mL) was added NIS (108.74 mg, 483.33 μmol, 1.5 eq). The mixture was stirred at 25° C. for 3 hours before it was concentrated, diluted with water (30 mL), and then extracted with EtOAc (3×30 mL). The combined organic layers were washed with brine (50 mL), dried over Na₂SO₄, filtered and concentrated to give the residue, which was purified by column chromatography (SiO₂, 0-10% MeOH/DCM) to give the title compound (50 mg, 37% yield) as a brown solid. [M+H]⁺=374.9. H NMR (400 MHz, DMSO-d₆) δ ppm 8.34 (s, 1H), 7.51 (s, 1H), 7.05 (s, 1H), 5.44 (s, 1H), 3.91 (s, 3H), 3.85-3.62 (m, 4H), 2.67-2.51 (m, 2H), 1.33 (d, J=13.2 Hz, 2H).

### Intermediate 65

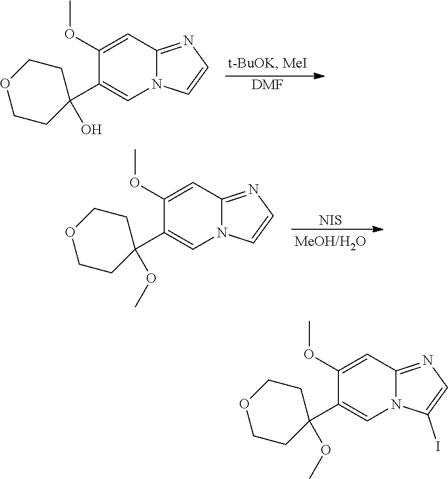

7-methoxy-6-(4-methoxytetrahydro-2H-pyran-4-yl)imidazo[1,2-a]pyridine: To a solution of 4-(7-methoxyimidazo[1,2-a]pyridin-6-yl)tetrahydro-2H-pyran-4-ol (230 mg, 833.75 μmol, 1 eq) in DMF (15 mL) was added t-BuOK (561.33 mg, 5.00 mmol, 6 eq) and Mel (236.68 mg, 1.67 mmol, 103.81 μL, 2 eq) at 0° C. The mixture was stirred at 0° C. for 1 hour before it was filtered and concentrated under reduced pressure to give the residue, which was purified by column chromatography (SiO₂, 0-10% MeOH/DCM) to give the title compound (60 mg, 25% yield) as a yellow solid. [M+H]⁺=262.9. ¹H NMR (400 MHz, CDCl₃) δ ppm 7.95 (s, 1H), 7.50 (s, 1H), 7.44 (s, 1H), 7.13-6.91 (m, 1H), 3.92 (s, 3H), 3.89-3.80 (m, 4H), 3.14 (s, 3H), 2.29-2.23 (m, 2H), 2.18-2.02 (m, 2H).

3-iodo-7-methoxy-6-(4-methoxytetrahydro-2H-pyran-4-yl)imidazo[1,2-a]pyridine: To a solution of 7-methoxy-6-(4-methoxytetrahydro-2H-pyran-4-yl)imidazo[1,2-a]pyridine (60 mg, 205.87 μmol, 1 eq) in MeOH (4 mL) and H₂O (2 mL) was added NIS (69.47 mg, 308.80 μmol, 1.5 eq). The mixture was stirred at 25° C. for 3 hours before it was concentrated, diluted with water (30 mL), and then extracted with EtOAc (3×30 mL). The combined organic layers were washed with brine (50 mL), dried over Na₂SO₄, filtered and concentrated to give the title compound (65 mg, 73% yield) as a white solid that was used in the next step without further purification. [M+H]⁺=388.9. ¹H NMR (400 MHz, DMSO-d₆) δ ppm 7.90 (s, 1H), 7.54 (s, 1H), 7.09 (s, 1H), 3.88 (s, 3H), 3.79-3.61 (m, 4H), 3.07 (s, 3H), 2.28-2.11 (m, 2H), 2.10-2.01 (m, 2H).

### Intermediate 66

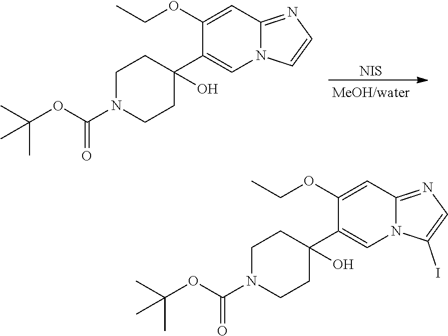

tert-Butyl 4-(7-ethoxy-3-iodoimidazo[1,2-a]pyridin-6-yl)-4-hydroxypiperidine-1-carboxylate: To tert-butyl 4-(7-ethoxyimidazo[1,2-a]pyridin-6-yl)-4-hydroxypiperidine-1-carboxylate (1.83 g, 5.06 mmol, prepared according to US20180072717 A1) in MeOH (20 mL) and water (8 mL) was added N-iodosuccinimide (1.37 g, 6.08 mmol). The mixture was stirred at 20° C. for 30 min before aqueous sodium thiosulfate solution (10%) and water were added. The precipitate was collected by filtration, washed with water and dried to give 2.22 g (89% yield) of the title compound, which was used without further purification. [M+H]⁺=488.5. ¹H NMR (400 MHz, DMSO-d₆) δ ppm 8.34 (s, 1H), 7.51 (s, 1H), 7.00 (s, 1H), 5.52 (s, 1H), 4.03-4.19 (m, 2H), 3.73-3.98 (m, 2H), 2.97-3.28 (m, 2H), 1.42 (s, 9H), 1.30-1.39 (m, 5H).

### Intermediate 67

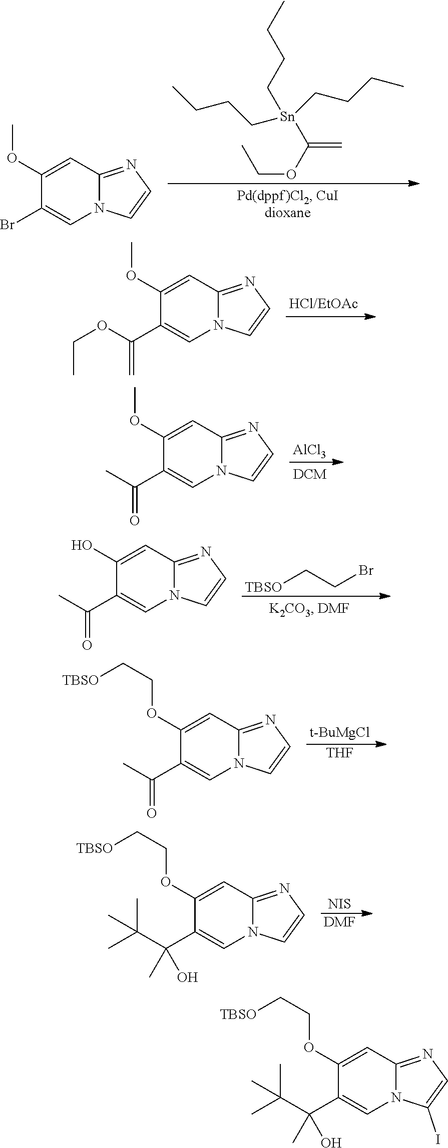

6-(1-ethoxyvinyl)-7-methoxyimidazo[1,2-a]pyridine: A mixture of 6-bromo-7-methoxyimidazo[1,2-a]pyridine (5 g, 19.82 mmol, 1 eq), tributyl(1-ethoxyvinyl)stannane (14.32 g, 39.64 mmol, 13.39 mL, 2 eq), Pd(dppf)Cl₂ (1.45 g, 1.98 mmol, 0.1 eq) and CuI (377.45 mg, 1.98 mmol, 0.1 eq) in dioxane (50 mL) was degassed and purged with N₂ for 3 times, and then the mixture was stirred at 110° C. for 12 hours under N₂ atmosphere. The reaction mixture was diluted with H₂O (100 mL) and extracted with EtOAc (100 mL×3). The combined organic layers were washed with brine (50 mL), dried over Na₂SO₄, filtered and concentrated under reduced pressure to give a residue, which was purified by column chromatography (SiO₂, 0-5% MeOH in DCM) to give the title compound (4 g, 83% yield) as a brown oil. ¹H NMR (400 MHz, DMSO-d₆) δ ppm 8.99-8.36 (m, 1H), 7.68-7.33 (m, 2H), 7.19-6.86 (m, 1H), 4.65 (d, J=1.6 Hz, 1H), 4.47 (d, J=1.6 Hz, 1H), 3.92-3.77 (m, 5H), 1.30 (t, J=6.8 Hz, 3H).

1-(7-methoxyimidazo[1,2-a]pyridin-6-yl)ethan-1-one: To a solution of 6-(1-ethoxyvinyl)-7-methoxyimidazo[1,2-a]pyridine (3.8 g, 15.67 mmol, 1 eq) in EtOAc (20 mL) was added HCl (12 M, 1.90 mL, 1.46 eq). The mixture was stirred at 25° C. for 30 minutes. The reaction mixture was concentrated under reduced pressure to give the title compound (4 g) as a brown solid, which was used into the next step without further purification. 1-(7-hydroxyimidazo[1,2-a]pyridin-6-yl)ethan-1-one: To a solution of 1-(7-methoxyimidazo[1,2-a]pyridin-6-yl)ethan-1-one in DCE (30 mL) was added AlCl₃ (10.52 g, 78.87 mmol, 4.31 mL, 5 eq). The mixture was stirred at 80° C. for 1 hour. The reaction mixture was quenched by the addition of sat. Na₂SO₄·10H₂O (20 mL), then the pH was adjusted to 7 with sat. NaHCO₃. The mixture was then extracted with DCM (120 mL), and the organic phase was dried over Na₂SO₄, filtered and concentrated under reduced pressure to give a residue, which was purified by column chromatography (SiO₂, 0-10% MeOH in DCM) to give the title compound as a yellow solid. ¹H NMR (400 MHz, DMSO-d₆) δ ppm 11.39-11.05 (m, 1H), 9.42-9.13 (m, 1H), 7.90-7.72 (m, 1H), 7.48 (s, 1H), 6.86-6.60 (m, 1H), 2.65 (s, 3H).

1-(7-(2-((tert-butyldimethylsilyl)oxy)ethoxy)imidazo[1,2-a]pyridin-6-yl)ethan-1-one: To a mixture of (2-bromoethoxy)(tert-butyl)dimethylsilane (1.34 g, 5.62 mmol, 1.1 eq) and 1-(7-hydroxyimidazo[1,2-a]pyridin-6-yl)ethan-1-one (1 g, 5.11 mmol, 1 eq) in DMF (10 mL) was added K₂CO₃ (1.06 g, 7.66 mmol, 1.5 eq). The mixture was stirred at 60° C. for 2 hours. The reaction mixture was diluted with H₂O (50 mL) and extracted with EtOAc (3×50 mL). The combined organic layers were washed with brine (100 mL), dried over Na₂SO₄, filtered and concentrated under reduced pressure to give a residue, which was purified by column chromatography (SiO₂, 0-50% EtOAc in PE) to yield the title compound (1 g, 53% yield) as a yellow solid. ¹H NMR (400 MHz, DMSO-d₆) δ ppm 8.86 (s, 1H), 7.87 (s, 1H), 7.47 (d, J=1.2 Hz, 1H), 7.08-7.01 (m, 1H), 4.23-4.18 (m, 2H), 4.01-3.98 (m, 2H), 2.60 (s, 3H), 0.86 (s, 9H), 0.06 (s, 6H).

2-(7-(2-((tert-butyldimethylsilyl)oxy)ethoxy)imidazo[1,2-a]pyridin-6-yl)-3,3-dimethylbutan-2-ol: To a solution of 1-(7-(2-((tert-butyldimethylsilyl)oxy)ethoxy)imidazo[1,2-a]pyridin-6-yl)ethan-1-one (400 mg, 1.08 mmol, 1 eq) in THF (20 mL) was added t-BuMgCl (2 M, 5.38 mL, 10 eq) at 0° C. The mixture was stirred at 0° C. for 1 hour under N₂. The reaction mixture was quenched by addition sat. NH₄Cl (10 mL) at 0° C., and then diluted with H₂O (20 mL) and extracted with EtOAc (3×20 mL). The combined organic layers were dried over Na₂SO₄, filtered and concentrated under reduced pressure to give a residue, which was purified by column chromatography (SiO₂, 0-5% MeOH in DCM) to yield the title compound (50 mg, 10% yield) as a yellow solid. ¹H NMR (400 MHz, DMSO-d₆) δ ppm 8.50 (s, 1H), 7.81 (s, 1H), 7.35 (d, J=1.2 Hz, 1H), 6.89-6.81 (m, 1H), 4.91 (s, 1H), 4.11-4.05 (m, 1H), 4.05-3.99 (m, 1H), 3.96-3.92 (m, 2H), 1.66 (s, 3H), 0.89 (s, 18H), 0.09 (s, 6H).

2-(7-(2-((tert-butyldimethylsilyl)oxy)ethoxy)-3-iodoimidazo[1,2-a]pyridin-6-yl)-3,3-dimethylbutan-2-ol: To a solution of 2-(7-(2-((tert-butyldimethylsilyl)oxy)ethoxy)imidazo[1,2-a]pyridin-6-yl)-3,3-dimethylbutan-2-ol (90 mg, 206.31 μmol, 1 eq) in DMF (2 mL) was added NIS (69.63 mg, 309.47 μmol, 1.5 eq). The mixture was stirred at 25° C. for 1 hour. The reaction mixture was diluted with H₂O (30 mL) and extracted with EtOAc (3×20 mL). The combined organic layers were washed with brine (50 mL), dried over Na₂SO₄, filtered and concentrated under reduced pressure to give a residue which was purified by column chromatography (SiO₂, 0-20% EtOAc/petroleum ether) to yield the title compound (100 mg, 84% yield) as a yellow solid. ¹H NMR (400 MHz, DMSO-d₆) δ ppm 8.33 (s, 1H), 7.50 (s, 1H), 6.97 (s, 1H), 5.06 (s, 1H), 4.13-4.08 (m, 1H), 4.06-4.01 (m, 1H), 3.96-3.92 (m, 2H), 1.69 (s, 3H), 0.90-0.86 (m, 18H), 0.08 (s, 6H).

### Intermediate 68

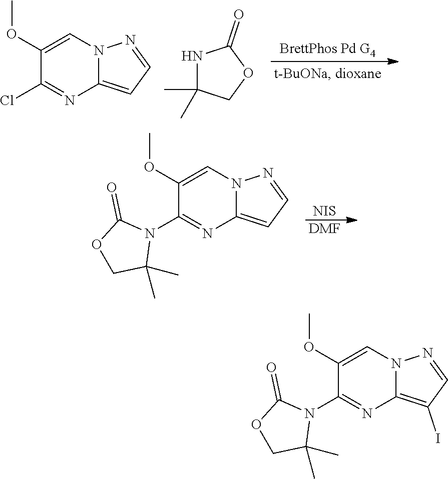

3-(6-methoxypyrazolo[1,5-a]pyrimidin-5-yl)-4,4-dimethyloxazolidin-2-one: To a mixture of 5-chloro-6-methoxypyrazolo[1,5-a]pyrimidine (50 mg, 245.10 μmol, 1 eq) and 4,4-dimethyloxazolidin-2-one (42.33 mg, 367.66 μmol, 1.5 eq) in 1,4-dioxane (2 mL) was added BrettPhos Pd G4 (22.56 mg, 24.51 μmol, 0.1 eq) and t-BuONa (70.67 mg, 735.31 μmol, 3 eq). The mixture was degassed and purged with N₂ 3 times, and then it stirred at 100° C. for 2 hours under a N₂ atmosphere. The reaction mixture was concentrated under reduced pressure, diluted with EtOAc (10 mL) and washed with H₂O (3×10 mL). The combined organic phase was washed with brine (3×10 mL), dried over Na₂SO₄, filtered and concentrated under reduced pressure to give the title compound (43 mg) as a red solid, which was used in the next step without further purification. ¹H NMR (400 MHz, DMSO-d₆) δ ppm 9.13-8.96 (m, 1H), 8.13 (s, 1H), 6.72-6.64 (m, 1H), 3.96 (s, 3H), 3.94 (s, 2H), 1.21 (s, 6H).

3-(3-iodo-6-methoxypyrazolo[1,5-a]pyrimidin-5-yl)-4,4-dimethyloxazolidin-2-one: To a solution of 3-(6-methoxypyrazolo[1,5-a]pyrimidin-5-yl)-4,4-dimethyloxazolidin-2-one (30 mg, 114.39 μmol, 1 eq) in DMF (1 mL) was added NIS (30.88 mg, 137.27 μmol, 1.2 eq). The mixture was stirred at 20° C. for 2 hours before it was quenched with saturated Na₂SO₃ (10 mL). The mixture was diluted with EtOAc (20 mL), washed with H₂O (3×10 mL) and brine (3×10 mL), dried over Na₂SO₄, filtered and concentrated under reduced pressure. The residue was purified by column chromatography (SiO₂, 0-25% EtOAc/petroleum ether) to yield the title compound (40 mg, 72% yield) as a white solid. ¹H NMR (400 MHz, DMSO-d₆) δ ppm 9.10 (s, 1H), 8.22 (s, 1H), 4.28 (s, 2H), 3.88 (s, 3H), 1.22 (s, 6H).

### Intermediate 69

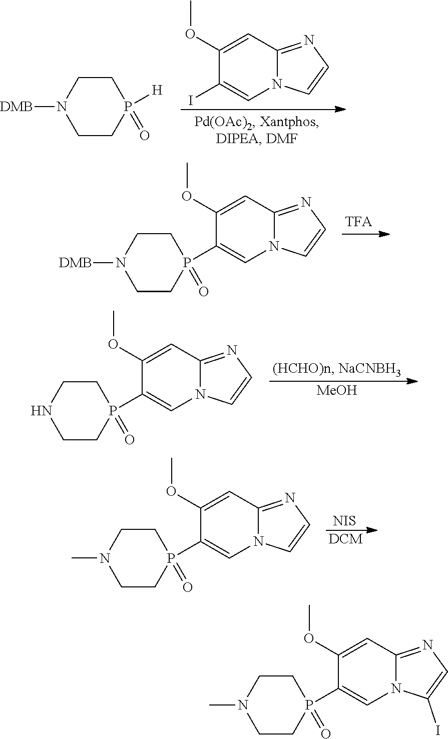

1-(2,4-dimethoxybenzyl)-4-(7-methoxyimidazo[1,2-a]pyridin-6-yl)-1,4-azaphosphinane 4-oxide: A mixture of 6-iodo-7-methoxyimidazo[1,2-a]pyridine (280 mg, 919.51 μmol, 1 eq), 1-(2,4-dimethoxybenzyl)-1,4-azaphosphinane 4-oxide (550.23 mg, 1.84 mmol, 2 eq), Pd(OAc)₂ (20.64 mg, 91.95 μmol, 0.1 eq), Xantphos (106.41 mg, 183.90 μmol, 0.2 eq) and DIPEA (356.52 mg, 2.76 mmol, 480.49 μL, 3 eq) in DMF (20 mL) was degassed and purged with N₂ 3 times. The mixture was stirred at 90° C. for 12 hours under a N₂ atmosphere before it was concentrated under reduced pressure to give a residue, which was purified by column chromatography (SiO₂, 0-8% MeOH/DCM) to give the title compound (320 mg, 75% yield) as a light yellow solid. [M+H]⁺=416.2. ¹H NMR (400 MHz, DMSO-d₆) δ ppm 8.89-8.80 (m, 1H), 7.95 (s, 1H), 7.48 (s, 1H), 7.25 (d, J=8.4 Hz, 1H), 7.12-6.96 (m, 1H), 6.57-6.50 (m, 2H), 3.94 (s, 3H), 3.78 (s, 3H), 3.76 (s, 3H), 3.66 (s, 2H), 3.09-2.97 (m, 2H), 2.92-2.82 (m, 2H), 2.48-2.41 (m, 2H), 1.85-1.73 (m, 2H).

4-(7-methoxyimidazo[1,2-a]pyridin-6-yl)-1,4-azaphosphinane 4-oxide: A mixture of 1-(2,4-dimethoxybenzyl)-4-(7-methoxyimidazo[1,2-a]pyridin-6-yl)-1,4-azaphosphinane 4-oxide (320 mg, 693.27 μmol, 1 eq) in TFA (10 mL) was degassed and purged with N₂ 3 times, and then the mixture was stirred at 70° C. for 12 hours under a N₂ atmosphere. The reaction mixture was concentrated under reduced pressure to give the title compound (180 mg) as a brown solid, which was used in the next step without further purification.

4-(7-methoxyimidazo[1,2-a]pyridin-6-yl)-1-methyl-1,4-azaphosphinane 4-oxide: A mixture of 4-(7-methoxyimidazo[1,2-a]pyridin-6-yl)-1,4-azaphosphinane 4-oxide (180 mg, 678.61 μmol, 1 eq), AcOH (4.20 g, 69.87 mmol, 4 mL, 102.96 eq), paraformaldehyde (360 mg, 2.71 mmol, 4 eq) in MeOH (10 mL) was stirred at 25° C. for 1 hours, and then NaBH₃CN (170.58 mg, 2.71 mmol, 4 eq) was added at 0° C. The resulting mixture was stirred at 25° C. for 1 hour under a N₂ atmosphere before it was filtered and concentrated under reduced pressure to give a residue, which was purified by RP-HPLC to give the title compound (150 mg, 71% yield) as a light yellow oil. [M+H]⁺=280.2. ¹H NMR (400 MHz, DMSO-d₆) δ ppm 8.84-8.78 (m, 1H), 7.94 (s, 1H), 7.47 (d, J=0.8 Hz, 1H), 7.06 (d, J=4.4 Hz, 1H), 3.91 (s, 3H), 3.04-2.85 (m, 2H), 2.81-2.64 (m, 2H), 2.46-2.35 (m, 2H), 2.31 (s, 3H), 1.89-1.74 (m, 2H).

4-(3-iodo-7-methoxyimidazo[1,2-a]pyridin-6-yl)-1-methyl-1,4-azaphosphinane 4-oxide: To a solution of 4-(7-methoxyimidazo[1,2-a]pyridin-6-yl)-1-methyl-1,4-azaphosphinane 4-oxide (110 mg, 393.88 μmol, 1 eq) in DCM (10 mL) was added NIS (88.62 mg, 393.88 μmol, 1 eq) at 0° C. The mixture was stirred at 0° C. for 10 min before it was concentrated under reduced pressure to give a residue. The crude product was purified by RP-HPLC to give the title compound (5 mg) as a white solid.

### Intermediate 70

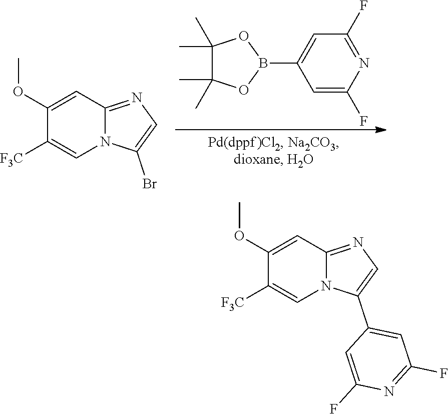

3-(2,6-Difluoropyridin-4-yl)-7-methoxy-6-(trifluoromethyl)imidazo[1,2-a]pyridine: A solution of 3-bromo-7-methoxy-6-(trifluoromethyl)imidazo[1,2-a]pyridine (70 mg, 0.237 mmol) and 2,6-difluoro-4-(4,4,5,5-tetramethyl-1,3,2-dioxaborolan-2-yl)pyridine (68.621 mg, 0.285 mmol) in 1,4-dioxane (1.12 ml) was degassed with nitrogen for 5 min, and then Na₂CO₃ (50.290 mg, 0.474 mmol) and PdCl₂(dppf)·DCM complex (17.363 mg, 0.024 mmol) and water (0.38 ml) were added to the reaction mixture. The resulting mixture was heated at 100° C. for 2 h before it was diluted with water (5 mL) and extracted with 10% MeOH/DCM (2×10 mL). The combined organic layer was dried over Mg₂SO₄ filtered and concentrated under vacuum. The residue was purified by column chromatography (SiO₂, 40-60% EtOAc/petroleum ether) to provide the title compound (65 mg, 83% yield) as an off-white solid. [M+H]⁺=330.4.

The following compounds were prepared following procedures analogous to that described for Intermediate 70.

| Int. | | | |
|---|---|---|---|
| No. | Structure | Name | LCMS,¹H NMR |
| 71 | 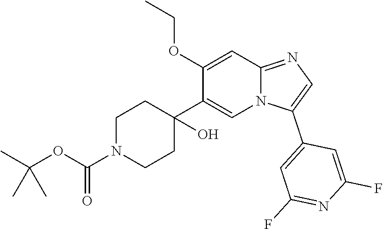 [SMILES: CCOc1cc2ncc(-c3cc(F)nc(F)c3)n2cc1C1(O)CCN(C(=O)OC(C)(C)C)CC1] | tert-butyl 4-(3-(2,6- difluoropyridin-4-yl)- 7-ethoxyimidazo[1,2- a]pyridin-6-yl)-4- hydroxypiperidine-1- carboxylate | [M + H]⁺ = 475.5 ¹H NMR (400 MHZ, DMSO-d₆) δ ppm 8.74 (s, 1 H), 8.12 (s, 1 H), 7.51 (s, 2 H), 7.14 (s, 1 H), 5.54 (s, 1 H), 4.10 - 4.23 (m, 2 H), 3.77 - 4.00 (m, 2 H), 3.00 - 3.29 (m, 2 H), 1.43 (s, 9 H), 1.37 (s, 5 H). |
| 72 | 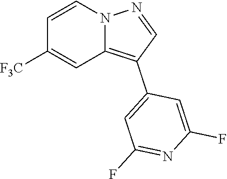 [SMILES: Fc1cc(-c2cnn3ccc(C(F)(F)F)cc23)cc(F)n1] | 3-(2,6- difluoropyridin-4-yl)- 5-(trifluoromethyl) pyrazolo[1,5-a]pyridine | ¹H NMR (400 MHZ, CDCl₃) δ ppm 8.67 (d, J = 7.2 Hz, 1H), 8.36 (s, 1H), 8.12 (s, 1H), 7.11 (dd, J = 7.2, 1.6 Hz, 1H), 7.02 (s, 2H). |
| 73 | 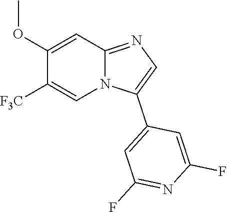 [SMILES: COc1cc2ncc(-c3cc(F)nc(F)c3)n2cc1C(F)(F)F] | 3-(2,6- difluoropyridin-4-yl)- 7-methoxy-6- (trifluoromethyl) imidazo[1,2-a]pyridine | [M + H]⁺ = 330.4 |
| 74 | 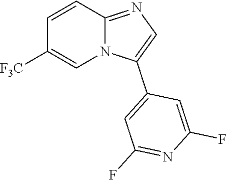 [SMILES: Fc1cc(-c2cnc3ccc(C(F)(F)F)cn23)cc(F)n1] | 3-(2,6- difluoropyridin-4-yl)- 6-(trifluoromethyl) imidazo[1,2-a]pyridine | [M + H]⁺ = 299.9 ¹H NMR (400 MHZ, CDCl₃) δ ppm 8.73 (s, 1H), 8.03 (s, 1H), 7.88 (d, J = 9.6 Hz, 1H), 7.50 (d, J = 9.2 Hz, 1H), 7.04 (s, 2H). |
| 75 | 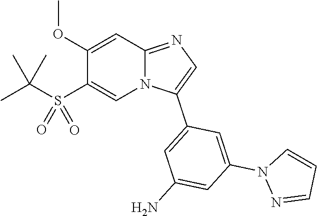 [SMILES: COc1cc2ncc(-c3cc(N)cc(-n4cccn4)c3)n2cc1S(=O)(=O)C(C)(C)C] | 3-(6-(tert- butylsulfonyl)-7- methoxyimidazo[1,2- a]pyridin-3-yl)-5- (1H-pyrazol-1- yl)aniline | [M + H]⁺ = 425.9 ¹H NMR (400 MHZ, DMSO-d₆) δ ppm 8.78 (s, 1H), 8.35 (d, J = 2.4 Hz, 1H), 7.75 (s, 1H), 7.72 (d, J = 1.6 Hz, 1H), 7.26 (s, 1H), 7.20-7.13 (m, 2H), 6.65 (s, 1H), 6.58-6.48 (m, 1H), 5.69 (s, 2H), 3.94 (s, 3H), 1.31 (s, 9H). |
| 76 | 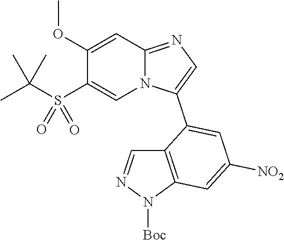 [SMILES: COc1cc2ncc(-c3cc([N+](=O)[O-])cc4c3cnn4C(=O)OC(C)(C)C)n2cc1S(=O)(=O)C(C)(C)C] | tert-butyl 4-(6-(tert- butylsulfonyl)-7- methoxyimidazo[1,2- a]pyridin-3-yl)-6- nitro-1H-indazole-1- carboxylate | [M − Boc + H]⁺ = 430.4 |
| 77 | 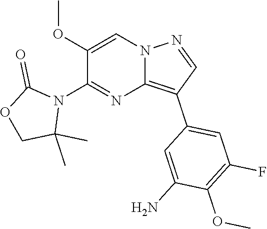 [SMILES: COc1cn2ncc(-c3cc(N)c(OC)c(F)c3)c2nc1N1C(=O)OCC1(C)C] | 3-(3-(3-amino-5- fluoro-4- methoxyphenyl)-6- methoxypyrazolo [1,5-a]pyrimidin-5-yl)- 4,4-dimethyloxazolidin- 2-one | ¹H NMR (400 MHZ, DMSO-d₆) δ ppm 9.07 (s, 1H), 8.47 (s, 1H), 7.15-7.05 (m, 2H), 5.26- 5.17 (m, 2H), 4.31 (s, 2H), 3.90 (s, 3H), 3.76 (s, 3H), 1.52 (s, 6H). |
| 78 | 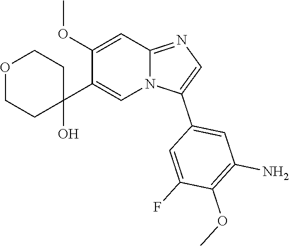 [SMILES: COc1cc2ncc(-c3cc(N)c(OC)c(F)c3)n2cc1C1(O)CCOCC1] | 4-(3-(3-amino-5- fluoro-4- methoxyphenyl)-7- methoxyimidazo[1,2- a]pyridin-6- yl)tetrahydro-2H- pyran-4-ol | ¹H NMR (400 MHZ, DMSO-d₆) δ ppm 8.57 (s, 1H), 7.46 (s, 1H), 7.02 (s, 1H), 6.69 (s, 1H), 6.66-6.60 (m, 1H), 5.43 (s, 2H), 5.29 (s, 1H), 3.91 (s, 3H), 3.79 (s, 3H), 3.78-3.73 (m, 2H), 3.72- 3.66 (m, 2H), 2.62-2.53 (m, 2H), 1.35-1.26 (m, 2H). |
| 79 | 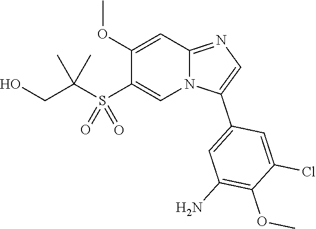 [SMILES: COc1cc2ncc(-c3cc(N)c(OC)c(Cl)c3)n2cc1S(=O)(=O)C(C)(C)CO] | 2-((3-(3-amino-5- chloro-4- methoxyphenyl)-7- methoxyimidazo[1,2- a]pyridin-6- yl)sulfonyl)-2- methylpropan-1-ol | ¹H NMR (400 MHZ, DMSO-d₆) δ ppm 8.70 (s, 1H), 7.64 (s, 1H), 7.20 (s, 1H), 6.83-6.77 (m, 2H), 5.58 (s, 2H), 5.01- 4.93 (m, 1H), 3.93 (s, 3H), 3.76 (s, 3H), 3.56 (d, J = 5.6 Hz, 2H), 1.28 (s, 6H). |
| 80 | 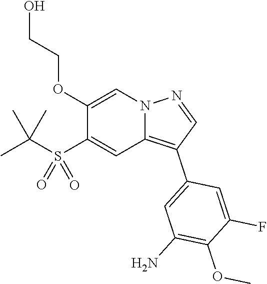 [SMILES: COc1c(N)cc(-c2cnn3cc(OCCO)c(S(=O)(=O)C(C)(C)C)cc23)cc1F] | 2-((3-(3-amino-5- fluoro-4- methoxyphenyl)-5- (tert-butylsulfonyl) pyrazolo[1,5-a]pyridin-6- yl)oxy)ethan-1-ol | [M + H]⁺ = 438.0 ¹H NMR (400 MHZ, DMSO-d₆) δ ppm 8.75 (s, 1H), 8.29 (s, 1H), 8.20 (s, 1H), 6.72 (s, 1H), 6.65-6.58 (m, 1H), 5.46 (s, 2H), 4.83 (t, J = 5.2 Hz, 1H), 4.15 (t, J = 5.2 Hz, 2H), 3.78 (s, 3H), 3.77-3.73 (m, 2H), 1.34 (s, 9H). |
| 81 | 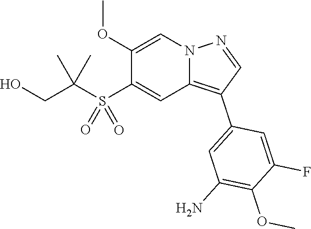 [SMILES: COc1cn2ncc(-c3cc(N)c(OC)c(F)c3)c2cc1S(=O)(=O)C(C)(C)CO] | 2-((3-(3-amino-5- fluoro-4- methoxyphenyl)-6- methoxypyrazolo [1,5-a]pyridin-5- yl)sulfonyl)-2- methylpropan-1-ol | [M + H]⁺ = 424.1 ¹H NMR (400 MHZ, DMSO-d₆) δ ppm 8.46 (s, 1H), 8.37 (s, 1H), 8.16 (s, 1H), 6.84 (s, 1H), 6.69 (dd, J = 12.0, 2.0 Hz, 1H), 3.99 (s, 3H), 3.93 (s, 3H), 3.76 (s, 2H), 1.41 (s, 6H). |
| 82 | 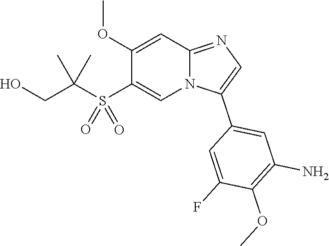 [SMILES: COc1cc2ncc(-c3cc(N)c(OC)c(F)c3)n2cc1S(=O)(=O)C(C)(C)CO] | 2-((3-(3-amino-5- fluoro-4- methoxyphenyl)-7- methoxyimidazo[1,2- a]pyridin-6- yl)sulfonyl)-2- methylpropan-1-ol | ¹H NMR (400 MHZ, DMSO-d₆) δ ppm 8.72 (s, 1H), 7.63 (s, 1H), 7.20 (s, 1H), 6.66 (s, 1H), 6.62 (dd, J = 11.6, 2.0 Hz, 1H), 5.53 (s, 2H), 3.92 (s, 3H), 3.81 (s, 3H), 3.56 (d, J = 5.6 Hz, 2H), 1.28 (s, 6H). |
| 83 | 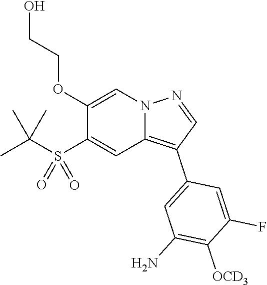 | 5-(6-(tert- butylsulfonyl)-7- methoxyimidazo[1,2- a]pyridin-3-yl)-3- fluoro-2-(methoxy- d3)aniline | [M + H]⁺ =411.0 ¹H NMR (400 MHZ, DMSO-d₆) δ ppm 8.69 (s, 1H), 7.64 (s, 1H), 7.25 (s, 1H), 6.65 (s, 1H), 6.62 (dd, J = 11.6, 2.0 Hz, 1H), 3.92 (s, 3H), 1.31 (s, 9H). |
| 84 | 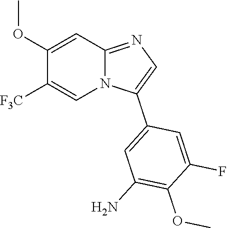 [SMILES: COc1cc2ncc(-c3cc(N)c(OC)c(F)c3)n2cc1C(F)(F)F] | 3-fluoro-2-methoxy- 5-(7-methoxy-6- (trifluoromethyl) imidazo[1,2-a]pyridin-3- yl)aniline | |
| 85 | 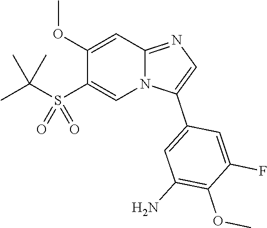 [SMILES: COc1cc2ncc(-c3cc(N)c(OC)c(F)c3)n2cc1S(=O)(=O)C(C)(C)C] | 5-(6-(tert- butylsulfonyl)-7- methoxyimidazo[1,2- a]pyridin-3-yl)-3- fluoro-2- methoxyaniline | [M + H]⁺ = 408.0 ¹H NMR (400 MHZ, DMSO-d₆) δ ppm 8.69 (s, 1H), 7.64 (s, 1H), 7.24 (s, 1H), 6.67-6.60 (m, 2H), 5.54 (s, 2H), 3.92 (s, 3H), 3.81 (s, 3H), 1.31 (s, 9H). |
| 86 | 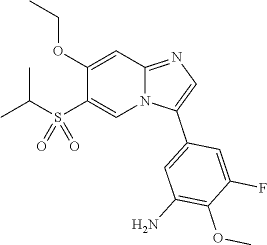 [SMILES: CCOc1cc2ncc(-c3cc(N)c(OC)c(F)c3)n2cc1S(=O)(=O)C(C)C] | 5-(7-ethoxy-6- (isopropylsulfonyl)i midazo[1,2- a]pyridin-3-yl)-3- fluoro-2- methoxyaniline | |

### Intermediate 87

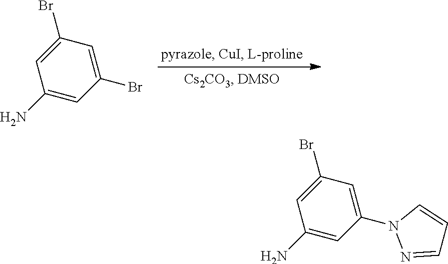 [SMILES: Nc1cc(Br)cc(Br)c1 ; Nc1cc(Br)cc(-n2cccn2)c1]

3-bromo-5-(1H-pyrazol-1-yl)aniline: To a solution of 3,5-dibromoaniline (1 g, 3.99 mmol, 1 eq) and pyrazole (244 mg, 3.59 mmol, 0.9 eq) in DMSO (20 mL) was added L-proline (83 mg, 717 μmol, 0.18 eq), Cs₂CO₃ (2.34 g, 7.17 mmol, 1.8 eq) and CuI (76 mg, 399 μmol, 0.1 eq). The mixture was stirred at 110° C. for 36 h under N₂ before it was diluted with H₂O (30 mL) and extracted with EtOAc (3×20 mL). The combined organic extracts were dried over Na₂SO₄, filtered, and concentrated under reduced pressure. The residue was purified by column chromatography (SiO₂, petroleum ether/EtOAc=10/1 to 3/1) to give the title compound (251 mg, 24% yield) as a black-brown solid. [M+H]⁺=238.1. ¹H NMR (400 MHz, DMSO-d₆) δ ppm 8.38 (d, J=2.4 Hz, 1H), 7.69 (s, 1H), 7.11 (t, J=1.6 Hz, 1H), 7.05 (t, J=1.6 Hz, 1H), 6.65 (t, J=1.6 Hz, 1H), 6.54-6.45 (m, 1H), 5.68 (s, 2H).

### Intermediate 88

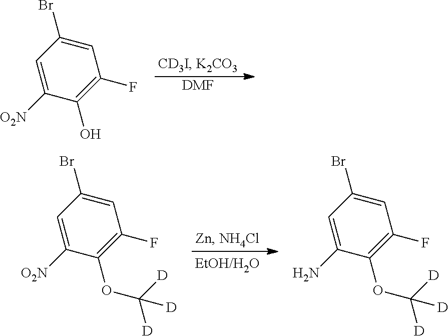

5-bromo-1-fluoro-2-(methoxy-d₃)-3-nitrobenzene: To a solution of 4-bromo-2-fluoro-6-nitrophenol (0.7 g, 2.97 mmol, 1 eq) in DMF (10 mL) was added CD₃I (631 mg, 4.45 mmol, 271 μL, 1.5 eq) and K₂CO₃ (1.23 g, 8.90 mmol, 3 eq). The mixture was stirred at 60° C. for 1 h before it was diluted with water (20 mL) and extracted with EtOAc (3×20 mL). The combined organic layers were dried over Na₂SO₄, filtered and concentrated under reduced pressure to give a residue, which was purified by column chromatography (SiO₂, 0-10% EtOAc/petroleum ether) to give the title compound (450 mg, 54% yield) as a light-yellow solid. ¹H NMR (400 MHz, CDCl₃) δ ppm 7.73 (t, J=2.0 Hz, 1H), 7.50 (dd, J=9.6, 2.4 Hz, 1H).

5-bromo-3-fluoro-2-(methoxy-d₃)aniline: To a solution of 5-bromo-1-fluoro-2-(methoxy-d₃)-3-nitrobenzene (450 mg, 1.60 mmol, 1 eq) in EtOH (20 mL) and H₂O (12 mL) were added Zn (1.05 g, 16.0 mmol, 10 eq) and NH₄Cl (856 mg, 16.0 mmol, 10 eq). The mixture was stirred at 80° C. for 10 min before it was cooled to 25° C. and stirred for 2 h. The reaction mixture was quenched with 1 N HCl (1 mL), and then diluted with water (20 mL) and extracted with DCM (3×20 mL). The combined organic layers were dried over Na₂SO₄, filtered, and concentrated under reduced pressure to give the title compound (347 mg, 87% yield) as a light yellow oil, which was used without purification. [M+H]⁺=224.9. ¹H NMR (400 MHz, CDCl₃) δ ppm 6.65-6.64 (m, 3H), 6.62 (d, J=2.4 Hz, 1H).

### Intermediate 89

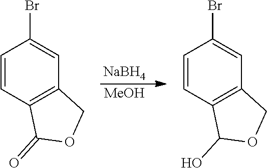 [SMILES: "MeOH" ; O=C1OCc2cc(Br)ccc21 ; "NaBH4" ; OC1OCc2cc(Br)ccc21]

5-bromo-1,3-dihydroisobenzofuran-1-ol: To a mixture of 5-bromoisobenzofuran-1 (3H)-one (100 mg, 469.42 μmol, 1 eq) in MeOH (5 mL) was added NaBH₄ (35.52 mg, 938.84 μmol, 2 eq) at 0° C. The mixture was degassed and purged with N₂ (3×), and then the mixture was stirred at 0° C. for 0.5 h under a N₂ atmosphere. The reaction mixture was subsequently quenched with saturated NH₄Cl (5 mL) and extracted with EtOAc (3×10 mL). The combined organic layers were dried over Na₂SO₄, filtered and concentrated in vacuo to yield the title compound, which was used for the next step without further purification. ¹H NMR (400 MHz, DMSO-d₆) δ ppm 7.30 (d, J=8.4 Hz, 1H), 7.09-7.05 (m, 2H), 5.67 (d, J=5.6 Hz, 1H), 5.25-5.15 (m, 1H), 4.59-4.49 (m, 1H), 4.30-4.22 (m, 1H).

### Intermediate 90

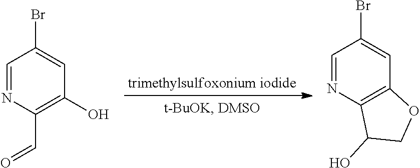 [SMILES: O=Cc1ncc(Br)cc1O ; OC1COc2cc(Br)cnc21]

6-bromo-2,3-dihydrofuro[3,2-b]pyridin-3-ol: To a solution of 5-bromo-3-hydroxypicolinaldehyde (200 mg, 990.07 μmol, 1 eq) in DMSO (10 mL) was added trimethylsulfoxonium iodide (544.72 mg, 2.48 mmol, 2.5 eq) and t-BuOK (277.75 mg, 2.48 mmol, 2.5 eq). The mixture was stirred at 20° C. for 80 min before it was quenched with saturated NH₄Cl (10 mL) at 0° C., and then diluted with H₂O (10 mL) and extracted with EtOAc (3×30 mL). The combined organic layers were washed with brine (3×30 mL), dried over Na₂SO₄, filtered and concentrated in vacuo. The resulting crude material was purified column chromatography (SiO₂, 0-30% EtOAc/PE) to yield the title compound. ¹H NMR (400 MHz, DMSO-d₆) δ ppm 8.22 (s, 1H), 7.62 (s, 1H), 5.96 (d, J=6.0 Hz, 1H), 5.17-5.12 (m, 1H), 4.8 (dd, J=10.4, 6.8 Hz, 1H), 4.35 (dd, J=10.4, 2.8 Hz, 1H).

### Intermediate 91

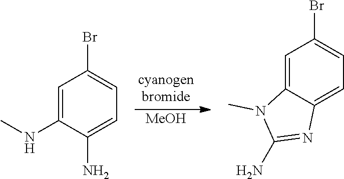

6-bromo-1-methyl-1H-benzo[d]imidazol-2-amine: To a solution of 5-bromo-N₁-methylbenzene-1,2-diamine (100 mg, 497.36 μmol, 1 eq) in MeOH (4 mL) was added cyanogen bromide (73.17 μL, 994.71 μmol, 2 eq). The mixture was stirred at 25° C. for 1 h before it was quenched with saturated NaHCO₃ (10 mL) at 25° C., diluted with H₂O (20 mL) and extracted with EtOAc (2×20 mL). The combined organic layers were washed with brine (20 mL), dried over Na₂SO₄, filtered and concentrated in vacuo to yield the title compound, which was used in the next step without further purification. LCMS [M+H]⁺=226.1. ¹H NMR (400 MHz, CDCl₃) δ ppm 7.24 (s, 3H), 3.55 (s, 3H).

### Intermediate 92

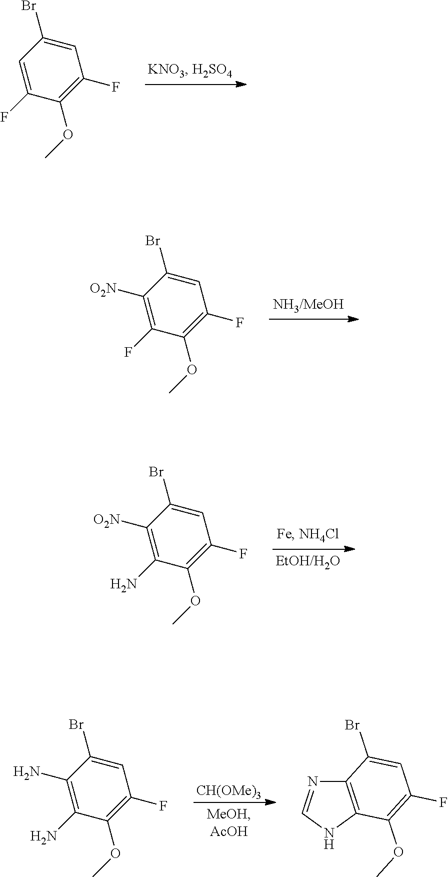

1-bromo-3,5-difluoro-4-methoxy-2-nitrobenzene: To a solution of 5-bromo-1,3-difluoro-2-methoxybenzene (2 g, 8.97 mmol, 1 eq) in H₂SO₄ (20 mL) was added KNO₃ (934 mg, 9.24 mmol, 1.03 eq) at 0° C. The mixture was stirred at 25° C. for 12 h before it was poured into ice/water (150 mL) and extracted with DCM (3×60 mL). The combined organic layers were washed with H₂O (150 mL), dried over Na₂SO₄, filtered and concentrated under reduced pressure to give a residue, which was purified by column chromatography (SiO₂, 0-5% EtOAc/petroleum ether) to give the title compound (2.15 g, 81% yield) as a light yellow oil. ¹H NMR (400 MHz, DMSO-d₆) δ ppm 7.96 (dd, J=10.8, 2.4 Hz, 1H), 4.04 (s, 3H).

3-bromo-5-fluoro-6-methoxy-2-nitroaniline: A mixture of 1-bromo-3,5-difluoro-4-methoxy-2-nitrobenzene (2 g, 6.72 mmol, 1 eq) in NH₃/MeOH (7 M, 25 mL) was stirred at 60° C. for 12 h. The reaction mixture was concentrated under reduced pressure to give a residue, which was purified by column chromatography (SiO₂, 0-5% EtOAc/petroleum ether) to give the title compound (1.4 g, 71% yield) as an orange solid. [M+H]⁺=264.8. ¹H NMR (400 MHz, DMSO-d₆) δ ppm 6.99 (d, J=10.4 Hz, 1H), 6.35 (s, 2H), 3.81 (s, 3H).

6-bromo-4-fluoro-3-methoxybenzene-1,2-diamine: A mixture of 3-bromo-5-fluoro-6-methoxy-2-nitroaniline (1.14 g, 3.87 mmol, 1 eq), Fe powder (2.16 g, 38.7 mmol, 10 eq) and NH₄Cl (2.07 g, 38.7 mmol, 10 eq) in EtOH (15 mL) and H₂O (1.5 mL) was degassed and purged with N₂ three times, and then the mixture was stirred at 80° C. for 12 h under N₂. The reaction mixture was filtered and concentrated under reduced pressure to give a residue. The residue was diluted with H₂O (30 mL) and extracted with EtOAc (3×30 mL). The combined organic layers were dried over Na₂SO₄, filtered, and concentrated under reduced pressure to give a residue, which was purified by column chromatography (SiO₂, 10% EtOAc/petroleum ether) to give the title compound (890 mg, 88% yield) as an orange solid. [M+H]⁺=234.9. ¹H NMR (400 MHz, DMSO-d₆) δ ppm 6.61 (d, J=10.4 Hz, 1H), 4.92 (s, 2H), 4.55 (s, 2H), 3.71 (s, 3H).

4-bromo-6-fluoro-7-methoxy-1H-benzo[d]imidazole: To a solution of 6-bromo-4-fluoro-3-methoxybenzene-1,2-diamine (100 mg, 383 μmol, 1 eq) in MeOH (1 mL) was added CH(OMe)₃ (61 mg, 574 μmol, 63 μL, 1.5 eq) and AcOH (0.2 mL). The mixture was stirred at 80° C. for 1 h before it was concentrated under reduced pressure. The mixture was diluted with saturated NaHCO₃ (20 mL) and extracted with EtOAc (3×20 mL). The combined organic layers were dried over Na₂SO₄, filtered, and concentrated under reduced pressure to give a residue, which was purified by column chromatography (SiO₂, 40-50% EtOAc/petroleum ether) to give the title compound (80 mg, 77% yield) as a white solid. [M+H]⁺=244.9. ¹H NMR (400 MHz, DMSO-d₆) δ ppm 13.30-12.91 (m, 1H), 8.29 (s, 1H), 7.43 (d, J=12.0 Hz, 1H), 4.36-4.02 (m, 3H).

### Intermediate 93

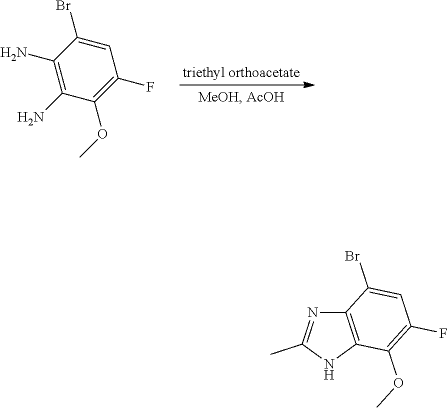

4-bromo-6-fluoro-7-methoxy-2-methyl-1H-benzo[d]imidazole: To a solution of 6-bromo-4-fluoro-3-methoxybenzene-1,2-diamine (200 mg, 766 μmol, 1 eq) in MeOH (2 mL) and AcOH (0.4 mL) was added triethyl orthoacetate (186 mg, 1.15 mmol, 210 μL, 1.5 eq). The mixture was stirred at 80° C. for 1 h before it was concentrated under reduced pressure. The mixture was diluted with saturated NaHCO₃ (20 mL) and extracted with EtOAc (3×15 mL). The combined organic layers were dried over Na₂SO₄, filtered, and concentrated under reduced pressure to give a residue, which was purified by column chromatography (SiO₂, petroleum ether/EtOAc=11/9 to 1/1) to give the title compound (200 mg, 91% yield) as a white solid. [M+H]⁺=258.8. ¹H NMR (400 MHz, CDCl₃) δ ppm 7.18 (d, J=12.0 Hz, 1H), 4.18 (d, J=1.6 Hz, 3H), 2.64 (s, 3H).

### Intermediate 94

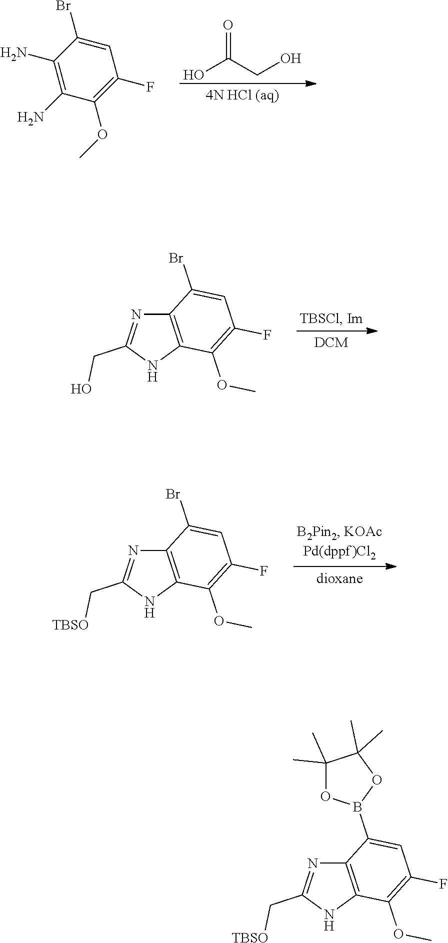 [SMILES: COc1c(F)cc(Br)c(N)c1N ; O=C(O)CO ; COc1c(F)cc(Br)c2nc(CO)[nH]c12 ; COc1c(F)cc(Br)c2nc(CO[Si](C)(C)C(C)(C)C)[nH]c12 ; COc1c(F)cc(B2OC(C)(C)C(C)(C)O2)c2nc(CO[Si](C)(C)C(C)(C)C)[nH]c12]

(4-bromo-6-fluoro-7-methoxy-1H-benzo[d]imidazol-2-yl)methanol: A mixture of 6-bromo-4-fluoro-3-methoxybenzene-1,2-diamine (500 mg, 1.91 mmol, 1 eq) and 2-hydroxyacetic acid (174.72 μL, 2.87 mmol, 1.5 eq) in 4 N HCl (6 mL) was stirred at 100° C. for 2 h. The reaction mixture was diluted with 10:1 DCM:MeOH (20 mL) and neutralized with saturated NaHCO₃. The resulting mixture was extracted with 10:1 DCM:MeOH (3×20 mL). The combined organic layers were dried over Na₂SO₄, filtered and concentrated in vacuo. The resulting crude material was purified by column chromatography (SiO₂, 5-7% MeOH/DCM) to yield the title compound. ¹H NMR (400 MHz, DMSO-d₆) δ ppm 13.19-12.63 (m, 1H), 7.37 (d, J=11.2 Hz, 1H), 5.68-5.48 (m, 1H), 4.64 (d, J=6.0 Hz, 2H), 4.25-3.95 (m, 3H).

4-bromo-2-(((tert-butyldimethylsilyl)oxy)methyl)-6-fluoro-7-methoxy-1H-benzo[d]imidazole: To a solution of (4-bromo-6-fluoro-7-methoxy-1H-benzo[d]imidazol-2-yl)methanol (210 mg, 687.09 μmol, 1 eq) in DCM (10 mL) was added TBSCl (253.61 μL, 2.06 mmol, 3 eq) and imidazole (140.33 mg, 2.06 mmol, 3 eq). The mixture was stirred at 25° C. for 2 h before it was diluted with H₂O (20 mL) and extracted with DCM (3×20 mL). The combined organic layers were dried over Na₂SO₄, filtered and concentrated in vacuo. The resulting crude material was purified by column chromatography (SiO₂, 5-10% EtOAc/PE) to yield the title compound. LCMS [M+H]⁺=390.9. ¹H NMR (400 MHz, DMSO-d₆) δ ppm 13.28-12.73 (m, 1H), 7.40 (d, J=11.6 Hz, 1H), 4.81 (s, 2H), 4.32-3.92 (m, 3H), 0.88 (s, 9H), 0.10 (s, 6H).

2-(((tert-butyldimethylsilyl)oxy)methyl)-6-fluoro-7-methoxy-4-(4,4,5,5-tetramethyl-1,3,2-dioxaborolan-2-yl)-1H-benzo[d]imidazole: A mixture of 4-bromo-2-(((tert-butyldimethylsilyl)oxy)methyl)-6-fluoro-7-methoxy-1H-benzo[d]imidazole (80 mg, 184.93 μmol, 1 eq), B₂Pin₂ (234.80 mg, 924.66 μmol, 5 eq), KOAc (54.45 mg, 554.79 μmol, 3 eq) and Pd(dppf)Cl₂ (13.53 mg, 18.49 μmol, 0.1 eq) in dioxane (1 mL) was degassed and purged with N₂ (3×). The reaction mixture was stirred at 100° C. for 12 h under a N₂ atmosphere before it was filtered and concentrated in vacuo to yield the title compound, which was used in the next step without further purification.

### Intermediate 95

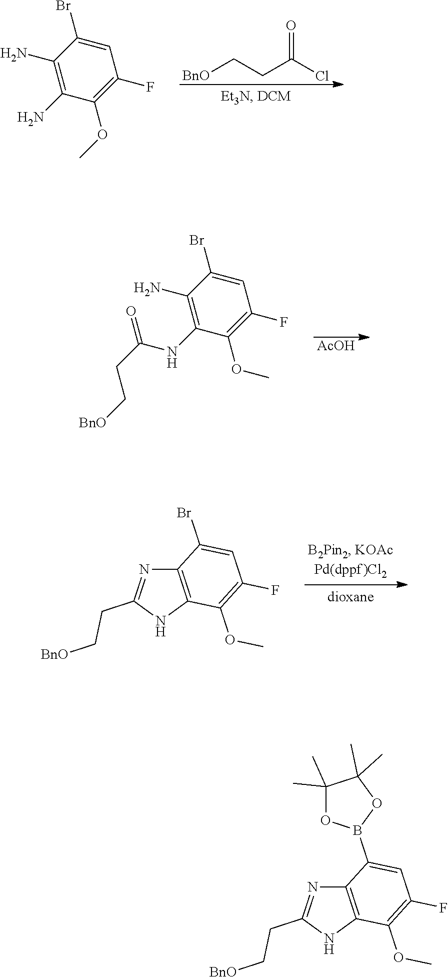

N-(2-amino-3-bromo-5-fluoro-6-methoxyphenyl)-3-(benzyloxy)propenamide: To a solution of 6-bromo-4-fluoro-3-methoxybenzene-1,2-diamine (200 mg, 765.78 μmol, 1 eq) and Et₃N (532.94 μL, 3.83 mmol, 5 eq) in DCM (4 mL) was added a solution of 3-(benzyloxy)propanoyl chloride (197.76 mg, 995.52 μmol, 1.3 eq) in DCM (1 mL) dropwise at 0° C. The mixture was stirred at 20° C. for 30 min before it was diluted with H₂O (30 mL) and extracted with EtOAc (3×30 mL). The combined organic layers were washed with brine (30 mL), dried over Na₂SO₄, filtered and concentrated in vacuo to yield the title compound, which was used in the next step without further purification.

2-(2-(benzyloxy)ethyl)-4-bromo-6-fluoro-7-methoxy-1H-benzo[d]imidazole: To N-(2-amino-3-bromo-5-fluoro-6-methoxyphenyl)-3-(benzyloxy)propenamide (200 mg, 503.48 μmol, 1 eq) was added AcOH (2 mL). The mixture was stirred at 80° C. for 1 h before it was neutralized with saturated NaHCO₃. The resulting mixture was diluted with H₂O (30 mL) and extracted with EtOAc (3×30 mL). The combined organic layers were washed with brine (30 mL), dried over Na₂SO₄, filtered and concentrated in vacuo. The resulting crude material was purified by column chromatography (SiO₂, 0-30% EtOAc/PE) to yield the title compound. LCMS [M+H]⁺=380.9. ¹H NMR (400 MHz, CDCl₃) δ ppm 7.31-7.22 (m, 5H), 7.09 (d, J=11.6 Hz, 1H), 4.51 (s, 2H), 4.05 (br s, 3H), 3.80 (t, J=5.6 Hz, 2H), 3.14 (t, J=5.6 Hz, 2H).

2-(2-(benzyloxy)ethyl)-6-fluoro-7-methoxy-4-(4,4,5,5-tetramethyl-1,3,2-dioxaborolan-2-yl)-1H-benzo[d]imidazole: A mixture of 2-(2-(benzyloxy)ethyl)-4-bromo-6-fluoro-7-methoxy-1H-benzo[d]imidazole (190 mg, 450.92 μmol, 1 eq), B₂Pin₂ (572.54 mg, 2.25 mmol, 5 eq), KOAc (132.76 mg, 1.35 mmol, 3 eq) and Pd(dppf)Cl₂ (32.99 mg, 45.09 μmol, 0.1 eq) in 1,4-dioxane (5 mL) was degassed and purged with N₂ (3×). The reaction mixture was stirred at 100° C. for 12 h under a N₂ atmosphere before it was filtered and concentrated in vacuo to yield the title compound, which was used in the next step without further purification.

### Intermediate 96

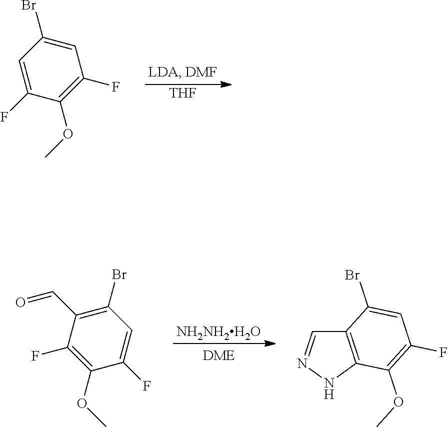

6-bromo-2,4-difluoro-3-methoxybenzaldehyde: To a solution of 5-bromo-1,3-difluoro-2-methoxybenzene (1 g, 4.48 mmol, 1 eq) in THF (5 mL) was added LDA (2 M, 2.8 mL, 1.24 eq) at −78° C. under N₂. The mixture was stirred at −60° C. for 1 h, and then DMF (475 mg, 6.50 mmol, 500 μL, 1.45 eq) was added in one portion. The resulting mixture was stirred at −60° C. for 1 h under N₂ before it was quenched with saturated NH₄Cl (50 mL) and then extracted with EtOAc (3×20 mL). The combined organic layers were washed with brine, dried over Na₂SO₄, filtered, and concentrated under reduced pressure to give the title compound (1.06 g) as a yellow solid, which was used without further purification. ¹H NMR (400 MHz, DMSO-d₆) δ ppm 10.10 (s, 1H), 7.81-7.75 (m, 1H), 3.96 (s, 3H).

4-bromo-6-fluoro-7-methoxy-1H-indazole: To a solution of 6-bromo-2,4-difluoro-3-methoxybenzaldehyde (960 mg, 3.82 mmol, 1 eq) in DME (4.8 mL) was added H₂NNH₂·H₂O (4.95 g, 84.1 mmol, 4.8 mL, 85% purity, 22 eq). The mixture was stirred at 90° C. for 3 h under N₂ before it was quenched with 10% HCl (50 mL) at 0° C., and then extracted with EtOAc (50 mL×3). The combined organic layers were washed with brine, dried over sodium sulfate, filtered, and concentrated to give a residue, which was purified by column chromatography (SiO₂, 0-30% EtOAc/petroleum ether) to give the title compound (470 mg, 45% yield) as a yellow solid. ¹H NMR (400 MHz, DMSO-d₆) δ ppm 13.99-13.65 (m, 1H), 8.05 (s, 1H), 7.41-7.35 (m, 1H), 4.05 (s, 3H).

### Intermediate 97

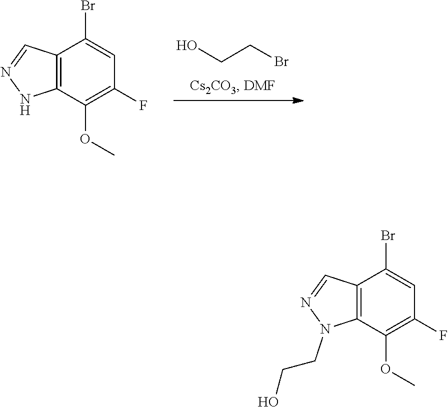

2-(4-bromo-6-fluoro-7-methoxy-1H-indazol-1-yl)ethan-1-ol: To a solution of 4-bromo-6-fluoro-7-methoxy-1H-indazole (250 mg, 920 μmol, 1 eq) in DMF (5 mL) was added Cs₂CO₃ (598 mg, 1.84 mmol, 2 eq) and 2-bromoethan-1-ol (230 mg, 1.84 mmol, 130 μL, 2 eq). The mixture was stirred at 80° C. for 2 h before it was diluted with water and extracted with EtOAc. The combined organic layers were washed with brine (30 mL), dried over Na₂SO₄, filtered, and concentrated under reduced pressure to give a residue, which was purified by column chromatography (SiO₂, 1-100% EtOAc/petroleum ether) to give the title compound (130 mg, 44% yield) as a yellow oil. [M+H]⁺=290.9). ¹H NMR (400 MHz, CDCl₃) δ ppm 7.94 (s, 1H), 7.12 (d, J=11.2 Hz, 1H), 4.75-4.69 (m, 2H), 4.52 (s, 1H), 4.15-4.06 (m, 5H).

### Intermediate 98

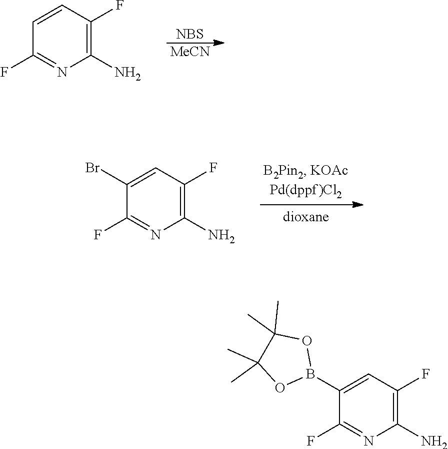

5-bromo-3,6-difluoropyridin-2-amine: To a mixture of 3,6-difluoropyridin-2-amine (130 mg, 999.27 μmol, 1 eq) in MeCN (15 mL) was added NBS (124.50 mg, 699.49 μmol, 0.7 eq). The mixture was stirred in absence of light at 25° C. for 30 min, and then a solution of additional NBS (124.50 mg, 699.49 μmol, 0.7 eq) in MeCN (5 mL) was added. The mixture was stirred in absence of light at 25° C. for another 12 hours under a N₂ atmosphere before it was concentrated under reduced pressure. The residue was purified by column chromatography (SiO₂, 0-30% EtOAc/petroleum ether) to give the title compound (180 mg, 78% yield) as a yellow solid. ¹H NMR (400 MHz, CDCl₃) δ ppm 7.58-7.34 (m, 1H), 4.98-4.37 (m, 2H).

3,6-difluoro-5-(4,4,5,5-tetramethyl-1,3,2-dioxaborolan-2-yl)pyridin-2-amine: To a mixture of 5-bromo-3,6-difluoropyridin-2-amine (140 mg, 602.90 μmol, 90% purity, 1 eq), B₂Pin₂ (459.29 mg, 1.81 mmol, 3 eq) and KOAc (177.50 mg, 1.81 mmol, 3 eq) in dioxane (10 mL) was added Pd(dppf)Cl₂ (44.11 mg, 60.29 μmol, 0.1 eq). The mixture was degassed and purged with N₂ 3 times, and then it was stirred at 100° C. for 12 hours under a N₂ atmosphere. The reaction mixture concentrated under reduced pressure to give a residue, which was purified by column chromatography (SiO₂, 0-30% EtOAc/petroleum ether) to give the title compound (90 mg, 52% yield) as yellow oil. ¹H NMR (400 MHz, CDCl₃) δ ppm 7.63-7.55 (m, 1H), 4.89 (s, 2H), 1.33 (s, 12H).

### Intermediate 99

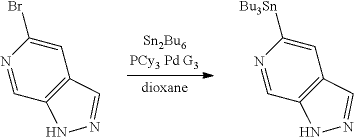

5-(tributylstannyl)-1H-pyrazolo[3,4-c]pyridine: A mixture of 5-bromo-1H-pyrazolo[3,4-c]pyridine (100 mg, 454.50 μmol, 1 eq), PCy₃ Pd G3 (33.41 mg, 45.45 μmol, 0.1 eq), Sn₂Bu₆ (3.95 g, 6.82 mmol, 3.41 mL, 15 eq) in 1,4-dioxane (10 mL) was stirred at 110° C. for 12 h under a N₂ atmosphere, and then the mixture was stirred at 130° C. for 6 h under a N₂ atmosphere. The reaction mixture was diluted with H₂O (20 mL) and extracted with EtOAc (3×20 mL), dried over Na₂SO₄, filtered and concentrated under reduced pressure to give a residue, which was purified by column chromatography (SiO₂, petroleum ether/EtOAc=1/0 to 5/1) to give the title compound (100 mg, 49% yield) as yellow oil. [M+H]⁺=410.0. ¹H NMR (400 MHz, DMSO-d₆) δ ppm 13.51 (s, 1H), 9.18 (s, 1H), 8.14 (s, 1H), 7.92-7.77 (m, 1H), 1.60-1.47 (m, 6H), 1.33-1.24 (m, 6H), 1.13-1.01 (m, 6H), 0.85-0.79 (m, 9H).

### Intermediate 100

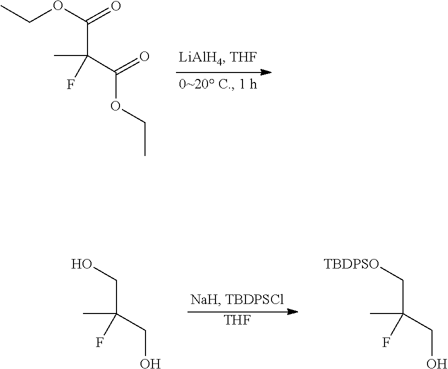

2-fluoro-2-methylpropane-1,3-diol: To a stirred solution of diethyl 2-fluoro-2-methylmalonate (1 g, 5.20 mmol, 1 eq) in THF (20 mL) was added dropwise LiAlH₄ (2.5 M, 5.20 mL, 2.5 eq) at 0° C. The reaction mixture was stirred at 20° C. for 2 h. After cooling to 0° C., the reaction was quenched by addition of water (0.5 mL), 15% NaOH (0.5 mL), and water (1 mL). The suspension was stirred for 30 min, after which solids were filtered and washed with THF (10 mL). The filtrate was evaporated to yield the title compound (400 mg, 2.96 mmol, 56.9% yield, 80% purity) as colorless oil. ¹H NMR: (400 MHz, CDCl₃) δ ppm: 3.78 (s, 2H), 3.73 (s, 2H), 2.28-2.26 (m, 2H), 1.44-1.30 (m, 3H).

3-((tert-butyldiphenylsilyl)oxy)-2-fluoro-2-methylpropan-1-ol: To a stirred solution of 2-fluoro-2-methylpropane-1,3-diol (320 mg, 2.37 mmol, 1 eq) in THF (10 mL) at 0° C. was added NaH (114 mg, 2.84 mmol, 60% purity, 1.2 eq). The mixture was stirred at 0° C. for 1 h, after which TBDPSCl (651 mg, 2.37 mmol, 606 μL, 1 eq) was added and the mixture was stirred at 0° C. for another 1 h. The reaction mixture was diluted with H₂O (10 mL) and extracted with EtOAc (20 mL×3). The combined organic layers were washed with brine (10 mL), dried over Na₂SO₄, filtered, and concentrated to give a residue, which was purified by flash silica gel chromatography (from PE/EtOAc=100/1 to 100/15) to yield the title compound (350 mg, 808 μmol, 34.1% yield, 80% purity) as colorless oil. ¹H NMR: (400 MHz, DMSO-d₆) δ ppm: 7.63 (dd, J=1.6, 7.6 Hz, 4H), 7.47-7.42 (m, 6H), 4.96 (t, J=6.0 Hz, 1H), 3.73 (s, 1H), 3.68 (d, J=2.8 Hz, 1H), 3.55-3.49 (m, 2H), 1.31-1.24 (m, 3H), 1.01 (s, 9H).

### Intermediate 101 (3-((tert-butyldiphenylsilyl)oxy)-2-fluoropropan-1-ol)

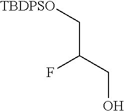

was prepared according to procedures analogous to those described for Intermediate 100. [M+Na]⁺=355.0. ¹H NMR: (400 MHz, DMSO-d₆) δ ppm: 7.63 (dd, J=1.6, 7.6 Hz, 4H), 7.51-7.40 (m, 6H), 4.92 (t, J=5.6 Hz, 1H), 4.68-4.48 (m, 1H), 3.91-3.74 (m, 2H), 3.64 (t, J=5.2 Hz, 1H), 3.58 (t, J=5.2 Hz, 1H), 1.00 (s, 9H).

### Intermediate 102

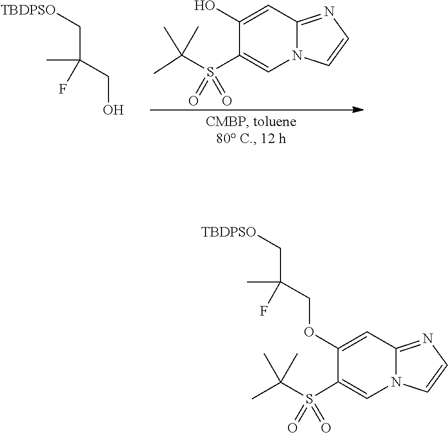

7-(3-((tert-butyldiphenylsilyl)oxy)-2-fluoro-2-methylpropoxy)-6-(tert-butylsulfonyl)imidazo[1,2-a]pyridine: To a stirred solution of 3-((tert-butyldiphenylsilyl)oxy)-2-fluoro-2-methylpropan-1-ol (307 mg, 708 μmol, 2.5 eq) and 6-(tert-butylsulfonyl)imidazo[1,2-a]pyridin-7-ol (80 mg, 283 μmol, 1 eq) in toluene (2 mL) was added CMBP (342 mg, 1.42 mmol, 5 eq). The reaction mixture was stirred at 80° C. for 12 h under N₂, after which it was concentrated under reduced pressure. The resulting residue was purified by flash silica gel chromatography (from PE/EtOAc=100/1 to 1/1) to yield the title compound (115 mg, 185 μmol, 65.5% yield, 94% purity) as yellow oil. [M+H]⁺=583.2. ¹H NMR: (400 MHz, DMSO-d₆) δ ppm: 9.14 (s, 1H), 8.00 (d, J=0.8 Hz, 1H), 7.64 (dd, J=1.6, 8.0 Hz, 4H), 7.55 (d, J=1.6 Hz, 1H), 7.46-7.39 (m, 6H), 7.17 (s, 1H), 4.36-4.22 (m, 2H), 4.07-4.04 (m, 1H), 3.94-3.86 (m, 1H), 1.53-1.23 (m, 3H), 1.24 (s, 9H), 1.01 (s, 9H).

The following compounds were prepared according to procedures analogous to those described for Intermediate 102.

| Int. | Structure | Name | LCMS, ¹H NMR |
|---|---|---|---|
| 103 | 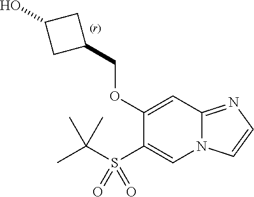 [SMILES: CC(C)(C)S(=O)(=O)c1cn2ccnc2cc1OC[C@H]1C[C@H](O)C1] | (1r,3r)-3-(((6-(tert- butylsulfonyl) imidazo[1,2-a]pyridin-7- yl)oxy)methyl) cyclobutan-1-ol | ¹H NMR: (400 MHZ, DMSO-d₆) δ ppm: 9.12 (s, 1H), 7.98 (s, 1H), 7.53 (d, J = 1.2 Hz, 1H), 7.15-7.05 (m, 1H), 5.02 (d, J = 6.0 Hz, 1H), 4.38-4.23 (m, 1H), 4.17-4.00 (m, 2H), 2.19-2.06 (m, 2H), 2.05-1.90 (m, 2H), 1.90-1.52 (m, 1H), 1.35 (s, 9H). |
| 104 | 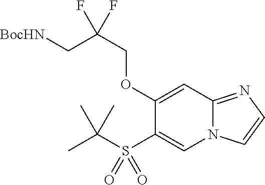 [SMILES: CC(C)(C)OC(=O)NCC(F)(F)COc1cc2nccn2cc1S(=O)(=O)C(C)(C)C] | tert-butyl (3-((6- (tert-butylsulfonyl) imidazo[1,2-a]pyridin-7- yl)oxy)-2,2- difluoropropyl)carbamate | ¹H NMR: (400 MHZ, DMSO-d₆) δ ppm: 9.17 (s, 1H), 8.03 (s, 1H), 7.58 (s, 1H), 7.28 (d, J = 1.2 Hz, 1H), 7.23 (s, 1H), 4.44 (t, J = 12.4 Hz, 2H), 3.72-3.61 (m, 2H), 1.38 (s, 9H), 1.31 (s, 9H). |
| 105 | 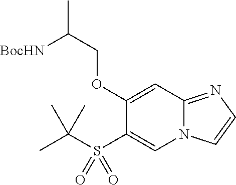 [SMILES: CC(COc1cc2nccn2cc1S(=O)(=O)C(C)(C)C)NC(=O)OC(C)(C)C] | tert-butyl (1-((6- (tert-butylsulfonyl) imidazo[1,2-a]pyridin-7- yl)oxy)propan-2- yl)carbamate | [M + H]⁺ = 412.2 |
| 106 | 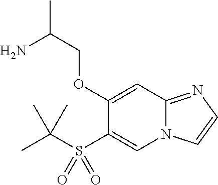 [SMILES: CC(N)COc1cc2nccn2cc1S(=O)(=O)C(C)(C)C] | 1-((6-(tert-butylsulfonyl) imidazo[1,2-a]pyridin-7- yl)oxy)propan-2- amine | ¹H NMR: (400 MHZ, DMSO-d₆) δ ppm: 9.16 (s, 1H), 8.00 (d, J = 1.2 Hz, 1H), 7.56 (d, J = 1.2 Hz, 1H), 7.18 (s, 1H), 4.13-4.07 (m, 1H), 3.98-3.90 (m, 1H), 2.57-2.53 (m, 1H), 1.33 (s, 9H), 1.18 (d, J = 6.8 Hz, 3H). |
| 107 | 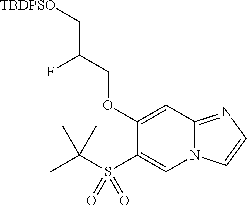 | 7-(3-((tert- butyldiphenylsilyl) oxy)-2- fluoropropoxy)-6- (tert-butylsulfonyl) imidazo[1,2-a]pyridine | [M + H]⁺ = 569.2 DMSO-d₆) δ ppm: 9.13 (s, 4.50-4.29 (m, 2H), 4.19-3.97 (s, 1H), 5.12-4.91 (m, 1H), (m, 4H), 7.55 (d, J = 1.2 Hz, ¹H NMR: (400 MHZ, (m, 2H), 1.25 (s, 9H), 1.02 1H), 7.51-7.37 (m, 6H), 7.15 1H), 8.00 (s, 1H), 7.71-7.61 (s, 9H). |

### Intermediate 108

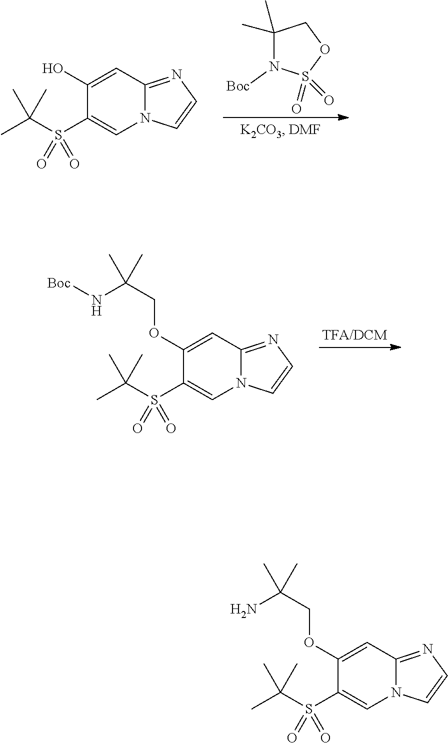 [SMILES: CC(C)(C)S(=O)(=O)c1cn2ccnc2cc1O ; CC(C)(C)OC(=O)N1C(C)(C)COS1(=O)=O ; CC(C)(COc1cc2nccn2cc1S(=O)(=O)C(C)(C)C)NC(=O)OC(C)(C)C ; CC(C)(N)COc1cc2nccn2cc1S(=O)(=O)C(C)(C)C]

tert-butyl (1-((6-(tert-butylsulfonyl)imidazo[1,2-a]pyridin-7-yl)oxy)-2-methylpropan-2-yl)carbamate: To 6-(tert-butylsulfonyl)imidazo[1,2-a]pyridin-7-ol (100 mg, 354 μmol, 1 eq) in DMF (2 mL) was added K₂CO₃ (97.8 mg, 708 μmol, 2 eq) and tert-butyl 4,4-dimethyl-1,2,3-oxathiazolidine-3-carboxylate 2,2-dioxide (107 mg, 425 μmol, 1.2 eq). The mixture was stirred at 60° C. for 12 h. The reaction mixture was concentrated under reduced pressure to give a residue, which was purified by flash silica gel chromatography (0-75% Ethyl acetate/Petroleum ether) to give the title compound (100 mg, 212 μmol, 59.8% yield, 90% purity) as a white solid. ¹H NMR: (400 MHz, DMSO-d₆) δ ppm: 9.13 (s, 1H), 7.99 (s, 1H), 7.55 (s, 1H), 7.10 (s, 1H), 6.49 (br s, 1H), 4.15 (br s, 2H), 1.36-1.32 (m, 24H).

1-((6-(tert-butylsulfonyl)imidazo[1,2-a]pyridin-7-yl)oxy)-2-methylpropan-2-amine: To a solution of tert-butyl (1-((6-(tert-butylsulfonyl)imidazo[1,2-a]pyridin-7-yl)oxy)-2-methylpropan-2-yl)carbamate (100 mg, 212 μmol, 1 eq) in DCM (0.6 mL) was added TFA (0.2 mL). The mixture was stirred at 25° C. for 30 min, after which it was concentrated to afford the title compound (68 mg, crude) as a yellow solid.

### Intermediate 109

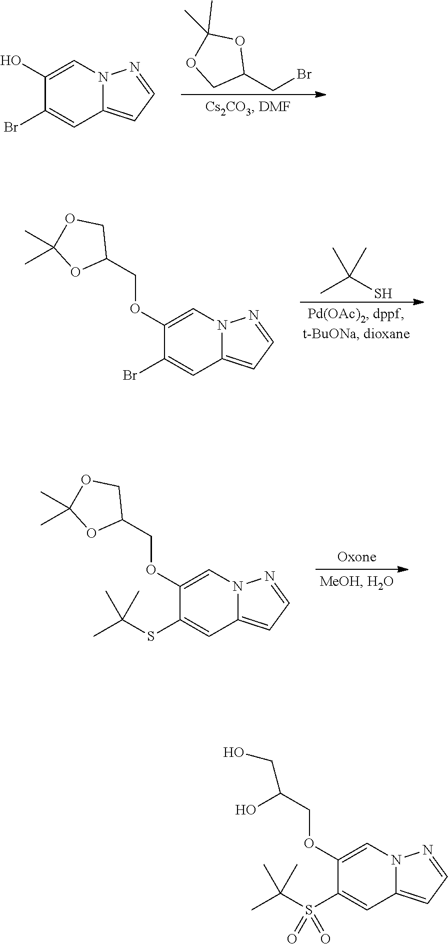

5-bromo-6-((2,2-dimethyl-1,3-dioxolan-4-yl)methoxy)pyrazolo[1,5-a]pyridine: To a solution of 5-bromopyrazolo[1,5-a]pyridin-6-ol (200 mg, 845 μmol, 1 eq) in DMF (5 mL) was added Cs₂CO₃ (826 mg, 2.53 mmol, 3 eq) and 4-(bromomethyl)-2,2-dimethyl-1,3-dioxolane (247 mg, 1.27 mmol, 1.5 eq). The mixture was stirred at 80° C. for 1 h. The reaction mixture was quenched by addition brine (20 mL) and extracted with EtOAc (50 mL). The combined organic layers were dried over Na₂SO₄, filtered, and concentrated under reduced pressure to give a residue, which was purified by column chromatography (SiO₂, Petroleum ether/Ethyl acetate=10/1 to 4/1) to give the title compound (135 mg, 371 μmol, 44% yield, 90% purity) as a yellow solid. [M+H]⁺=326.9. ¹H NMR: (400 MHz, DMSO-d₆) δ ppm: 8.59 (s, 1H), 8.09 (s, 1H), 7.92 (d, J=2.4 Hz, 1H), 6.53 (d, J=2.4 Hz, 1H), 4.48-4.43 (m, 1H), 4.19-4.10 (m, 3H), 3.88-3.80 (m 1H), 1.39 (s, 3H), 1.32 (s, 3H).

5-(tert-butylthio)-6-((2,2-dimethyl-1,3-dioxolan-4-yl)methoxy)pyrazolo[1,5-a]pyridine: A mixture of 5-bromo-6-((2,2-dimethyl-1,3-dioxolan-4-yl)methoxy)pyrazolo[1,5-a]pyridine (205 mg, 564 μmol, 1 eq), Pd(OAc)₂ (12.7 mg, 56.4 μmol, 0.1 eq), DPPF (62.5 mg, 113 μmol, 0.2 eq) and t-BuONa (163 mg, 1.69 mmol, 3 eq) in dioxane (3 mL) was degassed and purged with N₂ three times, and then 2-methylpropane-2-thiol (153 mg, 1.69 mmol, 190 μL, 3 eq) was added. The mixture was stirred at 90° C. for 12 h under N₂. The reaction mixture was filtered and concentrated under reduced pressure to give a residue, which was purified by column chromatography (SiO₂, Petroleum ether/Ethyl acetate=10/1 to 2/1) to give the title compound (188 mg, 503 μmol, 89.2% yield, 90% purity) as a light yellow solid. [M+H]⁺=337.0. ¹H NMR: (400 MHz, DMSO-d₆) δ ppm: 8.48 (s, 1H), 7.90 (d, J=2.0 Hz, 1H), 7.87 (s, 1H), 6.60 (d, J=2.0 Hz, 1H), 4.44-4.40 (m, 1H), 4.11-4.03 (m, 3H), 3.88-3.82 (m, 1H), 1.39 (s, 3H), 1.32 (s, 3H), 1.29 (s, 9H).

3-((5-(tert-butylsulfonyl)pyrazolo[1,5-a]pyridin-6-yl)oxy)propane-1,2-diol: To a solution of 5-(tert-butylthio)-6-((2,2-dimethyl-1,3-dioxolan-4-yl)methoxy)pyrazolo[1,5-a]pyridine (188 mg, 503 μmol, 1 eq) in MeOH (1.5 mL) and H₂O (0.3 mL) was added Oxone (464 mg, 754 μmol, 1.5 eq). The mixture was stirred at 25° C. for 12 h. The crude product (165 mg, crude) was used without further purification.

### Intermediate 110

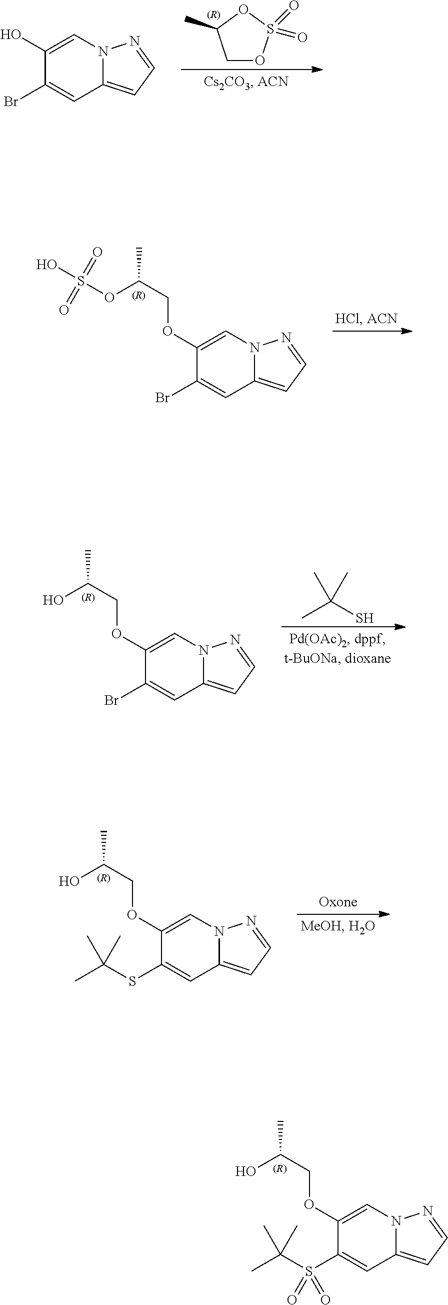

(R)-1-((5-bromopyrazolo[1,5-a]pyridin-6-yl)oxy)propan-2-yl hydrogen sulfate: To a solution of 5-bromopyrazolo[1,5-a]pyridin-6-ol (500 mg, 2.11 mmol, 1 eq) in ACN (7 mL) was added Cs₂CO₃ (2.06 g, 6.34 mmol, 3 eq) and (R)-4-methyl-1,3,2-dioxathiolane 2,2-dioxide (1.17 g, 8.45 mmol, 4 eq). The mixture was stirred at 25° C. for 12 h. The mixture was filtered and concentrated under reduced pressure to give the title compound (500 mg, crude) as dark brown oil that was used without further purification.

(R)-1-((5-bromopyrazolo[1,5-a]pyridin-6-yl)oxy)propan-2-ol: To a solution of (R)-1-((5-bromopyrazolo[1,5-a]pyridin-6-yl)oxy)propan-2-yl hydrogen sulfate (500 mg, 1.42 mmol, 1 eq) in ACN (1 mL) was added HCl (14.4 mg, 142 μmol, 14.1 μL, 36% purity, 0.1 eq) dropwise. The mixture was stirred at 50° C. for 1 h. The reaction mixture was concentrated under reduced pressure to remove ACN. The residue was purified by column chromatography (SiO₂, DCM/MeOH=100/0 to 88/12) to give the title compound (500 mg, crude) as a brown oil. ¹H NMR (400 MHz, DMSO-d₆) δ ppm: 8.53 (s, 1H), 8.08 (s, 1H), 7.91 (d, J=2.0 Hz, 1H), 6.52 (d, J=2.4 Hz, 1H), 4.93 (d, J=4.4 Hz, 1H), 4.03-3.94 (m, 2H), 3.90-3.85 (m, 1H), 1.20 (d, J=6.0 Hz, 3H).

(R)-1-((5-(tert-butylthio)pyrazolo[1,5-a]pyridin-6-yl)oxy)propan-2-ol: A mixture of (R)-1-((5-bromopyrazolo[1,5-a]pyridin-6-yl)oxy)propan-2-ol (500 mg, 1.84 mmol, 1 eq), 2-methylpropane-2-thiol (500 mg, 5.53 mmol, 623 μL, 3 eq), Pd(OAc)₂ (41.4 mg, 184 μmol, 0.1 eq), t-BuONa (532 mg, 5.53 mmol, 3 eq) and DPPF (204 mg, 369 μmol, 0.2 eq) in 1,4-dioxane (10 mL) was degassed and purged with N₂ three times, and then the mixture was stirred at 90° C. for 12 h under N₂. The reaction mixture was concentrated under reduced pressure to remove 1,4-dioxane. The residue was purified by column chromatography (SiO₂, DCM/MeOH=100/0 to 95/5) to give the title compound (430 mg, 1.38 mmol, 75% yield, 90% purity) as a brown oil. ¹H NMR (400 MHz, DMSO-d₆) δ ppm: 8.41 (s, 1H), 7.89 (s, 1H), 7.85 (s, 1H), 6.59 (s, 1H), 4.87 (d, J=4.0 Hz, 1H), 4.03-3.98 (m, 1H), 3.92-3.88 (m, 1H), 3.83-3.80 (m, 1H), 1.29 (s, 9H), 1.21 (d, J=6.0 Hz, 3H).

(R)-1-((5-(tert-butylsulfonyl)pyrazolo[1,5-a]pyridin-6-yl)oxy)propan-2-ol: To a solution of (R)-1-((5-(tert-butylthio)pyrazolo[1,5-a]pyridin-6-yl)oxy)propan-2-ol (430 mg, 1.38 mmol, 1 eq) in MeOH (9 mL) and H₂O (3 mL) was added Oxone (2.55 g, 4.14 mmol, 3 eq). The mixture was stirred at 25° C. for 2 h., after which it was concentrated to afford the title compound (430 mg, crude) as a brown liquid that was used without purification. [M+H]⁺=313.1.

### Intermediate 111

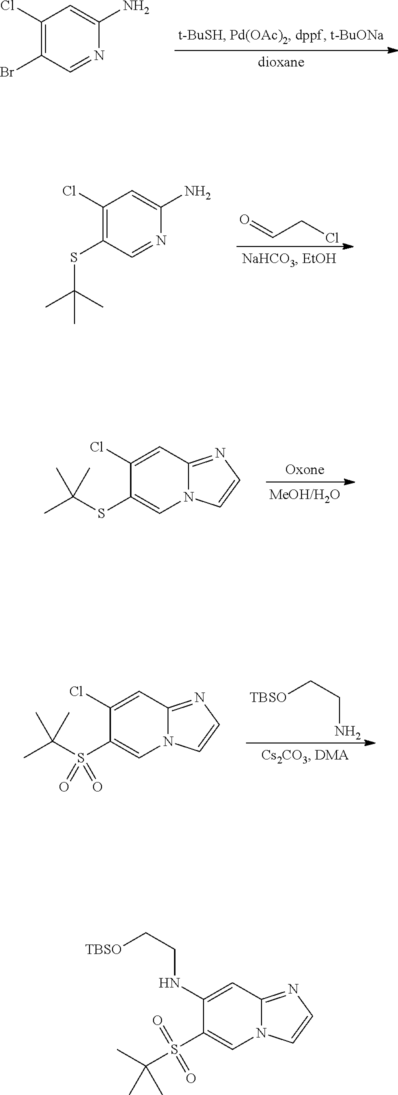

5-(tert-butylthio)-4-chloropyridin-2-amine: A mixture of 5-bromo-4-chloropyridin-2-amine (5 g, 24.1 mmol, 1 eq), 2-methylpropane-2-thiol (6.52 g, 72.3 mmol, 8.14 mL, 3 eq), DPPF (2.67 g, 4.82 mmol, 0.2 eq), Pd(OAc)₂ (541 mg, 2.41 mmol, 0.1 eq) and t-BuONa (6.95 g, 72.3 mmol, 3 eq) in 1,4-dioxane (50 mL) was degassed and purged with N₂ three times, and then the mixture was stirred at 90° C. for 12 h under N₂. The reaction mixture was concentrated under reduced pressure to give a residue, which was purified by column chromatography to give the title compound (5 g, 20.8 mmol, 97% yield, 90% purity) as a light yellow solid. [M+H]⁺=217.0.

¹H NMR: (400 MHz, CDCl₃) δ ppm: 8.22 (s, 1H), 6.63 (s, 1H), 4.63 (br s, 2H), 1.29 (s, 9H).

6-(tert-butylthio)-7-chloroimidazo[1,2-a]pyridine: To a solution of 5-(tert-butylthio)-4-chloropyridin-2-amine (5 g, 20.8 mmol, 1 eq) in EtOH (30 mL) was added NaHCO₃ (5.23 g, 62.3 mmol, 2.42 mL, 3 eq) and 2-chloroacetaldehyde (12.2 g, 62.3 mmol, 10.0 mL, 3 eq). The mixture was stirred at 80° C. for 12 h, after which it was filtered and concentrated under reduced pressure to remove EtOH. The residue was diluted with H₂O (100 mL) and extracted with EtOAc (3×). The combined organic layers were washed with brine (100 mL), dried over Na₂SO₄, filtered, and concentrated under reduced pressure to give a residue, which was purified by column chromatography (SiO₂, Petroleum ether/Ethyl acetate=100/0 to 1/1) to give the title compound (4 g, 15.0 mmol, 72.0% yield, 90% purity) as an off-white solid. ¹H NMR: (400 MHz, DMSO-d₆) δ ppm: 8.98 (s, 1H), 8.01 (s, 1H), 7.88 (s, 1H), 7.64 (s, 1H), 1.29 (s, 9H).

6-(tert-butylsulfonyl)-7-chloroimidazo[1,2-a]pyridine: To a solution of 6-(tert-butylthio)-7-chloroimidazo[1,2-a]pyridine (1 g, 3.74 mmol, 1 eq) in MeOH (15 mL) and H₂O (5 mL) was added Oxone (3.45 g, 5.61 mmol, 1.5 eq) at 0° C. The mixture was stirred at 25° C. 12 h. The reaction mixture was quenched by addition saturated Na₂SO₃ (200 mL) at 0° C. and extracted with EtOAc (100 mL×3). The combined organic layers were washed with brine (100 mL), dried over Na₂SO₄, filtered, and concentrated under reduced pressure to give the title compound (998 mg, 3.29 mmol, 88.1% yield, 90% purity) as a light-yellow solid. ¹H NMR: (400 MHz, DMSO-d₆) δ ppm: 9.41 (s, 1H), 8.22 (s, 1H), 7.94 (s, 1H), 7.77 (d, J=1.2 Hz, 1H), 1.36 (s, 9H).

N-(2-((tert-butyldimethylsilyl)oxy)ethyl)-6-(tert-butylsulfonyl)imidazo[1,2-a]pyridin-7-amine: To a solution of 6-(tert-butylsulfonyl)-7-chloroimidazo[1,2-a]pyridine (200 mg, 660 μmol, 1 eq) in DMA (3 mL) was added Cs₂CO₃ (645 mg, 1.98 mmol, 3 eq) and 2-((tert-butyldimethylsilyl)oxy)ethan-1-amine (347 mg, 1.98 mmol, 3 eq). The mixture was stirred at 80° C. for 2 h. The reaction mixture was diluted with H₂O (30 mL) and extracted with EtOAc (30 mL×3). The combined organic layers were washed with brine (30 mL), dried over Na₂SO₄, filtered, and concentrated under reduced pressure to give a residue, which was purified by column chromatography (SiO₂, Petroleum ether/Ethyl acetate=1/1 to 1/9) to give the title compound (110 mg, 241 μmol, 36.4% yield, 90% purity) as a light-yellow solid. [M+H]⁺=412.1. ¹H NMR: (400 MHz, DMSO-d₆) δ ppm: 8.98 (s, 1H), 7.79 (s, 1H), 7.38 (d, J=1.2 Hz, 1H), 6.55-6.47 (m, 2H), 3.83 (t, J=5.2 Hz, 2H), 3.24-3.22 (m, 2H), 1.32 (s, 9H), 0.89 (s, 9H), 0.08 (s, 6H).

### Intermediate 112

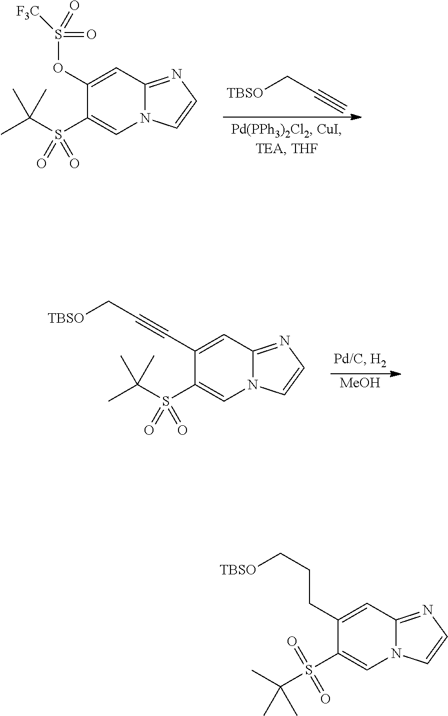

7-(3-((tert-butyldimethylsilyl)oxy)prop-1-yn-1-yl)-6-(tert-butylsulfonyl)imidazo[1,2-a]pyridine: A mixture of tert-butyldimethyl(prop-2-yn-1-yloxy)silane (159 mg, 932 μmol, 189 μL, 2 eq), 6-(tert-butylsulfonyl)imidazo[1,2-a]pyridin-7-yl trifluoromethanesulfonate (200 mg, 466 μmol, 1 eq), Pd(PPh₃)₂Cl₂ (65.4 mg, 93.2 μmol, 0.2 eq), CuI (8.87 mg, 46.6 μmol, 0.1 eq) and TEA (212 mg, 2.10 mmol, 292 μL, 4.5 eq) in THF (5 mL) was degassed and purged with N₂ three times, and then the mixture was stirred at 20° C. for 2 h under N₂. The mixture was filtered and concentrated under reduced pressure to give a residue, which was purified by column chromatography (SiO₂, Petroleum ether/Ethyl acetate=5/1 to 3/1) to give the title compound (150 mg, 332 μmol, 71.3% yield, 90% purity) as a brown solid. [M+H]⁺=407.1. ¹H NMR (400 MHz, DMSO-d₆) δ ppm: 9.32 (s, 1H), 8.36-8.17 (m, 1H), 8.00-7.70 (m, 2H), 4.58 (s, 2H), 1.35 (s, 9H), 0.90 (s, 9H), 0.14 (s, 6H).

7-(3-((tert-butyldimethylsilyl)oxy)propyl)-6-(tert-butylsulfonyl)imidazo[1,2-a]pyridine: To a solution of 7-(3-((tert-butyldimethylsilyl)oxy)prop-1-yn-1-yl)-6-(tert-butylsulfonyl)imidazo[1,2-a]pyridine (150 mg, 332 μmol, 1 eq) in MeOH (3 mL) was added Pd/C (80 mg, 75.2 μmol, 10% purity, 0.23 eq) under N₂. The suspension was degassed and purged with H₂ three times. The mixture was stirred under H₂ (15 Psi) at 25° C. for 12 h. The mixture was filtered and concentrated under reduced pressure to give the title compound (130 mg, crude) as a brown solid, which was used without further purification. [M+H]⁺=411.6.

### Intermediate 113

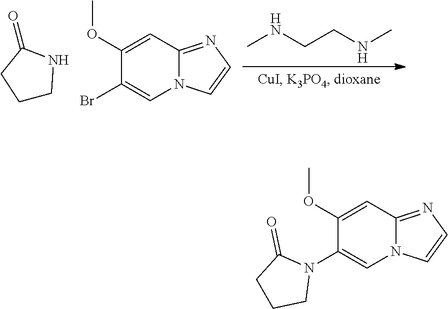

1-(7-methoxyimidazo[1,2-a]pyridin-6-yl)pyrrolidin-2-one: A mixture of 6-bromo-7-methoxyimidazo[1,2-a]pyridine (100 mg, 396 μmol, 1 eq), pyrrolidin-2-one (50.6 mg, 595 μmol, 45.6 μL, 1.5 eq), CuI (30.2 mg, 158 μmol, 0.4 eq), K₃PO₄ (252 mg, 1.19 mmol, 3 eq) and N¹,N²-dimethylethane-1,2-diamine (28.0 mg, 317 μmol, 34.1 L, 0.8 eq) in 1,4-dioxane (2 mL) was degassed and purged with N₂ three times, and then the mixture was stirred at 120° C. for 4 h under N₂. The reaction mixture was partitioned between DCM/MeOH=10/1 (20 mL) and H₂O (5 mL). The organic phase was separated, washed with brine (10 mL×3), dried over Na₂SO₄, filtered, and concentrated under reduced pressure to give a residue, which was purified by flash silica gel chromatography (0˜5% methanol/dichloromethane) to give the title compound (40 mg, 156 μmol, 39.3% yield, 90% purity) as a yellow oil. ¹H NMR: (400 MHz, CDCl₃) δ ppm: 8.06 (s, 1H), 7.50 (s, 1H), 7.40 (s, 1H), 6.96 (s, 1H), 3.89 (s, 3H), 3.76 (t, J=7.2 Hz, 2H), 2.58 (t, J=8.0 Hz, 2H), 2.26-2.17 (m, 2H).

The following compounds were prepared according to procedures analogous to those described for Intermediate 113.

| Int. | Structure | Name | LCMS, ¹H NMR |
|---|---|---|---|
| 114 | 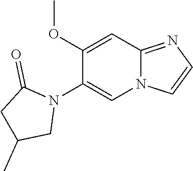 [SMILES: COc1cc2nccn2cc1N1CC(C)CC1=O] | 1-(7- methoxyimidazo[1,2- a]pyridin-6-yl)-4- methylpyrrolidin-2- one | [M + H]⁺ = 246.1 |
| 115 | 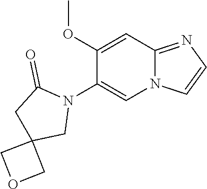 [SMILES: COc1cc2nccn2cc1N1CC2(COC2)CC1=O] | 6-(7- methoxyimidazo[1,2- a]pyridin-6-yl)-2-oxa- 6-azaspiro[3.4]octan- 7-one | ¹H NMR: (400 MHZ, CDCl₃) δ ppm: 8.24-7.95 (m, 1H), 7.65-7.31 (m, 2H), 7.22-7.02 (m, 1H), 4.80-4.76 (m, 2H), 4.74- 4.70 (m, 2H), 4.04 (s, 2H), 3.91 (s, 3H), 2.89 (s, 2H). |

### Intermediate 116

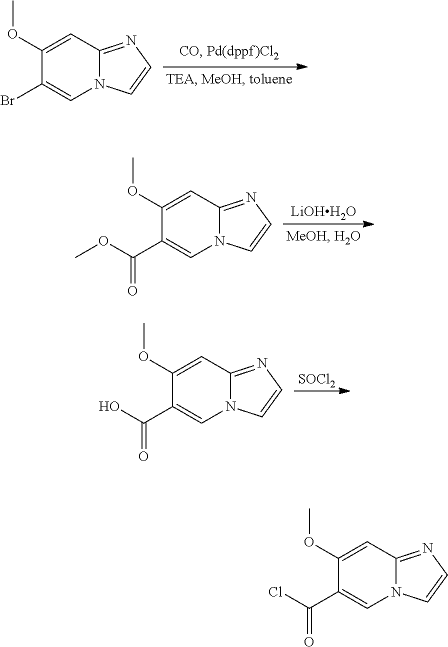

### methyl 7-methoxyimidazo[1,2-a]pyridine-6-carboxylate

To a mixture of 6-bromo-7-methoxyimidazo[1,2-a]pyridine (5 g, 19.8 mmol, 1 eq) in MeOH (50 mL) and toluene (50 mL) was added TEA (3.0 g, 29.7 mmol, 4.14 mL, 1.5 eq) and Pd(dppf)Cl₂ (1.45 g, 1.98 mmol, 0.1 eq). The mixture was stirred at 80° C. under a CO (40 Psi) atmosphere for 24 h. The reaction mixture was concentrated under reduced pressure to remove solvent. The residue was purified by flash silica gel chromatography (from EtOAc/MeOH=100/0 to 99/1) to yield methyl 7-methoxyimidazo[1,2-a]pyridine-6-carboxylate (4 g, 17.4 mmol, 88% yield) as a brown solid. [M+H]⁺=207.1. ¹H NMR: (400 MHz, CDCl₃) δ ppm: 8.71 (s, 1H), 7.57 (s, 1H), 7.48 (s, 1H), 6.93 (s, 1H), 3.94 (s, 3H), 3.92 (s, 3H).

### 7-methoxyimidazo[1,2-a]pyridine-6-carboxylic acid

To a solution of methyl 7-methoxyimidazo[1,2-a]pyridine-6-carboxylate (1 g, 4.36 mmol, 1 eq) in MeOH (20 mL) was added a solution of LiOH·H₂O (732 mg, 17.5 mmol, 4 eq) in H₂O (5 mL). The mixture was stirred at 25° C. for 12 h. The mixture was filtered, and the filtrate was concentrated to give the crude product, which was purified by prep-HPLC (100% H₂O) to give the title compound (600 mg, 2.81 mmol, 64% yield) as a yellow solid. ¹H NMR: (400 MHz, DMSO-d₆) δ ppm: 8.39 (s, 1H), 7.69 (s, 1H), 7.32 (d, J=1.2 Hz, 1H), 6.79 (s, 1H), 3.77 (s, 3H).

### 7-methoxyimidazo[1,2-a]pyridine-6-carbonyl chloride

To 7-methoxyimidazo[1,2-a]pyridine-6-carboxylic acid

(200 mg, 937 μmol, 1 eq) was added SOCl₂ (8 mL), and the resulting mixture was stirred at 60° C. for 2 h. The reaction mixture was concentrated under reduced pressure to remove solvent and give the title compound (600 mg) as a yellow solid, which was used without further purification.

### Intermediate 117

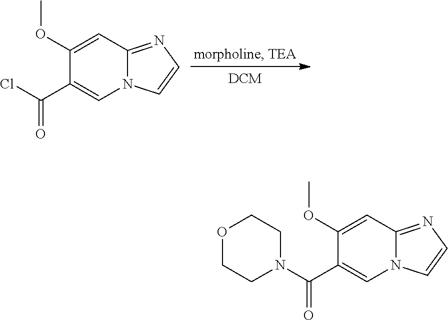 [SMILES: COc1cc2nccn2cc1C(=O)Cl ; COc1cc2nccn2cc1C(=O)N1CCOCC1]

(7-methoxyimidazo[1,2-a]pyridin-6-yl)(morpholino)methanone: To a solution of morpholine (124 mg, 1.42 mmol, 125 μL, 1.5 eq) in DCM (10 mL) was added TEA (288 mg, 2.85 mmol, 397 μL, 3 eq) and 7-methoxyimidazo[1,2-a]pyridine-6-carbonyl chloride (200 mg, 950 μmol, 1 eq) at 0° C. The mixture was stirred at 0° C. for 2 h. The mixture was evaporated under reduced pressure to give the title compound, which was used without further purification. [M+H]⁺=261.9.

The following compounds were prepared according to procedures analogous to that described for Intermediate 117.

| Int. | Structure | Name | LCMS, ¹H NMR |
|---|---|---|---|
| 118 | 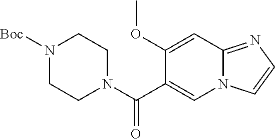 [SMILES: COc1cc2nccn2cc1C(=O)N1CCN(C(=O)OC(C)(C)C)CC1] | tert-butyl 4-(7- methoxyimidazo[1,2- a]pyridine-6- carbonyl)piperazine-1- carboxylate | [M + H]⁺ = 361.4 |
| 119 | 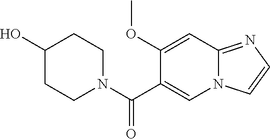 [SMILES: COc1cc2nccn2cc1C(=O)N1CCC(O)CC1] | (4-hydroxypiperidin-1- yl)(7- methoxyimidazo[1,2- a]pyridin-6- yl)methanone | [M + H]⁺ = 275.9 |
| 120 | 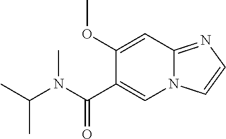 [SMILES: COc1cc2nccn2cc1C(=O)N(C)C(C)C] | N-isopropyl-7-methoxy- N-methylimidazo[1,2- a]pyridine-6- carboxamide | ¹H NMR: (400 MHZ, DMSO-d₆) δ ppm: 8.20 (dd, J = 5.2, 12.0 Hz, 2H), 7.76 (s, 1H), 7.19 (s, 1H), 3.96-3.92 (m, 3H), 3.73-3.63 (m, 1H), 2.88-2.84 (m, 3H), 1.18 (d, J = 6.4 Hz, 6H). |

### Intermediate 121

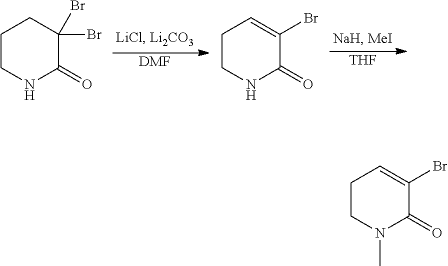

3-bromo-5,6-dihydropyridin-2 (1H)-one: To a solution of 3,3-dibromopiperidin-2-one (1 g, 3.89 mmol, 1 eq) in DMF (10 mL) was added LiCl (165 mg, 3.89 mmol, 1.0 eq) and Li₂CO₃ (431 mg, 5.84 mmol, 1.5 eq). The mixture was stirred at 100° C. for 12 h. The reaction mixture was filtered and concentrated under reduced pressure to give a residue, which was by column chromatography (SiO₂, Petroleum ether/Ethyl acetate=1/0 to 1/2) to give the title compound (500 mg, 2.56 mmol, 65.7% yield, 90% purity) as a yellow oil. ¹H NMR (400 MHz, CDCl₃) δ ppm: 7.05 (t, J=4.8 Hz, 1H), 6.50-6.09 (m, 1H), 3.58-3.35 (m, 2H), 2.52-2.31 (m, 2H).

3-bromo-1-methyl-5,6-dihydropyridin-2 (1H)-one: To a solution of (500 mg, 2.56 mmol, 1 eq) in THF (10 mL) was added NaH (409 mg, 10.2 mmol, 60% purity, 4.0 eq) at 0° C., the mixture was stirred at 0° C. for 10 min, then Mel (726 mg, 5.11 mmol, 318 μL, 2.0 eq) was added to the mixture. The mixture was stirred at 25° C. for 2 h under N₂ atmosphere. The reaction mixture was quenched by addition of saturated NH₄Cl (5 mL) at 0° C., after which it was concentrated. The resulting residue was purified by column chromatography (SiO₂, Petroleum ether/Ethyl acetate=1/0 to 1/2) to give the title compound (200 mg, 947 μmol, 37.1% yield, 90% purity) as a yellow oil. ¹H NMR: (400 MHz, CDCl₃) δ ppm: 6.93 (t, J=4.8 Hz, 1H), 3.46 (t, J=7.2 Hz, 2H), 3.05 (s, 3H), 2.49-2.33 (m, 2H).

### Intermediate 122

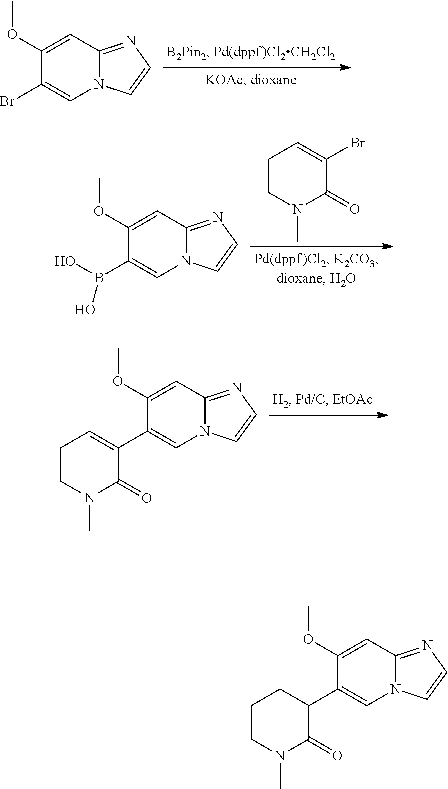

(7-methoxyimidazo[1,2-a]pyridin-6-yl)boronic acid: A mixture of 6-bromo-7-methoxyimidazo[1,2-a]pyridine (500 mg, 1.98 mmol, 1 eq), B₂Pin₂ (2.52 g, 9.91 mmol, 5.0 eq), KOAc (584 mg, 5.95 mmol, 3.0 eq), Pd(dppf)Cl₂·CH₂Cl₂ (162 mg, 199 μmol, 0.1 eq) in 1,4-dioxane (10 mL) was degassed and purged with N₂ three times, and then the mixture was stirred at 100° C. for 12 h under N₂. The reaction mixture was filtered and concentrated under reduced pressure to give the title compound (300 mg, crude) as dark brown liquid, which was used without further purification.

3-(7-methoxyimidazo[1,2-a]pyridin-6-yl)-1-methyl-5,6-dihydropyridin-2 (1H)-one: A mixture of (7-methoxyimidazo[1,2-a]pyridin-6-yl)boronic acid (300 mg, 1.56 mmol, 1 eq), 3-bromo-1-methyl-5,6-dihydropyridin-2 (1H)-one (330 mg, 1.56 mmol, 1.0 eq), Pd(dppf)Cl₂ (114 mg, 156 μmol, 0.1 eq) and K₂CO₃ (648 mg, 4.69 mmol, 3.0 eq) in dioxane (30 mL) and H₂O (7.5 mL) was degassed and purged with N₂ three times, and then the mixture was stirred at 80° C. for 12 h under N₂. The reaction mixture was diluted with H₂O (10 mL) and extracted with EtOAc (10 mL×3). The combined organic layers were washed with brine (10 mL), dried over Na₂SO₄, filtered, and concentrated under reduced pressure to give a residue, which was purified by column chromatography (SiO₂, Dichloromethane:Methanol:NH₃·H₂O (10:1:0.001)=1/0/0 to 10/1/0.001) to give the title compound (100 mg, 311 μmol, 19.90% yield, 80% purity) as dark brown oil. ¹H NMR: (400 MHz, CDCl₃) δ ppm: 7.90 (s, 1H), 7.45 (s, 1H), 7.36 (s, 1H), 6.87 (s, 1H), 6.61 (t, J=4.4 Hz, 1H), 3.82 (s, 3H), 3.55 (t, J=7.2 Hz, 2H), 3.06 (s, 3H), 2.57-2.49 (m, 2H).

3-(7-methoxyimidazo[1,2-a]pyridin-6-yl)-1-methylpiperidin-2-one: To a solution of 3-(7-methoxyimidazo[1,2-a]pyridin-6-yl)-1-methyl-5,6-dihydropyridin-2 (1H)-one (100 mg, 311 μmol, 1 eq) in EtOAc (20 mL) was added Pd/C (39.7 mg, 37.3 μmol, 10% purity, 0.12 eq) at 40° C. The mixture was stirred at 40° C. for 4 h under H₂ (40 Psi) atmosphere. The reaction mixture was filtered and concentrated under reduced pressure to give the title compound (90 mg, crude) as dark brown oil, which was used without further purification. [M+H]⁺=260.0.

### Intermediate 123

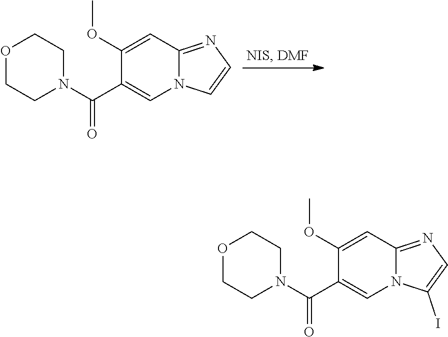 [SMILES: COc1cc2nccn2cc1C(=O)N1CCOCC1 ; COc1cc2ncc(I)n2cc1C(=O)N1CCOCC1]

(3-iodo-7-methoxyimidazo[1,2-a]pyridin-6-yl)(morpholino)methanone: To a solution of (7-methoxyimidazo[1,2-a]pyridin-6-yl)(morpholino)methanone (180 mg, 689 μmol, 1 eq) in DMF (10 mL) was added NIS (310 mg, 1.38 mmol, 2 eq) at 0° C. The mixture was stirred at 25° C. for 12 h, after which it was evaporated under reduced pressure. The residue was purified by flash silica gel chromatography (from DCM/MeOH=100/0 to 90/10) to yield the title compound (180 mg, 325 μmol, 47% yield, 70% purity) as yellow oil. ¹H NMR: (400 MHz, CDCl₃) δ ppm: 8.09 (s, 1H), 7.60 (s, 1H), 7.04 (s, 1H), 3.94 (s, 3H), 3.72-3.57 (m, 8H).

The following compounds were prepared according to procedures analogous to that described for Intermediate 123.

| Int. | Structure | Name | LCMS, ¹H NMR |
|---|---|---|---|
| 124 | 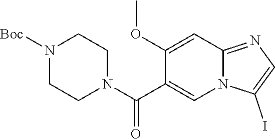 [SMILES: COc1cc2ncc(I)n2cc1C(=O)N1CCN(C(=O)OC(C)(C)C)CC1] | tert-butyl 4-(3-iodo-7- methoxyimidazo[1,2- a]pyridine-6- carbonyl)piperazine-1- carboxylate | ¹H NMR: (400 MHZ, CDCl₃) δ ppm: 8.08 (s, 1H), 7.59 (s, 1H), 6.99 (s, 1H), 3.92 (s, 3H), 3.66- 3.17 (m, 8H), 1.48 (s, 9H). |
| 125 | 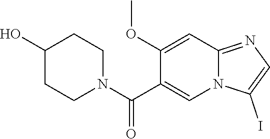 [SMILES: COc1cc2ncc(I)n2cc1C(=O)N1CCC(O)CC1] | (4-hydroxypiperidin-1- yl)(3-iodo-7- methoxyimidazo[1,2- a]pyridin-6- yl)methanone | [M + H]⁺ = 401.8 |
| 126 | 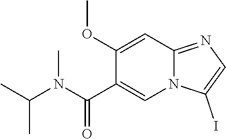 [SMILES: COc1cc2ncc(I)n2cc1C(=O)N(C)C(C)C] | 3-iodo-N-isopropyl-7- methoxy-N- methylimidazo[1,2- a]pyridine-6- carboxamide | [M + H]⁺ = 374.0 |
| 127 | 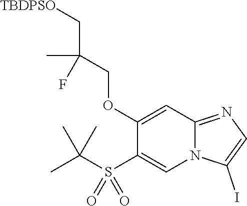 | 7-(3-((tert- butyldiphenylsilyl)oxy)- 2-fluoro-2- methylpropoxy)-6- (tert-butylsulfonyl)-3- iodoimidazo[1,2-a] pyridine | [M + H]⁺ = 709.1 ¹H NMR: (400 MHZ, DMSO-d₆) δ ppm: 8.47 (s, 1H), 7.73 (s, 1H), 7.64- 7.62 (m, 4H), 7.46-7.38 (m, 6H), 7.31 (s, 1H), 4.40- 4.26 (m, 2H), 4.08-4.04 (m, 1H), 3.94-3.86 (m, 1H), 1.54-1.24 (m, 3H), 1.26 (s, 9H), 1.01 (s, 9H). |
| 128 | 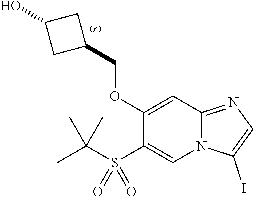 [SMILES: CC(C)(C)S(=O)(=O)c1cn2c(I)cnc2cc1OC[C@H]1C[C@H](O)C1] | (1r,3r)-3-(((6-(tert- butylsulfonyl)-3- iodoimidazo[1,2- a]pyridin-7-yl)oxy) methyl)cyclobutan-1-ol | ¹H NMR: (400 MHz, DMSO-d₆) δ ppm: 8.50 (s, 1H), 7.71 (s, 1H), 7.29- 7.20 (m, 1H), 5.01 (d, J = 6.0 Hz, 1H), 4.36-4.25 (m, 1H), 4.14 (d, J = 7.2 Hz, 2H), 2.19-2.07 (m, 2H), 2.05-1.94 (m, 2H), 1.75- 1.50 (m, 1H), 1.36 (s, 9H). |
| 129 | 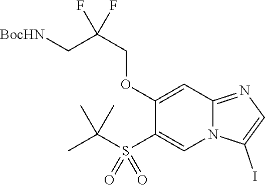 [SMILES: CC(C)(C)OC(=O)NCC(F)(F)COc1cc2ncc(I)n2cc1S(=O)(=O)C(C)(C)C] | tert-butyl (3-((6-(tert- butylsulfonyl)-3- iodoimidazo[1,2- a]pyridin-7-yl)oxy)- 2,2-difluoropropyl)carbamate | ¹H NMR: (400 MHZ, DMSO-d₆) δ ppm: 8.51 (s, 1H), 7.76 (s, 1H), 7.37 (s, 1H), 7.28 (t, J = 4.8 Hz, 1H), 4.48 (t, J = 12.4 Hz, 2H), 3.71-3.61 (m, 2H), 1.38 (s, 9H), 1.33 (s, 9H). |
| 130 | 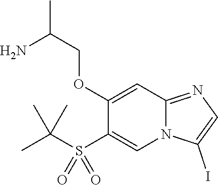 [SMILES: CC(N)COc1cc2ncc(I)n2cc1S(=O)(=O)C(C)(C)C] | 1-((6-(tert- butylsulfonyl)-3- iodoimidazo[1,2- a]pyridin-7- yl)oxy)propan-2-amine | ¹H NMR: (400 MHZ, DMSO-d₆) δ ppm: 8.50 (s, 1H), 7.77 (s, 1H), 7.41 (s, 1H), 4.26-4.23 (m, 1H), 4.18-4.15 (m, 1H), 3.62- 3.56 (m, 1H), 1.35 (s, 9H), 1.29 (d, J = 6.4 Hz, 3H). |
| 131 | 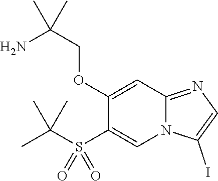 [SMILES: CC(C)(N)COc1cc2ncc(I)n2cc1S(=O)(=O)C(C)(C)C] | 1-((6-(tert- butylsulfonyl)-3- iodoimidazo[1,2- a]pyridin-7-yl)oxy)-2- methylpropan-2-amine | ¹H NMR: (400 MHZ, DMSO-d₆) δ ppm: 8.49 (s, 1H), 7.74 (s, 1H), 7.32- 7.25 (m, 1H), 3.94 (s, 2H), 3.18-3.15 (m, 2H), 1.35 (s, 9H), 1.20 (s, 6H). |
| 132 | 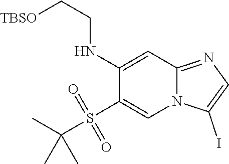 [SMILES: CC(C)(C)[Si](C)(C)OCCNc1cc2ncc(I)n2cc1S(=O)(=O)C(C)(C)C] | N-(2-((tert- butyldimethylsilyl)oxy) ethyl)-6-(tert- butylsulfonyl)-3- iodoimidazo[1,2-a] pyridin-7-amine | [M + H]⁺ = 538.0. ¹H NMR: (400 MHZ, DMSO-d₆) δ ppm: 8.44 (s, 1H), 7.51 (s, 1H), 6.81- 6.73 (m, 1H), 6.55 (s, 1H), 3.88 (t, J = 5.2 Hz, 2H), 3.28-3.21 (m, 2H), 1.41 (s, 9H), 0.92 (s, 9H), 0.10 (s, 6H). |
| 133 | 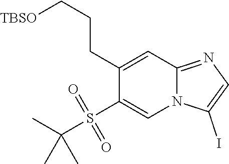 [SMILES: CC(C)(C)[Si](C)(C)OCCCc1cc2ncc(I)n2cc1S(=O)(=O)C(C)(C)C] | 7-(3-((tert- butyldimethylsilyl)oxy) propyl)-6-(tert- butylsulfonyl)-3- iodoimidazo[1,2-a] pyridine | [M + H]⁺ = 537.1 |
| 134 | 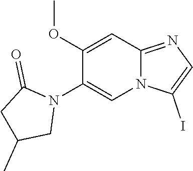 [SMILES: COc1cc2ncc(I)n2cc1N1CC(C)CC1=O] | 1-(3-iodo-7- methoxyimidazo[1,2- a]pyridin-6- yl)pyrrolidin-2-one | ¹H NMR: (400 MHZ, CDCl₃) δ ppm: 8.03 (s, 1H), 7.55 (s, 1H), 6.97 (s, 1H), 3.91 (s, 3H), 3.77 (t, J = 7.2 Hz, 2H), 2.60 (t, J = 8.0 Hz, 2H), 2.25 (t, J = 7.2 Hz, 2H). |
| 135 | 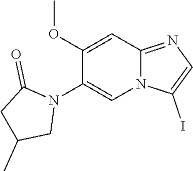 [SMILES: COc1cc2ncc(I)n2cc1N1CC(C)CC1=O] | 1-(3-iodo-7- methoxyimidazo[1,2- a]pyridin-6-yl)-4- methylpyrrolidin-2-one | [M + H]⁺ = 372.0 |
| 136 | 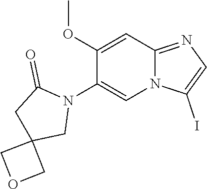 [SMILES: COc1cc2ncc(I)n2cc1N1CC2(COC2)CC1=O] | 6-(3-iodo-7- methoxyimidazo[1,2- a]pyridin-6-yl)-2-oxa- 6-azaspiro[3.4]octan-7- one | ¹H NMR: (400 MHZ, CDCl₃) δ ppm: 8.03 (s, 1H), 7.57 (s, 1H), 7.02 (s, 1H), 4.83-4.79 (m, 2H), 4.77-4.72 (m, 2H), 4.05 (s, 2H), 3.93-3.89 (m, 3H), 2.92 (s, 2H). |
| 137 | 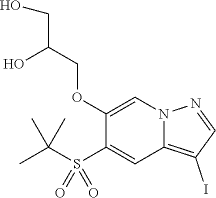 [SMILES: CC(C)(C)S(=O)(=O)c1cc2c(I)cnn2cc1OCC(O)CO] | 3-((5-(tert- butylsulfonyl)-3- iodopyrazolo[1,5-a] pyridin-6- yl)oxy)propane-1,2-diol | ¹H NMR: (400 MHZ, DMSO-d₆) δ ppm: 8.79 (s, 1H), 8.21 (s, 1H), 7.87 (s, 1H), 4.89 (d, J = 4.8 Hz, 1H), 4.64 (t, J = 5.6 Hz, 1H), 4.13-4.09 (m, 1H), 4.07-4.02 (m, 1H), 3.86- 3.81 (m, 1H), 3.53-3.46 (m, 2H), 1.33 (s, 9H). |
| 138 | 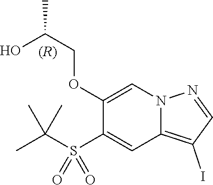 | (R)-1-((5-(tert- butylsulfonyl)-3- iodopyrazolo[1,5-a] pyridin-6- yl)oxy)propan-2-ol | ¹H NMR: (400 MHZ, DMSO-d₆) δ ppm: 8.79 (s, 1H), 8.22 (s, 1H), 7.87 (s, 1H), 4.81 (d, J = 4.0 Hz, 1H), 4.03-3.90 (m, 3H), 1.33 (s, 9H), 1.20 (d, J = 6.0 Hz, 3H). |
| 139 | 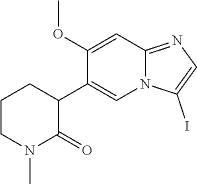 [SMILES: COc1cc2ncc(I)n2cc1C1CCCN(C)C1=O] | 3-(3-iodo-7- methoxyimidazo[1,2- a]pyridin-6-yl)-1- methylpiperidin-2-one | ¹H NMR: (400 MHZ, CDCl₃) δ ppm: 7.80 (s, 1H), 7.50 (s, 1H), 6.90 (s, 1H), 3.87 (s, 3H), 3.71- 3.61 (m, 1H), 3.55-3.45 (m, 1H), 3.41-3.32 (m, 1H), 3.04 (s, 3H), 2.15-2.06 (m, 2H), 2.01-1.87 (m, 2H). |
| 140 | 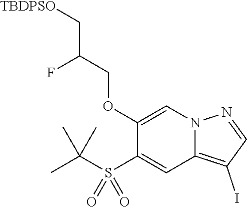 | 7-(3-((tert- butyldiphenylsilyl)oxy)- 2-fluoropropoxy)-6- (tert-butylsulfonyl)-3- iodoimidazo[1,2-a] pyridine | [M + H]⁺ = 695.1. ¹H NMR: (400 MHZ, DMSO-d₆) δ ppm: 8.48 (s, 1H), 7.73 (s, 1H), 7.67- 7.63 (m, 4H), 7.47-7.40 (m, 6H), 7.29 (s, 1H), 5.11- 4.93 (m, 1H), 4.51-4.34 (m, 2H), 4.19-4.03 (m, 2H), 1.26 (s, 9H), 1.01 (s, 9H). |

### Intermediate 141

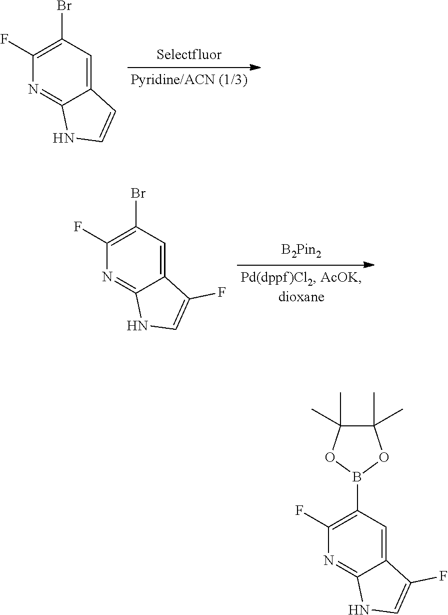 [SMILES: Fc1nc2[nH]ccc2cc1Br ; Fc1nc2[nH]cc(F)c2cc1Br ; CC1(C)OB(c2cc3c(F)c[nH]c3nc2F)OC1(C)C]

5-bromo-3,6-difluoro-1H-pyrrolo[2,3-b]pyridine: To a solution of 5-bromo-6-fluoro-1H-pyrrolo[2,3-b]pyridine (200 mg, 930 μmol, 1 eq) in ACN (1 mL) and pyridine (0.3 mL) was added selectfluor (395 mg, 1.12 mmol, 1.2 eq) at 0° C. The mixture was stirred at 40° C. for 12 h, after which it was partitioned between EtOAc (30 mL) and H₂O (10 mL). The organic phase was separated, washed with brine (10 mL×3), dried over Na₂SO₄, filtered, and concentrated under reduced pressure to give a residue, which was purified by prep-HPLC (water (NH₄HCO₃)—ACN) to give the title compound (30 mg, 116 μmol, 12.5% yield, 90% purity) as a red solid. ¹H NMR (400 MHz, CDCl₃) δ ppm: 8.32 (s, 1H), 8.21 (d, J=8.4 Hz, 1H), 7.03 (t, J=2.4 Hz, 1H).

3,6-difluoro-5-(4,4,5,5-tetramethyl-1,3,2-dioxaborolan-2-yl)-1H-pyrrolo[2,3-b]pyridine: A mixture of 5-bromo-3,6-difluoro-1H-pyrrolo[2,3-b]pyridine (30 mg, 116 μmol, 1 eq), B₂Pin₂ (58.9 mg, 232 μmol, 2 eq), KOAc (34.1 mg, 348 μmol, 3 eq), and Pd(dppf)Cl₂ (8.48 mg, 11.6 μmol, 0.1 eq) in dioxane (0.5 mL) was degassed and purged with N₂ three times, and then the mixture was stirred at 100° C. for 2 h under N₂. The reaction mixture was concentrated under reduced pressure to give a residue, which was purified by flash silica gel chromatography (0˜10% Ethyl acetate/Petroleum ether) to give the title compound (20 mg, 64.3 μmol, 55.5% yield, 90% purity) as a white solid. ¹H NMR: ES20061-1118-P1A (400 MHz, CDCl₃) δ ppm: 8.57 (s, 1H), 8.48 (d, J=8.0 Hz, 1H), 6.97 (s, 1H), 1.39 (s, 12H).

The following compounds were prepared following procedures analogous to those described for Intermediate 141.

| Int. | Structure | Name | LCMS, ¹H NMR |
|---|---|---|---|
| 142 | 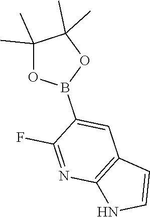 [SMILES: CC1(C)OB(c2cc3cc[nH]c3nc2F)OC1(C)C] | 6-fluoro-5-(4,4,5,5- tetramethyl-1,3,2- dioxaborolan-2-yl)-1H- pyrrolo[2,3-b]pyridine | [M + H]⁺ = 263.1 |
| 143 | 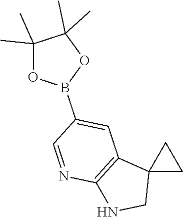 [SMILES: CC1(C)OB(c2cnc3c(c2)C2(CC2)CN3)OC1(C)C] | 5′-(4,4,5,5-tetramethyl- 1,3,2-dioxaborolan-2-yl)- 1′,2′-dihydrospiro [cyclopropane-1,3′- pyrrolo[2,3-b] pyridine] | [M + H]⁺ = 273.1 |

The following compounds were prepared following procedures analogous to those described for Intermediate 99.

| Int. No. | Structure | Name | LCMS, ¹H NMR |
|---|---|---|---|
| 144 | 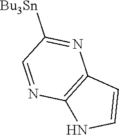 | 2- (tributylstannyl)- 5H-pyrrolo[2,3- b]pyrazine | ¹H NMR (400 MHZ, DMSO-d₆) δ ppm 11.85 (s, 1H), 8.14 (s, 1H), 7.77 (t, J = 3.2 Hz, 1H), 6.62 (dd, J = 1.6, 3.2 Hz, 1H), 1.64-1.49 (m, 6H), 1.33- 1.25 (m, 6H), 1.16-1.03 (m, 6H), 0.89-0.80 (m, 9H). |

### Intermediate 145

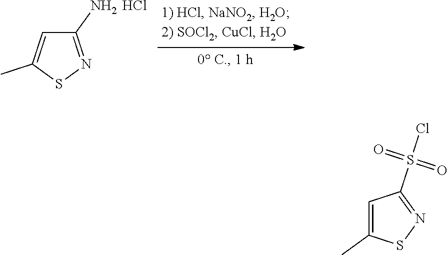

5-methylisothiazole-3-sulfonyl chloride: To H₂O (15 mL) cooled to 0° C. was added SOCl₂ (3.40 g, 28.6 mmol, 2.07 mL, 4.3 eq) keeping the temperature at 0° C., the mixture was allowed to warm to 25° C. for 1 h, after which CuCl (65.7 mg, 664 μmol, 15.9 μL, 0.1 eq) was added and the solution cooled to 0° C. Separately, NaNO₂ (527 mg, 7.63 mmol, 1.15 eq) was added to H₂O (2.25 mL) keeping the temperature below 25° C., and the mixture was cooled to 0° C. 5-methylisothiazol-3-amine HCl salt (1 g, 6.64 mmol, 1 eq) dissolved in HCl (12 M, 553 μL, 1 eq) was added dropwise keeping the temperature at 0-5° C., and the mixture was stirred 15 min. The resulting slurry was added dropwise to the thionyl chloride solution while keeping the temperature at 0° C., and this mixture stirred for 30 min. The reaction mixture was diluted with H₂O (20 mL) and extracted with DCM (20 mL×3), dried over Na₂SO₄, filtered, and concentrated under reduced pressure to give a residue, which was purified by column chromatography (SiO₂, PE/EtOAc=100/0 to 9/1) to yield the title compound (160 mg, 729 μmol, 11% yield, 90% purity) as yellow liquid. ¹H NMR: (400 MHz, DMSO-d₆) δ ppm: 7.35 (s, 1H), 2.40 (s, 3H).

### Intermediate 146

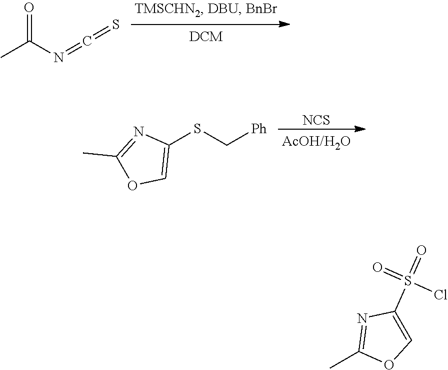

4-(benzylthio)-2-methyloxazole: To a stirred solution of acetyl isothiocyanate (300 mg, 2.97 mmol, 261 μL, 1 eq) in DCM (9 mL) was added dropwise TMSCHN₂ (2 M, 2.22 mL, 1.5 eq) at 0° C. and stirred at 0° C. for 1 h. To the reaction mixture was added dropwise DBU (903 mg, 5.93 mmol, 894 μL, 2 eq) and BnBr (507 mg, 2.97 mmol, 352 μL, 1 eq), and the mixture was stirred at 25° C. for 11 h. The reaction mixture was diluted with H₂O (10 mL) and extracted with DCM (20 mL×3). The combined organic layers were washed with brine (10 mL), dried over Na₂SO₄, filtered, and concentrated under reduced pressure to give a residue, which was purified by prep-TLC (PE/EtOAc=5/1) to yield the title compound (170 mg, 778 μmol, 26.2% yield, 94% purity) as yellow oil. [M+H]⁺=206.0. ¹H NMR: (400 MHz, DMSO-d₆) δ ppm: 7.87 (s, 1H), 7.31-7.25 (m, 5H), 4.06 (s, 2H), 2.40 (s, 3H).

2-methyloxazole-4-sulfonyl chloride: To a stirred solution of 4-(benzylthio)-2-methyloxazole (170 mg, 778 μmol, 1 eq) in AcOH (2 mL) and H₂O (0.5 mL) was added NCS (260 mg, 1.95 mmol, 2.5 eq) at 0° C. The reaction mixture was stirred at 25° C. for 2 h, after which it was diluted with H₂O (5 mL) and extracted with EtOAc (10 mL×3). The combined organic layers were washed with brine (5 mL), dried over Na₂SO₄, filtered, and concentrated to give a residue, which was purified by prep-TLC (PE/EtOAc=5/1) to yield the title compound (127 mg, 349 μmol, 44.9% yield, 50% purity) as white oil. ¹H NMR: (400 MHz, DMSO-d₆) δ ppm: 7.90 (s, 1H), 2.38 (s, 3H).

### EXAMPLES

### Example 1

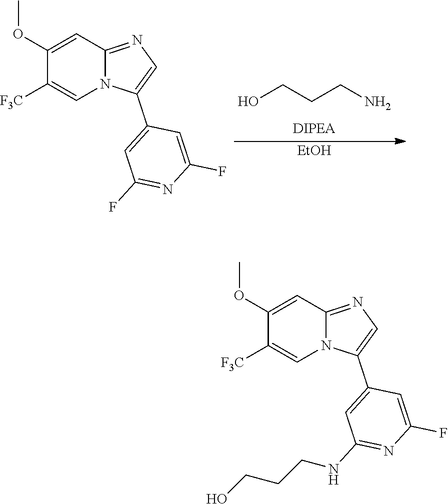 [SMILES: "EtOH" ; COc1cc2ncc(-c3cc(F)nc(F)c3)n2cc1C(F)(F)F ; NCCCO ; COc1cc2ncc(-c3cc(F)nc(NCCCO)c3)n2cc1C(F)(F)F]

3-((6-Fluoro-4-(7-methoxy-6-(trifluoromethyl)imidazo[1,2-a]pyridin-3-yl)pyridin-2-yl)amino)propan-1-ol: To a stirred solution of 3-(2,6-difluoropyridin-4-yl)-7-methoxy-6-(trifluoromethyl)imidazo[1,2-a]pyridine (10 mg, 0.030 mmol) in EtOH was added DIPEA (0.016 mL, 0.091 mmol) followed by 3-aminopropan-1-ol (2.96 mg, 0.039 mmol) at 0° C. under a N₂ atmosphere. The reaction mixture was heated at 80° C. for 16 h before it was concentrated under vacuum. The crude compound was purified by RP-HPLC to afford the title compound (3.2 mg, 13.6%) as a white solid. [M+H]⁺=385.33. ¹H NMR (400 MHz, DMSO-d₆) δ 8.75 (s, 1H), 7.90 (s, 1H), 7.33 (s, 1H), 7.13 (s, 1H), 6.61 (s, 1H), 6.46 (s, 1H), 4.48 (s, 1H), 3.98 (s, 3H), 3.32-3.26 (m, 2H), 1.73-1.66 (m, 2H).

The following compounds were prepared following procedures analogous to that described for Example 1.

| Ex. | | | |
|---|---|---|---|
| No. | Structure | Name | LCMS, ¹H NMR |
| 2 | 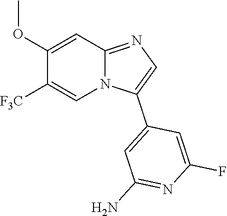 [SMILES: COc1cc2ncc(-c3cc(N)nc(F)c3)n2cc1C(F)(F)F] | 6-fluoro-4-(7-methoxy-6- (trifluoromethyl) imidazo[1,2-a]pyridin-3- yl)pyridin-2-amine | [M + H]⁺ = 327.2 ¹H NMR (400 MHZ, DMSO-d₆) δ ppm 8.74 (s, 1H), 7.89 (s, 1H), 7.33 (s, 1H), 6.56 (d, J = 6.8 Hz, 2H), 6.50 (s, 1H), 3.98 (s, 1H) |
| 3 | 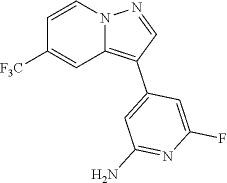 [SMILES: Nc1cc(-c2cnn3ccc(C(F)(F)F)cc23)cc(F)n1] | 6-fluoro-4-(5-(trifluoromethyl) pyrazolo[1,5-a]pyridin-3- yl)pyridin-2-amine | [M + H]⁺ = 297.2 ¹H NMR (400 MHZ, DMSO-d₆) δ ppm 9.02 (d, J = 7.2 Hz, 1H), 8.67 (s, 1H), 8.30 (s, 1H), 7.28 (dd, J = 7.2, 1.6 Hz, 1H), 6.71 (s, 1H), 6.56 (s, 1H), 6.40 (s, 2H) |
| 4 | 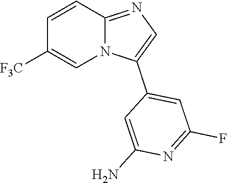 [SMILES: Nc1cc(-c2cnc3ccc(C(F)(F)F)cn23)cc(F)n1] | 6-fluoro-4-(6-(trifluoromethyl) imidazo[1,2-a]pyridin-3- yl)pyridin-2-amine | [M + H]⁺ = 297.0 ¹H NMR (400 MHZ, DMSO-d₆) δ ppm 8.92 (s, 1H), 8.07 (s, 1H), 7.90 (d, J = 9.2 Hz, 1H), 7.60 (dd, J = 9.2, 1.6 Hz, 1H), 6.64-6.55 (m, 3H), 6.54 (s, 1H). |
| 5 | 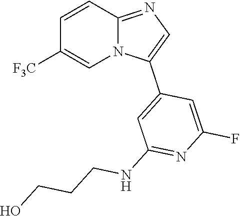 [SMILES: OCCCNc1cc(-c2cnc3ccc(C(F)(F)F)cn23)cc(F)n1] | 3-((6-fluoro-4-(6- (trifluoromethyl) imidazo[1,2-a]pyridin-3- yl)pyridin-2- yl)amino)propan-1-ol | [M + H]⁺ = 355.1 ¹H NMR (400 MHZ, DMSO-d₆) δ ppm 8.93 (s, 1H), 8.08 (s, 1H), 7.90 (d, J = 9.6 Hz, 1H), 7.60 (dd, J = 1.6, 9.2 Hz, 1H), 7.18 (t, J = 4.8 Hz, 1H), 6.66 (s, 1H), 6.51 (s, 1H), 4.50 (t, J = 5.2 Hz, 1H), 3.50 (q, J = 5.2 Hz, 2H), 3.34-3.25 (m, 2H), 1.76- 1.64 (m, 2H). |
| 6 | 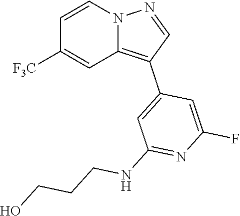 [SMILES: OCCCNc1cc(-c2cnn3ccc(C(F)(F)F)cc23)cc(F)n1] | 3-((6-fluoro-4-(5- (trifluoromethyl) pyrazolo[1,5-a]pyridin-3- yl)pyridin-2- yl)amino)propan-1-ol | [M + H]⁺ = 355.3 ¹H NMR (400 MHZ, DMSO-d₆) δ ppm 9.02 (d, J = 7.2 Hz, 1H), 8.68 (s, 1H), 8.31 (s, 1H), 7.28 (dd, J = 7.2, 1.6 Hz, 1H), 6.96 (t, J = 5.2 Hz, 1H), 6.73 (s, 1H), 6.53 (s, 1H), 4.48 (s, 1H), 3.51 (q, J = 5.9 Hz, 2H), 3.28 (d, J = 6.8 Hz, 2H), 1.74-1.67 (m, 2H). |

### Example 7

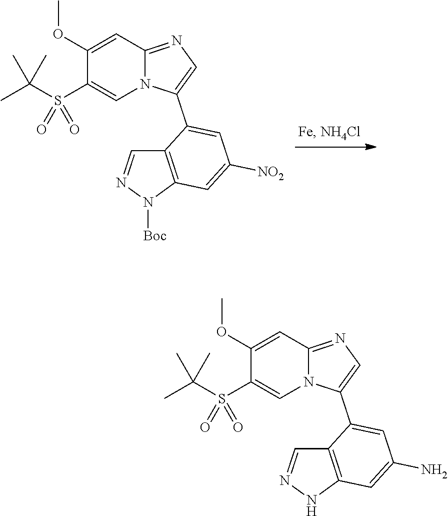 [SMILES: COc1cc2ncc(-c3cc([N+](=O)[O-])cc4c3cnn4C(=O)OC(C)(C)C)n2cc1S(=O)(=O)C(C)(C)C ; COc1cc2ncc(-c3cc(N)cc4[nH]ncc34)n2cc1S(=O)(=O)C(C)(C)C]

4-[7-Methoxy-6-(2-methylpropane-2-sulfonyl)imidazo[1,2-a]pyridin-3-yl]-1H-indazol-6-amine: To a mixture of tert-butyl 4-(6-(tert-butylsulfonyl)-7-methoxyimidazo[1,2-a]pyridin-3-yl)-6-nitro-1H-indazole-1-carboxylate (170 mg, 321 μmol) and Fe powder (89.6 mg, 1.60 mmol) in water (4.5 mL) and EtOH (15 mL) was added NH₄Cl (85.8 mg, 1.60 mmol). The mixture was stirred at 80° C. for 4 h before the solids were removed by filtration through Celite®. The filter cake was rinsed with EtOAc and the filtrate was concentrated. The crude material was stirred in acetone, and then the precipitate was collected by filtration before it was purified by column chromatography (SiO₂, DCM/EtOH) followed by RP-HPLC to give the title compound (16.2 mg, 11% yield). [M+H]⁺=400.1. H NMR (400 MHz, DMSO-d₆) δ ppm 12.58 (s, 1H), 8.58 (s, 1H), 7.80 (s, 1H), 7.61 (s, 1H), 7.31 (s, 1H), 6.67 (d, J=1.8 Hz, 1H), 6.64-6.62 (m, 1H), 5.51 (br s, 2H), 3.95 (s, 3H), 1.30 (s, 9H).

### Example 8

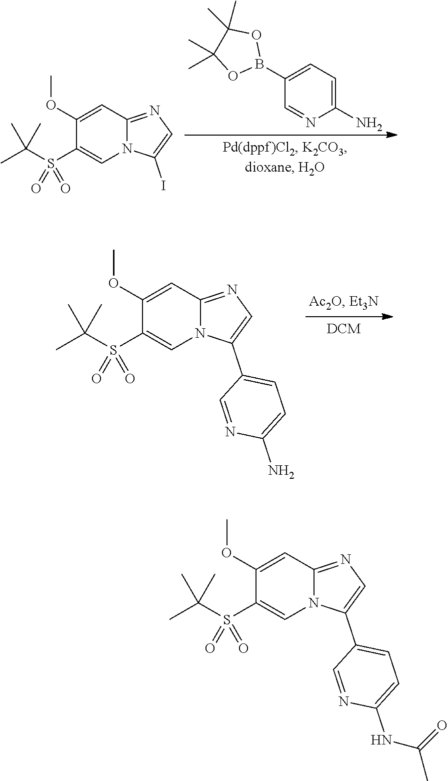 [SMILES: COc1cc2ncc(I)n2cc1S(=O)(=O)C(C)(C)C ; CC1(C)OB(c2ccc(N)nc2)OC1(C)C ; COc1cc2ncc(-c3ccc(N)nc3)n2cc1S(=O)(=O)C(C)(C)C ; COc1cc2ncc(-c3ccc(NC(C)=O)nc3)n2cc1S(=O)(=O)C(C)(C)C]

5-(6-(tert-butylsulfonyl)-7-methoxyimidazo[1,2-a]pyridin-3-yl)pyridin-2-amine: To a solution of 5-(4,4,5,5-tetramethyl-1,3,2-dioxaborolan-2-yl)pyridin-2-amine (50 mg, 227.20 μmol, 1 eq) and 6-(tert-butylsulfonyl)-3-iodo-7-methoxyimidazo[1,2-a]pyridine (119.42 mg, 272.63 μmol, 90% purity, 1.2 eq) in dioxane (4 mL) and H₂O (1 mL) was added K₂CO₃ (94.20 mg, 681.59 μmol, 3 eq) and Pd(dppf)Cl₂ (16.62 mg, 22.72 μmol, 0.1 eq) under N₂. The mixture was stirred at 100° C. for 2.5 hours under a N₂ atmosphere before it was diluted with water (20 mL) and extracted with EtOAc (3×25 mL). The combined organic phase was dried over Na₂SO₄, filtered and concentrated to give a residue. The residue was purified by RP-HPLC to give the title compound (3.3 mg, 4% yield) as a yellow solid. [M+H]⁺=360.9. ¹H NMR (400 MHz, DMSO-d₆) δ ppm 8.50 (s, 1H), 8.10 (d, J=2.0 Hz, 1H), 7.64-7.56 (m, 2H), 7.22 (s, 1H), 6.61 (d, J=8.4 Hz, 1H), 6.32 (s, 2H), 3.91 (s, 3H), 1.29 (s, 9H).

N-(5-(6-(tert-butylsulfonyl)-7-methoxyimidazo[1,2-a]pyridin-3-yl)pyridin-2-yl)acetamide: To a solution of 5-(6-(tert-butylsulfonyl)-7-methoxyimidazo[1,2-a]pyridin-3-yl)pyridin-2-amine (20 mg, 49.94 μmol, 90% purity, 1 eq) in DCM (1.5 mL) was added Ac₂O (5.10 mg, 49.94 μmol, 4.68 μL, 1 eq) and Et₃N (5.05 mg, 49.94 μmol, 6.95 μL, 1 eq). The mixture was stirred at 40° C. for 12 hours before it was filtered and concentrated to give a residue, which was purified by RP-HPLC to give the title compound (1.8 mg, 60% yield) as a white solid. [M+H]⁺=403.1. ¹H NMR (400 MHz, DMSO-d₆) δ ppm 10.70 (s, 1H), 8.61 (s, 1H), 8.55 (d, J=2.4 Hz, 1H), 8.25 (d, J=8.8 Hz, 1H), 8.04 (dd, J=2.4, 8.8 Hz, 1H), 7.80 (s, 1H), 7.27 (s, 1H), 3.94 (s, 3H), 2.14 (s, 3H), 1.31 (s, 9H).

### Example 9

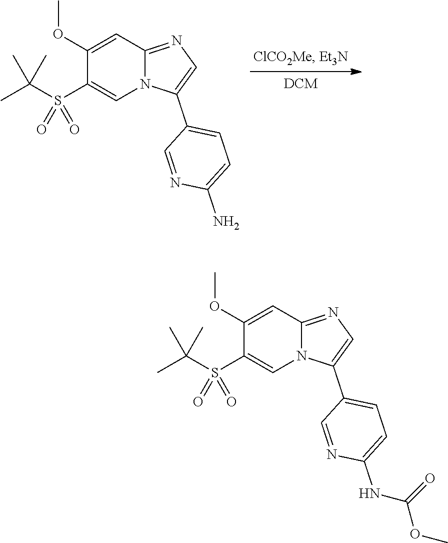 [SMILES: COc1cc2ncc(-c3ccc(N)nc3)n2cc1S(=O)(=O)C(C)(C)C ; COC(=O)Nc1ccc(-c2cnc3cc(OC)c(S(=O)(=O)C(C)(C)C)cn23)cn1]

methyl (5-(6-(tert-butylsulfonyl)-7-methoxyimidazo[1,2-a]pyridin-3-yl)pyridin-2-yl)carbamate: To a stirred solution of 5-(6-(tert-butylsulfonyl)-7-methoxyimidazo[1,2-a]pyridin-3-yl)pyridin-2-amine (40 mg, 99.88 μmol, 90% purity, 1 eq) and Et₃N (30.32 mg, 299.64 μmol, 41.71 μL, 3 eq) in DCM (4 mL) was added methyl chloroformate (18.88 mg, 199.76 μmol, 15.47 μL, 2 eq) at 0° C. The reaction mixture was stirred at 25° C. for 0.3 h under N₂ before it was diluted with H₂O (15 mL) and extracted with EtOAc (3×20 mL). The combined organic layers were washed with brine (20 mL), dried over Na₂SO₄, filtered and concentrated under reduced pressure to give a residue. The residue was purified by RP-HPLC to give compound (3.67 mg, 9% yield) as a white solid. [M+H]⁺=419.1. ¹H NMR (400 MHz, DMSO-d₆) δ ppm 10.45 (s, 1H), 8.60 (s, 1H), 8.51 (d, J=1.2 Hz, 1H), 8.08-7.97 (m, 2H), 7.78 (s, 1H), 7.27 (s, 1H), 3.94 (s, 3H), 3.72 (s, 3H), 1.31 (s, 9H).

### Example 10

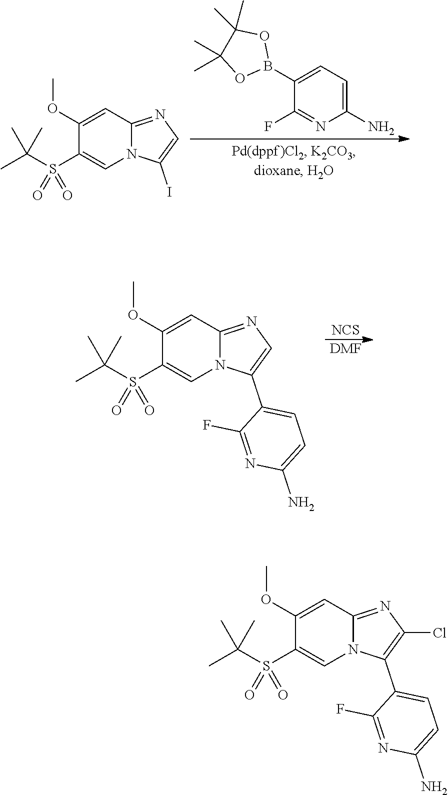

5-(6-(tert-butylsulfonyl)-7-methoxyimidazo[1,2-a]pyridin-3-yl)-6-fluoropyridin-2-amine: A mixture of 6-fluoro-5-(4,4,5,5-tetramethyl-1,3,2-dioxaborolan-2-yl)pyridin-2-amine (50 mg, 210.03 μmol, 1 eq), 6-(tert-butylsulfonyl)-3-iodo-7-methoxyimidazo[1,2-a]pyridine (92.00 mg, 210.03 μmol, 90% purity, 1.0 eq), K₂CO₃ (72.57 mg, 525.07 μmol, 2.5 eq) in 1,4-dioxane (1.0 mL) and H₂O (0.2 mL) was degassed and purged with N₂ 3 times, and then Pd(dppf)Cl₂ (15.37 mg, 21.00 μmol, 0.1 eq) was added. The mixture was stirred at 90° C. for 12 hours before it was filtered and concentrated under reduced pressure. The crude product was purified by RP-HPLC to give the title compound (12.9 mg, 16% yield) as a white solid. [M+H]⁺=379.0. ¹H NMR (400 MHz, DMSO-d₆) δ ppm 8.30 (d, J=2.4 Hz, 1H), 7.67 (dd, J=10.0, 8.4 Hz, 1H), 7.63 (s, 1H), 7.25 (s, 1H), 6.79 (s, 2H), 6.49 (dd, J=8.0, 2.0 Hz, 1H), 3.93 (s, 3H), 1.29 (s, 9H).

5-(6-(tert-butylsulfonyl)-2-chloro-7-methoxyimidazo[1,2-a]pyridin-3-yl)-6-fluoropyridin-2-amine: To a solution of 5-(6-(tert-butylsulfonyl)-7-methoxyimidazo[1,2-a]pyridin-3-yl)-6-fluoropyridin-2-amine (6 mg, 15.86 μmol, 1 eq) in DMF (0.5 mL) was added NCS (2.54 mg, 19.03 μmol, 1.2 eq). The mixture was stirred at 50° C. for 3.5 hours, and then more NCS (0.6 mg) was added. The resulting mixture was stirred at 50° C. for another 3 hours before it was diluted with EtOAc (15 mL), washed with brine (2×6 mL), dried over Na₂SO₄, and filtered. The filtrate was concentrated under reduced pressure to give a residue, which was purified by RP-HPLC to give the title compound (1.03 mg, 13% yield) as a white solid. [M+H]⁺=413.1. ¹H NMR (400 MHz, DMSO-d₆) δ ppm 8.17 (d, J=1.6 Hz, 1H), 7.71-7.65 (m, 1H), 7.25 (s, 1H), 6.92 (s, 2H), 6.52 (dd, J=8.0, 1.6 Hz, 1H), 3.93 (s, 3H), 1.28 (s, 9H).

### Example 11

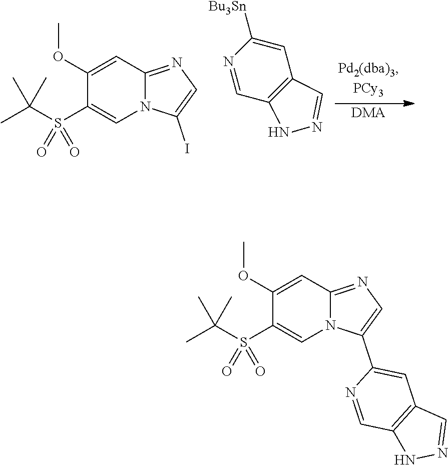

5-(6-(tert-butylsulfonyl)-7-methoxyimidazo[1,2-a]pyridin-3-yl)-1H-pyrazolo[3,4-c]pyridine: To a solution of 5-(tributylstannyl)-1H-pyrazolo[3,4-c]pyridine (100 mg, 220.50 μmol, 1 eq), 6-(tert-butylsulfonyl)-3-iodo-7-methoxyimidazo[1,2-a]pyridine (96.58 mg, 220.50 μmol, 1 eq) in DMA (5 mL) was added PCy₃ (7.42 mg, 26.46 μmol, 8.58 μL, 0.12 eq) and Pd₂(dba)₃ (20.19 mg, 22.05 μmol, 0.1 eq), and then the mixture was degassed and purged with N₂ 3 times. The mixture was stirred at 80° C. for 12 h under a N₂ atmosphere before it was diluted with H₂O (20 mL) and extracted with EtOAc (3×20 mL). The combined organic layers were dried over Na₂SO₄, filtered and concentrated under reduced pressure to give a residue, which was purified by RP-HPLC to give the title compound (1.50 mg, 2% yield) as a yellow solid. [M+H]⁺=386.0. ¹H NMR (400 MHz, DMSO-d₆) δ ppm 13.99-13.55 (m, 1H), 10.25 (s, 1H), 9.18 (s, 1H), 8.35 (s, 1H), 8.31 (s, 1H), 8.25 (s, 1H), 7.28 (s, 1H), 3.95 (s, 3H), 1.33 (s, 9H).

### Example 12

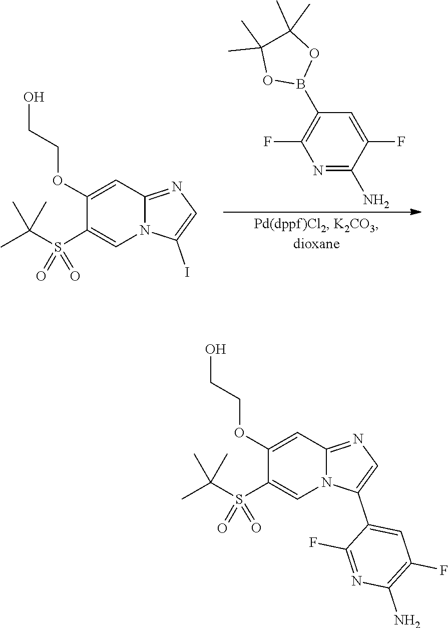 [SMILES: CC(C)(C)S(=O)(=O)c1cn2c(I)cnc2cc1OCCO ; CC1(C)OB(c2cc(F)c(N)nc2F)OC1(C)C ; CC(C)(C)S(=O)(=O)c1cn2c(-c3cc(F)c(N)nc3F)cnc2cc1OCCO]

2-((3-(6-amino-2,5-difluoropyridin-3-yl)-6-(tert-butylsulfonyl)imidazo[1,2-a]pyridin-7-yl)oxy)ethan-1-ol: A mixture of 2-((6-(tert-butylsulfonyl)-3-iodoimidazo[1,2-a]pyridin-7-yl)oxy)ethan-1-ol (25 mg, 53.03 μmol, 1 eq), 3,6-difluoro-5-(4,4,5,5-tetramethyl-1,3,2-dioxaborolan-2-yl)pyridin-2-amine (16.60 mg, 58.34 μmol, 1.1 eq), K₂CO₃ (21.99 mg, 159.10 μmol, 3 eq) and Pd(dppf)Cl₂ (3.88 mg, 5.30 μmol, 0.1 eq) in 1,4-dioxane (2 mL) and H₂O (0.4 mL) was degassed and purged with N₂ 3 times, and then the mixture was stirred at 80° C. for 4 hours under a N₂ atmosphere. The reaction mixture was diluted with water (3 mL) and extracted with EtOAc (3×3 mL).

The combined organic layers were washed with brine (3 mL), dried over Na₂SO₄, filtered and concentrated under reduced pressure to give a residue, which was purified by RP-HPLC to yield the title compound (5.2 mg, 22% yield) as a white solid. [M+H]⁺=427.0. ¹H NMR (400 MHz, DMSO-d₆) δ ppm 8.36 (d, J=2.4 Hz, 1H), 7.80-7.71 (m, 1H), 7.66 (s, 1H), 7.27 (s, 1H), 7.04 (s, 2H), 4.80 (t, J=5.2 Hz, 1H), 4.18 (t, J=4.8 Hz, 2H), 3.81-3.71 (m, 2H), 1.31 (s, 9H).

The following compounds were prepared following procedures analogous to that described for Example 12. Examples prepared using intermediates or building blocks containing Boc, THTP, TBS, dioxolane or other similar protecting groups were subjected to protecting group removal under acidic conditions (generally with HCl or TFA) prior to purification of the final product, as appropriate.

| Ex. | | | |
|---|---|---|---|
| No. | Structure | Name | LCMS, ¹H NMR |
| 13 | 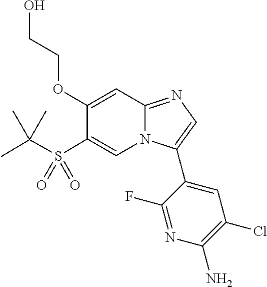 [SMILES: CC(C)(C)S(=O)(=O)c1cn2c(-c3cc(Cl)c(N)nc3F)cnc2cc1OCCO] | 2-((3-(6-amino-5- chloro-2- fluoropyridin-3-yl)-6- (tert- butylsulfonyl)imidazo [1,2-a]pyridin-7- yl)oxy)ethan-1-ol | [M + H]⁺ = 442.9 ¹H NMR (400 MHz, DMSO-d₆) δ ppm 8.36 (d, J = 2.0 Hz, 1H), 7.94 (d, J = 8.4 Hz, 1H), 7.68 (s, 1H), 7.28 (s, 1H), 7.12 (s, 2H), 4.81 (s, 1H), 4.18 (t, J = 4.8 Hz, 2H), 3.77 (s, 2H), 1.32 (s, 9H). |
| 14 | 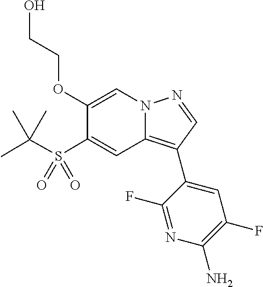 [SMILES: CC(C)(C)S(=O)(=O)c1cc2c(-c3cc(F)c(N)nc3F)cnn2cc1OCCO] | 2-((3-(6-amino-2,5- difluoropyridin-3-yl)- 5-(tert- butylsulfonyl)pyrazolo [1,5-a]pyridin-6- yl)oxy)ethan-1-ol | [M + H]⁺ = 427.1 ¹H NMR (400 MHz, DMSO-d₆) δ ppm 8.75 (s, 1H), 8.27 (s, 1H), 8.11 (t, J = 1.2 Hz, 1H), 7.87-7.76 (m, 1H), 6.80 (s, 2H), 4.82 (t, J = 5.6 Hz, 1H), 4.15 (t, J = 5.2 Hz, 2H), 3.75 (q, J = 5.2 Hz, 2H), 1.32 (s, 9H). |
| 15 | 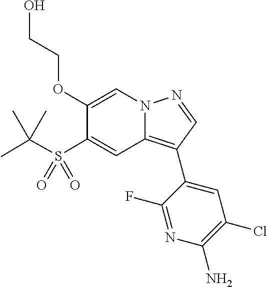 [SMILES: CC(C)(C)S(=O)(=O)c1cc2c(-c3cc(Cl)c(N)nc3F)cnn2cc1OCCO] | 2-((3-(6-amino-5- chloro-2- fluoropyridin-3-yl)-5- (tert- butylsulfonyl)pyrazolo [1,5-a]pyridin-6- yl)oxy)ethan-1-ol | [M + H]⁺ = 442.9 ¹H NMR (400 MHz, DMSO-d₆) δ ppm 8.75 (s, 1H), 8.29 (s, 1H), 8.09 (d, J = 1.6 Hz, 1H), 7.99-7.93 (m, 1H), 6.88 (s, 2H), 4.82 (t, J = 5.6 Hz, 1H), 4.15 (t, J = 5.2 Hz, 2H), 3.80-3.72 (m, 2H), 1.32 (s, 9H). |
| 16 | 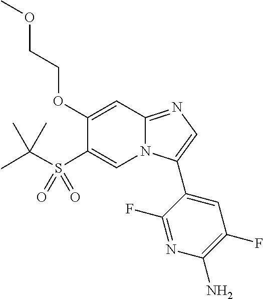 [SMILES: COCCOc1cc2ncc(-c3cc(F)c(N)nc3F)n2cc1S(=O)(=O)C(C)(C)C] | 5-(6-(tert- butylsulfonyl)-7-(2- methoxyethoxy)imidazo [1,2-a]pyridin-3-yl)- 3,6-difluoropyridin-2- amine | [M + H]⁺ = 441.2 ¹H NMR (400 MHz, DMSO-d₆) δ ppm 8.37 (d, J = 2.4 Hz, 1H), 7.82-7.70 (m, 1H), 7.67 (s, 1H), 7.28 (s, 1H), 7.06 (s, 2H), 4.33-4.23 (m, 2H), 3.76-3.66 (m, 2H), 3.32 (s, 3H), 1.31 (s, 9H). |
| 17 | 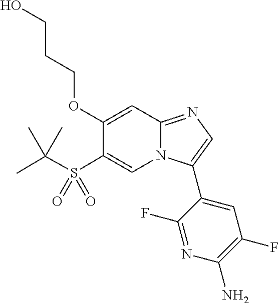 [SMILES: CC(C)(C)S(=O)(=O)c1cn2c(-c3cc(F)c(N)nc3F)cnc2cc1OCCCO] | 3-((3-(6-amino-2,5- difluoropyridin-3-yl)- 6-(tert- butylsulfonyl)imidazo [1,2-a]pyridin-7- yl)oxy)propan-1-ol | [M + H]⁺ = 441.0 ¹H NMR (400 MHz, DMSO-d₆) δ ppm 8.36 (d, J = 2.4 Hz, 1H), 7.77 (dd, J = 10.4, 7.2 Hz, 1H), 7.66 (s, 1H), 7.23 (s, 1H), 7.06 (s, 2H), 4.55 (t, J = 5.6 Hz, 1H), 4.20 (t, J = 5.4 Hz, 2H), 3.64-3.59 (m, 2H), 1.94- 1.87 (m, 2H), 1.31 (s, 9H). |
| 18 | 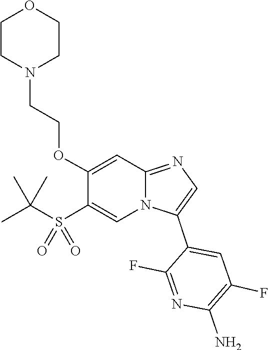 [SMILES: CC(C)(C)S(=O)(=O)c1cn2c(-c3cc(F)c(N)nc3F)cnc2cc1OCCN1CCOCC1] | 5-(6-(tert- butylsulfonyl)-7-(2- morpholinoethoxy) imidazo[1,2-a]pyridin-3- yl)-3,6- difluoropyridin-2- amine | [M + H]⁺ = 496.1 ¹H NMR (400 MHz, DMSO-d₆) δ ppm 8.36 (d, J = 2.4 Hz, 1H), 7.82-7.72 (s, 1H), 7.67 (s, 1H), 7.29 (s, 1H), 7.06 (s, 2H), 4.26 (t, J = 5.6 Hz, 2H), 3.65-3.53 (m, 4H), 3.35-3.29 (m, 4H), 2.77-2.68 (m, 2H), 1.32 (s, 9H). |
| 19 | 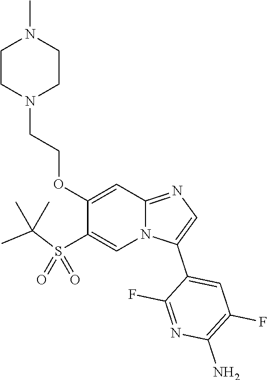 [SMILES: CN1CCN(CCOc2cc3ncc(-c4cc(F)c(N)nc4F)n3cc2S(=O)(=O)C(C)(C)C)CC1] | 5-(6-(tert- butylsulfonyl)-7-(2-(4- methylpiperazin-1- yl)ethoxy)imidazo[1,2- a]pyridin-3-yl)-3,6- difluoropyridin-2- amine | [M + H]⁺ = 509.1 ¹H NMR (400 MHz, DMSO-d₆) δ ppm 8.36 (d, J = 2.8 Hz, 1H), 7.81-7.72 (m, 1H), 7.66 (s, 1H), 7.28 (s, 1H), 7.06 (s, 2H), 4.24 (t, J = 5.6 Hz, 2H), 3.39-3.36 (m, 4H), 3.36-3.34 (m, 4H), 2.74-2.70 (m, 2H), 2.15 (s, 3H), 1.32 (s, 9H). |
| 20 | 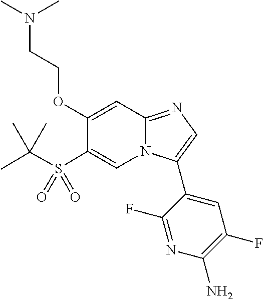 [SMILES: CN(C)CCOc1cc2ncc(-c3cc(F)c(N)nc3F)n2cc1S(=O)(=O)C(C)(C)C] | 5-(6-(tert- butylsulfonyl)-7-(2- (dimethylamino)ethoxy) imidazo[1,2- a]pyridin-3-yl)-3,6- difluoropyridin-2- amine | [M + H]⁺ = 454.1 ¹H NMR (400 MHz, DMSO-d₆) δ ppm 8.36 (d, J = 2.4 Hz, 1H), 7.82-7.70 (m, 1H), 7.67 (s, 1H), 7.30 (s, 1H), 7.05 (s, 2H), 4.22 (t, J = 6.0 Hz, 2H), 2.67 (t, J = 5.6 Hz, 2H), 2.24 (s, 6H), 1.31 (s, 9H). |
| 21 | 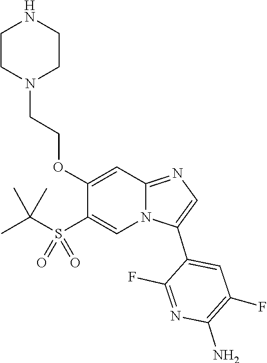 [SMILES: CC(C)(C)S(=O)(=O)c1cn2c(-c3cc(F)c(N)nc3F)cnc2cc1OCCN1CCNCC1] | 5-(6-(tert- butylsulfonyl)-7-(2- (piperazin-1- yl)ethoxy)imidazo[1,2- a]pyridin-3-yl)-3,6- difluoropyridin-2- amine | [M + H]⁺ = 495.1 ¹H NMR (400 MHz, DMSO-d₆) δ ppm 8.39- 8.32 (m, 2H), 7.80-7.71 (m, 1H), 7.67 (s, 1H), 7.29 (s, 1H), 7.06 (s, 2H), 4.25 (t, J = 4.8 Hz, 2H), 2.86-2.80 (m, 4H), 2.74 (t, J = 5.2 Hz, 2H), 2.58-2.49 (s, 4H), 1.32 (s, 9H). |
| 22 | 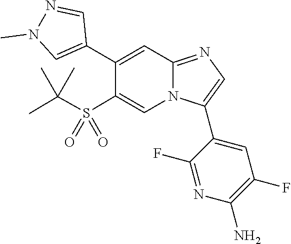 [SMILES: Cn1cc(-c2cc3ncc(-c4cc(F)c(N)nc4F)n3cc2S(=O)(=O)C(C)(C)C)cn1] | 5-(6-(tert- butylsulfonyl)-7-(1- methyl-1H-pyrazol-4- yl)imidazo[1,2- a]pyridin-3-yl)-3,6- difluoropyridin-2- amine | [M + H]⁺ = 446.9 ¹H NMR (400 MHz, DMSO-d₆) δ ppm 8.66- 8.60 (m, 1H), 7.99 (s, 1H), 7.88 (s, 1H), 7.82 (dd, J = 10.0, 7.6 Hz, 1H), 7.70 (s, 1H), 7.64 (s, 1H), 7.13 (s, 2H), 3.89 (s, 3H), 1.07 (s, 9H). |
| 23 | 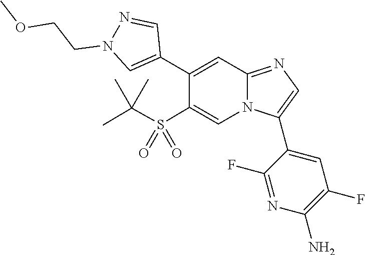 [SMILES: COCCn1cc(-c2cc3ncc(-c4cc(F)c(N)nc4F)n3cc2S(=O)(=O)C(C)(C)C)cn1] | 5-(6-(tert- butylsulfonyl)-7-(1-(2- methoxyethyl)-1H- pyrazol-4- yl)imidazo[1,2- a]pyridin-3-yl)-3,6- difluoropyridin-2- amine | [M + H]⁺ = 491.1 ¹H NMR (400 MHz, DMSO-d₆) δ ppm 8.64 (d, J = 2.0 Hz, 1H), 8.05 (s, 1H), 7.88 (s, 1H), 7.83 (dd, J = 10.4, 7.6 Hz, 1H), 7.73 (s, 1H), 7.66 (s, 1H), 7.13 (s, 2H), 4.30 (t, J = 5.2 Hz, 2H), 3.69 (t, J = 5.2 Hz, 2H), 3.24 (s, 3H), 1.06 (s, 9H). |
| 24 | 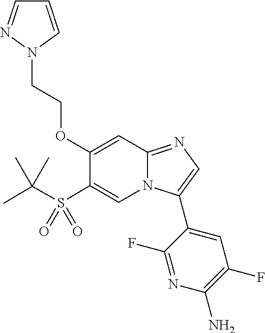 [SMILES: CC(C)(C)S(=O)(=O)c1cn2c(-c3cc(F)c(N)nc3F)cnc2cc1OCCn1cccn1] | 5-(7-(2-(1H-pyrazol-1- yl)ethoxy)-6-(tert- butylsulfonyl)imidazo [1,2-a]pyridin-3-yl)- 3,6-difluoropyridin-2- amine | [M + H]⁺ = 477.0 ¹H NMR (400 MHz, DMSO-d₆) δ ppm 8.35 (s, 1H), 7.88 (d, J = 2.0 Hz, 1H), 7.87-7.62 (m, 1H), 7.67 (s, 1H), 7.46 (d, J = 1.6 Hz, 1H), 7.30 (s, 1H), 7.06 (s, 2H), 6.25 (t, J = 2.0 Hz, 1H), 4.54 (s, 4H), 1.20 (s, 9H). |
| 25 | 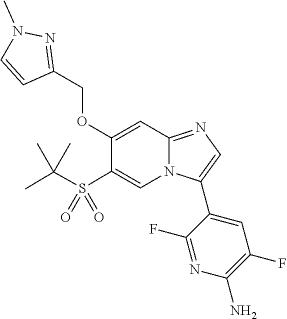 [SMILES: Cn1ccc(COc2cc3ncc(-c4cc(F)c(N)nc4F)n3cc2S(=O)(=O)C(C)(C)C)n1] | 5-(6-(tert- butylsulfonyl)-7-((1- methyl-1H-pyrazol-3- yl)methoxy)imidazo[1, 2-a]pyridin-3-yl)-3,6- difluoropyridin-2- amine | [M + H]⁺ = 477.0 ¹H NMR (400 MHz, DMSO-d₆) δ ppm 8.37 (d, J = 2.4 Hz, 1H), 8.03-7.71 (m, 1H), 7.69 (d, J = 2.0 Hz, 1H), 7.67 (s, 1H), 7.43 (s, 1H), 7.06 (s, 2H), 6.36 (d, J = 2.4 Hz, 1H), 5.20 (s, 2H), 3.84 (s, 3H), 1.27 (s, 9H). |
| 26 | 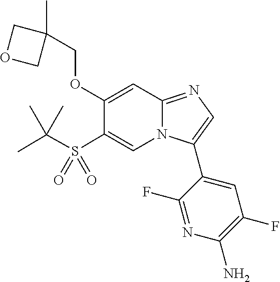 [SMILES: CC1(COc2cc3ncc(-c4cc(F)c(N)nc4F)n3cc2S(=O)(=O)C(C)(C)C)COC1] | 5-(6-(tert- butylsulfonyl)-7-((3- methyloxetan-3- yl)methoxy)imidazo[1, 2-a]pyridin-3-yl)-3,6- difluoropyridin-2- amine | [M + H]⁺ = 467.1 ¹H NMR (400 MHz, DMSO-d₆) δ ppm 8.37 (d, J = 2.4 Hz, 1H), 7.82-7.71 (m, 1H), 7.68 (s, 1H), 7.33 (s, 1H), 7.07 (s, 2H), 4.52 (d, J = 5.6 Hz, 2H), 4.31 (d, J = 5.6 Hz, 2H), 4.27 (s, 2H), 1.43 (s, 3H), 1.30 (s, 9H). |
| 27 | 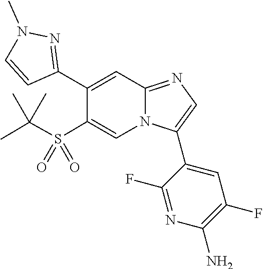 [SMILES: Cn1ccc(-c2cc3ncc(-c4cc(F)c(N)nc4F)n3cc2S(=O)(=O)C(C)(C)C)n1] | 5-(6-(tert- butylsulfonyl)-7-(1- methyl-1H-pyrazol-3- yl)imidazo[1,2- a]pyridin-3-yl)-3,6- difluoropyridin-2- amine | [M + H]⁺ = 447.1 ¹H NMR (400 MHz, DMSO-d₆) δ ppm 8.79 (s, 1H), 8.40 (s, 1H), 8.05 (s, 1H), 7.84 (dd, J = 10.0, 7.2 Hz, 1H), 7.81 (d, J = 2.0 Hz, 1H), 7.72-6.86 (m, 2H), 6.76 (d, J = 2.4 Hz, 1H), 3.95 (s, 3H), 1.10 (s, 9H). |
| 28 | 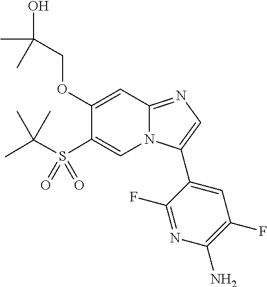 [SMILES: CC(C)(O)COc1cc2ncc(-c3cc(F)c(N)nc3F)n2cc1S(=O)(=O)C(C)(C)C] | 1-((3-(6-amino-2,5- difluoropyridin-3-yl)- 6-(tert- butylsulfonyl)imidazo [1,2-a]pyridin-7- yl)oxy)-2- methylpropan-2-ol | [M + H]⁺ = 455.1 ¹H NMR (400 MHz, DMSO-d₆) δ ppm 8.36 (d, J = 2.4 Hz, 1H), 7.76 (dd, J = 10.4, 7.2 Hz, 1H), 7.66 (s, 1H), 7.24 (s, 1H), 7.06 (s, 2H), 4.53 (s, 1H), 3.90 (s, 2H), 1.32 (s, 9H), 1.26 (s, 6H). |
| 29 | 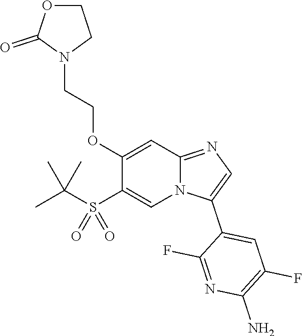 [SMILES: CC(C)(C)S(=O)(=O)c1cn2c(-c3cc(F)c(N)nc3F)cnc2cc1OCCN1CCOC1=O] | 3-(2-((3-(6-amino-2,5- difluoropyridin-3-yl)- 6-(tert- butylsulfonyl)imidazo [1,2-a]pyridin-7- yl)oxy)ethyl)oxazolidin- 2-one | [M + H]⁺ = 496.1 ¹H NMR (400 MHz, DMSO-d₆) δ ppm 8.37 (d, J = 2.0 Hz, 1H), 7.77 (dd, J = 10.4, 7.6 Hz, 1H), 7.68 (s, 1H), 7.33 (s, 1H), 7.06 (s, 2H), 4.32 (t, J = 5.2 Hz, 2H), 4.28-4.23 (m, 2H), 3.77- 3.67 (m, 2H), 3.59 (t, J = 5.2 Hz, 2H), 1.31 (s, 9H). |
| 30 | 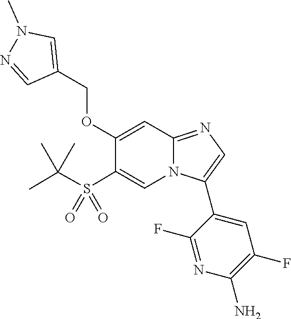 [SMILES: Cn1cc(COc2cc3ncc(-c4cc(F)c(N)nc4F)n3cc2S(=O)(=O)C(C)(C)C)cn1] | 5-(6-(tert- butylsulfonyl)-7-((1- methyl-1H-pyrazol-4- yl)methoxy)imidazo[1, 2-a]pyridin-3-yl)-3,6- difluoropyridin-2- amine | [M + H]⁺ = 477.1 ¹H NMR (400 MHz, DMSO-d₆) δ ppm 8.37 (d, J = 2.4 Hz, 1H), 7.81 (s, 1H), 7.77 (dd, J = 10.0, 7.6 Hz, 1H), 7.67 (s, 1H), 7.52 (s, 1H), 7.39 (s, 1H), 7.07 (s, 2H), 5.15 (s, 2H), 3.84 (s, 3H), 1.26 (s, 9H). |
| 31 | 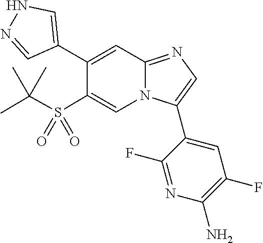 [SMILES: CC(C)(C)S(=O)(=O)c1cn2c(-c3cc(F)c(N)nc3F)cnc2cc1-c1cn[nH]c1] | 5-(6-(tert- butylsulfonyl)-7-(1H- pyrazol-4- yl)imidazo[1,2- a]pyridin-3-yl)-3,6- difluoropyridin-2- amine | [M + H]⁺ = 433.0 ¹H NMR (400 MHz, DMSO-d₆) δ ppm 13.00 (s, 1H), 8.64 (d, J = 2.0 Hz, 1H), 8.02 (s, 1H), 7.88 (s, 1H), 7.86-7.79 (m, 1H), 7.77 (s, 1H), 7.67 (s, 1H), 7.13 (s, 2H), 1.05 (s, 9H). |
| 32 | 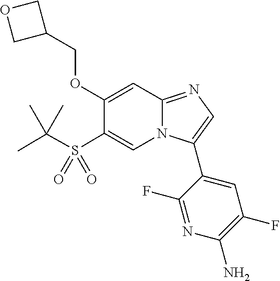 [SMILES: CC(C)(C)S(=O)(=O)c1cn2c(-c3cc(F)c(N)nc3F)cnc2cc1OCC1COC1] | 5-(6-(tert- butylsulfonyl)-7- (oxetan-3- ylmethoxy)imidazo[1, 2-a]pyridin-3-yl)-3,6- difluoropyridin-2- amine | [M + H]⁺ = 453.1 ¹H NMR (400 MHz, DMSO-d₆) δ ppm 8.37 (d, J = 2.4 Hz, 1H), 7.81-7.70 (m, 1H), 7.68 (s, 1H), 7.32 (s, 1H), 7.07 (s, 2H), 4.75-4.66 (m, 2H), 4.48 (t, J = 6.0 Hz, 2H), 4.39 (d, J = 6.4 Hz, 2H), 3.50-3.38 (m, 1H), 1.30 (s, 9H). |
| 33 | 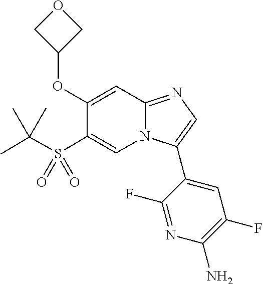 [SMILES: CC(C)(C)S(=O)(=O)c1cn2c(-c3cc(F)c(N)nc3F)cnc2cc1OC1COC1] | 5-(6-(tert- butylsulfonyl)-7- (oxetan-3- yloxy)imidazo[1,2- a]pyridin-3-yl)-3,6- difluoropyridin-2- amine | [M + H]⁺ = 439.0 ¹H NMR (400 MHz, DMSO-d₆) δ ppm 8.40 (d, J = 2.4 Hz, 1H), 7.80-7.71 (m, 1H), 7.68 (s, 1H), 7.07 (s, 2H), 6.93 (s, 1H), 5.52-5.42 (m, 1H), 5.02 (t, J = 6.8 Hz, 2H), 4.63-4.55 (m, 2H), 1.36 (s, 9H). |
| 34 | 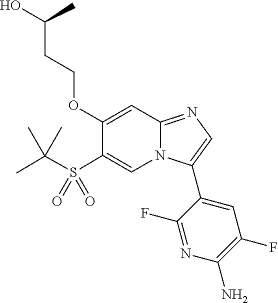 [SMILES: C[C@H](O)CCOc1cc2ncc(-c3cc(F)c(N)nc3F)n2cc1S(=O)(=O)C(C)(C)C] | (S)-4-((3-(6-amino- 2,5-difluoropyridin-3- yl)-6-(tert- butylsulfonyl)imidazo [1,2-a]pyridin-7- yl)oxy)butan-2-ol | [M + H]⁺ = 455.0 ¹H NMR (400 MHz, DMSO-d₆) δ ppm 8.36 (d, J = 2.4 Hz, 1H), 7.80-7.72 (m, 1H), 7.66 (s, 1H), 7.24 (s, 1H), 7.06 (s, 2H), 4.57 (d, J = 4.4 Hz, 1H), 4.29-4.12 (m, 2H), 3.93-3.85 (m, 1H), 1.90-1.72 (m, 2H), 1.31 (s, 9H), 1.13 (d, J = 6.0 Hz, 3H). SFC: Rₜ = 0.992 min, 89% ee; CHIRALCEL OJ-3 50 x 4.6 mm, 3 μm; 5-40% MeOH (0.05% DEA) in CO₂, 3 mL/min; 35° C., 100 Bar, 220 nm |
| 35 | 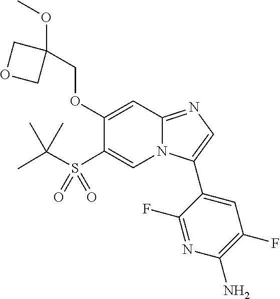 [SMILES: COC1(COc2cc3ncc(-c4cc(F)c(N)nc4F)n3cc2S(=O)(=O)C(C)(C)C)COC1] | 5-(6-(tert- butylsulfonyl)-7-((3- methoxyoxetan-3- yl)methoxy)imidazo[1, 2-a]pyridin-3-yl)-3,6- difluoropyridin-2- amine | [M + H]⁺ = 483.0 ¹H NMR (400 MHz, DMSO-d₆) δ ppm 8.38 (d, J = 2.8 Hz, 1H), 7.78 (dd, J = 10.0, 7.2 Hz, 1H), 7.69 (s, 1H), 7.40 (s, 1H), 7.07 (s, 2H), 4.62-4.58 (m, 2H), 4.56- 4.52 (m, 2H), 4.49 (s, 2H), 3.29 (s, 3H), 1.31 (s, 9H). |
| 36 | 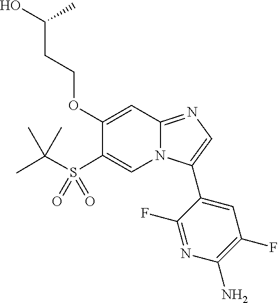 [SMILES: C[C@@H](O)CCOc1cc2ncc(-c3cc(F)c(N)nc3F)n2cc1S(=O)(=O)C(C)(C)C] | (R)-4-((3-(6-amino- 2,5-difluoropyridin-3- yl)-6-(tert- butylsulfonyl)imidazo [1,2-a]pyridin-7- yl)oxy)butan-2-ol | [M + H]⁺ = 455.1 ¹H NMR (400 MHz, CDCl₃) δ ppm 8.36 (d, J = 2.4 Hz, 1H), 7.80-7.73 (m, 1H), 7.66 (s, 1H), 7.24 (s, 1H), 7.06 (s, 2H), 4.60-4.52 (m, 1H), 4.28-4.13 (m, 2H), 3.94- 3.82 (m, 1H), 1.86-1.75 (m, 2H), 1.31 (s, 9H), 1.13 (d, J = 6.0 Hz, 3H). SFC: Rₜ = 2.575 min, 85% ee; CHIRALPAK IK-3 50 x 4.6 mm, 3 μm; 5-40% MeOH (0.05% DEA) in CO₂, 3 mL/min; 35° C., 100 Bar, 220 nm |
| 37 | 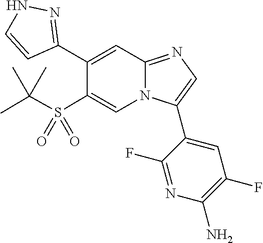 [SMILES: CC(C)(C)S(=O)(=O)c1cn2c(-c3cc(F)c(N)nc3F)cnc2cc1-c1cc[nH]n1] | 5-(6-(tert- butylsulfonyl)-7-(1H- pyrazol-3- yl)imidazo[1,2- a]pyridin-3-yl)-3,6- difluoropyridin-2- amine | [M + H]⁺ = 433.0 ¹H NMR (400 MHz, DMSO-d₆) δ ppm 13.02 (br s, 1H), 8.65 (d, J = 1.6 Hz, 1H), 7.91 (s, 1H), 7.85 (dd, J = 10.4, 7.6 Hz, 1H), 7.77 (s, 1H), 7.70 (s, 1H), 7.14 (s, 2H), 6.78-6.55 (m, 1H), 1.06 (s, 9H). |
| 38 | 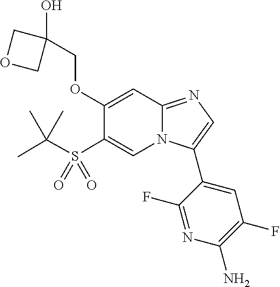 [SMILES: CC(C)(C)S(=O)(=O)c1cn2c(-c3cc(F)c(N)nc3F)cnc2cc1OCC1(O)COC1] | 3-(((3-(6-amino-2,5- difluoropyridin-3-yl)- 6-(tert- butylsulfonyl)imidazo [1,2-a]pyridin-7- yl)oxy)methyl)oxetan- 3-ol | [M + H]⁺ = 469.0 ¹H NMR (400 MHz, DMSO-d₆) δ ppm 8.38 (d, J = 2.4 Hz, 1H), 8.01-7.73 (m, 1H), 7.68 (s, 1H), 7.35 (s, 1H), 7.07 (s, 2H), 6.00 (s, 1H), 4.59 (d, J = 6.4 Hz, 2H), 4.49 (d, J = 6.8 Hz, 2H), 4.30 (s, 2H), 1.32 (s, 9H). |
| 39 | 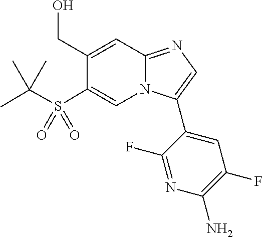 [SMILES: CC(C)(C)S(=O)(=O)c1cn2c(-c3cc(F)c(N)nc3F)cnc2cc1CO] | (3-(6-amino-2,5- difluoropyridin-3-yl)- 6-(tert- butylsulfonyl)imidazo [1,2-a]pyridin-7- yl)methanol | [M + H]⁺ = 397.0 ¹H NMR (400 MHz, DMSO-d₆) δ ppm 8.39 (d, J = 1.6 Hz, 1H), 7.90 (s, 1H), 7.83 (s, 1H), 7.82-7.74 (m, 1H), 7.11 (s, 2H), 5.69-5.57 (m, 1H), 4.89-4.84 (m, 2H), 1.30 (s, 9H). |
| 40 | 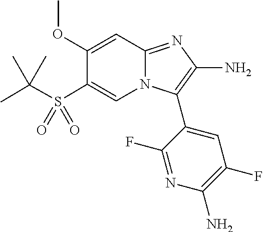 [SMILES: COc1cc2nc(N)c(-c3cc(F)c(N)nc3F)n2cc1S(=O)(=O)C(C)(C)C] | 3-(6-amino-2,5- difluoropyridin-3-yl)- 6-(tert-butylsulfonyl)- 7- methoxyimidazo[1,2- a]pyridin-2-amine | [M + H]⁺ = 412.0 ¹H NMR (400 MHz, DMSO-d₆) δ ppm 7.94 (d, J = 2.4 Hz, 1H), 7.60 (dd, J = 10.4, 7.6 Hz, 1H), 6.93 (s, 1H), 6.88 (s, 2H), 5.47 (s, 2H), 3.87 (s, 3H), 1.26 (s, 9H). |
| 41 | 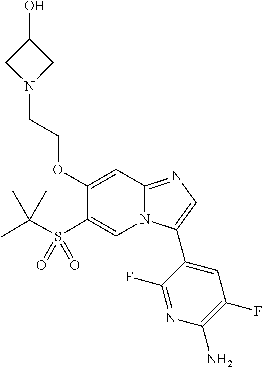 [SMILES: CC(C)(C)S(=O)(=O)c1cn2c(-c3cc(F)c(N)nc3F)cnc2cc1OCCN1CC(O)C1] | 1-(2-((3-(6-amino-2,5- difluoropyridin-3-yl)- 6-(tert- butylsulfonyl)imidazo [1,2-a]pyridin-7- yl)oxy)ethyl)azetidin- 3-ol | [M + H]⁺ = 481.9 ¹H NMR (400 MHz, DMSO-d₆) δ ppm 8.36 (d, J = 2.8 Hz, 1H), 7.82-7.71 (m, 1H), 7.67 (s, 1H), 7.25 (s, 1H), 7.07 (s, 2H), 5.27 (d, J = 6.4 Hz, 1H), 4.16-4.10 (m, 1H), 4.09-4.05 (m, 2H), 3.62-3.54 (m, 2H), 2.87-2.74 (m, 4H), 1.31 (s, 9H). |
| 42 | 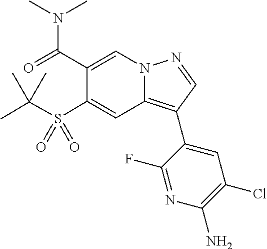 | 3-(6-amino-2,5- difluoropyridin-3-yl)- 6-(tert-butylsulfonyl)- N,N- dimethylimidazo[1,2- a]pyridine-7- carboxamide | [M + H]⁺ = 437.8 ¹H NMR (400 MHz, DMSO-d₆) δ ppm 8.49 (s, 1H), 7.92 (s, 1H), 7.85-7.76 (m, 1H), 7.68 (s, 1H), 7.13 (s, 2H), 2.96 (s, 3H), 2.71 (s, 3H), 1.31 (s, 9H). |
| 43 | 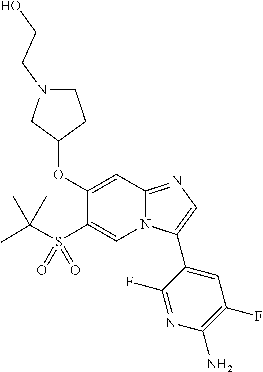 [SMILES: CC(C)(C)S(=O)(=O)c1cn2c(-c3cc(F)c(N)nc3F)cnc2cc1OC1CCN(CCO)C1] | 2-(3-((3-(6-amino-2,5- difluoropyridin-3-yl)- 6-(tert- butylsulfonyl)imidazo [1,2-a]pyridin-7- yl)oxy)pyrrolidin-1- yl)ethan-1-ol | [M + H]⁺ = 496.1 ¹H NMR (400 MHz, DMSO-d₆) δ ppm 8.36 (d, J = 2.4 Hz, 1H), 7.81-7.70 (m, 1H), 7.66 (s, 1H), 7.19-7.08 (m, 1H), 7.05 (s, 2H), 5.11- 4.98 (m, 1H), 4.49-4.29 (m, 1H), 3.53-3.44 (m, 2H), 3.09-3.04 (m, 1H), 2.71-2.54 (m, 3H), 2.37- 2.24 (m, 3H), 1.90-1.82 (m, 1H), 1.31 (s, 9H). |
| 44 | 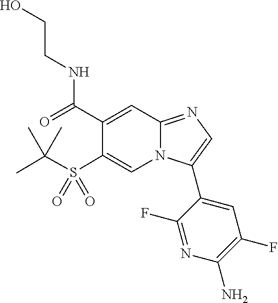 [SMILES: CC(C)(C)S(=O)(=O)c1cn2c(-c3cc(F)c(N)nc3F)cnc2cc1C(=O)NCCO] | 3-(6-amino-2,5- difluoropyridin-3-yl)- 6-(tert-butylsulfonyl)- N-(2- hydroxyethyl)imidazo [1,2-a]pyridine-7- carboxamide | [M + H]⁺ = 454.1 ¹H NMR (400 MHz, DMSO-d₆) δ ppm 8.44 (d, J = 2.0 Hz, 1H), 8.36 (t, J = 5.6 Hz, 1H), 7.93 (s, 1H), 7.85-7.77 (m, 1H), 7.75 (s, 1H), 7.14 (s, 2H), 4.65 (t, J = 5.6 Hz, 1H), 3.57-3.46 (m, 2H), 3.32-3.21 (m, 2H), 1.33 (s, 9H). |
| 45 | 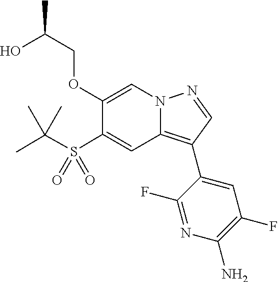 [SMILES: C[C@H](O)COc1cn2ncc(-c3cc(F)c(N)nc3F)c2cc1S(=O)(=O)C(C)(C)C] | (S)-1-((3-(6-amino- 2,5-difluoropyridin-3- yl)-5-(tert- butylsulfonyl)pyrazolo [1,5-a]pyridin-6- yl)oxy)propan-2-ol | [M + H]⁺ = 441.0 ¹H NMR (400 MHz, DMSO-d₆) δ ppm 8.72 (s, 1H), 8.26 (s, 1H), 8.10 (d, J = 1.2 Hz, 1H), 7.81 (d, J = 7.6 Hz, 1H), 6.79 (s, 2H), 4.81-4.78 (m, 1H), 4.04-3.93 (m, 3H), 1.32 (s, 9H), 1.22- 1.19 (m, 1H). SFC: Rₜ = 1.608 min, 100% ee; CHIRALPAK AD-3 50 x 4.6 mm, 3 μm; 5-40% MeOH (0.05% DEA) in CO₂, 3 mL/min; 35° C., 100 Bar, 220 nm |
| 46 | 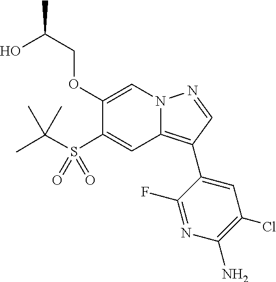 [SMILES: C[C@H](O)COc1cn2ncc(-c3cc(Cl)c(N)nc3F)c2cc1S(=O)(=O)C(C)(C)C] | (S)-1-((3-(6-amino-5- chloro-2- fluoropyridin-3-yl)-5- (tert- butylsulfonyl)pyrazolo [1,5-a]pyridin-6- yl)oxy)propan-2-ol | [M + H]⁺ = 456.9 ¹H NMR (400 MHz, DMSO-d₆) δ ppm 8.72 (s, 1H), 8.29 (s, 1H), 8.09 (d, J = 1.6 Hz, 1H), 7.97 (d, J = 8.4 Hz, 1H), 6.88 (s, 2H), 4.80 (d, J = 4.0 Hz, 1H), 4.02-3.93 (m, 3H), 1.32 (s, 9H), 1.21 (d, J = 6.0 Hz, 3H). SFC: Rₜ = 1.355 min, 100% ee; CHIRALCEL OJ-3 50 x 4.6 mm, 3 μm; 5-40% MeOH (0.05% DEA) in CO₂, 3 mL/min; 35° C., 100 Bar, 220 nm |
| 47 | 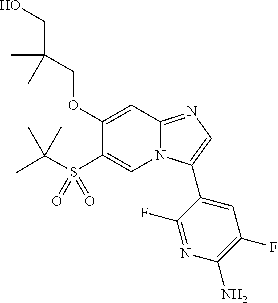 [SMILES: CC(C)(CO)COc1cc2ncc(-c3cc(F)c(N)nc3F)n2cc1S(=O)(=O)C(C)(C)C] | 3-((3-(6-amino-2,5- difluoropyridin-3-yl)- 6-(tert- butylsulfonyl)imidazo [1,2-a]pyridin-7- yl)oxy)-2,2- dimethylpropan-1-ol | [M + H]⁺ = 469.1 ¹H NMR (400 MHz, DMSO-d₆) δ ppm 8.35 (d, J = 2.0 Hz, 1H), 7.76 (dd, J = 10.4, 7.6 Hz, 1H), 7.66 (s, 1H), 7.22 (s, 1H), 7.05 (s, 2H), 4.57 (t, J = 5.6 Hz, 1H), 3.89 (s, 2H), 3.33 (s, 2H), 1.31 (s, 9H), 0.97 (s, 6H). |
| 48 | 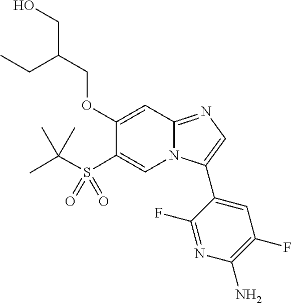 [SMILES: CCC(CO)COc1cc2ncc(-c3cc(F)c(N)nc3F)n2cc1S(=O)(=O)C(C)(C)C] | 2-(((3-(6-amino-2,5- difluoropyridin-3-yl)- 6-(tert- butylsulfonyl)imidazo [1,2-a]pyridin-7- yl)oxy)methyl)butan- 1-ol | [M + H]⁺ = 469.1 ¹H NMR (400 MHz, DMSO-d₆) δ ppm 8.36 (d, J = 2.4 Hz, 1H), 7.76 (dd, J = 10.4, 7.6 Hz, 1H), 7.66 (s, 1H), 7.26 (s, 1H), 7.05 (s, 2H), 4.51 (t, J = 5.6 Hz, 1H), 4.11 (d, J = 5.6 Hz, 2H), 3.55-3.50 (m, 2H), 1.82- 1.76 (m, 1H), 1.50-1.43 (m, 2H), 1.30 (s, 9H), 0.92 (t, J = 7.2 Hz, 3H). |
| 49 | 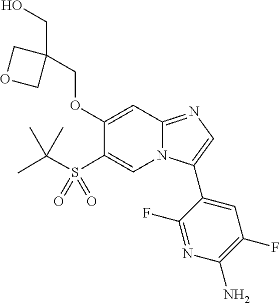 [SMILES: CC(C)(C)S(=O)(=O)c1cn2c(-c3cc(F)c(N)nc3F)cnc2cc1OCC1(CO)COC1] | (3-(((3-(6-amino-2,5- difluoropyridin-3-yl)- 6-(tert- butylsulfonyl)imidazo [1,2-a]pyridin-7- yl)oxy)methyl)oxetan- 3-yl)methanol | [M + H]⁺ = 483.0 ¹H NMR (400 MHz, DMSO-d₆) δ ppm 8.37 (d, J = 2.4 Hz, 1H), 7.77 (dd, J = 10.0, 7.2 Hz, 1H), 7.68 (s, 1H), 7.33 (s, 1H), 7.06 (s, 2H), 4.98 (t, J = 5.2 Hz, 1H), 4.47-4.40 (m, 4H), 4.35 (s, 2H), 3.77 (d, J = 5.2 Hz, 2H), 1.29 (s, 9H). |
| 50 | 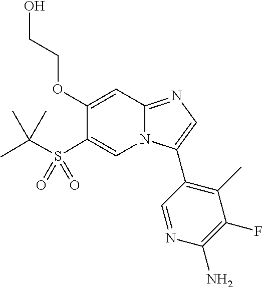 [SMILES: Cc1c(-c2cnc3cc(OCCO)c(S(=O)(=O)C(C)(C)C)cn23)cnc(N)c1F] | 2-((3-(6-amino-5- fluoro-4- methylpyridin-3-yl)-6- (tert- butylsulfonyl)imidazo [1,2-a]pyridin-7- yl)oxy)ethan-1-ol | [M + H]⁺ = 423.1 ¹H NMR (400 MHz, DMSO-d₆) δ ppm 8.18 (s, 1H), 7.78 (s, 1H), 7.61 (s, 1H), 7.28 (s, 1H), 6.52 (s, 2H), 4.80 (t, J = 5.2 Hz, 1H), 4.18 (t, J = 5.2 Hz, 2H), 3.81-3.72 (m 2H), 1.96 (d, J = 2.0 Hz, 3H), 1.30 (s, 9H). |
| 51 | 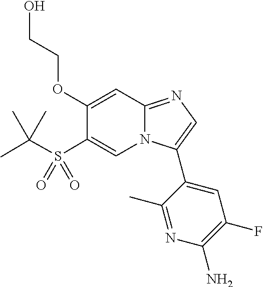 [SMILES: Cc1nc(N)c(F)cc1-c1cnc2cc(OCCO)c(S(=O)(=O)C(C)(C)C)cn12] | 2-((3-(6-amino-5- fluoro-2- methylpyridin-3-yl)-6- (tert- butylsulfonyl)imidazo [1,2-a]pyridin-7- yl)oxy)ethan-1-ol | [M + H]⁺ = 423.1 ¹H NMR (400 MHz, DMSO-d₆) δ ppm 8.13 (s, 1H), 7.59 (s, 1H), 7.43 (d, J = 11.6 Hz, 1H), 7.27 (s, 1H), 6.57 (s, 2H), 4.80 (t, J = 5.2 Hz, 1H), 4.18 (t, J = 5.2 Hz, 2H), 3.79-3.75 (m, 2H), 2.05 (s, 3H), 1.31 (s, 9H). |
| 52 | 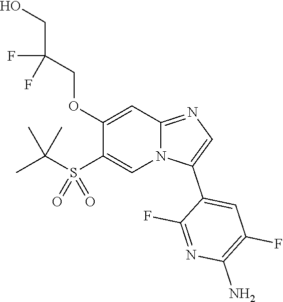 | 2-((6-(tert- butylsulfonyl)-3-(6- fluoro-1H-pyrrolo[2,3- b]pyridin-5- yl)imidazo[1,2- a]pyridin-7- yl)oxy)ethan-1-ol | [M + H]⁺ = 433.0 ¹H NMR (400 MHz, DMSO-d₆) δ ppm 12.08 (s, 1H), 8.39 (d, J = 2.8 Hz, 1H), 8.31 (d, J = 9.6 Hz, 1H), 7.77 (s, 1H), 7.57 (dd, J = 3.2, 2.8 Hz, 1H), 7.32 (s, 1H), 6.61 (dd, J = 3.2, 1.6 Hz, 1H), 4.82 (s, 1H), 4.20 (t, J = 5.2 Hz, 2H), 3.78 (s, 2H), 1.31 (s, 9H). |
| 53 | 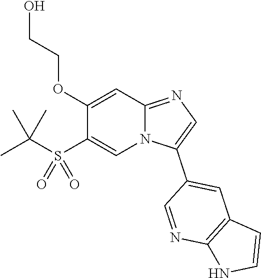 [SMILES: CC(C)(C)S(=O)(=O)c1cn2c(-c3cnc4[nH]ccc4c3)cnc2cc1OCCO] | 2-((6-(tert- butylsulfonyl)-3-(1H- pyrrolo[2,3-b]pyridin- 5-yl)imidazo[1,2- a]pyridin-7- yl)oxy)ethan-1-ol | [M + H]⁺ = 415.0 ¹H NMR (400 MHz, DMSO-d₆) δ ppm 11.94 (s, 1H), 8.63 (s, 1H), 8.41 (d, J = 2.0 Hz, 1H), 8.19 (d, J = 2.0 Hz, 1H), 7.74 (s, 1H), 7.61 (t, J = 2.8 Hz, 1H), 7.29 (s, 1H), 6.59-6.54 (m, 1H), 4.88-3.77 (m, 1H), 4.19 (t, J = 4.8 Hz, 2H), 3.82-7.78 (m, 2H), 1.31 (s, 9H) |
| 54 | 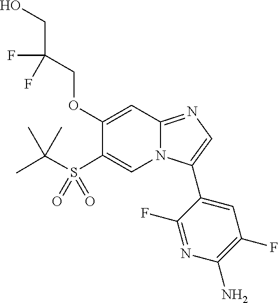 [SMILES: CC(C)(C)S(=O)(=O)c1cn2c(-c3cc(F)c(N)nc3F)cnc2cc1OCC(F)(F)CO] | 3-((3-(6-amino-2,5- difluoropyridin-3-yl)- 6-(tert- butylsulfonyl)imidazo [1,2-a]pyridin-7- yl)oxy)-2,2- difluoropropan-1-ol | [M + H]⁺ = 477.0 ¹H NMR (400 MHz, DMSO-d₆) δ ppm 8.39 (d, J = 2.4 Hz, 1H), 7.77 (dd, J = 10.4, 7.6 Hz, 1H), 7.70 (s, 1H), 7.41 (s, 1H), 7.07 (s, 2H), 5.61 (t, J = 6.4 Hz, 1H), 4.49 (t, J = 12.0 Hz, 2H), 3.90-3.80 (m, 2H), 1.30 (s, 9H). |
| 55 | 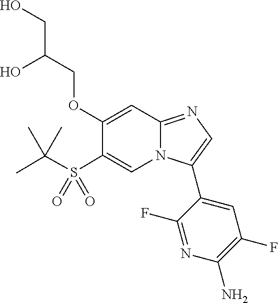 [SMILES: CC(C)(C)S(=O)(=O)c1cn2c(-c3cc(F)c(N)nc3F)cnc2cc1OCC(O)CO] | 3-((3-(6-amino-2,5- difluoropyridin-3-yl)- 6-(tert- butylsulfonyl)imidazo [1,2-a]pyridin-7- yl)oxy)propane-1,2- diol | [M + H]⁺ = 457.0 ¹H NMR (400 MHz, DMSO-d₆) δ ppm 8.36 (d, J = 2.0 Hz, 1H), 7.77 (dd, J = 10.0, 7.4 Hz, 1H), 7.66 (s, 1H), 7.25 (s, 1H), 7.05 (s, 2H), 4.89 (d, J = 4.8 Hz, 1H), 4.66 (t, J = 5.6 Hz, 1H), 4.17-4.12 (m, 1H), 4.11- 4.05 (m, 1H), 3.90-3.80 (m, 1H), 3.57-3.46 (m, 2H), 1.31 (s, 9H). |
| 56 | 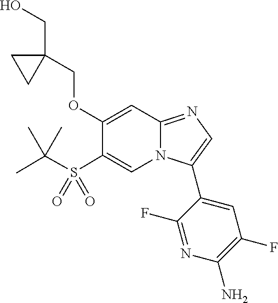 [SMILES: CC(C)(C)S(=O)(=O)c1cn2c(-c3cc(F)c(N)nc3F)cnc2cc1OCC1(CO)CC1] | (1-(((3-(6-amino-2,5- difluoropyridin-3-yl)- 6-(tert- butylsulfonyl)imidazo [1,2-a]pyridin-7- yl)oxy)methyl) cyclopropyl)methanol | [M + H]⁺ = 467.1 |
| 57 | 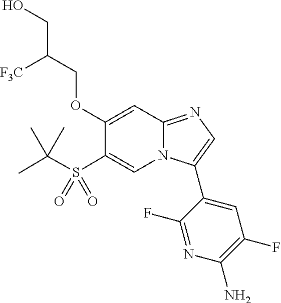 [SMILES: CC(C)(C)S(=O)(=O)c1cn2c(-c3cc(F)c(N)nc3F)cnc2cc1OCC(CO)C(F)(F)F] | 2-(((3-(6-amino-2,5- difluoropyridin-3-yl)- 6-(tert- butylsulfonyl)imidazo [1,2-a]pyridin-7- yl)oxy)methyl)-3,3,3- trifluoropropan-1-ol | [M + H]⁺ = 509.0 |
| 58 | 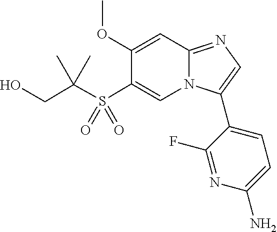 | 2-((3-(6-amino-2- fluoropyridin-3-yl)-7- methoxyimidazo[1,2- a]pyridin-6- yl)sulfonyl)-2- methylpropan-1-ol | [M + H]⁺ = 497.6 ¹H NMR (400 MHz, DMSO-d₆) δ ppm 8.30 (d, J = 2.0 Hz, 1H), 7.68-7.60 (m, 2H), 7.21(s, 1H), 6.77 (s, 2H), 6.48 (dd, J = 2.0 Hz, 1H), 4.96 (s, 1H), 3.92 (s, 3H), 3.54 (s, 2H), 1.25 (s, 6H). |
| 59 | 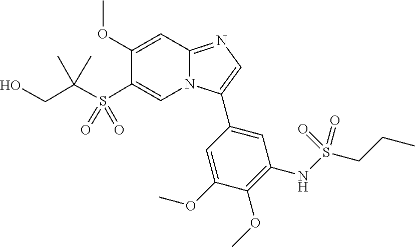 [SMILES: CCCS(=O)(=O)Nc1cc(-c2cnc3cc(OC)c(S(=O)(=O)C(C)(C)CO)cn23)cc(OC)c1OC] | N-(5-(6-((1-hydroxy- 2-methylpropan-2- yl)sulfonyl)-7- methoxyimidazo[1,2- a]pyridin-3-yl)-2,3- dimethoxyphenyl) propane-1-sulfonamide | [M + H]⁺ = 542.3 ¹H NMR (400 MHz, DMSO-d₆) δ ppm 9.21 (br s, 1H), 8.79 (s, 1H), 7.68 (s, 1H), 7.21 (s, 1H), 7.17 (d, J = 1.60 Hz, 1H), 6.88 (br s, 1H), 4.98 (t, J = 5.60 Hz, 1H), 3.93 (s, 3H), 3.83 (s, 3H), 3.78 (s, 3H), 3.58 (d, J = 5.60 Hz, 2H), 3.02 (br s, 2H), 1.72 (q, J = 7.60 Hz, 2H), 1.28 (s, 6H), 0.95 (t, J = 7.60 Hz, 3H) |
| 60 | 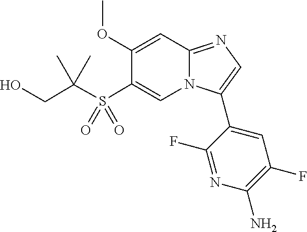 [SMILES: COc1cc2ncc(-c3cc(F)c(N)nc3F)n2cc1S(=O)(=O)C(C)(C)CO] | 2-((3-(6-amino-2,5- difluoropyridin-3-yl)- 7- methoxyimidazo[1,2- a]pyridin-6- yl)sulfonyl)-2- methylpropan-1-ol | [M + H]⁺ = 412.9 ¹H NMR (400 MHz, DMSO-d₆) δ ppm 8.37 (s, 1H), 7.82-7.71 (m, 1H), 7.66 (s, 1H), 7.23 (s, 1H), 7.06 (s, 2H), 5.02-4.93 (m, 1H), 3.93 (s, 3H), 3.58-3.50 (m, 2H), 1.26 (s, 6H). |
| 61 | 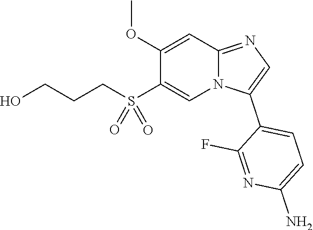 [SMILES: COc1cc2ncc(-c3ccc(N)nc3F)n2cc1S(=O)(=O)CCCO] | 3-((3-(6-amino-2- fluoropyridin-3-yl)-7- methoxyimidazo[1,2- a]pyridin-6- yl)sulfonyl)propan-1- ol | [M + H]⁺ = 381.0 ¹H NMR (400 MHz, DMSO-d₆) δ ppm 8.33 (d, J = 1.2 Hz, 1H), 7.69 (t, J = 8.4, 1H), 7.62 (s, 1H), 7.29 (s, 1H), 6.78 (s, 2H), 6.52-6.47 (m, 1H), 4.64 (t, J = 4.8 Hz, 1H), 3.99 (s, 3H), 3.49- 3.44 (m, 2H), 3.43-3.39 (m, 2H), 1.77-1.66 (m, 2H). |
| 62 | 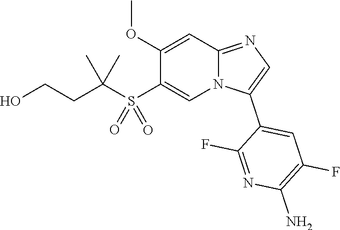 [SMILES: COc1cc2ncc(-c3cc(F)c(N)nc3F)n2cc1S(=O)(=O)C(C)(C)CCO] | 3-((3-(6-amino-2,5- difluoropyridin-3-yl)- 7- methoxyimidazo[1,2- a]pyridin-6- yl)sulfonyl)-3- methylbutan-1-ol | [M + H]⁺ = 426.9 ¹H NMR (400 MHz, DMSO-d₆) δ ppm 8.35 (d, J = 2.4 Hz, 1H), 7.7 (dd, J = 10.4, 7.6 Hz, 1H), 7.66 (s, 1H), 7.27 (s, 1H), 7.06 (s, 2H), 4.53 (t, J = 5.2 Hz, 1H), 3.93 (s, 3H), 3.58-3.51 (m, 2H), 1.82 (t, J = 6.8 Hz, 2H), 1.30 (s, 6H). |
| 63 | 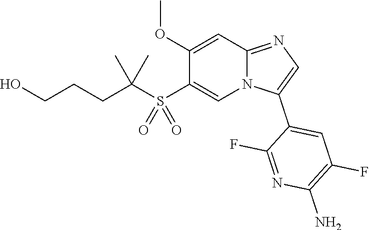 [SMILES: COc1cc2ncc(-c3cc(F)c(N)nc3F)n2cc1S(=O)(=O)C(C)(C)CCCO] | 4-((3-(6-amino-2,5- difluoropyridin-3-yl)- 7- methoxyimidazo[1,2- a]pyridin-6- yl)sulfonyl)-4- methylpentan-1-ol | [M + H]⁺ = 440.8 ¹H NMR (400 MHz, DMSO-d₆) δ ppm 8.35 (d, J = 2.4 Hz, 1H), 7.82-7.72 (m, 1H), 7.67 (s, 1H), 7.26 (s, 1H), 7.06 (s, 2H), 4.44 (t, J = 5.2 Hz, 1H), 3.92 (s, 3H), 3.41-3.32 (m, 2H), 1.71-1.63 (m, 2H), 1.53- 1.41 (m, 2H), 1.25 (s, 6H). |
| 64 | 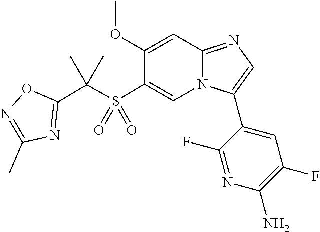 [SMILES: COc1cc2ncc(-c3cc(F)c(N)nc3F)n2cc1S(=O)(=O)C(C)(C)c1nc(C)no1] | 3,6-difluoro-5-(7- methoxy-6-((2-(3- methyl-1,2,4- oxadiazol-5- yl)propan-2- yl)sulfonyl)imidazo[1, 2-a]pyridin-3- yl)pyridin-2-amine | [M + H]⁺ = 465.0 ¹H NMR (400 MHz, DMSO-d₆) δ ppm 8.36 (d, J = 1.6 Hz, 1H), 7.84-7.76 (m, 1H), 7.69 (s, 1H), 7.17 (s, 1H), 7.08 (s, 2H), 3.62 (s, 3H), 2.34 (s, 3H), 1.79 (s, 6H). |
| 65 | 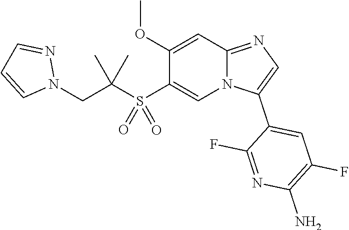 [SMILES: COc1cc2ncc(-c3cc(F)c(N)nc3F)n2cc1S(=O)(=O)C(C)(C)Cn1cccn1] | 3,6-difluoro-5-(7- methoxy-6-((2- methyl-1-(1H-pyrazol- 1-yl)propan-2- yl)sulfonyl)imidazo[1, 2-a]pyridin-3- yl)pyridin-2-amine | [M + H]⁺ = 463.0 ¹H NMR (400 MHz, DMSO-d₆) δ ppm 8.45 (d, J = 2.4 Hz, 1H), 7.84-7.76 (m, 2H), 7.69 (s, 1H), 7.40 (d, J = 1.6 Hz, 1H), 7.30 (s, 1H), 7.07 (s, 2H), 6.22 (t, J = 2.0 Hz, 1H), 4.44 (s, 2H), 3.96 (s, 3H), 1.25 (s, 6H). |
| 66 | 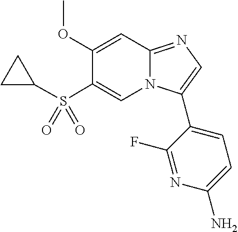 [SMILES: COc1cc2ncc(-c3ccc(N)nc3F)n2cc1S(=O)(=O)C1CC1] | 5-(6- (cyclopropylsulfonyl)- 7- methoxyimidazo[1,2- a]pyridin-3-yl)-6- fluoropyridin-2-amine | [M + H]⁺ = 363.1 ¹H NMR (400 MHz, DMSO-d₆) δ ppm 8.30 (d, J = 2.0 Hz, 1H), 7.73-7.64 (m, 1H), 7.61 (s, 1H), 7.29 (s, 1H), 6.77 (s, 2H), 6.49 (dd, J = 8.4, 2.0 Hz, 1H), 4.01 (s, 3H), 3.10-3.02 (m, 1H), 1.14-1.03 (m, 4H). |
| 67 | 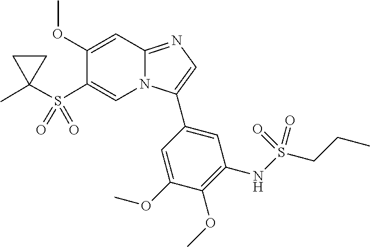 [SMILES: CCCS(=O)(=O)Nc1cc(-c2cnc3cc(OC)c(S(=O)(=O)C4(C)CC4)cn23)cc(OC)c1OC] | N-(2,3-dimethoxy-5- (7-methoxy-6-((1- methylcyclopropyl) sulfonyl)imidazo[1,2- a]pyridin-3- yl)phenyl)propane-1- sulfonamide | [M + H]⁺ = 524.3 ¹H NMR (400 MHz, DMSO-d₆) δ ppm 9.26 (s, 1H), 8.83 (s, 1H), 7.70 (s, 1H), 7.26 (s, 1H), 7.20 (d, J = 2.00 Hz, 1H), 6.99 (br s, 1H), 3.97 (s, 3H), 3.85 (s, 3H), 3.79 (s, 3H), 3.05 (br s, 2H), 1.73 (d, J = 7.60 Hz, 2H), 1.49 (d, J = 2.00 Hz, 2H), 1.30 (s, 3H), 0.97-0.99 (m, 3H), 0.96 (s, 1H), 0.94 (s, 1H) |
| 68 | 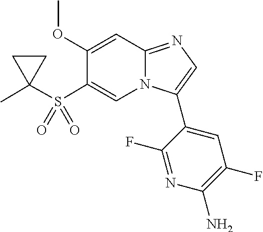 [SMILES: COc1cc2ncc(-c3cc(F)c(N)nc3F)n2cc1S(=O)(=O)C1(C)CC1] | 3,6-difluoro-5-(7- methoxy-6-((1- methylcyclopropyl) sulfonyl)imidazo[1,2- a]pyridin-3-yl)pyridin- 2-amine | [M + H]⁺ = 395.3 ¹H NMR (400 MHz, DMSO-d₆) δ 8.43 (d, J = 2.40 Hz, 1H), 7.81 (dd, J = 10.4, 7.60 Hz, 1H), 7.66 (s, 1H), 7.28 (s, 1H), 7.05 (brs, 2H), 3.97 (s, 3H), 1.48 (q, J = 4.40 Hz, 2H), 1.27 (s, 3H), 0.99 (q, J = 4.40 Hz, 2H). |
| 69 | 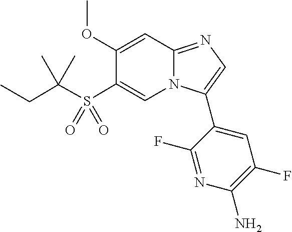 [SMILES: CCC(C)(C)S(=O)(=O)c1cn2c(-c3cc(F)c(N)nc3F)cnc2cc1OC] | 3,6-difluoro-5-(7- methoxy-6-(tert- pentylsulfonyl)imidazo [1,2-a]pyridin-3- yl)pyridin-2-amine | [M + H]⁺ = 411.2 ¹H NMR (400 MHz, DMSO-d₆) δ ppm 8.35 (d, J = 2.0 Hz, 1H), 7.81-7.73 (m, 1H), 7.67 (s, 1H), 7.26 (s, 1H), 7.06 (s, 2H), 3.93 (s, 3H), 1.67 (q, J = 7.6 Hz, 2H), 1.24 (s, 6H), 0.91 (t, J = 7.6 Hz, 3H). |
| 70 | 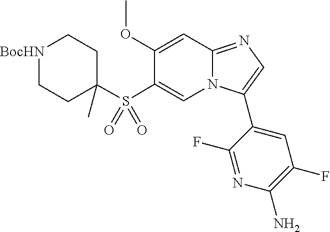 | tert-butyl 4-((3-(6- amino-2,5- difluoropyridin-3-yl)- 7- methoxyimidazo[1,2- a]pyridin-6- yl)sulfonyl)-4- methylpiperidine-1- carboxylate | [M + H]⁺ = 538.1 ¹H NMR (400 MHz, DMSO-d₆) δ ppm 8.37 (d, J = 2.0 Hz, 1H), 7.82-7.73 (m, 1H), 7.68 (s, 1H), 7.27 (s, 1H), 7.06 (s, 2H), 3.93 (s, 3H), 3.89-3.82 (m, 2H), 3.01-2.82 (m, 2H), 1.91- 1.80 (m, 2H), 1.60-1.51 (m, 2H), 1.37 (s, 9H), 1.35 (s, 3H). |
| 71 | 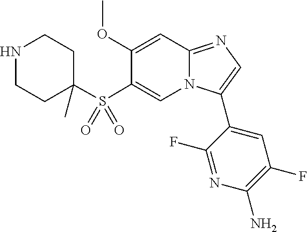 [SMILES: COc1cc2ncc(-c3cc(F)c(N)nc3F)n2cc1S(=O)(=O)C1(C)CCNCC1] | 3,6-difluoro-5-(7- methoxy-6-((4- methylpiperidin-4- yl)sulfonyl)imidazo[1, 2-a]pyridin-3- yl)pyridin-2-amine | [M + H]⁺ = 438.0 ¹H NMR (400 MHz, DMSO-d₆) δ ppm 8.35 (br s, 1H), 8.29 (s, 1H), 7.82-7.72 (m, 1H), 7.68 (s, 1H), 7.28 (s, 1H), 7.07 (s, 2H), 3.93 (s, 3H), 3.05-2.96 (m, 2H), 2.80-2.55 (m, 2H), 2.15- 1.94 (m, 2H), 1.60-1.43 (m, 2H), 1.34 (s, 3H). |
| 72 | 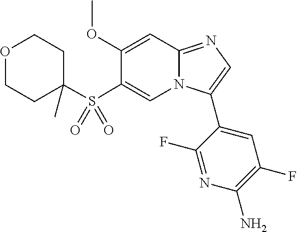 [SMILES: COc1cc2ncc(-c3cc(F)c(N)nc3F)n2cc1S(=O)(=O)C1(C)CCOCC1] | 3,6-difluoro-5-(7- methoxy-6-((4- methyltetrahydro-2H- pyran-4- yl)sulfonyl)imidazo[1, 2-a]pyridin-3- yl)pyridin-2-amine | [M + H]⁺ = 439.1 ¹H NMR (400 MHz, DMSO-d₆) δ ppm 8.38 (d, J = 2.0 Hz, 1H), 7.78 (dd, J = 10.4, 7.6 Hz, 1H), 7.68 (s, 1H), 7.27 (s, 1H), 7.05 (s, 2H), 3.93 (s, 3H), 3.85-3.78 (m, 2H), 3.46 (t, J = 11.6 Hz, 2H), 2.09-2.00 (m, 2H), 1.47 (d, J = 13.2 Hz, 2H), 1.39 (s, 3H). |
| 73 | 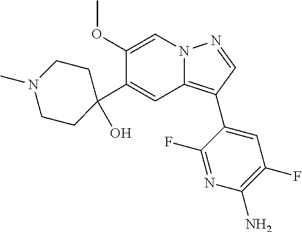 [SMILES: COc1cn2ncc(-c3cc(F)c(N)nc3F)c2cc1C1(O)CCN(C)CC1] | 4-(3-(6-amino-2,5- difluoropyridin-3-yl)- 6- methoxypyrazolo[1,5- a]pyridin-5-yl)-1- methylpiperidin-4-ol | [M + H]⁺ = 390.1 ¹H NMR (400 MHz, DMSO-d₆) δ ppm 8.39 (s, 1H), 8.01 (s, 1H), 7.85 (s, 1H), 7.79-7.70 (m, 1H), 6.66 (s, 2H), 5.17-5.08 (m, 1H), 3.88 (s, 3H), 2.69-2.62 (m, 2H), 2.61-2.52 (m, 4H), 2.26 (s, 3H), 1.41-1.32 (m, 2H). |
| 74 | 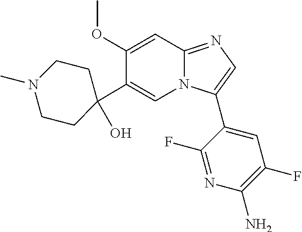 [SMILES: COc1cc2ncc(-c3cc(F)c(N)nc3F)n2cc1C1(O)CCN(C)CC1] | 4-(3-(6-amino-2,5- difluoropyridin-3-yl)- 7- methoxyimidazo[1,2- a]pyridin-6-yl)-1- methylpiperidin-4-ol | [M + H]⁺ = 390.0 ¹H NMR (400 MHz, DMSO-d₆) δ ppm 8.29 (s, 1H), 8.13 (d, J = 1.2 Hz, 1H), 7.78-7.68 (m, 1H), 7.45 (s, 1H), 7.01 (s, 1H), 6.96 (s, 2H), 5.19-4.90 (m, 1H), 3.89 (s, 3H), 2.68-2.50 (m, 4H), 2.46-2.25 (m, 2H), 2.24 (s, 3H), 1.40-1.31 (m, 2H). |
| 75 | 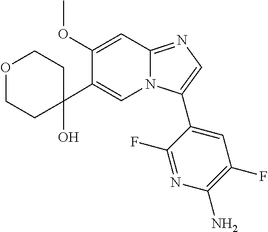 [SMILES: COc1cc2ncc(-c3cc(F)c(N)nc3F)n2cc1C1(O)CCOCC1] | 4-(3-(6-amino-2,5- difluoropyridin-3-yl)- 7- methoxyimidazo[1,2- a]pyridin-6- yl)tetrahydro-2H- pyran-4-ol | [M + H]⁺ = 377.0 ¹H NMR (400 MHz, DMSO-d₆) δ ppm 8.47 (m, 0.1H), 8.13 (s, 1H), 7.73 (dd, J = 10.4, 7.6 Hz, 1H), 7.46 (s, 1H), 7.04 (s, 1H), 6.97 (s, 2H), 5.25 (s, 1H), 3.91 (s, 3H), 3.79-3.62 (m, 4H), 2.59-2.51 (m, 2H), 1.30 (d, J = 13.2 Hz, 2H). |
| 76 | 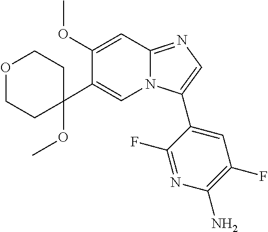 [SMILES: COc1cc2ncc(-c3cc(F)c(N)nc3F)n2cc1C1(OC)CCOCC1] | 3,6-difluoro-5-(7- methoxy-6-(4- methoxytetrahydro- 2H-pyran-4- yl)imidazo[1,2- a]pyridin-3-yl)pyridin- 2-amine | [M + H]⁺ = 391.1 ¹H NMR (400 MHz, DMSO-d₆) δ ppm 7.85- 7.72 (m, 2H), 7.48 (s, 1H), 7.07 (s, 1H), 6.94 (s, 2H), 3.88 (s, 3H), 3.69-3.60 (m, 4H), 3.00 (s, 3H), 2.19-2.09 (m, 2H), 2.08-2.00 (m, 2H). |
| 77 | 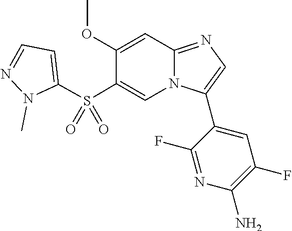 [SMILES: COc1cc2ncc(-c3cc(F)c(N)nc3F)n2cc1S(=O)(=O)c1ccnn1C] | 3,6-difluoro-5-(7- methoxy-6-((1- methyl-1H-pyrazol-5- yl)sulfonyl)imidazo[1, 2-a]pyridin-3- yl)pyridin-2-amine | [M + H]⁺ = 421.0 ¹H NMR (400 MHz, DMSO-d₆) δ ppm 8.72 (d, J = 1.6 Hz, 1H), 7.91-7.82 (m, 1H), 7.68 (s, 1H), 7.64 (d, J = 2.0 Hz, 1H), 7.24 (s, 1H), 7.07 (s, 2H), 7.03 (d, J = 2.0 Hz, 1H), 4.00 (s, 3H), 3.86 (s, 3H). |
| 78 | 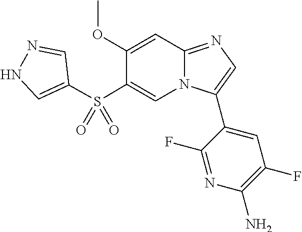 [SMILES: COc1cc2ncc(-c3cc(F)c(N)nc3F)n2cc1S(=O)(=O)c1cn[nH]c1] | 5-(6-((1H-pyrazol-4- yl)sulfonyl)-7- methoxyimidazo[1,2- a]pyridin-3-yl)-3,6- difluoropyridin-2- amine | [M + H]⁺ = 406.9 ¹H NMR (400 MHz, DMSO-d₆) δ ppm 13.99-13.33 (m, 1H), 8.56 (s, 1H), 8.48-8.01 (m, 2H), 7.86 (dd, J = 10.0, 8.0 Hz, 1H), 7.64 (s, 1H), 7.18 (s, 1H), 7.07 (s, 2H), 3.93 (s, 3H). |
| 79 | 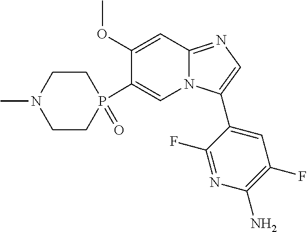 [SMILES: COc1cc2ncc(-c3cc(F)c(N)nc3F)n2cc1P1(=O)CCN(C)CC1] | 4-(3-(6-amino-2,5- difluoropyridin-3-yl)- 7- methoxyimidazo[1,2- a]pyridin-6-yl)-1- methyl-1,4- azaphosphinane 4- oxide | [M + H]⁺ = 408.2 ¹H NMR (400 MHz, DMSO-d₆) δ ppm 8.27- 8.20 (m, 1H), 7.82-7.71 (m, 1H), 7.58 (s, 1H), 7.17 (d, J = 4.4 Hz, 1H), 7.03 (s, 2H), 3.95 (s, 3H), 3.02-2.88 (m, 2H), 2.68-2.62 (m, 2H), 2.44- 2.33 (m, 2H), 2.29 (s, 3H), 1.83-1.73 (m, 2H). |
| 80 | 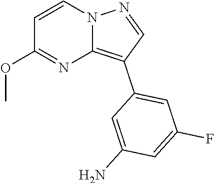 [SMILES: COc1ccn2ncc(-c3cc(N)cc(F)c3)c2n1] | 3-fluoro-5-(5- methoxypyrazolo[1,5- a]pyrimidin-3- yl)aniline | [M + H]⁺ = 259.0 ¹H NMR (400 MHz, DMSO-d₆) δ ppm 8.90 (d, J = 7.6 Hz, 1H), 8.44 (s, 1H), 7.15 (s, 1H), 7.01 (d, J = 10.8 Hz, 1H), 6.60 (d, J = 7.2 Hz, 1H), 6.25-6.12 (m, 1H), 5.40 (s, 2H), 4.05 (s, 3H). |
| 81 | 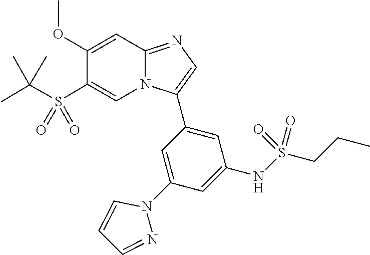 [SMILES: CCCS(=O)(=O)Nc1cc(-c2cnc3cc(OC)c(S(=O)(=O)C(C)(C)C)cn23)cc(-n2cccn2)c1] | N-(3-(6-(tert- butylsulfonyl)-7- methoxyimidazo[1,2- a]pyridin-3-yl)-5-(1H- pyrazol-1- yl)phenyl)propane-1- sulfonamide | [M + H]⁺ = 532.1 ¹H NMR (400 MHz, DMSO-d₆) δ ppm 10.30 (br s, 1H), 8.79 (s, 1H), 8.51 (d, J = 2.4 Hz, 1H), 7.89 (s, 1H), 7.79 (d, J = 1.2 Hz, 3H), 7.35 (s, 1H), 7.31 (s, 1H), 6.63- 6.55 (m, 1H), 3.95 (s, 3H), 3.25-3.17 (m, 2H), 1.80-1.69 (m, 2H), 1.33 (s, 9H), 0.97 (t, J = 7.6 Hz, 3H). |
| 82 | 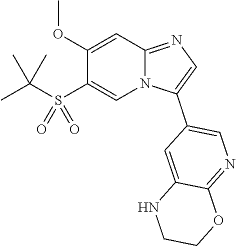 [SMILES: COc1cc2ncc(-c3cnc4c(c3)NCCO4)n2cc1S(=O)(=O)C(C)(C)C] | 7-(6-(tert- butylsulfonyl)-7- methoxyimidazo[1,2- a]pyridin-3-yl)-2,3- dihydro-1H- pyrido[2,3- b][1,4]oxazine | [M + H]⁺ = 402.9 ¹H NMR (400 MHz, DMSO-d₆) δ ppm 8.60 (s, 1H), 8.19 (s, 1H), 7.66 (s, 1H), 7.58-7.55 (m, 1H), 7.25 (s, 1H), 7.06-7.03 (m, 1H), 6.42- 6.39 (m, 1H), 4.34-4.31 (m, 2H), 3.92 (s, 3H), 3.27-3.21 (m, 2H), 1.31 (s, 9H). |
| 83 | 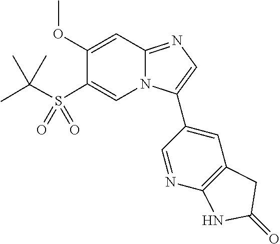 [SMILES: COc1cc2ncc(-c3cnc4c(c3)CC(=O)N4)n2cc1S(=O)(=O)C(C)(C)C] | 5-(6-(tert- butylsulfonyl)-7- methoxyimidazo[1,2- a]pyridin-3-yl)-1,3- dihydro-2H- pyrrolo[2,3-b]pyridin- 2-one | [M + H]⁺ = 401.0 ¹H NMR (400 MHz, DMSO-d₆) δ ppm 11.23 (s, 1H), 8.56 (s, 1H), 8.29 (d, J = 2.0 Hz, 1H), 7.79 (d, J = 1.6 Hz, 1H), 7.71 (s, 1H), 7.27 (s, 1H), 3.93 (s, 3H), 3.66 (s, 2H), 1.30 (s, 9H). |
| 84 | 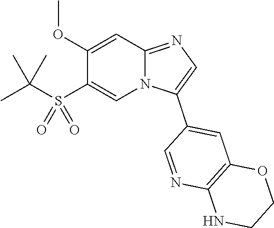 [SMILES: COc1cc2ncc(-c3cnc4c(c3)OCCN4)n2cc1S(=O)(=O)C(C)(C)C] | 7-(6-(tert- butylsulfonyl)-7- methoxyimidazo[1,2- a]pyridin-3-yl)-3,4- dihydro-2H- pyrido[3,2- b][1,4]oxazine | [M + H]⁺ = 403.0 ¹H NMR (400 MHz, DMSO-d₆) δ ppm 8.55 (s, 1H), 7.77 (d, J = 2.0 Hz, 1H), 7.61 (s, 1H), 7.22 (s, 1H), 7.19 (s, 1H), 7.14 (br s, 1H), 4.20-4.14 (m, 2H), 3.92 (s, 3H), 3.49-3.42 (m, 2H), 1.30 (s, 9H). |
| 85 | 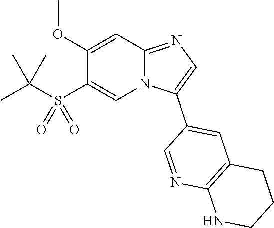 [SMILES: COc1cc2ncc(-c3cnc4c(c3)CCCN4)n2cc1S(=O)(=O)C(C)(C)C] | 6-(6-(tert- butylsulfonyl)-7- methoxyimidazo[1,2- a]pyridin-3-yl)- 1,2,3,4-tetrahydro-1,8- naphthyridine | [M + H]⁺ = 401.1 ¹H NMR (400 MHz, DMSO-d₆) δ ppm 8.52 (s, 1H), 7.95 (s, 1H), 7.57 (s, 1H), 7.34 (s, 1H), 7.21 (s, 1H), 6.84 (br s, 1H), 3.92 (s, 3H), 2.76-2.69 (m, 2H), 2.63- 2.56 (m, 2H), 1.87-1.78 (m, 2H), 1.30 (s, 9H). |
| 86 | 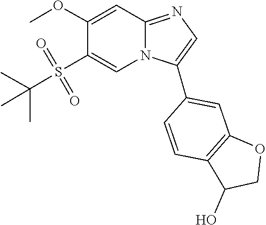 [SMILES: COc1cc2ncc(-c3ccc4c(c3)OCC4O)n2cc1S(=O)(=O)C(C)(C)C] | 6-(6-(tert- butylsulfonyl)-7- methoxyimidazo[1,2- a]pyridin-3-yl)-2,3- dihydrobenzofuran-3- ol | [M + H]⁺ = 403.1 ¹H NMR (400 MHz, DMSO-d₆) δ ppm 8.72 (s, 1H), 7.74 (s, 1H), 7.55 (d, J = 7.6 Hz, 1H), 7.27 (s, 1H), 7.16-7.10 (m, 1H), 7.09 (s, 1H), 5.71 (d, J = 5.6 Hz, 1H), 5.36-5.25 (m, 1H), 4.65- 4.54 (m, 1H), 4.35-4.27 (m, 1H), 3.93 (s, 3H), 1.31 (s, 9H). |
| 87 | 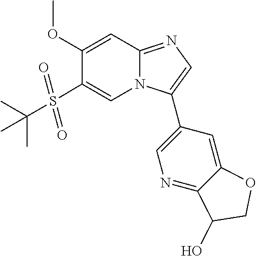 [SMILES: COc1cc2ncc(-c3cnc4c(c3)OCC4O)n2cc1S(=O)(=O)C(C)(C)C] | 6-(6-(tert- butylsulfonyl)-7- methoxyimidazo[1,2- a]pyridin-3-yl)-2,3- dihydrofuro[3,2- b]pyridin-3-ol | [M + H]⁺ = 404.0 ¹H NMR (400 MHz, DMSO-d₆) δ ppm 8.67 (s, 1H), 8.36 (d, J = 1.2 Hz, 1H), 7.85 (s, 1H), 7.57 (d, J = 1.2 Hz, 1H), 7.30 (s, 1H), 5.99 (d, J = 6.0 Hz, 1H), 5.27-5.21 (m, 1H), 4.77-4.70 (m, 1H), 4.46-4.35 (m, 1H), 3.94 (s, 3H), 1.31 (s, 9H). |
| 88 | 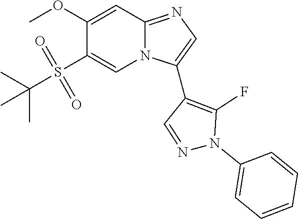 [SMILES: COc1cc2ncc(-c3cnn(-c4ccccc4)c3F)n2cc1S(=O)(=O)C(C)(C)C] | 6-(tert-butylsulfonyl)- 3-(5-fluoro-1-phenyl- 1H-pyrazol-4-yl)-7- methoxyimidazo[1,2- a]pyridine | [M + H]⁺ = 429.1 |
| 89 | 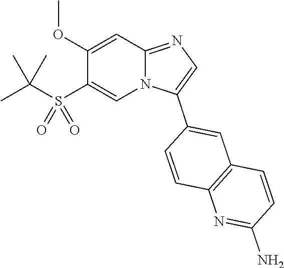 [SMILES: COc1cc2ncc(-c3ccc4nc(N)ccc4c3)n2cc1S(=O)(=O)C(C)(C)C] | 6-(6-(tert- butylsulfonyl)-7- methoxyimidazo[1,2- a]pyridin-3- yl)quinolin-2-amine | [M + H]⁺ = 411.1 ¹H NMR (400 MHz, DMSO-d₆) δ ppm 8.73 (s, 1H), 7.94 (d, J = 8.8 Hz, 1H), 7.84 (d, J = 1.6 Hz, 1H), 7.76 (s, 1H), 7.70-7.66 (m, 1H), 7.63- 7.56 (m, 1H), 7.27 (s, 1H), 6.83 (d, J = 8.8 Hz, 1H), 6.64 (s, 2H), 3.94 (s, 3H), 1.31 (s, 9H). |
| 90 | 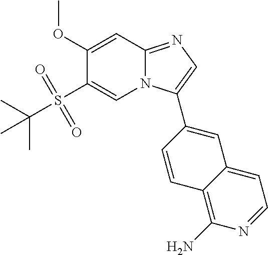 [SMILES: COc1cc2ncc(-c3ccc4c(N)nccc4c3)n2cc1S(=O)(=O)C(C)(C)C] | 6-(6-(tert- butylsulfonyl)-7- methoxyimidazo[1,2- a]pyridin-3- yl)isoquinolin-1-amine | [M + H]⁺ = 411.1 ¹H NMR (400 MHz, DMSO-d₆) δ ppm: 8.83 (s, 1H), 8.39-8.32 (m, 1H), 7.95-7.88 (m, 2H), 7.86 (d, J = 6.0 Hz, 1H), 7.74-7.65 (m, 1H), 7.31 (s, 1H), 6.96-6.90 (m, 1H), 6.89 (br s, 2H), 3.95 (s, 3H), 1.32 (s, 9H). |
| 91 | 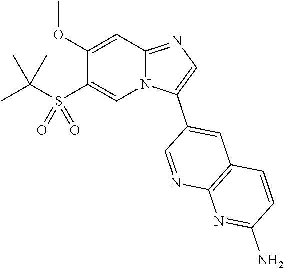 [SMILES: COc1cc2ncc(-c3cnc4nc(N)ccc4c3)n2cc1S(=O)(=O)C(C)(C)C] | 6-(6-(tert- butylsulfonyl)-7- methoxyimidazo[1,2- a]pyridin-3-yl)-1,8- naphthyridin-2-amine | [M + H]⁺ = 412.2 ¹H NMR (400 MHz, DMSO-d₆) δ ppm 8.90 (d, J = 2.4 Hz, 1H), 8.68 (s, 1H), 8.28 (d, J = 2.8 Hz, 1H), 8.01 (d, J = 8.8 Hz, 1H), 7.84 (s, 1H), 7.29 (s, 1H), 7.07 (s, 2H), 6.90 (d, J = 8.8 Hz, 1H), 3.95 (s, 3H), 1.31 (s, 9H). |
| 92 | 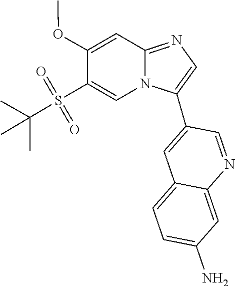 [SMILES: COc1cc2ncc(-c3cnc4cc(N)ccc4c3)n2cc1S(=O)(=O)C(C)(C)C] | 3-(6-(tert- butylsulfonyl)-7- methoxyimidazo[1,2- a]pyridin-3- yl)quinolin-7-amine | [M + H]⁺ = 411.2 |
| 93 | 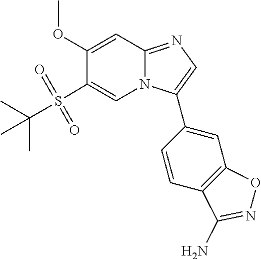 [SMILES: COc1cc2ncc(-c3ccc4c(N)noc4c3)n2cc1S(=O)(=O)C(C)(C)C] | 6-(6-(tert- butylsulfonyl)-7- methoxyimidazo[1,2- a]pyridin-3- yl)benzo[d]isoxazol-3- amine | [M + H]⁺ = 401.0 ¹H NMR (400 MHz, DMSO-d₆) δ ppm: 8.77 (s, 1H), 7.99 (d, J = 8.0 Hz, 1H), 7.87 (s, 1H), 7.73 (s, 1H), 7.50 (dd, J = 1.2, 8.0 Hz, 1H), 7.30 (s, 1H), 6.52 (s, 2H), 3.94 (s, 3H), 1.31 (s, 9H). |
| 94 | 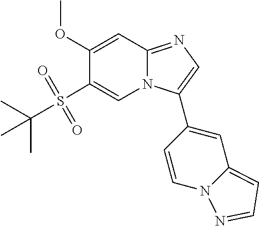 [SMILES: COc1cc2ncc(-c3ccn4nccc4c3)n2cc1S(=O)(=O)C(C)(C)C] | 6-(tert-butylsulfonyl)- 7-methoxy-3- (pyrazolo[1,5- a]pyridin-5- yl)imidazo[1,2- a]pyridine | [M + H]⁺ = 385.1 |
| 95 | 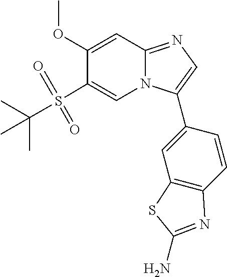 [SMILES: COc1cc2ncc(-c3ccc4nc(N)sc4c3)n2cc1S(=O)(=O)C(C)(C)C] | 6-(6-(tert- butylsulfonyl)-7- methoxyimidazo[1,2- a]pyridin-3- yl)benzo[d]thiazol-2- amine | [M + H]⁺ = 417.1 ¹H NMR (400 MHz, DMSO-d₆) δ ppm: 8.66 (s, 1H), 7.91 (s, 1H), 7.68 (s, 3H), 7.53-7.47 (m, 1H), 7.45-7.38 (m, 1H), 7.25 (s, 1H), 3.93 (s, 3H), 1.30 (s, 9H). |
| 96 | 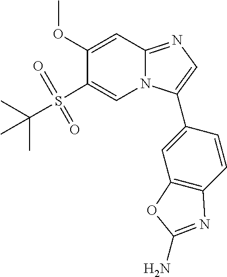 [SMILES: COc1cc2ncc(-c3ccc4nc(N)oc4c3)n2cc1S(=O)(=O)C(C)(C)C] | 6-(6-(tert- butylsulfonyl)-7- methoxyimidazo[1,2- a]pyridin-3- yl)benzo[d]oxazol-2- amine | [M + H]⁺ = 401.0 ¹H NMR (400 MHz, DMSO-d₆) δ ppm 8.67 (s, 1H), 7.69 (s, 1H), 7.60 (s, 3H), 7.42-7.32 (m, 1H), 7.31-7.28 (m, 1H), 7.25 (s, 1H), 3.93 (s, 3H), 1.30 (s, 9H). |
| 97 | 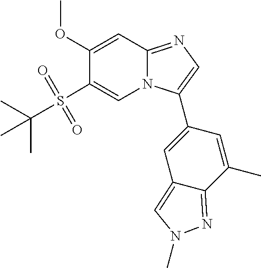 [SMILES: COc1cc2ncc(-c3cc(C)c4nn(C)cc4c3)n2cc1S(=O)(=O)C(C)(C)C] | 5-(6-(tert- butylsulfonyl)-7- methoxyimidazo[1,2- a]pyridin-3-yl)-2,7- dimethyl-2H-indazole | [M + H]⁺ = 413.1 |
| 98 | 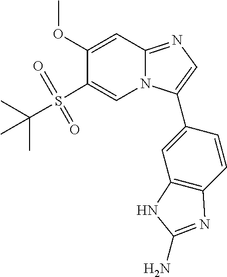 [SMILES: COc1cc2ncc(-c3ccc4nc(N)[nH]c4c3)n2cc1S(=O)(=O)C(C)(C)C] | 6-(6-(tert- butylsulfonyl)-7- methoxyimidazo[1,2- a]pyridin-3-yl)-1H- benzo[d]imidazol-2- amine | [M + H]⁺ = 400.2 ¹H NMR (400 MHz, DMSO-d₆) δ ppm 10.96 (s, 1H), 8.73 (s, 1H), 7.63 (s, 1H), 7.34-7.18 (m, 3H), 7.13-7.00 (m, 1H), 6.35 (s, 2H), 3.93 (s, 3H), 1.31 (s, 9H). |
| 99 | 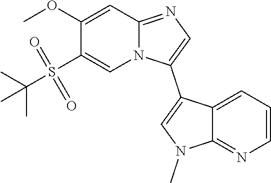 [SMILES: COc1cc2ncc(-c3cn(C)c4ncccc34)n2cc1S(=O)(=O)C(C)(C)C] | 6-(tert-butylsulfonyl)- 7-methoxy-3-(1- methyl-1H- pyrrolo[2,3-b]pyridin- 3-yl)imidazo[1,2- a]pyridine | [M + H]⁺ = 399.1 |
| 100 | 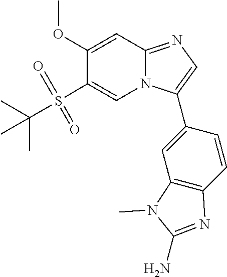 [SMILES: COc1cc2ncc(-c3ccc4nc(N)n(C)c4c3)n2cc1S(=O)(=O)C(C)(C)C] | 6-(6-(tert- butylsulfonyl)-7- methoxyimidazo[1,2- a]pyridin-3-yl)-1- methyl-1H- benzo[d]imidazol-2- amine | [M + H]⁺ = 414.0 ¹H NMR (400 MHz, DMSO-d₆) δ ppm 8.71 (s, 1H), 7.64 (s, 1H), 7.34 (d, J = 1.2 Hz, 1H), 7.29 (d, J = 8.0 Hz, 1H), 7.23 (s, 1H), 7.14 (dd, J = 8.0, 1.6 Hz, 1H), 6.60 (s, 2H), 3.93 (s, 3H), 3.52 (s, 3H), 1.30 (s, 9H). |
| 101 | 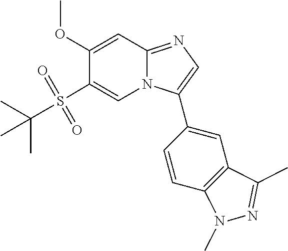 [SMILES: COc1cc2ncc(-c3ccc4c(c3)c(C)nn4C)n2cc1S(=O)(=O)C(C)(C)C] | 5-(6-(tert- butylsulfonyl)-7- methoxyimidazo[1,2- a]pyridin-3-yl)-1,3- dimethyl-1H-indazole | [M + H]⁺ = 413.2 |
| 102 | 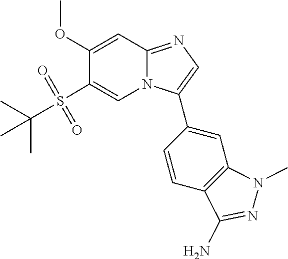 [SMILES: COc1cc2ncc(-c3ccc4c(N)nn(C)c4c3)n2cc1S(=O)(=O)C(C)(C)C] | 6-(6-(tert- butylsulfonyl)-7- methoxyimidazo[1,2- a]pyridin-3-yl)-1- methyl-1H-indazol-3- amine | [M + H]⁺ = 414.2 |
| 103 | 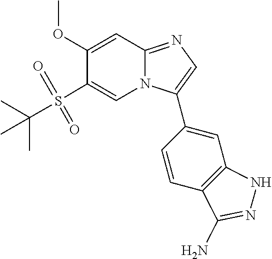 [SMILES: COc1cc2ncc(-c3ccc4c(N)n[nH]c4c3)n2cc1S(=O)(=O)C(C)(C)C] | 6-(6-(tert- butylsulfonyl)-7- methoxyimidazo[1,2- a]pyridin-3-yl)-1H- indazol-3-amine | [M + H]⁺ = 400.0 ¹H NMR (400 MHz, DMSO-d₆) δ ppm 11.64 (s, 1H), 8.77 (s, 1H), 7.86 (d, J = 8.0 Hz, 1H), 7.78 (s, 1H), 7.42 (s, 1H), 7.27 (s, 1H), 7.15- 7.06 (m, 1H), 5.46 (s, 2H), 3.94 (s, 3H), 1.31 (s, 9H). |
| 104 | 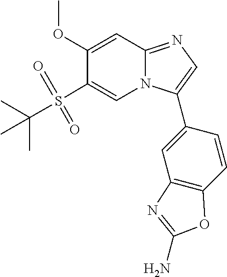 [SMILES: COc1cc2ncc(-c3ccc4oc(N)nc4c3)n2cc1S(=O)(=O)C(C)(C)C] | 5-(6-(tert- butylsulfonyl)-7- methoxyimidazo[1,2- a]pyridin-3- yl)benzo[d]oxazol-2- amine | [M + H]⁺ = 401.2 ¹H NMR (400 MHz, DMSO-d₆) δ ppm 8.69 (s, 1H), 7.70 (s, 1H), 7.59 (s, 2H), 7.53 (d, J = 8.0 Hz, 1H), 7.40 (d, J = 1.6 Hz, 1H), 7.25 (s, 1H), 7.18 (d, J = 1.6 Hz, 1H), 3.93 (s, 3H), 3.90 (s, 9H). |
| 105 | 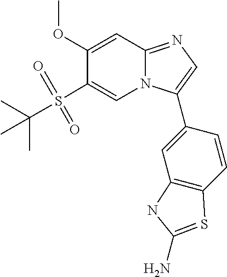 | 5-(6-(tert- butylsulfonyl)-7- methoxyimidazo[1,2- a]pyridin-3- yl)benzo[d]thiazol-2- amine | [M + H]⁺ = 417.2 ¹H NMR (400 MHz, DMSO-d₆) δ ppm 8.75 (s, 1H), 7.88-7.82 (m, 1H), 7.75 (s, 1H), 7.65 (s, 2H), 7.53 (s, 1H), 7.26 (s, 1H), 7.24-7.19 (m, 1H), 3.93 (s, 3H), 1.31 (s, 9H). |
| 106 | 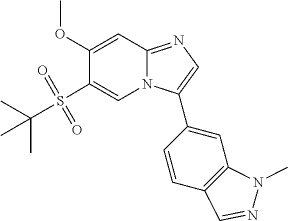 [SMILES: COc1cc2ncc(-c3ccc4cnn(C)c4c3)n2cc1S(=O)(=O)C(C)(C)C] | 6-(6-(tert- butylsulfonyl)-7- methoxyimidazo[1,2- a]pyridin-3-yl)-1- methyl-1H-indazole | [M + H]⁺ = 399.1 |
| 107 | 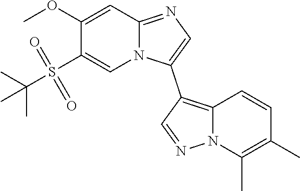 [SMILES: COc1cc2ncc(-c3cnn4c(C)c(C)ccc34)n2cc1S(=O)(=O)C(C)(C)C] | 6-(tert-butylsulfonyl)- 3-(6,7- dimethylpyrazolo[1,5- a]pyridin-3-yl)-7- methoxyimidazo[1,2- a]pyridine | [M + H]⁺ = 413.2 |
| 108 | 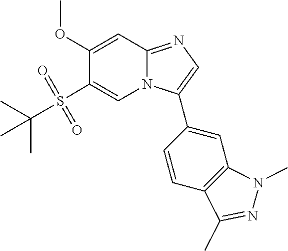 [SMILES: COc1cc2ncc(-c3ccc4c(C)nn(C)c4c3)n2cc1S(=O)(=O)C(C)(C)C] | 6-(6-(tert- butylsulfonyl)-7- methoxyimidazo[1,2- a]pyridin-3-yl)-1,3- dimethyl-1H-indazole | [M + H]⁺ = 413.2 |
| 109 | 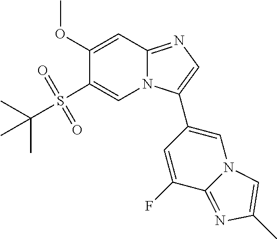 [SMILES: COc1cc2ncc(-c3cc(F)c4nc(C)cn4c3)n2cc1S(=O)(=O)C(C)(C)C] | 6-(tert-butylsulfonyl)- 8′-fluoro-7-methoxy- 2′-methyl-3,6′- biimidazo[1,2- a]pyridine | [M + H]⁺ = 417.1 |
| 110 | 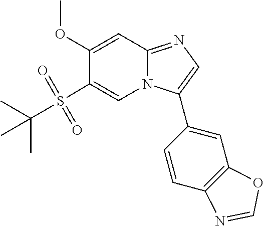 [SMILES: COc1cc2ncc(-c3ccc4ncoc4c3)n2cc1S(=O)(=O)C(C)(C)C] | 6-(6-(tert- butylsulfonyl)-7- methoxyimidazo[1,2- a]pyridin-3- yl)benzo[d]oxazole | [M + H]⁺ = 386.0 |
| 111 | 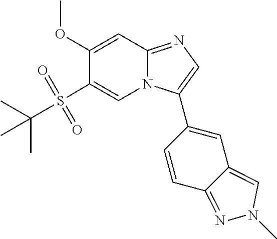 | 5-(6-(tert- butylsulfonyl)-7- methoxyimidazo[1,2- a]pyridin-3-yl)-2- methyl-2H-indazole | [M + H]⁺ = 414.2 |
| 112 | 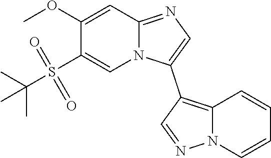 [SMILES: COc1cc2ncc(-c3cnn4ccccc34)n2cc1S(=O)(=O)C(C)(C)C] | 6-(tert-butylsulfonyl)- 7-methoxy-3- (pyrazolo[1,5- a]pyridin-3- yl)imidazo[1,2- a]pyridine | [M + H]⁺ = 385.1 |
| 113 | 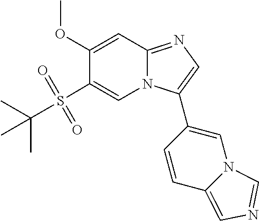 [SMILES: COc1cc2ncc(-c3ccc4cncn4c3)n2cc1S(=O)(=O)C(C)(C)C] | 6-(tert-butylsulfonyl)- 3-(imidazo[1,5- a]pyridin-6-yl)-7- methoxyimidazo[1,2- a]pyridine | [M + H]⁺ = 385.1 |
| 114 | 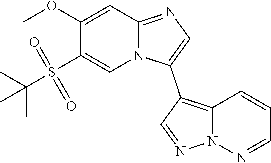 [SMILES: COc1cc2ncc(-c3cnn4ncccc34)n2cc1S(=O)(=O)C(C)(C)C] | 3-(6-(tert- butylsulfonyl)-7- methoxyimidazo[1,2- a]pyridin-3- yl)pyrazolo[1,5- b]pyridazine | [M + H]⁺ = 386.1 |
| 115 | 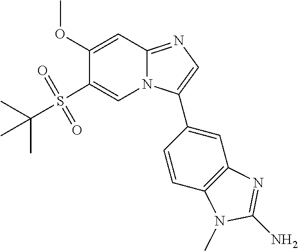 [SMILES: COc1cc2ncc(-c3ccc4c(c3)nc(N)n4C)n2cc1S(=O)(=O)C(C)(C)C] | 5-(6-(tert- butylsulfonyl)-7- methoxyimidazo[1,2- a]pyridin-3-yl)-1- methyl-1H- benzo[d]imidazol-2- amine | [M + H]⁺ = 414.1 ¹H NMR (400 MHz, DMSO-d₆) δ ppm 8.73 (s, 1H), 7.64 (s, 1H), 7.32 (d, J = 8.0 Hz, 1H), 7.30 (d, J = 1.2 Hz, 1H), 7.23 (s, 1H), 7.10-7.07 (m, 1H), 6.57 (s, 2H), 3.93 (s, 3H), 3.56 (s, 3H), 1.30 (s, 9H). |
| 116 | 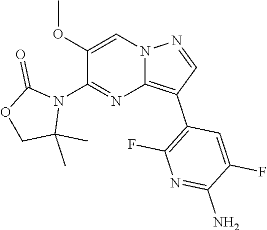 [SMILES: COc1cn2ncc(-c3cc(F)c(N)nc3F)c2nc1N1C(=O)OCC1(C)C] | 3-(3-(6-amino-2,5- difluoropyridin-3-yl)- 6- methoxypyrazolo[1,5- a]pyrimidin-5-yl)-4,4- dimethyloxazolidin-2- one | [M + H]⁺ = 390.9 ¹H NMR (400 MHz, DMSO-d₆) δ ppm 9.09 (s, 1H), 8.34 (d, J = 1.2 Hz, 1H), 8.22 (dd, J = 10.8, 7.6 Hz, 1H), 6.67 (s, 2H), 4.30 (s, 2H), 3.89 (s, 3H), 1.50 (s, 6H). |
| 117 | 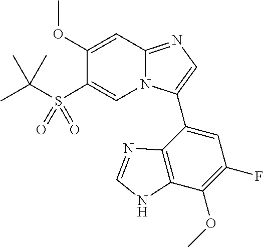 [SMILES: COc1cc2ncc(-c3cc(F)c(OC)c4[nH]cnc34)n2cc1S(=O)(=O)C(C)(C)C] | 4-(6-(tert- butylsulfonyl)-7- methoxyimidazo[1,2- a]pyridin-3-yl)-6- fluoro-7-methoxy-1H- benzo[d]imidazole | [M + H]⁺ = 433.1 ¹H NMR (400 MHz, DMSO-d₆), T = 80° C.) δ ppm 8.90-8.46 (m, 1H), 8.17 (s, 1H), 7.79 (s, 1H), 7.31 (d, J = 12.8 Hz, 1H), 7.24 (s, 1H), 4.21 (s, 3H), 3.97 (s, 3H), 1.34 (s, 9H). |
| 118 | 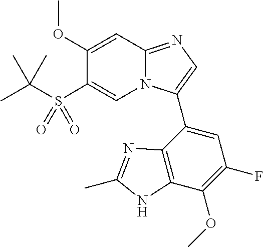 [SMILES: COc1cc2ncc(-c3cc(F)c(OC)c4[nH]c(C)nc34)n2cc1S(=O)(=O)C(C)(C)C] | 4-(6-(tert- butylsulfonyl)-7- methoxyimidazo[1,2- a]pyridin-3-yl)-6- fluoro-7-methoxy-2- methyl-1H- benzo[d]imidazole | [M + H]⁺ = 447.1 ¹H NMR (400 MHz, DMSO-d₆) δ ppm 12.97- 12.16 (m, 1H), 8.80-8.02 (m, 1H), 7.85-7.70 (m, 1H), 7.37-7.22 (m, 2H), 4.35-4.03 (m, 3H), 3.94 (s, 3H), 2.40 (s, 1H), 1.37-1.29 (m, 9H). |
| 119 | 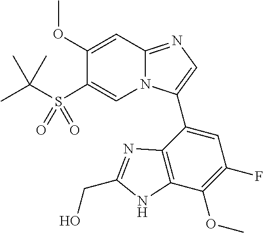 [SMILES: COc1cc2ncc(-c3cc(F)c(OC)c4[nH]c(CO)nc34)n2cc1S(=O)(=O)C(C)(C)C] | (4-(6-(tert- butylsulfonyl)-7- methoxyimidazo[1,2- a]pyridin-3-yl)-6- fluoro-7-methoxy-1H- benzo[d]imidazol-2- yl)methanol | [M + H]⁺ = 463.1 ¹H NMR (400 MHz, DMSO-d₆) δ ppm 13.30- 12.35 (m, 1H), 8.94-7.92 (m, 1H), 7.77 (s, 1H), 7.35-7.22 (m, 2H), 5.53 (s, 1H), 4.55 (d, J = 4.4 Hz, 2H), 4.42-4.03 (m, 3H), 3.94 (s, 3H), 1.33 (s, 9H). |
| 120 | 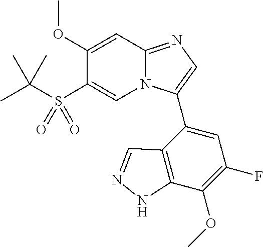 [SMILES: COc1cc2ncc(-c3cc(F)c(OC)c4[nH]ncc34)n2cc1S(=O)(=O)C(C)(C)C] | 4-(6-(tert- butylsulfonyl)-7- methoxyimidazo[1,2- a]pyridin-3-yl)-6- fluoro-7-methoxy-1H- indazole | [M + H]⁺ = 433.0 ¹H NMR (400 MHz, DMSO-d₆) δ ppm 13.75 (br s, 1H), 8.59 (s, 1H), 8.04 (br s, 1H), 7.89 (s, 1H), 7.34-7.30 (m, 2H), 4.14 (s, 3H), 3.95 (s, 3H), 1.31 (s, 9H). |
| 121 | 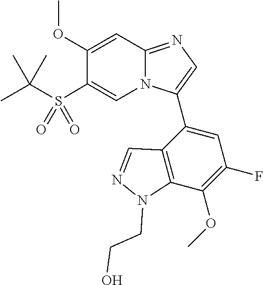 [SMILES: COc1cc2ncc(-c3cc(F)c(OC)c4c3cnn4CCO)n2cc1S(=O)(=O)C(C)(C)C] | 2-(4-(6-(tert- butylsulfonyl)-7- methoxyimidazo[1,2- a]pyridin-3-yl)-6- fluoro-7-methoxy-1H- indazol-1-yl)ethan-1- ol | [M + H]⁺ = 477.1 ¹H NMR (400 MHz, DMSO-d₆) δ ppm 8.61 (s, 1H), 8.03 (s, 1H), 7.89 (s, 1H), 7.41-7.31 (m, 2H), 4.96-4.84 (m, 1H), 4.63 (t, J = 6.0 Hz, 2H), 4.12 (d, J = 1.6 Hz, 3H), 3.95 (s, 3H), 3.87- 3.80 (m, 2H), 1.31 (s, 9H). |
| 122 | 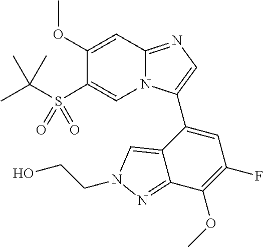 [SMILES: COc1cc2ncc(-c3cc(F)c(OC)c4nn(CCO)cc34)n2cc1S(=O)(=O)C(C)(C)C] | 2-(4-(6-(tert- butylsulfonyl)-7- methoxyimidazo[1,2- a]pyridin-3-yl)-6- fluoro-7-methoxy-2HI- indazol-2-yl)ethan-1- ol | [M + H]⁺ = 477.0 ¹H NMR (400 MHz, DMSO-d₆) δ ppm 8.57 (s, 1H), 8.47 (s, 1H), 7.85 (s, 1H), 7.34-7.24 (m, 2H), 4.98-4.90 (m, 1H), 4.41 (t, J = 5.6 Hz, 2H), 4.26 (s, 3H), 3.95 (s, 3H), 3.87 (s, 2H), 1.31 (s, 9H). |

### Example 123

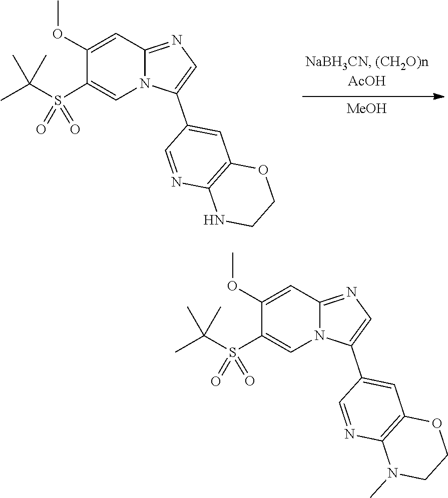

7-(6-(tert-butylsulfonyl)-7-methoxyimidazo[1,2-a]pyridin-3-yl)-4-methyl-3,4-dihydro-2H-pyrido[3,2-b][1,4]oxazine: To a solution of 7-(6-(tert-butylsulfonyl)-7-methoxyimidazo[1,2-a]pyridin-3-yl)-3,4-dihydro-2H-pyrido[3,2-b][1,4]oxazine (25 mg, 62.12 μmol, crude purity, 1 eq) and paraformaldehyde (22.36 mg, 745.40 μmol, 12 eq) in MeOH (2 mL) was added dropwise AcOH (3.73 mg, 62.12 μmol, 3.55 μL, 1 eq) to adjust the pH to 5, and then the mixture was stirred at 25° C. for 12 h under a N₂ atmosphere. To the reaction mixture was added NaBH₃CN (5.86 mg, 93.18 μmol, 1.5 eq) at 0° C., and then the mixture was stirred at 25° C. for 2 h. The reaction mixture was filtered and concentrated under reduced pressure to give a residue, which was purified by RP-HPLC to give the title compound (11.5 mg, 43% yield) as a white solid. [M+H]⁺=417.1. ¹H NMR (400 MHz, DMSO-d₆) δ ppm 8.56 (s, 1H), 7.88 (d, J=2.0 Hz, 1H), 7.63 (s, 1H), 7.23 (s, 1H), 7.18 (d, J=2.0 Hz, 1H), 4.28 (t, J=4.4 Hz, 2H), 3.92 (s, 3H), 3.54-3.47 (m, 2H), 3.10 (s, 3H), 1.30 (s, 9H).

### Example 124

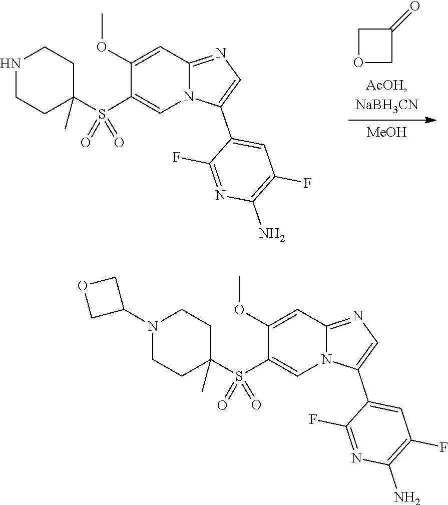 [SMILES: "MeOH" ; COc1cc2ncc(-c3cc(F)c(N)nc3F)n2cc1S(=O)(=O)C1(C)CCNCC1 ; O=C1COC1 ; COc1cc2ncc(-c3cc(F)c(N)nc3F)n2cc1S(=O)(=O)C1(C)CCN(C2COC2)CC1]

3,6-difluoro-5-(7-methoxy-6-((4-methyl-1-(oxetan-3-yl)piperidin-4-yl)sulfonyl)imidazo[1,2-a]pyridin-3-yl)pyridin-2-amine: A mixture of 3,6-difluoro-5-(7-methoxy-6-((4-methylpiperidin-4-yl)sulfonyl)imidazo[1,2-a]pyridin-3-yl)pyridin-2-amine (35 mg, 80.01 μmol, 1 eq), oxetan-3-one (57.65 mg, 800.07 μmol, 10 eq) and AcOH (0.1 mL) in MeOH (1 mL) was stirred at 25° C. for 1 h, and then NaBH₃CN (15.08 mg, 240.02 μmol, 3 eq) was added. The reaction mixture was stirred at 25° C. for 12 h before it was adjusted to pH 7-8 with NH₄OH, filtered and concentrated in vacuo. The resulting crude material was purified via RP-HPLC to yield the title compound as a white solid. LCMS [M+H]⁺=494.1. ¹H NMR (400 MHz, DMSO-d₆) δ ppm 8.37 (d, J=2.0 Hz, 1H), 7.82-7.74 (m, 1H), 7.68 (s, 1H), 7.27 (s, 1H), 7.06 (s, 2H), 4.49 (t, J=6.4 Hz, 2H), 4.38 (t, J=6.0 Hz, 2H), 3.93 (s, 3H), 3.45-3.37 (m, 1H), 2.66-2.59 (m, 2H), 2.09-1.98 (m, 2H), 1.97-1.86 (m, 2H), 1.59-1.51 (m, 2H), 1.29 (s, 3H).

### Example 125, 3,6-difluoro-5-(7-methoxy-6-((1-(2-methoxyethyl)-4-methylpiperidin-4-yl)sulfonyl)imidazo[1,2-a]pyridin-3-yl)pyridin-2-amine, was prepared following procedures analogous to that described for Example 124

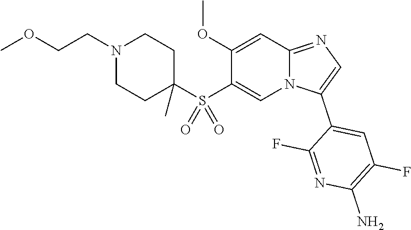 [SMILES: COCCN1CCC(C)(S(=O)(=O)c2cn3c(-c4cc(F)c(N)nc4F)cnc3cc2OC)CC1]

[M+H]⁺=495.9

¹H NMR (400 MHz, DMSO-d₆) δ ppm 8.36 (d, J=2.0 Hz, 1H), 7.82-7.75 (m, 1H), 7.67 (s, 1H), 7.27 (s, 1H), 7.06 (s, 2H), 3.93 (s, 3H), 3.39-3.37 (m, 2H), 3.20 (s, 3H), 2.82-2.78 (m, 2H), 2.47-2.43 (m, 2H), 2.14-2.07 (m, 2H), 2.03-1.94 (m, 2H), 1.54-1.48 (m, 2H), 1.28 (s, 3H).

### Example 126

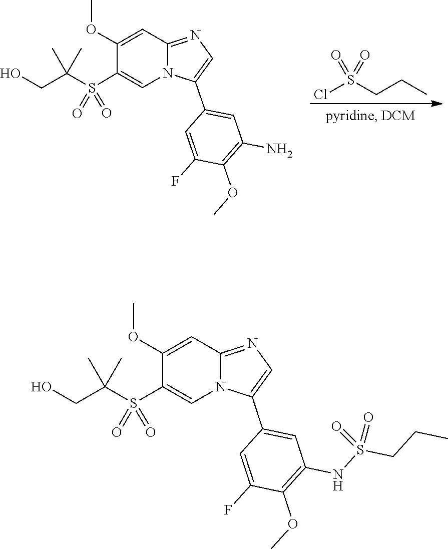

N-(3-fluoro-5-(6-((1-hydroxy-2-methylpropan-2-yl)sulfonyl)-7-methoxyimidazo[1,2-a]pyridin-3-yl)-2-methoxyphenyl)propane-1-sulfonamide: To a solution of 2-((3-(3-amino-5-fluoro-4-methoxyphenyl)-7-methoxyimidazo[1,2-a]pyridin-6-yl)sulfonyl)-2-methylpropan-1-ol (70 mg, 149 μmol, 1 eq) in DCM (5 mL) was added pyridine (235 mg, 2.98 mmol, 240 μL, 20 eq) and propane-1-sulfonyl chloride (424 mg, 2.98 mmol, 335 μL, 20 eq) at 0° C. The mixture was stirred at 20° C. for 2 h before it was diluted with H₂O (30 mL) and extracted with DCM (3×30 mL). The combined organic extracts were dried over Na₂SO₄, filtered, and concentrated under reduced pressure to give a residue, which was purified by column chromatography (SiO₂, 0-10% MeOH/DCM) and RP-HPLC to give the title compound (17.2 mg, 21% yield) as a white solid. [M+H]⁺=530.1. ¹H NMR (400 MHz, DMSO-d₆) δ ppm 9.68 (s, 1H), 8.73 (s, 1H), 8.16 (br s, 1H), 7.50 (s, 1H), 7.47-7.40 (m, 2H), 4.06 (s, 3H), 3.97 (d, J=1.2 Hz, 3H), 3.58 (s, 2H), 3.24-3.16 (m, 2H), 1.83-1.70 (m, 2H), 1.30 (s, 6H), 0.99 (t, J=7.6 Hz, 3H).

The following compounds were prepared following procedures analogous to that described for Example 126. Examples prepared using intermediates or building blocks containing Boc, THP, TBS, dioxolane or other similar protecting groups were subjected to protecting group removal under acidic conditions (generally with HCl or TFA) prior to purification of the final product, as appropriate.

| Ex. | | | |
|---|---|---|---|
| No. | Structure | Name | LCMS, ¹H NMR |
| 127 | 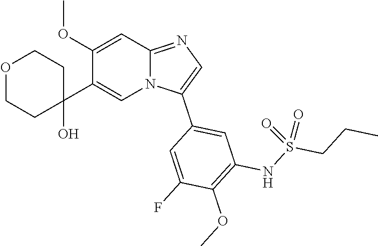 [SMILES: CCCS(=O)(=O)Nc1cc(-c2cnc3cc(OC)c(C4(O)CCOCC4)cn23)cc(F)c1OC] | N-(3-fluoro-5-(6-(4- hydroxytetrahydro- 2H-pyran-4-yl)-7- methoxyimidazo[1,2- a]pyridin-3-yl)-2- methoxyphenyl)propane- 1-sulfonamide | [M + H]⁺ = 494.1 ¹H NMR (400 MHz, DMSO-d₆) δ ppm 9.55 (s, 1H), 8.54 (s, 1H), 7.58 (s, 1H), 7.42-7.31 (m, 2H), 7.06 (s, 1H), 5.23 (s, 1H), 3.93 (s, 3H), 3.92 (s, 3H), 3.82-3.74 (m, 2H), 3.73- 3.67 (m, 2H), 3.20-3.12 (m, 2H), 2.59-2.52 (m, 2H), 1.83-1.72 (m, 2H), 1.39-1.30 (m, 2H), 0.99 (t, J = 7.6 Hz, 3H). |
| 128 | 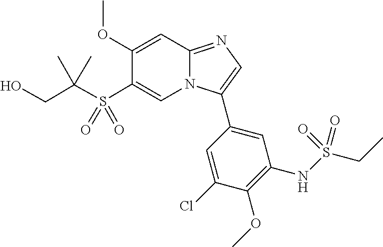 [SMILES: CCS(=O)(=O)Nc1cc(-c2cnc3cc(OC)c(S(=O)(=O)C(C)(C)CO)cn23)cc(Cl)c1OC] | N-(3-chloro-5-(6-((1- hydroxy-2- methylpropan-2- yl)sulfonyl)-7- methoxyimidazo[1,2- a]pyridin-3-yl)-2- methoxyphenyl)ethane sulfonamide | [M + H]⁺ = 532.0 ¹H NMR (400 MHz, DMSO-d₆) δ ppm 9.62 (s, 1H), 8.71 (s, 1H), 7.79 (s, 1H), 7.59 (d, J = 2.4 Hz, 1H), 7.54 (d, J = 2.0 Hz, 1H), 7.24 (s, 1H), 4.96 (t, J = 5.6 Hz, 1H), 3.94 (s, 3H), 3.88 (s, 3H), 3.58 (d, J = 5.6 Hz, 2H), 3.30-3.22 (m, 2H), 1.32-1.24 (m, 9H) |
| 129 | 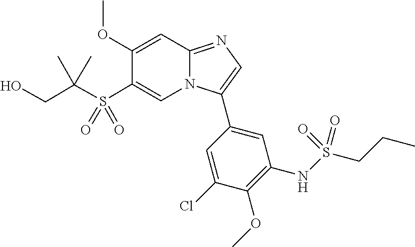 [SMILES: CCCS(=O)(=O)Nc1cc(-c2cnc3cc(OC)c(S(=O)(=O)C(C)(C)CO)cn23)cc(Cl)c1OC] | N-(3-chloro-5-(6-((1- hydroxy-2- methylpropan-2- yl)sulfonyl)-7- methoxyimidazo[1,2- a]pyridin-3-yl)-2- methoxyphenyl) propane-1- sulfonamide | [M + H]⁺ = 545.8 ¹H NMR (400 MHz, DMSO-d₆) δ ppm 8.72 (s, 1H), 7.74 (s, 1H), 7.54 (d, J = 2.0 Hz, 1H), 7.33 (br s, 1H), 7.23 (s, 1H), 4.97 (br s, 1H), 3.93 (s, 3H), 3.85 (s, 3H), 3.58 (s, 2H), 3.12 (t, J = 7.6 Hz, 2H), 1.81- 1.66 (m, 2H), 1.28 (s, 6H), 0.97 (t, J = 7.6 Hz, 3H). |
| 130 | 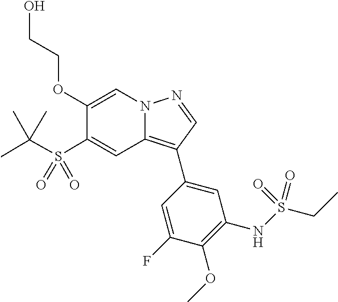 [SMILES: CCS(=O)(=O)Nc1cc(-c2cnn3cc(OCCO)c(S(=O)(=O)C(C)(C)C)cc23)cc(F)c1OC] | N-(5-(5-(tert- butylsulfonyl)-6-(2- hydroxyethoxy)pyrazolo [1,5-a]pyridin-3-yl)- 3-fluoro-2- methoxyphenyl)ethane sulfonamide | [M + H]⁺ = 530.0 ¹H NMR (400 MHz, DMSO-d₆) δ ppm 8.78 (s, 1H), 8.41 (s, 1H), 8.26 (s, 1H), 7.44 (s, 1H), 7.30 (s, 1H), 4.82 (t, J = 5.2 Hz, 1H), 4.16 (t, J = 5.2 Hz, 2H), 3.89 (s, 3H), 3.80- 3.71 (m, 2H), 3.17-3.11 (m, 2H), 1.35 (s, 9H), 1.27 (t, J = 7.2 Hz, 3H). |
| 131 | 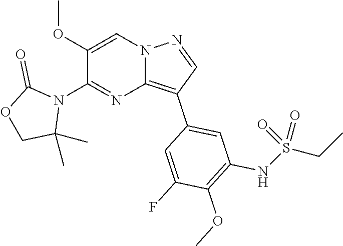 [SMILES: CCS(=O)(=O)Nc1cc(-c2cnn3cc(OC)c(N4C(=O)OCC4(C)C)nc23)cc(F)c1OC] | N-(5-(5-(4,4-dimethyl- 2-oxooxazolidin-3-yl)- 6- methoxypyrazolo[1,5- a]pyrimidin-3-yl)-3- fluoro-2- methoxyphenyl)ethane sulfonamide | [M + H]⁺ = 493.8 ¹H NMR (400 MHz, DMSO-d₆) δ ppm 9.39- 9.29 (m, 1H), 9.11 (s, 1H), 8.63 (s, 1H), 7.86 (s, 1H), 7.74 (dd, J = 3.2, 14.8, 1H), 4.30 (s, 2H), 3.90 (s, 6H), 3.15-3.08 (m, 2H), 1.52 (s, 6H), 1.27 (t, J = 7.2 Hz, 3H). |
| 132 | 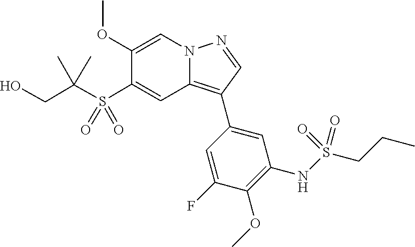 [SMILES: CCCS(=O)(=O)Nc1cc(-c2cnn3cc(OC)c(S(=O)(=O)C(C)(C)CO)cc23)cc(F)c1OC] | N-(3-fluoro-5-(5-((1- hydroxy-2- methylpropan-2- yl)sulfonyl)-6- methoxypyrazolo[1,5- alpyridin-3-yl)-2- methoxyphenyl) propane-1-sulfonamide | [M + H]⁺ = 530.2 ¹H NMR (400 MHz, DMSO-d₆) δ ppm 9.55 (s, 1H), 8.73 (s, 1H), 8.44 (s, 1H), 8.26 (s, 1H), 7.46 (s, 1H), 7.40 (dd, J = 2.0, 12.0 Hz, 1H), 4.99 (t, J = 6.0 Hz, 1H), 3.93 (s, 3H), 3.91 (s, 3H), 3.60 (d, J = 6.0 Hz, 2H), 3.22-3.15 (m, 2H), 1.85-1.72 (m, 2H), 1.30 (s, 6H), 0.99 (t, J = 7.6 Hz, 3H). |
| 133 | 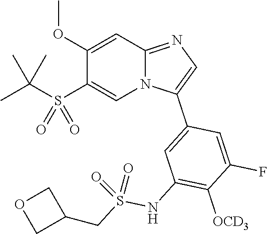 | N-(5-(6-(tert- butylsulfonyl)-7- methoxyimidazo[1,2- a]pyridin-3-yl)-3- fluoro-2-(methoxy- d₃)phenyl)-1-(oxetan- 3- yl)methanesulfonamide | [M + H]⁺ = 545.1 ¹H NMR (400 MHz, DMSO-d₆) δ ppm 9.72 (br s, 1H), 8.71 (s, 1H), 7.78 (s, 1H), 7.45-7.34 (m, 2H), 7.29 (s, 1H), 4.64 (dd, J = 6.4, 8.0 Hz, 2H), 4.43 (t, J = 6.4 Hz, 2H), 3.94 (s, 3H), 3.65 (d, J = 7.6 Hz, 2H), 3.57-3.46 (m, 1H), 1.33 (s, 9H). |
| 134 | 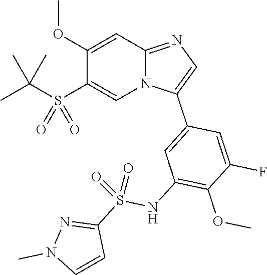 [SMILES: COc1cc2ncc(-c3cc(F)c(OC)c(NS(=O)(=O)c4ccn(C)n4)c3)n2cc1S(=O)(=O)C(C)(C)C] | N-(5-(6-(tert- butylsulfonyl)-7- methoxyimidazo[1,2- a]pyridin-3-yl)-3- fluoro-2- methoxyphenyl)-1- methyl-1H-pyrazole- 3-sulfonamide | [M + H]⁺ = 552.0 ¹H NMR (400 MHz, DMSO-d₆) δ ppm 10.25 (s, 1H), 8.67 (s, 1H), 8.16 (s, 1H), 7.85 (d, J = 1.6 Hz, 1H), 7.56 (s, 1H), 7.50 (s, 1H), 7.42 (dd, J = 11.6, 1.2 Hz, 1H), 6.67 (d, J = 2.0 Hz, 1H), 4.07 (s, 3H), 3.88 (s, 3H), 3.74 (s, 3H), 1.34 (s, 9H). |
| 135 | 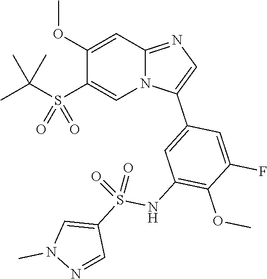 [SMILES: COc1cc2ncc(-c3cc(F)c(OC)c(NS(=O)(=O)c4cnn(C)c4)c3)n2cc1S(=O)(=O)C(C)(C)C] | N-(5-(6-(tert- butylsulfonyl)-7- methoxyimidazo[1,2- a]pyridin-3-yl)-3- fluoro-2- methoxyphenyl)-1- methyl-1H-pyrazole- 4-sulfonamide | [M + H]⁺ = 552.1 ¹H NMR (400 MHz, DMSO-d₆) δ ppm 9.94 (s, 1H), 8.70 (s, 1H), 8.27 (s, 1H), 7.77 (s, 1H), 7.74 (s, 1H), 7.44 (s, 1H), 7.35 (dd, J = 12.0, 1.6 Hz, 1H), 7.29 (s, 1H), 3.94 (s, 3H), 3.83 (s, 3H), 3.73 (d, J = 1.6 Hz, 3H), 1.34 (s, 9H). |
| 136 | 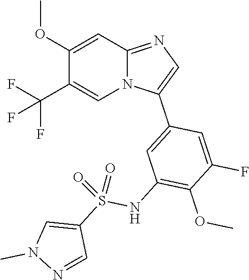 [SMILES: COc1cc2ncc(-c3cc(F)c(OC)c(NS(=O)(=O)c4cnn(C)c4)c3)n2cc1C(F)(F)F] | N-(3-fluoro-2- methoxy-5-(7- methoxy-6- (trifluoromethyl)imidazo [1,2-a]pyridin-3- yl)phenyl)-1-methyl- 1H-pyrazole-4- sulfonamide | [M + H]⁺ = 500.0 |
| 137 | 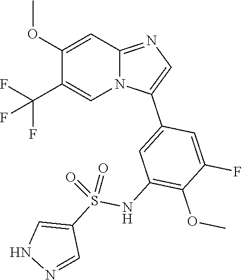 [SMILES: COc1cc2ncc(-c3cc(F)c(OC)c(NS(=O)(=O)c4cn[nH]c4)c3)n2cc1C(F)(F)F] | N-(3-fluoro-2- methoxy-5-(7- methoxy-6- (trifluoromethyl)imidazo [1,2-a]pyridin-3- yl)phenyl)-1H- pyrazole-4- sulfonamide | [M + H]⁺ = 486.0 |
| 138 | 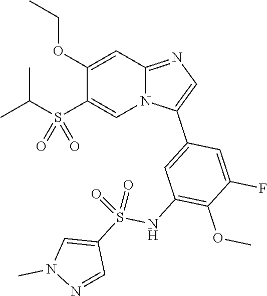 [SMILES: CCOc1cc2ncc(-c3cc(F)c(OC)c(NS(=O)(=O)c4cnn(C)c4)c3)n2cc1S(=O)(=O)C(C)C] | N-(5-(7-ethoxy-6- (isopropylsulfonyl) imidazo [1,2-a]pyridin-3- yl)-3-fluoro-2- methoxyphenyl)-1- methyl-1H-pyrazole- 4-sulfonamide | [M + H]⁺ = 552.0 |
| 139 | 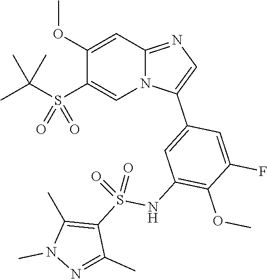 [SMILES: COc1cc2ncc(-c3cc(F)c(OC)c(NS(=O)(=O)c4c(C)nn(C)c4C)c3)n2cc1S(=O)(=O)C(C)(C)C] | N-(5-(6-(tert- butylsulfonyl)-7- methoxyimidazo[1,2- a]pyridin-3-yl)-3- fluoro-2- methoxyphenyl)- 1,3,5-trimethyl-1H- pyrazole-4- sulfonamide | [M + H]⁺ = 580.0 |
| 140 | 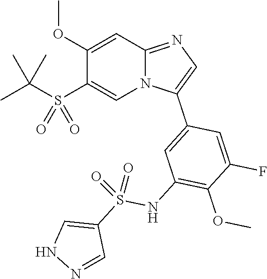 [SMILES: COc1cc2ncc(-c3cc(F)c(OC)c(NS(=O)(=O)c4cn[nH]c4)c3)n2cc1S(=O)(=O)C(C)(C)C] | N-(5-(6-(tert- butylsulfonyl)-7- methoxyimidazo[1,2- a]pyridin-3-yl)-3- fluoro-2- methoxyphenyl)-1H- pyrazole-4- sulfonamide | [M + H]⁺ = 538.1 |
| 141 | 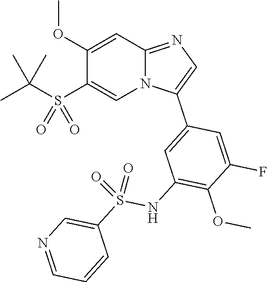 [SMILES: COc1cc2ncc(-c3cc(F)c(OC)c(NS(=O)(=O)c4cccnc4)c3)n2cc1S(=O)(=O)C(C)(C)C] | N-(5-(6-(tert- butylsulfonyl)-7- methoxyimidazo[1,2- a]pyridin-3-yl)-3- fluoro-2- methoxyphenyl) pyridine-3- sulfonamide | [M + H]⁺ = 549.0 ¹H NMR (400 MHz, DMSO-d₆) δ ppm 10.48 (s, 1H), 8.98 (d, J = 2.0 Hz, 1H), 8.79 (dd, J = 4.4, 1.2 Hz, 1H), 8.71 (s, 1H), 8.22- 8.19 (m, 1H), 7.72 (s, 1H), 7.61 (dd, J = 8.0, 4.8 Hz, 1H), 7.42 (s, 1H), 7.35 (d, J = 12.0 Hz, 1H), 7.28 (s, 1H), 3.94 (s, 3H), 3.61 (d, J = 1.6 Hz, 3H), 1.34 (s, 9H). |
| 142 | 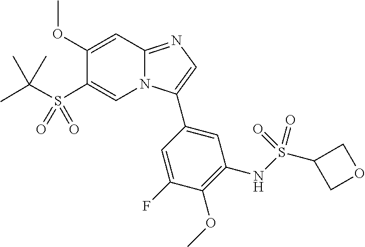 [SMILES: COc1cc2ncc(-c3cc(F)c(OC)c(NS(=O)(=O)C4COC4)c3)n2cc1S(=O)(=O)C(C)(C)C] | N-(5-(6-(tert- butylsulfonyl)-7- methoxyimidazo[1,2- a]pyridin-3-yl)-3- fluoro-2- methoxyphenyl) oxetane-3- sulfonamide | [M + H]⁺ = 528.0 ¹H NMR (400 MHz, DMSO-d₆) δ ppm 9.92 (s, 1H), 8.73 (s, 1H), 7.85- 7.80 (m, 1H), 7.45-7.37 (m, 2H), 7.32-7.22 (m, 1H), 4.83-4.71 (m, 5H), 3.93 (s, 6H), 1.32 (s, 9H). |
| 143 | 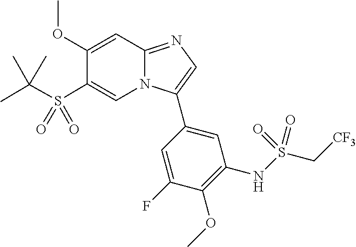 [SMILES: COc1cc2ncc(-c3cc(F)c(OC)c(NS(=O)(=O)CC(F)(F)F)c3)n2cc1S(=O)(=O)C(C)(C)C] | N-(5-(6-(tert- butylsulfonyl)-7- methoxyimidazo[1,2- a]pyridin-3-yl)-3- fluoro-2- methoxyphenyl)- 2,2,2-trifluoroethane- 1-sulfonamide | [M + H]⁺ = 554.0 ¹H NMR (400 MHz, DMSO-d₆) δ ppm 10.28 (s, 1H), 8.69 (s, 1H), 7.78 (s, 1H), 7.46 (d, J = 12.0 Hz, 1H), 7.42 (s, 1H), 7.28 (s, 1H), 4.62-4.54 (m, 2H), 3.95 (s, 3H), 3.93 (s, 3H), 1.32 (s, 9H). |
| 144 | 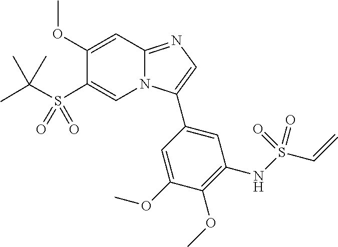 [SMILES: C=CS(=O)(=O)Nc1cc(-c2cnc3cc(OC)c(S(=O)(=O)C(C)(C)C)cn23)cc(OC)c1OC] | N-(5-(6-(tert- butylsulfonyl)-7- methoxyimidazo[1,2- a]pyridin-3-yl)-2,3- dimethoxyphenyl) ethenesulfonamide | [M + H]⁺ = 510.2 ¹H NMR (400 MHz, DMSO-d₆) δ ppm 9.84- 9.11 (m, 1H), 8.73 (s, 1H), 7.73 (s, 1H), 7.26 (s, 1H), 7.16-7.12 (m, 1H), 7.11 (s, 1H), 6.87-6.78 (m, 1H), 6.15 (d, J = 16.8 Hz, 1H), 6.01 (d, J = 10.0 Hz, 1H), 3.93 (s, 3H), 3.86 (s, 3H), 3.80 (s, 3H), 1.33 (s, 9H). |
| 145 | 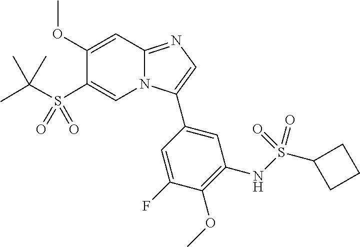 [SMILES: COc1cc2ncc(-c3cc(F)c(OC)c(NS(=O)(=O)C4CCC4)c3)n2cc1S(=O)(=O)C(C)(C)C] | N-(5-(6-(tert- butylsulfonyl)-7- methoxyimidazo[1,2- a]pyridin-3-yl)-3- fluoro-2- methoxyphenyl) cyclobutanesulfonamide | [M + H]⁺ = 526.0 ¹H NMR (400 MHz, DMSO-d₆) δ ppm 8.70 (s, 1H), 7.76 (s, 1H), 7.42 (s, 1H), 7.40-7.33 (m, 1H), 7.28 (s, 1H), 4.09-4.01 (m, 1H), 3.93 (d, J = 4.0 Hz, 6H), 2.39-2.30 (m, 2H), 2.27-2.19 (m, 2H), 1.95- 1.86 (m, 2H), 1.33 (s, 9H). |
| 146 | 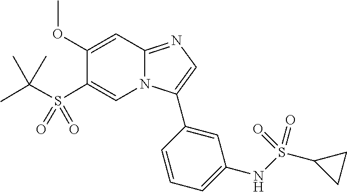 [SMILES: COc1cc2ncc(-c3cccc(NS(=O)(=O)C4CC4)c3)n2cc1S(=O)(=O)C(C)(C)C] | N-(3-(6-(tert- butylsulfonyl)-7- methoxyimidazo[1,2- a]pyridin-3- yl)phenyl) cyclopropane- sulfonamide | [M + H]⁺ = 464.1 ¹H NMR (400 MHz, DMSO-d₆) δ ppm 10.00 (s, 1H), 8.72 (s, 1H), 7.76 (s, 1H), 7.58-7.50 (m, 1H), 7.46 (s, 1H), 7.36-7.26 (m, 3H), 3.94 (s, 3H), 2.72 (quin, J = 6.3 Hz, 1H), 1.32 (s, 9H), 0.97 (d, J = 6.3 Hz, 4H). |
| 147 | 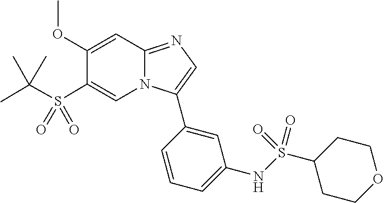 [SMILES: COc1cc2ncc(-c3cccc(NS(=O)(=O)C4CCOCC4)c3)n2cc1S(=O)(=O)C(C)(C)C] | N-(3-(6-(tert- butylsulfonyl)-7- methoxyimidazo[1,2- a]pyridin-3- yl)phenyl)tetrahydro- 2H-pyran-4- sulfonamide | [M + H]⁺ = 507.2 ¹H NMR (400 MHz, DMSO-d₆) δ ppm 8.74 (s, 1H), 7.77 (s, 1H), 7.56- 7.44 (m, 2H), 7.32-7.25 (m, 3H), 3.96-3.88 (m, 5H), 3.28 (s, 3H), 1.91 (d, J = 11.0 Hz, 2H), 1.69 (d, J = 12.2 Hz, 2H), 1.32 (s, 9H). |
| 148 | 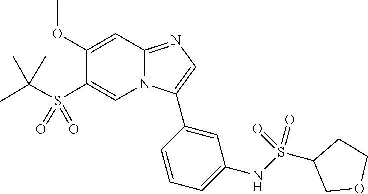 [SMILES: COc1cc2ncc(-c3cccc(NS(=O)(=O)C4CCOC4)c3)n2cc1S(=O)(=O)C(C)(C)C] | N-(3-(6-(tert- butylsulfonyl)-7- methoxyimidazo[1,2- a]pyridin-3- yl)phenyl) tetrahydrofuran- 3-sulfonamide | [M + H]⁺ = 494.1 ¹H NMR (400 MHz, DMSO-d₆) δ ppm 10.23 (s, 1H), 8.74 (s, 1H), 7.78 (s, 1H), 7.59-7.51 (m, 1H), 7.47 (s, 1H), 7.35 (d, J = 8.2 Hz, 1H), 7.29 (s, 2H), 4.05-3.98 (m, 2H), 3.94 (s, 3H), 3.90 (d, J = 9.7 Hz, 1H), 3.84 (d, J = 8.1 Hz, 1H), 3.69-3.61 (m, 1H), 2.19 (q, J = 6.9 Hz, 2H), 1.35-1.30 (m, 9H). |
| 149 | 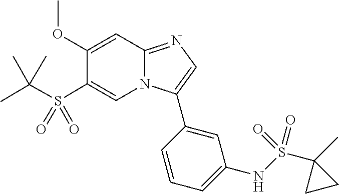 [SMILES: COc1cc2ncc(-c3cccc(NS(=O)(=O)C4(C)CC4)c3)n2cc1S(=O)(=O)C(C)(C)C] | N-(3-(6-(tert- butylsulfonyl)-7- methoxyimidazo[1,2- a]pyridin-3- yl)phenyl)-1- methylcyclopropane- 1-sulfonamide | [M + H]⁺ = 478.1 ¹H NMR (400 MHz, DMSO-d₆) δ ppm 10.00 (s, 1H), 8.69 (s, 1H), 7.74 (s, 1H), 7.56-7.48 (m, 1H), 7.45 (s, 1H), 7.32 (d, J = 6.2 Hz, 2H), 7.28 (s, 1H), 3.93 (s, 3H), 1.43 (s, 3H), 1.31 (s, 9H), 1.24 (d, J = 6.2 Hz, 1H), 1.12 (s, 2H), 0.79 (s, 2H) |
| 150 | 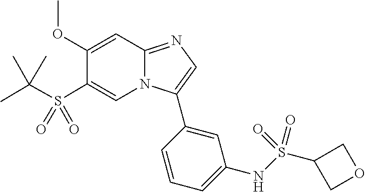 [SMILES: COc1cc2ncc(-c3cccc(NS(=O)(=O)C4COC4)c3)n2cc1S(=O)(=O)C(C)(C)C] | N-(3-(6-(tert- butylsulfonyl)-7- methoxyimidazo[1,2- a]pyridin-3- yl)phenyl)oxetane-3- sulfonamide | [M + H]⁺ = 480.2 ¹H NMR (400 MHz, DMSO-d₆) δ ppm 10.82- 9.75 (m, 1H), 8.71 (s, 1H), 7.77 (s, 1H), 7.52 (s, 1H), 7.42-7.32 (m, 2H), 7.26 (s, 2H), 4.82-4.66 (m, 5H), 3.93 (s, 3H), 1.31 (s, 9H). |
| 151 | 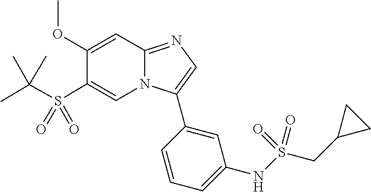 [SMILES: COc1cc2ncc(-c3cccc(NS(=O)(=O)CC4CC4)c3)n2cc1S(=O)(=O)C(C)(C)C] | N-(3-(6-(tert- butylsulfonyl)-7- methoxyimidazo[1,2- a]pyridin-3- yl)phenyl)-1- cyclopropylmethane- sulfonamide | [M + H]⁺ = 478.2 ¹H NMR (400 MHz, DMSO-d₆) δ ppm 10.42- 9.75 (m, 1H), 8.71 (s, 1H), 7.75 (s, 1H), 7.54-7.49 (m, 1H), 7.43 (s, 1H), 7.30- 7.25 (m, 3H), 3.93 (s, 3H), 3.14 (d, J = 7.0 Hz, 2H), 1.31 (s, 9H), 1.06-0.98 (m, 1H), 0.57-0.51 (m, 2H), 0.25 (q, J = 4.8 Hz, 2H). |
| 152 | 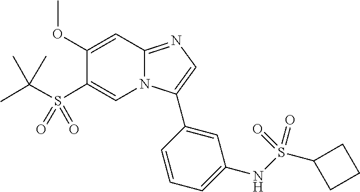 [SMILES: COc1cc2ncc(-c3cccc(NS(=O)(=O)C4CCC4)c3)n2cc1S(=O)(=O)C(C)(C)C] | N-(3-(6-(tert- butylsulfonyl)-7- methoxyimidazo[1,2- a]pyridin-3- yl)phenyl)cyclobutane sulfonamide | [M + H]⁺ = 478.2 ¹H NMR (400 MHz, DMSO-d₆) δ ppm 9.99 (s, 1H), 8.72 (s, 1H), 7.75 (s, 1H), 7.55-7.47 (m, 1H), 7.42 (s, 1H), 7.33-7.22 (m, 3H), 4.02 (quin, J = 8.2 Hz, 1H), 3.94 (s, 3H), 2.33 (dd, J = 2.4, 4.4 Hz, 2H), 2.25-2.15 (m, 2H), 1.96- 1.82 (m, 2H), 1.32 (s, 9H). |
| 153 | 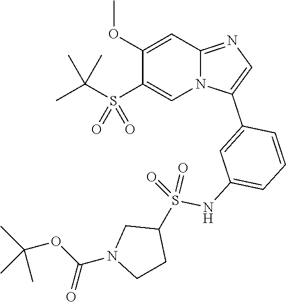 [SMILES: COc1cc2ncc(-c3cccc(NS(=O)(=O)C4CCN(C(=O)OC(C)(C)C)C4)c3)n2cc1S(=O)(=O)C(C)(C)C] | tert-butyl 3-(N-(3-(6- (tert-butylsulfonyl)-7- methoxyimidazo[1,2- a]pyridin-3- yl)phenyl)sulfamoyl) pyrrolidine-1- carboxylate | [M + H]⁺ = 593.3 ¹H NMR (400 MHz, DMSO-d₆) δ ppm 10.33 (s, 1H), 8.75 (s, 1H), 7.77 (s, 1H), 7.57-7.51 (m, 1H), 7.47 (s, 1H), 7.34 (d, J = 7.7 Hz, 1H), 7.31-7.24 (m, 2H), 4.07-3.95 (m, 1H), 3.94 (s, 3H), 3.70- 3.48 (m, 2H), 3.45-3.36 (m, 1H), 3.30 (s, 1H), 2.30- 2.14 (m, 2H), 1.36 (s, 9H), 1.33 (s, 9H). |
| 154 | 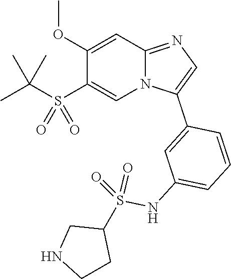 [SMILES: COc1cc2ncc(-c3cccc(NS(=O)(=O)C4CCNC4)c3)n2cc1S(=O)(=O)C(C)(C)C] | N-(3-(6-(tert- butylsulfonyl)-7- methoxyimidazo[1,2- a]pyridin-3- yl)phenyl)pyrrolidine- 3-sulfonamide | [M + H]⁺ = 493.1 ¹H NMR (400 MHz, DMSO-d₆) δ ppm 8.75 (s, 1H), 7.78 (s, 1H), 7.58- 7.51 (m, 1H), 7.47 (s, 1H), 7.34 (d, J = 7.6 Hz, 1H), 7.31-7.26 (m, 2H), 3.94 (s, 3H), 3.80 (s, 1H), 3.15 (s, 2H), 2.94 (s, 1H), 2.87 (s, 1H), 2.10-2.00 (m, 2H), 1.33 (s, 9H). |
| 155 | 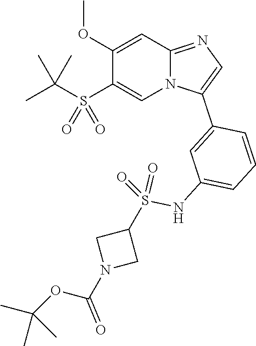 [SMILES: COc1cc2ncc(-c3cccc(NS(=O)(=O)C4CN(C(=O)OC(C)(C)C)C4)c3)n2cc1S(=O)(=O)C(C)(C)C] | tert-butyl 3-(N-(3-(6- (tert-butylsulfonyl)-7- methoxyimidazo[1,2- a]pyridin-3- yl)phenyl)sulfamoyl) azetidine-1-carboxylate | [M + H]⁺ = 579.3 ¹H NMR (400 MHz, DMSO-d₆) δ ppm 10.37 (s, 1H), 8.73 (s, 1H), 7.77 (s, 1H), 7.54 (t, J = 7.8 Hz, 1H), 7.41 (s, 1H), 7.37 (d, J = 7.6 Hz, 1H), 7.32-7.21 (m, 2H), 4.27 (d, J = 4.3 Hz, 1H), 4.12 (s, 2H), 4.05- 3.97 (m, 2H), 3.94 (s, 3H), 1.33 (d, J = 12.7 Hz, 18H). |
| 156 | 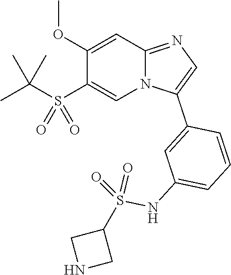 [SMILES: COc1cc2ncc(-c3cccc(NS(=O)(=O)C4CNC4)c3)n2cc1S(=O)(=O)C(C)(C)C] | N-(3-(6-(tert- butylsulfonyl)-7- methoxyimidazo[1,2- a]pyridin-3- yl)phenyl)azetidine-3- sulfonamide | [M + H]⁺ = 479.1 ¹H NMR (500 MHz, DMSO-d₆) δ ppm 8.72 (s, 1H), 8.17 (s, 1H), 7.75 (s, 1H), 7.52-7.45 (m, 1H), 7.40 (s, 1H), 7.31-7.26 (m, 2H), 7.22 (dd, J = 8.0, 1.3 Hz, 1H), 4.40-4.32 (m, 1H), 3.93 (s, 3H), 3.88 (s, 1H), 3.84-3.79 (m, 3H), 3.74 (t, J = 8.9 Hz, 4H), 1.32 (s, 9H). |
| 157 | 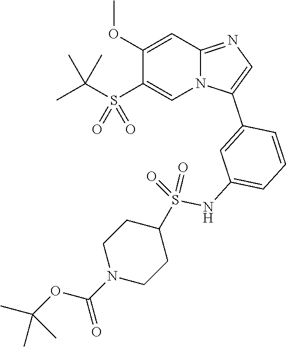 [SMILES: COc1cc2ncc(-c3cccc(NS(=O)(=O)C4CCN(C(=O)OC(C)(C)C)CC4)c3)n2cc1S(=O)(=O)C(C)(C)C] | tert-butyl 4-(N-(3-(6- (tert-butylsulfonyl)-7- methoxyimidazo[1,2- a]pyridin-3- yl)phenyl)sulfamoyl) piperidine-1- carboxylate | [M + H]⁺ = 607.3 ¹H NMR (500 MHz, DMSO-d₆) δ ppm 10.16 (s, 1H), 8.74 (s, 1H), 7.77 (s, 1H), 7.55-7.49 (m, 1H), 7.46 (s, 1H), 7.32-7.26 (m, 3H), 3.97 (s, 2H), 3.93 (s, 3H), 3.35 (s, 1H), 2.75 (s, 2H), 2.00 (d, J = 10.8 Hz, 2H), 1.50 (dq, J = 4.4, 12.5 Hz, 2H), 1.37 (s, 9H), 1.32 (s, 9H). |
| 158 | 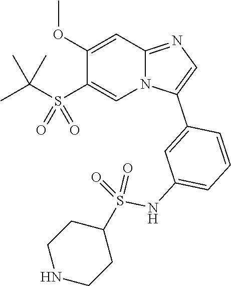 [SMILES: COc1cc2ncc(-c3cccc(NS(=O)(=O)C4CCNCC4)c3)n2cc1S(=O)(=O)C(C)(C)C] | N-(3-(6-(tert- butylsulfonyl)-7- methoxyimidazo[1,2- a]pyridin-3- yl)phenyl)piperidine- 4-sulfonamide | [M + H]⁺ = 507.2 ¹H NMR (500 MHz, DMSO-d₆) δ ppm 8.74 (s, 1H), 8.32 (s, 1H), 7.76 (s, 1H), 7.53-7.48 (m, 1H), 7.46 (s, 1H), 7.31-7.25 (m, 3H), 3.93 (s, 3H), 3.18 (d, J = 19.7 Hz, 3H), 3.03 (d, J = 12.1 Hz, 2H), 1.94 (d, J = 11.9 Hz, 2H), 1.63-1.53 (m, 2H), 1.32 (s, 9H). |
| 159 | 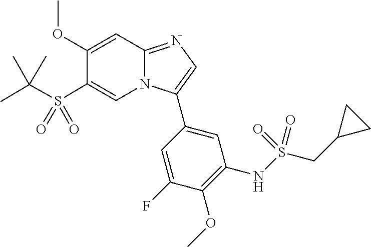 [SMILES: COc1cc2ncc(-c3cc(F)c(OC)c(NS(=O)(=O)CC4CC4)c3)n2cc1S(=O)(=O)C(C)(C)C] | N-(5-(6-(tert- butylsulfonyl)-7- methoxyimidazo[1,2- a]pyridin-3-yl)-3- fluoro-2- methoxyphenyl)-1- cyclopropylmethane- sulfonamide | [M + H]⁺ = 526.1 ¹H NMR (400 MHz, DMSO-d₆) δ ppm 9.53 (s, 1H), 8.69 (s, 1H), 7.75 (s, 1H), 7.44 (s, 1H), 7.36- 7.29 (m, 1H), 7.27 (s, 1H), 3.92 (s, 6H), 3.19-3.16 (m, 2H), 1.32 (s, 9H), 1.10- 1.06 (m, 1H), 0.56-0.50 (m, 2H), 0.34-0.29 (m, 2H) |
| 160 | 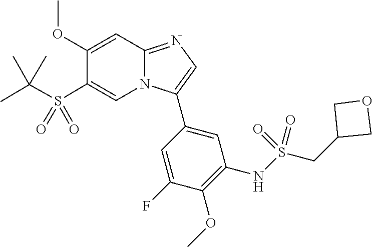 [SMILES: COc1cc2ncc(-c3cc(F)c(OC)c(NS(=O)(=O)CC4COC4)c3)n2cc1S(=O)(=O)C(C)(C)C] | N-(5-(6-(tert- butylsulfonyl)-7- methoxyimidazo[1,2- a]pyridin-3-yl)-3- fluoro-2- methoxyphenyl)-1- (oxetan-3- yl)methanesulfonamide | [M + H]⁺ = 542.2 ¹H NMR (400 MHz, DMSO-d₆) δ ppm 9.75 (br s, 1H), 8.71 (s, 1H), 7.77 (s, 1H), 7.43-7.35 (m, 2H), 7.28 (s, 1H), 4.63 (dd, J = 6.0, 8.0 Hz, 2H), 4.43 (t, J = 6.4 Hz, 2H), 3.94 (s, 3H), 3.93 (s, 3H), 3.65 (d, J = 7.6 Hz, 2H), 3.58-3.46 (m, 1H), 1.33 (s, 9H). |

### Example 161

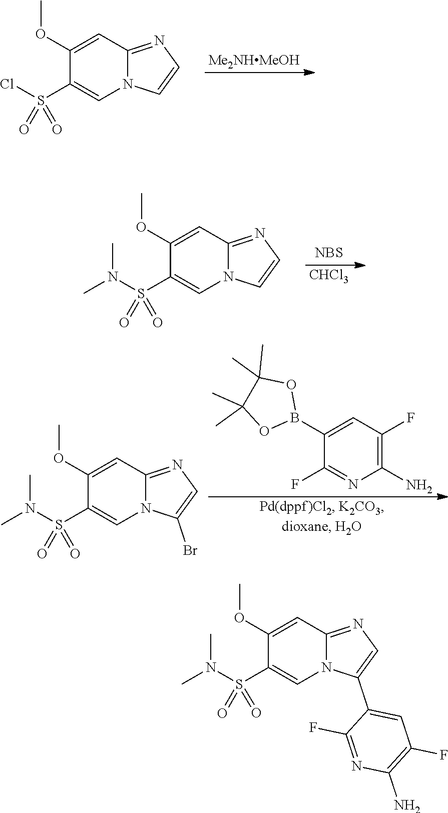

7-methoxy-N,N-dimethylimidazo[1,2-a]pyridine-6-sulfonamide: A mixture of 7-methoxyimidazo[1,2-a]pyridine-6-sulfonyl chloride (400 mg, 1.62 mmol, 1 eq) in Me₂NH in MeOH (25 mL) was degassed and purged with N₂ (3×). The mixture was stirred at 25° C. for 2 hours under a N₂ atmosphere before it was concentrated in vacuo. The resulting crude material was purified by column chromatography (SiO₂, 0-4% MeOH/DCM) to yield the title compound as a white solid. ¹H NMR (400 MHz, DMSO-d₆) δ ppm 9.09 (s, 1H), 7.94 (s, 1H), 7.52 (s, 1H), 7.13 (s, 1H), 3.92 (s, 3H), 2.78 (s, 6H).

3-bromo-7-methoxy-N,N-dimethylimidazo[1,2-a]pyridine-6-sulfonamide: To a solution of 7-methoxy-N,N-dimethylimidazo[1,2-a]pyridine-6-sulfonamide (50 mg, 176.27 μmol, 1 eq) in DMF (1 mL) was added NBS (31.37 mg, 176.27 μmol, 1 eq) at 0° C. The mixture was stirred at 0° C. for 0.5 hour before it was diluted with H₂O (5 mL) and extracted with EtOAc (2×5 mL). The combined organic layers were washed with brine (5 mL), dried over Na₂SO₄, filtered and concentrated under reduced pressure. The resulting crude material was purified by column chromatography (SiO₂, 0-5% MeOH/DCM) to yield the title compound as a white solid. LCMS [M+H]⁺=335.8. ¹H NMR (400 MHz, DMSO-d₆) δ ppm 8.49 (s, 1H), 7.70 (s, 1H), 7.27 (s, 1H), 3.96 (s, 3H), 2.81 (s, 6H).

3-(6-amino-2,5-difluoropyridin-3-yl)-7-methoxy-N,N-dimethylimidazo[1,2-a]pyridine-6-sulfonamide: A mixture of 3-bromo-7-methoxy-N,N-dimethylimidazo[1,2-a]pyridine-6-sulfonamide (40 mg, 107.72 μmol, 1 eq), 3,6-difluoro-5-(4,4,5,5-tetramethyl-1,3,2-dioxaborolan-2-yl)pyridin-2-amine (45.97 mg, 161.59 μmol, 1.5 eq), Pd(dppf)Cl₂ (7.88 mg, 10.77 μmol, 0.1 eq) and K₂CO₃ (44.66 mg, 323.17 μmol, 3 eq) in dioxane (2 mL) and H₂O (0.5 mL) was degassed and purged with N₂ (3×). The reaction mixture was stirred at 80° C. for 2 hours under a N₂ atmosphere before it was concentrated under reduced pressure. The resulting crude material was purified via RP-HPLC to yield the title compound as a white solid. LCMS [M+H]⁺=384.0. ¹H NMR (400 lVUz, DMSO-d₆) δ ppm 8.39 (d, J=2.0 Hz, 1H), 7.86-7.77 (m, 1H), 7.63 (s, 1H), 7.25 (s, 1H), 7.04 (s, 2H), 3.96 (s, 3H), 2.78 (s, 6H).

The following compounds were prepared following procedures analogous to that described for Example 161.

| Ex. | | | |
|---|---|---|---|
| No. | Structure | Name | LCMS, ¹H NMR |
| 162 | 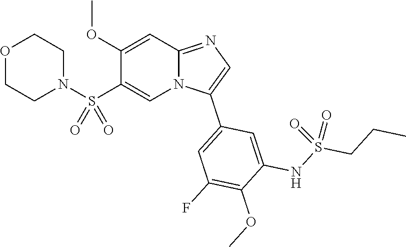 [SMILES: CCCS(=O)(=O)Nc1cc(-c2cnc3cc(OC)c(S(=O)(=O)N4CCOCC4)cn23)cc(F)c1OC] | N-(3-fluoro-2- methoxy-5-(7- methoxy-6- (morpholinosulfonyl) imidazo[1,2- a]pyridin-3- yl)phenyl)propane- 1-sulfonamide | [M + H]⁺ = 543.1 ¹H NMR (400 MHz, DMSO- d₆) δ ppm 9.58 (s, 1H), 8.72 (s, 1H), 7.74 (s, 1H), 7.50-7.38 (m, 2H), 7.26 (s, 1H), 3.97 (s, 3H), 3.93 (s, 3H), 3.63-3.55 (m, 4H), 3.22-3.14 (m, 6H), 1.82-1.68 (m, 2H), 0.98 (t, J = 7.2 Hz, 3H). |
| 163 | 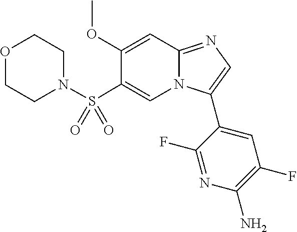 [SMILES: COc1cc2ncc(-c3cc(F)c(N)nc3F)n2cc1S(=O)(=O)N1CCOCC1] | 3,6-difluoro-5-(7- methoxy-6- (morpholinosulfonyl) imidazo[1,2- a]pyridin-3- yl)pyridin-2-amine | [M + H]⁺ = 426.0 ¹H NMR (400 MHz, DMSO- d₆) δ ppm 8.40 (d, J = 2.0 Hz, 1H), 7.88-7.76 (m, 1H), 7.64 (s, 1H), 7.25 (s, 1H), 7.04 (s, 2H), 3.97 (s, 3H), 3.62-3.55 (m, 4H), 3.20-3.12 (m, 4H) |
| 164 | 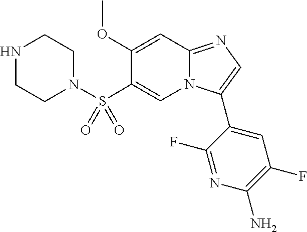 [SMILES: COc1cc2ncc(-c3cc(F)c(N)nc3F)n2cc1S(=O)(=O)N1CCNCC1] | 3,6-difluoro-5-(7- methoxy-6- (piperazin-1- ylsulfonyl)imidazo [1,2-a]pyridin-3- yl)pyridin-2-amine | [M + H]⁺ = 425.0 ¹H NMR (400 MHz, DMSO- d₆) δ ppm 8.38 (d, J = 2.0 Hz, 1H), 7.85-7.76 (m, 1H), 7.64 (s, 1H), 7.24 (s, 1H), 7.04 (s, 2H), 3.96 (s, 3H), 3.25-3.02 (m, 4H), 2.75-2.40 (m, 4H) |
| 165 | 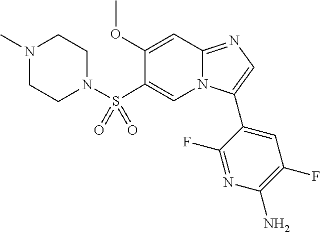 [SMILES: COc1cc2ncc(-c3cc(F)c(N)nc3F)n2cc1S(=O)(=O)N1CCN(C)CC1] | 3,6-difluoro-5-(7- methoxy-6-((4- methylpiperazin-1- yl)sulfonyl)imidazo [1,2-a]pyridin-3- yl)pyridin-2-amine | [M + H]⁺ = 439.0 ¹H NMR (400 MHz, DMSO- d₆) δ ppm 8.39 (s, 1H), 7.2 (dd, J = 10.8, 8.0 1H), 7.63 (s, 1H), 7.24 (s, 1H), 7.04 (s, 2H), 3.96 (s, 3H), 3.21-3.13 (m, 4H), 2.33-2.29 (m, 4H), 2.16 (s, 3H). |

### Example 166

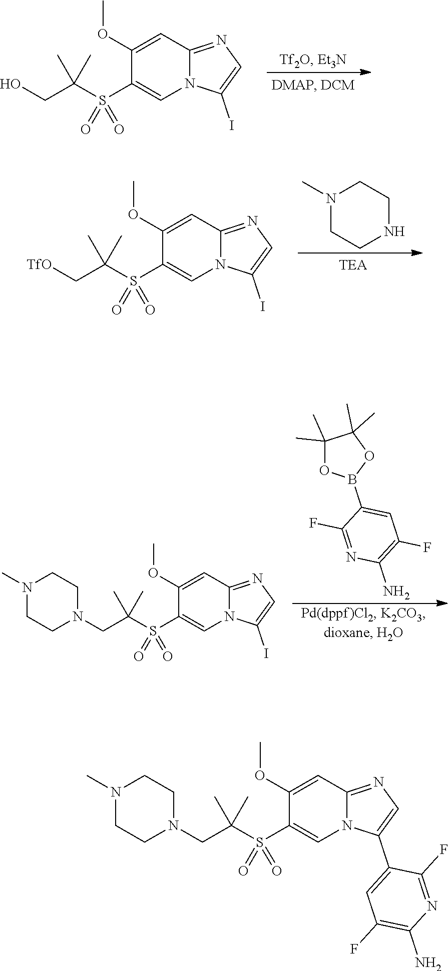

2-((3-iodo-7-methoxyimidazo[1,2-a]pyridin-6-yl)sulfonyl)-2-methylpropyl trifluoromethanesulfonate: To a solution of 2-((3-iodo-7-methoxyimidazo[1,2-a]pyridin-6-yl)sulfonyl)-2-methylpropan-1-ol (110 mg, 241.33 μmol, 1 eq) in DCM (4 mL) was added DMAP (29.48 mg, 241.33 μmol, 1 eq) and Et₃N (134.36 μL, 965.32 μmol, 4 eq). A solution of Tf₂O (79.64 μL, 482.66 μmol, 2 eq) in DCM (2 mL) was added dropwise at 0° C., and then the mixture was stirred at 30° C. for 2 h. After 2 h, the reaction mixture was concentrated in vacuo. The resulting crude material was purified by column chromatography (SiO₂, 0-10% MeOH/DCM) to yield the title compound as a brown solid.

3-iodo-7-methoxy-6-((2-methyl-1-(4-methylpiperazin-1-yl)propan-2-yl)sulfonyl)imidazo[1,2-a]pyridine: To a solution of 2-((3-iodo-7-methoxyimidazo[1,2-a]pyridin-6-yl)sulfonyl)-2-methylpropyl trifluoromethanesulfonate (85 mg, 125.39 μmol, 1 eq) in MeCN (4 mL) was added Et₃N (87.27 μL, 626.97 μmol, 5 eq) and 1-methylpiperazine (41.73 μL, 376.18 μmol, 3 eq). The mixture was stirred at 80° C. for 3 h before it was concentrated in vacuo. The resulting crude material was purified by column chromatography (SiO₂, 0-10% MeOH/DCM) to yield the title compound as a brown solid.

3,6-difluoro-5-(7-methoxy-6-((2-methyl-1-(4-methylpiperazin-1-yl)propan-2-yl)sulfonyl)imidazo[1,2-a]pyridin-3-yl)pyridin-2-amine: A mixture of 3-iodo-7-methoxy-6-((2-methyl-1-(4-methylpiperazin-1-yl)propan-2-yl)sulfonyl)imidazo[1,2-a]pyridine (30 mg, 54.84 μmol, 1 eq), 3,6-difluoro-5-(4,4,5,5-tetramethyl-1,3,2-dioxaborolan-2-yl)pyridin-2-amine (15.60 mg, 54.84 μmol, 1 eq), K₂CO₃ (22.74 mg, 164.51 μmol, 3 eq) and Pd(dppf)Cl₂ (4.01 mg, 5.48 μmol, 0.1 eq) in dioxane (5 mL) and H₂O (1 mL) was degassed and purged with N₂ (3×). The reaction mixture was stirred at 80° C. for 3 h under a N₂ atmosphere before it was concentrated in vacuo. The resulting crude material was purified by RP-HPLC to yield the title compound as a white solid. LCMS [M+H]⁺=495.0. ¹H NMR (400 MHz, DMSO-d₆) δ ppm 8.36 (d, J=2.4 Hz, 1H), 7.82-7.71 (m, 1H), 7.67 (s, 1H), 7.26 (s, 1H), 7.07 (s, 2H), 3.93 (s, 3H), 2.56 (s, 4H), 2.41 (s, 4H), 2.10-2.01 (7, 2H), 2.04 (s, 3H), 1.28 (s, 6H).

The following compounds were prepared following procedures analogous to that described for Example 166.

| Ex. | | | |
|---|---|---|---|
| No. | Structure | Name | LCMS, ¹H NMR |
| 167 | 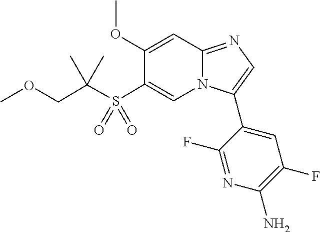 [SMILES: COCC(C)(C)S(=O)(=O)c1cn2c(-c3cc(F)c(N)nc3F)cnc2cc1OC] | 3,6-difluoro-5-(7- methoxy-6-((1- methoxy-2- methylpropan-2- yl)sulfonyl)imidazo [1,2-a]pyridin-3- yl)pyridin-2-amine | [M + H]⁺ = 426.9 ¹H NMR (400 MHz, DMSO-d₆) δ ppm 8.32 (d, J = 2.4 Hz, 1H), 7.77 (dd, J = 10.0, 7.6 Hz, 1H), 7.66 (s, 1H), 7.23 (s, 1H), 7.07 (s, 2H), 3.94 (s, 3H), 3.47 (s, 2H), 3.07 (s, 3H), 1.29 (s, 6H). |
| 168 | 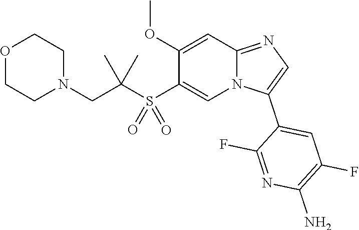 [SMILES: COc1cc2ncc(-c3cc(F)c(N)nc3F)n2cc1S(=O)(=O)C(C)(C)CN1CCOCC1] | 3,6-difluoro-5-(7- methoxy-6-((2- methyl-1- morpholinopropan-2- yl)sulfonyl)imidazo [1,2-a]pyridin-3- yl)pyridin-2-amine | [M + H]⁺ = 482.1 ¹H NMR (400 MHz, DMSO-d₆) δ ppm 8.36 (d, J = 2.0 Hz, 1H), 7.82-7.73 (m, 1H), 7.67 (s, 1H), 7.27 (s, 1H), 7.07 (s, 2H), 3.94 (s, 3H), 3.38 (s, 4H), 2.58 (s, 2H), 2.44-2.37 (m, 4H), 1.30 (s, 6H) |

### Example 169

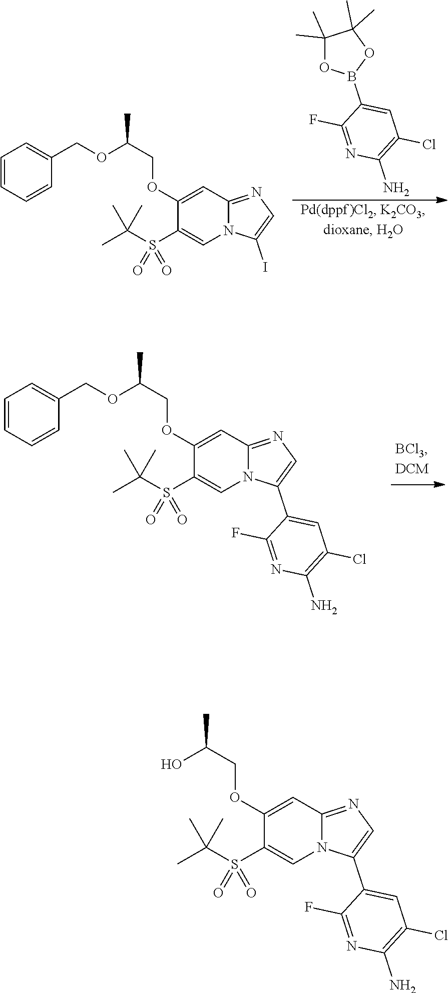

(S)-5-(7-(2-(benzyloxy)propoxy)-6-(tert-butylsulfonyl)imidazo[1,2-a]pyridin-3-yl)-3-chloro-6-fluoropyridin-2-amine: A mixture of (S)-7-(2-(benzyloxy)propoxy)-6-(tert-butylsulfonyl)-3-iodoimidazo[1,2-a]pyridine (25, mg, 42.58 μmol, 1 eq), 3-chloro-6-fluoro-5-(4,4,5,5-tetramethyl-1,3,2-dioxaborolan-2-yl)pyridin-2-amine (19.34 mg, 63.87 μmol, 1.5 eq), Pd(dppf)Cl₂ (3.12 mg, 4.26 μmol, 0.1 eq), and K₂CO₃ (17.65 mg, 127.74 μmol, 3 eq) in dioxane (0.5 mL) and H₂O (0.1 mL) was degassed and purged with N₂ 3 times, and then the mixture was stirred at 100° C. for 1 hour under a N₂ atmosphere. The reaction mixture was diluted with H₂O (20 mL) and extracted with EtOAc (3×10 mL). The combined organic layers were washed with brine (10 mL), dried over Na₂SO₄, filtered and concentrated under reduced pressure to give a residue, which was purified by column chromatography (SiO₂, petroleum ether/EtOAc=10/1 to 0/1) to give the title compound (12 mg, 46% yield) as a yellow solid. ¹H NMR (400 MHz, DMSO-d₆) δ ppm 8.37 (s, 1H), 7.97-7.91 (m, 1H), 7.69 (s, 1H), 7.59-7.55 (m, 1H), 7.38-7.23 (m, 5H), 7.14 (s, 2H), 4.62 (s, 1H), 4.24-4.15 (m, 2H), 3.94 (s, 2H), 1.31-1.28 (m, 12H).

(S)-1-((3-(6-amino-5-chloro-2-fluoropyridin-3-yl)-6-(tert-butylsulfonyl)imidazo[1,2-a]pyridin-7-yl)oxy)propan-2-ol: To a solution of (S)-5-(7-(2-(benzyloxy)propoxy)-6-(tert-butylsulfonyl)imidazo[1,2-a]pyridin-3-yl)-3-chloro-6-fluoropyridin-2-amine (12 mg, 19.74 μmol, 1 eq) in DCM (0.5 mL) was added BCl₃ (6.94 mg, 59.23 μmol, 7.70 μL, 3 eq) at 0° C. The mixture was stirred at 25° C. for 1 hour before it was diluted with H₂O (20 mL) and extracted with EtOAc (3×10 mL). The combined organic layers were washed with brine (10 mL), dried over Na₂SO₄, filtered and concentrated under reduced pressure to give a residue, which was purified by RP-HPLC to give the title compound (4.7 mg, 49% yield) as a yellow solid. [M+H]⁺=457.0. ¹H NMR (400 MHz, DMSO-d₆) δ ppm 8.36 (d, J=2.0 Hz, 1H), 7.94 (d, J=8.4 Hz, 1H), 7.68 (s, 1H), 7.25 (s, 1H), 7.13 (s, 2H), 4.89-4.75 (m, 1H), 4.06-3.92 (m, 3H), 1.31 (s, 9H), 1.22 (d, J=5.6 Hz, 3H). SFC: Rₜ=1.405 min, 100% ee; CHIRALPAK AD-3 50×4.6 mm, 3 μm; 5-40% MeOH (0.05% DEA) in CO₂, 3 mL/min; 35° C., 100 Bar, 220 nm.

The following compounds were prepared following procedures analogous to that described for Example 169. Examples 175 and 176 were obtained from racemic Example 174 by chiral SFC separation.

| Ex. | | | |
|---|---|---|---|
| No. | Structure | Name | LCMS, ¹H NMR |
| 170 | 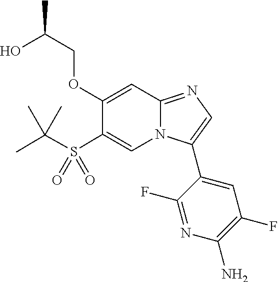 [SMILES: C[C@H](O)COc1cc2ncc(-c3cc(F)c(N)nc3F)n2cc1S(=O)(=O)C(C)(C)C] | (S)-1-((3-(6-amino- 2,5-difluoropyridin-3- yl)-6-(tert- butylsulfonyl)imidazo [1,2-a]pyridin-7- yl)oxy)propan-2-ol | [M + H]⁺ = 441.0 ¹H NMR (400 MHz, DMSO- d₆) δ ppm 8.36 (d, J = 2.4 Hz, 1H), 7.76 (dd, J = 10.0, 7.2 Hz, 1H), 7.66 (s, 1H), 7.25 (s, 1H), 7.05 (s, 2H), 4.78 (d, J = 4.0 Hz, 1H), 4.06-3.94 (m, 3H), 1.32 (s, 9H), 1.21 (d, J = 6.0 Hz, 3H). SFC: Rₜ = 1.109 min, 98% ee; CHIRALPAK AS-3 50 × 4.6 mm, 3 μm; 5-40% MeOH (0.05% DEA) in CO₂, 3 mL/min; 35° C., 100 Bar, 220 nm |
| 171 | 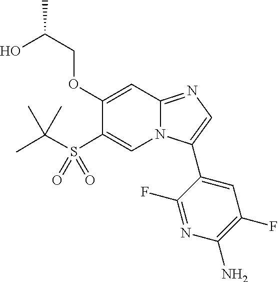 [SMILES: C[C@@H](O)COc1cc2ncc(-c3cc(F)c(N)nc3F)n2cc1S(=O)(=O)C(C)(C)C] | (R)-1-((3-(6-amino- 2,5-difluoropyridin-3- yl)-6-(tert- butylsulfonyl)imidazo [1,2-a]pyridin-7- yl)oxy)propan-2-ol | [M + H]⁺ = 440.8 ¹H NMR (400 MHz, DMSO- d₆) δ ppm 8.36 (d, J = 2.0 Hz, 1H), 7.80-7.71 (m, 1H), 7.67 (s, 1H), 7.25 (s, 1H), 7.06 (s, 2H), 4.79 (d, J = 3.6 Hz, 1H), 4.08-3.92 (m, 3H), 1.32 (s, 9H), 1.22 (d, J = 5.6 Hz, 3H). SFC: Rₜ = 0.959 min, 96% ee; CHIRALPAK IC-3 50 × 4.6 mm, 3 μm; 40% MeOH/MeCN (3/1, 0.05% DEA) in CO₂, 3 mL/min; 35° C., 100 Bar, 220 nm |
| 172 | 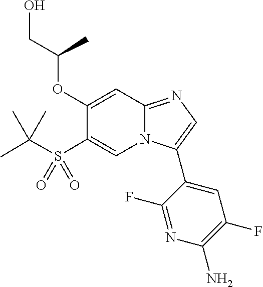 [SMILES: C[C@H](CO)Oc1cc2ncc(-c3cc(F)c(N)nc3F)n2cc1S(=O)(=O)C(C)(C)C] | (R)-2-((3-(6-amino- 2,5-difluoropyridin-3- yl)-6-(tert- butylsulfonyl)imidazo [1,2-a]pyridin-7- yl)oxy)propan-1-ol | [M + H]⁺ = 441.1 ¹H NMR (400 MHz, DMSO- d₆) δ ppm 8.37 (d, J = 2.8 Hz, 1H), 7.76 (dd, J = 10.0, 7.6 Hz, 1H), 7.66 (s, 1H), 7.33 (s, 1H), 7.06 (s, 2H), 4.80-4.76 (m, 1H), 4.75-4.69 (m, 1H), 3.65-3.69 (m, 1H), 3.57-3.50 (m, 1H), 1.32 (s, 9H), 1.29 (d, J = 6.4 Hz, 3H). SFC: Rₜ = 1.160 min, 100% ee; CHIRALPAK AD-3 50 × 4.6 mm, 3 μm; 5-40% MeOH (0.05% DEA) in CO₂, 3 mL/min; 35° C., 100 Bar, 220 nm |
| 173 | 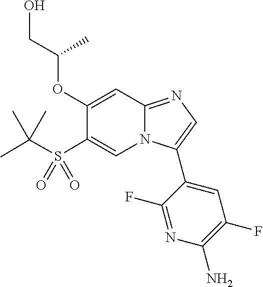 [SMILES: C[C@@H](CO)Oc1cc2ncc(-c3cc(F)c(N)nc3F)n2cc1S(=O)(=O)C(C)(C)C] | (S)-2-((3-(6-amino- 2,5-difluoropyridin-3- yl)-6-(tert- butylsulfonyl)imidazo [1,2-a]pyridin-7- yl)oxy)propan-1-ol | [M + H]⁺ = 441.0 ¹H NMR (400 MHz, DMSO- d₆) δ ppm 8.50-8.44 (m, 1H), 8.37 (d, J = 2.4 Hz, 1H), 7.76 (dd, J = 10.4, 7.2 Hz, 1H), 7.66 (s, 1H), 7.33 (s, 1H), 7.06 (s, 2H), 4.83-4.76 (m, 1H), 4.75-4.68 (m, 1H), 3.65-3.50 (m, 2H), 1.34-1.27 (m, 12H). SFC: Rₜ = 1.239 min, 100% ee; CHIRALPAK AD-3 50 × 4.6 mm, 3 μm; 5-40% MeOH (0.05% DEA) in CO₂, 3 mL/min; 35° C., 100 Bar, 220 nm |
| 174 | 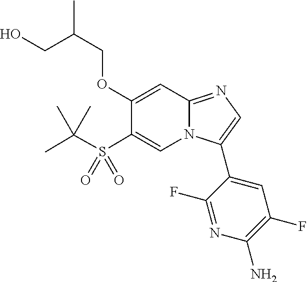 [SMILES: CC(CO)COc1cc2ncc(-c3cc(F)c(N)nc3F)n2cc1S(=O)(=O)C(C)(C)C] | 3-((3-(6-amino-2,5- difluoropyridin-3-yl)- 6-(tert- butylsulfonyl)imidazo [1,2-a]pyridin-7- yl)oxy)-2- methylpropan-1-ol | [M + H]⁺ = 455.2 ¹H NMR (400 MHz, DMSO- d₆) δ ppm 8.35 (d, J = 2.0 Hz, 1H), 7.81-7.73 (m, 1H), 7.66 (s, 1H), 7.21 (s, 1H), 7.07 (s, 2H), 4.59 (br s, 1H), 4.12-3.97 (m, 2H), 3.51-3.40 (m, 2H), 2.10-1.97 (m, 1H), 1.31 (s, 6H), 1.00 (t, J = 7.6 Hz, 3H). |
| 175 | 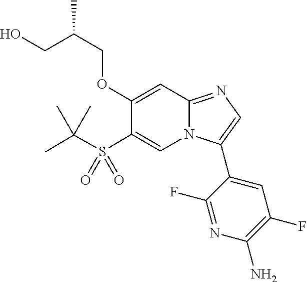 [SMILES: C[C@@H](CO)COc1cc2ncc(-c3cc(F)c(N)nc3F)n2cc1S(=O)(=O)C(C)(C)C] | (S)-3-((3-(6-amino- 2,5-difluoropyridin-3- yl)-6-(tert- butylsulfonyl)imidazo [1,2-a]pyridin-7- yl)oxy)-2- methylpropan-1-ol | [M + H]⁺ = 455.1 ¹H NMR (400 MHz, DMSO- d₆) δ ppm 8.36 (d, J = 2.0 Hz, 1H), 7.77 (dd, J = 10.0, 7.2 Hz, 1H), 7.66 (s, 1H), 7.21 (s, 1H), 7.06 (s, 2H), 4.57 (t, J = 5.2 Hz, 1H), 4.12-4.07 (m, 1H), 4.04-3.98 (m, 1H), 3.50-3.40 (m, 2H), 2.07-2.00 (m, 1H), 1.31 (s, 9H), 1.01 (d, J = 6.8 Hz, 3H). SFC: Rₜ = 0.927 min, 100% ee; CHIRALPAK IC-3 50 × 4.6 mm, 3 μm; 40% MeOH/MeCN (3/1, 0.05% DEA) in CO₂, 3 mL/min; 35° C., 100 Bar, 220 nm |
| 176 | 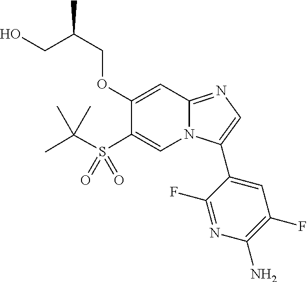 [SMILES: C[C@H](CO)COc1cc2ncc(-c3cc(F)c(N)nc3F)n2cc1S(=O)(=O)C(C)(C)C] | (R)-3-((3-(6-amino- 2,5-difluoropyridin-3- yl)-6-(tert- butylsulfonyl)imidazo [1,2-a]pyridin-7- yl)oxy)-2- methylpropan-1-ol | [M + H]⁺ = 455.1 ¹H NMR (400 MHz, DMSO- d₆) δ ppm 8.36 (d, J = 2.4 Hz, 1H), 7.77 (dd, J = 10.4, 7.6 Hz, 1H), 7.66 (s, 1H), 7.21 (s, 1H), 7.05 (s, 2H), 4.57 (t, J = 5.6 Hz, 1H), 4.12-4.04 (m, 1H), 4.04-3.98 (m, 1H), 3.51-3.41 (m, 2H), 2.06-1.99 (m, 1H), 1.31 (s, 9H), 1.01 (d, J = 7.2 Hz, 3H). SFC: Rₜ = 1.347 min, 99% ee; CHIRALPAK IC-3 50 × 4.6 mm, 3 μm; 40% MeOH/MeCN (3/1, 0.05% DEA) in CO₂, 3 mL/min; 35° C., 100 Bar, 220 nm |
| 177 | 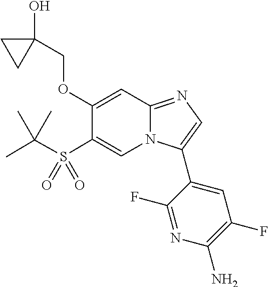 [SMILES: CC(C)(C)S(=O)(=O)c1cn2c(-c3cc(F)c(N)nc3F)cnc2cc1OCC1(O)CC1] | 1-(((3-(6-amino-2,5- difluoropyridin-3-yl)- 6-(tert- butylsulfonyl)imidazo [1,2-a]pyridin-7- yl)oxy)methyl) cyclopropan-1-ol | [M + H]⁺ = 453.1 ¹H NMR (400 MHz, DMSO- d₆) δ ppm 8.37 (d, J = 2.4 Hz, 1H), 7.77 (dd, J = 10.0, 7.6 Hz, 1H), 7.66 (s, 1H), 7.28 (s, 1H), 7.06 (s, 2H), 5.40 (s, 1H), 4.20 (s, 2H), 1.33 (s, 9H), 0.70-0.69 (m, 4H). |
| 178 | 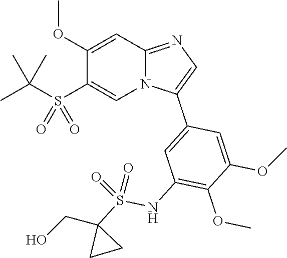 [SMILES: COc1cc2ncc(-c3cc(NS(=O)(=O)C4(CO)CC4)c(OC)c(OC)c3)n2cc1S(=O)(=O)C(C)(C)C] | N-(5-(6-(tert- butylsulfonyl)-7- methoxyimidazo[1,2- a]pyridin-3-yl)-2,3- dimethoxyphenyl)-1- (hydroxymethyl) cyclopropane-1- sulfonamide | [M + H]⁺ = 554.1 ¹H NMR (400 MHz, DMSO- d₆) δ ppm 9.09 (s, 1H), 8.73 (s, 1H), 7.73 (s, 1H), 7.30- 7.20 (m, 2H), 7.09 (d, J = 2.0 Hz, 1H), 5.07 (t, J = 6.0 Hz, 1H), 3.93 (s, 3H), 3.90-3.78 (m, 8H), 1.32 (s, 9H), 1.17- 1.03 (m, 2H), 1.05-0.90 (m, 2H). |
| 179 | 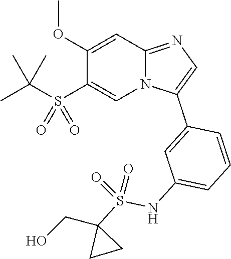 [SMILES: COc1cc2ncc(-c3cccc(NS(=O)(=O)C4(CO)CC4)c3)n2cc1S(=O)(=O)C(C)(C)C] | N-(3-(6-(tert- butylsulfonyl)-7- methoxyimidazo[1,2- a]pyridin-3- yl)phenyl)-1- (hydroxymethyl) cyclopropane-1- sulfonamide | [M + H]⁺ = 494.1 ¹H NMR (400 MHz, DMSO- d₆) δ ppm 10.09-9.64 (m, 1H), 8.70 (s, 1H), 7.73 (s, 1H), 7.57-7.45 (m, 1H), 7.44 (s, 1H), 7.36-7.24 (m, 3H), 5.19-4.65 (m, 1H), 3.94 (s, 3H), 3.78 (s, 2H), 1.32 (s, 9H), 1.11-1.02 (m, 2H), 0.99-0.87 (m, 2H). |
| 180 | 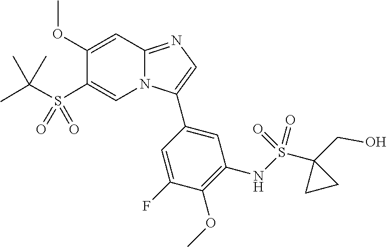 [SMILES: COc1cc2ncc(-c3cc(F)c(OC)c(NS(=O)(=O)C4(CO)CC4)c3)n2cc1S(=O)(=O)C(C)(C)C] | N-(5-(6-(tert- butylsulfonyl)-7- methoxyimidazo[1,2- a]pyridin-3-yl)-3- fluoro-2- methoxyphenyl)-1- (hydroxymethyl) cyclopropane-1- sulfonamide | [M + H]⁺ = 542.0 ¹H NMR (400 MHz, DMSO- d₆) δ ppm 9.43 (s, 1H), 8.68 (s, 1H), 7.78 (s, 1H), 7.47 (s, 1H), 7.43-7.33 (m, 1H), 7.29 (s, 1H), 5.06 (br s, 1H), 3.98- 3.91 (m, 6H), 3.82 (s, 2H), 1.32 (s, 9H), 1.13-1.07 (m, 2H), 1.01-0.95 (m, 2H). |
| 181 | 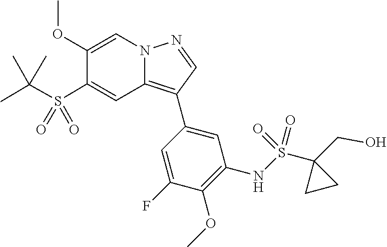 [SMILES: COc1cn2ncc(-c3cc(F)c(OC)c(NS(=O)(=O)C4(CO)CC4)c3)c2cc1S(=O)(=O)C(C)(C)C] | N-(5-(5-(tert- butylsulfonyl)-6- methoxypyrazolo[1,5- a]pyridin-3-yl)-3- fluoro-2- methoxyphenyl)-1- (hydroxymethyl) cyclopropane-1- sulfonamide | [M + H]⁺ = 542.2 ¹H NMR (400 MHz, DMSO- d₆) δ ppm 9.41-9.18 (m, 1H), 8.75 (s, 1H), 8.42 (s, 1H), 8.26 (s, 1H), 7.56 (s, 1H), 7.38 (dd, J = 12.4, 1.6 Hz, 1H), 5.04 (br s, 1H), 3.92 (s, 6H), 3.83 (s, 2H), 1.33 (s, 9H), 1.15-1.10 (m, 2H), 1.02-0.97 (m, 2H). |
| 182 | 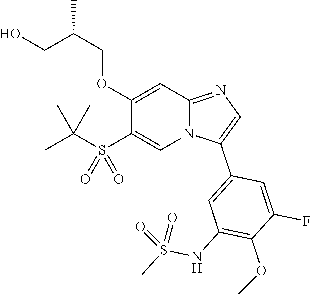 [SMILES: COc1c(F)cc(-c2cnc3cc(OC[C@@H](C)CO)c(S(=O)(=O)C(C)(C)C)cn23)cc1NS(C)(=O)=O] | (S)-N-(5-(6-(tert- butylsulfonyl)-7-(3- hydroxy-2- methylpropoxy)imidazo [1,2-a]pyridin-3- yl)-3-fluoro-2- methoxyphenyl) methanesulfonamide | [M + H]⁺ = 544.1 ¹H NMR (400 MHz, DMSO- d₆) δ ppm 9.56 (s, 1H), 8.71 (s, 1H), 7.76 (s, 1H), 7.42- 7.36 (m, 2H), 7.22 (s, 1H), 4.57 (t, J = 5.2 Hz, 1H), 4.13-4.07 (m, 1H), 4.04-3.99 (m, 1H), 3.94 (d, J = 1.6 Hz, 3H), 3.51-3.42 (m, 2H), 3.12 (s, 3H), 2.08-2.00 (m, 1H), 1.34 (s, 9H), 1.01 (d, J = 6.8 Hz, 3H). SFC: Rₜ = 1.158 min, 99.1% ee; CHIRALPAK IC-3 50 × 4.6 mm, 3 μm; 40% MeOH/MeCN (3/1, 0.05% DEA) in CO₂, 3 mL/min; 35° C., 100 Bar, 254 nm |
| 183 | 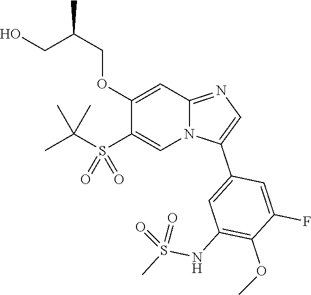 [SMILES: COc1c(F)cc(-c2cnc3cc(OC[C@H](C)CO)c(S(=O)(=O)C(C)(C)C)cn23)cc1NS(C)(=O)=O] | (R)-N-(5-(6-(tert- butylsulfonyl)-7-(3- hydroxy-2- methylpropoxy)imidazo [1,2-a]pyridin-3- yl)-3-fluoro-2- methoxyphenyl) methanesulfonamide | [M + H]⁺ = 544.0 ¹H NMR (400 MHz, DMSO- d₆) δ ppm 9.56 (br s, 1H), 8.71 (s, 1H), 7.76 (s, 1H), 7.44-7.34 (m, 2H), 7.22 (s, 1H), 4.57 (t, J = 5.2 Hz, 1H), 4.11 (dd, J = 9.6, 6.0 Hz, 1H), 4.01 (dd, J = 9.6, 6.0 Hz, 1H), 3.94 (d, J = 1.2 Hz, 3H), 3.53-3.40 (m, 2H), 3.12 (s, 3H), 2.08-1.99 (m, 1H), 1.34 (s, 9H), 1.01 (d, J = 6.8 Hz, 3H). SFC: Rₜ = 1.623 min, 94.2% ee; CHIRALPAK IC-3 50 × 4.6 mm, 3 μm; 40% MeOH/MeCN (3/1, 0.05% DEA) in CO₂, 3 mL/min; 35° C., 100 Bar, 220 nm |
| 184 | 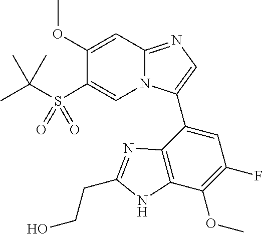 [SMILES: COc1cc2ncc(-c3cc(F)c(OC)c4[nH]c(CCO)nc34)n2cc1S(=O)(=O)C(C)(C)C] | 2-(4-(6-(tert- butylsulfonyl)-7- methoxyimidazo[1,2- a]pyridin-3-yl)-6- fluoro-7-methoxy- 1H- benzo[d]imidazol-2- yl)ethan-1-ol | [M + H]⁺ = 477.1 ¹H NMR (400 MHz, DMSO- d₆) δ ppm 13.03-12.18 (m, 1H), 9.00-8.11 (m, 1H), 7.88-7.68 (m, 1H), 7.41-7.16 (m, 2H), 4.88-4.64 (m, 1H), 4.41-4.02 (m, 3H), 3.95 (s, 3H), 3.74 (br s, 2H), 2.93- 2.83 (m, 2H), 1.36-1.27 (m, 9H). |

### Example 185

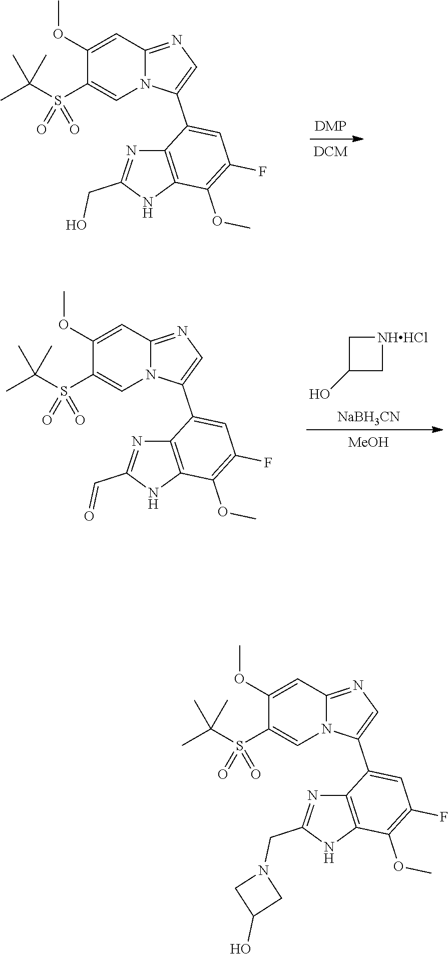

4-(6-(tert-butylsulfonyl)-7-methoxyimidazo[1,2-a]pyridin-3-yl)-6-fluoro-7-methoxy-1H-benzo[d]imidazole-2-carbaldehyde: To a solution of (4-(6-(tert-butylsulfonyl)-7-methoxyimidazo[1,2-a]pyridin-3-yl)-6-fluoro-7-methoxy-1H-benzo[d]imidazol-2-yl)methanol (35 mg, 60.54 μmol, 1 eq) in DCM (3.5 mL) was added DMP (28.14 μL, 90.81 μmol, 1.5 eq) portionwise. The mixture was stirred at 20° C. for 2 h before it was diluted with H₂O (10 mL) and extracted with DCM (3×15 mL). The combined organic layers were dried over Na₂SO₄, filtered and concentrated in vacuo. The resulting crude material was purified by column chromatography (SiO₂, 0-10% MeOH/DCM) to yield the title compound. ¹H NMR (400 MHz, DMSO-d₆) δ ppm 14.55-13.55 (m, 1H), 9.97-9.72 (m, 1H), 8.78-7.95 (m, 1H), 7.93-7.72 (m, 1H), 7.61-7.50 (m, 1H), 7.36-7.29 (m, 1H), 4.48-4.08 (m, 3H), 3.96 (s, 3H), 1.39-1.25 (m, 9H).

1-((4-(6-(tert-butylsulfonyl)-7-methoxyimidazo[1,2-a]pyridin-3-yl)-6-fluoro-7-methoxy-1H-benzo[d]imidazol-2-yl)methyl)azetidin-3-ol: A mixture of 4-(6-(tert-butylsulfonyl)-7-methoxyimidazo[1,2-a]pyridin-3-yl)-6-fluoro-7-methoxy-1H-benzo[d]imidazole-2-carbaldehyde (50 mg, 86.87 μmol, 1 eq) and 3-hydroxyazetidine hydrochloride (47.58 mg, 434.33 μmol, 5 eq) in MeOH (4.5 mL) was stirred at 25° C. for 1 h, and then NaBH₃CN (16.38 mg, 260.60 μmol, 3 eq) was added. The mixture was stirred at 25° C. for 12 h before it was concentrated in vacuo, and the resulting crude material was purified via prep RP-HPLC to yield the title compound. LCMS [M+H]⁺=518.2. ¹H NMR (400 MHz, DMSO-d₆) δ ppm 9.13-7.90 (m, 1H), 7.77 (s, 1H), 7.34-7.24 (m, 2H), 5.28 (d, J=5.6 Hz, 1H), 4.56-4.24 (m, 1H), 4.21-4.03 (m, 3H), 3.95 (s, 3H), 3.66 (s, 2H), 3.51-3.47 (m, 2H), 2.85 (t, J=6.4 Hz, 2H), 1.33 (s, 9H).

### Example 186

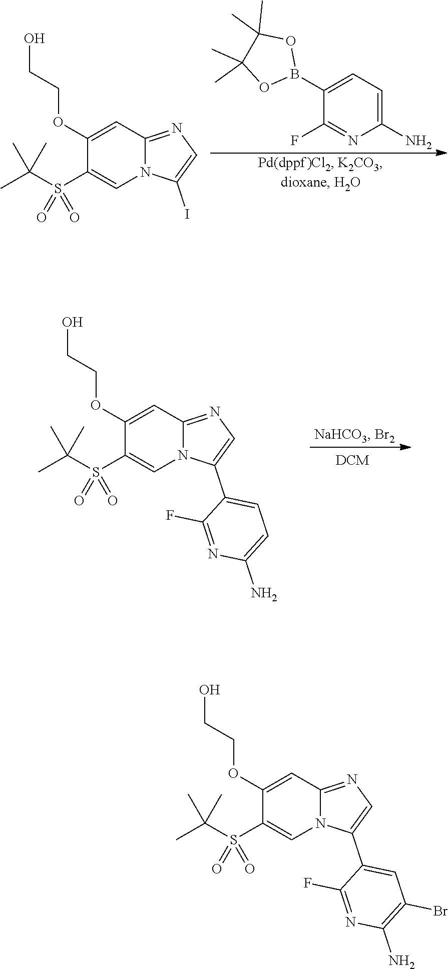

2-((3-(6-amino-2-fluoropyridin-3-yl)-6-(tert-butylsulfonyl)imidazo[1,2-a]pyridin-7-yl)oxy)ethan-1-ol: To a mixture of 6-fluoro-5-(4,4,5,5-tetramethyl-1,3,2-dioxaborolan-2-yl)pyridin-2-amine (100 mg, 221.57 μmol, 1 eq), 2-((6-(tert-butylsulfonyl)-3-iodoimidazo[1,2-a]pyridin-7-yl)oxy)ethan-1-ol (73.85 mg, 310.19 μmol, 1.4 eq) and K₂CO₃ (91.86 mg, 664.70 μmol, 3 eq) in 1,4-dioxane (10 mL) and H₂O (2 mL) was added Pd(dppf)Cl₂ (16.21 mg, 22.16 μmol, 0.1 eq). The mixture was degassed and purged with N₂ 3 times, and then the mixture was stirred at 100° C. for 3 hours before it was diluted with H₂O (5 mL) and extracted with EtOAc (3×10 mL). The combined organic layers were washed with brine (2×10 mL), dried over MgSO₄, filtered and concentrated under reduced pressure to give a residue, which was purified by RP-HPLC to yield the title compound (35 mg, 37% yield). [M+H]⁺=409.1. ¹H NMR (400 MHz, CDCl₃) δ ppm 8.48 (s, 1H), 7.64-7.59 (m, 2H), 7.12 (s, 1H), 6.51 (d, J=7.6 Hz, 1H), 4.81 (s, 2H), 4.29 (t, J=3.5 Hz, 2H), 4.01-3.96 (m, 2H), 1.46 (s, 9H).

2-((3-(6-amino-5-bromo-2-fluoropyridin-3-yl)-6-(tert-butylsulfonyl)imidazo[1,2-a]pyridin-7-yl)oxy)ethan-1-ol: To a solution of 2-((3-(6-amino-2-fluoropyridin-3-yl)-6-(tert-butylsulfonyl)imidazo[1,2-a]pyridin-7-yl)oxy)ethan-1-ol (14 mg, 32.91 μmol, 1 eq) and NaHCO₃ (2.76 mg, 32.91 μmol, 1.28 L, 1 eq) in DCM (0.3 mL) was added Br2 (3.68 mg, 23.03 μmol, 1.19 μL, 0.7 eq) at 0° C. The mixture was stirred at 0° C. for 1 hour before it was quenched with sat. NaHCO₃ (5 mL) at 0° C. and extracted with EtOAc (3×10 mL). The combined organic layers were washed with brine (2×5 mL), dried over MgSO₄, filtered and concentrated under reduced pressure to give a residue, which was purified by RP-HPLC to yield the title compound (3.94 mg, 24% yield) as a white solid. [M+H]⁺=488.8. ¹H NMR (400 MHz, CDCl₃) δ ppm 8.46 (d, J=2.6 Hz, 1H), 7.83 (d, J=8.3 Hz, 1H), 7.63 (s, 1H), 7.06 (d, J=2.3 Hz, 1H), 5.25 (s, 2H), 4.29 (t, J=4.3 Hz, 2H), 4.01-3.97 (m, 2H), 3.30-3.20 (m, 1H), 1.46 (s, 9H).

### Example 187

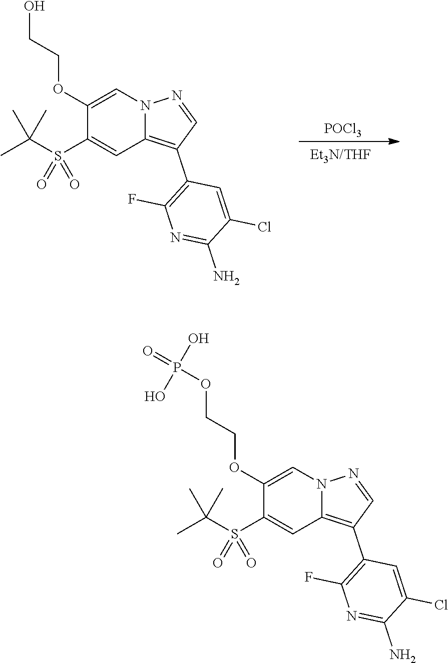 [SMILES: CC(C)(C)S(=O)(=O)c1cc2c(-c3cc(Cl)c(N)nc3F)cnn2cc1OCCO ; "POCl3" ; "Et3N/THF" ; CC(C)(C)S(=O)(=O)c1cc2c(-c3cc(Cl)c(N)nc3F)cnn2cc1OCCOP(=O)(O)O]

2-((3-(6-amino-5-chloro-2-fluoropyridin-3-yl)-5-(tert-butylsulfonyl)pyrazolo[1,5-a]pyridin-6-yl)oxy)ethyl dihydrogen phosphate: To a mixture of 2-((3-(6-amino-5-chloro-2-fluoropyridin-3-yl)-5-(tert-butylsulfonyl)pyrazolo[1,5-a]pyridin-6-yl)oxy)ethan-1-ol (60 mg, 108.38 μmol, 1 eq) in Et₃N (0.2 mL) and THF (1 mL) was added POCl₃ (166.18 mg, 1.08 mmol, 101.02 L, 10 eq) dropwise slowly at 0° C. with vigorous stirring. The mixture was stirred at 0° C. for 2 hours before it was neutralized with sat. NaHCO₃ solution and washed with DCM to remove impurities. The aqueous phase was acidified with 1 M HCl (pH=2) and lyophilized to give a crude product, which was purified by RP-HPLC to give the title compound (15.7 mg, 27% yield) as a yellow solid. [M+H]⁺=523.2. ¹H NMR (400 MHz, DMSO-d₆) δ ppm 8.78 (s, 1H), 8.30 (s, 1H), 8.11 (s, 1H), 7.97 (d, J=8.8 Hz, 1H), 6.88 (s, 2H), 4.35-4.27 (m, 2H), 4.19-4.12 (m, 2H), 1.32 (s, 9H).

### Example 188, 2-((3-(6-amino-2,5-difluoropyridin-3-yl)-5-(tert-butylsulfonyl)pyrazolo[1,5-a]pyridin-6-yl)oxy)ethyl dihydrogen phosphate, was prepared following procedures analogous to that described for Example 187

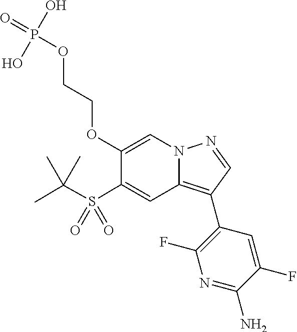 [SMILES: CC(C)(C)S(=O)(=O)c1cc2c(-c3cc(F)c(N)nc3F)cnn2cc1OCCOP(=O)(O)O]

[M+H]⁺=507.2

¹H NMR (400 MHz, DMSO-d₆) δ ppm 8.77 (s, 1H), 8.28 (s, 1H), 8.12 (s, 1H), 7.81 (dd, J=10.8, 7.6 Hz, 1H), 6.81 (s, 2H), 4.32-4.28 (m, 2H), 4.18-4.09 (m, 2H), 1.32 (s, 9H).

### Example 189

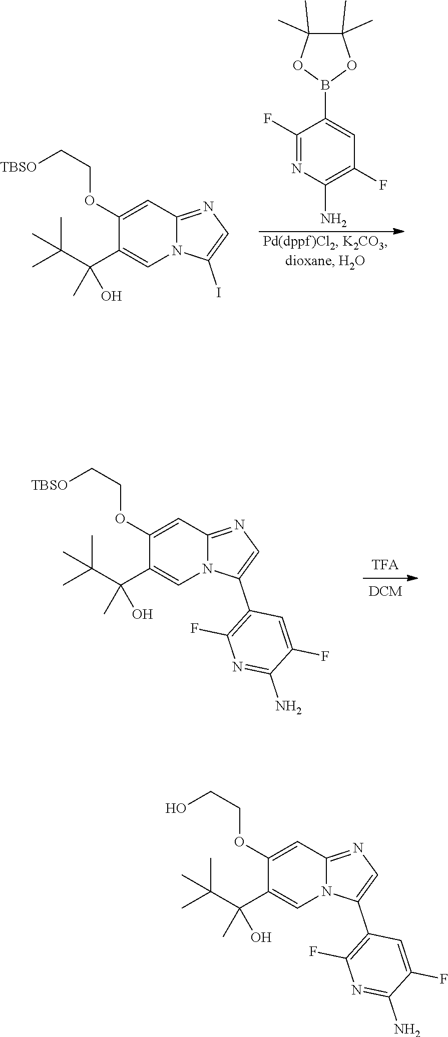

2-(3-(6-amino-2,5-difluoropyridin-3-yl)-7-(2-((tert-butyldimethylsilyl)oxy)ethoxy)imidazo[1,2-a]pyridin-6-yl)-3,3-dimethylbutan-2-ol: A mixture of 2-(7-(2-((tert-butyldimethylsilyl)oxy)ethoxy)-3-iodoimidazo[1,2-a]pyridin-6-yl)-3,3-dimethylbutan-2-ol (100 mg, 173.58 μmol, 1 eq), 3,6-difluoro-5-(4,4,5,5-tetramethyl-1,3,2-dioxaborolan-2-yl)pyridin-2-amine (74.08 mg, 260.37 μmol, 1.5 eq), Pd(dppf)Cl₂ (12.70 mg, 17.36 μmol, 0.1 eq) and K₂CO₃ (71.97 mg, 520.73 μmol, 3 eq) in dioxane (5 mL) and H₂O (1 mL) was degassed and purged with N₂ for 3 times, and then the mixture was stirred at 80° C. for 2 hours under N₂ atmosphere. The reaction mixture was concentrated under reduced pressure to remove solvent. The residue was purified by column chromatography (SiO₂, 0-70% EtOAc in PE) to yield the title compound (100 mg, 99% yield) as a brown solid. ¹H NMR (400 MHz, DMSO-d₆) δ ppm 8.11 (s, 1H), 7.72-7.63 (m, 1H), 7.45 (s, 1H), 6.95 (s, 3H), 4.87 (s, 1H), 4.14-4.08 (m, 1H), 4.06-4.01 (m, 1H), 3.98-3.92 (m, 2H), 1.66 (s, 3H), 0.90-0.86 (m, 18H), 0.09 (s, 6H).

2-(3-(6-amino-2,5-difluoropyridin-3-yl)-7-(2-hydroxyethoxy)imidazo[1,2-a]pyridin-6-yl)-3,3-dimethylbutan-2-ol: To a solution of 2-(3-(6-amino-2,5-difluoropyridin-3-yl)-7-(2-((tert-butyldimethylsilyl)oxy)ethoxy)imidazo[1,2-a]pyridin-6-yl)-3,3-dimethylbutan-2-ol (10 mg, 17.28 μmol, 1 eq) in DCM (2 mL) was added TFA (614.00 mg, 5.38 mmol, 400.00 μL, 311.54 eq). The mixture was stirred at 25° C. for 3 hours. The reaction mixture was concentrated under reduced pressure to remove solvent. The residue was purified by RP-HPLC to yield the title compound (3.33 mg, 4700 yield) as a yellow solid. [M+H]⁺=407.1. ¹H₉NMR (400 MHz, DMSO-d₆) δ ppm 8.11 (s, 1H), 7.72-7.64 (m, 1H), 7.46 (s, 1H), 6.95 (d, JH 2.0 Hz, 3H), 4.91-4.83 (i, 2H), 4.11-4.05 (m, 1H), 4.03-3.96 (m, 1H), 3.79-3.74 (, 2H), 1.65 (s, 3H), 0.88 (s, 9H).

The following intermediate compounds (denoted as =nt.) were prepared following procedures analogous to that described for Intermediate 70. The following compounds were prepared following procedures analogous to that described above. Examples prepared using intermediates or building blocks containing Boc, T-P, TBS, dioxolane or other similar protecting groups were subjected to protecting group removal under acidic conditions (generally with HCl or TFA) prior to purification of the final product, as appropriate.

| Ex. | | | |
|---|---|---|---|
| No. | Structure | Name | LCMS, ¹H NMR |
| 190 | 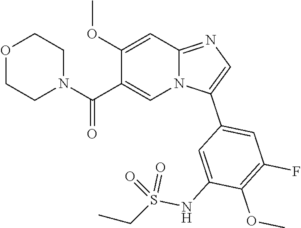 [SMILES: CCS(=O)(=O)Nc1cc(-c2cnc3cc(OC)c(C(=O)N4CCOCC4)cn23)cc(F)c1OC] | N-(3-fluoro-2- methoxy-5-(7- methoxy-6- (morpholine-4- carbonyl)imidazo[1,2- a]pyridin-3- yl)phenyl) ethanesulfonamide | [M + H]⁺ = 493 ¹H NMR: ES20817- 658-P1B (400 MHz, DMSO-d₆) δ ppm: 9.54 (s, 1H), 8.36 (s, 1H), 7.64 (s, 1H), 7.38-7.33 (m, 2H), 7.14 (s, 1H), 3.92 (d, J = 1.6 Hz, 3H), 3.89 (s, 3H), 3.65- 3.49 (m, 6H), 3.26-3.15 (m, 4H), 1.27 (t, J = 7.6 Hz, 3H). |
| Int. 147 | 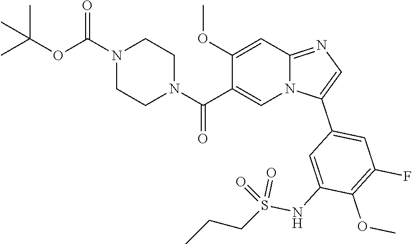 [SMILES: CCCS(=O)(=O)Nc1cc(-c2cnc3cc(OC)c(C(=O)N4CCN(C(=O)OC(C)(C)C)CC4)cn23)cc(F)c1OC] | tert-butyl 4-(3-(3- fluoro-4-methoxy-5- (propylsulfonamido) phenyl)-7- methoxyimidazo[1,2- a]pyridine-6- carbonyl)piperazine- 1-carboxylate | ¹H NMR: (400 MHz, CDCl₃) δ ppm: 8.24 (s, 1H), 7.59 (s, 1H), 7.46 (s, 1H), 7.07 (s, 1H), 7.04-6.98 (m, 2H), 4.09 (d, J = 2.8 Hz, 3H), 3.94 (s, 3H), 3.55-3.39 (m, 6H), 3.34-3.26 (m, 2H), 3.12-3.07 (m, 2H), 1.88-1.85 (m, 2H), 1.24 (s, 9H), 1.05 (t, J = 7.6 Hz, 3H). |
| 191 | 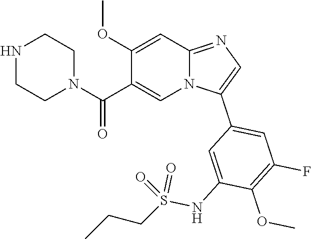 [SMILES: CCCS(=O)(=O)Nc1cc(-c2cnc3cc(OC)c(C(=O)N4CCNCC4)cn23)cc(F)c1OC] | N-(3-fluoro-2- methoxy-5-(7- methoxy-6- (piperazine-1- carbonyl)imidazo[1,2- a]pyridin-3- yl)phenyl)propane-1- sulfonamide | [M + H] ⁺ = 506.1 ¹H NMR: (400 MHz, DMSO-d₆) δ ppm: 8.31 (s, 1H), 7.64 (s, 1H), 7.38-7.29 (m, 2H), 7.13 (s, 1H), 3.91 (d, J = 1.2 Hz, 3H), 3.89 (s, 3H), 3.56-3.50 (m, 2H), 3.15 (t, J = 7.6 Hz, 4H), 2.73 (s, 2H), 2.65-2.59 (m, 2H), 1.81-1.70 (m, 2H), 0.97 (t, J = 7.6 Hz, 3H). |
| 192 | 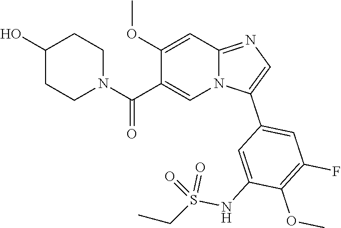 [SMILES: CCS(=O)(=O)Nc1cc(-c2cnc3cc(OC)c(C(=O)N4CCC(O)CC4)cn23)cc(F)c1OC] | N-(3-fluoro-5-(6-(4- hydroxypiperidine-1- carbonyl)-7- methoxyimidazo[1,2- a]pyridin-3-yl)-2- methoxyphenyl) ethanesulfonamide | [M + H] ⁺ = 507.1 ¹H NMR: (400 MHz, DMSO-d₆) δ ppm: 8.32 (d, J = 9.6 Hz, 1H), 8.18 (s, 0.2H), 7.64 (s, 1H), 7.45-7.30 (m, 2H), 7.13 (s, 1H), 4.76 (br s, 1H), 4.05-3.95 (m, 1H), 3.92 (s, 3H), 3.88 (d, J = 5.6 Hz, 3H), 3.72 (s, 1H), 3.30-3.28 (m, 1H), 3.21-3.01 (m, 4H), 1.82-1.60 (m, 2H), 1.43-1.31 (m, 2H), 1.26 (t, J = 7.2 Hz, 3H). |
| Int. 148 | 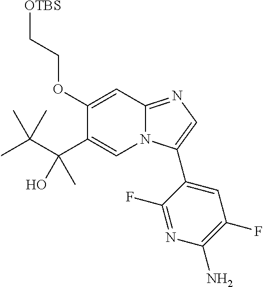 [SMILES: CC(C)(C)C(C)(O)c1cn2c(-c3cc(F)c(N)nc3F)cnc2cc1OCCO[Si](C)(C)C(C)(C)C] | 2-(3-(6-amino-2,5- difluoropyridin-3-yl)- 7-(2-((tert- butyldimethylsilyl)ox y)ethoxy)imidazo[1,2- a]pyridin-6-yl)-3,3- dimethylbutan-2-ol | ¹H NMR: (400 MHz, DMSO-d₆) δ ppm: 8.11 (s, 1H), 7.72-7.63 (m, 1H), 7.45 (s, 1H), 6.95 (s, 3H), 4.87 (s, 1H), 4.14-4.08 (m, 1H), 4.06-4.01 (m, 1H), 3.98-3.92 (m, 2H), 1.66 (s, 3H), 0.90 -0.86 (m, 18H), 0.09 (s, 6H). |
| 193 | 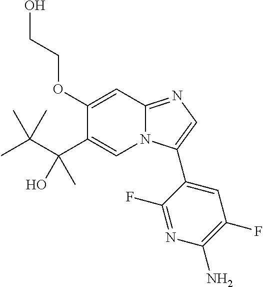 [SMILES: CC(C)(C)C(C)(O)c1cn2c(-c3cc(F)c(N)nc3F)cnc2cc1OCCO] | 2-(3-(6-amino-2,5- difluoropyridin-3-yl)- 7-(2-hydroxyethoxy) imidazo[1,2-a] pyridin-6-yl)-3,3- dimethylbutan-2-ol | [M + H] ⁺ = 407.1 ¹H NMR: (400 MHz, DMSO-d₆) δ ppm: 8.11 (s, 1H), 7.72-7.64 (m, 1H), 7.46 (s, 1H), 6.95 (d, J = 2.0 Hz, 3H), 4.91-4.83 (m, 2H), 4.11-4.05 (m, 1H), 4.03-3.96 (m, 1H), 3.79-3.74 (m, 2H), 1.65 (s, 3H), 0.88 (s, 9H). |
| 194 | 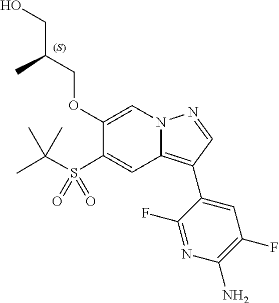 | (S)-3-((3-(6-amino- 2,5-difluoropyridin-3- yl)-5-(tert- butylsulfonyl)pyrazolo [1,5-a]pyridin-6- yl)oxy)-2- methylpropan-1-ol | [M + H] ⁺ = 455.1 ¹H NMR: (400 MHz, DMSO-d₆) δ ppm 8.66 (s, 1H), 8.43 (s, 0.3H), 8.26 (s, 1H), 8.10 (d, J = 1.6 Hz, 1H), 7.80 (d, J = 7.6 Hz, 1H), 6.79 (s, 2H), 4.56 (s, 1H), 4.08-3.94 (m, 2H), 3.51-3.41 (m, 2H), 2.03 (d, J = 6.4 Hz, 1H), 1.31 (s, 9H), 1.01 (d, J = 6.8 Hz, 3H). |
| 195 | 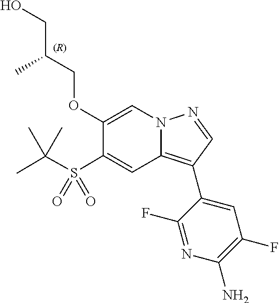 | (R)-3-((3-(6-amino- 2,5-difluoropyridin-3- yl)-5-(tert- butylsulfonyl)pyrazolo [1,5-a]pyridin-6- yl)oxy)-2- methylpropan-1-ol | [M + H] ⁺ = 455.1 ¹H NMR: (400 MHz, DMSO-d₆) δ ppm 8.66 (s, 1H), 8.26 (s, 1H), 8.10 (d, J = 1.6 Hz, 1H), 7.80 (d, J = 7.6 Hz, 1H), 6.79 (s, 2H), 4.56 (t, J = 5.2 Hz, 1H), 4.07-3.95 (m, 2H), 3.51-3.41 (m, 2H), 2.08-1.98 (m, 1H), 1.31 (s, 9H), 1.01 (d, J = 6.8 Hz, 3H). |
| 196 | 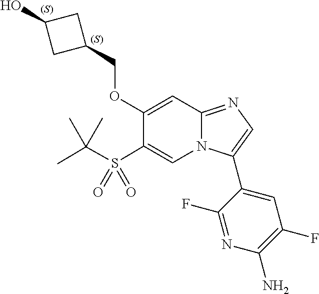 | (1s,3s)-3-(((3-(6- amino-2,5- difluoropyridin-3-yl)- 6-(tert- butylsulfonyl)imidazo [1,2-a]pyridin-7- yl)oxy)methyl) cyclobutan-1-ol | [M + H] ⁺ = 467.1 ¹H NMR: (400 MHz, DMSO-d₆) δ ppm: 8.36 (s, 1H), 7.76 (dd, J = 7.2, 10.0 Hz, 1H), 7.66 (s, 1H), 7.22 (s, 1H), 7.05 (s, 2H), 5.01 (d, J = 6.4 Hz, 1H), 4.09 (d, J = 6.4 Hz, 2H), 4.07- 3.96 (m, 1H), 2.39-2.25 (m, 2H), 2.27-2.10 (m, 1H), 1.79-1.60 (m, 2H), 1.31 (s, 9H). |
| 197 | 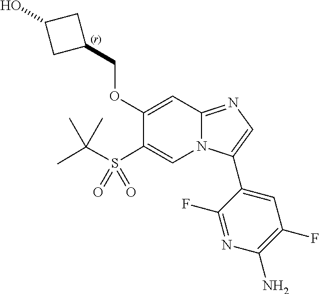 | (1r,3r)-3-(((3-(6- amino-2,5- difluoropyridin-3-yl)- 6-(tert- butylsulfonyl)imidazo [1,2-a]pyridin-7- yl)oxy)methyl) cyclobutan-1-ol | [M + H] ⁺ = 467.1 ¹H NMR: (400 MHz, DMSO-d₆) δ ppm: 8.36 (s, 1H), 7.77 (dd, J = 7.6, 10.4 Hz, 1H), 7.66 (s, 1H), 7.24 (s, 1H), 7.06 (s, 2H), 5.02 (d, J = 6.0 Hz, 1H), 4.40- 4.25 (m, 1H), 4.15 ( d, J = 7.2 Hz, 2H), 2.60- 2.50 (m, 1H), 2.20-2.10 (m, 2H), 2.07-1.93 (m, 2H), 1.30 (s, 9H). |
| 198 | 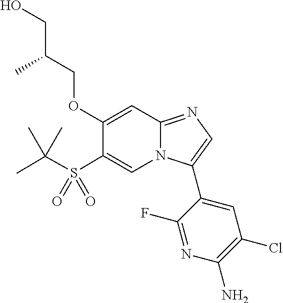 [SMILES: C[C@H](CO)COc1cc2ncc(-c3cc(Cl)c(N)nc3F)n2cc1S(=O)(=O)C(C)(C)C] | (R)-3-((3-(6-amino-5- chloro-2- fluoropyridin-3-yl)-6- (tert- butylsulfonyl)imidazo [1,2-a]pyridin-7- yl)oxy)-2- methylpropan-1-ol | [M + H] ⁺ = 471.0 ¹H NMR: (400 MHz, DMSO-d₆) δ ppm: 8.36 (d, J = 2.4 Hz, 1H), 7.93 (d, J = 8.4 Hz, 1H), 7.68 (s, 1H), 7.21 (s, 1H), 7.12 (s, 2H), 4.57 (t, J = 5.2 Hz, 1H), 4.12-3.98 (m, 2H), 3.52-3.40 (m, 2H), 2.07-1.99 (m, 1H), 1.31 (s, 9H), 1.01 (d, J = 6.8 Hz, 3H). |
| 199 | 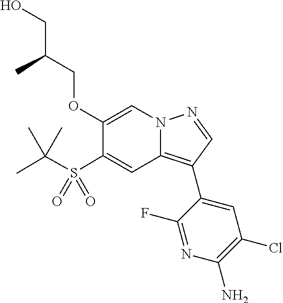 [SMILES: C[C@@H](CO)COc1cn2ncc(-c3cc(Cl)c(N)nc3F)c2cc1S(=O)(=O)C(C)(C)C] | (S)-3-((3-(6-amino-5- chloro-2- fluoropyridin-3-yl)-5- (tert- butylsulfonyl)pyrazolo [1,5-a]pyridin-6- yl)oxy)-2- methylpropan-1-ol | [M + H] ⁺ = 471.0 ¹H NMR: (400 MHz, DMSO-d₆) δ ppm: 8.66 (s, 1H), 8.28 (s, 1H), 8.09 (d, J = 1.6 Hz, 1H), 7.96 (d, J = 8.4 Hz, 1H), 6.87 (s, 2H), 4.56 (t, J = 5.2 Hz, 1H), 4.08-3.96 (m, 2H), 3.53-3.40 (m, 2H), 2.07-1.98 (m, 1H), 1.31 (s, 9H), 1.01 (d, J = 6.8 Hz, 3H). |
| 200 | 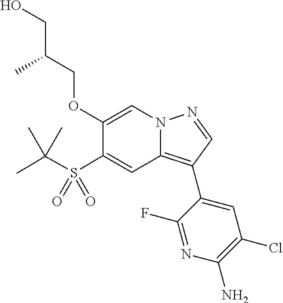 [SMILES: C[C@H](CO)COc1cn2ncc(-c3cc(Cl)c(N)nc3F)c2cc1S(=O)(=O)C(C)(C)C] | (R)-3-((3-(6-amino-5- chloro-2- fluoropyridin-3-yl)-5- (tert- butylsulfonyl)pyrazolo [1,5-a]pyridin-6- yl)oxy)-2- methylpropan-1-ol | [M + H] ⁺ = 471.0 ¹H NMR: (400 MHz, DMSO-d₆) δ ppm: 8.66 (s, 1H), 8.28 (s, 1H), 8.09 (d, J = 1.6 Hz, 1H), 7.96 (d, J = 8.8 Hz, 1H), 6.87 (s, 2H), 4.56 (t, J = 5.2 Hz, 1H), 4.08-3.96 (m, 2H), 3.52-3.41 (m, 2H), 2.08-1.99 (m, 1H), 1.31 (s, 9H), 1.01 (d, J = 6.8 Hz, 3H). |
| 201 | 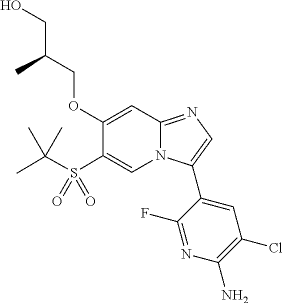 [SMILES: C[C@@H](CO)COc1cc2ncc(-c3cc(Cl)c(N)nc3F)n2cc1S(=O)(=O)C(C)(C)C] | (S)-3-((3-(6-amino-5- chloro-2- fluoropyridin-3-yl)-6- (tert- butylsulfonyl)imidazo [1,2-a]pyridin-7- yl)oxy)-2- methylpropan-1-ol | [M + H] ⁺ = 471.0 ¹H NMR: (400 MHz, DMSO-d₆) δ ppm: 8.36 (d, J = 2.0 Hz, 1H), 7.93 (d, J = 8.4 Hz, 1H), 7.68 (s, 1H), 7.21 (s, 1H), 7.11 (br s, 2H), 4.56 (t, J = 4.8 Hz, 1H), 4.14-4.06 (m, 1H), 4.04-3.97 (m, 1H), 3.54-3.39 (m, 2H), 2.08-1.99 (m, 1H), 1.31 (s, 9H), 1.01 (d, J = 6.8 Hz, 3H). |
| 202 | 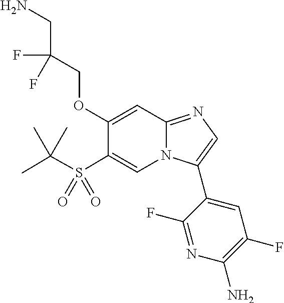 [SMILES: CC(C)(C)S(=O)(=O)c1cn2c(-c3cc(F)c(N)nc3F)cnc2cc1OCC(F)(F)CN] | 5-(7-(3-amino-2,2- difluoropropoxy)-6- (tert- butylsulfonyl)imidazo [1,2-a]pyridin-3-yl)- 3,6-difluoropyridin-2- amine | [M + H] ⁺ = 476.0. ¹H NMR: (400 MHz, DMSO-d₆) δ ppm: 8.39 (d, J = 2.4 Hz, 1H), 7.77 (dd, J = 7.6, 10.4 Hz, 1H), 7.70 (s, 1H), 7.40 (s, 1H), 7.07 (s, 2H), 4.52 (t, J = 12.4 Hz, 2H), 3.11 (t, J = 14.4 Hz, 2H), 1.73 (s, 2H), 1.30 (s, 9H). |
| Int. 149 | 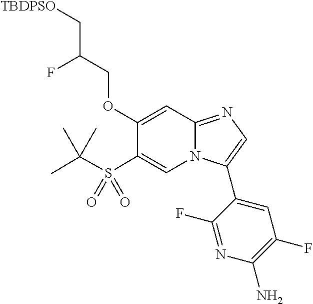 | 5-(7-(3-((tert- butyldiphenylsilyl) oxy)-2-fluoropropoxy)- 6-(tert- butylsulfonyl)imidazo [1,2-a]pyridin-3-yl)- 3,6-difluoropyridin-2- amine | [M + H] ⁺ = 697.4 ¹H NMR: (400 MHz, DMSO-d₆) δ ppm: 8.35 (s, 1H), 7.82-7.72 (m, 1H), 7.71-7.61 (m, 5H), 7.50-7.39 (m, 6H), 7.28 (s, 1H), 7.06 (s, 2H), 5.14-4.93 (m, 1H), 4.54-4.32 (m, 2H), 4.18-4.02 (m, 2H), 1.24 (s, 9H), 1.02 (s, 9H). |
| 203 | 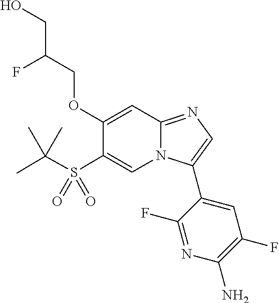 [SMILES: CC(C)(C)S(=O)(=O)c1cn2c(-c3cc(F)c(N)nc3F)cnc2cc1OCC(F)CO] | 3-((3-(6-amino-2,5- difluoropyridin-3-yl)- 6-(tert- butylsulfonyl)imidazo [1,2-a]pyridin-7- yl)oxy)-2- fluoropropan-1-ol | [M + H] ⁺ = 459.0 ¹H NMR: (400 MHz, DMSO-d₆) δ ppm: 8.41 (s, 0.15H), 8.38 (d, J = 2.4 Hz, 1H), 7.77 (dd, J = 7.2, 10.4 Hz, 1H), 7.68 (s, 1H), 7.27 (s, 1H), 7.05 (s, 2H), 5.12- 5.02(m, 1H), 4.94-4.74 (m, 1H), 4.46-4.28 (m, 2H), 3.82-3.68 (m, 2H), 1.31 (s, 9H). |
| Int. 150 | 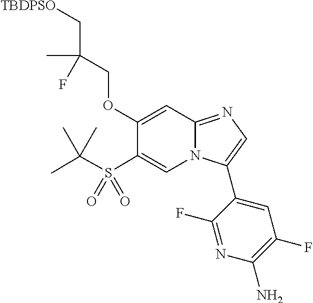 | 5-(7-(3-((tert- butyldiphenylsilyl) oxy)-2-fluoro-2- methylpropoxy)-6- (tert- butylsulfonyl)imidazo [1,2-a]pyridin-3-yl)- 3,6-difluoropyridin-2- amine | ¹H NMR: (400 MHz, DMSO-d₆) δ ppm: 8.36 (d, J = 2.4 Hz, 1H), 7.76 (dd, J = 7.6, 10.4 Hz, 1H), 7.68 (s, 1H), 7.64 (d, J = 6.4 Hz, 4H), 7.46-7.39 (m, 6H), 7.29 (s, 1H), 7.06 (s, 2H), 4.41-4.27 (m, 2H), 4.06-3.98 (m, 1H), 3.95-3.87 (m, 1H), 1.54-1.47 (m, 3H), 1.23 (s, 9H), 1.01 (s, 9H). |
| 204 | 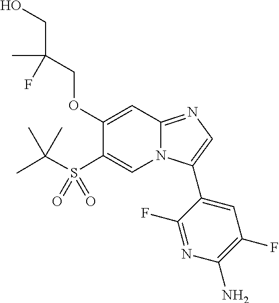 [SMILES: CC(F)(CO)COc1cc2ncc(-c3cc(F)c(N)nc3F)n2cc1S(=O)(=O)C(C)(C)C] | 3-((3-(6-amino-2,5- difluoropyridin-3-yl)- 6-(tert- butylsulfonyl)imidazo [1,2-a]pyridin-7- yl)oxy)-2-fluoro-2- methylpropan-1-ol | [M + H] ⁺ = 473.0 ¹H NMR: (400 MHz, DMSO-d₆) δ ppm: 8.37 (d, J = 2.0 Hz, 1H), 7.79-7.74 (m, 1H), 7.68 (s, 1H), 7.26 (s, 1H), 7.05 (s, 2H), 5.09 (s, 1H), 4.28-4.21 (m, 2H), 3.68-3.60 (m, 2H), 1.46-1.39 (m, 3H), 1.30 (s, 9H). |
| 205 | 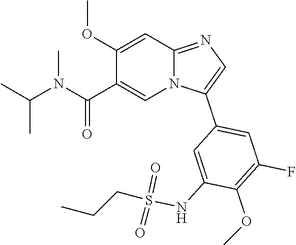 [SMILES: CCCS(=O)(=O)Nc1cc(-c2cnc3cc(OC)c(C(=O)N(C)C(C)C)cn23)cc(F)c1OC] | 3-(3-fluoro-4- methoxy-5- (propylsulfonamido) phenyl)-N-isopropyl-7- methoxy-N- methylimidazo[1,2- a]pyridine-6- carboxamide | [M + H] ⁺ = 493.1 ¹H NMR: (400 MHz, DMSO-d₆) δ ppm: 8.29 (d, J = 13.2 Hz, 1H), 7.63 (s, 1H), 7.35 (s, 1H), 7.32-7.21 (m, 1H), 7.14 (d, J = 3.2 Hz, 1H), 4.77-4.70 (m, 1H), 3.89 (d, J = 8.4 Hz, 6H), 3.76-3.69 (m, 1H), 3.14-3.09 (m, 2H), 2.82 (s, 2H), 2.66 (s, 1H), 1.78-1.69 (m, 2H), 1.15-1.05 (m, 6H), 0.98-0.94 (m, 3H). |
| 206 | 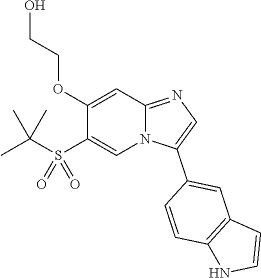 [SMILES: CC(C)(C)S(=O)(=O)c1cn2c(-c3ccc4[nH]ccc4c3)cnc2cc1OCCO] | 2-((6-(tert- butylsulfonyl)-3-(1H- indol-5- yl)imidazo[1,2- a]pyridin-7- yl)oxy)ethan-1-ol | [M + H] ⁺ = 414.0 ¹H NMR: (400 MHz, DMSO-d₆) δ ppm: 11.37 (s, 1H), 8.72 (s, 1H), 7.75 (s, 1H), 7.66 (s, 1H), 7.60 (d, J = 8.4 Hz, 1H), 7.47 (t, J = 2.4 Hz, 1H), 7.30-7.24 (m, 2H), 6.53 (s, 1H), 4.82 (s, 1H), 4.18 (t, J = 4.8 Hz, 2H), 3.78 (t, J = 4.4 Hz, 2H), 1.32 (s, 9H). |
| 207 | 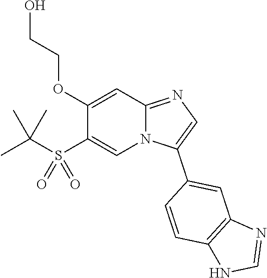 [SMILES: CC(C)(C)S(=O)(=O)c1cn2c(-c3ccc4[nH]cnc4c3)cnc2cc1OCCO] | 2-((3-(1H- benzo[d]imidazol-5- yl)-6-(tert- butylsulfonyl)imidazo [1,2-a]pyridin-7- yl)oxy)ethan-1-ol | [M + H] ⁺ = 415.0 ¹H NMR: (400 MHz, DMSO-d₆) δ ppm: 12.89-12.53 (m, 1H), 8.75 (s, 1H), 8.34 (s, 1H), 7.86-7.75 (m, 2H), 7.73 (s, 1H), 7.41 (dd, J = 1.2, 8.4 Hz, 1H), 7.28 (s, 1H), 4.82 (s, 1H), 4.19 (t, J = 4.8 Hz, 2H), 3.83-3.74 (m, 2H), 1.33 (s, 9H). |
| 208 | 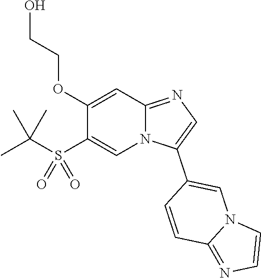 [SMILES: CC(C)(C)S(=O)(=O)c1cn2c(-c3ccc4nccn4c3)cnc2cc1OCCO] | 2-((6-(tert- butylsulfonyl)-[3,6′- biimidazo[1,2- a]pyridin] -7- yl)oxy)ethan-1-ol | [M + H] ⁺ = 415.0 ¹H NMR: (400 MHz, DMSO-d₆) δ ppm: 8.85 (s, 1H), 8.61 (s, 1H), 8.05 (s, 1H), 7.83-7.73 (m, 2H), 7.68 (s, 1H), 7.46 (d, J = 10.0 Hz, 1H), 7.31 (s, 1H), 4.82 (t, J = 5.2 Hz, 1H), 4.20 (t, J = 4.8 Hz, 2H), 3.81-3.75 (m, 2H), 1.32 (s, 9H). |
| 209 | 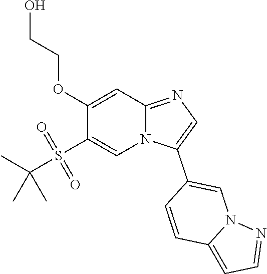 [SMILES: CC(C)(C)S(=O)(=O)c1cn2c(-c3ccc4ccnn4c3)cnc2cc1OCCO] | 2-((6-(tert- butylsulfonyl)-3- (pyrazolo[1,5- a]pyridin-6- yl)imidazo[1,2- a]pyridin-7- yl)oxy)ethan-1-ol | [M + H] ⁺ = 415.0 ¹H NMR: (400 MHz, DMSO-d₆) δ ppm: 9.02 (s, 1H), 8.65 (s, 1H), 8.11 (d, J = 2.0 Hz, 1H), 7.90 (d, J = 8.8 Hz, 1H), 7.83 (s, 1H), 7.44 (dd, J = 1.2, 9.2 Hz, 1H), 7.31 (s, 1H), 6.74 (d, J = 1.6 Hz, 1H), 4.82 (s, 1H), 4.20 (t, J = 4.8 Hz, 2H), 3.78 (s, 2H), 1.33 (s, 9H). |
| 210 | 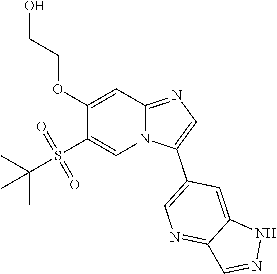 [SMILES: CC(C)(C)S(=O)(=O)c1cn2c(-c3cnc4cn[nH]c4c3)cnc2cc1OCCO] | 2-((6-(tert- butylsulfonyl)-3-(1H- pyrazolo[ 4,3- b]pyridin-6- yl)imidazo[1,2- a]pyridin-7- yl)oxy)ethan-1-ol | [M + H] ⁺ = 416.1 ¹H NMR: (400 MHz, DMSO-d₆) δ ppm: 13.57 (s, 1H), 8.82- 8.70 (m, 2H), 8.40 (s, 1H), 8.31 (s, 0.2H), 8.26 (s, 1H), 7.93 (s, 1H), 7.33 (s, 1H), 4.82 (br s, 1H), 4.21 (t, J = 4.8 Hz, 2H), 3.79 (t, J = 4.8 Hz, 2H), 1.34 (s, 9H). |
| 211 | 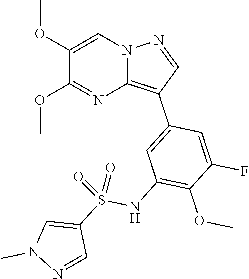 [SMILES: COc1cn2ncc(-c3cc(F)c(OC)c(NS(=O)(=O)c4cnn(C)c4)c3)c2nc1OC] | N-(5-(5,6- dimethoxypyrazolo[1, 5-a]pyrimidin-3-yl)- 3-fluoro-2- methoxyphenyl)-1- methyl-1H-pyrazole- 4-sulfonamide | [M + H] ⁺ = 463.0 ¹H NMR (400 MHz, DMSO-d₆) δ ppm: 10.20-9.21 (m, 1H), 8.71 (s, 1H), 8.37 (s, 1H), 8.16 (s, 1H), 8.10 (s, 1H), 7.69 (s, 1H), 7.58 (d, J = 12.4 Hz, 1H), 4.09 (s, 3H), 3.87 (s, 3H), 3.80 (s, 3H), 3.66 (s, 3H). |
| 212 | 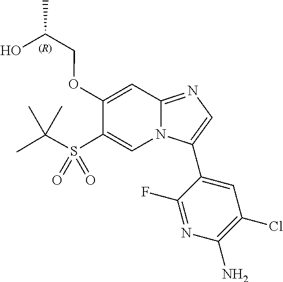 | (R)-1-((3-(6-amino-5- chloro-2- fluoropyridin-3-yl)-6- (tert- butylsulfonyl)imidazo [1,2-a]pyridin-7- yl)oxy)propan-2-ol | [M + H]⁺ = 457.0 ¹H NMR (400 MHz, DMSO-d₆) δ ppm: 8.36 (d, J = 2.4 Hz, 1H), 7.94 (d, J = 8.4 Hz, 1H), 7.73-7.60 (m, 1H), 7.25 (s, 1H), 7.13 (s, 2H), 4.79 (d, J = 3.6 Hz, 1H), 4.06-3.96 (m, 3H), 1.31 (s, 9H), 1.22 (d, J = 5.6 Hz, 3H). |
| 213 | 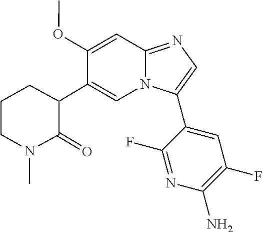 [SMILES: COc1cc2ncc(-c3cc(F)c(N)nc3F)n2cc1C1CCCN(C)C1=O] | 3-(3-(6-amino-2,5- difluoropyridin-3-yl)- 7- methoxyimidazo[1,2- a]pyridin-6-yl)-1- methylpiperidin-2-one | [M + H]⁺ = 388.1 ¹H NMR 400 MHz, DMSO-d₆) δ ppm: 7.89 (d, J = 1.6 Hz, 1H), 7.68 (dd, J = 7.6, 10.4 Hz, 1H), 7.43 (s, 1H), 6.98 (s, 1H), 6.91 (s, 2H), 3.81 (s, 3H), 3.68- 3.58 (m, 1H), 3.41-3.35 (m, 2H), 2.84 (s, 3H), 2.04-1.74 (m, 4H). |
| 214 | 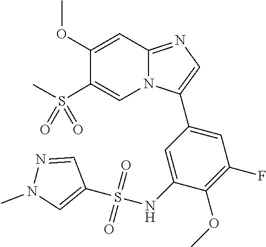 [SMILES: COc1cc2ncc(-c3cc(F)c(OC)c(NS(=O)(=O)c4cnn(C)c4)c3)n2cc1S(C)(=O)=O] | N-(3-fluoro-2- methoxy-5-(7- methoxy-6- (methylsulfonyl)imida zo[1,2-a]pyridin-3- yl)phenyl)-1-methyl- 1H-pyrazole-4- sulfonamide | [M + H] ⁺ = 510.1 ¹H NMR: (400 MHz, DMSO-d₆) δ ppm: 8.69 (s, 1H), 8.28 (s, 1H), 8.14 (s, 1H), 7.80-7.68 (m, 2H), 7.47-7.38 (m, 2H), 7.31 (s, 1H), 4.02 (s, 3H), 3.84 (s, 3H), 3.74 (d, J = 0.8 Hz, 3H), 3.33 (s, 3H). |
| 215 | 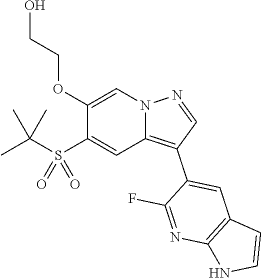 [SMILES: CC(C)(C)S(=O)(=O)c1cc2c(-c3cc4cc[nH]c4nc3F)cnn2cc1OCCO] | 2-((5-(tert- butylsulfonyl)-3-(6- fluoro-1H- pyrrolo[2,3-b]pyridin- 5-yl)pyrazolo[1,5- a]pyridin-6- yl)oxy)ethan-1-ol | [M + H] ⁺ = 433.0 ¹H NMR: (400 MHz, DMSO-d₆) δ ppm: 11.91 (br s, 1H), 8.80 (s, 1H), 8.36 (s, 1H), 8.29 (d, J = 9.6 Hz, 1H), 8.14 (d, J = 1.6 Hz, 1H), 7.57-7.42 (m, 1H), 6.57 (dd, J = 1.6, 3.6 Hz, 1H), 4.83 (t, J = 5.2 Hz, 1H), 4.18 (t, J = 5.2 Hz, 2H), 3.82-3.71 (m, 2H), 1.33 (s, 9H). |
| 216 | 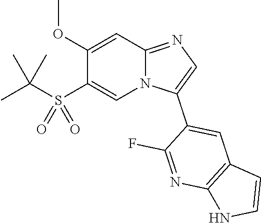 [SMILES: COc1cc2ncc(-c3cc4cc[nH]c4nc3F)n2cc1S(=O)(=O)C(C)(C)C] | 6-(tert-butylsulfonyl)- 3-(6-fluoro-1H- pyrrolo[2,3-b]pyridin- 5-yl)-7- methoxyimidazo[1,2- a]pyridine | [M + H] ⁺ = 403.1 ¹H NMR: (400 MHz, DMSO-d₆) δ ppm: 12.08 (s, 1H), 8.39 (d, J = 2.8 Hz, 1H), 8.31 (d, J = 9.6 Hz, 1H), 7.78 (s, 1H), 7.57 (d, J = 3.6 Hz, 1H), 7.31 (s, 1H), 6.61 (d, J = 3.6 Hz, 1H), 3.95 (s, 3H), 1.29 (s, 9H). |
| 217 | 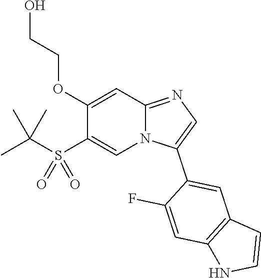 [SMILES: CC(C)(C)S(=O)(=O)c1cn2c(-c3cc4cc[nH]c4cc3F)cnc2cc1OCCO] | 2-((6-(tert- butylsulfonyl)-3-(6- fluoro-1H-indol-5- yl)imidazo[1,2- a]pyridin-7- yl)oxy)ethan-1-ol | [M + H] ⁺ = 432.0 ¹H NMR: (400 MHz, DMSO-d₆) δ ppm: 11.44 (s, 1H), 8.36 (d, J = 2.8 Hz, 1H), 7.74 (d, J = 7.6 Hz, 1H), 7.71 (s, 1H), 7.49-7.42 (m, 2H), 7.30 (s, 1H), 6.54 (s, 1H), 4.82 (t, J = 5.2 Hz, 1H), 4.20 (t, J = 4.8 Hz, 2H), 3.81-3.73 (m, 2H), 1.31 (s, 9H). |
| 218 | 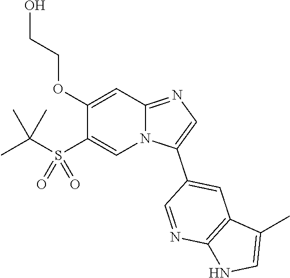 [SMILES: Cc1c[nH]c2ncc(-c3cnc4cc(OCCO)c(S(=O)(=O)C(C)(C)C)cn34)cc12] | 2-((6-(tert- butylsulfonyl)-3-(3- methyl-1H- pyrrolo[2,3-b]pyridin- 5-yl)imidazo[1,2- a]pyridin-7- yl)oxy)ethan-1-ol | [M + H] ⁺ = 429.1 ¹H NMR: (400 MHz, DMSO-d₆) δ ppm: 11.60 (s, 1H), 8.64 (s, 1H), 8.39 (d, J = 1.2 Hz, 1H), 8.14 (s, 1H), 7.74 (s, 1H), 7.37 (s, 1H), 7.28 (s, 1H), 4.93- 4.72 (m, 1H), 4.19 (t, J = 4.8 Hz, 2H), 3.83- 3.75 (m, 2H), 2.28 (s, 3H), 1.32 (s, 9H). |
| 219 | 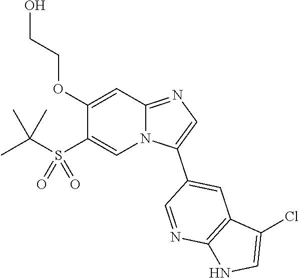 [SMILES: CC(C)(C)S(=O)(=O)c1cn2c(-c3cnc4[nH]cc(Cl)c4c3)cnc2cc1OCCO] | 2-((6-(tert- butylsulfonyl)-3-(3- chloro-1H- pyrrolo[2,3-b]pyridin- 5-yl)imidazo[1,2- a]pyridin-7- yl)oxy)ethan-1-ol | [M + H] ⁺ = 449.0). ¹H NMR: (400 MHz, DMSO-d₆) δ ppm: 12.28 (s, 1H), 8.66 (s, 1H), 8.53 (d, J = 2.0 Hz, 1H), 8.15 (d, J = 2.0 Hz, 1H), 7.85-7.77 (m, 2H), 7.30 (s, 1H), 4.82 (t, J = 4.8 Hz, 1H), 4.20 (t, J = 4.8 Hz, 2H), 3.78 (d, J = 4.8 Hz, 2H), 1.33 (s, 9H). |
| 220 | 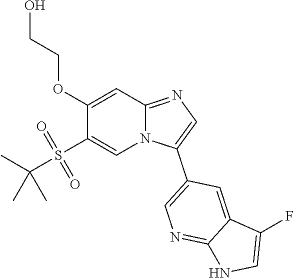 [SMILES: CC(C)(C)S(=O)(=O)c1cn2c(-c3cnc4[nH]cc(F)c4c3)cnc2cc1OCCO] | 2-((6-(tert- butylsulfonyl)-3-(3- fluoro-1H- pyrrolo[2,3-b]pyridin- 5-yl)imidazo[1,2- a]pyridin-7- yl)oxy)ethan-1-ol | [M + H] ⁺ = 433.0 ¹H NMR: (400 MHz, DMSO-d₆) δ ppm: 11.81 (s, 1H), 8.64 (s, 1H), 8.49 (d, J = 2.0 Hz, 1H), 8.43 (s, 0.2H), 8.26 (d, J = 1.6 Hz, 1H), 7.78 (s, 1H), 7.62 (d, J = 1.6 Hz, 1H), 7.30 (s, 1H), 4.86-4.80 (m, 1H), 4.19 (t, J = 4.8 Hz, 2H), 3.78 (d, J = 4.4 Hz, 2H), 1.32 (s, 9H). |
| 221 | 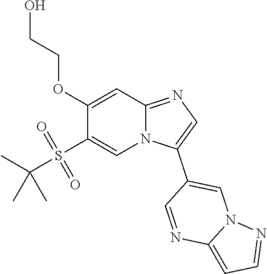 [SMILES: CC(C)(C)S(=O)(=O)c1cn2c(-c3cnc4ccnn4c3)cnc2cc1OCCO] | 2-((6-(tert- butylsulfonyl)-3- (pyrazolo[1,5- a]pyrimidin-6- yl)imidazo[1,2- a]pyridin-7- yl)oxy)ethan-1-ol | [M + H] ⁺ = 416.1 ¹H NMR: (400 MHz, DMSO-d₆) δ ppm: 9.48 (s, 1H), 8.77 (d, J = 1.6 Hz, 1H), 8.74 (s, 1H), 8.44 (s, 0.2H), 8.33 (d, J = 2.0 Hz, 1H), 7.88 (s, 1H), 7.32 (s, 1H), 6.86 (s, 1H), 4.83 (s, 1H), 4.20 (t, J = 4.8 Hz, 2H), 3.79 (s, 2H), 1.33 (s, 9H). |
| 222 | 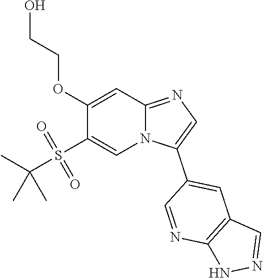 [SMILES: CC(C)(C)S(=O)(=O)c1cn2c(-c3cnc4[nH]ncc4c3)cnc2cc1OCCO] | 2-((6-(tert- butylsulfonyl)-3-(1H- pyrazolo[3,4- b]pyridin-5- yl)imidazo[1,2- a]pyridin-7- yl)oxy)ethan-1-ol | [M + H] ⁺ = 416.0). ¹H NMR: (400 MHz, DMSO-d₆) δ ppm: 14.00-13.77 (m, 1H), 8.74 (d, J = 2.0 Hz, 1H), 8.63 (s, 1H), 8.49 (d, J = 2.0 Hz, 1H), 8.29 (s, 1H), 7.82 (s, 1H), 7.31 (s, 1H), 4.82 (t, J = 5.2 Hz, 1H), 4.20 (t, J = 5.2 Hz, 2H), 3.81-3.75 (m, 2H), 1.32 (s, 9H). |
| 223 | 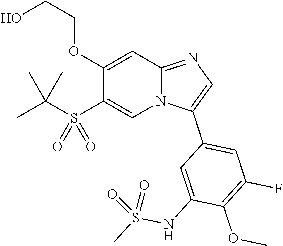 [SMILES: COc1c(F)cc(-c2cnc3cc(OCCO)c(S(=O)(=O)C(C)(C)C)cn23)cc1NS(C)(=O)=O] | N-(5-(6-(tert- butylsulfonyl)-7-(2- hydroxyethoxy)imidazo [1,2-a]pyridin-3- yl)-3-fluoro-2- methoxyphenyl) methanesulfonamide | [M + H] ⁺ = 516.0 ¹H NMR: (400 MHz, DMSO-d₆) δ ppm: 9.57 (s, 1H), 8.72 (s, 1H), 7.77 (s, 1H), 7.45-7.36 (m, 2H), 7.29 (s, 1H), 4.81 (t, J = 5.2 Hz, 1H), 4.18 (t, J = 4.8 Hz, 2H), 3.94 (s, 3H), 3.77 (t, J = 4.8 Hz, 2H), 3.11 (s, 3H), 1.34 (s, 9H). |
| 224 | 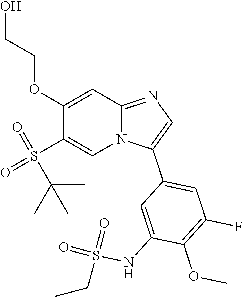 [SMILES: CCS(=O)(=O)Nc1cc(-c2cnc3cc(OCCO)c(S(=O)(=O)C(C)(C)C)cn23)cc(F)c1OC] | N-(5-(6-(tert- butylsulfonyl)-7-(2- hydroxyethoxy)imidazo [1,2-a]pyridin-3- yl)-3-fluoro-2- methoxyphenyl) ethanesulfonamide | [M + H] ⁺ = 530.0 ¹H NMR: (400 MHz, DMSO-d₆) δ ppm: 9.63-9.50 (m, 1H), 8.71 (s, 1H), 7.76 (s, 1H), 7.42 (s, 1H), 7.40-7.35 (m, 1H), 7.29 (s, 1H), 4.81 (t, J = 5.2 Hz, 1H), 4.18 (t, J = 5.2 Hz, 2H), 3.94 (d, J = 1.6 Hz, 3H), 3.80-3.75 (m, 2H), 3.23-3.17 (m, 2H), 1.35 (s, 9H), 1.27 (t, J = 7.2 Hz, 3H). |
| 225 | 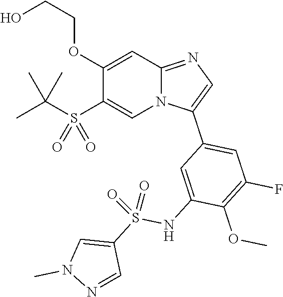 [SMILES: COc1c(F)cc(-c2cnc3cc(OCCO)c(S(=O)(=O)C(C)(C)C)cn23)cc1NS(=O)(=O)c1cnn(C)c1] | N-(5-(6-(tert- butylsulfonyl)-7-(2- hydroxyethoxy) imidazo[1,2-a]pyridin-3- yl)-3-fluoro-2- methoxyphenyl)-1- methyl-1H-pyrazole- 4-sulfonamide | [M + H] ⁺ = 582.0 ¹H NMR: (400 MHz, DMSO-d₆) δ ppm: 9.94 (s, 1H), 8.71 (s, 1H), 8.27 (s, 1H), 7.77 (s, 1H), 7.74 (s, 1H), 7.44 (s, 1H), 7.34 (dd, J = 1.6, 11.2 Hz, 1H), 7.30 (s, 1H), 4.81 (t, J = 5.2 Hz, 1H), 4.19 (t, J = 4.0 Hz, 2H), 3.83 (s, 3H), 3.81-3.75 (m, 2H), 3.73 (s, 3H), 1.36 (s, 9H). |
| 226 | 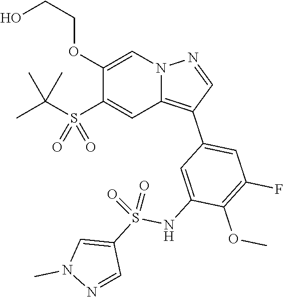 [SMILES: COc1c(F)cc(-c2cnn3cc(OCCO)c(S(=O)(=O)C(C)(C)C)cc23)cc1NS(=O)(=O)c1cnn(C)c1] | N-(5-(5-(tert- butylsulfonyl)-6-(2- hydroxyethoxy)pyrazolo [1,5-a]pyridin-3- yl)-3-fluoro-2- methoxyphenyl)-1- methyl-1H-pyrazole- 4-sulfonamide | [M + H] ⁺ = 582.2 ¹H NMR: (400 MHz, DMSO-d₆) δ ppm: 9.87 (s, 1H), 8.79 (s, 1H), 8.41 (s, 1H), 8.32-8.22 (d, J = 8.0 Hz, 2H), 7.78 (s, 1H), 7.51 (s, 1H), 7.39-7.29 (d, J = 12.4 Hz, 1H), 4.86-4.76 (t, J = 5.2 Hz, 1H), 4.22-4.13 (t, J = 4.8 Hz, 2H), 3.82 (s, 3H), 3.80-3.77 (m, 2H), 3.72 (s, 3H), 1.37 (s, 9H). |
| Int. 151 | 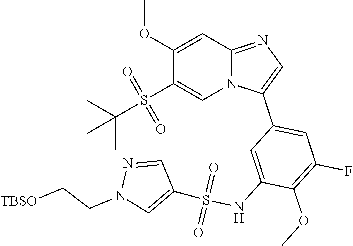 [SMILES: COc1cc2ncc(-c3cc(F)c(OC)c(NS(=O)(=O)c4cnn(CCO[Si](C)(C)C(C)(C)C)c4)c3)n2cc1S(=O)(=O)C(C)(C)C] | 1-(2-((tert- butyldimethylsilyl) oxy)ethyl)-N-(5-(6-(tert- butylsulfonyl)-7- methoxyimidazo[1,2- a]pyridin-3-yl)-3- fluoro-2- methoxyphenyl)-1H- pyrazole-4- sulfonamide | [M + H] ⁺ = 696.4 |
| 227 | 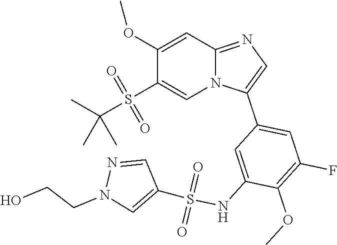 [SMILES: COc1cc2ncc(-c3cc(F)c(OC)c(NS(=O)(=O)c4cnn(CCO)c4)c3)n2cc1S(=O)(=O)C(C)(C)C] | N-(5-(6-(tert- butylsulfonyl)-7- methoxyimidazo[1,2- a]pyridin-3-yl)-3- fluoro-2- methoxyphenyl)-1-(2- hydroxyethyl)-1H- pyrazole-4- sulfonamide | [M + H] ⁺ = 582.0 ¹H NMR: (400 MHz, DMSO-d₆) δ ppm: 9.92 (s, 1H), 8.71 (s, 1H), 8.25 (s, 1H), 7.79- 7.73(m, 2H), 7.44 (s, 1H), 7.40-7.31 (m, 1H), 7.28 (s, 1H), 4.89 (t, J = 4.8 Hz, 1H), 4.15 (t, J = 5.2 Hz, 2H), 3.94 (s, 3H), 3.71 (d, J = 1.2 Hz, 3H), 3.70-3.65 (m, 2H), 1.34 (s, 9H). |
| Int. 152 | 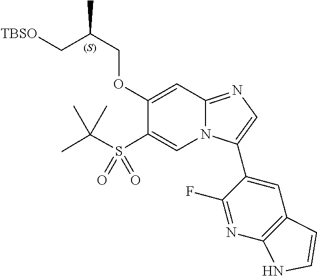 | (S)-7-(3-((tert- butyldimethylsilyl) oxy)-2-methylpropoxy)- 6-(tert-butylsulfonyl)- 3-(6-fluoro-1H- pyrrolo[2,3-b]pyridin- 5-yl)imidazo[1,2- a]pyridine | [M + H] ⁺ = 575.3 ¹H NMR: (400 MHz, DMSO-d₆) δ ppm: 12.07 (s, 1H), 8.38 (s, 1H), 8.31 (d, J = 9.6 Hz, 1H), 7.77 (s, 1H), 7.66-7.50 (m, 1H), 7.27 (s, 1H), 6.61 (dd, J = 1.6, 3.2 Hz, 1H), 4.17- 4.00 (m, 2H), 3.79-3.60 (m, 2H), 2.14-2.04 (m, 1H), 1.30 (s, 9H), 1.03 (d, J = 6.8 Hz, 3H), 0.87 (s, 9H), 0.03 (s, 6H). |
| 228 | 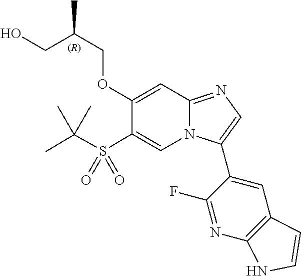 | (R)-3-((6-(tert- butylsulfonyl)-3-(6- fluoro-1H- pyrrolo[2,3-b]pyridin- 5-yl)imidazo[1,2- a]pyridin-7-yl)oxy)-2- methylpropan-1-ol | [M + H] ⁺ = 461.1 ¹H NMR: (400 MHz, DMSO-d₆) δ ppm: 12.07 (s, 1H), 8.38 (s, 1H), 8.31 (d, J = 9.2 Hz, 1H), 7.77 (s, 1H), 7.57 (s, 1H), 7.25 (s, 1H), 6.61 (d, J = 2.8 Hz, 1H), 4.58 (t, J = 5.2 Hz, 1H), 4.19-4.00 (m, 2H), 3.52-3.41 (m, 2H), 2.17-1.93 (m, 1H), 1.30 (s, 9H), 1.01 (d, J = 6.8 Hz, 3H). |
| 229 | 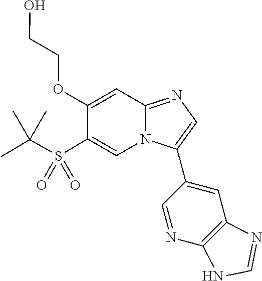 [SMILES: CC(C)(C)S(=O)(=O)c1cn2c(-c3cnc4[nH]cnc4c3)cnc2cc1OCCO] | 2-((6-(tert- butylsulfonyl)-3-(3H- imidazo[4,5- b]pyridin-6- yl)imidazo[1,2- a]pyridin-7- yl)oxy)ethan-1-ol | [M + H] ⁺ = 416.0 ¹H NMR: (400 MHz, DMSO-d₆) δ ppm: 8.66 (s, 1H), 8.56 (d, J = 8.4 Hz, 2H), 8.28 (s, 1H), 7.81 (s, 1H), 7.30 (s, 1H), 4.87-4.76 (m, 1H), 4.24-4.16 (m, 2H), 3.85-3.76 (m, 2H), 1.32 (s, 9H). |
| 230 | 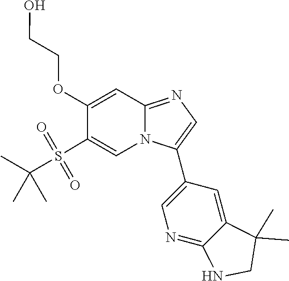 [SMILES: CC1(C)CNc2ncc(-c3cnc4cc(OCCO)c(S(=O)(=O)C(C)(C)C)cn34)cc21] | 2-((6-(tert- butylsulfonyl)-3-(3,3- dimethyl-2,3-dihydro- 1H-pyrrolo[2,3- b]pyridin-5- yl)imidazo[1,2- a]pyridin-7- yl)oxy)ethan-1-ol | [M + H] ⁺ = 445.1 ¹H NMR: (400 MHz, DMSO-d₆) δ ppm: 8.51 (s, 1H), 8.43-8.37 (m, 0.3H), 7.91 (d, J = 1.6 Hz, 1H), 7.59 (s, 1H), 7.47 (d, J = 2.0 Hz, 1H), 7.23 (s, 1H), 6.83 (s, 1H), 4.86-4.77 (m, 1H), 4.17 (t, J = 4.8 Hz, 2H), 3.77 (s, 2H), 3.40- 3.38 (m, 2H), 1.33 (s, 9H), 1.28 (s, 6H). |
| 231 | 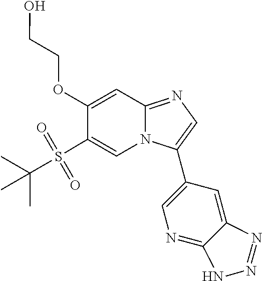 [SMILES: CC(C)(C)S(=O)(=O)c1cn2c(-c3cnc4[nH]nnc4c3)cnc2cc1OCCO] | 2-((3-(3H- [1,2,3]triazolo[4,5- b]pyridin-6-yl)-6- (tert- butylsulfonyl)imidazo [1,2-a]pyridin-7- yl)oxy)ethan-1-ol | [M + H] ⁺ = 417.0. ¹H NMR: (400 MHz, DMSO-d₆) δ ppm: 8.83 (s, 1H), 8.74 (s, 1H), 8.59 (s, 1H), 8.16 (s, 0.23H), 7.90 (s, 1H), 7.33 (s, 1H), 4.88-4.80 (m, 1H), 4.20 (t, J = 5.2 Hz, 2H), 3.85-3.74 (m, 2H), 1.33 (s, 9H). |
| 232 | 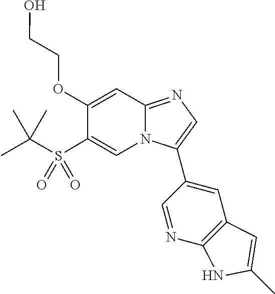 [SMILES: Cc1cc2cc(-c3cnc4cc(OCCO)c(S(=O)(=O)C(C)(C)C)cn34)cnc2[nH]1] | 2-((6-(tert- butylsulfonyl)-3-(2- methyl-1H- pyrrolo[2,3-b]pyridin- 5-yl)imidazo[1,2- a]pyridin-7- yl)oxy)ethan-1-ol | [M + H] ⁺ = 429.0 ¹H NMR: (400 MHz, DMSO-d₆) δ ppm: 11.75 (s, 1H), 8.62 (s, 1H), 8.28 (d, J = 1.6 Hz, 1H), 8.01 (d, J = 2.0 Hz, 1H), 7.71 (s, 1H), 7.28 (s, 1H), 6.26 (s, 1H), 4.80 (t, J = 5.2 Hz, 1H), 4.19 (t, J = 4.8 Hz, 2H), 3.80-3.76 (m, 2H), 2.44 (s, 3H), 1.31 (s, 9H). |
| 233 | 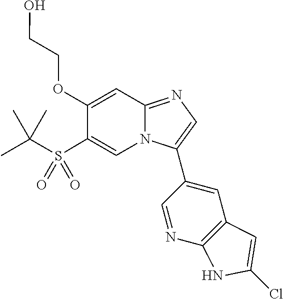 [SMILES: CC(C)(C)S(=O)(=O)c1cn2c(-c3cnc4[nH]c(Cl)cc4c3)cnc2cc1OCCO] | 2-((6-(tert- butylsulfonyl)-3-(2- chloro-1H- pyrrolo[2,3-b]pyridin- 5-yl)imidazo[1,2- a]pyridin-7- yl)oxy)ethan-1-ol | [M + H] ⁺ = 449.0 ¹H NMR: (400 MHz, DMSO-d₆) δ ppm: 8.62 (s, 1H), 8.41 (d, J = 2.0 Hz, 1H), 8.12 (d, J = 2.0 Hz, 1H), 7.75 (s, 1H), 7.29 (s, 1H), 6.61 (s, 1H), 4.85-4.82 (m, 1H), 4.19 (t, J = 4.8 Hz, 2H), 3.82-3.74 (m, 2H), 1.31 (s, 9H). |
| 234 | 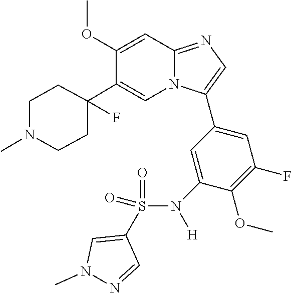 [SMILES: COc1cc2ncc(-c3cc(F)c(OC)c(NS(=O)(=O)c4cnn(C)c4)c3)n2cc1C1(F)CCN(C)CC1] | N-(3-fluoro-5-(6-(4- fluoro-1- methylpiperidin-4-yl)- 7- methoxyimidazo[1,2- a]pyridin-3-yl)-2- methoxyphenyl)-1- methyl-1H-pyrazole- 4-sulfonamide | [M + H] ⁺ = 547.2 ¹H NMR: (400 MHz, DMSO-d₆) δ ppm: 8.28 (s, 1H), 8.22 (s, 1H), 7.72 (m, 1H), 7.58 (s, 1H), 7.37-7.30 (m, 2H), 7.14 (d, J = 2.0 Hz, 1H), 3.92 (s, 3H), 3.84 (s, 3H), 3.73-3.70 (m, 3H), 2.76-2.68 (m, 2H), 2.62-2.54 (m, 1H), 2.49-2.43 (m, 1H), 2.31-2.24 (m, 2H), 2.24 (s, 3H), 1.84 ( t, J = 12.4 Hz, 2H). |
| 235 | 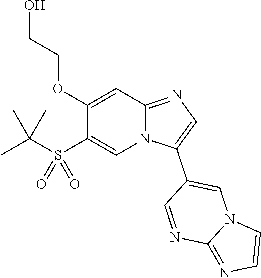 [SMILES: CC(C)(C)S(=O)(=O)c1cn2c(-c3cnc4nccn4c3)cnc2cc1OCCO] | 2-((6-(tert- butylsulfonyl)-3- (imidazo[1,2- a]pyrimidin-6- yl)imidazo[1,2- a]pyridin-7- yl)oxy)ethan-1-ol | [M + H] ⁺ = 416.0 ¹H NMR: (400 MHz, DMSO-d₆) δ ppm: 9.26 (d, J = 2.4 Hz, 1H), 8.75 (d, J = 2.4 Hz, 1H), 8.72 (s, 1H), 8.03 (s, 1H), 7.84-7.80 (m, 2H), 7.32 (s, 1H), 4.82 (t, J = 5.2 Hz, 1H), 4.20 (t, J = 4.8 Hz, 2H), 3.81-3.76 (m, 2H), 1.32 (s, 9H). |
| 236 | 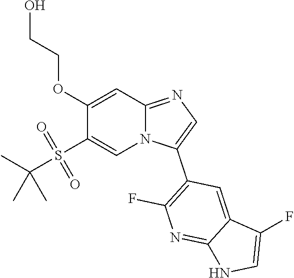 [SMILES: CC(C)(C)S(=O)(=O)c1cn2c(-c3cc4c(F)c[nH]c4nc3F)cnc2cc1OCCO] | 2-((6-(tert- butylsulfonyl)-3-(3,6- difluoro-1H- pyrrolo[2,3-b]pyridin- 5-yl)imidazo[1,2- a]pyridin-7- yl)oxy)ethan-1-ol | [M + H] ⁺ = 451.0 ¹H NMR: (400 MHz, DMSO-d₆) δ ppm: 11.93 (s, 1H), 8.45- 8.36 (m, 2H), 7.80 (s, 1H), 7.57 (d, J = 2.0 Hz, 1H), 7.33 (s, 1H), 4.82 (t, J = 5.2 Hz, 1H), 4.20 (t, J = 4.8 Hz, 2H), 3.81-3.75 (m, 2H), 1.31 (s, 9H) |
| 237 | 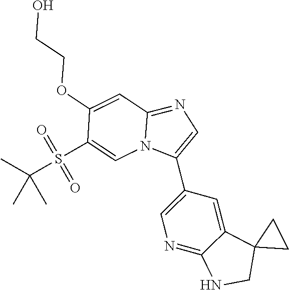 [SMILES: CC(C)(C)S(=O)(=O)c1cn2c(-c3cnc4c(c3)C3(CC3)CN4)cnc2cc1OCCO] | 2-((6-(tert- butylsulfonyl)-3- (1′,2′- dihydrospiro [cyclopropane- 1,3′-pyrrolo[ 2,3- b]pyridin] -5′- yl)imidazo[1,2- a]pyridin-7- yl)oxy)ethan-1-ol | [M + H] ⁺ = 443.0 ¹H NMR: (400 MHz, DMSO-d₆) δ ppm: 8.48 (s,1H), 8.34 (s, 0.51H), 7.83 (d, J = 2.0 Hz, 1H), 7.56 (s, 1H), 7.21 (s, 1H), 7.04 (d, J = 2.0 Hz, 1H), 6.98 (s, 1H), 4.84-4.77 (m, 1H), 4.16 (t, J = 4.8 Hz, 2H), 3.76 (t, J = 4.8 Hz, 2H), 3.59 (s, 2H), 1.32 (s, 9H), 0.88-1.21 (m, 4H). |
| 238 | 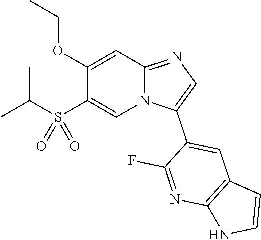 [SMILES: CCOc1cc2ncc(-c3cc4cc[nH]c4nc3F)n2cc1S(=O)(=O)C(C)C] | 7-ethoxy-3-(6-fluoro- 1H-pyrrolo[ 2,3- b]pyridin-5-yl)-6- (isopropylsulfonyl) imidazo[1,2-a]pyridine | [M + H] ⁺ = 403.0 ¹H NMR: (400 MHz, DMSO-d₆) δ ppm: 12.08 ( s, 1H), 8.40 (d, J = 2.4 Hz, 1H), 8.33 (d, J = 9.6 Hz, 1H), 7.76 (s, 1H), 7.57 (d, J = 3.6 Hz, 1H), 7.32 (s, 1H), 6.62 (d, J = 3.2 Hz, 1H), 4.33-4.25 (m, 2H), 3.78-3.65 (m, 1H), 1.41 (t, J = 7.2 Hz, 3H), 1.20 (d, J = 6.8 Hz, 6H). |
| 239 | 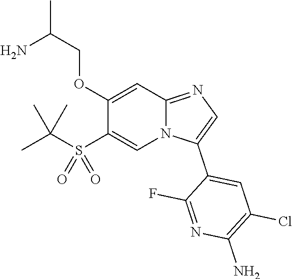 [SMILES: CC(N)COc1cc2ncc(-c3cc(Cl)c(N)nc3F)n2cc1S(=O)(=O)C(C)(C)C] | 5-(7-(2- aminopropoxy)-6- (tert- butylsulfonyl)imidazo [1,2-a]pyridin-3-yl)- 3-chloro-6- fluoropyridin-2-amine | [M + H] ⁺ = 456.0 ¹H NMR: (400 MHz, DMSO-d₆) δ ppm: 8.37 (d, J = 2.8 Hz, 1H), 7.93 (d, J = 8.4 Hz, 1H), 7.68 (s, 1H), 7.22 (s, 1H), 7.11 (s, 2H), 4.04-3.98 (m, 1H), 3.85-3.80 (m, 1H), 3.23-3.16 (m, 1H), 2.21-1.64 (m, 2H), 1.32 (s, 9H), 1.09 (d, J = 6.4 Hz, 3H). |
| 240 | 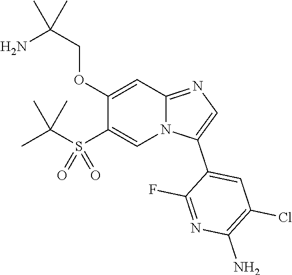 [SMILES: CC(C)(N)COc1cc2ncc(-c3cc(Cl)c(N)nc3F)n2cc1S(=O)(=O)C(C)(C)C] | 1-((3-(6-amino-5- chloro-2- fluoropyridin-3-yl)-6- (tert- butylsulfonyl)imidazo [1,2-a]pyridin-7- yl)oxy)-2- methylpropan-2-ol | [M + H] ⁺ = 471.0). ¹H NMR: (400 MHz, DMSO-d₆) δ ppm: 8.36 (s, 1H), 7.93 (d, J=8.4 Hz, 1H), 7.68 (s, 1H), 7.24 (s, 1H), 7.12 (s, 2H), 4.53 (s, 1H), 3.90 (s, 2H), 1.32 (s, 9H), 1.26 (s, 6H). |
| 241 | 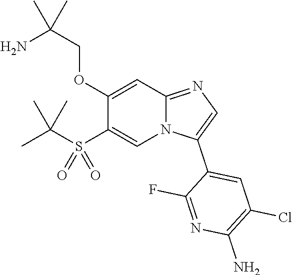 [SMILES: CC(C)(N)COc1cc2ncc(-c3cc(Cl)c(N)nc3F)n2cc1S(=O)(=O)C(C)(C)C] | 5-(7-(2-amino-2- methylpropoxy)-6- (tert- butylsulfonyl)imidazo [1,2-a]pyridin-3-yl)- 3-chloro-6- fluoropyridin-2-amine | [M + H] ⁺ = 470.0 ¹H NMR: (400 MHz, DMSO-d₆) δ ppm: 8.37 (d, J = 2.0 Hz, 1H), 8.24 (s, 0.74H), 7.93 (d, J = 8.4 Hz, 1H), 7.69 (s, 1H), 7.25 (s, 1H), 7.12 (br s, 2H), 3.93 (br s, 2H), 3.41- 3.36 (m, 2H), 1.32 (s, 9H), 1.18 (s, 6H). |
| 242 | 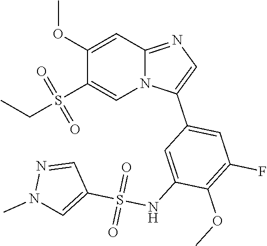 [SMILES: CCS(=O)(=O)c1cn2c(-c3cc(F)c(OC)c(NS(=O)(=O)c4cnn(C)c4)c3)cnc2cc1OC] | N-(5-(6- (ethylsulfonyl)-7- methoxyimidazo[1,2- a]pyridin-3-yl)-3- fluoro-2- methoxyphenyl)-1- methyl-1H-pyrazole- 4-sulfonamide | [M + H] ⁺ = 524.0 ¹H NMR: (400 MHz, DMSO-d₆) δ ppm: 8.68 (s, 1H), 8.26 (s, 1H), 8.16 (s, 0.25H), 7.76 (s, 1H), 7.71 (s, 1H), 7.43- 7.37 (m, 2H), 7.31 (s, 1H), 4.00 (s, 3H), 3.84 (s, 3H), 3.74 (d, J = 1.6 Hz, 3H), 3.49-3.41 (m, 2H), 1.17 (t, J = 7.4 Hz, 3H). |
| Int. 153 | 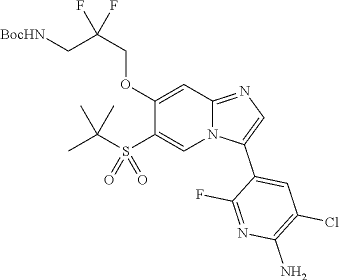 [SMILES: CC(C)(C)OC(=O)NCC(F)(F)COc1cc2ncc(-c3cc(Cl)c(N)nc3F)n2cc1S(=O)(=O)C(C)(C)C] | tert-butyl (3-((3-(6- amino-5-chloro-2- fluoropyridin-3-yl)-6- (tert- butylsulfonyl)imidazo [1,2-a]pyridin-7- yl)oxy)-2,2- difluoropropyl) carbamate | ¹H NMR: (400 MHz, DMSO-d₆) δ ppm: 8.39 (d, J = 2.4 Hz, 1H), 7.94 (d, J = 8.4 Hz, 1H), 7.72 (s, 1H), 7.35 (s, 1H), 7.32-7.27 (m, 1H), 7.14 (s, 2H), 4.53- 4.44 (m, 2H), 3.73-3.62 (m, 2H), 1.39 (s, 9H), 1.30 (s, 9H). |
| 243 | 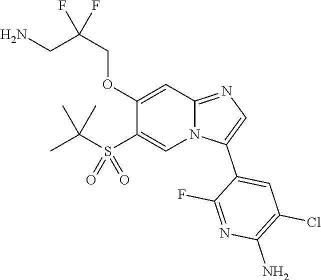 [SMILES: CC(C)(C)S(=O)(=O)c1cn2c(-c3cc(Cl)c(N)nc3F)cnc2cc1OCC(F)(F)CN] | 5-(7-(3-amino-2,2- difluoropropoxy)-6- (tert- butylsulfonyl)imidazo [1,2-a]pyridin-3-yl)- 3-chloro-6- fluoropyridin-2-amine | [M + H] ⁺ = 492.0 ¹H NMR: (400 MHz, DMSO-d₆) δ ppm: 8.40 (d, J = 2.4 Hz, 1H), 7.94 (d, J = 8.4 Hz, 1H), 7.73 (s, 1H), 7.41 (s, 1H), 7.14 (s, 2H), 4.57 (t, J = 12.4 Hz, 2H), 3.29-3.22 (m, 2H), 1.30 (s, 9H). |
| 244 | 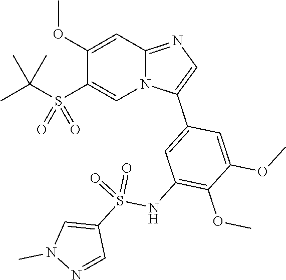 [SMILES: COc1cc2ncc(-c3cc(NS(=O)(=O)c4cnn(C)c4)c(OC)c(OC)c3)n2cc1S(=O)(=O)C(C)(C)C] | N-(5-(6-(tert- butylsulfonyl)-7- methoxyimidazo[1,2- a]pyridin-3-yl)-2,3- dimethoxyphenyl)-1- methyl-1H-pyrazole- 4-sulfonamide | [M + H]⁺ = 564.1 ¹H NMR: (400 MHz, DMSO-d₆) δ ppm: 9.58 (s, 1H), 8.75 (s, 1H), 8.25 (s, 1H), 7.74 (d, J = 8.4 Hz, 2H), 7.32- 7.20 (m, 2H), 7.06 (s, 1H), 3.94 (s, 3H), 3.86- 3.80 (m, 6H), 3.61 (s, 3H), 1.34 (s, 9H). |
| 245 | 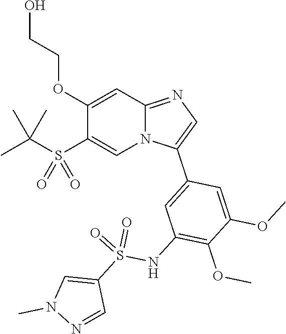 [SMILES: COc1cc(-c2cnc3cc(OCCO)c(S(=O)(=O)C(C)(C)C)cn23)cc(NS(=O)(=O)c2cnn(C)c2)c1OC] | N-(5-(6-(tert- butylsulfonyl)-7-(2- hydroxyethoxy)imidazo [1,2-a]pyridin-3- yl)-2,3- dimethoxyphenyl)-1- methyl-1H-pyrazole- 4-sulfonamide | [M + H] ⁺ = 594.1 ¹H NMR: (400 MHz, DMSO-d₆) δ ppm: 9.81-9.35 (m, 1H), 8.76 (s, 1H), 8.24 (s, 1H), 7.73 (d, J = 9.6 Hz, 2H), 7.28 (s, 1H), 7.24 (s, 1H), 7.04 (s, 1H), 4.81 (t, J = 4.8 Hz, 1H), 4.19 (t, J = 4.8 Hz, 2H), 3.86-3.78 (m, 6H), 3.80-3.75 (m, 2H), 3.61 (s, 3H), 1.36 (s, 9H). |
| 246 | 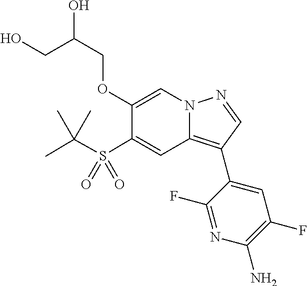 [SMILES: CC(C)(C)S(=O)(=O)c1cc2c(-c3cc(F)c(N)nc3F)cnn2cc1OCC(O)CO] | 3-((3-(6-amino-2,5- difluoropyridin-3-yl)- 5-(tert- butylsulfonyl)pyrazolo [1,5-a]pyridin-6- yl)oxy)propane-1,2- diol | [M + H] ⁺ = 457.0 ¹H NMR: (400 MHz, DMSO-d₆) δ ppm: 8.72 (s, 1H), 8.27 (s, 1H), 8.11 (s, 1H), 7.86-7.76 (m, 1H), 6.80 (s, 2H), 4.94-4.83 (m, 1H), 4.66 (t, J = 5.6 Hz, 1H), 4.15-4.10 (m, 1H), 4.09-4.01 (m, 1H), 3.89-3.80 (m, 1H), 3.56-3.48 (m, 2H), 1.32 (s, 9H). |
| 247 | 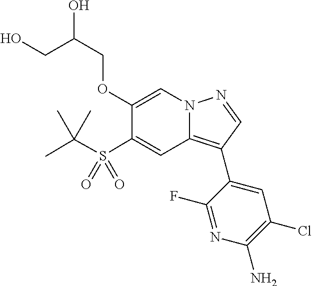 [SMILES: CC(C)(C)S(=O)(=O)c1cc2c(-c3cc(Cl)c(N)nc3F)cnn2cc1OCC(O)CO] | 3-((3-(6-amino-5- chloro-2- fluoropyridin-3-yl)-5- (tert- butylsulfonyl)pyrazolo [1,5-a]pyridin-6- yl)oxy)propane-1,2- diol | [M + H] ⁺ = 473.0 ¹H NMR: (400 MHz, DMSO-d₆) δ ppm: 8.71 (s, 1H), 8.29 (s, 1H), 8.09 (s, 1H), 7.97 (d, J = 8.6 Hz, 1H), 6.88 (s, 2H), 4.90 (d, J = 4.8 Hz, 1H), 4.66 (t, J = 5.6 Hz, 1H), 4.15-4.10 (m, 1H), 4.08-4.02 (m, 1H), 3.88-3.82 (m, 1H), 3.54-3.49 (m, 2H), 1.32 (s, 9H). |
| 248 | 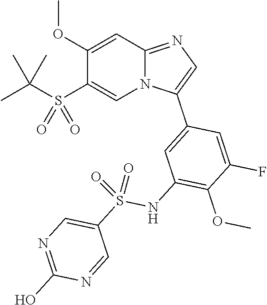 [SMILES: COc1cc2ncc(-c3cc(F)c(OC)c(NS(=O)(=O)c4cnc(O)nc4)c3)n2cc1S(=O)(=O)C(C)(C)C] | N-(5-(6-(tert- butylsulfonyl)-7- methoxyimidazo[1,2- a]pyridin-3-yl)-3- fluoro-2- methoxyphenyl)-2- hydroxypyrimidine-5- sulfonamide | [M + H] ⁺ = 566.0 ¹H NMR: (400 MHz, DMSO-d₆) δ ppm: 9.03 (s, 1H), 8.89 (s, 1H), 8.64 (s, 2H), 8.26 (s, 1H), 8.16 (d, J = 6.4 Hz, 1H), 7.44 (s, 1H), 7.35-7.30 (m, 1H), 4.06 (s, 3H), 3.98 (d, J = 1.2 Hz, 3H), 1.31 (s, 9H). |
| 249 | 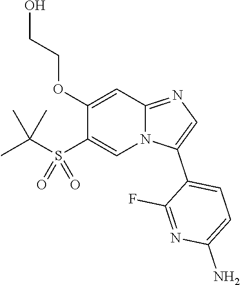 [SMILES: CC(C)(C)S(=O)(=O)c1cn2c(-c3ccc(N)nc3F)cnc2cc1OCCO] | 2-((3-(6-amino-2- fluoropyridin-3-yl)-6- (tert- butylsulfonyl)imidazo [1,2-a]pyridin-7- yl)oxy)ethan-1-ol | [M + H] ⁺ = 409.0 ¹H NMR: (400 MHz, DMSO-d₆) δ ppm: 8.38 (s, 0.4H), 8.30 (d, J = 2.4 Hz, 1H), 7.67 (dd, J = 8.0, 10.0 Hz, 1H), 7.62 (s, 1H), 7.27 (s, 1H), 6.79 (s, 2H), 6.49 (dd, J = 2.0, 8.0 Hz, 1H), 4.81 (s, 1H), 4.18 (t, J = 5.2 Hz, 2H), 3.77 (t, J = 4.8 Hz, 2H), 1.31 (s, 9H). |
| 250 | 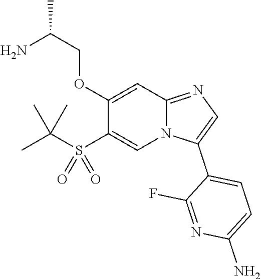 [SMILES: C[C@@H](N)COc1cc2ncc(-c3ccc(N)nc3F)n2cc1S(=O)(=O)C(C)(C)C] | (R)-1-((3-(6-amino-2- fluoropyridin-3-yl)-6- (tert- butylsulfonyl)imidazo [1,2-a]pyridin-7- yl)oxy)propan-2-ol | [M + H] ⁺ = 423.1 ¹H NMR: (400 MHz, DMSO-d₆) δ ppm: 8.30 (d, J = 2.4 Hz, 1H), 8.24 (s, 0.15H), 7.72- 7.59 (m, 2H), 7.24 (s, 1H), 6.78 (s, 2H), 6.55- 6.40 (m, 1H), 4.95-4.55 (m, 1H), 4.06-3.94 (m, 3H), 1.31 (s, 9H), 1.22 (d, J = 5.6 Hz, 3H). |
| 251 | 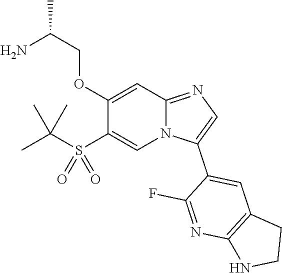 [SMILES: C[C@@H](N)COc1cc2ncc(-c3cc4c(nc3F)NCC4)n2cc1S(=O)(=O)C(C)(C)C] | (R)-1-((6-(tert- butylsulfonyl)-3-(6- fluoro-1H- pyrrolo[2,3-b]pyridin- 5-yl)imidazo[1,2- a]pyridin-7- yl)oxy)propan-2-ol | [M + H] ⁺ = 447.1 ¹H NMR: (400 MHz, DMSO-d₆) δ ppm: 12.08 (s, 1H), 8.39 (d, J = 2.8 Hz, 1H), 8.31 (d, J = 9.6 Hz, 1H), 8.24 (s, 0.1H), 7.77 (s, 1H), 7.69-7.50 (m, 1H), 7.29 (s, 1H), 6.61 (dd, J = 2.0, 3.6 Hz, 1H), 4.80 (s, 1H), 4.16-3.90 (m, 3H), 1.31 (s, 9H), 1.23 (d, J = 5.6 Hz, 3H). |
| 252 | 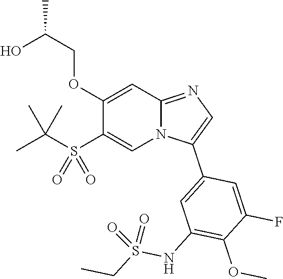 [SMILES: CCS(=O)(=O)Nc1cc(-c2cnc3cc(OC[C@@H](C)O)c(S(=O)(=O)C(C)(C)C)cn23)cc(F)c1OC] | (R)-N-(5-(6-(tert- butylsulfonyl)-7-(2- hydroxypropoxy) imidazo[1,2-a]pyridin-3- yl)-3-fluoro-2- methoxyphenyl) ethanesulfonamide | [M + H] ⁺ = 544.1 ¹H NMR: (400 MHz, DMSO-d₆) δ ppm: 9.57 (s, 1H), 8.70 (s, 1H), 7.76 (s, 1H), 7.42 (s, 1H), 7.40-7.35 (m, 1H), 7.26 (s, 1H), 4.79 (d, J = 4.0 Hz, 1H), 4.06- 3.96 (m, 3H), 3.94 (d, J = 1.6 Hz, 3H), 3.23- 3.17 (m, 2H), 1.35 (s, 9H), 1.27 (t, J = 7.2 Hz, 3H), 1.22 (d, J = 5.6 Hz, 3H). |
| 253 | 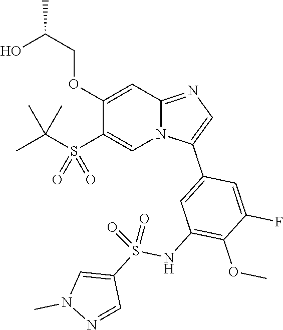 [SMILES: COc1c(F)cc(-c2cnc3cc(OC[C@@H](C)O)c(S(=O)(=O)C(C)(C)C)cn23)cc1NS(=O)(=O)c1cnn(C)c1] | (R)-N-(5-(6-(tert- butylsulfonyl)-7-(2- hydroxypropoxy)imidazo [1,2-a]pyridin-3- yl)-3-fluoro-2- methoxyphenyl)-1- methyl-1H-pyrazole- 4-sulfonamide | [M + H] ⁺ = 596.1 ¹H NMR: (400 MHz, DMSO) δ ppm: 10.72- 9.76 (m, 1H), 8.70 (s, 1H), 8.22 (s, 1H), 8.22 (s, 0.15H), 7.72 (d, J = 8.8 Hz, 2H), 7.42 (s, 1H), 7.31-7.20 (m, 2H), 4.80 (s, 1H), 4.06-3.95 (m, 3H), 3.82 (s, 3H), 3.73 (s, 3H), 1.35 (s, 9H), 1.22 (d, J = 5.6 Hz, 3H). |
| 254 | 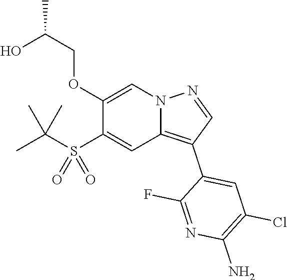 [SMILES: C[C@@H](O)COc1cn2ncc(-c3cc(Cl)c(N)nc3F)c2cc1S(=O)(=O)C(C)(C)C] | (R)-1-((3-(6-amino-5- chloro-2- fluoropyridin-3-yl)-5- (tert- butylsulfonyl)pyrazolo [1,5-a]pyridin-6- yl)oxy)propan-2-ol | [M + H] ⁺ = 457.0 ¹H NMR: (400 MHz, DMSO-d₆) δ ppm: 8.72 (s, 1H), 8.29 (s, 1H), 8.09 (d, J = 1.6 Hz, 1H), 7.97 (d, J = 8.4 Hz, 1H), 6.88 (s, 2H), 4.80 (d, J = 4.0 Hz, 1H), 4.04-3.92 (m, 3H), 1.32 (s, 9H), 1.21 (d, J = 5.6 Hz, 3H). |
| 255 | 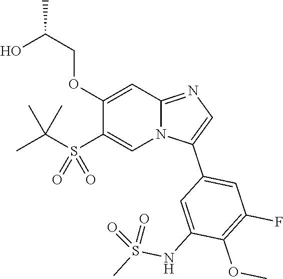 [SMILES: COc1c(F)cc(-c2cnc3cc(OC[C@@H](C)O)c(S(=O)(=O)C(C)(C)C)cn23)cc1NS(C)(=O)=O] | (R)-N-(5-(6-(tert- butylsulfonyl)-7-(2- hydroxypropoxy)imidazo [1,2-a]pyridin-3- yl)-3-fluoro-2- methoxyphenyl) methanesulfonamide | [M + H]⁺ = 530.0 ¹H NMR: (400 MHz, DMSO-d₆) δ ppm: 8.72 (s, 1H), 7.75 (s, 1H), 7.40 (s, 1H), 7.33 (d, J = 11.6 Hz, 1H), 7.26 (s, 1H), 4.79 (d, J = 3.6 Hz, 1H), 4.05-3.96 (m, 3H), 3.93 (s, 3H), 3.09 (s, 3H), 1.34 (s, 9H), 1.22 (d, J = 6.0 Hz, 3H) |
| Int. 154 | 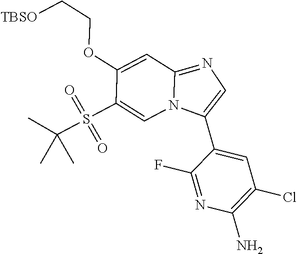 [SMILES: CC(C)(C)[Si](C)(C)OCCOc1cc2ncc(-c3cc(Cl)c(N)nc3F)n2cc1S(=O)(=O)C(C)(C)C] | 3-(6-amino-5-chloro- 2-fluoropyridin-3-yl)- N-(2-((tert- butyldimethylsilyl) oxy)ethyl)-6-(tert- butylsulfonyl)imidazo [1,2-a]pyridin-7- amine | [M + H]⁺ = 556.2 ¹H NMR: (400 MHz, DMSO-d₆) δ ppm: 8.26 (d, J = 2.8 Hz, 1H), 7.65 (d, J = 8.4 Hz, 1H), 7.47 (s, 1H), 6.81- 6.68 (m, 1H), 6.57 (s, 1H), 5.14 (s, 2H), 3.89 (t, J = 5.2 Hz, 2H), 3.30-3.22 (m, 2H), 1.41 (s, 9H), 0.92 (s, 9H), 0.10 (s, 6H) |
| 256 | 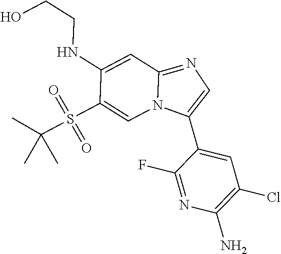 [SMILES: CC(C)(C)S(=O)(=O)c1cn2c(-c3cc(Cl)c(N)nc3F)cnc2cc1NCCO] | 2-((3-(6-amino-5- chloro-2- fluoropyridin-3-yl)-6- (tert- butylsulfonyl)imidazo [1,2-a]pyridin-7- yl)amino)ethan-1-ol | [M + H] ⁺ = 442.0 ¹H NMR: (400 MHz, DMSO-d₆) δ ppm: 8.19 (d, J = 2.0 Hz, 1H), 7.88 (d, J = 8.4 Hz, 1H), 7.52 (s, 1H), 7.06 (br s, 2H), 6.60 (s, 1H), 6.55 (t, J = 4.8 Hz, 1H), 4.92 (t, J = 5.2 Hz, 1H), 3.67-3.61 (m, 2H), 3.24-3.19 (m, 2H), 1.32 (s, 9H). |
| Int. 155 | 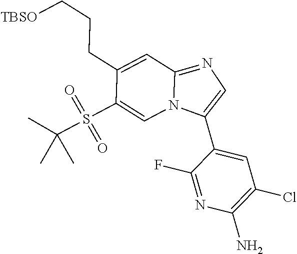 [SMILES: CC(C)(C)[Si](C)(C)OCCCc1cc2ncc(-c3cc(Cl)c(N)nc3F)n2cc1S(=O)(=O)C(C)(C)C] | 5-(7-(3-((tert- butyldimethylsilyl) oxy)propyl)-6-(tert- butylsulfonyl)imidazo [1,2-a]pyridin-3-yl)- 3-chloro-6- fluoropyridin-2-amine | [M + H] ⁺ = 555.1 |
| 257 | 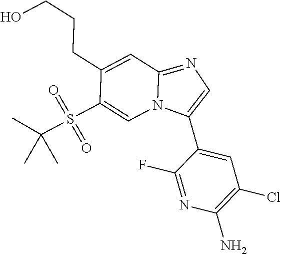 [SMILES: CC(C)(C)S(=O)(=O)c1cn2c(-c3cc(Cl)c(N)nc3F)cnc2cc1CCCO] | 3-(3-(6-amino-5- chloro-2- fluoropyridin-3-yl)-6- (tert- butylsulfonyl)imidazo [1,2-a]pyridin-7- yl)propan-1-ol | [M + H] ⁺ = 441.0 ¹H NMR: (400 MHz, DMSO-d₆) δ ppm: 8.45 (d, J = 2.0 Hz, 1H), 7.95 (d, J = 8.4 Hz, 1H), 7.84 (s, 1H), 7.68 (s, 1H), 7.17 (s, 2H), 4.52 (t, J = 5.2 Hz, 1H), 3.49-3.43 (m, 2H), 3.05-2.96 (m, 2H), 1.85-1.76 (m, 2H), 1.29 (s, 9H). |
| 258 | 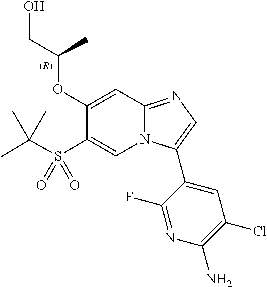 | (R)-2-((3-(6-amino-5- chloro-2- fluoropyridin-3-yl)-6- (tert- butylsulfonyl)imidazo [1,2-a]pyridin-7- yl)oxy)propan-1-ol | [M + H] ⁺ = 457.0 ¹H NMR: (400 MHz, DMSO-d₆) δ ppm: 8.37 (d, J = 2.4 Hz, 1H), 7.93 (d, J = 8.0 Hz, 1H), 7.68 (s, 1H), 7.33 (s, 1H), 7.12 (s, 2H), 4.79-4.76 (m, 1H), 4.75-4.67 (m, 1H), 3.66-3.59 (m, 1H), 3.56-3.49 (m, 1H), 1.32 (s, 9H), 1.29 (d, J = 6.0 Hz, 3H). |
| 259 | 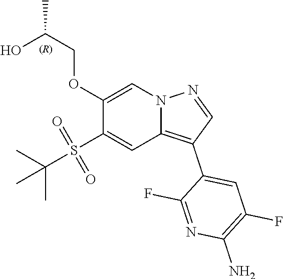 | (R)-1-((3-(6-amino- 2,5-difluoropyridin-3- yl)-5-(tert- butylsulfonyl)pyrazolo [1,5-a]pyridin-6- yl)oxy)propan-2-ol | [M + H] ⁺ = 441.0 ¹H NMR: (400 MHz, DMSO-d₆) δ ppm: 8.72 (s, 1H), 8.27 (s, 1H), 8.10 (d, J = 1.2 Hz, 1H), 7.81 (dd, J = 8.0, 10.8 Hz, 1H), 6.80 (s, 2H), 4.80 (d, J = 3.6 Hz, 1H), 4.05-3.90 (m, 3H), 1.32 (s, 9H), 1.23- 1.18 (m, 3H). |
| Int. 156 | 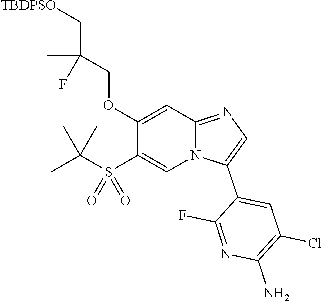 | 5-(7-(3-((tert- butyldiphenylsilyl) oxy)-2-fluoro-2- methylpropoxy)-6- (tert- butylsulfonyl)imidazo [1,2-a]pyridin-3-yl)- 3,6-difluoropyridin-2- amine | [M + H] ⁺ = 711.3 ¹H NMR: (400 MHz, DMSO-d₆) δ ppm: 8.36 (d, J = 2.4 Hz, 1H), 7.76 (dd, J = 7.6, 10.0 Hz, 1H), 7.68 (s, 1H), 7.65-7.63 (m, 4H), 7.46-7.40 (m, 6H), 7.29 (s, 1H), 7.06 (s, 2H), 4.38-4.29 (m, 2H), 4.05-4.03 (m, 1H), 3.95-3.87 (m, 1H), 1.54-1.47 (m, 3H), 1.23 (s, 9H), 1.01 (s, 9H). |
| 260 | 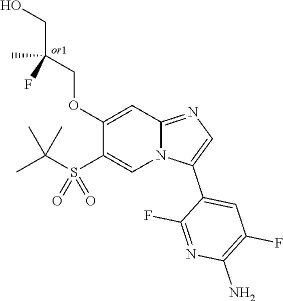 [SMILES: CC(C)(C)S(=O)(=O)c1cn2c(-c3cc(F)c(N)nc3F)cnc2cc1OC[C@@](C)(F)CO] | rel-(R)-3-((3-(6- amino-2,5- difluoropyridin-3-yl)- 6-(tert- butylsulfonyl)imidazo [1,2-a]pyridin-7- yl)oxy)-2-fluoro-2- methylpropan-1-ol | [M + H] ⁺ = 473.0 ¹H NMR: (400 MHz, DMSO-d₆) δ ppm: 8.37 (d, J = 2.0 Hz, 1H), 7.77 (dd, J = 7.2, 10.0 Hz, 1H), 7.68 (s, 1H), 7.26 (s, 1H), 7.06 (s, 2H), 5.09 (t, J = 6.0 Hz, 1H), 4.28-4.21 (m, 2H), 3.64 (dd, J = 6.0, 18.4 Hz, 2H), 3.68-3.61 (m, 3H), 1.30 (s, 9H). |
| 261 | 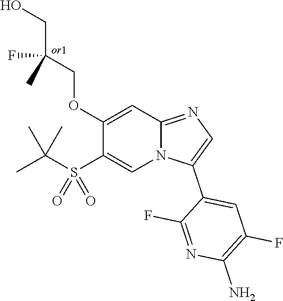 [SMILES: CC(C)(C)S(=O)(=O)c1cn2c(-c3cc(F)c(N)nc3F)cnc2cc1OC[C@](C)(F)CO] | rel-(R)-3-((3-(6- amino-2,5- difluoropyridin-3-yl)- 6-(tert- butylsulfonyl)imidazo [1,2-a]pyridin-7- yl)oxy)-2-fluoro-2- methylpropan-1-ol | [M + H] ⁺ = 473.0 ¹H NMR: (400 MHz, DMSO-d₆) δ ppm: 8.37 (d, J = 2.4 Hz, 1H), 7.77 (dd, J = 7.2, 10.0 Hz, 1H), 7.68 (s, 1H), 7.26 (s, 1H), 7.06 (s, 2H), 5.09 (t, J = 5.6 Hz, 1H), 4.28-4.21 (m, 2H), 3.64 (dd, J = 6.0, 18.4 Hz, 2H), 1.46-1.38 (m, 3H), 1.30 (s, 9H). |
| 262 | 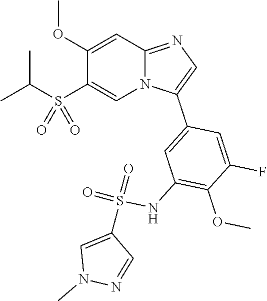 [SMILES: COc1cc2ncc(-c3cc(F)c(OC)c(NS(=O)(=O)c4cnn(C)c4)c3)n2cc1S(=O)(=O)C(C)C] | N-(3-fluoro-5-(6- (isopropylsulfonyl)-7- methoxyimidazo[1,2- a]pyridin-3-yl)-2- methoxyphenyl)-1- methyl-1H-pyrazole- 4-sulfonamide | [M + H] ⁺ = 538.1 ¹H NMR: (400 MHz, DMSO-d₆) δ ppm: 9.94 (s, 1H), 8.68 (s, 1H), 8.28 (s, 1H), 7.77 (s, 1H), 7.72 (s, 1H), 7.48- 7.31 (m, 2H), 7.31 (s, 1H), 3.99 (s, 3H), 3.84 (s, 3H), 3.74 (s, 3H), 3.75-3.62 (m, 1H), 1.23 (d, J = 6.8 Hz, 6H). |
| 263 | 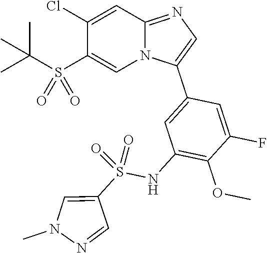 [SMILES: COc1c(F)cc(-c2cnc3cc(Cl)c(S(=O)(=O)C(C)(C)C)cn23)cc1NS(=O)(=O)c1cnn(C)c1] | N-(5-(6-(tert- butylsulfonyl)-7- chloroimidazo[1,2- a]pyridin-3-yl)-3- fluoro-2- methoxyphenyl)-1- methyl-1H-pyrazole- 4-sulfonamide | [M + H] ⁺ = 556.0 ¹H NMR: (400 MHz, DMSO-d₆) δ ppm: 10.15-9.70 (m, 1H), 8.85 (s, 1H), 8.24 (s, 1H), 8.09 (s, 1H), 7.95 (s, 1H), 7.74 (s, 1H), 7.49 (s, 1H), 7.34 (d, J = 11.6 Hz, 1H), 3.83 (s, 3H), 3.74 (d, J = 0.8 Hz, 3H), 1.39 (s, 9H). |
| 264 | 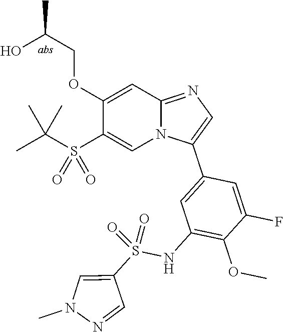 [SMILES: COc1c(F)cc(-c2cnc3cc(OC[C@H](C)O)c(S(=O)(=O)C(C)(C)C)cn23)cc1NS(=O)(=O)c1cnn(C)c1] | (S)-N-(5-(6-(tert- butylsulfonyl)-7-(2- hydroxypropoxy)imidazo [1,2-a]pyridin-3- yl)-3-fluoro-2- methoxyphenyl)-1- methyl-1H-pyrazole- 4-sulfonamide | [M + H] ⁺ = 596.1 ¹H NMR: (400 MHz, DMSO-d₆) δ ppm: 9.93 (m, 1H), 8.70 (s, 1H), 8.26 (s, 1H), 7.74 (d, J = 12.4 Hz, 2H), 7.44 (s, 1H), 7.32 ( d, J = 11.2 Hz, 1H), 7.27 (s, 1H), 4.79 (d, J = 3.6 Hz, 1H), 4.08-3.93 (m, 3H), 3.83 (s, 3H), 3.73 (s, 3H), 1.36 (s, 9H), 1.23 (d, J = 5.6 Hz, 3H). |
| Int. 157 | 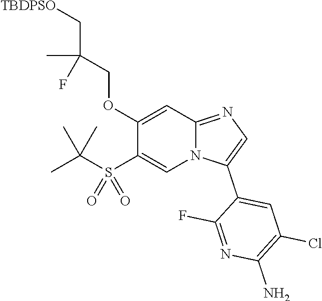 | 5-(7-(3-((tert- butyldiphenylsilyl) oxy)-2-fluoro-2- methylpropoxy)-6- (tert- butylsulfonyl)imidazo [1,2-a]pyridin-3-yl)- 3-chloro-6- fluoropyridin-2-amine | [M + H] ⁺ = 727.1 ¹H NMR: (400 MHz, CDCl₃-d) δ ppm: 8.47 (d, J = 2.4 Hz, 1H), 7.69-7.62 (m, 6H), 7.42-7.39 (m, 6H), 7.02 (s, 1H), 5.20 (s, 2H), 4.26-4.20 (m, 2H), 3.99-3.81 (m, 2H), 1.59 (br s, 3H), 1.38 (s, 9H), 1.08 (s, 9H). |
| 265 | 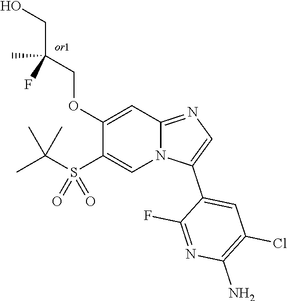 [SMILES: CC(C)(C)S(=O)(=O)c1cn2c(-c3cc(Cl)c(N)nc3F)cnc2cc1OC[C@@](C)(F)CO] | rel-(R)-3-((3-(6- amino-5-chloro-2- fluoropyridin-3-yl)-6- (tert- butylsulfonyl)imidazo [1,2-a]pyridin-7- yl)oxy)-2-fluoro-2- methylpropan-1-ol | [M + H] ⁺ = 489.0 ¹H NMR: (400 MHz, DMSO-d₆) δ ppm: 8.37 (br s, 1H), 7.93 (d, J = 8.0 Hz, 1H), 7.69 (s, 1H), 7.25 (br s, 1H), 7.12 (br s, 2H), 5.10 (br s, 1H), 4.25 (d, J = 19.6 Hz, 2H), 3.64 (d, J = 18.0 Hz, 2H), 1.50-1.39 (m, 3H), 1.30 (br s, 9H). |
| 266 | 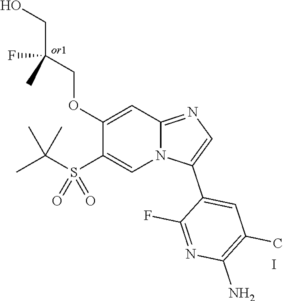 | rel-(R)-3-((3-(6- amino-5-chloro-2- fluoropyridin-3-yl)-6- (tert- butylsulfonyl)imidazo [1,2-a]pyridin-7- yl)oxy)-2-fluoro-2- methylpropan-1-ol | [M + H] ⁺ = 489.0 ¹H NMR: (400 MHz, DMSO-d₆) δ ppm: 8.37 (br s, 1H), 7.93 (d, J = 6.8 Hz, 1H), 7.69 (br s, 1H), 7.25 (br s, 1H), 7.12 (br s, 2H), 5.09 (br s, 1H), 4.27-4.22 (m, 2H), 3.64 (d, J = 16.4 Hz, 2H), 1.50-1.39 (m, 3H), 1.30 (br s, 9H). |
| 267 | 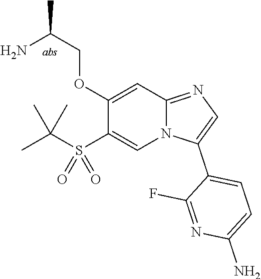 [SMILES: C[C@H](N)COc1cc2ncc(-c3ccc(N)nc3F)n2cc1S(=O)(=O)C(C)(C)C] | (S)-1-((3-(6-amino-2- fluoropyridin-3-yl)-6- (tert- butylsulfonyl)imidazo [1,2-a]pyridin-7- yl)oxy)propan-2-ol | [M + H] ⁺ = 423.1 ¹H NMR: (400 MHz, DMSO-d₆) δ ppm: 8.30 (d, J = 2.4 Hz, 1H), 7.70-7.59 (m, 2H), 7.23 (s, 1H), 6.78 (s, 2H), 6.49 (dd, J = 1.6, 8.0 Hz, 1H), 5.12-4.38 (m, 1H), 4.05-3.95 (m, 3H), 1.31 (s, 9H), 1.22 (d, J = 5.6 Hz, 3H). |
| 268 | 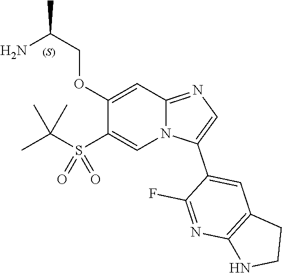 | (S)-1-((6-(tert- butylsulfonyl)-3-(6- fluoro-1H- pyrrolo[2,3-b]pyridin- 5-yl)imidazo[1,2- a]pyridin-7- yl)oxy)propan-2-ol | [M + H]⁺ = 447.1 ¹H NMR: (400 MHz, DMSO-d₆) δ ppm: 12.06 (br s, 1H), 8.39 (d, J = 2.8 Hz, 1H), 8.31 (d, J = 9.6 Hz, 1H), 7.77 (s, 1H), 7.57 (d, J = 3.6 Hz, 1H), 7.29 (s, 1H), 6.61 (d, J = 3.6 Hz, 1H), 4.81 (d, J = 3.6 Hz, 1H), 4.09- 3.96 (m, 3H), 1.31 (s, 9H), 1.23 (d, J = 5.6 Hz, 3H). |
| 269 | 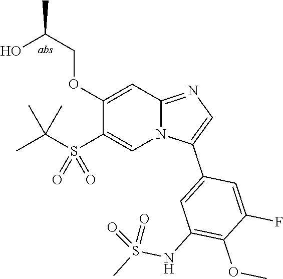 [SMILES: COc1c(F)cc(-c2cnc3cc(OC[C@H](C)O)c(S(=O)(=O)C(C)(C)C)cn23)cc1NS(C)(=O)=O] | (S)-N-(5-(6-(tert- butylsulfonyl)-7-(2- hydroxypropoxy) imidazo[1,2-a]pyridin-3- yl)-3-fluoro-2- methoxyphenyl) methanesulfonamide | [M + H] ⁺ = 530.0 ¹H NMR: (400 MHz, DMSO-d₆) δ ppm: 9.85 (s, 1H), 8.72 (s, 1H), 7.75 (s, 1H), 7.40 (s, 1H), 7.32 (d, J = 12.0 Hz, 1H), 7.26 (s, 1H), 4.79 (d, J = 2.4 Hz, 1H), 4.04-3.96 (m, 3H), 3.93 (s, 3H), 3.07 (s, 3H), 1.34 (s, 9H), 1.22 (d, J = 6.0 Hz, 3H). |
| 270 | 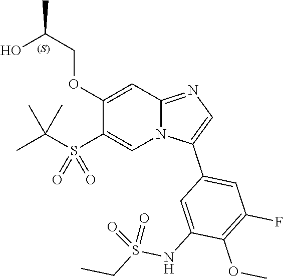 | (S)-N-(5-(6-(tert- butylsulfonyl)-7-(2- hydroxypropoxy) imidazo[1,2-a]pyridin- 3-yl)-3-fluoro-2- methoxyphenyl) ethanesulfonamide | [M + H] ⁺ = 544.1 ¹H NMR: (400 MHz, DMSO-d₆) δ ppm: 9.57 (s, 1H), 8.70 (s, 1H), 7.76 (s, 1H), 7.42 (s, 1H), 7.40-7.35 (m, 1H), 7.26 (s, 1H), 4.79 (d, J = 4.0 Hz, 1H), 4.06- 3.96 (m, 3H), 3.94 (d, J = 1.6 Hz, 3H), 3.24- 3.17 (m, 2H), 1.35 (s, 9H), 1.27 (t, J = 7.2 Hz, 3H), 1.22 (d, J = 5.6 Hz, 3H). |
| 271 | 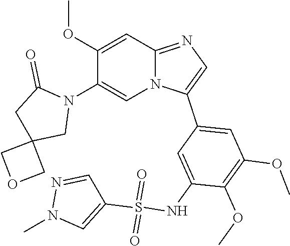 [SMILES: COc1cc2ncc(-c3cc(NS(=O)(=O)c4cnn(C)c4)c(OC)c(OC)c3)n2cc1N1CC2(COC2)CC1=O] | N-(2,3-dimethoxy-5- (7-methoxy-6-(7-oxo- 2-oxa-6- azaspiro[3.4]octan-6- yl)imidazo[1,2- a]pyridin-3- yl)phenyl)-1-methyl- 1H-pyrazole-4- sulfonamide | [M + H] ⁺ = 569.1 ¹H NMR: (400 MHz, DMSO-d₆) δ ppm: 9.62 (s, 1H), 8.39 (s, 1H), 8.21 (s, 1H), 7.71 (s, 1H), 7.59 (s, 1H), 7.23- 7.10 (m, 2H), 6.96 (s, 1H), 4.68-4.57 (m, 4H), 3.97 (s, 2H), 3.89 (s, 3H), 3.83 (d, J = 5.2 Hz, 6H), 3.59 (s, 3H), 2.81 (s, 2H). |
| Int. 158 | 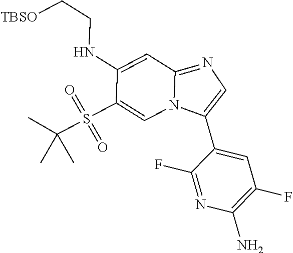 [SMILES: CC(C)(C)[Si](C)(C)OCCNc1cc2ncc(-c3cc(F)c(N)nc3F)n2cc1S(=O)(=O)C(C)(C)C] | 3-(6-amino-2,5- difluoropyridin-3-yl)- N-(2-((tert- butyldimethylsilyl) oxy)ethyl)-6-(tert- butylsulfonyl)imidazo [1,2-a]pyridin-7- amine | ¹H NMR: (400 MHz, DMSO-d₆) δ ppm: 8.19 (d, J = 2.0 Hz, 1H), 7.51 (s, 1H), 6.99 (s, 2H), 6.74 (s, 1H), 6.62 (s, 1H), 6.60-6.53 (m, 1H), 3.84 (t, J = 4.8 Hz, 2H), 3.27 (d, J = 5.6 Hz, 2H), 1.32 (s, 9H), 0.89 (s, 9H), 0.08 (s, 6H) |
| 272 | 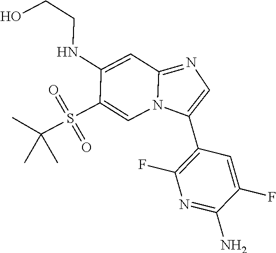 [SMILES: CC(C)(C)S(=O)(=O)c1cn2c(-c3cc(F)c(N)nc3F)cnc2cc1NCCO] | 2-((3-(6-amino-2,5- difluoropyridin-3-yl)- 6-(tert- butylsulfonyl)imidazo [1,2-a]pyridin-7- yl)amino)ethan-1-ol | [M + H] ⁺ = 426.0 ¹H NMR: (400 MHz, DMSO-d₆) δ ppm: 8.19 (d, J = 2.4 Hz, 1H), 7.71 (dd, J = 7.2, 10.0 Hz, 1H), 7.50 (s, 1H), 6.98 (s, 2H), 6.60 (s, 1H), 6.55 (t, J = 4.4 Hz, 1H), 4.91 (t, J = 4.8 Hz, 1H), 3.67-3.62 (m, 2H), 3.24-3.20 (m, 2H), 1.32 (s, 9H). |
| 273 | 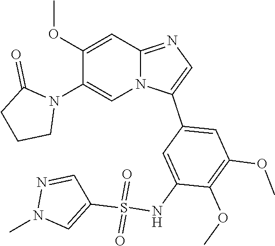 [SMILES: COc1cc2ncc(-c3cc(NS(=O)(=O)c4cnn(C)c4)c(OC)c(OC)c3)n2cc1N1CCCC1=O] | N-(2,3-dimethoxy-5- (7-methoxy-6-(2- oxopyrrolidin-1- yl)imidazo[1,2- a]pyridin-3- yl)phenyl)-1-methyl- 1H-pyrazole-4- sulfonamide | [M + H] ⁺ = 527.1 ¹H NMR: (400 MHz, DMSO) δ ppm: 9.63 (s, 1H), 8.37 (s, 1H), 8.22 (s, 1H), 7.72 (s, 1H), 7.59 (s, 1H), 7.13 (d, J = 12.4 Hz, 2H), 6.96 (s, 1H), 3.89 (s, 3H), 3.83 (d, J = 6.0 Hz, 6H), 3.69 (t, J = 6.4 Hz, 2H), 3.60 (s, 3H), 2.40 (t, J = 8.0 Hz, 2H), 2.16- 2.04 (m, 2H). |
| Int. 159 | 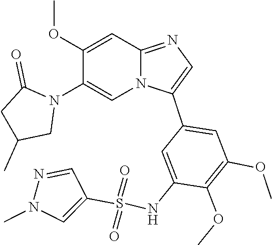 [SMILES: COc1cc2ncc(-c3cc(NS(=O)(=O)c4cnn(C)c4)c(OC)c(OC)c3)n2cc1N1CC(C)CC1=O] | N-(2,3-dimethoxy-5- (7-methoxy-6-(4- methyl-2- oxopyrrolidin-1- yl)imidazo[1,2- a]pyridin-3- yl)phenyl)-1-methyl- 1H-pyrazole-4- sulfonamide | [M + H] ⁺ = 541.1 ¹H NMR: (400 MHz, DMSO-d₆) δ ppm: 9.62 (s, 1H), 8.40 (s, 1H), 8.22 (s, 1H), 7.71 (s, 1H), 7.60 (s, 1H), 7.22- 7.11 (m, 2H), 6.98 (d, J = 1.6 Hz, 1H), 3.89 (s, 3H), 3.86-3.78 (m, 7H), 3.59 (s, 3H), 3.30-3.27 (m, 1H), 2.62-2.53 (m, 2H), 2.13-2.00 (m, 1H), 1.13 (d, J = 6.4 Hz, 3H). |
| 274 | 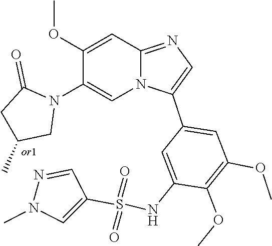 [SMILES: COc1cc2ncc(-c3cc(NS(=O)(=O)c4cnn(C)c4)c(OC)c(OC)c3)n2cc1N1C[C@H](C)CC1=O] | rel-(R)-N-(2,3- dimethoxy-5-(7- methoxy-6-(4-methyl- 2-oxopyrrolidin-1- yl)imidazo[1,2- a]pyridin-3- yl)phenyl)-1-methyl- 1H-pyrazole-4- sulfonamide | [M + H] ⁺ = 541.1 ¹H NMR: (400 MHz, DMSO-d₆) δ ppm: 9.62 (s, 1H), 8.40 (s, 1H), 8.22 (s, 1H), 7.71 (s, 1H), 7.60 (s, 1H), 7.22- 7.11 (m, 2H), 6.98 (d, J = 1.6 Hz, 1H), 3.89 (s, 3H), 3.86-3.78 (m, 7H), 3.59 (s, 3H), 3.30-3.27 (m, 1H), 2.62-2.53 (m, 2H), 2.13-2.00 (m, 1H), 1.13 (d, J = 6.4 Hz, 3H). |
| 275 | 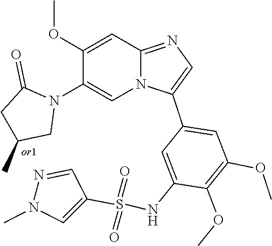 [SMILES: COc1cc2ncc(-c3cc(NS(=O)(=O)c4cnn(C)c4)c(OC)c(OC)c3)n2cc1N1C[C@@H](C)CC1=O] | rel-(R)-N-(2,3- dimethoxy-5-(7- methoxy-6-(4-methyl- 2-oxopyrrolidin-1- yl)imidazo[1,2- a]pyridin-3- yl)phenyl)-1-methyl- 1H-pyrazole-4- sulfonamide | [M + H] ⁺ = 541.1 ¹H NMR: (400 MHz, DMSO-d₆) δ ppm: 9.72-9.53 (m, 1H), 8.39 (s, 1H), 8.20 (s, 1H), 7.70 (s, 1H), 7.63-7.53 (m, 1H), 7.16-7.10 (m, 2H), 7.01-6.91 (m, 1H), 3.89 (s, 3H), 3.83 (d, J = 6.0 Hz, 7H), 3.59 (s, 3H), 3.30-3.27 (m, 1H), 2.62-2.58 (m, 2H), 2.10-2.01 (m, 1H), 1.13 (d, J = 6.4 Hz, 3H). |
| Int. 160 | 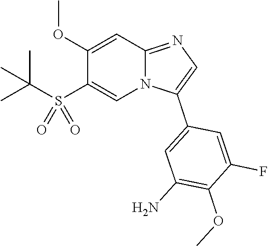 [SMILES: COc1cc2ncc(-c3cc(N)c(OC)c(F)c3)n2cc1S(=O)(=O)C(C)(C)C] | 5-(6-(tert- butylsulfonyl)-7- methoxyimidazo[1,2- a]pyridin-3-yl)-3- chloro-2- methoxyaniline | ¹H NMR: (400 MHz, CDCl₃) δ ppm: 8.84 (s, 1H), 7.57 (s, 1H), 7.01 (s, 1H), 6.83 (d, J = 2.0 Hz, 1H), 6.74 (d, J = 2.0 Hz, 1H), 3.97 (s, 3H), 3.92 (s, 3H), 1.41 (s, 9H) |
| Int. 161 | 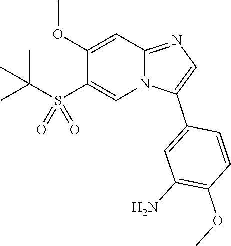 [SMILES: COc1ccc(-c2cnc3cc(OC)c(S(=O)(=O)C(C)(C)C)cn23)cc1N] | 5-(6-(tert- butylsulfonyl)-7- methoxyimidazo[1,2- a]pyridin-3-yl)-2- methoxyaniline | [M + H] ⁺ = 389.9 |
| 276 | 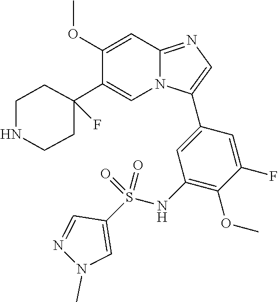 [SMILES: COc1cc2ncc(-c3cc(F)c(OC)c(NS(=O)(=O)c4cnn(C)c4)c3)n2cc1C1(F)CCNCC1] | N-(3-fluoro-5-(6-(4- fluoropiperidin-4-yl)- 7- methoxyimidazo[1,2- a]pyridin-3-yl)-2- methoxyphenyl)-1- methyl-1H-pyrazole- 4-sulfonamide | [M + H] ⁺ = 533.2 ¹H NMR: (400 MHz, DMSO-d₆) δ ppm: 8.29 (s, 1H), 8.28 (s, 1H), 8.14 (s, 1H), 7.66 (s, 1H), 7.55 (s, 1H), 7.32 (s, 1H), 7.21-7.13 (m, 2H), 3.91 (s, 3H), 3.82 (s, 3H), 3.73 (s, 3H), 3.20-3.12 (m, 2H), 3.02 (m, 2H), 2.69-2.60 (m, 1H), 2.59-2.52 (m, 1H), 2.02-1.88 (m, 2H) |
| Int. 162 | 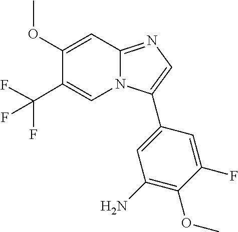 [SMILES: COc1cc2ncc(-c3cc(N)c(OC)c(F)c3)n2cc1C(F)(F)F] | 3-fluoro-2-methoxy- 5-(7-methoxy-6- (trifluoromethyl)imidazo [1,2-a]pyridin-3- yl)aniline | ¹H NMR: (400 MHz, DMSO-d₆) δ ppm: 8.60 (s, 1H), 7.62 (s, 1H), 7.27 (s, 1H), 6.76-6.63 (m, 2H), 5.47 (s, 2H), 3.96 (s, 3H), 3.80 (s, 3H) |

The following compounds were prepared following procedures analogous to that described for Example 11.

| Ex. | | | |
|---|---|---|---|
| No. | Structure | Name | LCMS, ¹H NMR |
| 277 | 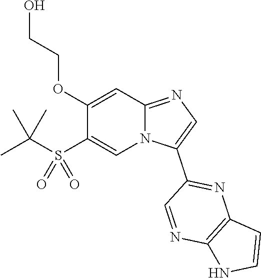 [SMILES: CC(C)(C)S(=O)(=O)c1cn2c(-c3cnc4[nH]ccc4n3)cnc2cc1OCCO] | 2-((6-(tert- butylsulfonyl)-3- (5H-pyrrolo[2,3- b]pyrazin-2- yl)imidazo[1,2- a]pyridin-7- yl)oxy)ethan-1-ol | [M + H]⁺ = 416.0 ¹H NMR (400 MHZ, DMSO-d₆) δ ppm 12.21 (s, 1H), 10.43 (s, 1H), 8.94 (s, 1H), 8.45 (s, 1H), 7.91 (t, J = 3.2 Hz, 1H), 7.34 (s, 1H), 6.62 (dd, J = 3.6, 1.6 Hz, 1H), 4.98-4.72 (m, 1H), 4.21 (t, J = 4.8 Hz, 2H), 3.79 (t, J = 4.8 Hz, 2H), 1.36 (s, 9H). |
| 278 | 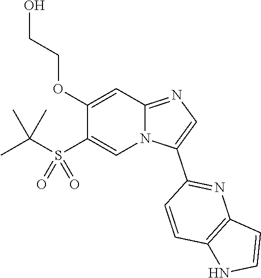 [SMILES: CC(C)(C)S(=O)(=O)c1cn2c(-c3ccc4[nH]ccc4n3)cnc2cc1OCCO] | 2-((6-(tert- butylsulfonyl)-3- (1H-pyrrolo[3,2- b]pyridin-5- yl)imidazo[1,2- a]pyridin-7- yl)oxy)ethan-1-ol | [M + H]⁺ = 415.0. ¹H NMR: (400 MHZ, DMSO-d₆) δ ppm: 11.47- 11.45 (m, 1H), 10.73 (s, 1H), 8.38 (s, 1H), 8.29 (s, 1H), 7.90 (d, J = 8.8 Hz, 1H), 7.74 (d, J = 8.8 Hz, 1H), 7.68 (t, J = 2.8 Hz, 1H), 7.29 (s, 1H), 6.56- 6.48 (m, 1H), 4.86-4.78 (m, 1H), 4.20 (t, J = 4.8 Hz, 2H), 3.79 (t, J = 4.8 Hz, 2H), 1.37 (s, 9H). |

The following compounds were prepared following procedures analogous to that described for Example 126. Examples prepared using intermediates or building blocks containing Boc, TITP, TBS, dioxolane or other similar protecting groups were subjected to protecting group removal under acidic conditions (generally with HCl or TFA) prior to purification of the final product, as appropriate. All anilines whose syntheses are not described herein are made according to known procedures.

| Ex. | | | |
|---|---|---|---|
| No. | Structure | Name | LCMS, ¹H NMR |
| 279 | 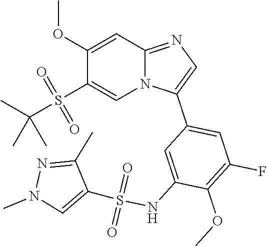 [SMILES: COc1cc2ncc(-c3cc(F)c(OC)c(NS(=O)(=O)c4cn(C)nc4C)c3)n2cc1S(=O)(=O)C(C)(C)C] | N-(5-(6-(tert- butylsulfonyl)-7- methoxyimidazo[1,2- a]pyridin-3-yl)-3-fluoro- 2-methoxyphenyl)-1,3- dimethyl-1H-pyrazole-4- sulfonamide | [M + H]⁺ = 566.0 ¹H NMR: (400 MHZ, DMSO-d₆) δ ppm: 9.94 (s, 1H), 8.69 (s, 1H), 8.14 (s, 1H), 7.73 (s, 1H), 7.41 (s, 1H), 7.36 (d, J = 12.4 Hz, 1H), 7.28 (s, 1H), 3.94 (s, 3H), 3.72 (s, 6H), 2.22 (s, 3H), 1.33 (s, 9H). |
| 280 | 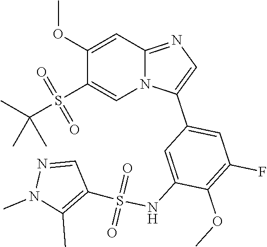 [SMILES: COc1cc2ncc(-c3cc(F)c(OC)c(NS(=O)(=O)c4cnn(C)c4C)c3)n2cc1S(=O)(=O)C(C)(C)C] | N-(5-(6-(tert- butylsulfonyl)-7- methoxyimidazo[1,2- a]pyridin-3-yl)-3-fluoro- 2-methoxyphenyl)-1,5- dimethyl-1H-pyrazole-4- sulfonamide | [M + H]⁺ = 566.0 ¹H NMR: (400 MHZ, DMSO-d₆) δ ppm: 9.94 (s, 1H), 8.70 (s, 1H), 7.69 (d, J = 12.4 Hz, 2H), 7.40 (s, 1H), 7.34 (d, J = 12.0 Hz, 1H), 7.28 (s, 1H), 3.94 (s, 3H), 3.74-3.69 (m, 6H), 2.36 (s, 3H), 1.34 (s, 9H). |
| 281 | 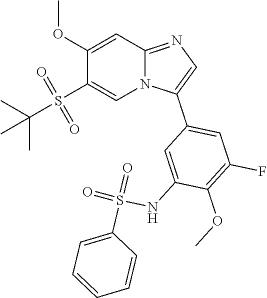 [SMILES: COc1cc2ncc(-c3cc(F)c(OC)c(NS(=O)(=O)c4ccccc4)c3)n2cc1S(=O)(=O)C(C)(C)C] | N-(5-(6-(tert- butylsulfonyl)-7- methoxyimidazo[1,2- a]pyridin-3-yl)-3-fluoro- 2-methoxyphenyl)benzenes ulfonamide | [M + H]⁺ = 548.0 ¹H NMR: (400 MHZ, DMSO-d₆) δ ppm: 10.19 (d, J = 1.6 Hz, 1H), 8.70 (s, 1H), 7.87-7.85 (m, 2H), 7.70 (s, 1H), 7.63- 7.62 (m, 1H), 7.59-7.55 (m, 2H), 7.42 (s, 1H), 7.34 (dd, J = 1.6, 12.0 Hz, 1H), 7.28 (s, 1H), 3.94 (s, 3H), 3.57 (d, J = 1.6 Hz, 3H), 1.35 (s, 9H). |
| 282 | 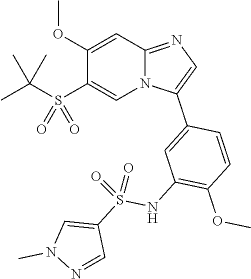 [SMILES: COc1ccc(-c2cnc3cc(OC)c(S(=O)(=O)C(C)(C)C)cn23)cc1NS(=O)(=O)c1cnn(C)c1] | N-(5-(6-(tert- butylsulfonyl)-7- methoxyimidazo[1,2- a]pyridin-3-yl)-2- methoxyphenyl)-1- methyl-1H-pyrazole-4- sulfonamide | [M + H]⁺ = 534.0 ¹H NMR: (400 MHZ, DMSO-d₆) δ ppm: 9.45 (s, 1H), 8.63 (s, 1H), 8.18 (s, 1H), 7.68 (s, 1H), 7.64 (s, 1H), 7.49 (d, J = 0.8 Hz, 1H), 7.34 (dd, J = 2.0, 10.4 Hz, 1H), 7.24 (s, 1H), 7.17 (d, J = 8.8 Hz, 1H), 3.93 (s, 3H), 3.82 (s, 3H), 3.73 (s, 3H), 1.32 (s, 9H). |
| 283 | 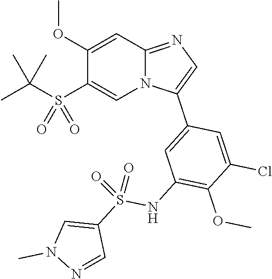 [SMILES: COc1cc2ncc(-c3cc(Cl)c(OC)c(NS(=O)(=O)c4cnn(C)c4)c3)n2cc1S(=O)(=O)C(C)(C)C] | N-(5-(6-(tert- butylsulfonyl)-7- methoxyimidazo[1,2- a]pyridin-3-yl)-3-chloro- 2-methoxyphenyl)-1- methyl-1H-pyrazole-4- sulfonamide | [M + H]⁺ = 568.0 ¹H NMR: (400 MHZ, DMSO-d₆) δ ppm: 10.00 (s, 1H), 8.69 (s, 1H), 8.29 (s, 1H), 7.87-7.69 (m, 2H), 7.62 (d, J = 2.0 Hz, 1H), 7.47 (s, 1H), 7.27 (s, 1H), 3.94 (s, 3H), 3.83 (s, 3H), 3.66 (s, 3H), 1.33 (s, 9H). |
| 284 | 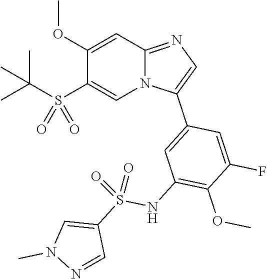 | N-(3-(6-(tert- butylsulfonyl)-7- methoxyimidazo[1,2- a]pyridin-3-yl)-5- fluorophenyl)-1-methyl- 1H-pyrazole-4- sulfonamide | [M + H]⁺ = 522.0 ¹H NMR: (400 MHZ, DMSO-d₆) δ ppm: 10.72 (s, 1H), 8.70 (s, 1H), 8.35 (s, 1H), 7.81 (s, 1H), 7.79 (s, 1H), 7.29 (s, 1H), 7.21 (s, 1H), 7.18 (d, J = 9.2 Hz, 1H), 6.98 (d, J = 10.8 Hz, 1H), 3.94 (s, 3H), 3.84 (s, 3H), 1.33 (s, 9H). |
| 285 | 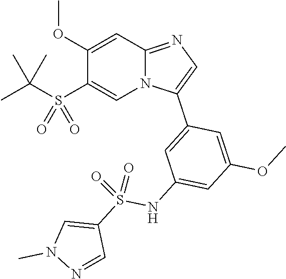 [SMILES: COc1cc(NS(=O)(=O)c2cnn(C)c2)cc(-c2cnc3cc(OC)c(S(=O)(=O)C(C)(C)C)cn23)c1] | N-(3-(6-(tert- butylsulfonyl)-7- methoxyimidazo[1,2- a]pyridin-3-yl)-5- methoxyphenyl)-1- methyl-1H-pyrazole-4- sulfonamide | [M + H]⁺ = 534.0 ¹H NMR: (400 MHZ, DMSO-d₆) δ ppm: 10.40 (s, 1H), 8.72 (s, 1H), 8.29 (s, 1H), 7.76 (s, 1H), 7.73 (s, 1H), 7.27 (s, 1H), 6.98 (s, 1H), 6.87 (s, 1H), 6.76 (s, 1H), 3.93 (s, 3H), 3.83 (s, 3H), 3.75 (s, 3H), 1.32 (s, 9H). |
| 286 | 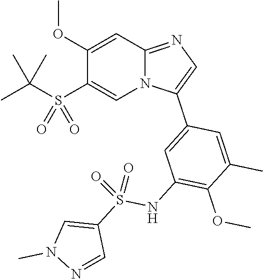 [SMILES: COc1cc2ncc(-c3cc(C)c(OC)c(NS(=O)(=O)c4cnn(C)c4)c3)n2cc1S(=O)(=O)C(C)(C)C] | N-(5-(6-(tert- butylsulfonyl)-7- methoxyimidazo[1,2- a]pyridin-3-yl)-2- methoxy-3- methylphenyl)-1-methyl- 1H-pyrazole-4- sulfonamide | [M + H]⁺ = 548.0 ¹H NMR: (400 MHZ, DMSO-d₆) δ ppm: 9.64 (brs, 1H), 8.70 (s, 1H), 8.28 (s, 1H), 7.78 (s, 1H), 7.67 (s, 1H), 7.49 (d, J = 1.6 Hz, 1H), 7.27 (s, 1H), 7.17 (s, 1H), 3.94 (s, 3H), 3.82 (s, 3H), 3.56 (s, 3H), 2.25 (s, 3H), 1.33 (s, 9H). |
| 287 | 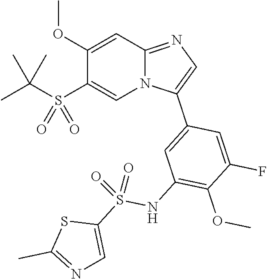 [SMILES: COc1cc2ncc(-c3cc(F)c(OC)c(NS(=O)(=O)c4cnc(C)s4)c3)n2cc1S(=O)(=O)C(C)(C)C] | N-(5-(6-(tert- butylsulfonyl)-7- methoxyimidazo[1,2- a]pyridin-3-yl)-3-fluoro- 2-methoxyphenyl)-2- methylthiazole-5- sulfonamide | [M + H]⁺ = 569.2 ¹H NMR: (400 MHZ, DMSO-d₆) δ ppm: 10.69- 10.44 (m, 1H), 8.71 (s, 1H), 8.04 (s, 1H), 7.71 (s, 1H), 7.41 (s, 1H), 7.34- 7.27 (m, 2H), 3.94 (s, 3H), 3.74 (d, J = 1.2 Hz, 3H), 2.67 (s, 3H), 1.33 (s, 9H). |
| 288 | 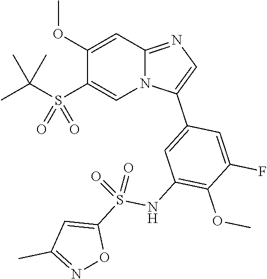 [SMILES: COc1cc2ncc(-c3cc(F)c(OC)c(NS(=O)(=O)c4cc(C)no4)c3)n2cc1S(=O)(=O)C(C)(C)C] | N-(5-(6-(tert- butylsulfonyl)-7- methoxyimidazo[1,2- a]pyridin-3-yl)-3-fluoro- 2-methoxyphenyl)-3- methylisoxazole-5- sulfonamide | [M + H]⁺ = 553.0 ¹H NMR: (400 MHZ, DMSO-d₆) δ ppm: 11.49- 10.86 (m, 1H), 8.68 (s, 1H), 7.74 (s, 1H), 7.44 (d, J = 12.0 Hz, 1H), 7.36 (s, 1H), 7.28 (s, 1H), 6.99 (s, 1H), 3.94 (s, 3H), 3.76 (d, J = 1.6 Hz, 3H), 2.28 (s, 3H), 1.32 (s, 9H). |
| 289 | 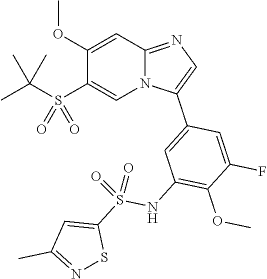 [SMILES: COc1cc2ncc(-c3cc(F)c(OC)c(NS(=O)(=O)c4cc(C)ns4)c3)n2cc1S(=O)(=O)C(C)(C)C] | N-(5-(6-(tert- butylsulfonyl)-7- methoxyimidazo[1,2- a]pyridin-3-yl)-3-fluoro- 2-methoxyphenyl)-3- methylisothiazole-5- sulfonamide | [M + H]⁺ = 569.0 ¹H NMR: (400 MHZ, DMSO-d₆) δ ppm: 10.98- 10.68 (m, 1H), 8.69 (s, 1H), 7.74 (s, 1H), 7.62 (s, 1H), 7.48-7.39 (m, 2H), 7.28 (s, 1H), 3.94 (s, 3H), 3.70 (d, J = 1.6 Hz, 3H), 2.44 (s, 3H), 1.33 (s, 9H). |
| 290 | 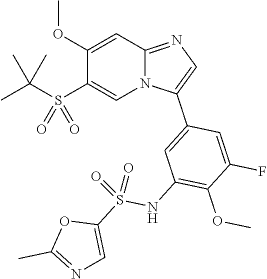 [SMILES: COc1cc2ncc(-c3cc(F)c(OC)c(NS(=O)(=O)c4cnc(C)o4)c3)n2cc1S(=O)(=O)C(C)(C)C] | N-(5-(6-(tert- butylsulfonyl)-7- methoxyimidazo[1,2- a]pyridin-3-yl)-3-fluoro- 2-methoxyphenyl)-2- methyloxazole-5- sulfonamide | [M + H]⁺ = 553.0 ¹H NMR: (400 MHZ, DMSO-d₆) δ ppm: 10.92- 10.68 (m, 1H), 8.68 (s, 1H), 7.75 (s, 1H), 7.72- 7.66 (m, 1H), 7.49-7.41 (m, 1H), 7.39-7.34 (m, 1H), 7.28 (s, 1H), 3.94 (s, 3H), 3.80 (s, 3H), 2.50 (s, 3H), 1.33 (s, 9H). |
| 291 | 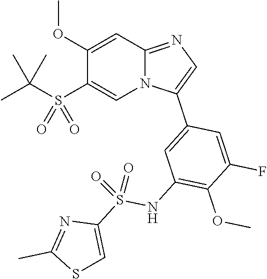 [SMILES: COc1cc2ncc(-c3cc(F)c(OC)c(NS(=O)(=O)c4csc(C)n4)c3)n2cc1S(=O)(=O)C(C)(C)C] | N-(5-(6-(tert- butylsulfonyl)-7- methoxyimidazo[1,2- a]pyridin-3-yl)-3-fluoro- 2-methoxyphenyl)-2- methylthiazole-4- sulfonamide | [M + H]⁺ = 569.1 ¹H NMR: (400 MHZ, DMSO-d₆) δ ppm: 10.47- 10.08 (m, 1H), 8.68 (s, 1H), 8.25 (s, 1H), 7.69 (s, 1H), 7.43 (s, 1H), 7.38- 7.29 (m, 1H), 7.27 (s, 1H), 3.93 (s, 3H), 3.76 (s, 3H), 2.66 (s, 3H), 1.32 (s, 9H). |
| 292 | 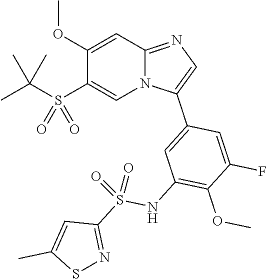 [SMILES: COc1cc2ncc(-c3cc(F)c(OC)c(NS(=O)(=O)c4cc(C)sn4)c3)n2cc1S(=O)(=O)C(C)(C)C] | N-(5-(6-(tert- butylsulfonyl)-7- methoxyimidazo[1,2- a]pyridin-3-yl)-3-fluoro- 2-methoxyphenyl)-5- methylisothiazole-3- sulfonamide | [M + H]⁺ = 569.1 ¹H NMR: (400 MHZ, DMSO-d₆) δ ppm: 8.70 (s, 1H), 8.13 (s, 0.40), 7.64 (s, 1H), 7.42 (s, 1H), 7.38 (s, 1H), 7.25 (s, 1H), 7.12- 7.03 (m, 1H), 3.93 (s, 3H), 3.71 (s, 3H), 2.39 (s, 3H), 1.32 (s, 9H). |
| 293 | 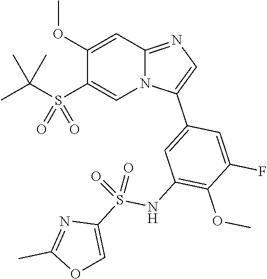 [SMILES: COc1cc2ncc(-c3cc(F)c(OC)c(NS(=O)(=O)c4coc(C)n4)c3)n2cc1S(=O)(=O)C(C)(C)C] | N-(5-(6-(tert- butylsulfonyl)-7- methoxyimidazo[1,2- a]pyridin-3-yl)-3-fluoro- 2-methoxyphenyl)-2- methyloxazole-4- sulfonamide | [M + H]⁺ = 553.0 ¹H NMR: (400 MHZ, DMSO-d₆) δ ppm: 10.60- 10.40 (m, 1H), 8.68 (s, 1H), 8.64 (s, 1H), 8.14 (s, 0.20H), 7.73 (s, 1H), 7.44 (s, 1H), 7.35-7.32 (m, 1H), 7.28 (s, 1H), 3.93 (s, 3H), 3.81 (s, 3H), 2.44 (s, 3H), 1.32 (s, 9H). |
| 294 | 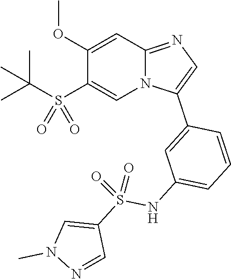 [SMILES: COc1cc2ncc(-c3cccc(NS(=O)(=O)c4cnn(C)c4)c3)n2cc1S(=O)(=O)C(C)(C)C] | N-(3-(6-(tert- butylsulfonyl)-7- methoxyimidazo[1,2- a]pyridin-3-yl)phenyl)-1- methyl-1H-pyrazole-4- sulfonamide | [M + H]⁺ = 504.0 ¹H NMR: (400 MHZ, DMSO-d₆) δ ppm: 10.41 (s, 1H), 8.69 (s, 1H), 8.28 (s, 1H), 7.74 (d, J = 9.6 Hz, 2H), 7.51-7.43 (m, 1H), 7.39 (s, 1H), 7.32- 7.26 (m, 2H), 7.20 (d, J = 8.0 Hz, 1H), 3.94 (s, 3H), 3.83 (s, 3H), 1.32 (s, 9H). |
| 295 | 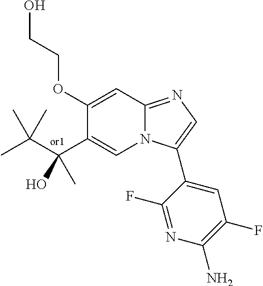 [SMILES: CC(C)(C)[C@](C)(O)c1cn2c(-c3cc(F)c(N)nc3F)cnc2cc1OCCO] | rel-(S)-2-(3-(6-amino- 2,5-difluoropyridin-3-yl)- 7-(2-hydroxyethoxy)imidazo [1,2-a]pyridin-6-yl)-3,3- dimethylbutan-2-ol | [M + H]⁺ = 407.1 ¹H NMR: (400 MHZ, DMSO-d₆) δ ppm: 8.11 (s, 1H), 7.71-7.64 (m, 1H), 7.44 (s, 1H), 6.94 (s, 3H), 4.90-4.84 (m, 2H), 4.10- 4.04 (m, 1H), 4.02-3.95 (m, 1H), 3.77 (q, J = 4.8 Hz, 2H), 1.65 (s, 3H), 0.88 (s, 9H). |
| 296 | 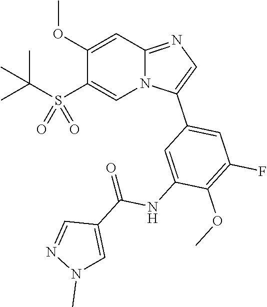 [SMILES: COc1cc2ncc(-c3cc(F)c(OC)c(NC(=O)c4cnn(C)c4)c3)n2cc1S(=O)(=O)C(C)(C)C] | N-(5-(6-(tert- butylsulfonyl)-7- methoxyimidazo[1,2- a]pyridin-3-yl)-3-fluoro- 2-methoxyphenyl)-1- methyl-1H-pyrazole-4- carboxamide | [M + H]⁺ = 516.1 ¹H NMR: (400 MHZ, DMSO-d₆) δ ppm: 9.44 (s, 1H), 8.74 (s, 1H), 8.38 (s, 1H), 8.02 (s, 1H), 7.90 (s, 1H), 7.76 (s, 1H), 7.41 (dd, J = 1.6, 12.0 Hz, 1H), 7.27 (s, 1H), 3.96 (d, J = 1.2 Hz, 3H), 3.94 (s, 3H), 3.90 (s, 3H), 1.33 (s, 9H). |
| 297 | 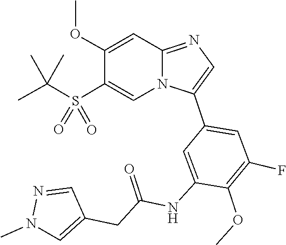 [SMILES: COc1cc2ncc(-c3cc(F)c(OC)c(NC(=O)Cc4cnn(C)c4)c3)n2cc1S(=O)(=O)C(C)(C)C] | N-(5-(6-(tert- butylsulfonyl)-7- methoxyimidazo[1,2- a]pyridin-3-yl)-3-fluoro- 2-methoxyphenyl)-2-(1- methyl-1H-pyrazol-4- yl)acetamide | [M + H]⁺ = 530.1 ¹H NMR: ES20817-730- PIA (400 MHZ, DMSO- d₆) δ ppm: 9.59 (s, 1H), 8.73 (s, 1H), 8.16 (s, 1H), 7.73 (s, 1H), 7.59 (s, 1H), 7.35 (s, 1H), 7.34-7.29 (m, 1H), 7.26 (s, 1H), 3.93 (s, 3H), 3.91 (s, 3H), 3.79 (s, 3H), 3.62 (s, 2H), 1.32 (s, 9H). |
| 298 | 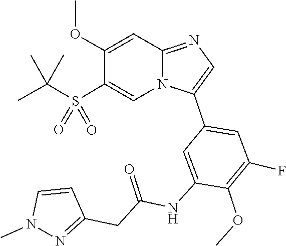 [SMILES: COc1cc2ncc(-c3cc(F)c(OC)c(NC(=O)Cc4ccn(C)n4)c3)n2cc1S(=O)(=O)C(C)(C)C] | N-(5-(6-(tert- butylsulfonyl)-7- methoxyimidazo[1,2- a]pyridin-3-yl)-3-fluoro- 2-methoxyphenyl)-2-(1- methyl-1H-pyrazol-3- yl)acetamide | [M + H]⁺ = 530.2 ¹H NMR: (400 MHZ, DMSO-d₆) δ ppm: 9.81 (s, 1H), 8.73 (s, 1H), 8.23 (s, 1H), 7.73 (s, 1H), 7.64 (d, J = 2.0 Hz, 1H), 7.32 (dd, J = 2.0, 12.0 Hz, 1H), 7.27 (s, 1H), 6.19 (d, J = 2.0 Hz, 1H), 3.93 (s, 6H), 3.83 (s, 3H), 3.76 (s, 2H), 1.32 (s, 9H). |

### Example 299

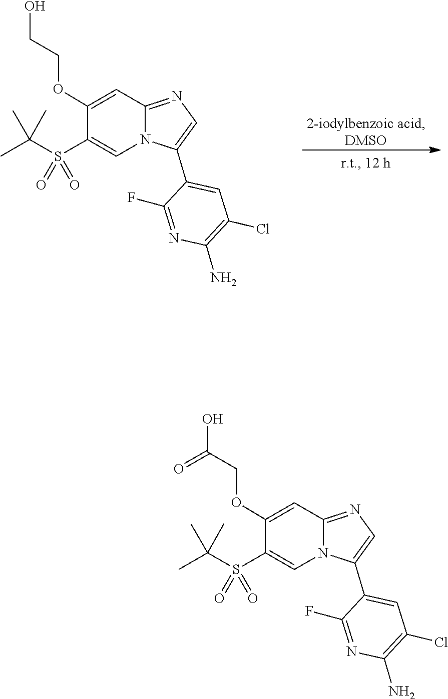 [SMILES: CC(C)(C)S(=O)(=O)c1cn2c(-c3cc(Cl)c(N)nc3F)cnc2cc1OCCO ; "2-iodylbenzoic acid,DMSO" ; "r.t., 12 h" ; CC(C)(C)S(=O)(=O)c1cn2c(-c3cc(Cl)c(N)nc3F)cnc2cc1OCC(=O)O]

2-((3-(6-amino-5-chloro-2-fluoropyridin-3-yl)-6-(tert-butylsulfonyl)imidazo[1,2-a]pyridin-7-yl)oxy)acetic acid: A solution of 2-((3-(6-amino-5-chloro-2-fluoropyridin-3-yl)-6-(tert-butylsulfonyl)imidazo[1,2-a]pyridin-7-yl)oxy)ethan-1-ol (100 mg, 226 μmol, 1 eq) in DMSO (10 mL) was added 2-iodylbenzoic acid (329 mg, 1.17 mmol, 5.2 eq). The mixture was stirred at 25° C. for 12 h The reaction mixture was diluted with H₂O (5 mL) and extracted with EtOAc (5 mL×3). The combined organic layers were washed with brine (10 mL), dried over Na₂SO₄, filtered and concentrated under reduced pressure to yield a residue, which was purified by prep-TLC and prep-HPLC (water (FA)—ACN) to yield the title compound (2.0 mg, 4.20 μmol, 1.86% yield, 97.4% purity, 0.15 HCOOH) as a yellow solid. [M+H]⁺=457.0. ¹HNMR: (400 MHz, DMSO-d₆) δ ppm: 8.37 (d, J=2.4 Hz, 1H), 8.16 (s, 0.15H), 7.93 (d, J=8.4 Hz, 1H), 7.67 (s, 1H), 7.15-7.09 (m, 2H), 6.99 (s, 1H), 4.76-4.60 (m, 2H), 1.34 (s, 9H).

### Example 300

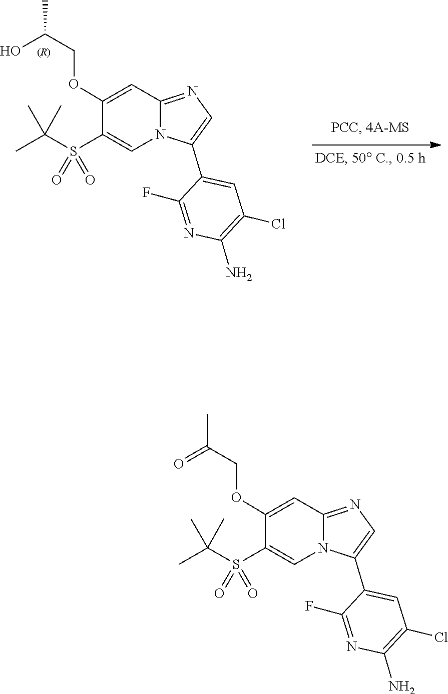

1-((3-(6-amino-5-chloro-2-fluoropyridin-3-yl)-6-(tert-butylsulfonyl)imidazo[1,2-a]pyridin-7-yl)oxy)propan-2-one: To a solution of (R)-1-((3-(6-amino-5-chloro-2-fluoropyridin-3-yl)-6-(tert-butylsulfonyl)imidazo[1,2-a]pyridin-7-yl)oxy)propan-2-ol (30 mg, 59.1 μmol, 1 eq) in DCE (3 mL) was added PCC (162 mg, 751 μmol, 12.7 eq) and 4A-MS (300 mg). The mixture was stirred at 50° C. for 0.5 h. The mixture was filtered and concentrated to give a residue, which was diluted with H₂O (10 mL) and extracted with EtOAc (10 mL×3). The combined organic layers were washed with brine (10 mL), dried over Na₂SO₄, filtered, and concentrated to give a residue, which was purified by prep-HPLC (water (FA)—ACN) to yield the title compound (3.32 mg, 6.92 μmol, 11.7% yield, 96.7% purity, 0.2 HCOOH) as a yellow solid. [M+H]⁺=454.9. ¹H NMR (400 MHz, DMSO-d₆) δ ppm:8.45 (s, 0.20H) 8.39 (d, J=2.4 Hz, 1H), 7.94 (d, J=8.8 Hz, 1H), 7.69 (s, 1H), 7.20 (s, 1H), 7.13 (s, 2H), 5.07 (s, 2H), 2.24 (s, 3H), 1.33 (s, 9H).

### Example 301

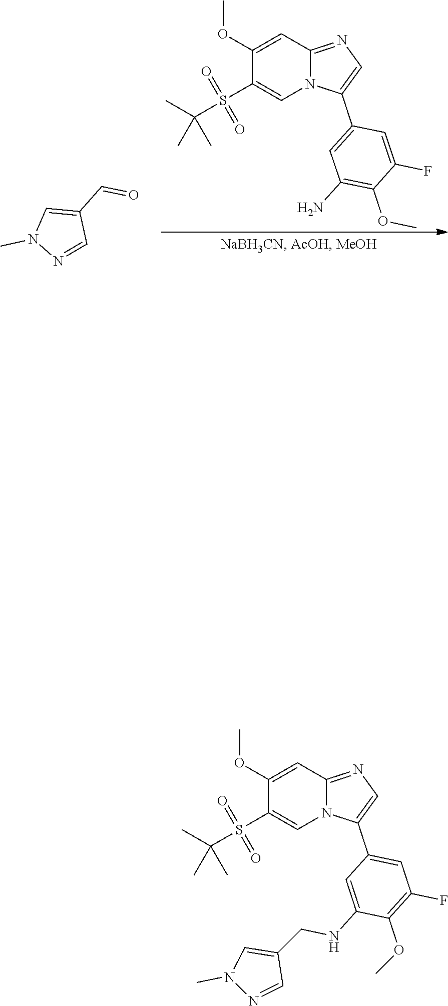

5-(6-(tert-butylsulfonyl)-7-methoxyimidazo[1,2-a]pyridin-3-yl)-3-fluoro-2-methoxy-N-((1-methyl-1H-pyrazol-4-yl)methyl)aniline: A mixture of 1-methyl-1H-pyrazole-4-carbaldehyde (5 mg, 45.4 μmol, 1 eq), and 5-(6-(tert-butylsulfonyl)-7-methoxyimidazo[1,2-a]pyridin-3-yl)-3-fluoro-2-methoxyaniline (30.8 mg, 68.1 μmol, 1.5 eq) in MeOH (0.5 mL) was added AcOH (2.73 mg, 45.4 μmol, 2.60 μL, 1 eq). The mixture was stirred at 25° C. for 10 min, and then NaBH₃CN (4.28 mg, 68.1 μmol, 1.5 eq) was added. The mixture was stirred at 25° C. for 3 h. The reaction mixture was partitioned between EtOAc (20 mL) and H₂O (10 mL). The organic phase was separated, washed with brine (10 mL×3), dried over Na₂SO₄, filtered, and concentrated under reduced pressure to give a residue, which was purified by prep-HPLC (water (FA)—ACN) to give the title compound (7.6 mg, 14.9 μmol, 32.9% yield, 98.4% purity) as a white solid. [M+H]⁺=502.1. ¹H NMR (400 MHz, DMSO-d₆) δ ppm: 8.75 (s, 1H), 7.68 (s, 1H), 7.59 (s, 1H), 7.36 (s, 1H), 7.25 (s, 1H), 6.71-6.64 (m, 2H), 5.96 (t, J=6.4 Hz, 1H), 4.18 (d, J=6.0 Hz, 2H), 3.92 (s, 3H), 3.82 (s, 3H), 3.75 (s, 3H), 1.31 (s, 9H).

The following compounds were prepared according to procedures analogous to that described for Example 304.

| Ex. | | | |
|---|---|---|---|
| No. | Structure | Name | LCMS, ¹H NMR |
| 302 | 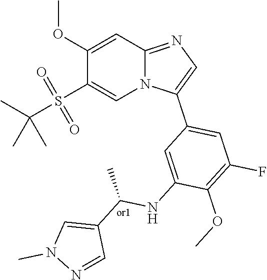 [SMILES: COc1cc2ncc(-c3cc(F)c(OC)c(N[C@@H](C)c4cnn(C)c4)c3)n2cc1S(=O)(=O)C(C)(C)C] | rel-(R)-5-(6-(tert- butylsulfonyl)-7- methoxyimidazo[1,2- a]pyridin-3-yl)-3-fluoro- 2-methoxy-N-(1-(1- methyl-1H-pyrazol-4- yl)ethyl)aniline | [M + H]⁺ = 516.1 1H NMR: (400 MHZ, DMSO-d₆) δ ppm: 8.73 (s, 1H), 7.66 (s, 1H), 7.58 (s, 1H), 7.36 (s, 1H), 7.24 (s, 1H), 6.71-6.64 (m, 2H), 5.49 (d, J = 8.4 Hz, 1H), 4.70-4.54 (m, 1H), 3.92 (s, 3H), 3.83 (s, 3H), 3.75 (s, 3H), 1.48 (d, J = 6.4 Hz, 3H), 1.31 (s, 9H). |
| 303 | 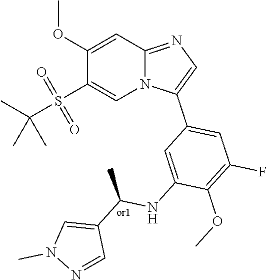 [SMILES: COc1cc2ncc(-c3cc(F)c(OC)c(N[C@H](C)c4cnn(C)c4)c3)n2cc1S(=O)(=O)C(C)(C)C] | rel-(R)-5-(6-(tert- butylsulfonyl)-7- methoxyimidazo[1,2- a]pyridin-3-yl)-3-fluoro- 2-methoxy-N-(1-(1- methyl-1H-pyrazol-4- yl)ethyl)aniline | [M + H]⁺ = 516.1 1H NMR: (400 MHZ, DMSO-d₆) δ ppm: 8.73 (s, 1H), 7.66 (s, 1H), 7.58 (s, 1H), 7.36 (s, 1H), 7.24 (s, 1H), 6.80-6.60 (m, 2H), 5.49 (d, J = 8.4 Hz, 1H), 4.61 (t, J = 7.2 Hz, 1H), 3.92 (s, 3H), 3.83 (s, 3H), 3.75 (s, 3H), 1.48 (d, J = 6.4 Hz, 3H), 1.31 (s, 9H). |

### Example 304

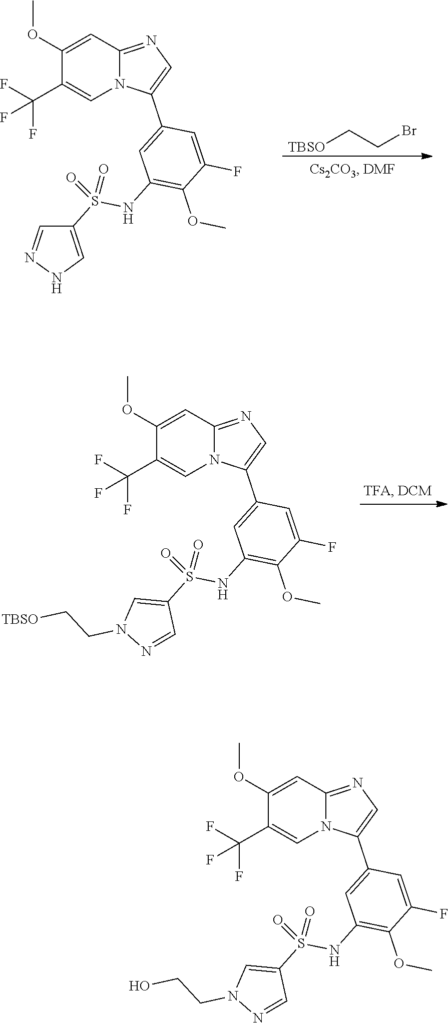 [SMILES: COc1cc2ncc(-c3cc(F)c(OC)c(NS(=O)(=O)c4cn[nH]c4)c3)n2cc1C(F)(F)F ; CC(C)(C)[Si](C)(C)OCCBr ; COc1cc2ncc(-c3cc(F)c(OC)c(NS(=O)(=O)c4cnn(CCO[Si](C)(C)C(C)(C)C)c4)c3)n2cc1C(F)(F)F ; COc1cc2ncc(-c3cc(F)c(OC)c(NS(=O)(=O)c4cnn(CCO)c4)c3)n2cc1C(F)(F)F]

1-(2-((tert-butyldimethylsilyl)oxy)ethyl)-N-(3-fluoro-2-methoxy-5-(7-methoxy-6-(trifluoromethyl)imidazo[1,2-a]pyridin-3-yl)phenyl)-1H-pyrazole-4-sulfonamide: A mixture of N-(3-fluoro-2-methoxy-5-(7-methoxy-6-(trifluoromethyl)imidazo[1,2-a]pyridin-3-yl)phenyl)-1H-pyrazole-4-sulfonamide (20 mg, 41.20 μmol, 1 eq), (2-bromoethoxy)(tert-butyl)dimethylsilane (11.83 mg, 49.44 μmol, 1.2 eq) and Cs₂CO₃ (40.27 mg, 123.61 μmol, 3 eq) in DMF (1 mL) was stirred at 50° C. for 2 h under N₂ atmosphere. The reaction mixture was diluted with brine (10 mL) and extracted with EtOAc (3×10). The combined organic layers were dried over Na₂SO₄, filtered and concentrated in vacuo to yield the title compound (15 mg, crude) as a yellow oil, which was carried on to the next step without additional purification [M+H]⁺=644.1.

N-(3-fluoro-2-methoxy-5-(7-methoxy-6-(trifluoromethyl)imidazo[1,2-a]pyridin-3-yl)phenyl)-1-(2-hydroxyethyl)-1H-pyrazole-4-sulfonamide: To a mixture of 1-(2-((tert-butyldimethylsilyl)oxy)ethyl)-N-(3-fluoro-2-methoxy-5-(7-methoxy-6-(trifluoromethyl)imidazo[1,2-a]pyridin-3-yl)phenyl)-1H-pyrazole-4-sulfonamide (15 mg, 23.30 μmol, 1 eq) in DCM (5 mL) was added TFA (1.50 mL, 20.19 mmol, 866.59 eq), and the mixture was stirred at 25° C. for 1 h under N₂ atmosphere. The reaction mixture was concentrated under reduced pressure to give a residue, which was purified by prep-HPLC (acidic conditions) to yield the title compound (6.44 mg, 12.06 μmol, 93.26% yield, 99.12% purity) as a yellow solid. [M+H]⁺=530.0. ¹H NMR (400 MHz, DMSO-d₆) δ ppm: 9.98 (s, 1H), 8.75 (s, 1H), 8.21 (s, 1H), 7.97 (s, 1H), 7.74 (s, 1H), 7.58-7.43 (m, 2H), 7.42 (s, 1H), 4.15 (t, J=5.2 Hz, 2H), 4.05 (s, 3H), 3.71-3.66 (s, 5H).

### Biological Data

### RIPK2 Binding Competition Assay

The ability of selected compounds of Formula (I) to inhibit the binding of an Alexa647-labelled ATP-competitive kinase inhibitor to a GST-RIPK2 fusion protein was quantified employing the TR-FRET-based RIPK2 binding competition assay as described in the following paragraphs.

Recombinant fusion protein of N-terminal Glutathione-S-Transferase (GST) and a fragment of human RIPK2 (amino acids 1-310 of accession number 043353), expressed in baculovirus infected Sf9 cells, purified via glutathione affinity chromatography, was used as GST-RIPK2 fusion protein. Tracer 199 from Life Technologies (catalogue no. PR9115B) was used as Alexa647-labelled ATP-competitive kinase inhibitor.

For the assay, 50 nL of a 100-fold concentrated solution of each test compound in DMSO was pipetted into either a black low volume 384-well microtiter plate or a black 1536-well microtiter plate (both Greiner Bio-One, Frickenhausen, Germany), 3 μL solution of Tracer 199 (16.7 nM=>final concentration in 5 μL assay volume is 10 nM) in aqueous assay buffer (25 mM Tris/HCl pH 7.5, 10 mM magnesium chloride (MgCl₂), 5 mM β-glycerophosphate, 2.5 mM dithiothreitol (DTT), 0.5 mM ethylene glycol-bis(2-aminoethylether)-N,N,N′,N′-tetraacetic acid (EGTA), 0.5 mM sodium ortho-vanadate, 0.01% (w/v) bovine serum albumin (BSA), 0.005% (w/v) Pluronic F-127 (Sigma)) were added. Then the binding competition was started by the addition of 2 μL of a solution of the GST-RIPK2 fusion protein (2.5 nM=>final conc. in the 5 μL assay volume is 1 nM) and of Anti-GST-Tb (1.25 nM=>final conc. in the 5 μL assay volume is 0.5 nM), a Lumi4®—Tb cryptate-conjugated anti-GST antibody from PerkinElmer (catalogue no 61GSTTAH), in assay buffer.

The resulting mixture was incubated 45 min at 22° C. to allow the formation of a complex between the Tracer 199, the fusion protein and Anti-GST-Tb. Subsequently, the amount of this complex was evaluated by measurement of the resonance energy transfer from the Tb-cryptate to the Tracer 199. Therefore, the fluorescence emissions at 620 nm and 665 nm after excitation at 337 nm were measured in a TR-FRET reader, e.g. a Pherastar FS (BMG Labtechnologies, Offenburg, Germany) or a Viewlux (PerkinElmer). The ratio of the emissions at 665 nm and at 620 nm was taken as the measure of the amount of the complex. The data were normalized (assay reaction without inhibitor=0% activity, all other assay components but no GST-RIPK2 fusion protein=−100% activity). Typically the test compounds were tested on the same microtiter plate in 11 different concentrations in the range of 20 μM to 0.07 nM (20 μM, 5.7 μM, 1.6 μM, 0.47 μM, 0.13 μM, 38 nM, 11 nM, 3.1 nM, 0.9 nM, 0.25 nM and 0.07 nM, is the dilution series prepared separately before the assay on the level of the 100-fold concentrated solutions in DMSO by serial dilutions; exact concentrations may vary depending on pipettors used) in duplicate values for each concentration and IC₅₀ values were calculated using Genedata Screener™ software.

Table A1 below lists IC₅₀ values of selected compounds of Formula (I) measured in the RIPK2 binding competition assay. For the RIPK2 competition assay IC₅₀ values, “A” denotes an IC₅₀ of <1 nM; “B” denotes an IC₅₀ of 1 nM≤B≤2.5; “C” denotes an IC₅₀ of 2.5 nM<C≤20; and “D” denotes an IC₅₀ of 20 nM<D.

TABLE A1
IC₅₀ Values

| Ex. No. | RIPK2 IC₅₀ (nM) |
|---|---|
| 1 | C |
| 2 | C |
| 3 | D |
| 4 | D |
| 5 | D |
| 6 | C |
| 7 | D |
| 8 | D |
| 9 | D |
| 10 | D |
| 11 | C |
| 12 | C |
| 13 | B |
| 14 | B |
| 15 | B |
| 16 | C |
| 17 | B |
| 18 | C |
| 19 | C |
| 20 | C |
| 21 | B |
| 22 | C |
| 23 | C |
| 24 | C |
| 25 | C |
| 26 | B |
| 27 | D |
| 28 | B |
| 29 | C |
| 30 | C |
| 31 | C |
| 32 | B |
| 33 | C |
| 34 | C |
| 35 | C |
| 36 | B |
| 37 | D |
| 38 | B |
| 39 | D |
| 40 | D |
| 41 | C |
| 42 | D |
| 43 | C |
| 44 | D |
| 45 | B |
| 46 | B |
| 47 | B |
| 48 | B |
| 49 | C |
| 50 | D |
| 51 | D |
| 52 | A |
| 53 | A |
| 54 | B |
| 55 | C |
| 56 | C |
| 57 | C |
| 58 | D |
| 59 | A |
| 60 | C |
| 61 | D |
| 62 | C |
| 63 | C |
| 64 | D |
| 65 | D |
| 66 | D |
| 67 | A |
| 68 | D |
| 69 | C |
| 70 | D |
| 71 | C |
| 72 | D |
| 73 | C |
| 74 | D |
| 75 | D |
| 76 | D |
| 77 | D |
| 78 | C |
| 79 | D |
| 80 | D |
| 81 | B |
| 82 | D |
| 83 | D |
| 84 | D |
| 85 | D |
| 86 | D |
| 87 | D |
| 88 | D |
| 89 | C |
| 90 | C |
| 91 | D |
| 92 | D |
| 93 | C |
| 94 | C |
| 95 | C |
| 96 | C |
| 97 | C |
| 98 | C |
| 99 | C |
| 100 | C |
| 101 | C |
| 102 | D |
| 103 | D |
| 104 | D |
| 105 | D |
| 106 | D |
| 107 | D |
| 108 | D |
| 109 | D |
| 110 | D |
| 111 | D |
| 112 | D |
| 113 | D |
| 114 | D |
| 115 | D |
| 116 | D |
| 117 | C |
| 118 | B |
| 119 | A |
| 120 | C |
| 121 | D |
| 122 | D |
| 123 | D |
| 124 | C |
| 125 | C |
| 126 | A |
| 127 | B |
| 128 | B |
| 129 | A |
| 130 | A |
| 131 | C |
| 132 | A |
| 133 | A |
| 134 | B |
| 135 | A |
| 141 | A |
| 142 | B |
| 143 | B |
| 144 | B |
| 145 | A |
| 146 | C |
| 147 | C |
| 148 | C |
| 149 | C |
| 150 | C |
| 151 | B |
| 152 | C |
| 153 | C |
| 154 | C |
| 155 | D |
| 156 | D |
| 157 | D |
| 158 | D |
| 159 | A |
| 160 | A |
| 161 | D |
| 162 | B |
| 163 | D |
| 164 | D |
| 165 | D |
| 166 | C |
| 167 | D |
| 168 | C |
| 169 | B |
| 170 | B |
| 171 | C |
| 172 | C |
| 173 | C |
| 174 | B |
| 175 | B |
| 176 | B |
| 177 | B |
| 178 | A |
| 179 | C |
| 180 | B |
| 181 | A |
| 182 | A |
| 183 | A |
| 184 | B |
| 185 | C |
| 186 | C |
| 187 | C |
| 188 | D |
| 189 | D |
| 190 | D |
| 191 | C |
| 192 | C |
| 193 | C |
| 194 | A |
| 195 | A |
| 196 | B |
| 197 | A |
| 198 | B |
| 199 | A |
| 200 | A |
| 201 | B |
| 202 | B |
| 203 | B |
| 204 | B |
| 205 | D |
| 206 | B |
| 207 | C |
| 208 | C |
| 209 | D |
| 210 | D |
| 211 | C |
| 212 | B |
| 213 | D |
| 214 | A |
| 215 | A |
| 216 | A |
| 217 | A |
| 218 | C |
| 219 | B |
| 220 | A |
| 221 | D |
| 222 | C |
| 223 | A |
| 224 | A |
| 225 | A |
| 226 | A |
| 227 | B |
| 228 | A |
| 229 | D |
| 230 | D |
| 231 | D |
| 232 | C |
| 233 | C |
| 234 | A |
| 235 | D |
| 236 | A |
| 237 | D |
| 238 | B |
| 239 | C |
| 240 | B |
| 241 | C |
| 242 | A |
| 243 | B |
| 244 | B |
| 245 | B |
| 246 | B |
| 247 | B |
| 248 | C |
| 249 | D |
| 250 | C |
| 251 | A |
| 252 | A |
| 253 | B |
| 254 | B |
| 255 | B |
| 256 | B |
| 257 | C |
| 258 | C |
| 259 | C |
| 276 | B |
| 277 | C |
| 278 | D |
| 279 | A |
| 280 | B |
| 281 | A |
| 295 | C |
| 296 | D |
| 297 | B |
| 298 | C |
| 299 | D |
| 304 | C |

### RIPK2-XIAP Binding Competition PPI Assay

The ability of selected compounds of Formula (I) to inhibit the binding of an His-XIAP(BIR2)-avi fusion protein to a GST-RIPK2 fusion protein was quantified employing the TR-FRET-based competition assay as described in the following paragraphs.

Recombinant fusion protein of N-terminal Glutathione-S-Transferase (GST) and a fragment of human RIPK2 (amino acids 1-310 of accession number 043353), expressed in baculovirus infected Sf9 cells, purified via glutathione affinity chromatography, was used as GST-RIPK2 fusion protein. Recombinant fusion protein of N-terminal 6×Histidine (His), a fragment of human XIAP (amino acids 152-231 of accession number P98170), and C-terminal avi tag, expressed in baculovirus infected Sf9 cells, purified via Nickel NTA affinity chromatography, was used as the His-XIAP(BIR2)-avi fusion protein.

For the assay, a working solution of 1 nM GST-RIPK2, 25 nM His-XIAP(BIR2)-avi, 6 nM Anti-His-XL665 nM (Revvity Catalogue No. 61HISXLB), and 0.5 nM Anti-GST-Tb (Revvity Catalogue No. 61GSTTLB) in 25 mM Tris-HCl 7.5, 0.5 mM ethylene glycol-bis(2-aminoethylether)-N,N,N′,N′-tetraacetic acid (EGTA), 10 mM magnesium chloride (MgCl2), 2.5 mM dithiothreitol (DTT), 0.01% (w/v) bovine serum albumin (BSA), 0.005% (w/v) Pluronic F-127 (Sigma), 0.5 mM sodium ortho-vanadate, 5 mM β-Glycerophosphate disodium salt hydrate (BGP) was prepared. 50 nL of a 100-fold concentrated solution of each test compound in DMSO was dispensed via acoustic dispensing into a white low volume 384-well microtiter plate (Perkin Elmer Catalogue No. 6008280). Subsequently, 5 μL of the assay working solution were added.

The resulting mixture was incubated 120 min at 23° C. Subsequently, the amount of complex formed by the GST-RIPK2, XIAP(BIR2)-avi, Anti-His-XL665, and Anti-GST-Tb was evaluated by measurement of the resonance energy transfer from the Tb-cryptate to the XL665. Therefore, the fluorescence emissions at 520 nm and 665 nm after excitation at 337 nm were measured in a TR-FRET reader, e.g. a Pherastar FS (BMG Labtechnologies, Offenburg, Germany) or an Envision (PerkinElmer). The ratio of the emissions at 665 nm and at 520 nm was taken as the measure of the amount of the complex. The data were normalized (assay reaction without inhibitor=0% activity, 50 nM of GSK583=−100% activity). Typically the test compounds were tested on the same microtiter plate in 11 different concentrations in the range of 10 μM to 0.04 nM (10 μM, 2.86 μM, 0.82 μM, 0.23 μM, 67 nM, 19 nM, 5.4 nM, 1.6 nM, 0.4 nM, 0.1 nM and 0.04 nM, is the dilution series prepared separately before the assay on the level of the 100-fold concentrated solutions in DMSO by serial dilutions; exact concentrations may vary depending on pipettors used) in duplicate values for each concentration and IC₅₀ values were calculated using Collaborative Drug Discovery software.

### THP-1 Dual Cell SEAP and Viability Assays

THP1-Dual™ cells (InvivoGen Cat #thpd-nfis) allow simultaneous assessment of the NF-κB pathway, by monitoring the activity of secreted embryonic alkaline phosphatase (SEAP) as well as the IRF pathway, by assessing the activity of a secreted luciferase (Lucia). Cells were cultured according to manufacturers' recommendations. For the assay, cell concentration was adjusted to 7.14×10⁵ cells/mL in assay media (RPMI 1640, 2 mM L-Glutamine, 25 mM HEPES, 10% fetal bovine serum (heat-inactivated for 30 min at 56° C.), Pen-Strep (100 U/mL)) and 35 L of cell suspension (˜25,000 cells) per well were added to a flat bottom 384-well plate (white opaque). Plate was centrifuged at 300 g for 2 min, compounds were added in serial dilution series according to the plate layout (volumes ˜500 nL-0.0152 nL) and plate was incubated at 37° C. in 5% CO₂ for 30 min. Using assay media, a L18-MDP stock solution, that results in a final concentration of 10 ng/mL L18-MDP in each well when adding 15 μL to each well, was prepared. 15 μL of L18-MDP stock solution was added to each well, while 15 μL of assay media was added to the negative control wells. Plates were subsequently incubated at 37° C. in 5% CO₂ overnight (20-24 h).

For the SEAP assay: without disturbing the cells at the bottom of the well, 15 μL supernatant was transferred to a clear 384-well flat bottom plate and 35 μL QUANTI-Blue Solution per well was added. Following a 2 h incubation at 37° C., the optical density (OD) was measured at 620-655 nm using a microplate reader (PheraStar FS microplate reader with protocol for OD 620-655 nm).

For the viability assay: the cells remaining in the plate were used to assess cell viability. For this, CellTiter-Glo buffer and lyophilized CellTiter-Glo substrate was allowed to equilibrate to room temperature and lyophilized CellTiter-Glo substrate was reconstituted according to the manufacturers' recommendations. The equal volume (here 35 μL) of CTG was added to each well, contents were mixed on an orbital shaker for 2 min to induce lysis followed by a 10 min incubation at room temperature. Luminescence signal was subsequently measured in a microplate reader (PheraStar).

### Analysis

Values of media-only wells were subtracted and % inhibition for each compound concentration relative to the DMSO/L18-MDP-treated controls was calculated. Inhibition values+/−SD were fitted by non-linear regression using Prism software (GraphPad PRISM Software) to calculate IC₅₀ values.

Table A below lists potency values of selected compounds of Formula (I) measured in the RIPK2/XIAP binding competition PPI assay and the THP-1 Dual cell SEAP and viability assays.

For the RIPK2/XIAP binding competition PPI assay IC₅₀ values, “A” denotes an IC₅₀ of <1 nM; “B” denotes an IC₅₀ of 1 nM≤B≤2.5; “C” denotes an IC₅₀ of 2.5 nM<C≤20; and “D” denotes an IC₅₀ of 20 nM<D.

For the RIPK2/XIAP binding competition PPI assay Aₘₐₓ values, “A” denotes an Aₘₐₓ of ≥80%; “B” denotes an Aₘₐₓ of 80%>B≥55; “C” denotes an Aₘₐₓ of 55%>C>30; and “D” denotes an Aₘₐₓ of ≤30%.

For the THP-1 assay IC₅₀ values, “A” denotes an IC₅₀ of <50 nM; “B” denotes an IC₅₀ of 50 nM≤B≤250; “C” denotes an IC₅₀ of 250 nM<C≤1,000; and “D” denotes an IC₅₀ of 1,000 nM<D.

TABLE A
Potency Values

| Ex. No. | PPI IC₅₀ bin | PPI Aₘₐₓ bin | NF-κB THP1 IC₅₀ bin |
|---|---|---|---|
| 1 | D | D | B |
| 2 | D | D | B |
| 3 | D | D | C |
| 4 | D | D | C |
| 5 | D | D | C |
| 6 | D | D | B |
| 7 | D | C | D |
| 8 | D | D | D |
| 9 | D | D | D |
| 10 | D | A | |
| 11 | D | D | A |
| 12 | C | A | B |
| 13 | B | A | B |
| 14 | B | A | B |
| 15 | B | A | B |
| 16 | C | A | C |
| 17 | B | A | B |
| 18 | C | A | C |
| 19 | C | A | C |
| 20 | D | A | D |
| 21 | C | A | D |
| 22 | C | A | C |
| 23 | C | A | C |
| 24 | C | A | B |
| 25 | C | A | C |
| 26 | C | A | B |
| 27 | D | A | D |
| 28 | C | A | B |
| 29 | C | A | C |
| 30 | C | A | B |
| 31 | C | A | D |
| 32 | C | A | B |
| 33 | C | A | D |
| 34 | C | A | C |
| 35 | C | A | C |
| 36 | C | A | B |
| 37 | D | A | D |
| 38 | C | A | B |
| 39 | D | A | D |
| 40 | D | B | D |
| 41 | C | A | D |
| 42 | D | B | D |
| 43 | C | A | D |
| 44 | D | A | D |
| 45 | B | A | B |
| 46 | B | A | B |
| 47 | B | A | B |
| 48 | C | A | B |
| 49 | C | A | C |
| 50 | D | B | D |
| 51 | D | D | D |
| 52 | A | A | A |
| 53 | B | A | B |
| 54 | C | A | B |
| 55 | C | A | C |
| 56 | C | A | B |
| 57 | C | A | C |
| 58 | D | A | D |
| 59 | A | A | A |
| 60 | C | A | C |
| 61 | D | A | D |
| 62 | C | A | C |
| 63 | C | A | D |
| 64 | D | A | D |
| 65 | D | A | D |
| 66 | D | A | D |
| 67 | A | A | A |
| 68 | D | A | D |
| 69 | C | A | C |
| 70 | D | A | D |
| 71 | C | A | D |
| 72 | D | A | D |
| 73 | C | A | C |
| 74 | D | A | D |
| 75 | D | A | D |
| 76 | D | A | D |
| 77 | D | A | D |
| 78 | C | A | D |
| 79 | D | A | D |
| 80 | D | D | D |
| 81 | D | D | C |
| 82 | D | D | D |
| 83 | D | D | D |
| 84 | D | B | D |
| 85 | D | C | D |
| 86 | D | D | D |
| 87 | D | C | D |
| 88 | D | D | D |
| 89 | D | D | C |
| 90 | D | D | C |
| 91 | D | C | D |
| 92 | D | C | D |
| 93 | D | D | C |
| 94 | D | D | C |
| 95 | D | D | D |
| 96 | D | D | C |
| 97 | D | D | D |
| 98 | D | D | D |
| 99 | D | D | D |
| 100 | D | D | D |
| 101 | D | D | D |
| 102 | D | D | D |
| 103 | D | D | D |
| 104 | D | C | D |
| 105 | D | C | D |
| 106 | D | D | D |
| 107 | D | B | D |
| 108 | D | D | D |
| 109 | D | D | D |
| 110 | D | D | D |
| 111 | D | D | D |
| 112 | D | D | D |
| 113 | D | D | D |
| 114 | D | D | D |
| 115 | D | B | D |
| 116 | D | B | D |
| 117 | C | C | C |
| 118 | A | B | B |
| 119 | A | B | A |
| 120 | C | B | D |
| 121 | D | B | D |
| 122 | D | D | D |
| 123 | D | C | D |
| 124 | D | A | D |
| 125 | C | A | C |
| 126 | A | A | A |
| 127 | B | A | B |
| 128 | B | B | B |
| 129 | A | A | B |
| 130 | A | B | A |
| 131 | C | A | C |
| 132 | A | A | A |
| 133 | A | B | A |
| 134 | A | A | A |
| 135 | A | A | A |
| 136 | C | A | C |
| 137 | C | A | C |
| 138 | A | A | A |
| 139 | D | B | D |
| 140 | A | A | A |
| 141 | A | A | A |
| 142 | B | A | C |
| 143 | B | B | C |
| 144 | A | A | A |
| 145 | A | A | A |
| 146 | C | A | D |
| 147 | C | A | C |
| 148 | C | A | D |
| 149 | C | A | D |
| 150 | C | A | D |
| 151 | B | A | C |
| 152 | C | A | C |
| 153 | D | A | D |
| 154 | C | A | D |
| 155 | D | A | D |
| 156 | D | A | D |
| 157 | D | B | D |
| 158 | D | A | D |
| 159 | A | A | A |
| 160 | A | A | A |
| 161 | D | A | D |
| 162 | B | B | B |
| 163 | D | A | D |
| 164 | D | A | D |
| 165 | D | A | D |
| 166 | C | A | D |
| 167 | D | A | D |
| 168 | C | A | C |
| 169 | B | A | B |
| 170 | B | A | B |
| 171 | C | A | B |
| 172 | C | A | C |
| 173 | C | A | D |
| 174 | B | A | B |
| 175 | B | A | B |
| 176 | B | A | B |
| 177 | C | A | B |
| 178 | A | A | A |
| 179 | C | B | D |
| 180 | A | A | B |
| 181 | A | A | A |
| 182 | A | B | A |
| 183 | A | B | A |
| 184 | B | C | C |
| 185 | B | C | B |
| 186 | C | A | C |
| 187 | C | A | C |
| 188 | D | A | D |
| 189 | D | A | D |
| 190 | D | B | D |
| 191 | C | A | D |
| 192 | C | A | D |
| 193 | D | A | D |
| 194 | B | A | B |
| 195 | A | A | A |
| 196 | B | A | B |
| 197 | A | A | B |
| 198 | A | A | B |
| 199 | A | A | B |
| 200 | A | A | A |
| 201 | B | A | B |
| 202 | B | A | B |
| 203 | B | A | B |
| 204 | B | A | B |
| 205 | D | B | D |
| 206 | A | B | B |
| 207 | D | D | D |
| 208 | D | D | D |
| 209 | D | C | D |
| 210 | D | D | D |
| 211 | C | A | D |
| 212 | B | A | B |
| 213 | D | B | D |
| 214 | A | A | B |
| 215 | A | A | A |
| 216 | A | A | A |
| 217 | A | B | B |
| 218 | C | B | D |
| 219 | B | A | B |
| 220 | A | A | A |
| 221 | D | A | D |
| 222 | D | D | D |
| 223 | A | B | B |
| 224 | A | A | A |
| 225 | A | A | A |
| 226 | A | A | A |
| 227 | A | A | A |
| 228 | A | A | A |
| 229 | D | D | D |
| 230 | D | D | D |
| 231 | D | C | D |
| 232 | A | C | D |
| 233 | D | D | D |
| 234 | A | A | A |
| 235 | D | D | D |
| 236 | A | A | A |
| 237 | D | D | D |
| 238 | B | A | B |
| 239 | C | A | C |
| 240 | B | A | B |
| 241 | C | A | C |
| 242 | A | A | B |
| 243 | B | A | B |
| 244 | A | A | A |
| 245 | A | A | A |
| 246 | B | A | B |
| 247 | B | A | B |
| 248 | C | B | D |
| 249 | D | A | D |
| 250 | D | A | D |
| 251 | A | A | A |
| 252 | A | A | A |
| 253 | A | A | A |
| 254 | B | A | B |
| 255 | A | B | B |
| 256 | A | A | A |
| 257 | C | A | C |
| 258 | C | A | C |
| 259 | B | A | B |
| 260 | B | A | B |
| 261 | B | A | B |
| 262 | A | A | A |
| 263 | A | A | A |
| 264 | A | A | A |
| 265 | B | A | B |
| 266 | B | A | B |
| 267 | C | A | D |
| 268 | A | A | A |
| 269 | A | B | A |
| 270 | A | B | A |
| 271 | C | A | D |
| 272 | B | A | B |
| 273 | B | A | C |
| 274 | C | A | C |
| 275 | B | A | B |
| 276 | A | A | B |
| 277 | D | D | C |
| 278 | C | C | D |
| 279 | A | A | A |
| 280 | B | A | B |
| 281 | A | A | A |
| 282 | A | A | A |
| 283 | A | A | A |
| 284 | A | A | B |
| 285 | A | A | A |
| 286 | A | A | B |
| 287 | A | B | B |
| 288 | D | D | D |
| 289 | A | C | B |
| 290 | D | D | D |
| 291 | A | B | A |
| 292 | A | C | B |
| 293 | A | C | A |
| 294 | A | A | B |
| 295 | C | A | D |
| 296 | D | A | D |
| 297 | B | A | B |
| 298 | C | B | C |
| 299 | D | A | D |
| 300 | C | A | C |
| 301 | A | B | B |
| 302 | A | A | A |
| 303 | C | A | C |
| 304 | C | A | D |

What is claimed is:

1. A compound of Formula (I): 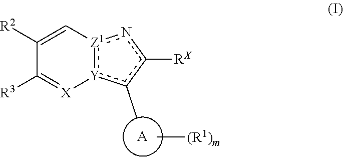
or a pharmaceutically acceptable salt thereof, wherein:
one or two of X, Y, and Z¹ is independently N and the other of X, Y, and Z¹ is C;
each [symbol] is a single bond or a double bond;
R^{X} is hydrogen, —NH₂, or halogen;
Ring A is phenyl or 5-11 membered heteroaryl;
m is 0, 1, 2, 3, or 4;
each R¹ is independently:
(i) C1-C6 alkoxy optionally substituted with hydroxyl or phenyl,
(ii) C1-C6 deuteroalkoxy,
(iii) C1-C6 alkoxyalkyl,
(iv) 5-6 membered heteroaryl,
(v) —N(R^{1A})—S(O₂)R^{1B},
(vi) —(C═O)NR^{1A}R^{1B},
(vii) halogen,
(viii) cyano,
(ix) hydroxyl,
(x) —NR^{A}R^{B},
(xi) C1-C6 alkyl optionally substituted with 1-2 substituents independently selected from hydroxyl and 4-8 membered heterocyclyl optionally substituted with hydroxyl or C1-C6 alkyl,
(xii) C1-C6 haloalkyl,
(xiii) C1-C6 haloalkoxy,
(xiv) C3-C6 cycloalkyl,
(xv) 4-8 membered heterocyclyl optionally substituted with 1-2 substituents independently selected from hydroxyl, C1-C6 alkyl, C1-C6 haloalkyl, and —(C═O)OC1-C6 alkyl,
(xvi) —S(O₂)C1-C6 alkyl,
(xvii) 4-10 membered heterocyclyloxy optionally substituted with acyl, or
(xviii) phenyl;
each R^{1A} is independently hydrogen or C1-C6 alkyl;
each R^{1B} is independently
(i) C1-C6 alkyl optionally substituted with C3-C6 cycloalkyl, 5-6 membered heteroaryl optionally substituted with C1-C6 alkyl, or 4-10 membered heterocyclyl optionally substituted with —(C═O)OC1-C6 alkyl,
(ii) 5-10 membered heteroaryl optionally substituted with 1-3 substituents independently selected from C1-C6 alkyl, hydroxyl, and C1-C6 hydroxyalkyl,
(iii) C3-C6 cycloalkyl optionally substituted with 1-2 substituents independently selected from C1-C6 alkyl and C1-C6 hydroxyalkyl,
(iv) ethylenyl,
(v) C1-C6 haloalkyl,
(vi) 4-10 membered heterocyclyl optionally substituted with —(C═O)OC1-C6 alkyl,
(vii) —NR^{1A}R^{1A}, or
(viii) phenyl;
R^{A} is hydrogen or C1-C6 alkyl;
R^{B} is
(i) hydrogen,
(ii) —S(O₂)C1-C6 alkyl,
(iii) C3-C6 cycloalkyl optionally substituted with hydroxyl or C1-C6 alkoxy,
(iv) —(C═O)C1-C6 alkyl,
(v) —(C═O)OC1-C6 alkyl,
(vi) 4-8 membered heterocyclyl optionally substituted with hydroxyl,
(vii) C1-C6 alkyl optionally substituted with 1-4 substituents independently selected from: halogen, hydroxyl, —NR^{C}R^{D}, C1-C6 alkoxy, C1-C6 haloalkoxy, C3-C6 cycloalkyl, phenyl optionally substituted with C1-C6 alkoxy, 5-6 membered heteroaryl optionally substituted with C1-C6 alkyl, and 4-8 membered heterocyclyl optionally substituted with —C(═O)C1-C6 alkyl or C1-C6 alkyl, or
(viii) —(C═O)-5- or 6-membered heteroaryl optionally substituted with C1-C6 alkyl;
R² is
(i) hydrogen,
(ii) halogen,
(iii) C1-C6 alkoxy optionally substituted with 1-3 substituents independently selected from
(a) hydroxyl,
(b) halogen,
(c) phosphate,
(d) —NR^{2A}R^{2B},
(e) 4-10 membered heterocyclyl optionally substituted with 1-3 substituents independently selected from hydroxyl, C1-C6 alkyl, C1-C6 haloalkyl, C1-C6 hydroxyalkyl, C1-C6 alkoxy, and C1-C6 alkoxyalkyl,
(f) 5-6 membered heteroaryl optionally substituted with C1-C6 alkyl,
(g) C3-C6 cycloalkyl optionally substituted with hydroxyl or hydroxyalkyl,
(h) CO₂H,
(i) C(═O)R^{2A}
(j) C1-C6 alkoxy; or
(k) oxo;
(iv) C1-C6 haloalkoxy,
(v) 4-10 membered heterocyclyloxy,
(vi) —(C═O)NR^{2A}R^{2B},
(vii) C1-C6 alkyl optionally substituted with 1-3 substituents independently selected from hydroxyl, halogen, and —NR^{2A}R^{2B},
(viii) 5-6 membered heteroaryl optionally substituted with C1-C6 alkyl or C1-C6 alkoxy, or
(ix) —NR^{2A}R^{2B};
each R^{2A} and R^{2B} are independently hydrogen, C1-C6 alkyl optionally substituted with hydroxyl, or C1-C6 hydroxyalkyl;
R³ is
(i) C1-C6 thioalkyl, 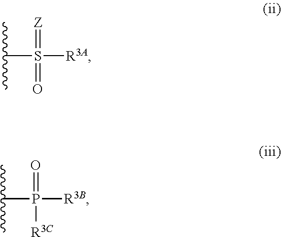
(iv) 4-8 membered heterocyclyl optionally substituted with 1-3 substituents independently selected from halogen, hydroxyl, C1-C6 alkyl, and C1-C6 alkoxy,
(v) C1-C6 alkyl optionally substituted with NR^{E}R^{F} or hydroxyl,
(vi) —CO₂H,
(vii) —C(═O)NR^{E}R^{F},
(viii) C1-C6 alkoxy optionally substituted with 4-10 membered heterocyclyl optionally substituted with C1-C6 alkoxy,
(ix) hydrogen,
(x) C1-C6 haloalkyl,
(xi) 5-6 membered heteroaryl optionally substituted with C1-C6 alkyl,
(xii) C1-C6 alkoxyalkyl, or
(xiii) C1-C6 hydroxyalkyl;
Z is O or NR⁴;
R^{3A} is
(i) C1-C6 haloalkyl,
(ii) C3-C6 cycloalkyl optionally substituted with C1-C6 alkyl,
(iii) C1-C6 alkyl optionally substituted with
(a) C3-C6 cycloalkyl,
(b) 5-6 membered heteroaryl optionally substituted with C1-C6 alkyl,
(c) hydroxyl,
(d) C1-C6 alkoxy, or
(e) 4-6 membered heterocyclyl optionally substituted with 4-6 membered heterocyclyl or C1-C6 alkyl optionally substituted with C1-C6 alkoxy,
(iv) C1-C6 alkoxyalkyl,
(v) C1-C6 hydroxyalkyl,
(vi) 5-6 membered heteroaryl optionally substituted with C1-C6 alkyl,
(vii) 4-10 membered heterocyclyl optionally substituted with 1-3 substituents independently selected from hydroxyl, C1-C6 alkyl, —C(O)OC1-C6 alkyl, and C1-C6 alkoxy, or
(viii) —N(C1-C6 alkyl)₂;
R^{3B} and R^{3C} are each independently C3-C6 cycloalkyl or C1-C6 alkyl optionally substituted with C3-C6 cycloalkyl, or
R^{3B} and R^{3C} with the atom to which they are attached together form a 4-8 membered heterocyclyl optionally substituted with C1-C6 alkyl;
R⁴ is hydrogen or C1-C6 alkyl; and
each R^{C} and R^{D} are each independently hydrogen, —(C═O)C1-C6 alkyl, or C1-C6 alkyl optionally substituted with oxo;
each R^{E} and R^{F} are each independently hydrogen or C1-C6 alkyl, or R^{E} and R^{F} with the atom to which they are attached together form a 4-8 membered heterocyclyl optionally substituted with hydroxyl.

2. The compound of claim 1, wherein Formula (I) is (I-a): 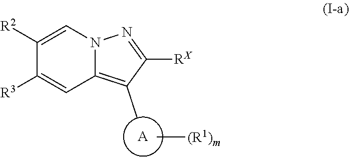
or a pharmaceutically acceptable salt thereof.

3. The compound of claim 1, wherein Formula (I) is (I-b): 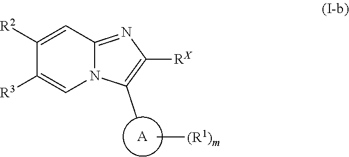
or a pharmaceutically acceptable salt thereof.

4. The compound of claim 1, wherein Formula (I) is (I-c): 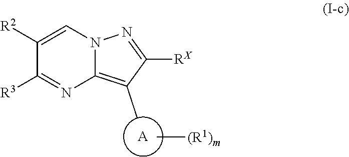
or a pharmaceutically acceptable salt thereof.

5. The compound of claim 1, wherein Formula (I) is (I-d): 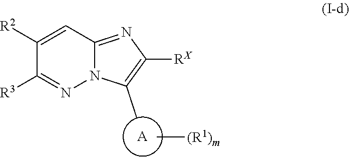
or a pharmaceutically acceptable salt thereof.

6. The compound of claim 1, wherein Formula (I) is (I-e): 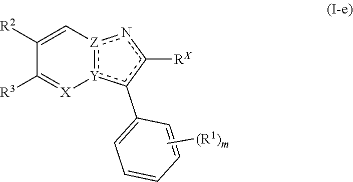
or a pharmaceutically acceptable salt thereof.

7. The compound of claim 1, wherein Formula (I) is (I-f): 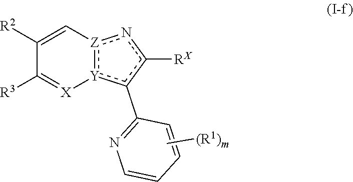
or a pharmaceutically acceptable salt thereof.

8. The compound of claim 1, wherein Formula (I) is (I-g): 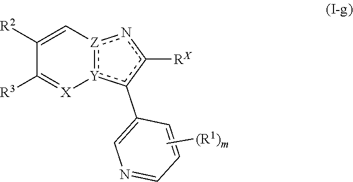
or a pharmaceutically acceptable salt thereof.

9. The compound of claim 1, wherein Formula (I) is (I-i): 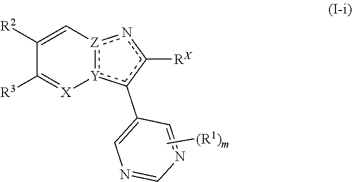
or a pharmaceutically acceptable salt thereof.

10. The compound of claim 1, wherein Formula (I) is (I-n): 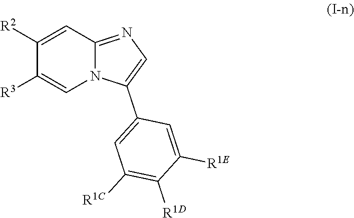
or a pharmaceutically acceptable salt thereof, wherein R^{1C}, R^{1D}, and R^{1E} are each an independently selected R¹.

11. The compound of claim 1, wherein Formula (I) is (1-0): 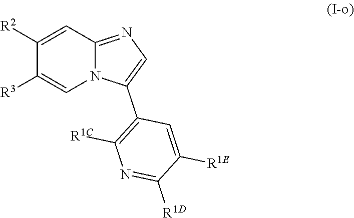
or a pharmaceutically acceptable salt thereof, wherein R^{1C}, R^{1D}, and R^{1E} are each an independently selected R¹.

12. The compound of claim 1, wherein Formula (I) is (I-p): 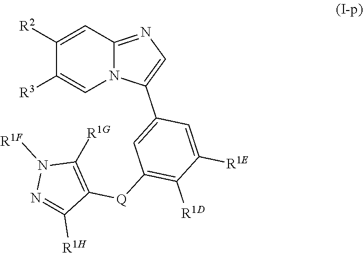
or a pharmaceutically acceptable salt thereof, wherein:
R^{1D} and R^{1E} are each an independently selected R¹;
Q is —NH(SO₂)— or —NH(C1-C6 alkyl)-; and
R^{1F}, R^{1G}, and R^{1H} are each independently hydrogen, C1-C6 alkyl, or C1-C6 hydroxyalkyl, wherein at least one of R^{1F}, R^{1G}, and R^{1H} is hydrogen.

13. The compound of any one of claims 1-9, wherein X is N or C.

14. The compound of any one of claims 1-9, wherein Y is N or C.

15. The compound of any one of claims 1-5, wherein Z¹ is N or C.

16. The compound of any one of claims 1-4, wherein Ring A is 5-11 membered heteroaryl.

17. The compound of any one of claims 1-4, wherein Ring A is phenyl.

18. A compound selected from the group consisting of the compounds in Examples 1-304, or a pharmaceutically acceptable salt thereof.

19. A pharmaceutical composition comprising a compound of any one of claims 1-18, or a pharmaceutically acceptable salt thereof, and one or more pharmaceutically acceptable excipients.

20. A method of treating a RIPK2-associated disease or disorder in a subject in need thereof, comprising administering to the subject an effective amount of a compound of any one of claims 1-18, or a pharmaceutically acceptable salt thereof, or the pharmaceutical composition of claim 19.
